SEQUENCE LISTING (378 sequences)

SEQ ID NO: 1 - protein, 537 aa, Arabidopsis thaliana
  source 1..537: note = Arabidopsis thaliana PPO1 (with transit peptide)
MELSLLRPTTQSLLPSFSKPNLRLNVYKPLRLRCSVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 2 - protein, 535 aa, Setaria italica
  source 1..535: note = Setaria italica PPO1 (with transit peptide)
MVAAAMATAPSAGVPPLRGTRGPARFRIRGVSVRCAAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 3 - DNA, 6857 bp, synthetic construct
  source 1..6857: mol_type = other DNA; note = pET24A AtPPO1
gcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatatacatatggaactttctttgttgcgcccgactacacaatccctgctgccctcttttagcaagccgaatctgcgtctgaatgtgtataagccactgcgcctgcgctgttctgtggcaggcgggccgacggtggggtcgtcgaagatcgagggtggcggcggcactacgattaccaccgactgcgttatcgtgggcgggggcatcagcggcttgtgcatcgcacaagccctggccacgaaacatcccgatgcagcacccaatctgatcgtaacggaagcgaaggatcgcgtgggtgggaacattattacacgcgaagagaatggttttctgtgggaagaagggcctaattcctttcagccaagcgatccgatgttaaccatggtagtggactcaggcctgaaagatgacctcgtgctcggcgacccgaccgcaccgcgtttcgtcctgtggaatggcaaacttcgcccggtgccgtctaaactgaccgacctgccgttctttgacctcatgtccatcgggggtaaaatccgtgctggctttggcgctctcgggattcgtccgtctccgccgggtcgcgaagaaagtgtagaagagttcgtccgccgtaatctgggcgatgaagtctttgagcgtctgatcgagccattctgtagtggtgtttatgctggtgatccgagtaaactgtccatgaaggccgcgtttggcaaggtgtggaaactggagcagaatggaggtagcatcatcgggggtacattcaaagcaattcaagaacggaaaaatgctcccaaagcagaacgtgatccgcggctcccgaaaccgcaaggtcagaccgttggttccttccgcaaaggacttcgcatgctgccggaagccatcagcgcgcgtttgggaagcaaagtaaaattgtcttggaaactgtcgggtattacgaaactggaatcggggggttataatttgacatatgaaaccccagatggtttggtgtcagtgcagagcaagagcgttgtcatgaccgtgccctcacatgtcgcctccgggttgttgcgtccgctgagcgaaagcgcagcgaacgccctctctaaactttactatccgcctgttgccgccgtgagcattagttatcctaaggaagctattcgcaccgaatgtctgattgatggtgagctcaagggttttggtcagctgcacccgcggactcagggcgtcgagactttaggtaccatctactcctcttctctgtttccgaaccgcgcaccgccgggccgcattctgctgctgaattatattggtggtagtacgaataccggaattttatcgaaaagtgaaggtgaattggtcgaagccgtcgatcgtgaccttcgcaaaatgctgattaaaccgaactcgaccgacccgctgaagttaggcgtgcgtgtgtggccccaagcgatcccacagtttctggttggtcacttcgatattctggataccgccaaatccagcctgacaagtagcggctatgaagggctgttcctgggcgggaactatgtcgctggtgttgcgttagggcgttgtgtggaaggcgcgtatgaaaccgccattgaagttaacaacttcatgagccgctacgcgtataaataa

SEQ ID NO: 4 - protein, 537 aa, synthetic construct
  source 1..537: note = Arabidopsis thaliana PPO1 G64A
MELSLLRPTTQSLLPSFSKPNLRLNVYKPLRLRCSVAGGPTVGSSKIEGGGGTTITTDCVIVGAGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 5 - protein, 537 aa, synthetic construct
  source 1..537: note = Arabidopsis thaliana PPO1 V88I
MELSLLRPTTQSLLPSFSKPNLRLNVYKPLRLRCSVAGGPTVGSSKIEGGGGTTITTDCVIVGGGISGLCIAQALATKHPDAAPNLIITEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 6 - protein, 537 aa, synthetic construct
  source 1..537: note = Arabidopsis thaliana PPO1 E111D
MELSLLRPTTQSLLPSFSKPNLRLNVYKPLRLRCSVAGGPTVGSSKIEGGGGTTITTDCVIVGGGISGLCIAQALATKHPDAAPNLIVTEAKDRVGGNIITREENGFLWEDGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 7 - protein, 537 aa, synthetic construct
  source 1..537: note = Arabidopsis thaliana PPO1 E111F
MELSLLRPTTQSLLPSFSKPNLRLNVYKPLRLRCSVAGGPTVGSSKIEGGGGTTITTDCVIVGGGISGLCIAQALATKHPDAAPNLIVTEAKDRVGGNIITREENGFLWEFGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 8 - protein, 537 aa, synthetic construct
  source 1..537: note = Arabidopsis thaliana PPO1 V127I
MELSLLRPTTQSLLPSFSKPNLRLNVYKPLRLRCSVAGGPTVGSSKIEGGGGTTITTDCVIVGGGISGLCIAQALATKHPDAAPNLIVTEAKDRVGGNIITREENGFLWEEGPNSFQPSDPMLTMVIDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 9 - protein, 537 aa, synthetic construct
  source 1..537: note = Arabidopsis thaliana PPO1 V127L
MELSLLRPTTQSLLPSFSKPNLRLNVYKPLRLRCSVAGGPTVGSSKIEGGGGTTITTDCVIVGGGISGLCIAQALATKHPDAAPNLIVTEAKDRVGGNIITREENGFLWEEGPNSFQPSDPMLTMVLDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 10 - protein, 537 aa, synthetic construct
  source 1..537: note = Arabidopsis thaliana PPO1 P154Y
MELSLLRPTTQSLLPSFSKPNLRLNVYKPLRLRCSVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRYVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 11 - protein, 537 aa, synthetic construct
  source 1..537: note = Arabidopsis thaliana PPO1 K173H
MELSLLRPTTQSLLPSFSKPNLRLNVYKPLRLRCSVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGHIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 12 - protein, 537 aa, synthetic construct
  source 1..537: note = Arabidopsis thaliana PPO1 A176L
MELSLLRPTTQSLLPSFSKPNLRLNVYKPLRLRCSVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRLGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 13 - protein, 537 aa, synthetic construct
  source 1..537: note = Arabidopsis thaliana PPO1 V198W
MELSLLRPTTQSLLPSFSKPNLRLNVYKPLRLRCSVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFWRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 14 - protein, 537 aa, synthetic construct
  source 1..537: note = Arabidopsis thaliana PPO1 V206G
MELSLLRPTTQSLLPSFSKPNLRLNVYKPLRLRCSVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEGFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 15 - protein, 537 aa, synthetic construct
  source 1..537: note = Arabidopsis thaliana PPO1 F207Y
MELSLLRPTTQSLLPSFSKPNLRLNVYKPLRLRCSVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVYERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 16 - protein, 537 aa, synthetic construct
  source 1..537: note = Arabidopsis thaliana PPO1 E212Y
MELSLLRPTTQSLLPSFSKPNLRLNVYKPLRLRCSVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIYPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 17 - protein, 537 aa, synthetic construct
  source 1..537: note = Arabidopsis thaliana PPO1 F214Y
MELSLLRPTTQSLLPSFSKPNLRLNVYKPLRLRCSVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPYCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 18 - protein, 537 aa, synthetic construct
  source 1..537: note = Arabidopsis thaliana PPO1 V218K
MELSLLRPTTQSLLPSFSKPNLRLNVYKPLRLRCSVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGKYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 19 - protein, 537 aa, synthetic construct
  source 1..537: note = Arabidopsis thaliana PPO1 Y219S
MELSLLRPTTQSLLPSFSKPNLRLNVYKPLRLRCSVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVSAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 20 - protein, 537 aa, synthetic construct
  source 1..537: note = Arabidopsis thaliana PPO1 A220C
MELSLLRPTTQSLLPSFSKPNLRLNVYKPLRLRCSVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYCGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 21 - protein, 537 aa, synthetic construct
  source 1..537: note = Arabidopsis thaliana PPO1 P271C
MELSLLRPTTQSLLPSFSKPNLRLNVYKPLRLRCSVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKCQGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 22 - protein, 537 aa, synthetic construct
  source 1..537: note = Arabidopsis thaliana PPO1 Q272H
MELSLLRPTTQSLLPSFSKPNLRLNVYKPLRLRCSVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPHGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 23 - protein, 537 aa, synthetic construct
  source 1..537: note = Arabidopsis thaliana PPO1 Q272I
MELSLLRPTTQSLLPSFSKPNLRLNVYKPLRLRCSVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPIGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 24 - protein, 537 aa, synthetic construct
  source 1..537: note = Arabidopsis thaliana PPO1 Q272T
MELSLLRPTTQSLLPSFSKPNLRLNVYKPLRLRCSVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPTGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 25 - protein, 537 aa, synthetic construct
  source 1..537: note = Arabidopsis thaliana PPO1 K281C
MELSLLRPTTQSLLPSFSKPNLRLNVYKPLRLRCSVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRCGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 26 - protein, 537 aa, synthetic construct
  source 1..537: note = Arabidopsis thaliana PPO1 K281E
MELSLLRPTTQSLLPSFSKPNLRLNVYKPLRLRCSVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFREGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 27 - protein, 537 aa, synthetic construct
  source 1..537: note = Arabidopsis thaliana PPO1 K281N
MELSLLRPTTQSLLPSFSKPNLRLNVYKPLRLRCSVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRNGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 28 - protein, 537 aa, synthetic construct
  source 1..537: note = Arabidopsis thaliana PPO1 R284G
MELSLLRPTTQSLLPSFSKPNLRLNVYKPLRLRCSVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLGMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 29 - protein, 537 aa, synthetic construct
  source 1..537: note = Arabidopsis thaliana PPO1 S305L
MELSLLRPTTQSLLPSFSKPNLRLNVYKPLRLRCSVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLLGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 30 - protein, 537 aa, synthetic construct
  source 1..537: note = Arabidopsis thaliana PPO1 L360C
MELSLLRPTTQSLLPSFSKPNLRLNVYKPLRLRCSVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKCYYPPVAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 31 - protein, 537 aa, synthetic construct
  source 1..537: note = Arabidopsis thaliana PPO1 Y361D
MELSLLRPTTQSLLPSFSKPNLRLNVYKPLRLRCSVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLDYPPVAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 32 - protein, 537 aa, synthetic construct
  source 1..537: note = Arabidopsis thaliana PPO1 Y361F
MELSLLRPTTQSLLPSFSKPNLRLNVYKPLRLRCSVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLFYPPVAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 33 - protein, 537 aa, synthetic construct
  source 1..537: note = Arabidopsis thaliana PPO1 Y361P
MELSLLRPTTQSLLPSFSKPNLRLNVYKPLRLRCSVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLPYPPVAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 34 - protein, 537 aa, synthetic construct
  source 1..537: note = Arabidopsis thaliana PPO1 Y361Q
MELSLLRPTTQSLLPSFSKPNLRLNVYKPLRLRCSVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLQYPPVAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 35 - protein, 537 aa, synthetic construct
  source 1..537: note = Arabidopsis thaliana PPO1 Y361R
MELSLLRPTTQSLLPSFSKPNLRLNVYKPLRLRCSVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYRPVAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 36 - protein, 537 aa, synthetic construct
  source 1..537: note = Arabidopsis thaliana PPO1 Y361T
MELSLLRPTTQSLLPSFSKPNLRLNVYKPLRLRCSVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLTYPPVAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 37 - protein, 537 aa, synthetic construct
  source 1..537: note = Arabidopsis thaliana PPO1 Y362F
MELSLLRPTTQSLLPSFSKPNLRLNVYKPLRLRCSVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYFPPVAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 38 - protein, 537 aa, synthetic construct
  source 1..537: note = Arabidopsis thaliana PPO1 Y365L
MELSLLRPTTQSLLPSFSKPNLRLNVYKPLRLRCSVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPLAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 39 - protein, 537 aa, synthetic construct
  source 1..537: note = Arabidopsis thaliana PPO1 Y365M
MELSLLRPTTQSLLPSFSKPNLRLNVYKPLRLRCSVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPMAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 40 - protein, 537 aa, synthetic construct
  source 1..537: note = Arabidopsis thaliana PPO1 G404A
MELSLLRPTTQSLLPSFSKPNLRLNVYKPLRLRCSVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLATIYSSSLFPNRAPPGRILLLNYIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 41 - protein, 537 aa, synthetic construct
  source 1..537: note = Arabidopsis thaliana PPO1 P413G
MELSLLRPTTQSLLPSFSKPNLRLNVYKPLRLRCSVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFGNRAPPGRILLLNYIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 42 - protein, 537 aa, synthetic construct
  source 1..537: note = Arabidopsis thaliana PPO1 P418M
MELSLLRPTTQSLLPSFSKPNLRLNVYKPLRLRCSVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPMGRILLLNYIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 43 - protein, 537 aa, synthetic construct
  source 1..537: note = Arabidopsis thaliana PPO1 L424V
MELSLLRPTTQSLLPSFSKPNLRLNVYKPLRLRCSVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLVNYIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 44 - protein, 537 aa, synthetic construct
  source 1..537: note = Arabidopsis thaliana PPO1 Y426C
MELSLLRPTTQSLLPSFSKPNLRLNVYKPLRLRCSVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNCIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 45 - protein, 537 aa, synthetic construct
  source 1..537: note = Arabidopsis thaliana PPO1 Y426L
MELSLLRPTTQSLLPSFSKPNLRLNVYKPLRLRCSVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNLIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 46 - protein, 537 aa, synthetic construct
  source 1..537: note = Arabidopsis thaliana PPO1 Y426M
MELSLLRPTTQSLLPSFSKPNLRLNVYKPLRLRCSVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNMIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 47 - protein, 537 aa, synthetic construct
  source 1..537: note = Arabidopsis thaliana PPO1 Y426T
MELSLLRPTTQSLLPSFSKPNLRLNVYKPLRLRCSVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNTIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 48 - protein, 537 aa, synthetic construct
  source 1..537: note = Arabidopsis thaliana PPO1 Y426V
MELSLLRPTTQSLLPSFSKPNLRLNVYKPLRLRCSVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNVIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 49 - protein, 537 aa, synthetic construct
  source 1..537: note = Arabidopsis thaliana PPO1 T431R
MELSLLRPTTQSLLPSFSKPNLRLNVYKPLRLRCSVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNYIGGSRNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 50 - protein, 537 aa, synthetic construct
  source 1..537: note = Arabidopsis thaliana PPO1 V447C
MELSLLRPTTQSLLPSFSKPNLRLNVYKPLRLRCSVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNYIGGSTNTGILSKSEGELVEACDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 51 - protein, 537 aa, synthetic construct
  source 1..537: note = Arabidopsis thaliana PPO1 V447I
MELSLLRPTTQSLLPSFSKPNLRLNVYKPLRLRCSVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNYIGGSTNTGILSKSEGELVEAIDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 52 - protein, 537 aa, synthetic construct
  source 1..537: note = Arabidopsis thaliana PPO1 D448H
MELSLLRPTTQSLLPSFSKPNLRLNVYKPLRLRCSVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNYIGGSTNTGILSKSEGELVEAVHRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 53 - protein, 537 aa, synthetic construct
  source 1..537: note = Arabidopsis thaliana PPO1 D448L
MELSLLRPTTQSLLPSFSKPNLRLNVYKPLRLRCSVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNYIGGSTNTGILSKSEGELVEAVLRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 54 - protein, 537 aa, synthetic construct
  source 1..537: note = Arabidopsis thaliana PPO1 D448N
MELSLLRPTTQSLLPSFSKPNLRLNVYKPLRLRCSVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNYIGGSTNTGILSKSEGELVEAVNRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 55 - protein, 537 aa, synthetic construct
  source 1..537: note = Arabidopsis thaliana PPO1 R449K
MELSLLRPTTQSLLPSFSKPNLRLNVYKPLRLRCSVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNYIGGSTNTGILSKSEGELVEAVDKDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 56 - protein, 537 aa, synthetic construct
  source 1..537: note = Arabidopsis thaliana PPO1 T461Q
MELSLLRPTTQSLLPSFSKPNLRLNVYKPLRLRCSVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNYIGGSTNTGILSKSEGELVEAVDRDLRKMLIKPNSQDPLKLGVRVWPQAIPQFLVGHFDILDTAKSSLTSSGYEGLFLGGNYVAGVALGRCVEGAYETAIEVNNFMSRYAYK

SEQ ID NO: 57 - protein, 537 aa, synthetic construct
  source 1..537: note = Arabidopsis thaliana PPO1 F478R
MELSLLRPTTQSLLPSFSKPNLRLNVYKPLRLRCSVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQRLVGHFDILDTAKSSLTSSGYEGLFLGGNYVAGVALGRCVEGAYETAIEVNNFMSRYAYK

SEQ ID NO: 58 - protein, 537 aa, synthetic construct
  source 1..537: note = Arabidopsis thaliana PPO1 L479M
MELSLLRPTTQSLLPSFSKPNLRLNVYKPLRLRCSVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFMVGHFDILDTAKSSLTSSGYEGLFLGGNYVAGVALGRCVEGAYETAIEVNNFMSRYAYK

SEQ ID NO: 59 - protein, 537 aa, synthetic construct
  source 1..537: note = Arabidopsis thaliana PPO1 L479N
MELSLLRPTTQSLLPSFSKPNLRLNVYKPLRLRCSVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFNVGHFDILDTAKSSLTSSGYEGLFLGGNYVAGVALGRCVEGAYETAIEVNNFMSRYAYK

SEQ ID NO: 60 - protein, 537 aa, synthetic construct
  source 1..537: note = Arabidopsis thaliana PPO1 G510N
MELSLLRPTTQSLLPSFSKPNLRLNVYKPLRLRCSVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFLVGHFDILDTAKSSLTSSGYEGLFLGGNYVANVALGRCVEGAYETAIEVNNFMSRYAYK

SEQ ID NO: 61 - protein, 537 aa, synthetic construct
  source 1..537: note = Arabidopsis thaliana PPO1 V511L
MELSLLRPTTQSLLPSFSKPNLRLNVYKPLRLRCSVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFLVGHFDILDTAKSSLTSSGYEGLFLGGNYVAGLALGRCVEGAYETAIEVNNFMSRYAYK

SEQ ID NO: 62 - protein, 537 aa, synthetic construct
  source 1..537: note = Arabidopsis thaliana PPO1 V511N
MELSLLRPTTQSLLPSFSKPNLRLNVYKPLRLRCSVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFLVGHFDILDTAKSSLTSSGYEGLFLGGNYVAGNALGRCVEGAYETAIEVNNFMSRYAYK

SEQ ID NO: 63 - protein, 537 aa, synthetic construct
  source 1..537: note = Arabidopsis thaliana PPO1 C516Q
MELSLLRPTTQSLLPSFSKPNLRLNVYKPLRLRCSVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFLVGHFDILDTAKSSLTSSGYEGLFLGGNYVAGVALGRQVEGAYETAIEVNNFMSRYAYK

SEQ ID NO: 64 - protein, 535 aa, synthetic construct
  source 1..535: note = Setaria italica PPO1 G62A
MVAAAMATAPSAGVPPLRGTRGPARFRIRGVSVRCAAVAGGAAEAPASAGARVSADCVVVGAGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 65 - protein, 535 aa, synthetic construct
  source 1..535: note = Setaria italica PPO1 V84I
MVAAAMATAPSAGVPPLRGTRGPARFRIRGVSVRCAAVAGGAAEAPASAGARVSADCVVVGGGISGLCTAQALATKHGVGDVLITEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 66 - protein, 535 aa, synthetic construct
  source 1..535: note = Setaria italica PPO1 E109D
MVAAAMATAPSAGVPPLRGTRGPARFRIRGVSVRCAAVAGGAAEAPASAGARVSADCVVVGGGISGLCTAQALATKHGVGDVLVTEARARPGGNITTVERPDEGYLWEDGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 67 - protein, 535 aa, synthetic construct
  source 1..535: note = Setaria italica PPO1 E109F
MVAAAMATAPSAGVPPLRGTRGPARFRIRGVSVRCAAVAGGAAEAPASAGARVSADCVVVGGGISGLCTAQALATKHGVGDVLVTEARARPGGNITTVERPDEGYLWEFGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 68 - protein, 535 aa, synthetic construct
  source 1..535: note = Setaria italica PPO1 V125I
MVAAAMATAPSAGVPPLRGTRGPARFRIRGVSVRCAAVAGGAAEAPASAGARVSADCVVVGGGISGLCTAQALATKHGVGDVLVTEARARPGGNITTVERPDEGYLWEEGPNSFQPSDPVLTMAIDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 69 - protein, 535 aa, synthetic construct
  source 1..535: note = Setaria italica PPO1 V125L
MVAAAMATAPSAGVPPLRGTRGPARFRIRGVSVRCAAVAGGAAEAPASAGARVSADCVVVGGGISGLCTAQALATKHGVGDVLVTEARARPGGNITTVERPDEGYLWEEGPNSFQPSDPVLTMALDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 70 - protein, 535 aa, synthetic construct
  source 1..535: note = Setaria italica PPO1 P152Y
MVAAAMATAPSAGVPPLRGTRGPARFRIRGVSVRCAAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRYVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 71 - protein, 535 aa, synthetic construct
  source 1..535: note = Setaria italica PPO1 K171H
MVAAAMATAPSAGVPPLRGTRGPARFRIRGVSVRCAAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGHLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 72 - protein, 535 aa, synthetic construct
  source 1..535: note = Setaria italica PPO1 A174L
MVAAAMATAPSAGVPPLRGTRGPARFRIRGVSVRCAAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRLGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 73 - protein, 535 aa, synthetic construct
  source 1..535: note = Setaria italica PPO1 V196W
MVAAAMATAPSAGVPPLRGTRGPARFRIRGVSVRCAAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFWRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 74 - protein, 535 aa, synthetic construct
  source 1..535: note = Setaria italica PPO1 V204G
MVAAAMATAPSAGVPPLRGTRGPARFRIRGVSVRCAAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEGFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 75 - protein, 535 aa, synthetic construct
  source 1..535: note = Setaria italica PPO1 F205Y
MVAAAMATAPSAGVPPLRGTRGPARFRIRGVSVRCAAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVYERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 76 - protein, 535 aa, synthetic construct
  source 1..535: note = Setaria italica PPO1 E210Y
MVAAAMATAPSAGVPPLRGTRGPARFRIRGVSVRCAAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIYPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 77 - protein, 535 aa, synthetic construct
  source 1..535: note = Setaria italica PPO1 F212Y
MVAAAMATAPSAGVPPLRGTRGPARFRIRGVSVRCAAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPYCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 78 - protein, 535 aa, synthetic construct
  source 1..535: note = Setaria italica PPO1 V216K
MVAAAMATAPSAGVPPLRGTRGPARFRIRGVSVRCAAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGKYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 79 - protein, 535 aa, synthetic construct
  source 1..535: note = Setaria italica PPO1 Y217S
MVAAAMATAPSAGVPPLRGTRGPARFRIRGVSVRCAAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVSAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 80 - protein, 535 aa, synthetic construct
  source 1..535: note = Setaria italica PPO1 A218C
MVAAAMATAPSAGVPPLRGTRGPARFRIRGVSVRCAAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYCGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 81 - protein, 535 aa, synthetic construct
  source 1..535: note = Setaria italica PPO1 P269C
MVAAAMATAPSAGVPPLRGTRGPARFRIRGVSVRCAAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTCKGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 82 - protein, 535 aa, synthetic construct
  source 1..535: note = Setaria italica PPO1 K270H
MVAAAMATAPSAGVPPLRGTRGPARFRIRGVSVRCAAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPHGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 83 - protein, 535 aa, synthetic construct
  source 1..535: note = Setaria italica PPO1 K270I
MVAAAMATAPSAGVPPLRGTRGPARFRIRGVSVRCAAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPIGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 84 - protein, 535 aa, synthetic construct
  source 1..535: note = Setaria italica PPO1 K270T
MVAAAMATAPSAGVPPLRGTRGPARFRIRGVSVRCAAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPTGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 85 - protein, 535 aa, synthetic construct
  source 1..535: note = Setaria italica PPO1 K279C
MVAAAMATAPSAGVPPLRGTRGPARFRIRGVSVRCAAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRCGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 86 - protein, 535 aa, synthetic construct
  source 1..535: note = Setaria italica PPO1 K279E
MVAAAMATAPSAGVPPLRGTRGPARFRIRGVSVRCAAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFREGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 87 - protein, 535 aa, synthetic construct
  source 1..535: note = Setaria italica PPO1 K279N
MVAAAMATAPSAGVPPLRGTRGPARFRIRGVSVRCAAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRNGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 88 - protein, 535 aa, synthetic construct
  source 1..535: note = Setaria italica PPO1 A282G
MVAAAMATAPSAGVPPLRGTRGPARFRIRGVSVRCAAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLGMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 89 - protein, 535 aa, synthetic construct
  source 1..535: note = Setaria italica PPO1 T303L
MVAAAMATAPSAGVPPLRGTRGPARFRIRGVSVRCAAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLLSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 90 - protein, 535 aa, synthetic construct
  source 1..535: note = Setaria italica PPO1 F358C
MVAAAMATAPSAGVPPLRGTRGPARFRIRGVSVRCAAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRCYYPPVAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 91 - protein, 535 aa, synthetic construct
  source 1..535: note = Setaria italica PPO1 Y359D
MVAAAMATAPSAGVPPLRGTRGPARFRIRGVSVRCAAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFDYPPVAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 92 - protein, 535 aa, synthetic construct
  source 1..535: note = Setaria italica PPO1 Y359F
MVAAAMATAPSAGVPPLRGTRGPARFRIRGVSVRCAAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFFYPPVAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 93 - protein, 535 aa, synthetic construct
  source 1..535: note = Setaria italica PPO1 Y359P
MVAAAMATAPSAGVPPLRGTRGPARFRIRGVSVRCAAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFPYPPVAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 94 - protein, 535 aa, synthetic construct
  source 1..535: note = Setaria italica PPO1 Y359Q
MVAAAMATAPSAGVPPLRGTRGPARFRIRGVSVRCAAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFQYPPVAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 95 - protein, 535 aa, synthetic construct
  source 1..535: note = Setaria italica PPO1 Y359R
MVAAAMATAPSAGVPPLRGTRGPARFRIRGVSVRCAAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFRYPPVAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 96 - protein, 535 aa, synthetic construct
  source 1..535: note = Setaria italica PPO1 Y359T
MVAAAMATAPSAGVPPLRGTRGPARFRIRGVSVRCAAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFTYPPVAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 97 - protein, 535 aa, synthetic construct
  source 1..535: note = Setaria italica PPO1 Y360F
MVAAAMATAPSAGVPPLRGTRGPARFRIRGVSVRCAAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYFPPVAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 98 - protein, 535 aa, synthetic construct
  source 1..535: note = Setaria italica PPO1 V363L
MVAAAMATAPSAGVPPLRGTRGPARFRIRGVSVRCAAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYYPPLAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 99 - protein, 535 aa, synthetic construct
  source 1..535: note = Setaria italica PPO1 V363M
MVAAAMATAPSAGVPPLRGTRGPARFRIRGVSVRCAAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYYPPMAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 100 - protein, 535 aa, synthetic construct
  source 1..535: note = Setaria italica PPO1 G402A
MVAAAMATAPSAGVPPLRGTRGPARFRIRGVSVRCAAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLATIYSSSLFPNRAPAGRVLLLNYIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 101 - protein, 535 aa, synthetic construct
  source 1..535: note = Setaria italica PPO1 P411G
MVAAAMATAPSAGVPPLRGTRGPARFRIRGVSVRCAAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFGNRAPAGRVLLLNYIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 102 - protein, 535 aa, synthetic construct
  source 1..535: note = Setaria italica PPO1 A416M
MVAAAMATAPSAGVPPLRGTRGPARFRIRGVSVRCAAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPMGRVLLLNYIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 103 - protein, 535 aa, synthetic construct
  source 1..535: note = Setaria italica PPO1 L422V
MVAAAMATAPSAGVPPLRGTRGPARFRIRGVSVRCAAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLVNYIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 104 - protein, 535 aa, synthetic construct
  source 1..535: note = Setaria italica PPO1 Y424C
MVAAAMATAPSAGVPPLRGTRGPARFRIRGVSVRCAAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNCIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 105 - protein, 535 aa, synthetic construct
  source 1..535: note = Setaria italica PPO1 Y424L
MVAAAMATAPSAGVPPLRGTRGPARFRIRGVSVRCAAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNLIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 106 - protein, 535 aa, synthetic construct
  source 1..535: note = Setaria italica PPO1 Y424M
MVAAAMATAPSAGVPPLRGTRGPARFRIRGVSVRCAAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNMIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 107 - protein, 535 aa, synthetic construct
  source 1..535: note = Setaria italica PPO1 Y424T
MVAAAMATAPSAGVPPLRGTRGPARFRIRGVSVRCAAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNTIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 108 - protein, 535 aa, synthetic construct
  source 1..535: note = Setaria italica PPO1 Y424V
MVAAAMATAPSAGVPPLRGTRGPARFRIRGVSVRCAAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNVIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 109 - protein, 535 aa, synthetic construct
  source 1..535: note = Setaria italica PPO1 T429R
MVAAAMATAPSAGVPPLRGTRGPARFRIRGVSVRCAAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNYIGGARNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 110 - protein, 535 aa, synthetic construct
  source 1..535: note = Setaria italica PPO1 V445C
MVAAAMATAPSAGVPPLRGTRGPARFRIRGVSVRCAAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNYIGGATNTGIVSKSASELVEACDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 111 - protein, 535 aa, synthetic construct
  source 1..535: note = Setaria italica PPO1 V445I
MVAAAMATAPSAGVPPLRGTRGPARFRIRGVSVRCAAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNYIGGATNTGIVSKSASELVEAIDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 112 - protein, 535 aa, synthetic construct
  source 1..535: note = Setaria italica PPO1 D446H
MVAAAMATAPSAGVPPLRGTRGPARFRIRGVSVRCAAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNYIGGATNTGIVSKSASELVEAVHRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 113 - protein, 535 aa, synthetic construct
  source 1..535: note = Setaria italica PPO1 D446L
MVAAAMATAPSAGVPPLRGTRGPARFRIRGVSVRCAAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNYIGGATNTGIVSKSASELVEAVLRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 114 - protein, 535 aa, synthetic construct
  source 1..535: note = Setaria italica PPO1 D446N
MVAAAMATAPSAGVPPLRGTRGPARFRIRGVSVRCAAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNYIGGATNTGIVSKSASELVEAVNRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 115 - protein, 535 aa, synthetic construct
  source 1..535: note = Setaria italica PPO1 R447K
MVAAAMATAPSAGVPPLRGTRGPARFRIRGVSVRCAAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNYIGGATNTGIVSKSASELVEAVDKDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 116 - protein, 535 aa, synthetic construct
  source 1..535: note = Setaria italica PPO1 V459Q
MVAAAMATAPSAGVPPLRGTRGPARFRIRGVSVRCAAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNYIGGATNTGIVSKSASELVEAVDRDLRKMLINPSAQDPLVLGVRVWPQAIPQFLVGHLDLLEAAKSSLDRGGYDGLFLGGNYVAGVALGRCVEGAYESASQISDFLTKYAYK

SEQ ID NO: 117 - protein, 535 aa, synthetic construct
  source 1..535: note = Setaria italica PPO1 F476R
MVAAAMATAPSAGVPPLRGTRGPARFRIRGVSVRCAAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQRLVGHLDLLEAAKSSLDRGGYDGLFLGGNYVAGVALGRCVEGAYESASQISDFLTKYAYK

SEQ ID NO: 118 - protein, 535 aa, synthetic construct
  source 1..535: note = Setaria italica PPO1 L477M
MVAAAMATAPSAGVPPLRGTRGPARFRIRGVSVRCAAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQFMVGHLDLLEAAKSSLDRGGYDGLFLGGNYVAGVALGRCVEGAYESASQISDFLTKYAYK

SEQ ID NO: 119 - protein, 535 aa, synthetic construct
  source 1..535: note = Setaria italica PPO1 L477N
MVAAAMATAPSAGVPPLRGTRGPARFRIRGVSVRCAAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQFNVGHLDLLEAAKSSLDRGGYDGLFLGGNYVAGVALGRCVEGAYESASQISDFLTKYAYK

SEQ ID NO: 120 - protein, 535 aa, synthetic construct
  source 1..535: note = Setaria italica PPO1 G508N
MVAAAMATAPSAGVPPLRGTRGPARFRIRGVSVRCAAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQFLVGHLDLLEAAKSSLDRGGYDGLFLGGNYVANVALGRCVEGAYESASQISDFLTKYAYK

SEQ ID NO: 121 - protein, 535 aa, synthetic construct
  source 1..535: note = Setaria italica PPO1 V509L
MVAAAMATAPSAGVPPLRGTRGPARFRIRGVSVRCAAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQFLVGHLDLLEAAKSSLDRGGYDGLFLGGNYVAGLALGRCVEGAYESASQISDFLTKYAYK

SEQ ID NO: 122 - protein, 535 aa, synthetic construct
  source 1..535: note = Setaria italica PPO1 V509N
MVAAAMATAPSAGVPPLRGTRGPARFRIRGVSVRCAAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQFLVGHLDLLEAAKSSLDRGGYDGLFLGGNYVAGNALGRCVEGAYESASQISDFLTKYAYK

SEQ ID NO: 123 - protein, 535 aa, synthetic construct
  source 1..535: note = Setaria italica PPO1 C514Q
MVAAAMATAPSAGVPPLRGTRGPARFRIRGVSVRCAAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQFLVGHLDLLEAAKSSLDRGGYDGLFLGGNYVAGVALGRQVEGAYESASQISDFLTKYAYK

SEQ ID NO: 124 - protein, 537 aa, synthetic construct
  source 1..537: note = Arabidopsis thaliana PPO1 S305L Y426M
MELSLLRPTTQSLLPSFSKPNLRLNVYKPLRLRCSVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLLGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNMIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 125 - protein, 537 aa, synthetic construct
  source 1..537: note = Arabidopsis thaliana PPO1 S305L Y426V
MELSLLRPTTQSLLPSFSKPNLRLNVYKPLRLRCSVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLLGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNVIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 126 - protein, 537 aa, synthetic construct
  source 1..537: note = Arabidopsis thaliana PPO1 V365L Y426V L479N
MELSLLRPTTQSLLPSFSKPNLRLNVYKPLRLRCSVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPLAAVSISYPKEAIRTECLIDGELKGFGQLHPRTQGVETLGTIYSSSLFPNRAPPGRILLLNVIGGSTNTGILSKSEGELVEAVDRDLRKMLIKPNSTDPLKLGVRVWPQAIPQFNVGHFDILDTAKSSLTSSGYEGLFLGGNYVAGVALGRCVEGAYETAIEVNNFMSRYAYK

SEQ ID NO: 127 - protein, 537 aa, synthetic construct
  source 1..537: note = Arabidopsis thaliana PPO1 S305L G404A Y426V T431R L479N
MELSLLRPTTQSLLPSFSKPNLRLNVYKPLRLRCSVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLLGITKLESGGYNLTYETPDGLVSVQSKSVVMTVPSHVASGLLRPLSESAANALSKLYYPPVAAVSISYPKEAIRTECLIDGELKGFGQLHPRTQGVETLATIYSSSLFPNRAPPGRILLLNVIGGSRNTGILSKSEGELVEAVDRDLRKMLIKPNSTDPLKLGVRVWPQAIPQFNVGHFDILDTAKSSLTSSGYEGLFLGGNYVAGVALGRCVEGAYETAIEVNNFMSRYAYK

SEQ ID NO: 128 - protein, 537 aa, synthetic construct
  source 1..537: note = Arabidopsis thaliana PPO1 S305L V365M
MELSLLRPTTQSLLPSFSKPNLRLNVYKPLRLRCSVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLLGITKLESGGYNLTYETPDGLVSVQSKSVVMTVPSHVASGLLRPLSESAANALSKLYYPPMAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 129 - protein, 537 aa, synthetic construct
  source 1..537: note = Arabidopsis thaliana PPO1 S305L Y362F G404A
MELSLLRPTTQSLLPSFSKPNLRLNVYKPLRLRCSVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLLGITKLESGGYNLTYETPDGLVSVQSKSVVMTVPSHVASGLLRPLSESAANALSKLYFPPVAAVSISYPKEAIRTECLIDGELKGFGQLHPRTQGVETLATIYSSSLFPNRAPPGRILLLNYIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 130 - protein, 537 aa, synthetic construct
  source 1..537: note = Arabidopsis thaliana PPO1 S305L Y361T V365L T431R L479M
MELSLLRPTTQSLLPSFSKPNLRLNVYKPLRLRCSVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLLGITKLESGGYNLTYETPDGLVSVQSKSVVMTVPSHVASGLLRPLSESAANALSKLTYPPLAAVSISYPKEAIRTECLIDGELKGFGQLHPRTQGVETLGTIYSSSLFPNRAPPGRILLLNYIGGSRNTGILSKSEGELVEAVDRDLRKMLIKPNSTDPLKLGVRVWPQAIPQFMVGHFDILDTAKSSLTSSGYEGLFLGGNYVAGVALGRCVEGAYETAIEVNNFMSRYAYK

SEQ ID NO: 131 - protein, 537 aa, synthetic construct
  source 1..537: note = Arabidopsis thaliana PPO1 S305L Y426V L479M
MELSLLRPTTQSLLPSFSKPNLRLNVYKPLRLRCSVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLLGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNVIGGSTNTGILSKSEGELVEAVDRDLRKMLIKPNSTDPLKLGVRVWPQAIPQFMVGHFDILDTAKSSLTSSGYEGLFLGGNYVAGVALGRCVEGAYETAIEVNNFMSRYAYK

SEQ ID NO: 132 - protein, 537 aa, unidentified
  source 1..537: note = Arabidopsis thaliana PPO1 Y426M T461Q L479M
MELSLLRPTTQSLLPSFSKPNLRLNVYKPLRLRCSVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNMIGGSTNTGILSKSEGELVEAVDRDLRKMLIKPNSQDPLKLGVRVWPQAIPQFMVGHFDILDTAKSSLTSSGYEGLFLGGNYVAGVALGRCVEGAYETAIEVNNFMSRYAYK

SEQ ID NO: 133 - protein, 537 aa, synthetic construct
  source 1..537: note = Arabidopsis thaliana PPO1 S305L Y361D V365M
MELSLLRPTTQSLLPSFSKPNLRLNVYKPLRLRCSVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLLGITKLESGGYNLTYETPDGLVSVQSKSVVMTVPSHVASGLLRPLSESAANALSKLDYPPMAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 134 - protein, 537 aa, synthetic construct
  source 1..537: note = Arabidopsis thaliana PPO1 S305L Y361C Y362F Y426M L479M
MELSLLRPTTQSLLPSFSKPNLRLNVYKPLRLRCSVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLLGITKLESGGYNLTYETPDGLVSVQSKSVVMTVPSHVASGLLRPLSESAANALSKLCFPPVAAVSISYPKEAIRTECLIDGELKGFGQLHPRTQGVETLGTIYSSSLFPNRAPPGRILLLNMIGGSTNTGILSKSEGELVEAVDRDLRKMLIKPNSTDPLKLGVRVWPQAIPQFMVGHFDILDTAKSSLTSSGYEGLFLGGNYVAGVALGRCVEGAYETAIEVNNFMSRYAYK

SEQ ID NO: 135 - protein, 537 aa, synthetic construct
  source 1..537: note = Arabidopsis thaliana PPO1 Y361D V365L
MELSLLRPTTQSLLPSFSKPNLRLNVYKPLRLRCSVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLDYPPLAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 136 - protein, 537 aa, synthetic construct
  source 1..537: note = Arabidopsis thaliana PPO1 V365L G404A L479N
MELSLLRPTTQSLLPSFSKPNLRLNVYKPLRLRCSVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPLAAVSISYPKEAIRTECLIDGELKGFGQLHPRTQGVETLATIYSSSLFPNRAPPGRILLLNYIGGSTNTGILSKSEGELVEAVDRDLRKMLIKPNSTDPLKLGVRVWPQAIPQFNVGHFDILDTAKSSLTSSGYEGLFLGGNYVAGVALGRCVEGAYETAIEVNNFMSRYAYK

SEQ ID NO: 137 - protein, 537 aa, synthetic construct
  source 1..537: note = Arabidopsis thaliana PPO1 S305L G404A L479N
MELSLLRPTTQSLLPSFSKPNLRLNVYKPLRLRCSVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLLGITKLESGGYNLTYETPDGLVSVQSKSVVMTVPSHVASGLLRPLSESAANALSKLYYPPVAAVSISYPKEAIRTECLIDGELKGFGQLHPRTQGVETLATIYSSSLFPNRAPPGRILLLNYIGGSTNTGILSKSEGELVEAVDRDLRKMLIKPNSTDPLKLGVRVWPQAIPQFNVGHFDILDTAKSSLTSSGYEGLFLGGNYVAGVALGRCVEGAYETAIEVNNFMSRYAYK

SEQ ID NO: 138 - protein, 535 aa, synthetic construct
  source 1..535: note = Setaria italica PPO1 T303L Y424M
MVAAAMATAPSAGVPPLRGTRGPARFRIRGVSVRCAAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLLSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNMIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 139 - protein, 535 aa, synthetic construct
  source 1..535: note = Setaria italica PPO1 T303L Y424V
MVAAAMATAPSAGVPPLRGTRGPARFRIRGVSVRCAAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLLSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNVIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 140 - protein, 535 aa, synthetic construct
  source 1..535: note = Setaria italica PPO1 V363L Y424V L477N
MVAAAMATAPSAGVPPLRGTRGPARFRIRGVSVRCAAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYYPPLAAVTISYPKEAIRKECLIDGELQGFGQLHPRSQGVETLGTIYSSSLFPNRAPAGRVLLLNVIGGATNTGIVSKSASELVEAVDRDLRKMLINPSAVDPLVLGVRVWPQAIPQFNVGHLDLLEAAKSSLDRGGYDGLFLGGNYVAGVALGRCVEGAYESASQISDFLTKYAYK

SEQ ID NO: 141 - protein, 535 aa, synthetic construct
  source 1..535: note = Setaria italica PPO1 T303L G402A Y424V T429R L477N
MVAAAMATAPSAGVPPLRGTRGPARFRIRGVSVRCAAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLLSITKSDGMGYVLVYETPEGVVSVQAKSVIMTIPSYVASDILRPLSSDAADALSRFYYPPVAAVTISYPKEAIRKECLIDGELQGFGQLHPRSQGVETLATIYSSSLFPNRAPAGRVLLLNVIGGARNTGIVSKSASELVEAVDRDLRKMLINPSAVDPLVLGVRVWPQAIPQFNVGHLDLLEAAKSSLDRGGYDGLFLGGNYVAGVALGRCVEGAYESASQISDFLTKYAYK

SEQ ID NO: 142 - protein, 535 aa, synthetic construct
  source 1..535: note = Setaria italica PPO1 T303L V363M
MVAAAMATAPSAGVPPLRGTRGPARFRIRGVSVRCAAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLLSITKSDGMGYVLVYETPEGVVSVQAKSVIMTIPSYVASDILRPLSSDAADALSRFYYPPMAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 143 - protein, 535 aa, synthetic construct
  source 1..535: note = Setaria italica PPO1 T303L Y360F G402A
MVAAAMATAPSAGVPPLRGTRGPARFRIRGVSVRCAAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLLSITKSDGMGYVLVYETPEGVVSVQAKSVIMTIPSYVASDILRPLSSDAADALSRFYFPPVAAVTISYPKEAIRKECLIDGELQGFGQLHPRSQGVETLATIYSSSLFPNRAPAGRVLLLNYIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 144 - protein, 535 aa, synthetic construct
  source 1..535: note = Setaria italica PPO1 T303L Y359T V363L T429R L477M
MVAAAMATAPSAGVPPLRGTRGPARFRIRGVSVRCAAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLLSITKSDGMGYVLVYETPEGVVSVQAKSVIMTIPSYVASDILRPLSSDAADALSRFTYPPLAAVTISYPKEAIRKECLIDGELQGFGQLHPRSQGVETLGTIYSSSLFPNRAPAGRVLLLNYIGGAANTGIVSKSASELVEAVDRDLRKMLINPSAVDPLVLGVRVWPQAIPQFMVGHLDLLEAAKSSLDRGGYDGLFLGGNYVAGVALGRCVEGAYESASQISDFLTKYAYK

SEQ ID NO: 145 - protein, 535 aa, synthetic construct
  source 1..535: note = Setaria italica PPO1 T303L Y424V L477M
MVAAAMATAPSAGVPPLRGTRGPARFRIRGVSVRCAAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLLSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNVIGGATNTGIVSKSASELVEAVDRDLRKMLINPSAVDPLVLGVRVWPQAIPQFMVGHLDLLEAAKSSLDRGGYDGLFLGGNYVAGVALGRCVEGAYESASQISDFLTKYAYK

SEQ ID NO: 146 - protein, 535 aa, synthetic construct
  source 1..535: note = Setaria italica PPO1 Y424M V459Q L477M
MVAAAMATAPSAGVPPLRGTRGPARFRIRGVSVRCAAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNMIGGATNTGIVSKSASELVEAVDRDLRKMLINPSAQDPLVLGVRVWPQAIPQFMVGHLDLLEAAKSSLDRGGYDGLFLGGNYVAGVALGRCVEGAYESASQISDFLTKYAYK

SEQ ID NO: 147 - protein, 535 aa, synthetic construct
  source 1..535: note = Setaria italica PPO1 T303L Y359D V363M
MVAAAMATAPSAGVPPLRGTRGPARFRIRGVSVRCAAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLLSITKSDGMGYVLVYETPEGVVSVQAKSVIMTIPSYVASDILRPLSSDAADALSRFDYPPMAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 148 - protein, 535 aa, synthetic construct
  source 1..535: note = Setaria italica PPO1 T303L Y359C Y360F Y424M L477M
MVAAAMATAPSAGVPPLRGTRGPARFRIRGVSVRCAAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLLSITKSDGMGYVLVYETPEGVVSVQAKSVIMTIPSYVASDILRPLSSDAADALSRFCFPPVAAVTISYPKEAIRKECLIDGELQGFGQLHPRSQGVETLGTIYSSSLFPNRAPAGRVLLLNMIGGATNTGIVSKSASELVEAVDRDLRKMLINPSAVDPLVLGVRVWPQAIPQFMVGHLDLLEAAKSSLDRGGYDGLFLGGNYVAGVALGRCVEGAYESASQISDFLTKYAYK

SEQ ID NO: 149 - protein, 535 aa, synthetic construct
  source 1..535: note = Setaria italica PPO1 Y359D V363L
MVAAAMATAPSAGVPPLRGTRGPARFRIRGVSVRCAAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFDYPPLAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 150 - protein, 535 aa, synthetic construct
  source 1..535: note = Setaria italica PPO1 V363L G402A L477N
MVAAAMATAPSAGVPPLRGTRGPARFRIRGVSVRCAAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYYPPLAAVTISYPKEAIRKECLIDGELQGFGQLHPRSQGVETLATIYSSSLFPNRAPAGRVLLLNYIGGATNTGIVSKSASELVEAVDRDLRKMLINPSAVDPLVLGVRVWPQAIPQFNVGHLDLLEAAKSSLDRGGYDGLFLGGNYVAGVALGRCVEGAYESASQISDFLTKYAYK

SEQ ID NO: 151 - protein, 535 aa, synthetic construct
  source 1..535: note = Setaria italica PPO1 T303L G402A L477N
MVAAAMATAPSAGVPPLRGTRGPARFRIRGVSVRCAAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLLSITKSDGMGYVLVYETPEGVVSVQAKSVIMTIPSYVASDILRPLSSDAADALSRFYYPPVAAVTISYPKEAIRKECLIDGELQGFGQLHPRSQGVETLATIYSSSLFPNRAPAGRVLLLNYIGGATNTGIVSKSASELVEAVDRDLRKMLINPSAVDPLVLGVRVWPQAIPQFNVGHLDLLEAAKSSLDRGGYDGLFLGGNYVAGVALGRCVEGAYESASQISDFLTKYAYK

SEQ ID NO: 152 - DNA, 13924 bp, synthetic construct
  source 1..13924: mol_type = other DNA; note = pBin TMV AtPPO1 S305L Y426V
agcttgcatgcctgcaggtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcttacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacctcgagtatttttacaacaattaccaacaacaacaaacaacaaacaacattacaattactatttacaattacacatatggagttatctcttctccgtccgacgactcaatcgcttcttccgtcgttttcgaagcccaatctccgattaaatgtttataagcctcttagactccgttgttcagtggccggtggaccaaccgtcggatcttcaaaaatcgaaggcggaggaggcaccaccatcacgacggattgtgtgattgtcggcggaggtattagtggtctttgcatcgctcaggcgcttgctactaagcatcctgatgctgctccgaatttaattgtgaccgaggctaaggatcgtgttggaggcaacattatcactcgtgaagagaatggttttctctgggaagaaggtcccaatagttttcaaccgtctgatcctatgctcactatggtggtagatagtggtttgaaggatgatttggtgttgggagatcctactgcgccaaggtttgtgttgtggaatgggaaattgaggccggttccatcgaagctaacagacttaccgttctttgatttgatgagtattggtgggaagattagagctggttttggtgcacttggcattcgaccgtcacctccaggtcgtgaagaatctgtggaggagtttgtacggcgtaacctcggtgatgaggtttttgagcgcctgattgaaccgttttgttcaggtgtttatgctggtgatccttcaaaactgagcatgaaagcagcgtttgggaaggtttggaaactagagcaaaatggtggaagcataataggtggtacttttaaggcaattcaggagaggaaaaacgctcccaaggcagaacgagacccgcgcctgccaaaaccacagggccaaacagttggttctttcaggaagggacttcgaatgttgccagaagcaatatctgcaagattaggtagcaaagttaagttgtcttggaagctccttggtatcactaagctggagagcggaggatacaacttaacatatgagactccagatggtttagtttccgtgcagagcaaaagtgttgtaatgacggtgccatctcatgttgcaagtggtctcttgcgccctctttctgaatctgctgcaaatgcactctcaaaactatattacccaccagttgcagcagtatctatctcgtacccgaaagaagcaatccgaacagaatgtttgatagatggtgaactaaagggttttgggcaattgcatccacgcacgcaaggagttgaaacattaggaactatctacagctcctcactctttccaaatcgcgcaccgcccggaagaattttgctgttgaacgttattggcgggtctacaaacaccggaattctgtccaagtctgaaggtgagttagtggaagcagttgacagagatttgaggaaaatgctaattaagcctaattcgaccgatccacttaaattaggagttagggtatggcctcaagccattcctcagtttctagttggtcactttgatatccttgacacggctaaatcatctctaacgtcttcgggctacgaagggctatttttgggtggcaattacgtcgctggtgtagccttaggccggtgtgtagaaggcgcatatgaaaccgcgattgaggtcaacaacttcatgtcacggtacgcttacaagtaaggtaccgagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattcctagagatcatgagcggagaattaagggagtcacgttatgacccccgccgatgacgcgggacaagccgttttacgtttggaactgacagaaccgcaacgttgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaatccaaataatctgcaccggatctggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgggatctctgcggaacaggcggtcgaaggtgccgatatcattacgacagcaacggccgacaagcacaacgccacgatcctgagcgacaatatgatcgggcccggcgtccacatcaacggcgtcggcggcgactgcccaggcaagaccgagatgcaccgcgatatcttgctgcgttcggatattttcgtggagttcccgccacagacccggatgatccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggcctcctgtcaatgctggcggcggctctggtggtggttctggtggcggctctgagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcggcctgaatggcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggaaccaccatcaaacaggattttcgcctgctggggcaaaccagcgtggaccgcttgctgcaactctctcagggccaggcggtgaagggcaatcagctgttgcccgtctcactggtgaaaagaaaaaccaccccagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaaatatcatctccctcgcagagatccgaattatcagccttcttattcatttctcgcttaaccgtgacaggctgtcgatcttgagaactatgccgacataataggaaatcgctggataaagccgctgaggaagctgagtggcgctatttctttagaagtgaacgttgacgatatcaactcccctatccattgctcaccgaatggtacaggtcggggacccgaagttccgactgtcggcctgatgcatccccggctgatcgaccccagatctggggctgagaaagcccagtaaggaaacaactgtaggttcgagtcgcgagatcccccggaaccaaaggaagtaggttaaacccgctccgatcaggccgagccacgccaggccgagaacattggttcctgtaggcatcgggattggcggatcaaacactaaagctactggaacgagcagaagtcctccggccgccagttgccaggcggtaaaggtgagcagaggcacgggaggttgccacttgcgggtcagcacggttccgaacgccatggaaaccgcccccgccaggcccgctgcgacgccgacaggatctagcgctgcgtttggtgtcaacaccaacagcgccacgcccgcagttccgcaaatagcccccaggaccgccatcaatcgtatcgggctacctagcagagcggcagagatgaacacgaccatcagcggctgcacagcgcctaccgtcgccgcgaccccgcccggcaggcggtagaccgaaataaacaacaagctccagaatagcgaaatattaagtgcgccgaggatgaagatgcgcatccaccagattcccgttggaatctgtcggacgatcatcacgagcaataaacccgccggcaacgcccgcagcagcataccggcgacccctcggcctcgctgttcgggctccacgaaaacgccggacagatgcgccttgtgagcgtccttggggccgtcctcctgtttgaagaccgacagcccaatgatctcgccgtcgatgtaggcgccgaatgccacggcatctcgcaaccgttcagcgaacgcctccatgggctttttctcctcgtgctcgtaaacggacccgaacatctctggagctttcttcagggccgacaatcggatctcgcggaaatcctgcacgtcggccgctccaagccgtcgaatctgagccttaatcacaattgtcaattttaatcctctgtttatcggcagttcgtagagcgcgccgtgcgtcccgagcgatactgagcgaagcaagtgcgtcgagcagtgcccgcttgttcctgaaatgccagtaaagcgctggctgctgaacccccagccggaactgaccccacaaggccctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcgagacgacgcgaacgctccacggcggccgatggcgcgggcagggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccccgactcacgccggggcaatgtgcccttattcctgatttgacccgcctggtgccttggtgtccagataatccaccttatcggcaatgaagtcggtcccgtagaccgtctggccgtccttctcgtacttggtattccgaatcttgccctgcacgaataccagcgaccccttgcccaaatacttgccgtgggcctcggcctgagagccaaaacacttgatgcggaagaagtcggtgcgctcctgcttgtcgccggcatcgttgcgccacatctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccacaattatgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctatctctgctctcactgccgtaaaacatggcaactgcagttcacttacaccgcttctcaacccggtacgcaccagaaaatcattgatatggccatgaatggcgttggatgccgggcaacagcccgcattatgggcgttggcctcaacacgattttacgtcacttaaaaaactcaggccgcagtcggtaacctcgcgcatacagccgggcagtgacgtcatcgtctgcgcggaaatggacgaacagtggggctatgtcggggctaaatcgcgccagcgctggctgttttacgcgtatgacagtctccggaagacggttgttgcgcacgtattcggtgaacgcactatggcgacgctggggcgtcttatgagcctgcttcaccctttgacgtggtgatatggatgacggatggctggccgctgtatgaatcccgcctgaagggaaagctgcacgtaatcagcaagcgatatacgcagcgaattgagcggcataacctgaatctgaggcagcacctggcacggctgggacggaagtcgctgtcgttctcaaaatcggtggagctgcatgacaaagtcatcgggcattatctgaacataaaacactatcaataagttggagtcattacccaattatgatagaatttacaagctataaggttattgtcctgggtttcaagcattagtccatgcaagtttttatgctttgcccattctatagatatattgataagcgcgctgcctatgccttgccccctgaaatccttacatacggcgatatcttctatataaaagatatattatcttatcagtattgtcaatatattcaaggcaatctgcctcctcatcctcttcatcctcttcgtcttggtagctttttaaatatggcgcttcatagagtaattctgtaaaggtccaattctcgttttcatacctcggtataatcttacctatcacctcaaatggttcgctgggtttatcgcacccccgaacacgagcacggcacccgcgaccactatgccaagaatgcccaaggtaaaaattgccggccccgccatgaagtccgtgaatgccccgacggccgaagtgaagggcaggccgccacccaggccgccgccctcactgcccggcacctggtcgctgaatgtcgatgccagcacctgcggcacgtcaatgcttccgggcgtcgcgctcgggctgatcgcccatcccgttactgccccgatcccggcaatggcaaggactgccagcgctgccatttttggggtgaggccgttcgcggccgaggggcgcagcccctggggggatgggaggcccgcgttagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccgcggtgtctcgcacacggcttcgacggcgtttctggcgcgtttgcagggccatagacggccgccagcccagcggcgagggcaaccagcccggtgagcgtcggaaaggcgctcggtcttgccttgctcgtcggtgatgtacttcaccagctccgcgaagtcgctcttcttgatggagcgcatggggacgtgcttggcaatcacgcgcaccccccggccgttttagcggctaaaaaagtcatggctctgccctcgggcggaccacgcccatcatgaccttgccaagctcgtcctgcttctcttcgatcttcgccagcagggcgaggatcgtggcatcaccgaaccgcgccgtgcgcgggtcgtcggtgagccagagtttcagcaggccgcccaggcggcccaggtcgccattgatgcgggccagctcgcggacgtgctcatagtccacgacgcccgtgattttgtagccctggccgacggccagcaggtaggccgacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgccagaaggccgccagagaggccgagcgcggccgtgaggcttggacgctagggcagggcatgaaaaagcccgtagcgggctgctacgggcgtctgacgcggtggaaagggggaggggatgttgtctacatggctctgctgtagtgagtgggttgcgctccggcagcggtcctgatcaatcgtcaccctttctcggtccttcaacgttcctgacaacgagcctccttttcgccaatccatcgacaatcaccgcgagtccctgctcgaacgctgcgtccggaccggcttcgtcgaaggcgtctatcgcggcccgcaacagcggcgagagcggagcctgttcaacggtgccgccgcgctcgccggcatcgctgtcgccggcctgctcctcaagcacggccccaacagtgaagtagctgattgtcatcagcgcattgacggcgtccccggccgaaaaacccgcctcgcagaggaagcgaagctgcgcgtcggccgtttccatctgcggtgcgcccggtcgcgtgccggcatggatgcgcgcgccatcgcggtaggcgagcagcgcctgcctgaagctgcgggcattcccgatcagaaatgagcgccagtcgtcgtcggctctcggcaccgaatgcgtatgattctccgccagcatggcttcggccagtgcgtcgagcagcgcccgcttgttcctgaagtgccagtaaagcgccggctgctgaacccccaaccgttccgccagtttgcgtgtcgtcagaccgtctacgccgacctcgttcaacaggtccagggcggcacggatcactgtattcggctgcaactttgtcatgcttgacactttatcactgataaacataatatgtccaccaacttatcagtgataaagaatccgcgcgttcaatcggaccagcggaggctggtccggaggccagacgtgaaacccaacatacccctgatcgtaattctgagcactgtcgcgctcgacgctgtcggcatcggcctgattatgccggtgctgccgggcctcctgcgcgatctggttcactcgaacgacgtcaccgcccactatggcattctgctggcgctgtatgcgttggtgcaatttgcctgcgcacctgtgctgggcgcgctgtcggatcgtttcgggcggcggccaatcttgctcgtctcgctggccggcgccagatctggggaaccctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatctctagga

SEQ ID NO: 153 - protein, 503 aa, Arabidopsis thaliana
  source 1..503: note = Arabidopsis thaliana PPO1
SVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 154 - protein, 500 aa, Setaria italica
  source 1..500: note = Setaria italica PPO1
AAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 155 - protein, 503 aa, synthetic construct
  source 1..503: note = Arabidopsis thaliana PPO1 G30A
SVAGGPTVGSSKIEGGGGTTITTDCVIVGAGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 156 - protein, 503 aa, synthetic construct
  source 1..503: note = Arabidopsis thaliana PPO1 V54I
SVAGGPTVGSSKIEGGGGTTITTDCVIVGGGISGLCIAQALATKHPDAAPNLIITEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 157 - protein, 503 aa, synthetic construct
  source 1..503: note = Arabidopsis thaliana PPO1 E77D
SVAGGPTVGSSKIEGGGGTTITTDCVIVGGGISGLCIAQALATKHPDAAPNLIVTEAKDRVGGNIITREENGFLWEDGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 158 - protein, 503 aa, synthetic construct
  source 1..503: note = Arabidopsis thaliana PPO1 E77F
SVAGGPTVGSSKIEGGGGTTITTDCVIVGGGISGLCIAQALATKHPDAAPNLIVTEAKDRVGGNIITREENGFLWEFGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 159 - protein, 503 aa, synthetic construct
  source 1..503: note = Arabidopsis thaliana PPO1 V93I
SVAGGPTVGSSKIEGGGGTTITTDCVIVGGGISGLCIAQALATKHPDAAPNLIVTEAKDRVGGNIITREENGFLWEEGPNSFQPSDPMLTMVIDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 160 - protein, 503 aa, synthetic construct
  source 1..503: note = Arabidopsis thaliana PPO1 V93L
SVAGGPTVGSSKIEGGGGTTITTDCVIVGGGISGLCIAQALATKHPDAAPNLIVTEAKDRVGGNIITREENGFLWEEGPNSFQPSDPMLTMVLDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 161 - protein, 503 aa, synthetic construct
  source 1..503: note = Arabidopsis thaliana PPO1 P120Y
SVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRYVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 162 - protein, 503 aa, synthetic construct
  source 1..503: note = Arabidopsis thaliana PPO1 K139H
SVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGHIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 163 - protein, 503 aa, synthetic construct
  source 1..503: note = Arabidopsis thaliana PPO1 A142L
SVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRLGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 164 - protein, 503 aa, synthetic construct
  source 1..503: note = Arabidopsis thaliana PPO1 V164W
SVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFWRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 165 - protein, 503 aa, synthetic construct
  source 1..503: note = Arabidopsis thaliana PPO1 V2172G
SVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEGFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 166 - protein, 503 aa, synthetic construct
  source 1..503: note = Arabidopsis thaliana PPO1 F172Y
SVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVYERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 167 - protein, 503 aa, synthetic construct
  source 1..503: note = Arabidopsis thaliana PPO1 E178Y
SVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIYPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 168 - protein, 503 aa, unidentified
  source 1..503: note = Arabidopsis thaliana PPO1 F180Y
SVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPYCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 169 - protein, 503 aa, synthetic construct
  source 1..503: note = Arabidopsis thaliana PPO1 V184K
SVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGKYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 170 - protein, 503 aa, synthetic construct
  source 1..503: note = Arabidopsis thaliana PPO1 Y185S
SVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVSAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 171 - protein, 503 aa, synthetic construct
  source 1..503: note = Arabidopsis thaliana PPO1 A186C
SVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYCGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 172 - protein, 503 aa, synthetic construct
  source 1..503: note = Arabidopsis thaliana PPO1 P237C
SVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKCQGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 173 - protein, 503 aa, synthetic construct
  source 1..503: note = Arabidopsis thaliana PPO1 Q238H
SVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPHGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 174 - protein, 503 aa, synthetic construct
  source 1..503: note = Arabidopsis thaliana PPO1 Q238I
SVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPIGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 175 - protein, 503 aa, synthetic construct
  source 1..503: note = Arabidopsis thaliana PPO1 Q238T
SVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPTGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 176 - protein, 503 aa, synthetic construct
  source 1..503: note = Arabidopsis thaliana PPO1 K247C
SVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRCGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 177 - protein, 503 aa, synthetic construct
  source 1..503: note = Arabidopsis thaliana PPO1 K247E
SVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFREGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 178 - protein, 503 aa, synthetic construct
  source 1..503: note = Arabidopsis thaliana PPO1 K247N
SVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRNGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 179 - protein, 503 aa, synthetic construct
  source 1..503: note = Arabidopsis thaliana PPO1 R250G
SVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLGMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 180 - protein, 503 aa, synthetic construct
  source 1..503: note = Arabidopsis thaliana PPO1 S271L
SVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLLGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 181 - protein, 503 aa, synthetic construct
  source 1..503: note = Arabidopsis thaliana PPO1 L326C
SVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKCYYPPVAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 182 - protein, 503 aa, synthetic construct
  source 1..503: note = Arabidopsis thaliana PPO1 Y327D
SVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLDYPPVAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 183 - protein, 503 aa, synthetic construct
  source 1..503: note = Arabidopsis thaliana PPO1 Y327F
SVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLFYPPVAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 184 - protein, 503 aa, synthetic construct
  source 1..503: note = Arabidopsis thaliana PPO1 Y327P
SVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLPYPPVAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 185 - protein, 503 aa, synthetic construct
  source 1..503: note = Arabidopsis thaliana PPO1 Y327Q
SVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLQYPPVAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 186 - protein, 503 aa, synthetic construct
  source 1..503: note = Arabidopsis thaliana PPO1 Y327R
SVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYRPVAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 187 - protein, 503 aa, synthetic construct
  source 1..503: note = Arabidopsis thaliana PPO1 Y327T
SVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLTYPPVAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 188 - protein, 503 aa, synthetic construct
  source 1..503: note = Arabidopsis thaliana PPO1 Y327F
SVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYFPPVAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 189 - protein, 503 aa, synthetic construct
  source 1..503: note = Arabidopsis thaliana PPO1 Y331L
SVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPLAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 190 - protein, 503 aa, synthetic construct
  source 1..503: note = Arabidopsis thaliana PPO1 Y331M
SVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPMAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 191 - protein, 503 aa, synthetic construct
  source 1..503: note = Arabidopsis thaliana PPO1 G370A
SVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLATIYSSSLFPNRAPPGRILLLNYIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 192 - protein, 503 aa, synthetic construct
  source 1..503: note = Arabidopsis thaliana PPO1 P379G
SVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFGNRAPPGRILLLNYIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 193 - protein, 503 aa, synthetic construct
  source 1..503: note = Arabidopsis thaliana PPO1 P348M
SVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPMGRILLLNYIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 194 - protein, 503 aa, synthetic construct
  source 1..503: note = Arabidopsis thaliana PPO1 L390V
SVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLVNYIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 195 - protein, 503 aa, synthetic construct
  source 1..503: note = Arabidopsis thaliana PPO1 Y392C
SVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNCIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 196 - protein, 503 aa, synthetic construct
  source 1..503: note = Arabidopsis thaliana PPO1 Y392L
SVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNLIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 197 - protein, 503 aa, synthetic construct
  source 1..503: note = Arabidopsis thaliana PPO1 Y392M
SVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNMIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 198 - protein, 503 aa, synthetic construct
  source 1..503: note = Arabidopsis thaliana PPO1 Y392T
SVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNTIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 199 - protein, 503 aa, synthetic construct
  source 1..503: note = Arabidopsis thaliana PPO1 Y392V
SVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNVIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 200 - protein, 503 aa, synthetic construct
  source 1..503: note = Arabidopsis thaliana PPO1 T413R
SVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNYIGGSRNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 201 - protein, 503 aa, synthetic construct
  source 1..503: note = Arabidopsis thaliana PPO1 V413C
SVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNYIGGSTNTGILSKSEGELVEACDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 202 - protein, 503 aa, synthetic construct
  source 1..503: note = Arabidopsis thaliana PPO1 V413I
SVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNYIGGSTNTGILSKSEGELVEAIDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 203 - protein, 503 aa, synthetic construct
  source 1..503: note = Arabidopsis thaliana PPO1 D414H
SVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNYIGGSTNTGILSKSEGELVEAVHRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 204 - protein, 503 aa, synthetic construct
  source 1..503: note = Arabidopsis thaliana PPO1 D414L
SVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNYIGGSTNTGILSKSEGELVEAVLRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 205 - protein, 503 aa, synthetic construct
  source 1..503: note = Arabidopsis thaliana PPO1 D414N
SVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNYIGGSTNTGILSKSEGELVEAVNRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 206 - protein, 503 aa, synthetic construct
  source 1..503: note = Arabidopsis thaliana PPO1 R415K
SVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNYIGGSTNTGILSKSEGELVEAVDKDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 207 - protein, 503 aa, synthetic construct
  source 1..503: note = Arabidopsis thaliana PPO1 T427Q
SVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNYIGGSTNTGILSKSEGELVEAVDRDLRKMLIKPNSQDPLKLGVRVWPQAIPQFLVGHFDILDTAKSSLTSSGYEGLFLGGNYVAGVALGRCVEGAYETAIEVNNFMSRYAYK

SEQ ID NO: 208 - protein, 503 aa, synthetic construct
  source 1..503: note = Arabidopsis thaliana PPO1 F444R
SVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQRLVGHFDILDTAKSSLTSSGYEGLFLGGNYVAGVALGRCVEGAYETAIEVNNFMSRYAYK

SEQ ID NO: 209 - protein, 503 aa, synthetic construct
  source 1..503: note = Arabidopsis thaliana PPO1 L445M
SVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFMVGHFDILDTAKSSLTSSGYEGLFLGGNYVAGVALGRCVEGAYETAIEVNNFMSRYAYK

SEQ ID NO: 210 - protein, 503 aa, synthetic construct
  source 1..503: note = Arabidopsis thaliana PPO1 L445N
SVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFNVGHFDILDTAKSSLTSSGYEGLFLGGNYVAGVALGRCVEGAYETAIEVNNFMSRYAYK

SEQ ID NO: 211 - protein, 503 aa, synthetic construct
  source 1..503: note = Arabidopsis thaliana PPO1 G467N
SVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFLVGHFDILDTAKSSLTSSGYEGLFLGGNYVANVALGRCVEGAYETAIEVNNFMSRYAYK

SEQ ID NO: 212 - protein, 503 aa, synthetic construct
  source 1..503: note = Arabidopsis thaliana PPO1 V477L
SVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFLVGHFDILDTAKSSLTSSGYEGLFLGGNYVAGLALGRCVEGAYETAIEVNNFMSRYAYK

SEQ ID NO: 213 - protein, 503 aa, synthetic construct
  source 1..503: note = Arabidopsis thaliana PPO1 V477N
SVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFLVGHFDILDTAKSSLTSSGYEGLFLGGNYVAGNALGRCVEGAYETAIEVNNFMSRYAYK

SEQ ID NO: 214 - protein, 503 aa, synthetic construct
  source 1..503: note = Arabidopsis thaliana PPO1 C482Q
SVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFLVGHFDILDTAKSSLTSSGYEGLFLGGNYVAGVALGRQVEGAYETAIEVNNFMSRYAYK

SEQ ID NO: 215 - protein, 500 aa, synthetic construct
  source 1..500: note = Setaria italica PPO1 G27A
AAVAGGAAEAPASAGARVSADCVVVGAGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 216 - protein, 500 aa, synthetic construct
  source 1..500: note = Setaria italica PPO1 V49I
AAVAGGAAEAPASAGARVSADCVVVGGGISGLCTAQALATKHGVGDVLITEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 217 - protein, 500 aa, synthetic construct
  source 1..500: note = Setaria italica PPO1 E74D
AAVAGGAAEAPASAGARVSADCVVVGGGISGLCTAQALATKHGVGDVLVTEARARPGGNITTVERPDEGYLWEDGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 218 - protein, 500 aa, synthetic construct
  source 1..500: note = Setaria italica PPO1 E74F
AAVAGGAAEAPASAGARVSADCVVVGGGISGLCTAQALATKHGVGDVLVTEARARPGGNITTVERPDEGYLWEFGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 219 - protein, 500 aa, synthetic construct
  source 1..500: note = Setaria italica PPO1 V90I
AAVAGGAAEAPASAGARVSADCVVVGGGISGLCTAQALATKHGVGDVLVTEARARPGGNITTVERPDEGYLWEEGPNSFQPSDPVLTMAIDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 220 - protein, 500 aa, synthetic construct
  source 1..500: note = Setaria italica PPO1 V90L
AAVAGGAAEAPASAGARVSADCVVVGGGISGLCTAQALATKHGVGDVLVTEARARPGGNITTVERPDEGYLWEEGPNSFQPSDPVLTMALDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 221 - protein, 500 aa, synthetic construct
  source 1..500: note = Setaria italica PPO1 P117Y
AAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRYVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 222 - protein, 500 aa, synthetic construct
  source 1..500: note = Setaria italica PPO1 K136H
AAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGHLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 223 - protein, 500 aa, synthetic construct
  source 1..500: note = Setaria italica PPO1 A139L
AAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRLGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 224 - protein, 500 aa, synthetic construct
  source 1..500: note = Setaria italica PPO1 V161W
AAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFWRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 225 - protein, 500 aa, synthetic construct
  source 1..500: note = Setaria italica PPO1 V169G
AAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEGFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 226 - protein, 500 aa, synthetic construct
  source 1..500: note = Setaria italica PPO1 F170Y
AAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVYERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 227 - protein, 500 aa, synthetic construct
  source 1..500: note = Setaria italica PPO1 E175Y
AAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIYPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 228 - protein, 500 aa, synthetic construct
  source 1..500: note = Setaria italica PPO1 F177Y
AAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPYCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 229 - protein, 500 aa, synthetic construct
  source 1..500: note = Setaria italica PPO1 V181K
AAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGKYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 230 - protein, 500 aa, synthetic construct
  source 1..500: note = Setaria italica PPO1 Y182S
AAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVSAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 231 - protein, 501 aa, synthetic construct
  source 1..501: note = Setaria italica PPO1 A183C
AAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYCAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 232 - protein, 500 aa, synthetic construct
  source 1..500: note = Setaria italica PPO1 P234C
AAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTCKGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 233 - protein, 500 aa, synthetic construct
  source 1..500: note = Setaria italica PPO1 K235H
AAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPHGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 234 - protein, 500 aa, synthetic construct
  source 1..500: note = Setaria italica PPO1 K235I
AAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPIGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 235 - protein, 500 aa, synthetic construct
  source 1..500: note = Setaria italica PPO1 K235T
AAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPTGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 236 - protein, 500 aa, synthetic construct
  source 1..500: note = Setaria italica PPO1 K244C
AAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRCGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 237 - protein, 500 aa, synthetic construct
  source 1..500: note = Setaria italica PPO1 K244E
AAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFREGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 238 - protein, 500 aa, synthetic construct
  source 1..500: note = Setaria italica PPO1 K244N
AAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRNGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 239 - protein, 500 aa, synthetic construct
  source 1..500: note = Setaria italica PPO1 A247G
AAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLGMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 240 - protein, 500 aa, synthetic construct
  source 1..500: note = Setaria italica PPO1 T268L
AAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLLSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 241 - protein, 500 aa, synthetic construct
  source 1..500: note = Setaria italica PPO1 F323C
AAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRCYYPPVAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 242 - protein, 500 aa, synthetic construct
  source 1..500: note = Setaria italica PPO1 Y324D
AAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFDYPPVAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 243 - protein, 500 aa, synthetic construct
  source 1..500: note = Setaria italica PPO1 Y324F
AAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFFYPPVAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 244 - protein, 500 aa, synthetic construct
  source 1..500: note = Setaria italica PPO1 Y324P
AAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFPYPPVAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 245 - protein, 500 aa, synthetic construct
  source 1..500: note = Setaria italica PPO1 Y324Q
AAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFQYPPVAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 246 - protein, 500 aa, synthetic construct
  source 1..500: note = Setaria italica PPO1 Y324R
AAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFRYPPVAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 247 - protein, 500 aa, synthetic construct
  source 1..500: note = Setaria italica PPO1 Y324T
AAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFTYPPVAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 248 - protein, 500 aa, synthetic construct
  source 1..500: note = Setaria italica PPO1 Y325F
AAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYFPPVAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 249 - protein, 500 aa, synthetic construct
  source 1..500: note = Setaria italica PPO1 V328L
AAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYYPPLAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 250 - protein, 500 aa, synthetic construct
  source 1..500: note = Setaria italica PPO1 V328M
AAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYYPPMAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 251 - protein, 500 aa, synthetic construct
  source 1..500: note = Setaria italica PPO1 G367A
AAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLATIYSSSLFPNRAPAGRVLLLNYIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 252 - protein, 500 aa, synthetic construct
  source 1..500: note = Setaria italica PPO1 P376G
AAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFGNRAPAGRVLLLNYIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 253 - protein, 500 aa, synthetic construct
  source 1..500: note = Setaria italica PPO1 A381M
AAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPMGRVLLLNYIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 254 - protein, 500 aa, synthetic construct
  source 1..500: note = Setaria italica PPO1 L387V
AAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLVNYIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 255 - protein, 500 aa, synthetic construct
  source 1..500: note = Setaria italica PPO1 Y389C
AAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNCIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 256 - protein, 500 aa, synthetic construct
  source 1..500: note = Setaria italica PPO1 Y389L
AAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNLIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 257 - protein, 500 aa, synthetic construct
  source 1..500: note = Setaria italica PPO1 Y389M
AAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNMIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 258 - protein, 500 aa, synthetic construct
  source 1..500: note = Setaria italica PPO1 Y389T
AAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNTIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 259 - protein, 500 aa, synthetic construct
  source 1..500: note = Setaria italica PPO1 Y389V
AAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNVIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 260 - protein, 500 aa, synthetic construct
  source 1..500: note = Setaria italica PPO1 T394R
AAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNYIGGARNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 261 - protein, 500 aa, synthetic construct
  source 1..500: note = Setaria italica PPO1 V410C
AAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNYIGGATNTGIVSKSASELVEACDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 262 - protein, 500 aa, synthetic construct
  source 1..500: note = Setaria italica PPO1 V410I
AAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNYIGGATNTGIVSKSASELVEAIDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 263 - protein, 500 aa, synthetic construct
  source 1..500: note = Setaria italica PPO1 D411H
AAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNYIGGATNTGIVSKSASELVEAVHRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 264 - protein, 500 aa, synthetic construct
  source 1..500: note = Setaria italica PPO1 D411L
AAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNYIGGATNTGIVSKSASELVEAVLRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 265 - protein, 500 aa, synthetic construct
  source 1..500: note = Setaria italica PPO1 D411N
AAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNYIGGATNTGIVSKSASELVEAVNRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 266 - protein, 500 aa, synthetic construct
  source 1..500: note = Setaria italica PPO1 R412K
AAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNYIGGATNTGIVSKSASELVEAVDKDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 267 - protein, 500 aa, synthetic construct
  source 1..500: note = Setaria italica PPO1 V424Q
AAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNYIGGATNTGIVSKSASELVEAVDRDLRKMLINPSAQDPLVLGVRVWPQAIPQFLVGHLDLLEAAKSSLDRGGYDGLFLGGNYVAGVALGRCVEGAYESASQISDFLTKYAYK

SEQ ID NO: 268 - protein, 500 aa, synthetic construct
  source 1..500: note = Setaria italica PPO1 F441R
AAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQRLVGHLDLLEAAKSSLDRGGYDGLFLGGNYVAGVALGRCVEGAYESASQISDFLTKYAYK

SEQ ID NO: 269 - protein, 500 aa, synthetic construct
  source 1..500: note = Setaria italica PPO1 L442M
AAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQFMVGHLDLLEAAKSSLDRGGYDGLFLGGNYVAGVALGRCVEGAYESASQISDFLTKYAYK

SEQ ID NO: 270 - protein, 500 aa, synthetic construct
  source 1..500: note = Setaria italica PPO1 L442N
AAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQFNVGHLDLLEAAKSSLDRGGYDGLFLGGNYVAGVALGRCVEGAYESASQISDFLTKYAYK

SEQ ID NO: 271 - protein, 500 aa, synthetic construct
  source 1..500: note = Setaria italica PPO1 G473N
AAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQFLVGHLDLLEAAKSSLDRGGYDGLFLGGNYVANVALGRCVEGAYESASQISDFLTKYAYK

SEQ ID NO: 272 - protein, 500 aa, synthetic construct
  source 1..500: note = Setaria italica PPO1 V474L
AAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQFLVGHLDLLEAAKSSLDRGGYDGLFLGGNYVAGLALGRCVEGAYESASQISDFLTKYAYK

SEQ ID NO: 273 - protein, 500 aa, synthetic construct
  source 1..500: note = Setaria italica PPO1 V474N
AAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQFLVGHLDLLEAAKSSLDRGGYDGLFLGGNYVAGNALGRCVEGAYESASQISDFLTKYAYK

SEQ ID NO: 274 - protein, 500 aa, synthetic construct
  source 1..500: note = Setaria italica PPO1 C479Q
AAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQFLVGHLDLLEAAKSSLDRGGYDGLFLGGNYVAGVALGRQVEGAYESASQISDFLTKYAYK

SEQ ID NO: 275 - protein, 503 aa, synthetic construct
  source 1..503: note = Arabidopsis thaliana PPO1 S271L Y392M
SVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLLGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNMIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 276 - protein, 503 aa, synthetic construct
  source 1..503: note = Arabidopsis thaliana PPO1 S271L Y392V
SVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLLGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNVIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 277 - protein, 503 aa, synthetic construct
  source 1..503: note = Arabidopsis thaliana PPO1 V331L Y392V L445N
SVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPLAAVSISYPKEAIRTECLIDGELKGFGQLHPRTQGVETLGTIYSSSLFPNRAPPGRILLLNVIGGSTNTGILSKSEGELVEAVDRDLRKMLIKPNSTDPLKLGVRVWPQAIPQFNVGHFDILDTAKSSLTSSGYEGLFLGGNYVAGVALGRCVEGAYETAIEVNNFMSRYAYK

SEQ ID NO: 278 - protein, 503 aa, synthetic construct
  source 1..503: note = Arabidopsis thaliana PPO1 S271L G370A Y392V T397R L445N
SVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLLGITKLESGGYNLTYETPDGLVSVQSKSVVMTVPSHVASGLLRPLSESAANALSKLYYPPVAAVSISYPKEAIRTECLIDGELKGFGQLHPRTQGVETLATIYSSSLFPNRAPPGRILLLNVIGGSRNTGILSKSEGELVEAVDRDLRKMLIKPNSTDPLKLGVRVWPQAIPQFNVGHFDILDTAKSSLTSSGYEGLFLGGNYVAGVALGRCVEGAYETAIEVNNFMSRYAYK

SEQ ID NO: 279 - protein, 503 aa, synthetic construct
  source 1..503: note = Arabidopsis thaliana PPO1 S271L V331M
SVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLLGITKLESGGYNLTYETPDGLVSVQSKSVVMTVPSHVASGLLRPLSESAANALSKLYYPPMAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 280 - protein, 503 aa, synthetic construct
  source 1..503: note = Arabidopsis thaliana PPO1 S271L Y328F G370A
SVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLLGITKLESGGYNLTYETPDGLVSVQSKSVVMTVPSHVASGLLRPLSESAANALSKLYFPPVAAVSISYPKEAIRTECLIDGELKGFGQLHPRTQGVETLATIYSSSLFPNRAPPGRILLLNYIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 281 - protein, 503 aa, synthetic construct
  source 1..503: note = Arabidopsis thaliana PPO1 S271L Y327T V331L T397R L445M
SVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLLGITKLESGGYNLTYETPDGLVSVQSKSVVMTVPSHVASGLLRPLSESAANALSKLTYPPLAAVSISYPKEAIRTECLIDGELKGFGQLHPRTQGVETLGTIYSSSLFPNRAPPGRILLLNYIGGSRNTGILSKSEGELVEAVDRDLRKMLIKPNSTDPLKLGVRVWPQAIPQFMVGHFDILDTAKSSLTSSGYEGLFLGGNYVAGVALGRCVEGAYETAIEVNNFMSRYAYK

SEQ ID NO: 282 - protein, 503 aa, synthetic construct
  source 1..503: note = Arabidopsis thaliana PPO1 S271L Y392V L445M
SVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLLGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNVIGGSTNTGILSKSEGELVEAVDRDLRKMLIKPNSTDPLKLGVRVWPQAIPQFMVGHFDILDTAKSSLTSSGYEGLFLGGNYVAGVALGRCVEGAYETAIEVNNFMSRYAYK

SEQ ID NO: 283 - protein, 503 aa, synthetic construct
  source 1..503: note = Arabidopsis thaliana PPO1 Y392M T427Q L445M
SVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNMIGGSTNTGILSKSEGELVEAVDRDLRKMLIKPNSQDPLKLGVRVWPQAIPQFMVGHFDILDTAKSSLTSSGYEGLFLGGNYVAGVALGRCVEGAYETAIEVNNFMSRYAYK

SEQ ID NO: 284 - protein, 503 aa, synthetic construct
  source 1..503: note = Arabidopsis thaliana PPO1 S271L Y327D V331M
SVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLLGITKLESGGYNLTYETPDGLVSVQSKSVVMTVPSHVASGLLRPLSESAANALSKLDYPPMAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 285 - protein, 503 aa, synthetic construct
  source 1..503: note = Arabidopsis thaliana PPO1 S271L Y327C Y328F Y392M L445M
SVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLLGITKLESGGYNLTYETPDGLVSVQSKSVVMTVPSHVASGLLRPLSESAANALSKLCFPPVAAVSISYPKEAIRTECLIDGELKGFGQLHPRTQGVETLGTIYSSSLFPNRAPPGRILLLNMIGGSTNTGILSKSEGELVEAVDRDLRKMLIKPNSTDPLKLGVRVWPQAIPQFMVGHFDILDTAKSSLTSSGYEGLFLGGNYVAGVALGRCVEGAYETAIEVNNFMSRYAYK

SEQ ID NO: 286 - protein, 503 aa, synthetic construct
  source 1..503: note = Arabidopsis thaliana PPO1 Y327D V331L
SVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLDYPPLAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 287 - protein, 503 aa, synthetic construct
  source 1..503: note = Arabidopsis thaliana PPO1 V331L G370A L445N
SVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPLAAVSISYPKEAIRTECLIDGELKGFGQLHPRTQGVETLATIYSSSLFPNRAPPGRILLLNYIGGSTNTGILSKSEGELVEAVDRDLRKMLIKPNSTDPLKLGVRVWPQAIPQFNVGHFDILDTAKSSLTSSGYEGLFLGGNYVAGVALGRCVEGAYETAIEVNNFMSRYAYK

SEQ ID NO: 288 - protein, 503 aa, synthetic construct
  source 1..503: note = Arabidopsis thaliana PPO1 S271L G370A L445N
SVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLLGITKLESGGYNLTYETPDGLVSVQSKSVVMTVPSHVASGLLRPLSESAANALSKLYYPPVAAVSISYPKEAIRTECLIDGELKGFGQLHPRTQGVETLATIYSSSLFPNRAPPGRILLLNYIGGSTNTGILSKSEGELVEAVDRDLRKMLIKPNSTDPLKLGVRVWPQAIPQFNVGHFDILDTAKSSLTSSGYEGLFLGGNYVAGVALGRCVEGAYETAIEVNNFMSRYAYK

SEQ ID NO: 289 - protein, 500 aa, synthetic construct
  source 1..500: note = Setaria italica PPO1 T268L Y389M
AAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLLSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNMIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 290 - protein, 500 aa, synthetic construct
  source 1..500: note = Setaria italica PPO1 T268L Y389V
AAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLLSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNVIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 291 - protein, 500 aa, synthetic construct
  source 1..500: note = Setaria italica PPO1 V328L Y389V L442N
AAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYYPPLAAVTISYPKEAIRKECLIDGELQGFGQLHPRSQGVETLGTIYSSSLFPNRAPAGRVLLLNVIGGATNTGIVSKSASELVEAVDRDLRKMLINPSAVDPLVLGVRVWPQAIPQFNVGHLDLLEAAKSSLDRGGYDGLFLGGNYVAGVALGRCVEGAYESASQISDFLTKYAYK

SEQ ID NO: 292 - protein, 500 aa, synthetic construct
  source 1..500: note = Setaria italica PPO1 T268L G367A Y389V T394R L442N
AAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLLSITKSDGMGYVLVYETPEGVVSVQAKSVIMTIPSYVASDILRPLSSDAADALSRFYYPPVAAVTISYPKEAIRKECLIDGELQGFGQLHPRSQGVETLATIYSSSLFPNRAPAGRVLLLNVIGGARNTGIVSKSASELVEAVDRDLRKMLINPSAVDPLVLGVRVWPQAIPQFNVGHLDLLEAAKSSLDRGGYDGLFLGGNYVAGVALGRCVEGAYESASQISDFLTKYAYK

SEQ ID NO: 293 - protein, 500 aa, synthetic construct
  source 1..500: note = Setaria italica PPO1 T268L V328M
AAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLLSITKSDGMGYVLVYETPEGVVSVQAKSVIMTIPSYVASDILRPLSSDAADALSRFYYPPMAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 294 - protein, 500 aa, synthetic construct
  source 1..500: note = Setaria italica PPO1 T268L Y325F G367A
AAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLLSITKSDGMGYVLVYETPEGVVSVQAKSVIMTIPSYVASDILRPLSSDAADALSRFYFPPVAAVTISYPKEAIRKECLIDGELQGFGQLHPRSQGVETLATIYSSSLFPNRAPAGRVLLLNYIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 295 - protein, 500 aa, synthetic construct
  source 1..500: note = Setaria italica PPO1 T268L Y324T V238L T394R L442M
AAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLLSITKSDGMGYVLVYETPEGVVSVQAKSVIMTIPSYVASDILRPLSSDAADALSRFTYPPLAAVTISYPKEAIRKECLIDGELQGFGQLHPRSQGVETLGTIYSSSLFPNRAPAGRVLLLNYIGGAANTGIVSKSASELVEAVDRDLRKMLINPSAVDPLVLGVRVWPQAIPQFMVGHLDLLEAAKSSLDRGGYDGLFLGGNYVAGVALGRCVEGAYESASQISDFLTKYAYK

SEQ ID NO: 296 - protein, 500 aa, synthetic construct
  source 1..500: note = Setaria italica PPO1 T268L Y389V L442M
AAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLLSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNVIGGATNTGIVSKSASELVEAVDRDLRKMLINPSAVDPLVLGVRVWPQAIPQFMVGHLDLLEAAKSSLDRGGYDGLFLGGNYVAGVALGRCVEGAYESASQISDFLTKYAYK

SEQ ID NO: 297 - protein, 500 aa, synthetic construct
  source 1..500: note = Setaria italica PPO1 Y389M V424Q L442M
AAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNMIGGATNTGIVSKSASELVEAVDRDLRKMLINPSAQDPLVLGVRVWPQAIPQFMVGHLDLLEAAKSSLDRGGYDGLFLGGNYVAGVALGRCVEGAYESASQISDFLTKYAYK

SEQ ID NO: 298 - protein, 500 aa, synthetic construct
  source 1..500: note = Setaria italica PPO1 T268L Y324D V328M
AAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLLSITKSDGMGYVLVYETPEGVVSVQAKSVIMTIPSYVASDILRPLSSDAADALSRFDYPPMAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 299 - protein, 500 aa, synthetic construct
  source 1..500: note = Setaria italica PPO1 T268L Y324C Y325F Y389M L442M
AAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLLSITKSDGMGYVLVYETPEGVVSVQAKSVIMTIPSYVASDILRPLSSDAADALSRFCFPPVAAVTISYPKEAIRKECLIDGELQGFGQLHPRSQGVETLGTIYSSSLFPNRAPAGRVLLLNMIGGATNTGIVSKSASELVEAVDRDLRKMLINPSAVDPLVLGVRVWPQAIPQFMVGHLDLLEAAKSSLDRGGYDGLFLGGNYVAGVALGRCVEGAYESASQISDFLTKYAYK

SEQ ID NO: 300 - protein, 500 aa, synthetic construct
  source 1..500: note = Setaria italica PPO1 Y324D V328L
AAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFDYPPLAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 301 - protein, 500 aa, synthetic construct
  source 1..500: note = Setaria italica PPO1 V328L G367A L442N
AAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYYPPLAAVTISYPKEAIRKECLIDGELQGFGQLHPRSQGVETLATIYSSSLFPNRAPAGRVLLLNYIGGATNTGIVSKSASELVEAVDRDLRKMLINPSAVDPLVLGVRVWPQAIPQFNVGHLDLLEAAKSSLDRGGYDGLFLGGNYVAGVALGRCVEGAYESASQISDFLTKYAYK

SEQ ID NO: 302 - protein, 500 aa, synthetic construct
  source 1..500: note = Setaria italica PPO1 T268L G367A L442N
AAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLLSITKSDGMGYVLVYETPEGVVSVQAKSVIMTIPSYVASDILRPLSSDAADALSRFYYPPVAAVTISYPKEAIRKECLIDGELQGFGQLHPRSQGVETLATIYSSSLFPNRAPAGRVLLLNYIGGATNTGIVSKSASELVEAVDRDLRKMLINPSAVDPLVLGVRVWPQAIPQFNVGHLDLLEAAKSSLDRGGYDGLFLGGNYVAGVALGRCVEGAYESASQISDFLTKYAYK

SEQ ID NO: 303 - protein, 34 aa, Arabidopsis thaliana
  source 1..34: note = Arabidopsis thaliana PPO1 WT chloroplastic transit peptide
MELSLLRPTTQSLLPSFSKPNLRLNVYKPLRLRC

SEQ ID NO: 304 - protein, 35 aa, Setaria italica
  source 1..35: note = Setaria italica PPO1 WT chloroplastic transit peptide
MVAAAMATAPSAGVPPLRGTRGPARFRIRGVSVRC

SEQ ID NO: 305 - protein, 537 aa, synthetic construct
MELSLLRPTTQSLLPSFSKPNLRLNVYKPLRLRCSVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPMPQGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 306 - protein, 537 aa, synthetic construct
MELSLLRPTTQSLLPSFSKPNLRLNVYKPLRLRCSVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLCYPPVAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 307 - protein, 537 aa, synthetic construct
MELSLLRPTTQSLLPSFSKPNLRLNVYKPLRLRCSVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYCPPVAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 308 - protein, 537 aa, synthetic construct
MELSLLRPTTQSLLPSFSKPNLRLNVYKPLRLRCSVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNYIGGSFNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 309 - protein, 537 aa, synthetic construct
MELSLLRPTTQSLLPSFSKPNLRLNVYKPLRLRCSVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPYKLGVRVWPQAIPQFLVGHFDILDTAKSSLTSSGYEGLFLGGNYVAGVALGRCVEGAYETAIEVNNFMSRYAYK

SEQ ID NO: 310 - protein, 537 aa, synthetic construct
MELSLLRPTTQSLLPSFSKPNLRLNVYKPLRLRCSVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFLVDHFDILDTAKSSLTSSGYEGLFLGGNYVAGVALGRCVEGAYETAIEVNNFMSRYAYK

SEQ ID NO: 311 - protein, 537 aa, synthetic construct
MELSLLRPTTQSLLPSFSKPNLRLNVYKPLRLRCSVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFLVKHFDILDTAKSSLTSSGYEGLFLGGNYVAGVALGRCVEGAYETAIEVNNFMSRYAYK

SEQ ID NO: 312 - protein, 537 aa, synthetic construct
MELSLLRPTTQSLLPSFSKPNLRLNVYKPLRLRCSVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFLVNHFDILDTAKSSLTSSGYEGLFLGGNYVAGVALGRCVEGAYETAIEVNNFMSRYAYK

SEQ ID NO: 313 - protein, 535 aa, synthetic construct
MVAAAMATAPSAGVPPLRGTRGPARFRIRGVSVRCAAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPMPKGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 314 - protein, 535 aa, synthetic construct
MVAAAMATAPSAGVPPLRGTRGPARFRIRGVSVRCAAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFCYPPVAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 315 - protein, 535 aa, synthetic construct
MVAAAMATAPSAGVPPLRGTRGPARFRIRGVSVRCAAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYCPPVAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 316 - protein, 535 aa, synthetic construct
MVAAAMATAPSAGVPPLRGTRGPARFRIRGVSVRCAAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNYIGGAFNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 317 - protein, 535 aa, synthetic construct
MVAAAMATAPSAGVPPLRGTRGPARFRIRGVSVRCAAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPYVLGVRVWPQAIPQFLVGHLDLLEAAKSSLDRGGYDGLFLGGNYVAGVALGRCVEGAYESASQISDFLTKYAYK

SEQ ID NO: 318 - protein, 535 aa, synthetic construct
MVAAAMATAPSAGVPPLRGTRGPARFRIRGVSVRCAAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQFLVDHLDLLEAAKSSLDRGGYDGLFLGGNYVAGVALGRCVEGAYESASQISDFLTKYAYK

SEQ ID NO: 319 - protein, 535 aa, synthetic construct
MVAAAMATAPSAGVPPLRGTRGPARFRIRGVSVRCAAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQFLVKHLDLLEAAKSSLDRGGYDGLFLGGNYVAGVALGRCVEGAYESASQISDFLTKYAYK

SEQ ID NO: 320 - protein, 535 aa, synthetic construct
MVAAAMATAPSAGVPPLRGTRGPARFRIRGVSVRCAAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQFLVNHLDLLEAAKSSLDRGGYDGLFLGGNYVAGVALGRCVEGAYESASQISDFLTKYAYK

SEQ ID NO: 321 - protein, 503 aa, synthetic construct
SVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPMPQGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 322 - protein, 503 aa, synthetic construct
SVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLCYPPVAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 323 - protein, 503 aa, synthetic construct
SVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYCPPVAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 324 - protein, 503 aa, synthetic construct
SVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNYIGGSFNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 325 - protein, 503 aa, synthetic construct
SVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPYKLGVRVWPQAIPQFLVGHFDILDTAKSSLTSSGYEGLFLGGNYVAGVALGRCVEGAYETAIEVNNFMSRYAYK

SEQ ID NO: 326 - protein, 503 aa, synthetic construct
SVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFLVDHFDILDTAKSSLTSSGYEGLFLGGNYVAGVALGRCVEGAYETAIEVNNFMSRYAYK

SEQ ID NO: 327 - protein, 503 aa, synthetic construct
SVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFLVKHFDILDTAKSSLTSSGYEGLFLGGNYVAGVALGRCVEGAYETAIEVNNFMSRYAYK

SEQ ID NO: 328 - protein, 503 aa, synthetic construct
SVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFLVNHFDILDTAKSSLTSSGYEGLFLGGNYVAGVALGRCVEGAYETAIEVNNFMSRYAYK

SEQ ID NO: 329 - protein, 500 aa, synthetic construct
AAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPMPKGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 330 - protein, 500 aa, synthetic construct
AAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFCYPPVAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 331 - protein, 500 aa, synthetic construct
AAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYCPPVAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 332 - protein, 500 aa, synthetic construct
AAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNYIGGAFNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 333 - protein, 500 aa, synthetic construct
AAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPYVLGVRVWPQAIPQFLVGHLDLLEAAKSSLDRGGYDGLFLGGNYVAGVALGRCVEGAYESASQISDFLTKYAYK

SEQ ID NO: 334 - protein, 500 aa, synthetic construct
AAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQFLVDHLDLLEAAKSSLDRGGYDGLFLGGNYVAGVALGRCVEGAYESASQISDFLTKYAYK

SEQ ID NO: 335 - protein, 500 aa, synthetic construct
AAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQFLVKHLDLLEAAKSSLDRGGYDGLFLGGNYVAGVALGRCVEGAYESASQISDFLTKYAYK

SEQ ID NO: 336 - protein, 500 aa, synthetic construct
AAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQFLVNHLDLLEAAKSSLDRGGYDGLFLGGNYVAGVALGRCVEGAYESASQISDFLTKYAYK

SEQ ID NO: 337 - protein, 14 aa, synthetic construct
  VARIANT 2: note = X is K, N or R
  VARIANT 3: note = X is L or V
  VARIANT 8: note = X is E, K or T
  VARIANT 11: note = X is G or S
  VARIANT 13: note = X is I, K, S or T
SXXKLSWXLLXIXK

SEQ ID NO: 338 - protein, 12 aa, synthetic construct
  VARIANT 2: note = X is A or S
  VARIANT 3: note = X is I, K, N, Q, R or S
  VARIANT 4: note = X is F or L
  VARIANT 5: note = X is D, F, P, Q, R or T
  VARIANT 10: note = X is A or G
  VARIANT 11: note = X is A or S
LXXXXYPPVXXV

SEQ ID NO: 339 - protein, 12 aa, synthetic construct
  VARIANT 2: note = X is A or S
  VARIANT 3: note = X is I, K, N, Q , R or S
  VARIANT 4: note = X is F or L
  VARIANT 5: note = X is D, F, H, or Y
  VARIANT 10: note = X is A or G
  VARIANT 11: note = X is A or S
LXXXXFPPVXXV

SEQ ID NO: 340 - protein, 12 aa, synthetic construct
  VARIANT 2: note = X is A or S
  VARIANT 3: note = X is I, K, N, Q , R or S
  VARIANT 4: note = X is F or L
  VARIANT 5: note = X is D, F, H, or Y
  VARIANT 9: note = X is L or M
  VARIANT 10: note = X is A or G
  VARIANT 11: note = X is A or S
LXXXXYPPXXXV

SEQ ID NO: 341 - protein, 15 aa, synthetic construct
  VARIANT 2: note = X is I, L or V
  VARIANT 7: note = X is S or T
GXETLAXIYSSSLFP

SEQ ID NO: 342 - protein, 22 aa, synthetic construct
  VARIANT 4: note = X is G or N
  VARIANT 8: note = X is A, D, K, N, P or S
  VARIANT 10: note = X is Q or R
  VARIANT 11: note = X is I, T or V
  VARIANT 12: note = X is M or L
  VARIANT 13: note = X is I or L
  VARIANT 15: note = X is N or S
  VARIANT 16: note = X is C, L, M, T or V
  VARIANT 20: note = X is A or S
  VARIANT 21: note = X is K, L or T
LFPXRAPXGXXXXLXXIGGXXN

SEQ ID NO: 343 - protein, 22 aa, synthetic construct
  VARIANT 4: note = X is G or N
  VARIANT 8: note = X is A, D, K, N, P or S
  VARIANT 10: note = X is Q or R
  VARIANT 11: note = X is I, T or V
  VARIANT 12: note = X is M or L
  VARIANT 13: note = X is I or L
  VARIANT 15: note = X is N or S
  VARIANT 16: note = X is F or Y
  VARIANT 20: note = X is A or S
LFPXRAPXGXXXXLXXIGGXRN

SEQ ID NO: 344 - protein, 24 aa, synthetic construct
  VARIANT 1: note = X is G, P or S
  VARIANT 2: note = X is D, K, N, R, S, or T
  VARIANT 3: note = X is A or S
  VARIANT 5: note = X is D or E
  VARIANT 7: note = X is F, L, R or S
  VARIANT 8: note = X is A, T or V
  VARIANT 9: note = X is L, T or V
  VARIANT 10: note = X is G or S
  VARIANT 12: note = X is K or R
  VARIANT 15: note = X is P or R
  VARIANT 16: note = X is K, Q or R
  VARIANT 23: note = X is I or V
XXXQXPXXXXVXVWXXAIPQFLXG

SEQ ID NO: 345 - protein, 24 aa, synthetic construct
  VARIANT 1: note = X is G, P or S
  VARIANT 2: note = X is D, K, N, R, S, or T
  VARIANT 3: note = X is A or S
  VARIANT 4: note = X is A, E, G, K, Q or V
  VARIANT 5: note = X is D or E
  VARIANT 7: note = X is F, L, R or S
  VARIANT 8: note = X is A, T or V
  VARIANT 9: note = X is L, T or V
  VARIANT 10: note = X is G or S
  VARIANT 12: note = X is K or R
  VARIANT 15: note = X is P or R
  VARIANT 16: note = X is K, Q or R
  VARIANT 22: note = X is M or N
  VARIANT 23: note = X is I or V
XXXXXPXXXXVXVWXXAIPQFXXG

SEQ ID NO: 346 - protein, 9 aa, synthetic construct
  VARIANT 5: note = X is E, K or T
  VARIANT 8: note = X is G or S
KLSWXLLXI

SEQ ID NO: 347 - protein, 8 aa, synthetic construct
  VARIANT 1: note = X is D, F, P, Q, R or T
  VARIANT 6: note = X is A or G
  VARIANT 7: note = X is A or S
XYPPVXXV

SEQ ID NO: 348 - protein, 9 aa, synthetic construct
  VARIANT 1: note = X is F or L
  VARIANT 2: note = X is D, F, H or Y
  VARIANT 7: note = X is A or G
  VARIANT 8: note = X is A or S
XXFPPVXXV

SEQ ID NO: 349 - protein, 7 aa, synthetic construct
  VARIANT 4: note = X is L or M
  VARIANT 5: note = X is A or G
  VARIANT 6: note = X is A or S
YPPXXXV

SEQ ID NO: 350 - protein, 13 aa, synthetic construct
  VARIANT 5: note = X is S or A
ETLAXIYSSSLFP

SEQ ID NO: 351 - protein, 9 aa, synthetic construct
  VARIANT 2: note = X is N or S
  VARIANT 3: note = X is C, L, M, T, V
  VARIANT 7: note = X is A or S
  VARIANT 8: note = X is K, L or T
LXXIGGXXN

SEQ ID NO: 352 - protein, 9 aa, synthetic construct
  VARIANT 2: note = X is N or S
  VARIANT 3: note = X isF or Y
  VARIANT 7: note = X is A or S
LXXIGGXRN

SEQ ID NO: 353 - protein, 20 aa, synthetic construct
  VARIANT 1: note = X is A or S
  VARIANT 3: note = X is D or E
  VARIANT 5: note = X is F, L, R or S
  VARIANT 6: note = X is A, T or V
  VARIANT 7: note = X is L, T or V
  VARIANT 8: note = X is G or S
  VARIANT 10: note = X is K or R
  VARIANT 13: note = X is P or R
  VARIANT 14: note = X is K, Q or R
XQXPXXXXVXVWXXAIPQFL

SEQ ID NO: 354 - protein, 24 aa, synthetic construct
  VARIANT 1: note = X is G, P or S
  VARIANT 2: note = X is D, K, N, R, S, or T
  VARIANT 3: note = X is A or S
  VARIANT 4: note = X is A, E, G, K, Q, V or T
  VARIANT 5: note = X is D or E
  VARIANT 7: note = X is F, L, R or S
  VARIANT 8: note = X is A, T, V or K
  VARIANT 9: note = X is L, T or V
  VARIANT 10: note = X is G or S
  VARIANT 12: note = X is K or R
  VARIANT 15: note = X is P or R
  VARIANT 16: note = X is K, Q or R
  VARIANT 22: note = X is M or N
  VARIANT 23: note = X is I or V
XXXXXPXXXXVXVWXXAIPQFXXG

SEQ ID NO: 355 - protein, 8 aa, synthetic construct
  VARIANT 6: note = X is M or N
  VARIANT 7: note = X is I or V
AIPQFXXG

SEQ ID NO: 356 - DNA, 1614 bp, synthetic construct
  source 1..1614: mol_type = other DNA
atggagttatctcttctccgtccgacgactcaatcgcttcttccgtcgttttcgaagcccaatctccgattaaatgtttataagcctcttagactccgttgttcagtggccggtggaccaaccgtcggatcttcaaaaatcgaaggcggaggaggcaccaccatcacgacggattgtgtgattgtcggcggaggtattagtggtctttgcatcgctcaggcgcttgctactaagcatcctgatgctgctccgaatttaattgtgaccgaggctaaggatcgtgttggaggcaacattatcactcgtgaagagaatggttttctctgggaagaaggtcccaatagttttcaaccgtctgatcctatgctcactatggtggtagatagtggtttgaaggatgatttggtgttgggagatcctactgcgccaaggtttgtgttgtggaatgggaaattgaggccggttccatcgaagctaacagacttaccgttctttgatttgatgagtattggtgggaagattagagctggttttggtgcacttggcattcgaccgtcacctccaggtcgtgaagaatctgtggaggagtttgtacggcgtaacctcggtgatgaggtttttgagcgcctgattgaaccgttttgttcaggtgtttatgctggtgatccttcaaaactgagcatgaaagcagcgtttgggaaggtttggaaactagagcaaaatggtggaagcataataggtggtacttttaaggcaattcaggagaggaaaaacgctcccaaggcagaacgagacccgcgcctgccaaaaccacagggccaaacagttggttctttcaggaagggacttcgaatgttgccagaagcaatatctgcaagattaggtagcaaagttaagttgtcttggaagctccttggtatcactaagctggagagcggaggatacaacttaacatatgagactccagatggtttagtttccgtgcagagcaaaagtgttgtaatgacggtgccatctcatgttgcaagtggtctcttgcgccctctttctgaatctgctgcaaatgcactctcaaaactatatttcccaccagttgcagcagtatctatctcgtacccgaaagaagcaatccgaacagaatgtttgatagatggtgaactaaagggttttgggcaattgcatccacgcacgcaaggagttgaaacattagctactatctacagctcctcactctttccaaatcgcgcaccgcccggaagaattttgctgttgaactacattggcgggtctacaaacaccggaattctgtccaagtctgaaggtgagttagtggaagcagttgacagagatttgaggaaaatgctaattaagcctaattcgaccgatccacttaaattaggagttagggtatggcctcaagccattcctcagtttctagttggtcactttgatatccttgacacggctaaatcatctctaacgtcttcgggctacgaagggctatttttgggtggcaattacgtcgctggtgtagccttaggccggtgtgtagaaggcgcatatgaaaccgcgattgaggtcaacaacttcatgtcacggtacgcttacaagtaa

SEQ ID NO: 357 - DNA, 1614 bp, synthetic construct
  source 1..1614: mol_type = other DNA
atggagttatctcttctccgtccgacgactcaatcgcttcttccgtcgttttcgaagcccaatctccgattaaatgtttataagcctcttagactccgttgttcagtggccggtggaccaaccgtcggatcttcaaaaatcgaaggcggaggaggcaccaccatcacgacggattgtgtgattgtcggcggaggtattagtggtctttgcatcgctcaggcgcttgctactaagcatcctgatgctgctccgaatttaattgtgaccgaggctaaggatcgtgttggaggcaacattatcactcgtgaagagaatggttttctctgggaagaaggtcccaatagttttcaaccgtctgatcctatgctcactatggtggtagatagtggtttgaaggatgatttggtgttgggagatcctactgcgccaaggtttgtgttgtggaatgggaaattgaggccggttccatcgaagctaacagacttaccgttctttgatttgatgagtattggtgggaagattagagctggttttggtgcacttggcattcgaccgtcacctccaggtcgtgaagaatctgtggaggagtttgtacggcgtaacctcggtgatgaggtttttgagcgcctgattgaaccgttttgttcaggtgtttatgctggtgatccttcaaaactgagcatgaaagcagcgtttgggaaggtttggaaactagagcaaaatggtggaagcataataggtggtacttttaaggcaattcaggagaggaaaaacgctcccaaggcagaacgagacccgcgcctgccaaaaccacagggccaaacagttggttctttcaggaagggacttcgaatgttgccagaagcaatatctgcaagattaggtagcaaagttaagttgtcttggaagctctcaggtatcactaagctggagagcggaggatacaacttaacatatgagactccagatggtttagtttccgtgcagagcaaaagtgttgtaatgacggtgccatctcatgttgcaagtggtctcttgcgccctctttctgaatctgctgcaaatgcactctcaaaactatattacccaccacttgcagcagtatctatctcgtacccgaaagaagcaatccgaacagaatgtttgatagatggtgaactaaagggttttgggcaattgcatccacgcacgcaaggagttgaaacattagctactatctacagctcctcactctttccaaatcgcgcaccgcccggaagaattttgctgttgaactacattggcgggtctacaaacaccggaattctgtccaagtctgaaggtgagttagtggaagcagttgacagagatttgaggaaaatgctaattaagcctaattcgaccgatccacttaaattaggagttagggtatggcctcaagccattcctcagtttaatgttggtcactttgatatccttgacacggctaaatcatctctaacgtcttcgggctacgaagggctatttttgggtggcaattacgtcgctggtgtagccttaggccggtgtgtagaaggcgcatatgaaaccgcgattgaggtcaacaacttcatgtcacggtacgcttacaagtaa

SEQ ID NO: 358 - DNA, 1614 bp, synthetic construct
  source 1..1614: mol_type = other DNA
atggagttatctcttctccgtccgacgactcaatcgcttcttccgtcgttttcgaagcccaatctccgattaaatgtttataagcctcttagactccgttgttcagtggccggtggaccaaccgtcggatcttcaaaaatcgaaggcggaggaggcaccaccatcacgacggattgtgtgattgtcggcggaggtattagtggtctttgcatcgctcaggcgcttgctactaagcatcctgatgctgctccgaatttaattgtgaccgaggctaaggatcgtgttggaggcaacattatcactcgtgaagagaatggttttctctgggaagaaggtcccaatagttttcaaccgtctgatcctatgctcactatggtggtagatagtggtttgaaggatgatttggtgttgggagatcctactgcgccaaggtttgtgttgtggaatgggaaattgaggccggttccatcgaagctaacagacttaccgttctttgatttgatgagtattggtgggaagattagagctggttttggtgcacttggcattcgaccgtcacctccaggtcgtgaagaatctgtggaggagtttgtacggcgtaacctcggtgatgaggtttttgagcgcctgattgaaccgttttgttcaggtgtttatgctggtgatccttcaaaactgagcatgaaagcagcgtttgggaaggtttggaaactagagcaaaatggtggaagcataataggtggtacttttaaggcaattcaggagaggaaaaacgctcccaaggcagaacgagacccgcgcctgccaaaaccacagggccaaacagttggttctttcaggaagggacttcgaatgttgccagaagcaatatctgcaagattaggtagcaaagttaagttgtcttggaagctctcaggtatcactaagctggagagcggaggatacaacttaacatatgagactccagatggtttagtttccgtgcagagcaaaagtgttgtaatgacggtgccatctcatgttgcaagtggtctcttgcgccctctttctgaatctgctgcaaatgcactctcaaaactatattacccaccagttgcagcagtatctatctcgtacccgaaagaagcaatccgaacagaatgtttgatagatggtgaactaaagggttttgggcaattgcatccacgcacgcaaggagttgaaacattaggaactatctacagctcctcactctttccaaatcgcgcaccgcccggaagaattttgctgttgaacatgattggcgggtctacaaacaccggaattctgtccaagtctgaaggtgagttagtggaagcagttgacagagatttgaggaaaatgctaattaagcctaattcgcaagatccacttaaattaggagttagggtatggcctcaagccattcctcagtttatggttggtcactttgatatccttgacacggctaaatcatctctaacgtcttcgggctacgaagggctatttttgggtggcaattacgtcgctggtgtagccttaggccggtgtgtagaaggcgcatatgaaaccgcgattgaggtcaacaacttcatgtcacggtacgcttacaagtaa

SEQ ID NO: 359 - DNA, 1614 bp, synthetic construct
  source 1..1614: mol_type = other DNA
atggagttatctcttctccgtccgacgactcaatcgcttcttccgtcgttttcgaagcccaatctccgattaaatgtttataagcctcttagactccgttgttcagtggccggtggaccaaccgtcggatcttcaaaaatcgaaggcggaggaggcaccaccatcacgacggattgtgtgattgtcggcggaggtattagtggtctttgcatcgctcaggcgcttgctactaagcatcctgatgctgctccgaatttaattgtgaccgaggctaaggatcgtgttggaggcaacattatcactcgtgaagagaatggttttctctgggaagaaggtcccaatagttttcaaccgtctgatcctatgctcactatggtggtagatagtggtttgaaggatgatttggtgttgggagatcctactgcgccaaggtttgtgttgtggaatgggaaattgaggccggttccatcgaagctaacagacttaccgttctttgatttgatgagtattggtgggaagattagagctggttttggtgcacttggcattcgaccgtcacctccaggtcgtgaagaatctgtggaggagtttgtacggcgtaacctcggtgatgaggtttttgagcgcctgattgaaccgttttgttcaggtgtttatgctggtgatccttcaaaactgagcatgaaagcagcgtttgggaaggtttggaaactagagcaaaatggtggaagcataataggtggtacttttaaggcaattcaggagaggaaaaacgctcccaaggcagaacgagacccgcgcctgccaaaaccacagggccaaacagttggttctttcaggaagggacttcgaatgttgccagaagcaatatctgcaagattaggtagcaaagttaagttgtcttggaagctctcaggtatcactaagctggagagcggaggatacaacttaacatatgagactccagatggtttagtttccgtgcagagcaaaagtgttgtaatgacggtgccatctcatgttgcaagtggtctcttgcgccctctttctgaatctgctgcaaatgcactctcaaaactagattacccaccacttgcagcagtatctatctcgtacccgaaagaagcaatccgaacagaatgtttgatagatggtgaactaaagggttttgggcaattgcatccacgcacgcaaggagttgaaacattaggaactatctacagctcctcactctttccaaatcgcgcaccgcccggaagaattttgctgttgaactacattggcgggtctacaaacaccggaattctgtccaagtctgaaggtgagttagtggaagcagttgacagagatttgaggaaaatgctaattaagcctaattcgaccgatccacttaaattaggagttagggtatggcctcaagccattcctcagtttctagttggtcactttgatatccttgacacggctaaatcatctctaacgtcttcgggctacgaagggctatttttgggtggcaattacgtcgctggtgtagccttaggccggtgtgtagaaggcgcatatgaaaccgcgattgaggtcaacaacttcatgtcacggtacgcttacaagtaa

SEQ ID NO: 360 - DNA, 1614 bp, synthetic construct
  source 1..1614: mol_type = other DNA
atggagttatctcttctccgtccgacgactcaatcgcttcttccgtcgttttcgaagcccaatctccgattaaatgtttataagcctcttagactccgttgttcagtggccggtggaccaaccgtcggatcttcaaaaatcgaaggcggaggaggcaccaccatcacgacggattgtgtgattgtcggcggaggtattagtggtctttgcatcgctcaggcgcttgctactaagcatcctgatgctgctccgaatttaattgtgaccgaggctaaggatcgtgttggaggcaacattatcactcgtgaagagaatggttttctctgggaagaaggtcccaatagttttcaaccgtctgatcctatgctcactatggtggtagatagtggtttgaaggatgatttggtgttgggagatcctactgcgccaaggtttgtgttgtggaatgggaaattgaggccggttccatcgaagctaacagacttaccgttctttgatttgatgagtattggtgggaagattagagctggttttggtgcacttggcattcgaccgtcacctccaggtcgtgaagaatctgtggaggagtttgtacggcgtaacctcggtgatgaggtttttgagcgcctgattgaaccgttttgttcaggtgtttatgctggtgatccttcaaaactgagcatgaaagcagcgtttgggaaggtttggaaactagagcaaaatggtggaagcataataggtggtacttttaaggcaattcaggagaggaaaaacgctcccaaggcagaacgagacccgcgcctgccaaaaccacagggccaaacagttggttctttcaggaagggacttcgaatgttgccagaagcaatatctgcaagattaggtagcaaagttaagttgtcttggaagctccttggtatcactaagctggagagcggaggatacaacttaacatatgagactccagatggtttagtttccgtgcagagcaaaagtgttgtaatgacggtgccatctcatgttgcaagtggtctcttgcgccctctttctgaatctgctgcaaatgcactctcaaaactatattacccaccagttgcagcagtatctatctcgtacccgaaagaagcaatccgaacagaatgtttgatagatggtgaactaaagggttttgggcaattgcatccacgcacgcaaggagttgaaacattagctactatctacagctcctcactctttccaaatcgcgcaccgcccggaagaattttgctgttgaacgttattggcgggtctagaaacaccggaattctgtccaagtctgaaggtgagttagtggaagcagttgacagagatttgaggaaaatgctaattaagcctaattcgaccgatccacttaaattaggagttagggtatggcctcaagccattcctcagtttaatgttggtcactttgatatccttgacacggctaaatcatctctaacgtcttcgggctacgaagggctatttttgggtggcaattacgtcgctggtgtagccttaggccggtgtgtagaaggcgcatatgaaaccgcgattgaggtcaacaacttcatgtcacggtacgcttacaagtaa

SEQ ID NO: 361 - DNA, 1614 bp, synthetic construct
  source 1..1614: mol_type = other DNA
atggagttatctcttctccgtccgacgactcaatcgcttcttccgtcgttttcgaagcccaatctccgattaaatgtttataagcctcttagactccgttgttcagtggccggtggaccaaccgtcggatcttcaaaaatcgaaggcggaggaggcaccaccatcacgacggattgtgtgattgtcggcggaggtattagtggtctttgcatcgctcaggcgcttgctactaagcatcctgatgctgctccgaatttaattgtgaccgaggctaaggatcgtgttggaggcaacattatcactcgtgaagagaatggttttctctgggaagaaggtcccaatagttttcaaccgtctgatcctatgctcactatggtggtagatagtggtttgaaggatgatttggtgttgggagatcctactgcgccaaggtttgtgttgtggaatgggaaattgaggccggttccatcgaagctaacagacttaccgttctttgatttgatgagtattggtgggaagattagagctggttttggtgcacttggcattcgaccgtcacctccaggtcgtgaagaatctgtggaggagtttgtacggcgtaacctcggtgatgaggtttttgagcgcctgattgaaccgttttgttcaggtgtttatgctggtgatccttcaaaactgagcatgaaagcagcgtttgggaaggtttggaaactagagcaaaatggtggaagcataataggtggtacttttaaggcaattcaggagaggaaaaacgctcccaaggcagaacgagacccgcgcctgccaaaaccacagggccaaacagttggttctttcaggaagggacttcgaatgttgccagaagcaatatctgcaagattaggtagcaaagttaagttgtcttggaagctccttggtatcactaagctggagagcggaggatacaacttaacatatgagactccagatggtttagtttccgtgcagagcaaaagtgttgtaatgacggtgccatctcatgttgcaagtggtctcttgcgccctctttctgaatctgctgcaaatgcactctcaaaactatattacccaccaatggcagcagtatctatctcgtacccgaaagaagcaatccgaacagaatgtttgatagatggtgaactaaagggttttgggcaattgcatccacgcacgcaaggagttgaaacattaggaactatctacagctcctcactctttccaaatcgcgcaccgcccggaagaattttgctgttgaactacattggcgggtctacaaacaccggaattctgtccaagtctgaaggtgagttagtggaagcagttgacagagatttgaggaaaatgctaattaagcctaattcgaccgatccacttaaattaggagttagggtatggcctcaagccattcctcagtttctagttggtcactttgatatccttgacacggctaaatcatctctaacgtcttcgggctacgaagggctatttttgggtggcaattacgtcgctggtgtagccttaggccggtgtgtagaaggcgcatatgaaaccgcgattgaggtcaacaacttcatgtcacggtacgcttacaagtaa

SEQ ID NO: 362 - DNA, 1614 bp, synthetic construct
  source 1..1614: mol_type = other DNA
atggagttatctcttctccgtccgacgactcaatcgcttcttccgtcgttttcgaagcccaatctccgattaaatgtttataagcctcttagactccgttgttcagtggccggtggaccaaccgtcggatcttcaaaaatcgaaggcggaggaggcaccaccatcacgacggattgtgtgattgtcggcggaggtattagtggtctttgcatcgctcaggcgcttgctactaagcatcctgatgctgctccgaatttaattgtgaccgaggctaaggatcgtgttggaggcaacattatcactcgtgaagagaatggttttctctgggaagaaggtcccaatagttttcaaccgtctgatcctatgctcactatggtggtagatagtggtttgaaggatgatttggtgttgggagatcctactgcgccaaggtttgtgttgtggaatgggaaattgaggccggttccatcgaagctaacagacttaccgttctttgatttgatgagtattggtgggaagattagagctggttttggtgcacttggcattcgaccgtcacctccaggtcgtgaagaatctgtggaggagtttgtacggcgtaacctcggtgatgaggtttttgagcgcctgattgaaccgttttgttcaggtgtttatgctggtgatccttcaaaactgagcatgaaagcagcgtttgggaaggtttggaaactagagcaaaatggtggaagcataataggtggtacttttaaggcaattcaggagaggaaaaacgctcccaaggcagaacgagacccgcgcctgccaaaaccacagggccaaacagttggttctttcaggaagggacttcgaatgttgccagaagcaatatctgcaagattaggtagcaaagttaagttgtcttggaagctccttggtatcactaagctggagagcggaggatacaacttaacatatgagactccagatggtttagtttccgtgcagagcaaaagtgttgtaatgacggtgccatctcatgttgcaagtggtctcttgcgccctctttctgaatctgctgcaaatgcactctcaaaactatattacccaccagttgcagcagtatctatctcgtacccgaaagaagcaatccgaacagaatgtttgatagatggtgaactaaagggttttgggcaattgcatccacgcacgcaaggagttgaaacattagctactatctacagctcctcactctttccaaatcgcgcaccgcccggaagaattttgctgttgaactacattggcgggtctacaaacaccggaattctgtccaagtctgaaggtgagttagtggaagcagttgacagagatttgaggaaaatgctaattaagcctaattcgaccgatccacttaaattaggagttagggtatggcctcaagccattcctcagtttaatgttggtcactttgatatccttgacacggctaaatcatctctaacgtcttcgggctacgaagggctatttttgggtggcaattacgtcgctggtgtagccttaggccggtgtgtagaaggcgcatatgaaaccgcgattgaggtcaacaacttcatgtcacggtacgcttacaagtaa

SEQ ID NO: 363 - DNA, 1614 bp, synthetic construct
  source 1..1614: mol_type = other DNA
atggagttatctcttctccgtccgacgactcaatcgcttcttccgtcgttttcgaagcccaatctccgattaaatgtttataagcctcttagactccgttgttcagtggccggtggaccaaccgtcggatcttcaaaaatcgaaggcggaggaggcaccaccatcacgacggattgtgtgattgtcggcggaggtattagtggtctttgcatcgctcaggcgcttgctactaagcatcctgatgctgctccgaatttaattgtgaccgaggctaaggatcgtgttggaggcaacattatcactcgtgaagagaatggttttctctgggaagaaggtcccaatagttttcaaccgtctgatcctatgctcactatggtggtagatagtggtttgaaggatgatttggtgttgggagatcctactgcgccaaggtttgtgttgtggaatgggaaattgaggccggttccatcgaagctaacagacttaccgttctttgatttgatgagtattggtgggaagattagagctggttttggtgcacttggcattcgaccgtcacctccaggtcgtgaagaatctgtggaggagtttgtacggcgtaacctcggtgatgaggtttttgagcgcctgattgaaccgttttgttcaggtgtttatgctggtgatccttcaaaactgagcatgaaagcagcgtttgggaaggtttggaaactagagcaaaatggtggaagcataataggtggtacttttaaggcaattcaggagaggaaaaacgctcccaaggcagaacgagacccgcgcctgccaaaaccacagggccaaacagttggttctttcaggaagggacttcgaatgttgccagaagcaatatctgcaagattaggtagcaaagttaagttgtcttggaagctccttggtatcactaagctggagagcggaggatacaacttaacatatgagactccagatggtttagtttccgtgcagagcaaaagtgttgtaatgacggtgccatctcatgttgcaagtggtctcttgcgccctctttctgaatctgctgcaaatgcactctcaaaactaacttacccaccacttgcagcagtatctatctcgtacccgaaagaagcaatccgaacagaatgtttgatagatggtgaactaaagggttttgggcaattgcatccacgcacgcaaggagttgaaacattaggaactatctacagctcctcactctttccaaatcgcgcaccgcccggaagaattttgctgttgaactacattggcgggtctagaaacaccggaattctgtccaagtctgaaggtgagttagtggaagcagttgacagagatttgaggaaaatgctaattaagcctaattcgaccgatccacttaaattaggagttagggtatggcctcaagccattcctcagtttatggttggtcactttgatatccttgacacggctaaatcatctctaacgtcttcgggctacgaagggctatttttgggtggcaattacgtcgctggtgtagccttaggccggtgtgtagaaggcgcatatgaaaccgcgattgaggtcaacaacttcatgtcacggtacgcttacaagtaa

SEQ ID NO: 364 - DNA, 1614 bp, synthetic construct
  source 1..1614: mol_type = other DNA
atggagttatctcttctccgtccgacgactcaatcgcttcttccgtcgttttcgaagcccaatctccgattaaatgtttataagcctcttagactccgttgttcagtggccggtggaccaaccgtcggatcttcaaaaatcgaaggcggaggaggcaccaccatcacgacggattgtgtgattgtcggcggaggtattagtggtctttgcatcgctcaggcgcttgctactaagcatcctgatgctgctccgaatttaattgtgaccgaggctaaggatcgtgttggaggcaacattatcactcgtgaagagaatggttttctctgggaagaaggtcccaatagttttcaaccgtctgatcctatgctcactatggtggtagatagtggtttgaaggatgatttggtgttgggagatcctactgcgccaaggtttgtgttgtggaatgggaaattgaggccggttccatcgaagctaacagacttaccgttctttgatttgatgagtattggtgggaagattagagctggttttggtgcacttggcattcgaccgtcacctccaggtcgtgaagaatctgtggaggagtttgtacggcgtaacctcggtgatgaggtttttgagcgcctgattgaaccgttttgttcaggtgtttatgctggtgatccttcaaaactgagcatgaaagcagcgtttgggaaggtttggaaactagagcaaaatggtggaagcataataggtggtacttttaaggcaattcaggagaggaaaaacgctcccaaggcagaacgagacccgcgcctgccaaaaccacagggccaaacagttggttctttcaggaagggacttcgaatgttgccagaagcaatatctgcaagattaggtagcaaagttaagttgtcttggaagctctcaggtatcactaagctggagagcggaggatacaacttaacatatgagactccagatggtttagtttccgtgcagagcaaaagtgttgtaatgacggtgccatctcatgttgcaagtggtctcttgcgccctctttctgaatctgctgcaaatgcactctcaaaactatattacccaccacttgcagcagtatctatctcgtacccgaaagaagcaatccgaacagaatgtttgatagatggtgaactaaagggttttgggcaattgcatccacgcacgcaaggagttgaaacattaggaactatctacagctcctcactctttccaaatcgcgcaccgcccggaagaattttgctgttgaacgttattggcgggtctacaaacaccggaattctgtccaagtctgaaggtgagttagtggaagcagttgacagagatttgaggaaaatgctaattaagcctaattcgaccgatccacttaaattaggagttagggtatggcctcaagccattcctcagtttaatgttggtcactttgatatccttgacacggctaaatcatctctaacgtcttcgggctacgaagggctatttttgggtggcaattacgtcgctggtgtagccttaggccggtgtgtagaaggcgcatatgaaaccgcgattgaggtcaacaacttcatgtcacggtacgcttacaagtaa

SEQ ID NO: 365 - DNA, 1614 bp, synthetic construct
  source 1..1614: mol_type = other DNA
atggagttatctcttctccgtccgacgactcaatcgcttcttccgtcgttttcgaagcccaatctccgattaaatgtttataagcctcttagactccgttgttcagtggccggtggaccaaccgtcggatcttcaaaaatcgaaggcggaggaggcaccaccatcacgacggattgtgtgattgtcggcggaggtattagtggtctttgcatcgctcaggcgcttgctactaagcatcctgatgctgctccgaatttaattgtgaccgaggctaaggatcgtgttggaggcaacattatcactcgtgaagagaatggttttctctgggaagaaggtcccaatagttttcaaccgtctgatcctatgctcactatggtggtagatagtggtttgaaggatgatttggtgttgggagatcctactgcgccaaggtttgtgttgtggaatgggaaattgaggccggttccatcgaagctaacagacttaccgttctttgatttgatgagtattggtgggaagattagagctggttttggtgcacttggcattcgaccgtcacctccaggtcgtgaagaatctgtggaggagtttgtacggcgtaacctcggtgatgaggtttttgagcgcctgattgaaccgttttgttcaggtgtttatgctggtgatccttcaaaactgagcatgaaagcagcgtttgggaaggtttggaaactagagcaaaatggtggaagcataataggtggtacttttaaggcaattcaggagaggaaaaacgctcccaaggcagaacgagacccgcgcctgccaaaaccacagggccaaacagttggttctttcaggaagggacttcgaatgttgccagaagcaatatctgcaagattaggtagcaaagttaagttgtcttggaagctccttggtatcactaagctggagagcggaggatacaacttaacatatgagactccagatggtttagtttccgtgcagagcaaaagtgttgtaatgacggtgccatctcatgttgcaagtggtctcttgcgccctctttctgaatctgctgcaaatgcactctcaaaactatattacccaccagttgcagcagtatctatctcgtacccgaaagaagcaatccgaacagaatgtttgatagatggtgaactaaagggttttgggcaattgcatccacgcacgcaaggagttgaaacattaggaactatctacagctcctcactctttccaaatcgcgcaccgcccggaagaattttgctgttgaacgttattggcgggtctacaaacaccggaattctgtccaagtctgaaggtgagttagtggaagcagttgacagagatttgaggaaaatgctaattaagcctaattcgaccgatccacttaaattaggagttagggtatggcctcaagccattcctcagtttatggttggtcactttgatatccttgacacggctaaatcatctctaacgtcttcgggctacgaagggctatttttgggtggcaattacgtcgctggtgtagccttaggccggtgtgtagaaggcgcatatgaaaccgcgattgaggtcaacaacttcatgtcacggtacgcttacaagtaa

SEQ ID NO: 366 - DNA, 1614 bp, synthetic construct
  source 1..1614: mol_type = other DNA
atggagttatctcttctccgtccgacgactcaatcgcttcttccgtcgttttcgaagcccaatctccgattaaatgtttataagcctcttagactccgttgttcagtggccggtggaccaaccgtcggatcttcaaaaatcgaaggcggaggaggcaccaccatcacgacggattgtgtgattgtcggcggaggtattagtggtctttgcatcgctcaggcgcttgctactaagcatcctgatgctgctccgaatttaattgtgaccgaggctaaggatcgtgttggaggcaacattatcactcgtgaagagaatggttttctctgggaagaaggtcccaatagttttcaaccgtctgatcctatgctcactatggtggtagatagtggtttgaaggatgatttggtgttgggagatcctactgcgccaaggtttgtgttgtggaatgggaaattgaggccggttccatcgaagctaacagacttaccgttctttgatttgatgagtattggtgggaagattagagctggttttggtgcacttggcattcgaccgtcacctccaggtcgtgaagaatctgtggaggagtttgtacggcgtaacctcggtgatgaggtttttgagcgcctgattgaaccgttttgttcaggtgtttatgctggtgatccttcaaaactgagcatgaaagcagcgtttgggaaggtttggaaactagagcaaaatggtggaagcataataggtggtacttttaaggcaattcaggagaggaaaaacgctcccaaggcagaacgagacccgcgcctgccaaaaccacagggccaaacagttggttctttcaggaagggacttcgaatgttgccagaagcaatatctgcaagattaggtagcaaagttaagttgtcttggaagctccttggtatcactaagctggagagcggaggatacaacttaacatatgagactccagatggtttagtttccgtgcagagcaaaagtgttgtaatgacggtgccatctcatgttgcaagtggtctcttgcgccctctttctgaatctgctgcaaatgcactctcaaaactatattacccaccagttgcagcagtatctatctcgtacccgaaagaagcaatccgaacagaatgtttgatagatggtgaactaaagggttttgggcaattgcatccacgcacgcaaggagttgaaacattaggaactatctacagctcctcactctttccaaatcgcgcaccgcccggaagaattttgctgttgaacgttattggcgggtctacaaacaccggaattctgtccaagtctgaaggtgagttagtggaagcagttgacagagatttgaggaaaatgctaattaagcctaattcgaccgatccacttaaattaggagttagggtatggcctcaagccattcctcagtttctagttggtcactttgatatccttgacacggctaaatcatctctaacgtcttcgggctacgaagggctatttttgggtggcaattacgtcgctggtgtagccttaggccggtgtgtagaaggcgcatatgaaaccgcgattgaggtcaacaacttcatgtcacggtacgcttacaagtaa

SEQ ID NO: 367 - DNA, 1614 bp, synthetic construct
  source 1..1614: mol_type = other DNA
atggagttatctcttctccgtccgacgactcaatcgcttcttccgtcgttttcgaagcccaatctccgattaaatgtttataagcctcttagactccgttgttcagtggccggtggaccaaccgtcggatcttcaaaaatcgaaggcggaggaggcaccaccatcacgacggattgtgtgattgtcggcggaggtattagtggtctttgcatcgctcaggcgcttgctactaagcatcctgatgctgctccgaatttaattgtgaccgaggctaaggatcgtgttggaggcaacattatcactcgtgaagagaatggttttctctgggaagaaggtcccaatagttttcaaccgtctgatcctatgctcactatggtggtagatagtggtttgaaggatgatttggtgttgggagatcctactgcgccaaggtttgtgttgtggaatgggaaattgaggccggttccatcgaagctaacagacttaccgttctttgatttgatgagtattggtgggaagattagagctggttttggtgcacttggcattcgaccgtcacctccaggtcgtgaagaatctgtggaggagtttgtacggcgtaacctcggtgatgaggtttttgagcgcctgattgaaccgttttgttcaggtgtttatgctggtgatccttcaaaactgagcatgaaagcagcgtttgggaaggtttggaaactagagcaaaatggtggaagcataataggtggtacttttaaggcaattcaggagaggaaaaacgctcccaaggcagaacgagacccgcgcctgccaaaaccacagggccaaacagttggttctttcaggaagggacttcgaatgttgccagaagcaatatctgcaagattaggtagcaaagttaagttgtcttggaagctccttggtatcactaagctggagagcggaggatacaacttaacatatgagactccagatggtttagtttccgtgcagagcaaaagtgttgtaatgacggtgccatctcatgttgcaagtggtctcttgcgccctctttctgaatctgctgcaaatgcactctcaaaactatgtttcccaccagttgcagcagtatctatctcgtacccgaaagaagcaatccgaacagaatgtttgatagatggtgaactaaagggttttgggcaattgcatccacgcacgcaaggagttgaaacattaggaactatctacagctcctcactctttccaaatcgcgcaccgcccggaagaattttgctgttgaacatgattggcgggtctacaaacaccggaattctgtccaagtctgaaggtgagttagtggaagcagttgacagagatttgaggaaaatgctaattaagcctaattcgaccgatccacttaaattaggagttagggtatggcctcaagccattcctcagtttatggttggtcactttgatatccttgacacggctaaatcatctctaacgtcttcgggctacgaagggctatttttgggtggcaattacgtcgctggtgtagccttaggccggtgtgtagaaggcgcatatgaaaccgcgattgaggtcaacaacttcatgtcacggtacgcttacaagtaa

SEQ ID NO: 368 - DNA, 1614 bp, synthetic construct
  source 1..1614: mol_type = other DNA
atggagttatctcttctccgtccgacgactcaatcgcttcttccgtcgttttcgaagcccaatctccgattaaatgtttataagcctcttagactccgttgttcagtggccggtggaccaaccgtcggatcttcaaaaatcgaaggcggaggaggcaccaccatcacgacggattgtgtgattgtcggcggaggtattagtggtctttgcatcgctcaggcgcttgctactaagcatcctgatgctgctccgaatttaattgtgaccgaggctaaggatcgtgttggaggcaacattatcactcgtgaagagaatggttttctctgggaagaaggtcccaatagttttcaaccgtctgatcctatgctcactatggtggtagatagtggtttgaaggatgatttggtgttgggagatcctactgcgccaaggtttgtgttgtggaatgggaaattgaggccggttccatcgaagctaacagacttaccgttctttgatttgatgagtattggtgggaagattagagctggttttggtgcacttggcattcgaccgtcacctccaggtcgtgaagaatctgtggaggagtttgtacggcgtaacctcggtgatgaggtttttgagcgcctgattgaaccgttttgttcaggtgtttatgctggtgatccttcaaaactgagcatgaaagcagcgtttgggaaggtttggaaactagagcaaaatggtggaagcataataggtggtacttttaaggcaattcaggagaggaaaaacgctcccaaggcagaacgagacccgcgcctgccaaaaccacagggccaaacagttggttctttcaggaagggacttcgaatgttgccagaagcaatatctgcaagattaggtagcaaagttaagttgtcttggaagctccttggtatcactaagctggagagcggaggatacaacttaacatatgagactccagatggtttagtttccgtgcagagcaaaagtgttgtaatgacggtgccatctcatgttgcaagtggtctcttgcgccctctttctgaatctgctgcaaatgcactctcaaaactagattacccaccaatggcagcagtatctatctcgtacccgaaagaagcaatccgaacagaatgtttgatagatggtgaactaaagggttttgggcaattgcatccacgcacgcaaggagttgaaacattaggaactatctacagctcctcactctttccaaatcgcgcaccgcccggaagaattttgctgttgaactacattggcgggtctacaaacaccggaattctgtccaagtctgaaggtgagttagtggaagcagttgacagagatttgaggaaaatgctaattaagcctaattcgaccgatccacttaaattaggagttagggtatggcctcaagccattcctcagtttctagttggtcactttgatatccttgacacggctaaatcatctctaacgtcttcgggctacgaagggctatttttgggtggcaattacgtcgctggtgtagccttaggccggtgtgtagaaggcgcatatgaaaccgcgattgaggtcaacaacttcatgtcacggtacgcttacaagtaa

SEQ ID NO: 369 - DNA, 1614 bp, Arabidopsis thaliana
  source 1..1614: mol_type = genomic DNA
atggagttatctcttctccgtccgacgactcaatcgcttcttccgtcgttttcgaagcccaatctccgattaaatgtttataagcctcttagactccgttgttcagtggccggtggaccaaccgtcggatcttcaaaaatcgaaggcggaggaggcaccaccatcacgacggattgtgtgattgtcggcggaggtattagtggtctttgcatcgctcaggcgcttgctactaagcatcctgatgctgctccgaatttaattgtgaccgaggctaaggatcgtgttggaggcaacattatcactcgtgaagagaatggttttctctgggaagaaggtcccaatagttttcaaccgtctgatcctatgctcactatggtggtagatagtggtttgaaggatgatttggtgttgggagatcctactgcgccaaggtttgtgttgtggaatgggaaattgaggccggttccatcgaagctaacagacttaccgttctttgatttgatgagtattggtgggaagattagagctggttttggtgcacttggcattcgaccgtcacctccaggtcgtgaagaatctgtggaggagtttgtacggcgtaacctcggtgatgaggtttttgagcgcctgattgaaccgttttgttcaggtgtttatgctggtgatccttcaaaactgagcatgaaagcagcgtttgggaaggtttggaaactagagcaaaatggtggaagcataataggtggtacttttaaggcaattcaggagaggaaaaacgctcccaaggcagaacgagacccgcgcctgccaaaaccacagggccaaacagttggttctttcaggaagggacttcgaatgttgccagaagcaatatctgcaagattaggtagcaaagttaagttgtcttggaagctctcaggtatcactaagctggagagcggaggatacaacttaacatatgagactccagatggtttagtttccgtgcagagcaaaagtgttgtaatgacggtgccatctcatgttgcaagtggtctcttgcgccctctttctgaatctgctgcaaatgcactctcaaaactatattacccaccagttgcagcagtatctatctcgtacccgaaagaagcaatccgaacagaatgtttgatagatggtgaactaaagggttttgggcaattgcatccacgcacgcaaggagttgaaacattaggaactatctacagctcctcactctttccaaatcgcgcaccgcccggaagaattttgctgttgaactacattggcgggtctacaaacaccggaattctgtccaagtctgaaggtgagttagtggaagcagttgacagagatttgaggaaaatgctaattaagcctaattcgaccgatccacttaaattaggagttagggtatggcctcaagccattcctcagtttctagttggtcactttgatatccttgacacggctaaatcatctctaacgtcttcgggctacgaagggctatttttgggtggcaattacgtcgctggtgtagccttaggccggtgtgtagaaggcgcatatgaaaccgcgattgaggtcaacaacttcatgtcacggtacgcttacaagtaa

SEQ ID NO: 370 - protein, 522 aa, synthetic construct
MGSSHHHHHHSSGENLYFQSVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 371 - protein, 522 aa, synthetic construct
MGSSHHHHHHSSGENLYFQSVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFMVGHFDILDTAKSSLTSSGYEGLFLGGNYVAGVALGRCVEGAYETAIEVNNFMSRYAYK

SEQ ID NO: 372 - protein, 522 aa, synthetic construct
MGSSHHHHHHSSGENLYFQSVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFNVGHFDILDTAKSSLTSSGYEGLFLGGNYVAGVALGRCVEGAYETAIEVNNFMSRYAYK

SEQ ID NO: 373 - protein, 522 aa, synthetic construct
MGSSHHHHHHSSGENLYFQSVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLLGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNVIGGSTNTGILSKSEGELVEAVDRDLRKMLIKPNSTDPLKLGVRVWPQAIPQFMVGHFDILDTAKSSLTSSGYEGLFLGGNYVAGVALGRCVEGAYETAIEVNNFMSRYAYK

SEQ ID NO: 374 - protein, 522 aa, synthetic construct
MGSSHHHHHHSSGENLYFQSVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLLGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNVIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 375 - protein, 6 aa, synthetic construct
HHHHHH

SEQ ID NO: 376 - protein, 7 aa, synthetic construct
ENLYFQS

SEQ ID NO: 377 - protein, 13 aa, synthetic construct
HHHHHHENLYFQS

SEQ ID NO: 378 - protein, 20 aa, synthetic construct
MGSSHHHHHHSSGENLYFQS

(19) United States
(12) Patent Application Publication
(10) Pub. No.: US 2026/0297542 A1
(43) Pub. Date: Oct. 1, 2026

(54) HERBICIDE RESISTANT PLANTS

(71) Applicant: SYNGENTA CROP PROTECTION AG, BASEL (CH)

(72) Inventors: Richard Paul DALE, Bracknell, Berkshire (GB); Anthea Karin BATCHELOR, Bracknell, Berkshire (GB); Rachael Elizabeth BLAIN, Bracknell, Berkshire (GB); Marta Andreia Horta SIMOES, Bracknell, Berkshire (GB); Sophie OLIVER, Bracknell, Berkshire (GB)

(73) Assignee: SYNGENTA CROP PROTECTION AG, Basel (CH)

(21) Appl. No.: 19/476,388

(22) PCT Filed: Apr. 18, 2024
(86) PCT No.: PCT/EP2024/060566

§ 371 Date: Oct. 17, 2025

(30) Foreign Application Priority Data
Apr. 19, 2023 (EP) 23168834.2

(51) Int. Cl. C12N 9/02; C12N 15/82

(52) U.S. Cl. CPC C12N 9/001; C12N 15/8274; C12Y 103/03004

(57) ABSTRACT

The invention relates to modified Protoporphyrinogen Oxidase (PPO) enzymes or functional fragment thereof that have increased resistance to PPO inhibiting compounds, plants or parts thereof including the modified PPO enzymes, uses thereof and methods of controlling undesired vegetation using the plants or parts thereof.

### FIELD OF THE INVENTION

The present invention relates to modified protoporphyrinogen oxidase (PPO) enzymes having one or more mutations at specific amino acid positions which increase the resistance of the enzyme to PPO-inhibiting compounds such as herbicides. The invention further relates to plants or parts thereof comprising the modified PPO enzymes, methods of making the plants, and methods of controlling undesired vegetation in the vicinity of said plants.

### BACKGROUND

The present invention relates to the production of modified PPO enzymes, and plants containing said enzymes, that are resistant to herbicides.

Protoporphyrinogen oxidase (PPO) enzymes (EC 1.3.3.4), are well conserved eukaryotic enzymes which convert protoporphyrinogen IX to protoporphyrin IX. PPO is a flavoprotein located in the inner mitochondrial membrane, which catalyses the removal of six hydrogens (four from methylene bridges and two from ring nitrogens) to form protoporphyrin-IX. This involves a three-step, six-electron flavin adenine dinucleotide-dependent oxidation that consumes molecular oxygen. It is essential to mitochondrial heme functions.

Herbicides of particular interest in the agricultural field include those that inhibit PPO enzymes, referred to as PPO inhibiting herbicides. Given that heme function is essential to the survival of plants, it has been identified as a herbicidal target. Recently, effective herbicidal compounds which inhibit PPO enzymes have been developed. Upon contact with plants, the herbicides cause cell death and eventually death of the plant. Industrially, herbicides are used in agriculture to remove unwanted vegetation such as weeds from cultivated crops. However, non-specific herbicides which target essential pathways, such as those that target heme synthesis, will affect both crops and the unwanted vegetation, making them difficult to use without destroying the valuable crop. Therefore in order to effectively use these herbicides, it would be desirable to protect the crop plants so that they have resistance to the herbicidal compounds. Plants that have resistance to the herbicides can then be contacted with the herbicide and will not be affected, whilst non-resistant unwanted vegetation is affected and controlled.

Agricultural crop production often utilizes transgenic traits created using the methods of biotechnology. A heterologous gene, also known as a transgene, can be introduced into a plant to produce a transgenic trait. Expression of the transgene in the plant confers a trait, such as herbicide tolerance, to the plant. Herbicide tolerant traits allow a plant to grow, whilst non-tolerant weed species are eradicated by contact with the herbicide. Examples of transgenic herbicide tolerance traits include glyphosate tolerance, glufosinate tolerance, and dicamba tolerance. With the increase of weed species resistant to the commonly used herbicides, new herbicides and accompanying new herbicide tolerance traits are needed in the field. Given that various herbicides which target PPO have been developed, there is thus a need to develop traits conferring tolerance to PPO-inhibiting herbicides, which will be particularly useful in crops.

The present invention aims to solve one or more of the above-mentioned problems in the art.

### STATEMENTS OF INVENTION

According to a first aspect of the present invention, there is provided a modified Protoporphyrinogen Oxidase (PPO) enzyme or functional fragment thereof, wherein the modified Protoporphyrinogen Oxidase or functional fragment thereof comprises at least one mutation at one or more amino acid residues selected from residues 362, 365 and/or 479 of SEQ ID NO:1, or residues corresponding thereto.

In a second aspect of the present invention, there is provided a modified Protoporphyrinogen Oxidase (PPO) enzyme or functional fragment thereof, wherein the modified Protoporphyrinogen Oxidase or functional fragment thereof comprises a mutation at amino acid residues 305 and 426 of SEQ ID NO:1, or residues corresponding thereto, wherein the mutation at amino acid residue 305, or a residue corresponding thereto, is a substitution to leucine (305L) and wherein the mutation at amino acid residue 426, or a residue corresponding thereto, is a substitution to valine (426V). In certain embodiments, the modified PPO enzyme comprises substitutions S305L and Y426V with reference to SEQ ID NO: 1 or residue corresponding thereto. In certain embodiments, the modified PPO enzyme comprises substitutions T305L and Y426V with reference to SEQ ID NO: 2 or residue corresponding thereto. In certain embodiments, the modified PPO enzyme comprises or consists of an amino acid sequence according to SEQ ID NO: 125. In certain embodiments, the modified PPO enzyme comprises or consists of an amino acid sequence according to SEQ ID NO: 139.

According to a third aspect of the present invention, there is provided a nucleic acid comprising a polynucleotide encoding a modified PPO enzyme or functional fragment thereof according to the first or second aspect.

According to a fourth aspect of the present invention, there is provided a recombinant vector comprising a nucleic acid according to the third aspect.

According to a fifth aspect of the present invention, there is provided a plant or part thereof comprising a modified PPO enzyme or functional fragment thereof according to the first or second aspect, a nucleic acid according to the third aspect, or a recombinant vector according to the fourth aspect.

According to a sixth aspect of the present invention, there is provided a method of producing a plant or part thereof according to the fifth aspect, the method comprising: modifying the plant or part thereof to comprise a PPO enzyme according to the first or second aspect.

According to a seventh aspect of the present invention, there is provided a method of controlling undesired vegetation in the vicinity of a plant or part thereof according to the fifth aspect, or at a locus for growth of a plant according to the fifth aspect, the method comprising applying an effective amount of at least one compound which inhibits a PPO enzyme to the undesired vegetation, the plant or part thereof, and/or the locus, and/or optionally planting a seed at the locus wherein the seed is capable of producing a plant according to the fifth aspect.

The invention will now be further described under the following headed sections, features under any given section may apply to any embodiment or aspect in any combination.

### DETAILED DESCRIPTION

### Modified PPO Enzymes

The invention primarily relates to modified Protoporphyrinogen Oxidase (PPO) enzymes that have increased resistance to compounds such as herbicides, in comparison to wild type or control PPO enzymes.

As used herein, reference to a “PPO enzyme” or “modified PPO enzyme” and the amino acid sequences thereof also refers to and is intended to encompass isolated polynucleotides encoding such an enzyme. The terms “modified polynucleotide”, “mutated polynucleotide”, “mutated polypeptide”, or “modified polypeptide” is a polynucleotide or polypeptide that has been altered through human intervention. Such a “mutated polynucleotide”, “modified polynucleotide”, “mutated polypeptide”, or “modified polypeptide” has a sequence that differs from the sequence of the corresponding non-mutated or non-modified polynucleotide or polypeptide (for example a wild type or naturally occurring polynucleotide or polypeptide) by at least one nucleotide or amino acid. In certain examples, the mutated polynucleotide or modified polynucleotide comprises an alteration that results from a guide polynucleotide/Cas endonuclease system as disclosed herein.

Protoporphyrinogen Oxidase (PPO) refers to a key enzyme in the biosynthesis of protoporphyrin IX. PPO catalyzes the last common step in chlorophyll and heme biosynthesis, which is the oxidation of protoporphyrinogen IX to protoporphyrin IX. (Matringe et al. 1989. Biochem. 1. 260: 231). Inhibition of protoporphyrinogen oxidase is a mechanism of action for several commercial herbicides, including the nitrophenyl ethers acifluorfen and fomesafen and the pyrimidinediones butafenacil and saflufenacil. The visible symptoms of treatment are chlorosis and desiccation. The damage is caused by an accumulation of protoporphyrin IX in the plant cells by inhibiting protox within the tetrapyrrole biosynthesis pathway. In plants, it is encoded by two nucleus-localised homologous genes, PPOX1 and PPOX2. PPOX1 is targeted exclusively to plastids, providing Proto for heme and chlorophyll synthesis, while PPOX2 has been found in plastid envelope and mitochondria in spinach. However, N. tabacum PPOX2 was shown to be solely a mitochondrial protein.

Suitably the PPO enzyme has been modified to increase activity of the enzyme, suitably within a plant or a part thereof. Suitably the plant may have been modified to comprise a modified PPO enzyme having increased activity when compared to the unmodified, suitably wildtype PPO enzyme, suitably within the plant or a part thereof.

Suitably the activity of the modified PPO enzyme is increased to a level which provides the plant with increased resistance to a compound that inhibits PPO enzymatic activity relative to an unmodified enzyme or control enzyme.

Suitably the activity of the modified PPO enzyme is increased to a level which provides a plant with increased resistance to a compound that inhibits PPO enzymatic activity relative to an unmodified enzyme or control enzyme.

Suitably the activity of the modified PPO enzyme is increased to a level of at least 5%, at least 10%, at least 20%, at least 30%, at least 40%, at least 50%, at least 60%, at least 70%, at least 80%, at least 90%, at least 100%, at least 110%, at least 120%, at least 130%, at least 140%, at least 150% greater than the activity thereof in an unmodified or control plant when exposed to a compound that inhibits PPO enzymatic activity.

Suitably increase in the activity of an enzyme may be measured by an enzyme assay, which measures the consumption of a substrate or production of a product over time, suitably in an in vitro environment. Such assays may be spectrophotometric, fluorometric, calorimetric, chemiluminescent, light scattering or microscale thermophoresis.

Suitably the modified PPO enzyme comprises one or more modifications, suitably one or more mutations. Suitably the one or more mutations are a selection from deletions, insertions, substitutions etc. By mutation, it is meant any substitution, deletion or insertion of 1, 2, 3, 4, 5, 6, 7, 8, 9, 10 or more amino acids.

The modified PPO enzyme may be a modified PPO1 enzyme.

The modified PPO enzyme may include at least one amino acid modification at any one of amino acid positions 362, 365 and/or 479 of SEQ ID NO:1, or residues corresponding thereto.

Suitably the modified PPO enzyme may comprise an amino acid sequence having at least 30%, at least 35%, at least 40%, at least 45%, at least 50%, at least 55%, at least 60%, at least 65%, at least 70%, at least 75%, least 80%, at least 90%, at least 91%, at least 92%, at least 93%, at least 94%, at least 95%, at least 96%, at least 97%, at least 98%, at least 99% identity or 100% identity to an amino acid sequence of SEQ ID NO: 1, 2, 4-151, 153-302, or 305-336 respectively, or a functional fragment thereof. Suitably the modified PPO enzyme may comprise an amino acid sequence having at least 80%, at least 90%, at least 91%, at least 92%, at least 93%, at least 94%, at least 95%, at least 96%, at least 97%, at least 98%, at least 99% identity to an amino acid sequence of SEQ ID NO: 1, 2, 4-151, 153-302, or 305-336 respectively, or a functional fragment thereof. Suitably the modified PPO enzyme may comprise an amino acid sequence having at least 30%, at least 35%, at least 40%, at least 45%, at least 50%, at least 55%, at least 60%, at least 65%, at least 70%, at least 75%, least 80%, at least 90%, at least 91%, at least 92%, at least 93%, at least 94%, at least 95%, at least 96%, at least 97%, at least 98%, at least 99% identity or 100% identity to an amino acid sequence of SEQ ID NO: 37-39, 58, 59, 97-99, 118, 119, 125-137, 139-151, 188-190, 209, 210, 248-250, 269, 270, 277-288 or 291-302 respectively, or a functional fragment thereof. Suitably the modified PPO enzyme may comprise an amino acid sequence having at least 80%, at least 90%, at least 91%, at least 92%, at least 93%, at least 94%, at least 95%, at least 96%, at least 97%, at least 98%, at least 99% identity to an amino acid sequence of SEQ ID NO: 37-39, 58, 59, 97-99, 118, 119, 125-137, 139-151, 188-190, 209, 210, 248-250, 269, 270, 277-288 or 291-302 respectively, or a functional fragment thereof.

Suitably the modified PPO enzyme may comprise an amino acid sequence having at least 30%, at least 35%, at least 40%, at least 45%, at least 50%, at least 55%, at least 60%, at least 65%, at least 70%, at least 75%, least 80%, at least 90%, at least 91%, at least 92%, at least 93%, at least 94%, at least 95%, at least 96%, at least 97%, at least 98%, at least 99% identity or 100% identity to an amino acid sequence of SEQ ID NO: 58, 59, 126, 127, 130, 131, 132, 134, 136, 137, 209, 210, 277, 278, 281, 282, 283, 285, 287, 288, 118, 119, 140, 141, 144, 145, 146, 148, 150, 151, 269, 270, 291, 292, 295, 296, 297, 299, 301, or 302 respectively, or a functional fragment thereof. Suitably the modified PPO enzyme may comprise an amino acid sequence having at least 80%, at least 90%, at least 91%, at least 92%, at least 93%, at least 94%, at least 95%, at least 96%, at least 97%, at least 98%, at least 99% identity to an amino acid sequence of SEQ ID NO: 58, 59, 126, 127, 130, 131, 132, 134, 136, 137, 209, 210, 277, 278, 281, 282, 283, 285, 287, 288, 118, 119, 140, 141, 144, 145, 146, 148, 150, 151, 269, 270, 291, 292, 295, 296, 297, 299, 301, or 302 respectively, or a functional fragment thereof.

Suitably the modified PPO enzyme may comprise an amino acid sequence having at least 30%, at least 35%, at least 40%, at least 45%, at least 50%, at least 55%, at least 60%, at least 65%, at least 70%, at least 75%, least 80%, at least 90%, at least 91%, at least 92%, at least 93%, at least 94%, at least 95%, at least 96%, at least 97%, at least 98%, at least 99% identity or 100% identity to an amino acid sequence of SEQ ID NO: 125, 139, 276, 290, 126, 131, 134, 140, 145, 148, 277, 282, 285, 291, 296, or 299 respectively, or a functional fragment thereof. Suitably the modified PPO enzyme may comprise an amino acid sequence having at least 80%, at least 90%, at least 91%, at least 92%, at least 93%, at least 94%, at least 95%, at least 96%, at least 97%, at least 98%, at least 99% identity to an amino acid sequence of SEQ ID NO: 125, 139, 276, 290, 126, 131, 134, 140, 145, 148, 277, 282, 285, 291, 296, or 299 respectively, or a functional fragment thereof.

Suitably the modified PPO enzyme may comprise an amino acid sequence having at least 30%, at least 35%, at least 40%, at least 45%, at least 50%, at least 55%, at least 60%, at least 65%, at least 70%, at least 75%, least 80%, at least 90%, at least 91%, at least 92%, at least 93%, at least 94%, at least 95%, at least 96%, at least 97%, at least 98%, at least 99% identity or 100% identity to an amino acid sequence of SEQ ID NO: 125, 126, 127, 131, 132, 134, 276, 277, 278, 282, 283, 285, 139, 140, 141, 145, 146, 148, 290, 291, 292, 296, 297, or 299 respectively, or a functional fragment thereof. Suitably the modified PPO enzyme may comprise an amino acid sequence having at least 80%, at least 90%, at least 91%, at least 92%, at least 93%, at least 94%, at least 95%, at least 96%, at least 97%, at least 98%, at least 99% identity to an amino acid sequence of SEQ ID NO: 125, 126, 127, 131, 132, 134, 276, 277, 278, 282, 283, 285, 139, 140, 141, 145, 146, 148, 290, 291, 292, 296, 297, or 299 respectively, or a functional fragment thereof. Suitably the modified PPO enzyme may comprise an amino acid sequence having at least 30%, at least 35%, at least 40%, at least 45%, at least 50%, at least 55%, at least 60%, at least 65%, at least 70%, at least 75%, least 80%, at least 90%, at least 91%, at least 92%, at least 93%, at least 94%, at least 95%, at least 96%, at least 97%, at least 98%, at least 99% identity or 100% identity to an amino acid sequence of SEQ ID NO: 125, 131, 134, 139, 145, 148, 276, 282, 285, 290, 296 or 299 respectively, or a functional fragment thereof. Suitably the modified PPO enzyme may comprise an amino acid sequence having at least 80%, at least 90%, at least 91%, at least 92%, at least 93%, at least 94%, at least 95%, at least 96%, at least 97%, at least 98%, at least 99% identity to an amino acid sequence of SEQ ID NO: 125, 131, 134, 139, 145, 148, 276, 282, 285, 290, 296 or 299 respectively, or a functional fragment thereof.

Suitably the modified PPO enzyme may comprise an amino acid sequence having at least 30%, at least 35%, at least 40%, at least 45%, at least 50%, at least 55%, at least 60%, at least 65%, at least 70%, at least 75%, least 80%, at least 90%, at least 91%, at least 92%, at least 93%, at least 94%, at least 95%, at least 96%, at least 97%, at least 98%, at least 99% identity or 100% identity to an amino acid sequence of SEQ ID NO: 125, 139, 276, or 290, respectively, or a functional fragment thereof. Suitably the modified PPO enzyme may comprise an amino acid sequence having at least 80%, at least 90%, at least 91%, at least 92%, at least 93%, at least 94%, at least 95%, at least 96%, at least 97%, at least 98%, at least 99% identity to an amino acid sequence of SEQ ID NO: 125, 139, 276, or 290 respectively, or a functional fragment thereof.

Suitably the modified PPO enzyme may comprise an amino acid sequence having at least 30%, at least 35%, at least 40%, at least 45%, at least 50%, at least 55%, at least 60%, at least 65%, at least 70%, at least 75%, least 80%, at least 90%, at least 91%, at least 92%, at least 93%, at least 94%, at least 95%, at least 96%, at least 97%, at least 98%, at least 99% identity or 100% identity to an amino acid sequence of SEQ ID NO: 131, 145, 282, or 296, respectively, or a functional fragment thereof. Suitably the modified PPO enzyme may comprise an amino acid sequence having at least 80%, at least 90%, at least 91%, at least 92%, at least 93%, at least 94%, at least 95%, at least 96%, at least 97%, at least 98%, at least 99% identity to an amino acid sequence of SEQ ID NO: 131, 145, 282, or 296 respectively, or a functional fragment thereof.

Suitably the modified PPO enzyme may comprise an amino acid sequence having at least 30%, at least 35%, at least 40%, at least 45%, at least 50%, at least 55%, at least 60%, at least 65%, at least 70%, at least 75%, least 80%, at least 90%, at least 91%, at least 92%, at least 93%, at least 94%, at least 95%, at least 96%, at least 97%, at least 98%, at least 99% identity or 100% identity to an amino acid sequence of SEQ ID NO: 126, 140, 277, or 291, respectively, or a functional fragment thereof. Suitably the modified PPO enzyme may comprise an amino acid sequence having at least 80%, at least 90%, at least 91%, at least 92%, at least 93%, at least 94%, at least 95%, at least 96%, at least 97%, at least 98%, at least 99% identity to an amino acid sequence of SEQ ID NO: 126, 140, 277, or 291 respectively, or a functional fragment thereof.

Suitably the modified PPO enzyme may comprise an amino acid sequence having at least 30%, at least 35%, at least 40%, at least 45%, at least 50%, at least 55%, at least 60%, at least 65%, at least 70%, at least 75%, least 80%, at least 90%, at least 91%, at least 92%, at least 93%, at least 94%, at least 95%, at least 96%, at least 97%, at least 98%, at least 99% identity or 100% identity to an amino acid sequence of SEQ ID NO: 134, 148, 285, or 299, respectively, or a functional fragment thereof. Suitably the modified PPO enzyme may comprise an amino acid sequence having at least 80%, at least 90%, at least 91%, at least 92%, at least 93%, at least 94%, at least 95%, at least 96%, at least 97%, at least 98%, at least 99% identity to an amino acid sequence of SEQ ID NO: 134, 148, 285, or 299 respectively, or a functional fragment thereof.

Suitably the modified PPO enzyme may comprise an amino acid sequence having at least 30%, at least 35%, at least 40%, at least 45%, at least 50%, at least 55%, at least 60%, at least 65%, at least 70%, at least 75%, least 80%, at least 90%, at least 91%, at least 92%, at least 93%, at least 94%, at least 95%, at least 96%, at least 97%, at least 98%, at least 99% identity or 100% identity to an amino acid sequence of SEQ ID NO: 132, 283, 146 or 297, respectively, or a functional fragment thereof. Suitably the modified PPO enzyme may comprise an amino acid sequence having at least 80%, at least 90%, at least 91%, at least 92%, at least 93%, at least 94%, at least 95%, at least 96%, at least 97%, at least 98%, at least 99% identity to an amino acid sequence of SEQ ID NO: 132, 283, 146 or 297 respectively, or a functional fragment thereof.

Suitably the modified PPO enzyme may comprise an amino acid sequence having at least 30%, at least 35%, at least 40%, at least 45%, at least 50%, at least 55%, at least 60%, at least 65%, at least 70%, at least 75%, least 80%, at least 90%, at least 91%, at least 92%, at least 93%, at least 94%, at least 95%, at least 96%, at least 97%, at least 98%, at least 99% identity or 100% identity to an amino acid sequence of SEQ ID NO: 127, 278, 141 or 292, respectively, or a functional fragment thereof. Suitably the modified PPO enzyme may comprise an amino acid sequence having at least 80%, at least 90%, at least 91%, at least 92%, at least 93%, at least 94%, at least 95%, at least 96%, at least 97%, at least 98%, at least 99% identity to an amino acid sequence of SEQ ID NO: 127, 278, 141 or 292 respectively, or a functional fragment thereof.

In one embodiment, the modified PPO enzyme may be derived from Arabidopsis thaliana. In one embodiment, the modified PPO enzyme may be derived from Setaria italica.

A modified PPO enzyme of the invention may comprise an amino acid sequence according to SEQ ID NO:1 (Arabidopsis thaliana PPO) wherein positions 362, 365 and/or 479 or residues corresponding thereto have been modified.

A modified PPO enzyme of the invention may comprise an amino acid sequence according to SEQ ID NO:2 (Setaria italica PPO) wherein positions 360, 363 and/or 477 or residues corresponding thereto have been modified. Positions 360, 363 and 477 respectively correspond to positions 362, 365 and 479 of SEQ ID NO: 1.

A modified PPO enzyme of the invention may comprise an amino acid sequence according to SEQ ID NO:1 (Arabidopsis thaliana PPO) wherein positions 305 and/or 426 or residues corresponding thereto have been modified, suitably to be leucine and valine respectively.

A modified PPO enzyme of the invention may comprise an amino acid sequence according to SEQ ID NO:2 (Setaria italica PPO) wherein positions 303 and/or 424 or residues corresponding thereto have been modified, suitably to be leucine and valine respectively.

For example, a modified PPO enzyme of the invention may comprise an amino acid sequence according to SEQ ID NO:1 (Arabidopsis thaliana PPO) wherein position 362 has been modified.

For example, a modified PPO enzyme of the invention may comprise an amino acid sequence according to SEQ ID NO:1 (Arabidopsis thaliana PPO) wherein position 365 has been modified.

For example, a modified PPO enzyme of the invention may comprise an amino acid sequence according to SEQ ID NO:1 (Arabidopsis thaliana PPO) wherein position 479 has been modified.

For example, a modified PPO enzyme of the invention may comprise an amino acid sequence according to SEQ ID NO:2 (Setaria italica PPO) wherein position 360 has been modified.

For example, a modified PPO enzyme of the invention may comprise an amino acid sequence according to SEQ ID NO:2 (Setaria italica PPO) wherein position 363 has been modified.

For example, a modified PPO enzyme of the invention may comprise an amino acid sequence according to SEQ ID NO:2 (Setaria italica PPO) wherein position 477 has been modified.

Suitably, a modified PPO of the invention may comprise an amino acid sequence of at least 30%, at least 35%, at least 40%, at least 45%, at least 50%, at least 55%, at least 60%, at least 70%, at least 75%, at least 80%, at least 85%, at least 90%, at least 91%, at least 92%, at least 93%, at least 94%, at least 95%, at least 98% or at least 99% sequence identity to an amino acid sequence according to SEQ ID NO: 1, wherein at least one of positions 362, 365 and 479 thereof, or corresponding positions thereto, have been modified.

Suitably, a modified PPO of the invention may comprise an amino acid sequence of at least 30%, at least 35%, at least 40%, at least 45%, at least 50%, at least 55%, at least 60%, at least 70%, at least 75%, at least 80%, at least 85%, at least 90%, at least 91%, at least 92%, at least 93%, at least 94%, at least 95%, at least 98% or at least 99% sequence identity to an amino acid sequence according to SEQ ID NO: 2, wherein at least one of positions 360, 363 and 477 thereof, or corresponding positions thereto, have been modified.

Suitably, a modified PPO of the invention may comprise one or more amino acid sequence motifs as set out in Table 27

Suitably, position 362 of SEQ ID NO: 1 or corresponding positions thereto may be substituted with a cysteine. In some examples, position 362 of SEQ ID NO: 1 or corresponding positions thereto may be substituted with a phenylalanine. Suitably, position 360 of SEQ ID NO: 2 or corresponding positions thereto may be substituted with a cysteine.

In some examples, position 360 of SEQ ID NO: 2 or corresponding positions thereto may be substituted with a phenylalanine.

Suitably, position 365 of SEQ ID NO: 1 or corresponding positions thereto may be substituted with a methionine. In some examples, position 365 of SEQ ID NO: 1 or corresponding positions thereto may be substituted with a leucine. Suitably, position 363 of SEQ ID NO: 2 or corresponding positions thereto may be substituted with a methionine. In some examples, position 363 of SEQ ID NO: 2 or corresponding positions thereto may be substituted with a leucine.

Suitably, position 479 of SEQ ID NO: 1 or corresponding positions thereto may be substituted with a methionine. In some examples, position 479 of SEQ ID NO: 1 or corresponding positions thereto may be substituted with an asparagine. Suitably, position 477 of SEQ ID NO: 2 or corresponding positions thereto may be substituted with a methionine. In some examples, position 477 of SEQ ID NO: 2 or corresponding positions thereto may be substituted with an asparagine.

In some examples, the modified PPOs of the invention may include one or more additional modifications in addition to those detailed above. For example, modification of any one of the amino acid residues 305, 361, 404, 426, 431 and/or 461 of SEQ ID NO: 1 or residues corresponding thereto.

Suitably, residue 305 of SEQ ID NO: 1 or a residue corresponding thereto may be substituted with leucine.

Suitably, residue 361 of SEQ ID NO: 1 or a residue corresponding thereto may be substituted with threonine. Suitably, residue 361 of SEQ ID NO: 1 or a residue corresponding thereto may be substituted with cysteine. Suitably, residue 361 of SEQ ID NO: 1 or a residue corresponding thereto may be substituted with aspartic acid. Suitably, residue 361 of SEQ ID NO: 1 or a residue corresponding thereto may be substituted with glutamine.

Suitably, residue 404 of SEQ ID NO: 1 or a residue corresponding thereto may be substituted with alanine.

Suitably, residue 426 of SEQ ID NO: 1 or a residue corresponding thereto may be substituted with leucine. Suitably, residue 426 of SEQ ID NO: 1 or a residue corresponding thereto may be substituted with cysteine. Suitably, residue 426 of SEQ ID NO: 1 or a residue corresponding thereto may be substituted with threonine. Suitably, residue 426 of SEQ ID NO: 1 or a residue corresponding thereto may be substituted with valine. Suitably, residue 426 of SEQ ID NO: 1 or a residue corresponding thereto may be substituted with methionine.

Suitably, residue 431 of SEQ ID NO: 1 or a residue corresponding thereto may be substituted with phenylalanine. Suitably, residue 431 of SEQ ID NO: 1 or a residue corresponding thereto may be substituted with alanine. Suitably, residue 431 of SEQ ID NO: 1 or a residue corresponding thereto may be substituted with arginine.

Suitably, residue 461 of SEQ ID NO: 1 or a residue corresponding thereto may be substituted with glutamine.

For example, modification of any one of the amino acid residues 303, 359, 402, 424, 429 and/or 459 of SEQ ID NO: 2 or residues corresponding thereto.

Suitably, residue 303 of SEQ ID NO: 2 or a residue corresponding thereto may be substituted with leucine.

Suitably, residue 359 of SEQ ID NO: 2 or a residue corresponding thereto may be substituted with threonine. Suitably, residue 359 of SEQ ID NO: 2 or a residue corresponding thereto may be substituted with cysteine. Suitably, residue 359 of SEQ ID NO: 2 or a residue corresponding thereto may be substituted with aspartic acid. Suitably, residue 359 of SEQ ID NO: 2 or a residue corresponding thereto may be substituted with glutamine.

Suitably, residue 402 of SEQ ID NO: 2 or a residue corresponding thereto may be substituted with alanine.

Suitably, residue 424 of SEQ ID NO: 2 or a residue corresponding thereto may be substituted with leucine. Suitably, residue 424 of SEQ ID NO: 2 or a residue corresponding thereto may be substituted with cysteine. Suitably, residue 424 of SEQ ID NO: 2 or a residue corresponding thereto may be substituted with threonine. Suitably, residue 424 of SEQ ID NO: 2 or a residue corresponding thereto may be substituted with valine. Suitably, residue 424 of SEQ ID NO: 2 or a residue corresponding thereto may be substituted with methionine.

Suitably, residue 429 of SEQ ID NO: 2 or a residue corresponding thereto may be substituted with phenylalanine. Suitably, residue 429 of SEQ ID NO: 2 or a residue corresponding thereto may be substituted with asparagine.

Suitably, residue 459 of SEQ ID NO: 2 or a residue corresponding thereto may be substituted with glutamine.

In some examples, the modified PPO enzyme may include modifications at amino acid residue positions 365, 426, and 479 of SEQ ID NO: 1 or residues corresponding thereto.

In some examples, the modified PPO enzyme may include modifications at amino acid residue positions 305, 404, 426, 431, and 479 of SEQ ID NO: 1 or residues corresponding thereto.

In some examples, the modified PPO enzyme may include modifications at amino acid residue positions 305 and 365 of SEQ ID NO: 1 or residues corresponding thereto.

In some examples, the modified PPO enzyme may include modifications at amino acid residue positions 305, 362 and 404 of SEQ ID NO: 1 or residues corresponding thereto.

In some examples, the modified PPO enzyme may include modifications at amino acid residue positions 305, 361, 365, 431 and 479 of SEQ ID NO: 1 or residues corresponding thereto.

In some examples, the modified PPO enzyme may include modifications at amino acid residue positions 305, 426 and 479 of SEQ ID NO: 1 or residues corresponding thereto.

In some examples, the modified PPO enzyme may include modifications at amino acid residue positions 426, 461 and 479 of SEQ ID NO: 1 or residues corresponding thereto.

In some examples, the modified PPO enzyme may include modifications at amino acid residue positions 305, 361 and 365 of SEQ ID NO: 1 or residues corresponding thereto.

In some examples, the modified PPO enzyme may include modifications at amino acid residue positions 305, 361, 362, 426 and 479 of SEQ ID NO: 1 or residues corresponding thereto.

In some examples, the modified PPO enzyme may include modifications at amino acid residue positions 361 and 365 of SEQ ID NO: 1 or residues corresponding thereto.

In some examples, the modified PPO enzyme may include modifications at amino acid residue positions 365, 404, and 479 of SEQ ID NO: 1 or residues corresponding thereto.

In some examples, the modified PPO enzyme may include modifications at amino acid residue positions 305, 404, and 479 of SEQ ID NO: 1 or residues corresponding thereto.

In some examples, the modified PPO enzyme may include modifications at amino acid residues positions 305 and 426 of SEQ ID NO: 1 or residues corresponding thereto.

In some examples, the modified PPO enzyme may include modifications at amino acid residue positions 363, 424, and 477 of SEQ ID NO: 2 or residues corresponding thereto.

In some examples, the modified PPO enzyme may include modifications at amino acid residue positions 303, 402, 424, 429, and 477 of SEQ ID NO: 2 or residues corresponding thereto.

In some examples, the modified PPO enzyme may include modifications at amino acid residue positions 303 and 363 of SEQ ID NO: 2 or residues corresponding thereto.

In some examples, the modified PPO enzyme may include modifications at amino acid residue positions 303, 360 and 402 of SEQ ID NO: 2 or residues corresponding thereto.

In some examples, the modified PPO enzyme may include modifications at amino acid residue positions 303, 359, 363, 429 and 477 of SEQ ID NO: 2 or residues corresponding thereto.

In some examples, the modified PPO enzyme may include modifications at amino acid residue positions 303, 424 and 477 of SEQ ID NO: 2 or residues corresponding thereto.

In some examples, the modified PPO enzyme may include modifications at amino acid residue positions 424, 459 and 477 of SEQ ID NO: 2 or residues corresponding thereto.

In some examples, the modified PPO enzyme may include modifications at amino acid residue positions 303, 359 and 363 of SEQ ID NO: 2 or residues corresponding thereto.

In some examples, the modified PPO enzyme may include modifications at amino acid residue positions 303, 359, 360, 424 and 477 of SEQ ID NO: 2 or residues corresponding thereto.

In some examples, the modified PPO enzyme may include modifications at amino acid residue positions 359 and 363 of SEQ ID NO: 2 or residues corresponding thereto.

In some examples, the modified PPO enzyme may include modifications at amino acid residue positions 363, 402, and 477 of SEQ ID NO: 2 or residues corresponding thereto.

In some examples, the modified PPO enzyme may include modifications at amino acid residue positions 303, 402, and 477 of SEQ ID NO: 2 or residues corresponding thereto.

In some examples, the modified PPO enzyme may include modifications at amino acid residues positions 303 and 424 of SEQ ID NO: 2 or residues corresponding thereto.

Suitably, the modified PPO may have an amino acid sequence having at least 30% sequence identity to SEQ ID NO: 37 (wherein the amino acid sequence includes the substitution Y362F compared to SEQ ID NO:1). Suitably, the modified PPO may have an amino acid sequence having at least 50% sequence identity to SEQ ID NO: 37. Suitably, the modified PPO may have an amino acid sequence having at least 85%, 90%, 91%, 92%, 93%, 94%, 95%, 96%, 97%, 98%, 99% or 100% sequence identity to SEQ ID NO: 37.

Suitably, the modified PPO may comprise an amino acid sequence according to SEQ ID NO: 37. Suitably, the modified PPO may essentially consist of an amino acid sequence according to SEQ ID NO: 37.

Suitably, the modified PPO may have an amino acid sequence having at least 30% sequence identity to SEQ ID NO: 38 (wherein the amino acid sequence includes the substitution V365L compared to SEQ ID NO:1). Suitably, the modified PPO may have an amino acid sequence having at least 50% sequence identity to SEQ ID NO: 38. Suitably, the modified PPO may have an amino acid sequence having at least 85%, 90%, 91%, 92%, 93%, 94%, 95%, 96%, 97%, 98%, 99% or 100% sequence identity to SEQ ID NO: 38.

Suitably, the modified PPO may comprise an amino acid sequence according to SEQ ID NO: 38. Suitably, the modified PPO may essentially consist of an amino acid sequence according to SEQ ID NO: 38.

Suitably, the modified PPO may have an amino acid sequence having at least 30% sequence identity to SEQ ID NO: 39 (wherein the amino acid sequence includes the substitution V365M compared to SEQ ID NO:1). Suitably, the modified PPO may have an amino acid sequence having at least 50% sequence identity to SEQ ID NO: 39. Suitably, the modified PPO may have an amino acid sequence having at least 85%, 90%, 91%, 92%, 93%, 94%, 95%, 96%, 97%, 98%, 99% or 100% sequence identity to SEQ ID NO: 39.

Suitably, the modified PPO may comprise an amino acid sequence according to SEQ ID NO: 39. Suitably, the modified PPO may essentially consist of an amino acid sequence according to SEQ ID NO: 39.

Suitably, the modified PPO may have an amino acid sequence having at least 30% sequence identity to SEQ ID NO: 58 (wherein the amino acid sequence includes the substitution L479M compared to SEQ ID NO:1). Suitably, the modified PPO may have an amino acid sequence having at least 50% sequence identity to SEQ ID NO: 58. Suitably, the modified PPO may have an amino acid sequence having at least 85%, 90%, 91%, 92%, 93%, 94%, 95%, 96%, 97%, 98%, 99% or 100% sequence identity to SEQ ID NO: 58.

Suitably, the modified PPO may comprise an amino acid sequence according to SEQ ID NO: 58. Suitably, the modified PPO may essentially consist of an amino acid sequence according to SEQ ID NO: 58.

Suitably, the modified PPO may have an amino acid sequence having at least 30% sequence identity to SEQ ID NO: 59 (wherein the amino acid sequence includes the substitution L479N compared to SEQ ID NO:1). Suitably, the modified PPO may have an amino acid sequence having at least 50% sequence identity to SEQ ID NO: 59. Suitably, the modified PPO may have an amino acid sequence having at least 85%, 90%, 91%, 92%, 93%, 94%, 95%, 96%, 97%, 98%, 99% or 100% sequence identity to SEQ ID NO: 59.

Suitably, the modified PPO may comprise an amino acid sequence according to SEQ ID NO: 59. Suitably, the modified PPO may essentially consist of an amino acid sequence according to SEQ ID NO: 59.

Suitably, the modified PPO may have an amino acid sequence having at least 30% sequence identity to SEQ ID NO: 97 (wherein the amino acid sequence includes the substitution Y360F compared to SEQ ID NO:2). Suitably, the modified PPO may have an amino acid sequence having at least 50% sequence identity to SEQ ID NO: 97. Suitably, the modified PPO may have an amino acid sequence having at least 85%, 90%, 91%, 92%, 93%, 94%, 95%, 96%, 97%, 98%, 99% or 100% sequence identity to SEQ ID NO: 97.

Suitably, the modified PPO may comprise an amino acid sequence according to SEQ ID NO: 97. Suitably, the modified PPO may essentially consist of an amino acid sequence according to SEQ ID NO: 97.

Suitably, the modified PPO may have an amino acid sequence having at least 30% sequence identity to SEQ ID NO: 98 (wherein the amino acid sequence includes the substitution V363L compared to SEQ ID NO:2). Suitably, the modified PPO may have an amino acid sequence having at least 50% sequence identity to SEQ ID NO: 98. Suitably, the modified PPO may have an amino acid sequence having at least 85%, 90%, 91%, 92%, 93%, 94%, 95%, 96%, 97%, 98%, 99% or 100% sequence identity to SEQ ID NO: 98.

Suitably, the modified PPO may comprise an amino acid sequence according to SEQ ID NO: 98. Suitably, the modified PPO may essentially consist of an amino acid sequence according to SEQ ID NO: 98.

Suitably, the modified PPO may have an amino acid sequence having at least 30% sequence identity to SEQ ID NO: 99 (wherein the amino acid sequence includes the substitution V363M compared to SEQ ID NO:2). Suitably, the modified PPO may have an amino acid sequence having at least 50% sequence identity to SEQ ID NO: 99. Suitably, the modified PPO may have an amino acid sequence having at least 85%, 90%, 91%, 92%, 93%, 94%, 95%, 96%, 97%, 98%, 99% or 100% sequence identity to SEQ ID NO: 99.

Suitably, the modified PPO may comprise an amino acid sequence according to SEQ ID NO: 99. Suitably, the modified PPO may essentially consist of an amino acid sequence according to SEQ ID NO: 99.

Suitably, the modified PPO may have an amino acid sequence having at least 30% sequence identity to SEQ ID NO: 118 (wherein the amino acid sequence includes the substitution V477M compared to SEQ ID NO:2). Suitably, the modified PPO may have an amino acid sequence having at least 50% sequence identity to SEQ ID NO: 118. Suitably, the modified PPO may have an amino acid sequence having at least 85%, 90%, 91%, 92%, 93%, 94%, 95%, 96%, 97%, 98%, 99% or 100% sequence identity to SEQ ID NO: 118.

Suitably, the modified PPO may comprise an amino acid sequence according to SEQ ID NO: 118. Suitably, the modified PPO may essentially consist of an amino acid sequence according to SEQ ID NO: 118.

Suitably, the modified PPO may have an amino acid sequence having at least 30% sequence identity to SEQ ID NO: 119 (wherein the amino acid sequence includes the substitution V477N compared to SEQ ID NO:2). Suitably, the modified PPO may have an amino acid sequence having at least 50% sequence identity to SEQ ID NO: 119. Suitably, the modified PPO may have an amino acid sequence having at least 85%, 90%, 91%, 92%, 93%, 94%, 95%, 96%, 97%, 98%, 99% or 100% sequence identity to SEQ ID NO: 119.

Suitably, the modified PPO may comprise an amino acid sequence according to SEQ ID NO: 119. Suitably, the modified PPO may essentially consist of an amino acid sequence according to SEQ ID NO: 119.

Suitably, the modified PPO may have an amino acid sequence having at least 30% sequence identity to SEQ ID NO: 125 (wherein the amino acid sequence includes the substitutions S305L and Y426V compared to SEQ ID NO:1). Suitably, the modified PPO may have an amino acid sequence having at least 50% sequence identity to SEQ ID NO: 125. Suitably, the modified PPO may have an amino acid sequence having at least 85%, 90%, 91%, 92%, 93%, 94%, 95%, 96%, 97%, 98%, 99% or 100% sequence identity to SEQ ID NO: 125. Suitably, the modified PPO may comprise an amino acid sequence according to SEQ ID NO: 125. Suitably, the modified PPO may essentially consist of an amino acid sequence according to SEQ ID NO: 125.

Suitably, the modified PPO may have an amino acid sequence having at least 30% sequence identity to SEQ ID NO: 126 (wherein the amino acid sequence includes the substitutions V365L, Y426V, and L479N compared to SEQ ID NO:1). Suitably, the modified PPO may have an amino acid sequence having at least 50% sequence identity to SEQ ID NO: 126. Suitably, the modified PPO may have an amino acid sequence having at least 85% 90%, 91%, 92%, 93%, 94%, 95%, 96%, 97%, 98%, 99% or 100% sequence identity to SEQ ID NO: 126. Suitably, the modified PPO may comprise an amino acid sequence according to SEQ ID NO: 126. Suitably, the modified PPO may essentially consist of an amino acid sequence according to SEQ ID NO: 126.

Suitably, the modified PPO may have an amino acid sequence having at least 30% sequence identity to SEQ ID NO: 127 (wherein the amino acid sequence includes the substitution S305L, G404A, Y426V, T431R, and L479N compared to SEQ ID NO:1).

Suitably, the modified PPO may have an amino acid sequence having at least 50% sequence identity to SEQ ID NO: 127. Suitably, the modified PPO may have an amino acid sequence having at least 85%, 90%, 91%, 92%, 93%, 94%, 95%, 96%, 97%, 98%, 99% or 100% sequence identity to SEQ ID NO: 127. Suitably, the modified PPO may comprise an amino acid sequence according to SEQ ID NO: 127. Suitably, the modified PPO may essentially consist of an amino acid sequence according to SEQ ID NO: 127.

Suitably, the modified PPO may have an amino acid sequence having at least 30% sequence identity to SEQ ID NO: 128 (wherein the amino acid sequence includes the substitution S305L and V365M compared to SEQ ID NO:1). Suitably, the modified PPO may have an amino acid sequence having at least 50% sequence identity to SEQ ID NO: 128.

Suitably, the modified PPO may have an amino acid sequence having at least 85%, 90%, 91%, 92%, 93%, 94%, 95%, 96%, 97%, 98%, 99% or 100% sequence identity to SEQ ID NO: 128. Suitably, the modified PPO may comprise an amino acid sequence according to SEQ ID NO: 128. Suitably, the modified PPO may essentially consist of an amino acid sequence according to SEQ ID NO: 128.

Suitably, the modified PPO may have an amino acid sequence having at least 30% sequence identity to SEQ ID NO: 129 (wherein the amino acid sequence includes the substitution S305L, Y362F, and G404A compared to SEQ ID NO:1). Suitably, the modified PPO may have an amino acid sequence having at least 50% sequence identity to SEQ ID NO: 129. the modified PPO may have an amino acid sequence having at least 85%, 90%, 91%, 92%, 93%, 94%, 95%, 96%, 97%, 98%, 99% or 100% sequence identity to SEQ ID NO: 129. Suitably, the modified PPO may comprise an amino acid sequence according to SEQ ID NO: 129. Suitably, the modified PPO may essentially consist of an amino acid sequence according to SEQ ID NO: 129.

Suitably, the modified PPO may have an amino acid sequence having at least 30% sequence identity to SEQ ID NO: 130 (wherein the amino acid sequence includes the substitution S305L, Y361T, V365L, T431R, and L479M compared to SEQ ID NO:1).

Suitably, the modified PPO may have an amino acid sequence having at least 50% sequence identity to SEQ ID NO: 130. Suitably, the modified PPO may have an amino acid sequence having at least 85%, 90%, 91%, 92%, 93%, 94%, 95%, 96%, 97%, 98%, 99% or 100% sequence identity to SEQ ID NO: 130. Suitably, the modified PPO may comprise an amino acid sequence according to SEQ ID NO: 130. Suitably, the modified PPO may essentially consist of an amino acid sequence according to SEQ ID NO: 130.

Suitably, the modified PPO may have an amino acid sequence having at least 30% sequence identity to SEQ ID NO: 131 (wherein the amino acid sequence includes the substitution S305L, Y426V, and L479M compared to SEQ ID NO:1). Suitably, the modified PPO may have an amino acid sequence having at least 50% sequence identity to SEQ ID NO: 131. Suitably, the modified PPO may have an amino acid sequence having at least 85% 90%, 91%, 92%, 93%, 94%, 95%, 96%, 97%, 98%, 99% or 100% sequence identity to SEQ ID NO: 131. Suitably, the modified PPO may comprise an amino acid sequence according to SEQ ID NO: 131. Suitably, the modified PPO may essentially consist of an amino acid sequence according to SEQ ID NO: 131.

Suitably, the modified PPO may have an amino acid sequence having at least 30% sequence identity to SEQ ID NO: 132 (wherein the amino acid sequence includes the substitution Y426M, T461Q, and L479M compared to SEQ ID NO:1). Suitably, the modified PPO may have an amino acid sequence having at least 50% sequence identity to SEQ ID NO: 132. Suitably, the modified PPO may have an amino acid sequence having at least 85%, 90%, 91%, 92%, 93%, 94%, 95%, 96%, 97%, 98%, 99% or 100% sequence identity to SEQ ID NO: 132. Suitably, the modified PPO may comprise an amino acid sequence according to SEQ ID NO: 132. Suitably, the modified PPO may essentially consist of an amino acid sequence according to SEQ ID NO: 132.

Suitably, the modified PPO may have an amino acid sequence having at least 30% sequence identity to SEQ ID NO: 133 (wherein the amino acid sequence includes the substitution S305L, Y361D, and V365M compared to SEQ ID NO:1). Suitably, the modified PPO may have an amino acid sequence having at least 50% sequence identity to SEQ ID NO: 133. Suitably, the modified PPO may have an amino acid sequence having at least 85%, 90%, 91%, 92%, 93%, 94%, 95%, 96%, 97%, 98%, 99% or 100% sequence identity to SEQ ID NO: 133. Suitably, the modified PPO may comprise an amino acid sequence according to SEQ ID NO: 133. Suitably, the modified PPO may essentially consist of an amino acid sequence according to SEQ ID NO: 133.

Suitably, the modified PPO may have an amino acid sequence having at least 30% sequence identity to SEQ ID NO: 134 (wherein the amino acid sequence includes the substitution S305L, Y361C, Y362F, Y426M, and L479M compared to SEQ ID NO:1).

Suitably, the modified PPO may have an amino acid sequence having at least 50% sequence identity to SEQ ID NO: 134. Suitably, the modified PPO may have an amino acid sequence having at least 85%, 90%, 91%, 92%, 93%, 94%, 95%, 96%, 97%, 98%, 99% or 100% sequence identity to SEQ ID NO: 134. Suitably, the modified PPO may comprise an amino acid sequence according to SEQ ID NO: 134. Suitably, the modified PPO may essentially consist of an amino acid sequence according to SEQ ID NO: 134.

Suitably, the modified PPO may have an amino acid sequence having at least 30% sequence identity to SEQ ID NO: 135 (wherein the amino acid sequence includes the substitution Y361D and V365L compared to SEQ ID NO:1). Suitably, the modified PPO may have an amino acid sequence having at least 50% sequence identity to SEQ ID NO: 135.

Suitably, the modified PPO may have an amino acid sequence having at least 85%, 90%, 91%, 92%, 93%, 94%, 95%, 96%, 97%, 98%, 99% or 100% sequence identity to SEQ ID NO: 135. Suitably, the modified PPO may comprise an amino acid sequence according to SEQ ID NO: 135. Suitably, the modified PPO may essentially consist of an amino acid sequence according to SEQ ID NO: 135.

Suitably, the modified PPO may have an amino acid sequence having at least 30% sequence identity to SEQ ID NO: 136 (wherein the amino acid sequence includes the substitution V365L, G404A, and L479N compared to SEQ ID NO:1). Suitably, the modified PPO may have an amino acid sequence having at least 50% sequence identity to SEQ ID NO: 136. Suitably, the modified PPO may have an amino acid sequence having at least 85%, 90%, 91%, 92%, 93%, 94%, 95%, 96%, 97%, 98%, 99% or 100% sequence identity to SEQ ID NO: 136. Suitably, the modified PPO may comprise an amino acid sequence according to SEQ ID NO: 136. Suitably, the modified PPO may essentially consist of an amino acid sequence according to SEQ ID NO: 136.

Suitably, the modified PPO may have an amino acid sequence having at least 30% sequence identity to SEQ ID NO: 137 (wherein the amino acid sequence includes the substitution S305L, G404A, and L479N compared to SEQ ID NO:1). Suitably, the modified PPO may have an amino acid sequence having at least 50% sequence identity to SEQ ID NO: 137. Suitably, the modified PPO may have an amino acid sequence having at least 85% 90%, 91%, 92%, 93%, 94%, 95%, 96%, 97%, 98%, 99% or 100% sequence identity to SEQ ID NO: 137. Suitably, the modified PPO may comprise an amino acid sequence according to SEQ ID NO: 137. Suitably, the modified PPO may essentially consist of an amino acid sequence according to SEQ ID NO: 137.

Suitably, the modified PPO may have an amino acid sequence having at least 30% sequence identity to SEQ ID NO: 138 (wherein the amino acid sequence includes the substitution T303L and Y424M compared to SEQ ID NO:2). Suitably, the modified PPO may have an amino acid sequence having at least 50% sequence identity to SEQ ID NO: 138.

Suitably, the modified PPO may have an amino acid sequence having at least 85%, 90%, 91%, 92%, 93%, 94%, 95%, 96%, 97%, 98%, 99% or 100% sequence identity to SEQ ID NO: 138. Suitably, the modified PPO may comprise an amino acid sequence according to SEQ ID NO: 138. Suitably, the modified PPO may essentially consist of an amino acid sequence according to SEQ ID NO: 138.

Suitably, the modified PPO may have an amino acid sequence having at least 30% sequence identity to SEQ ID NO: 139 (wherein the amino acid sequence includes the substitution T303L and Y424V compared to SEQ ID NO:2). Suitably, the modified PPO may have an amino acid sequence having at least 50% sequence identity to SEQ ID NO: 139.

Suitably, the modified PPO may have an amino acid sequence having at least 85%, 90%, 91%, 92%, 93%, 94%, 95%, 96%, 97%, 98%, 99% or 100% sequence identity to SEQ ID NO: 139. Suitably, the modified PPO may comprise an amino acid sequence according to SEQ ID NO: 139. Suitably, the modified PPO may essentially consist of an amino acid sequence according to SEQ ID NO: 139.

Suitably, the modified PPO may have an amino acid sequence having at least 30% sequence identity to SEQ ID NO: 140 (wherein the amino acid sequence includes the substitution V363L, Y424V, and L477N compared to SEQ ID NO:2). Suitably, the modified PPO may have an amino acid sequence having at least 50% sequence identity to SEQ ID NO: 140. Suitably, the modified PPO may have an amino acid sequence having at least 85%, 90%, 91%, 92%, 93%, 94%, 95%, 96%, 97%, 98%, 99% or 100% sequence identity to SEQ ID NO: 140. Suitably, the modified PPO may comprise an amino acid sequence according to SEQ ID NO: 140. Suitably, the modified PPO may essentially consist of an amino acid sequence according to SEQ ID NO: 140.

Suitably, the modified PPO may have an amino acid sequence having at least 30% sequence identity to SEQ ID NO: 141 (wherein the amino acid sequence includes the substitution T303L, G402A, Y424V, T429R, and L477N compared to SEQ ID NO:2).

Suitably, the modified PPO may have an amino acid sequence having at least 50% sequence identity to SEQ ID NO: 141. Suitably, the modified PPO may have an amino acid sequence having at least 85%, 90%, 91%, 92%, 93%, 94%, 95%, 96%, 97%, 98%, 99% or 100% sequence identity to SEQ ID NO: 141. Suitably, the modified PPO may comprise an amino acid sequence according to SEQ ID NO: 141. Suitably, the modified PPO may essentially consist of an amino acid sequence according to SEQ ID NO: 141.

Suitably, the modified PPO may have an amino acid sequence having at least 30% sequence identity to SEQ ID NO: 142 (wherein the amino acid sequence includes the substitution T303L and V363M compared to SEQ ID NO:2). Suitably, the modified PPO may have an amino acid sequence having at least 50% sequence identity to SEQ ID NO: 142.

Suitably, the modified PPO may have an amino acid sequence having at least 85%, 90%, 91%, 92%, 93%, 94%, 95%, 96%, 97%, 98%, 99% or 100% sequence identity to SEQ ID NO: 142. Suitably, the modified PPO may comprise an amino acid sequence according to SEQ ID NO: 142. Suitably, the modified PPO may essentially consist of an amino acid sequence according to SEQ ID NO: 142.

Suitably, the modified PPO may have an amino acid sequence having at least 30% sequence identity to SEQ ID NO: 143 (wherein the amino acid sequence includes the substitution T303L, Y360F and G402A compared to SEQ ID NO:2). Suitably, the modified PPO may have an amino acid sequence having at least 50% sequence identity to SEQ ID NO: 143. Suitably, the modified PPO may have an amino acid sequence having at least 85%, 90%, 91%, 92%, 93%, 94%, 95%, 96%, 97%, 98%, 99% or 100% sequence identity to SEQ ID NO: 143. Suitably, the modified PPO may comprise an amino acid sequence according to SEQ ID NO: 143. Suitably, the modified PPO may essentially consist of an amino acid sequence according to SEQ ID NO: 143.

Suitably, the modified PPO may have an amino acid sequence having at least 30% sequence identity to SEQ ID NO: 144 (wherein the amino acid sequence includes the substitution T303L, Y359T, V363L, T429R and L477M compared to SEQ ID NO:2).

Suitably, the modified PPO may have an amino acid sequence having at least 50% sequence identity to SEQ ID NO: 144. Suitably, the modified PPO may have an amino acid sequence having at least 85%, 90%, 91%, 92%, 93%, 94%, 95%, 96%, 97%, 98%, 99% or 100% sequence identity to SEQ ID NO: 144. Suitably, the modified PPO may comprise an amino acid sequence according to SEQ ID NO: 144. Suitably, the modified PPO may essentially consist of an amino acid sequence according to SEQ ID NO: 144.

Suitably, the modified PPO may have an amino acid sequence having at least 30% sequence identity to SEQ ID NO: 145 (wherein the amino acid sequence includes the substitution T303L, Y424V and L477M compared to SEQ ID NO:2). Suitably, the modified PPO may have an amino acid sequence having at least 50% sequence identity to SEQ ID NO: 145. Suitably, the modified PPO may have an amino acid sequence having at least 85%, 90%, 91%, 92%, 93%, 94%, 95%, 96%, 97%, 98%, 99% or 100% sequence identity to SEQ ID NO: 145. Suitably, the modified PPO may comprise an amino acid sequence according to SEQ ID NO: 145. Suitably, the modified PPO may essentially consist of an amino acid sequence according to SEQ ID NO: 145.

Suitably, the modified PPO may have an amino acid sequence having at least 30% sequence identity to SEQ ID NO: 146 (wherein the amino acid sequence includes the substitution Y424M V459Q and L477M compared to SEQ ID NO:2). Suitably, the modified PPO may have an amino acid sequence having at least 50% sequence identity to SEQ ID NO: 146. Suitably, the modified PPO may have an amino acid sequence having at least 85%, 90%, 91%, 92%, 93%, 94%, 95%, 96%, 97%, 98%, 99% or 100% sequence identity to SEQ ID NO: 146. Suitably, the modified PPO may comprise an amino acid sequence according to SEQ ID NO: 146. Suitably, the modified PPO may essentially consist of an amino acid sequence according to SEQ ID NO: 146.

Suitably, the modified PPO may have an amino acid sequence having at least 30% sequence identity to SEQ ID NO: 147 (wherein the amino acid sequence includes the substitution T303L, Y359D and V363M compared to SEQ ID NO:2). Suitably, the modified PPO may have an amino acid sequence having at least 50% sequence identity to SEQ ID NO: 147. Suitably, the modified PPO may have an amino acid sequence having at least 85%, 90%, 91%, 92%, 93%, 94%, 95%, 96%, 97%, 98%, 99% or 100% sequence identity to SEQ ID NO: 147. Suitably, the modified PPO may comprise an amino acid sequence according to SEQ ID NO: 147. Suitably, the modified PPO may essentially consist of an amino acid sequence according to SEQ ID NO: 147.

Suitably, the modified PPO may have an amino acid sequence having at least 30% sequence identity to SEQ ID NO: 148 (wherein the amino acid sequence includes the substitution T303L, Y359C, Y360F, Y424M and L477M compared to SEQ ID NO:2).

Suitably, the modified PPO may have an amino acid sequence having at least 50% sequence identity to SEQ ID NO: 148. Suitably, the modified PPO may have an amino acid sequence having at least 85%, 90%, 91%, 92%, 93%, 94%, 95%, 96%, 97%, 98%, 99% or 100% sequence identity to SEQ ID NO: 148. Suitably, the modified PPO may comprise an amino acid sequence according to SEQ ID NO: 148. Suitably, the modified PPO may essentially consist of an amino acid sequence according to SEQ ID NO: 148.

Suitably, the modified PPO may have an amino acid sequence having at least 30% sequence identity to SEQ ID NO: 149 (wherein the amino acid sequence includes the substitution Y359D and V363L compared to SEQ ID NO:2). Suitably, the modified PPO may have an amino acid sequence having at least 50% sequence identity to SEQ ID NO: 149.

Suitably, the modified PPO may have an amino acid sequence having at least 85%, 90%, 91%, 92%, 93%, 94%, 95%, 96%, 97%, 98%, 99% or 100% sequence identity to SEQ ID NO: 149. Suitably, the modified PPO may comprise an amino acid sequence according to SEQ ID NO: 149. Suitably, the modified PPO may essentially consist of an amino acid sequence according to SEQ ID NO: 149.

Suitably, the modified PPO may have an amino acid sequence having at least 30% sequence identity to SEQ ID NO: 150 (wherein the amino acid sequence includes the substitution V363L, G402A and L477N compared to SEQ ID NO:2). Suitably, the modified PPO may have an amino acid sequence having at least 50% sequence identity to SEQ ID NO: 150. Suitably, the modified PPO may have an amino acid sequence having at least 85%, 90%, 91%, 92%, 93%, 94%, 95%, 96%, 97%, 98%, 99% or 100% sequence identity to SEQ ID NO: 150. Suitably, the modified PPO may comprise an amino acid sequence according to SEQ ID NO: 150. Suitably, the modified PPO may essentially consist of an amino acid sequence according to SEQ ID NO: 150.

Suitably, the modified PPO may have an amino acid sequence having at least 30% sequence identity to SEQ ID NO: 151 (wherein the amino acid sequence includes the substitution T303L, G402A and L477N compared to SEQ ID NO:2). Suitably, the modified PPO may have an amino acid sequence having at least 50% sequence identity to SEQ ID NO: 151. Suitably, the modified PPO may have an amino acid sequence having at least 85%, 90%, 91%, 92%, 93%, 94%, 95%, 96%, 97%, 98%, 99% or 100% sequence identity to SEQ ID NO: 151. Suitably, the modified PPO may comprise an amino acid sequence according to SEQ ID NO: 151. Suitably, the modified PPO may essentially consist of an amino acid sequence according to SEQ ID NO: 151.

In any aspect of the invention as described herein, a PPO enzyme which has resistance to a compound which inhibits PPO enzymatic activity, whether partial or complete, may comprise an amino acid which has at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to any of SEQ ID NOs 1 or 2 or any modified PPO disclosed herein, which may also be referred to as reference sequences.

In any aspect of the invention, a modified PPO enzyme which has resistance to a compound which inhibits PPO enzymatic activity may be a homologue of any of SEQ ID NOs: 1, 2, 4-151, 153-302, or 305-336. As used herein, “homologue” refers to a protein that is functionally equivalent, i.e. has the same enzymatic activity as an enzyme having an amino acid sequence according to SEQ ID NO 1, 2, 4-151, 153-302, or 305-336 (i.e. acts as a PPO enzyme as defined herein), but may have a limited number of amino acid substitutions, deletions, insertions or additions in the amino acid sequence. Homologues need not display the same level of enzymatic activity. Homologues may have lower sequences identities, for example, at least 20%, at least 25%, at least 30%, at least 35% or at least 40% or more sequence identity to a PPO enzyme identified herein, but are capable of carrying out the same enzymatic reaction. The invention, therefore, includes any isoforms of PPO enzymes and their mutations as defined herein.

“Identity” or “percent identity” refers to the degree of sequence variation between two given nucleic acid or amino acid sequences. For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters. Optimal alignment of sequences for comparison can be conducted, e.g., by the local homology algorithm of Smith & Waterman, Adv. Appl. Math.2: 482 (1981), by the homology alignment algorithm of Needleman & Wunsch, J. Mol. Biol.48: 443 (1970), by the search for similarity method of Pearson & Lipman, Proc. Nat'l. Acad. Sci. USA 85: 2444 (1988), by computerized implementations of these algorithms (GAP, BESTFIT, FASTA, and TFASTA in the Wisconsin Genetics Software Package, Genetics Computer Group, 575 Science Dr., Madison, WI), or by visual inspection. One example of an algorithm that is suitable for determining percent sequence identity and sequence similarity is the BLAST algorithm, which is described in Altschul et al., J. Mol. Biol.215: 403-410 (1990). Software for performing BLAST analyses is publicly available through the National Center for Biotechnology Information (on the world wide web at ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighbourhood word score threshold (Altschul et al., J. Mol. Biol.215: 403-410 (1990)). These initial neighbourhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see, Henikoff & Henikoff, Proc. Natl. Acad. Sci. USA 89: 10915 (1989)). In addition to calculating percent sequence identity, the BLAST algorithm also performs a statistical analysis of the similarity between two sequences (see, e.g., Karlin & Altschul, Proc. Nat'l. Acad. Sci. USA 90: 5873-5787 (1993)). One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is less than about 0.1. In one embodiment less than about 0.01, and in one embodiment less than about 0.001.

Unless otherwise stated, “percent identity” as used herein refers to the value obtained using the Needleman and Wunsch algorithm ((1970) J. Mol. Biol. 48:443-453) implemented in the EMBOSS Needle alignment tool using default matrix files EBLOSUM62 for protein with default parameters (Gap Open=10, Gap Extend=0.5, End Gap Penalty=False, End Gap Open=10, End Gap Extend=0.5) or DNAfull for nucleic acids with default parameters (Gap Open=10, Gap Extend=0.5, End Gap Penalty=False, End Gap Open=10, End Gap Extend=0.5); or any equivalent program thereof. EMBOSS Needle is available, e.g., from EMBL-EBI such as at the following website: ebi.ac.uk/Tools/psa/emboss_needle/ and as described in the following publication: “The EMBL-EBI search and sequence analysis tools APIs in 2019.” Madeira et al. Nucleic Acids Research, June 2019, 47(W1):W636-W641. The term “equivalent program” as used herein refers to any sequence comparison program that, for any two sequences in question, generates an alignment having identical nucleotide or amino acid residue matches and an identical percent sequence identity when compared to the corresponding alignment generated by EMBOSS Needle

A PPO enzyme encoded by a nucleic acid or a PPO enzyme of the invention may be a functional fragment of a PPO enzyme as described herein. A “functional fragment” refers to a protein fragment that retains protein function. As such, a functional fragment of a PPO enzyme is a fragment, portion or part of a PPO protein that is still capable of catalysing the 6-electron oxidation of protoporphyrinogen-IX to form protoporphyrin-IX.

The mutations defined herein may be located at a position ‘corresponding to’ an amino acid position listed in another PPO enzyme. It is possible to compare PPO polypeptides by sequence comparison and locating conserved regions that correspond to the amino acid positions listed. The term “equivalent amino acids” or “corresponding amino acids” refers to amino acids in a sequence of interest, which correspond to those amino acids of an identified reference sequence, typically herein the reference sequence is SEQ ID NO:1 or 2 for PPO enzymes. A region of equivalent or corresponding amino acids may be determined by aligning the amino acid sequences of the proteins from the different species, using an alignment program such as BLAST® or ClustalW. Note that the corresponding positions in a sequence of interest should be determined by comparison with a like for like reference sequence. Should it be desired to determine the corresponding positions in a PPO enzyme lacking a targeting peptide, then the reference PPO sequence should also lack a targeting peptide. Suitably in such embodiments, the reference sequence used herein may be SEQ ID NO: 1, 2 (with transit peptides), 153 or 154 (without transit peptides). Any amino acid positions listed herein in relation to a PPO enzyme sequence comprising a targeting peptide still apply to a PPO enzyme sequence from the same organism without a targeting peptide.

A “corresponding” amino acid position to a given SEQ ID NO is determined using Geneious as a global alignment with free end gaps having the following parameters: cost matrix Blossum 62, gap open penalty 12, gap extension penalty 3, refinement iterations 2; or an equivalent program thereof.

Unless otherwise stated, a “corresponding” amino acid position to a given SEQ ID NO is determined using EMBOSS Needle default parameters: BLOSUM62; Gap Open 10, GAP EXTEND 0.5; END GAP OPEN 10 and END GAP EXTEND 0.5, or an equivalent program thereof. See, Madeira et al. (2022) Nucleic Acids Research, 1 Jul. 2022, 50(W1):W276-W279; PMID: 35412617 PMCID: PMC9252731. The term “equivalent program” as used herein refers to any sequence comparison program that, for any two sequences in question, generates an alignment having identical corresponding nucleotide or amino acid residue matches when compared to the corresponding alignment generated by the program provided above.

Mutations may include deletions or substitutions or combinations thereof. For example, the mutations may be conservative or non-conservative amino acid substitutions.

“Conservative amino acid substitutions” refer to the interchangeability of residues having similar side chains, and thus typically involves substitution of an amino acid in a polypeptide with amino acids within the same or similar defined class of amino acids. By way of example, an amino acid with an aliphatic side chain may be substituted with another aliphatic amino acid, e.g., alanine, valine, leucine, and isoleucine; an amino acid with a hydroxyl side chain may be substituted with another amino acid with a hydroxyl side chain, e.g., serine and threonine; amino acids having aromatic side chains may be substituted with another amino acid having an aromatic side chain, e.g., phenylalanine, tyrosine, tryptophan, and histidine; an amino acid with a basic side chain may be substituted with another amino acid with a basic side chain, e.g., lysine and arginine; an amino acid with an acidic side chain may be substituted with another amino acid with an acidic side chain, e.g., aspartic acid or glutamic acid; and a hydrophobic or hydrophilic amino acid may be substituted with another hydrophobic or hydrophilic amino acid, respectively. Exemplary conservative substitutions are provided below:

| Residue | Possible Conservative Substitutions |
|---|---|
| A, L, V, I | Other aliphatic (A, L, V, I) |
| | Other non-polar (A, L, V, I, G, M) |
| G, M | Other non-polar (A, L, V, I, G, M) |
| D, E | Other acidic (D, E) |
| K, R | Other basic (K, R) |
| N, Q, S, T | Other polar |
| H, Y, W, F | Other aromatic (H, Y, W, F) |
| C | None |
| P | None |

“Non-conservative substitution” refers to substitution of an amino acid in a polypeptide with an amino acid with significantly differing side chain properties. Non-conservative substitutions may use amino acids between, rather than within, the defined groups and may affect (a) the structure of the peptide backbone in the area of the substitution (e.g., proline for Glycine) (b) the charge or hydrophobicity, or (c) the bulk of the side chain. By way of example, an exemplary non-conservative substitution can be an acidic amino acid substituted with a basic or aliphatic amino acid; an aromatic amino acid substituted with a small amino acid; and a hydrophilic amino acid substituted with a hydrophobic amino acid.

“Deletion” refers to modification of a polypeptide by removal of one or more amino acids in comparison to a wild-type or control polypeptide. Deletions can comprise removal of 1 or more amino acids, 2 or more amino acids, or 3 or more amino acids of the polypeptide while retaining enzymatic activity. Deletions can comprise a continuous segment or can be discontinuous.

Suitably the modified PPO enzyme may further comprise a transit peptide, suitably a mitochondrial and/or chloroplast transit peptide, suitably at the N or C terminus thereof.

Suitably, at the N-terminus thereof. In one embodiment, the transit peptide is a mitochondrial transit peptide. In one embodiment, the transit peptide is a chloroplast transit peptide. In one embodiment, the transit peptide comprises a chloroplast transit peptide and a mitochondrial transit peptide.

Suitably such mitochondrial or chloroplast transit peptides are present in wild type PPO enzymes. Suitably therefore, the modified PPO enzyme may comprise a native, endogenous mitochondrial or chloroplast transit peptide. Alternatively, the endogenous mitochondrial or chloroplast transit peptide may be replaced with a heterologous mitochondrial or chloroplast transit peptide, derived from an enzyme or a different PPO enzyme.

Suitably therefore, in some embodiments, the modified PPO enzyme may further be modified by the addition of a sequence. Suitably by the addition of a sequence to its N or C terminus. Suitably the sequence is a heterologous transit peptide. Suitably the heterologous transit peptide is added to the C terminus of the PPO enzyme. Suitably the heterologous transit peptide is added to the N terminus of the PPO enzyme

In one embodiment, therefore, the modified PPO enzyme comprises a heterologous transit peptide, suitably a heterologous mitochondrial or chloroplast transit peptide, suitably at the N terminus thereof. In one embodiment, the transit peptide is a heterologous mitochondrial transit peptide. In one embodiment, the transit peptide is a heterologous chloroplast transit peptide. In one embodiment, the transit peptide is a heterologous dual targeting chloroplast/mitochondrial transit peptide. Suitably the heterologous transit peptide is added to the modified PPO enzyme, suitably such that the modified PPO enzyme is produced as a fusion protein with the heterologous transit peptide.

Suitably the heterologous mitochondrial or chloroplast transit peptide is a plant mitochondrial or chloroplast transit peptide. Suitably the heterologous mitochondrial or chloroplast transit peptide may be derived from a heterologous PPO enzyme, suitably from a wild type heterologous PPO enzyme. Suitably the heterologous mitochondrial or chloroplast transit peptide may be derived from a heterologous plant PPO enzyme. Non-limiting examples of transit peptides include those set forth in WO2017198859, U.S. Ser. No. 10/745,712 and U.S. Ser. No. 10/563,220, each of which in incorporated by reference in their entirety.

Suitable transit peptides may be selected from:
MELSLLRPTTQSLLPSFSKPNLRLNVYKPLRLRC (SEQ ID NO: 303); or
MVAAAMATAPSAGVPPLRGTRGPARFRIRGVSVRC (SEQ ID NO: 304).

For example, the modified PPO enzyme may comprise a sequence according to any one of SEQ ID NOs: 153 to 302 or 321 to 336. Suitably, the modified PPO enzyme may comprise a sequence having at least 30% sequence identity to a sequence according to any one of SEQ ID NOs: 153 to 302 or 321 to 336. For example, at least 30%, at least 35%, at least 40%, at least 45%, at least 50%, at least 55%, at least 60%, at least 70%, at least 75%, at least 80%, at least 85%, at least 90%, at least 91%, at least 92%, at least 93%, at least 94%, at least 95%, at least 98% or at least 99%. Suitably, such a modified PPO enzyme comprising a sequence according to any one of SEQ ID NOs: 153 to 302 or 321 to 336 may further comprise a transit peptide at the C or N terminus thereof, suitably which may be added thereto. Suitably which may be a mitochondrial transit peptide and/or a chloroplastic transit peptide. Suitably, in either case, which may be a heterologous transit peptide.

Suitably, the modified PPO enzyme may comprise both a mitochondrial transit peptide and chloroplastic transit peptide. For example, a chloroplastic and mitochondrial transit peptide as described herein.

It will be understood that when reference is made to specific amino acid positions with reference to a sequence including a transit peptide, these positions will differ by the number of amino acids which comprise a transit peptide in a modified PPO enzyme that does not include a transit peptide. Any references herein to sequences including a transit peptide apply equally to the corresponding sequence provided herein without a transit peptide under SEQ ID NOs 153-302 or 321-336.

For example, positions 362, 365, and 479 of SEQ ID NO: 1 (wild type Arabidopsis thaliana PPO including naturally occurring transit peptide) would respectively be denoted as positions 328, 331, and 445 of SEQ ID NO: 153 (wild type Arabidopsis thaliana PPO without transit peptide).

For example, positions 360, 363, and 477 of SEQ ID NO: 2 (wild type Setaria Italica PPO including naturally occurring transit peptide) would respectively be denoted as positions 325, 328 and 442 of SEQ ID NO: 154 (wild type Setaria Italica PPO without transit peptide).

In some examples, the modified PPO enzyme may comprise one or more additional elements, regions or domains that act to improve or help purification, expression, and/or stability of the modified PPO enzyme. For example, the modified PPO enzymes provided herein may include one or more purification tags, detections tags and/or cleavage sites.

A detection tag may be amino acid sequence that can be bound by a specific binding partner or emits a signal to allow the modified PPO enzyme to be detected or visualised. Examples of detection tags include V5 tag, Xpress tag, myc tag, 6×His, GST, BioEase tag, capTEV tag, florescent tags (such as GFP), Lumio tag, HA tag, and FLAG tag.

A purification tag may be an amino acid sequence that can be used to help purify the modified PPO enzymes when produced by recombinant methods. For example, purification tags may allow for binding of the modified PPO enzyme to a resin or bead via the purification tag. Examples of purification tags include, 6×His, GST, BioEase tag, capTEV tag. For example, the modified PPO enzymes provided herein may include a 6×His tag (i.e. a tag including 6 histidine residues). Suitably, the modified PPO enzymes provided herein may include a 6×His tag according to SEQ ID NO: 375.

A cleavage sit refers to an amino acid sequence that may be cleaved or cut by a specific cleavage enzyme such as a protease. Cleavage sites may allow for simplified removal of tags that may be included. Cleavage may be carried our after purification and before transfer of a recombinant protein into a host. For example, a cleavage site may be placed adjacent to a tag allowing cleavage of the tag using a protease enzyme. Examples of cleavage sites include Tobacco Etch Virus (TEV) site, enterokinase (EK) cleavage site, HRV3C site, and Factor Xa site. Suitably, the modified PPO enzymes provided herein may include a TEV site. For example as TEV site according to SEQ ID NO: 376. In some example, the modified PPO enzymes provided herein include a His-Tag and TEV site as defined in SEQ ID NO: 377 or 378.

Combined Resistance and/or Additional Traits of Interest

The plants or parts thereof of the invention may further be modified to comprise an additional trait of interest. In one embodiment, the additional trait of interest increases resistance to a different compound which inhibits a different plant metabolic process. Suitably, the plants or parts thereof of the invention may further be modified to comprise increased resistance to a compound which inhibits a plant metabolic process other than the those involving a PPO enzyme. Suitably this may be regarded as ‘stacking’ of resistance. Suitably resistance to a compound which inhibits PPO enzymes may be stacked with resistance to another compound which inhibits a different enzyme or metabolic pathway, in the plants of the invention. Suitably therefore, the plant or part thereof exhibits a second compound-resistant trait.

Suitably the plants or parts thereof of the invention may further be modified to comprise increased resistance to a compound which inhibits a different plant enzyme to PPO. Suitably the plants or parts thereof of the invention may further be modified to comprise increased resistance to a compound which inhibits an essential plant metabolic process. By inhibiting a plant metabolic process, this may mean inhibiting one or more enzymes of a plant metabolic process. Suitably the plants or parts thereof of the invention may further be modified to comprise increased resistance to a compound which is not a compound that targets PPO enzymes, but which targets a different essential plant enzyme and metabolic process.

Suitably the plant or part thereof may comprise an additional modified enzyme, suitably which has been modified to increase its resistance to the compound, which inhibits a different plant enzyme to PPO. Suitably such compounds are herbicides. Suitably the plant or part thereof may comprise increased resistance to a herbicide which inhibits PPO enzymatic activity and increased resistance to another herbicide, suitably which inhibits a different plant metabolic process. Suitably therefore the plant or part thereof exhibits a second herbicide-resistant trait.

The modified PPO enzymes and variants thereof provided herein can therefore be stacked with one or more additional modified enzymes which confers a desirable trait such as, for example, insect, disease or herbicide resistance or other desirable agronomic traits of interest including, but not limited to, traits associated with high oil content; traits associated with increase protein content, increased digestibility; balanced amino acid content; improved drought resistance, modified maturity and/or flowering time, and high energy content. Such traits may refer to properties of both seed and non-seed plant tissues, or to food or feed prepared from plants or seeds having such traits.

As used herein, gene or trait “stacking” comprises combining desired genes or traits into one transgenic plant line. Stacking can include the introduction of transgenic traits of interest, genome edited traits of interest or native traits of interest. The additional polynucleotide can be introduced by a variety of approaches, as described herein in relation to polynucleotides encoding the modified PPO enzymes, including by transgenic means, by breeding, targeted integration via genome editing system, or by genome editing. As one approach, plant breeders stack transgenic traits by making crosses between parents that each have a desired trait and then identifying offspring that have both of these desired traits (so-called “breeding stacks”). Another way to stack genes is by transferring two or more genes into the cell nucleus of a plant at the same time during transformation. In embodiments, the two or more genes may be transferred via distinct expression cassettes or via a common expression cassette. Another way to stack genes is by re-transforming a transgenic plant comprising a desired trait with another gene of interest conferring another desired trait to thereby provide a progeny transgenic plant comprising the combination of traits. Such methods can include, for example, random integration techniques or targeted integration via a gene editing system such as Crispr or meganucleases. For example, gene stacking can be used to combine two different insect resistance traits, two different herbicide resistance traits, two different agronomic performance traits, an insect resistance trait with a disease resistance trait, a herbicide resistance trait (such as, for example, Bt11), or an agronomic performance trait, etc. The use of a selectable marker in addition to a gene of interest would also be considered gene stacking. In embodiments, the offspring or progeny plant having the desired combination of traits is identified through the use of genetic markers or molecular markers including but not limited to SNPs, QTLs, primers or probes directed to desired trait-associated genes or transgenes, promoters, microRNAs, siRNAs, mRNAs, ds RNAs, transcriptional profiles, and methylation patterns.

In some embodiments, a polynucleotide or vector described herein, can include an additional coding sequence for one or more polypeptides or double stranded RNA molecules (dsRNA) of interest for agronomic traits that primarily are of benefit to a seed company, grower or grain processor. A polypeptide of interest can be any polypeptide encoded by a nucleotide sequence of interest, such as an enzyme. Non-limiting examples of polypeptides of interest that are suitable for production in plants include those resulting in agronomically important traits such as herbicide resistance (also sometimes referred to as “herbicide tolerance”), disease resistance, virus resistance, bacterial pathogen resistance, insect resistance, nematode resistance, or fungal resistance, such as modified enzymes conferring these traits. See, e.g., U.S. Pat. Nos. 5,569,823; 5,304,730; 5,495,071; 6,329,504; and 6,337,431. The polypeptide also can be one that increases plant vigor or yield (including traits that allow a plant to grow at different temperatures, soil conditions and levels of sunlight and precipitation), or one that allows identification of a plant exhibiting a trait of interest (e.g., a selectable marker, seed coat color, relative maturity group, etc.). Various polypeptides of interest, as well as methods for introducing these polypeptides into a plant, are described, for example, in U.S. Pat. Nos. 4,761,373; 4,769,061; 4,810,648; 4,940,835; 4,975,374; 5,013,659; 5,162,602; 5,276,268; 5,304,730; 5,495,071; 5,554,798; 5,561,236; 5,569,823; 5,767,366; 5,879,903, 5,928,937; 6,084,155; 6,329,504 and 6,337,431; as well as US Patent Publication No. 2001/0016956.

Suitably the additional polypeptide of interest to be stacked with the modified PPO enzyme provided herein may comprise any known polypeptide of interest or a modified enzyme in the art that has been modified to increase resistance to a known compound which inhibits a plant metabolic process. Suitably such compounds are herbicides. Suitably therefore the additional polypeptide of interest or modified enzyme may provide additional herbicide resistance trait, suitably by providing increased herbicide resistance. Suitably the plant or part thereof may comprise a modified PPO enzyme which provides increased resistance to a herbicide which inhibits PPO and an additional enzyme which provides increased resistance to another herbicide, suitably which inhibits a different plant metabolic process.

In some embodiments, the polypeptides of interest stacked with the modified PPO enzymes provide herein confer resistance/tolerance to a herbicide that inhibits the growing point or meristem, such as an imidazalinone or a sulfonylurea may be suitable in some embodiments. Exemplary polynucleotides in this category code for mutant ALS and AHAS enzymes as described, e.g., in U.S. Pat. Nos. 5,767,366 and 5,928,937. U.S. Pat. Nos. 4,761,373 and 5,013,659 are directed to plants resistant to various imidazalinone or sulfonamide herbicides. U.S. Pat. No. 4,975,374 relates to plant cells and plants containing a nucleic acid encoding a modified glutamine synthetase (GS) enzyme resistant to inhibition by herbicides that are known to inhibit GS, e.g., phosphinothricin and methionine sulfoximine. U.S. Pat. No. 5,162,602 discloses plants resistant to inhibition by cyclohexanedione and aryloxyphenoxypropanoic acid herbicides. The resistance is conferred by a modified acetyl coenzyme A carboxylase (ACCase) enzyme. Additional herbicide tolerant traits that confer tolerance to ACCase inhibitors can be found in WO2014144951, WO2017138986, WO2018205995, WO2021088601, WO2011028836, and WO2011028833.

In some embodiments, the polypeptide of interest stacked with the modified PPO enzymes provided herein confer resistance to glyphosate are also suitable for the disclosure. See, e.g., U.S. Pat. Nos. 4,940,835 and 4,769,061. U.S. Pat. No. 5,554,798 discloses transgenic glyphosate resistant maize plants, which resistance is conferred by a modified 5-enolpyruvyl-3-phosphoshikimate (EPSP) synthase gene. Polynucleotides coding for resistance to phosphono compounds such as glufosinate ammonium or phosphinothricin, and pyridinoxy or phenoxy propionic acids and cyclohexones are also suitable. See, European Patent Application No. 0 242 246. See also, U.S. Pat. Nos. 5,879,903, 5,276,268 and 5,561,236.

Additional herbicide tolerant traits that can be stacked with the modified PPOs disclosed herein include, PPO tolerant traits including, for example, one or more PPO trait set forth in US20190062777, U.S. Ser. No. 10/370,677, U.S. Ser. No. 11/124,803, WO2017217793, WO2020251313, U.S. Ser. No. 10/392,630, U.S. Ser. No. 10/378,023, WO2016099153, WO2019117579, WO2019117578, and U.S. Ser. No. 10/100,329, each of which is herein incorporated by reference in their entirety. HPPD tolerant traits include: WO2009144079, U.S. Pat. No. 8,642,748, EP2453012, WO2013026740, U.S. Pat. No. 9,078,446, U.S. Ser. No. 10/793,872, U.S. Ser. No. 10/508,089, U.S. Ser. No. 10/400,249, U.S. Ser. No. 10/597,674, WO2018119364, WO2018119361, U.S. Ser. No. 11/180,770, US20200157086, US20210147866, U.S. Ser. No. 11/279,944, US202000331866, WO2019227036, WO2019227028, WO2022115296, WO2011068567, and those disclosed in Maeda et al. (2019) Science, vol 365, issue 6451, pp 393-396, each of which is herein incorporated by reference in their entirety. ACCase tolerant traits include: US20120284812, US20120284853, US20160108423, US20160244780, US20160264990, US20170275645, US20210153448, U.S. Ser. No. 10/696,975B2, U.S. Ser. No. 10/370,678, CN109082416, U.S. Ser. No. 10/694,694, US20170265469, US20170231225, each of which is herein incorporated by reference. Dicamba tolerant traits include, for example, RE45048 or U.S. Pat. No. 7,884,262. Various traits the confer tolerance to AOPP herbicides, phenoxy acid herbicides and/or pyridinyloxy acid herbicides include, for example, U.S. Ser. No. 10/174,337, U.S. Pat. No. 8,278,505, WO05107437, WO11022469, U.S. Ser. No. 10/023,874, and US2019241903 (and other traits therein), each of which is herein incorporated by reference. Additional herbicides tolerant traits of interest for stacking include glucosyl transferase polypeptides as set forth in 2018213022 or Solanesyl Diphosphate Synthase polypeptides as set forth in WO2020236790, or a BIO3-BIO1 and/or BioA enzyme as described in European patent application EP23154964.3, each of which is herein incorporated by reference in their entirety.

Other suitable polynucleotides include those which confer resistance to herbicides that inhibit photosynthesis, such as a triazine and a benzonitrile (nitrilase) See, U.S. Pat. No. 4,810,648. Additional suitable polynucleotides coding for herbicide resistance include those coding for resistance to 2,2-dichloropropionic acid, sethoxydim, haloxyfop, imidazolinone herbicides, sulfonylurea herbicides, triazolopyrimidine herbicides, s-triazine herbicides and bromoxynil. Also suitable are polynucleotides encoding modified enzymes that confer resistance to a protox enzyme, or that provide enhanced resistance to plant diseases; enhanced tolerance of adverse environmental conditions (abiotic stresses) including but not limited to drought, excessive cold, excessive heat, or excessive soil salinity or extreme acidity or alkalinity; and alterations in plant architecture or development, including changes in developmental timing. See, e.g., U.S. Patent Publication No. 2001/0016956 and U.S. Pat. No. 6,084,155.

Disease resistance proteins such as enzymes that increase resistance to various plant disease including rust, include, but are not limited to, one or more of the various resistance genes set forth in: WO2019103918; WO202100878; WO2021022022; WO2021260673; WO2022173659; WO2022159341; WO2021154632A1, WO2021022026, WO2021022101, US20220135997; U.S. Ser. No. 10/842,097; or WO2022140257; each of which is incorporated by reference in their entirety. In other embodiments, the modified PPO enzymes described herein or active variant or fragments thereof is stacked with a native trait that confers disease resistance. For example, the various intervals, locus or resistance genes as set forth in WO2009079729, U.S. Pat. No. 9,091,681, WO2010009404, WO2017222827, WO2021000878, WO2021022026, WO2021022101, WO2021154632, WO2022173659, (each of which is incorporated by reference in their entirety) can used to introduce a trait of interest. Disease resistance proteins and/or native traits that increase resistance to various plant diseases including NCLB include, for example, U.S. Pat. No. 8,921,646, US2021000059, U.S. Ser. No. 10/858,668, US20200199610, WO2022/013268, WO2022/013268, U.S. Pat. No. 9,040,772, U.S. Ser. No. 10/897,862, EP3839073, each of which is herein incorporated by reference.

Additional suitable polynucleotides include those coding for insecticidal polypeptides such as insecticidal enzymes. These polypeptides may be produced in amounts sufficient to control, for example, insect pests (i.e., insect controlling amounts). It is recognized that the amount of production of an insecticidal polypeptide in a plant necessary to control insects or other pests may vary depending upon the cultivar, type of pest, environmental factors and the like. Polynucleotides useful for additional insect or pest resistance include, for example, those that encode toxins identified in Bacillus organisms. Polynucleotides comprising nucleotide sequences encoding Bacillus thuringiensis (Bt) Cry proteins from several subspecies have been cloned and recombinant clones have been found to be toxic to lepidopteran, dipteran and/or coleopteran insect larvae. Examples of such Bt insecticidal proteins include the Cry proteins such as Cry1Aa, Cry1Ab, Cry1Ac, Cry1B, Cry1C, Cry1D, Cry1Ea, Cry1Fa, Cry3A, Cry9A, Cry9B, Cry9C, and the like, as well as vegetative insecticidal proteins such as Vip1, Vip2, Vip3, and the like. A full list of Bt-derived proteins can be found on the worldwide web at Bacillus thuringiensis Toxin Nomenclature Database maintained by the University of Sussex (see also, Crickmore et al. (1998) Microbiol. Mol. Biol. Rev. 62:807-813).

In embodiments, an additional polypeptide is an insecticidal polypeptide, such as an enzyme, derived from a non-Bt source, including without limitation, an alpha-amylase, a peroxidase, a cholesterol oxidase, a patatin, a protease, a protease inhibitor, a urease, an alpha-amylase inhibitor, a pore-forming protein, a chitinase, a lectin, an engineered antibody or antibody fragment, a Bacillus cereus insecticidal protein, a Xenorhabdus spp. (such as X. nematophila or X. bovienii) insecticidal protein, a Photorhabdus spp. (such as P. luminescens or P. asymobiotica) insecticidal protein, a Brevibacillus spp. (such as B. laterosporous) insecticidal protein, a Lysinibacillus spp. (such as L. sphearicus) insecticidal protein, a Chromobacterium spp. (such as C. subtsugae or C. piscinae) insecticidal protein, a Yersinia spp. (such as Y. entomophaga) insecticidal protein, a Paenibacillus spp. (such as P. propylaea) insecticidal protein, a Clostridium spp. (such as C. bifermentans) insecticidal protein, a Pseudomonas spp. (such as P. fluorescens) and a lignin.

In certain embodiments, the additional polypeptide or enzyme is a resistance protein such as an enzyme conferring enhanced pathogen resistance, such as enhanced resistance to any one of the following pathogens: soy cyst nematode, bacterial pustule, root knot nematode, frog eye leaf spot, phytopthora, brown stem rot, nematode, Asian Soybean Rust, smut, Golovinomyces cichoracearum, Erysiphe cichoracearum, Blumeria graminis, Podosphaera xanthii, Sphaerotheca fuliginea, Pythium ultimum, Uncinula necator, Mycosphaerella pinodes, Magnaporthe grisea, Bipolaris oryzae, Magnaporthe grisea, Rhizoctonia solani, Phytophthora sojae, Schizaphis graminum, Bemisia tabaci, Rhopalosiphum maidis, Deroceras reticulatum, Diatraea saccharalis, Schizaphis graminum, Myzus persicae, Sclerotinia sclerotiorum, Macrophomina phaseolina, or Fusarium virguliforme. Exemplary polynucleotides encoding proteins that confer increased pathogen resistance that may be stacked with the modified PPO enzymes of the invention include polynucleotides encoding proteins such as enzymes that confer increased ASR resistance as described in US Patent publication Nos. US 20200354739 and PCT Publications Nos. WO2019103918, WO2021154632A1, WO2021022022, WO2021022026, WO2021022101, WO2021260673, and WO2021263249, each of which is incorporated by reference in its entirety.

Polypeptides that are suitable for production in plants further include those that improve or otherwise facilitate the conversion of harvested plants or plant parts into a commercially useful product, including, for example, increased or altered carbohydrate content or distribution, improved fermentation properties, increased oil content, increased protein content, improved digestibility, and increased nutraceutical content, e.g., increased phytosterol content, increased tocopherol content, increased stanol content or increased vitamin content. Polypeptides of interest also include, for example, those resulting in or contributing to a reduced content of an unwanted component in a harvested crop, e.g., phytic acid, or sugar degrading enzymes. By “resulting in” or “contributing to” is intended that the polypeptide of interest can directly or indirectly contribute to the existence of a trait of interest (e.g., increasing cellulose degradation by the use of a heterologous cellulase enzyme). Any such polypeptides may be stacked with the modified PPO enzymes of the invention.

In some embodiments, the polypeptide contributes to improved digestibility for food or feed. Xylanases are hemicellulolytic enzymes that improve the breakdown of plant cell walls, which leads to better utilization of the plant nutrients by an animal. This leads to improved growth rate and feed conversion. Also, the viscosity of the feeds containing xylan can be reduced. Heterologous production of xylanases in plant cells also can facilitate lignocellulosic conversion to fermentable sugars in industrial processing. Numerous xylanases from fungal and bacterial microorganisms have been identified and characterized (see, e.g., U.S. Pat. No. 5,437,992; Coughlin et al. (1993) “Proceedings of the Second TRICEL Symposium on Trichoderma reesei Cellulases and Other Hydrolases” Espoo; Souminen and Reinikainen, eds. (1993) Foundation for Biotechnical and Industrial Fermentation Research 8:125-135; U.S. Patent Publication No. 2005/0208178; and PCT Publication No. WO 03/16654). In particular, three specific xylanases (XYL-I, XYL-II, and XYL-III) have been identified in T. reesei (Tenkanen et al. (1992) Enzyme Microb. Technol. 14:566; Torronen et al. (1992) Bio/Technology 10:1461; and Xu et al. (1998) Appl. Microbiol. Biotechnol. 49:718). Any such polypeptides may be stacked with the modified PPO enzymes of the invention.

In other embodiments, a polypeptide useful for the disclosure can be a polysaccharide degrading enzyme. Plants of this disclosure producing such an enzyme may be useful for generating, for example, fermentation feedstocks for bioprocessing. In some embodiments, enzymes useful for a fermentation process include alpha amylases, proteases, pullulanases, isoamylases, cellulases, hemicellulases, xylanases, cyclodextrin glycotransferases, lipases, phytases, laccases, oxidases, esterases, cutinases, granular starch hydrolyzing enzyme and other glucoamylases. Polysaccharide-degrading enzymes include: starch degrading enzymes such as α-amylases (EC 3.2.1.1), glucuronidases (E.C. 3.2.1.131); exo-1,4-α-D glucanases such as amyloglucosidases and glucoamylase (EC 3.2.1.3), β-amylases (EC 3.2.1.2), α-glucosidases (EC 3.2.1.20), and other exo-amylases; starch debranching enzymes, such as a) isoamylase (EC 3.2.1.68), pullulanase (EC 3.2.1.41), and the like; b) cellulases such as exo-1,4-3-cellobiohydrolase (EC 3.2.1.91), exo-1,3-β-D-glucanase (EC 3.2.1.39), β-glucosidase (EC 3.2.1.21); c) L-arabinases, such as endo-1,5-α-L-arabinase (EC 3.2.1.99), α-arabinosidases (EC 3.2.1.55) and the like; d) galactanases such as endo-1,4-β-D-galactanase (EC 3.2.1.89), endo-1,3-β-D-galactanase (EC 3.2.1.90), α-galactosidase (EC 3.2.1.22), β-galactosidase (EC 3.2.1.23) and the like; e) mannanases, such as endo-1,4-β-D-mannanase (EC 3.2.1.78), β-mannosidase (EC 3.2.1.25), α-mannosidase (EC 3.2.1.24) and the like; f) xylanases, such as endo-1,4-β-xylanase (EC 3.2.1.8), β-D-xylosidase (EC 3.2.1.37), 1,3-β-D-xylanase, and the like; and g) other enzymes such as α-L-fucosidase (EC 3.2.1.51), α-L-rhamnosidase (EC 3.2.1.40), levanase (EC 3.2.1.65), inulanase (EC 3.2.1.7), and the like. In one embodiment, the α-amylase is the synthetic α-amylase, Amy797E, described is U.S. Pat. No. 8,093,453, herein incorporated by reference in its entirety. Any such polypeptides may be stacked with the modified PPO enzymes of the invention.

Further enzymes which may be used with the disclosure include proteases, such as fungal and bacterial proteases. Fungal proteases include, but are not limited to, those obtained from Aspergillus, Trichoderma, Mucor and Rhizopus, such as A. niger, A. awamori, A. oryzae and M. miehei. In some embodiments, the polypeptides of this disclosure can be cellobiohydrolase (CBH) enzymes (EC 3.2.1.91). In one embodiment, the cellobiohydrolase enzyme can be CBH1 or CBH2. Any such polypeptides may be stacked with the modified PPO enzymes of the invention.

Other enzymes useful with the disclosure include, but are not limited to, hemicellulases, such as mannases and arabinofuranosidases (EC 3.2.1.55); ligninases; lipases (e.g., E.C. 3.1.1.3), glucose oxidases, pectinases, xylanases, transglucosidases, alpha 1,6 glucosidases (e.g., E.C. 3.2.1.20); esterases such as ferulic acid esterase (EC 3.1.1.73) and acetyl xylan esterases (EC 3.1.1.72); and cutinases (e.g. E.C. 3.1.1.74). Any such polypeptides may be stacked with the modified PPO enzymes of the invention.

In other embodiments, the modified PPO enzymes described herein may be stacked with polynucleotides that encode polypeptides which increase protein content, and/or alter seed composition and/or fatty acid content. Such sequences include, but are not limited to, sequences disclosed in PCT Appl. No. PCT/CN2022/075977 and PCT Appl. No. PCT/CN2022/075982 both filed on Feb. 11, 2022, WO2021/044027; US2020/0131524; and US2021/0403933, each of which is incorporated by reference in its entirety.

In one embodiment, the modified PPO enzymes described herein are stacked with a modified BIO3-BIO1 and/or BioA enzyme and/or a modified biotin synthesis pathway.

Suitably therefore the plant or part thereof described herein comprises modified BIO3-BIO1 and/or BioA enzyme. In one embodiment therefore, the additional modified enzyme is a modified BIO3-BIO1 and/or BioA enzyme.

Suitably therefore the plant or part thereof may further be modified to comprise a BIO3-BIO1 and/or BioA enzyme that provides the plant or part thereof with increased resistance to a compound which inhibits a BIO3-BIO1 and/or BioA enzyme and/or the biotin synthesis pathway relative to an unmodified plant. Suitably therefore the plant or part thereof may be modified to comprise a BIO3-BIO1 and/or BioA enzyme having one or more modifications which provide the plant or part thereof with a BIO3-BIO1 and/or BioA resistance trait, suitably by providing increased resistance to a compound which inhibits a biotin synthesis and ergo BIO3-BIO1 and/or BioA enzyme relative to an unmodified plant. Suitably the plant or part thereof may comprise a recombinant polynucleotide encoding a BIO3-BIO1 and/or BioA enzyme having one or more modifications. Suitably the or each modification provides the plant with an increased resistance to a compound which inhibits the BIO3-BIO1 and/or BioA enzyme relative to an unmodified plant. Suitably the or each modification provides the plant with an increased resistance to a herbicide which inhibits the BIO3-BIO1 and/or BioA enzymes and the biotin synthesis pathway. Suitably, the plant or part thereof may further be modified to comprise a BIO3-BIO1 and/or BioA enzyme as described in European patent application EP23154964.3, which is incorporated herein by reference.

### Plants or Parts Thereof

The plants of the present invention include both non-transgenic plants and transgenic plants.

By “non-transgenic plant” is intended to mean a plant lacking recombinant DNA in its genome, but containing a mutant nucleic acid molecule in the plant cell genome which has been mutated through human intervention using mutagenic techniques, such as chemical mutagenesis, gene editing or by those methods provided herein. Non-transgenic plants may encompass those plants having mutant or modified sequences as a result of natural processes, such as plants including spontaneous PPO enzymes that provide the desired resistance to compounds that inhibit PPO enzymatic activity or by the use of gene editing techniques. In specific embodiments, the non-transgenic plant comprises a modified PPO enzyme that has been altered through gene editing to comprise at least one or more of the modifications disclosed herein. Such gene editing modifications will increase the resistance of the plant to the herbicide of interest.

By “transgenic plant” is intended to mean a plant comprising recombinant DNA in its genome. As used herein, “recombinant,” when referring to nucleic acid or polypeptide, indicates that such material has been conceived of and created in the laboratory using one or more of the techniques of biotechnology, protein design, or protein engineering, such as molecular biology, protein biochemistry, bacterial transformation, plant transformation, site-directed mutagenesis, directed evolution using random mutagenesis, genome editing, gene editing, gene cloning, DNA ligation, DNA synthesis, protein synthesis, and DNA shuffling. Including for example being altered as a result of human application of a recombinant technique, such as by polynucleotide restriction and ligation, by polynucleotide overlap-extension, or by genomic insertion or transformation. A gene sequence open reading frame is recombinant if that nucleotide sequence has been removed from its natural text and cloned into any type of artificial nucleic acid vector. The term recombinant also can refer to an organism having a recombinant material, e.g., a plant that comprises a recombinant nucleic acid can be considered a recombinant plant. Such a transgenic plant can be produced by introducing recombinant DNA into the genome of the plant. When such recombinant DNA is incorporated into the genome of the transgenic plant, progeny of the plant can also comprise the recombinant DNA. A progeny plant that comprises at least a portion of the recombinant DNA of at least one progenitor transgenic plant is also a transgenic plant.

As used herein, “heterologous” in reference to a polypeptide or polynucleotide sequence is a sequence that originates, for example, from a cell or an organism from a foreign species. Alternatively, if the sequence originates from the same species, it is derived from a cell or organism having a different genetic background; or if from the same genetic background, it is substantially modified from its native form in composition and/or genomic locus by deliberate human intervention. As such, heterologous sequences are in a configuration not found in nature.

The term “spontaneous mutant” refers to mutants or variants that arise from the parent strain without the intentional use of mutagens i.e. they are considered as not genetically modified (non-GMO). Spontaneous mutants in respect of plants may also be known as sports, breaks, or chimeras.

Suitably the plant or part thereof of the invention is transgenic. In other embodiments, the plant or plant part thereof of the invention is non-transgenic and comprises a gene edit that increases the plant's or plant part's tolerance to a herbicide of interest.

In one embodiment, therefore, the plant or part thereof comprises a recombinant polynucleotide encoding a modified PPO enzyme. In some embodiments, the plant or part thereof comprises a polynucleotide encoding a modified or mutated PPO enzyme. Suitably the polynucleotide encoding the modified PPO enzyme may comprise one or more modifications. Suitably therefore the polynucleotide may be operable to express a modified PPO enzyme having one or more modifications or mutations. Suitably wherein the expression of said polynucleotide provides or confers to the plant or part thereof increased resistance to a compound which inhibits PPO enzymatic activity. Suitably the or each modification in the modified PPO enzyme provides increased resistance to a compound which inhibits PPO enzymatic activity. Suitable PPO modifications are defined herein and can include any one of SEQ ID NO: 1, 2, 4-151, 153-302 and 305-336 or active variants thereof as described elsewhere herein. For example, such PPO modifications are defined herein and can include any one of SEQ ID NO: 37-39, 58, 59, 97-99, 118, 119, 125-137, 139-151, 188-190, 209, 210, 248-250, 269, 270, 277-288 or 291-302 or active variants thereof as described elsewhere herein. In specific embodiments, the modified or mutated PPO enzyme is encoded by a recombinant polynucleotide stably integrated into the plant's genome. Alternatively, the modified or mutated PPO enzyme is encoded by a polynucleotide, suitably a gene, comprising a non-transgenic modification, such as an edit, within the genome of the plant.

In one embodiment, the plant or part thereof may be modified to comprise a modified PPO enzyme wherein the modified PPO enzyme is overexpressed and wherein the modified PPO enzyme comprises one or more mutations provided herein which provide the plant or part thereof with increased resistance to a compound which inhibits PPO enzymatic activity relative to an unmodified or control PPO enzyme or plant. Suitable modified PPOs can include any one of SEQ ID NO: 1, 2, 4-151, 153-302 and 305-336. For example, modified PPOs can include any one of SEQ ID NO: 37-39, 58, 59, 97-99, 118, 119, 125-137, 139-151, 188-190, 209, 210, 248-250, 269, 270, 277-288 or 291-302 or active variants thereof as described elsewhere herein.

In one embodiment, the plant has been transformed with said recombinant polynucleotide.

Suitable means of transformation are described hereinbelow.

The transformed parts of plants, transformed plant cells or a transformed plant protoplasts as described herein may be regenerated to produce a modified plant as described herein. When adequate numbers of transformed cells or protoplasts containing a recombinant modified PPO enzyme are obtained, the cells can be cultured, then regenerated into whole plants. “Regeneration” refers to the process of growing a plant from a plant cell (for example, plant protoplast or explant). Such regeneration techniques rely on manipulation of certain phytohormones in a tissue culture growth medium, typically relying on a biocide and/or herbicide marker that has been introduced together with the desired nucleotide sequences. Choice of methodology for the regeneration step is not critical. See, for example, Ammirato et al., Handbook of Plant Cell Culture—Crop Species. Macmillan Publ. Co. (1984); Shimamoto et al., Nature 338:274-276 (1989); Fromm, UCLA Symposium on Molecular Strategies for Crop Improvement, Apr. 16-22, 1990. Keystone, Colo. (1990); Vasil et al., Bio/Technology 8:429-434 (1990); Vasil et al., Bio/Technology 10:667-674 (1992); Hayashimoto, Plant Physiol. 93:857-863 (1990); and Datta et al., Bio-technology 8:736-740 (1990). Such regeneration techniques are described generally in Klee et al., Ann. Rev. Plant Phys. 38:467-486 (1987).

The present invention may be for use with any plant species and the progeny thereof, including, but not limited to, monocots and dicots.

Examples of plant species of interest include, but are not limited to, corn or maize (Zea mays), Brassica sp. (e.g., B. napus, B. rapa, B. juncea), including those Brassica species useful as sources of seed oil, alfalfa (Medicago sativa), rice (Oryza sativa), rye (Secale cereale), Sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum, T. turgidum ssp. durum), soybean (Glycine max), tobacco (Nicotiana tabacum), potato (Solarium tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), Citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), Macadamia (Macadamia integrifolia), almond (Primus amygdalus), sugar beets (Beta vulgaris), sugarcane (Saccharum spp.), oats (Avena sativa), barley (Hordeum vulgare), vegetables, ornamentals, and conifers.

In one embodiment, plants of the present invention are crop plants (for example, sunflower, Brassica sp., cotton, sugar, beet, soybean, peanut, alfalfa, safflower, tobacco, corn, rice, wheat, rye, barley triticale, Sorghum, millet, etc.).

In another embodiment, plants of the present invention may also include various types of cover crop plants. Exemplary cover crop plants include, but are not limited to, Brassica sp. (e.g., B. carinata, B. napus, B. rapa, B. hirta, B. juncea, B. nigra), radish (Raphanus sativus), Camelina sp. (e.g., C. sativa), pennycress (Thlaspi arvense), clover (Trifolium sp., e.g., T. incarnatum, T. pratense, T. repens, T. subterraneum), field peas (Pisum sativum), Vicia sp. (e.g., V. villosa, V. lutea, V. nigricans, V. sativa), rye (Secale cereale), barley (Hordeum vulgare), winter wheat (Triticum aestivum), oats (Avena sativa), annual ryegrass (Lolium multiflorum), buckwheat (Fagopyrum esculentum), Sinapsis alba, alfalfa (Medicago sativa) As used herein, the terms “progeny” and “progeny plant” refer to a plant generated from a vegetative or sexual reproduction from one or more parent plants. A progeny plant may be obtained by cloning or selfing a single parent plant, or by crossing two parental plants.

As used herein unless clearly indicated otherwise, the term “plant” is intended to mean a plant at any developmental stage, as well as any part or parts of a plant that may be attached to or separate from a whole intact plant. Such parts of a plant include, but are not limited to, seed, organs, tissues, and cells of a plant including, plant calli, plant clumps, plant protoplasts and plant cell tissue cultures from which plants can be regenerated. Examples of particular plant parts include a stem, a leaf, a root, an inflorescence, a flower, a floret, a fruit, a pedicle, a peduncle, a stamen, an anther, a stigma, a style, an ovary, a petal, a sepal, a carpel, a root tip, a root cap, a root hair, a leaf hair, a seed hair, a pollen grain, a microspore, an embryos, an ovule, a cotyledon, a hypocotyl, an epicotyl, xylem, phloem, parenchyma, endosperm, a companion cell, a guard cell, and any other known organs, tissues, and cells of a plant. Furthermore, it is recognized that a seed is a plant part.

A “plant cell” is a structural and physiological unit of a plant, comprising a protoplast and a cell wall. The plant cell may be in the form of an isolated single cell or a cultured cell, or as a part of a higher organized unit such as, for example, plant tissue, a plant organ, or a whole plant. A “plant part” is a distinct and visibly structured and differentiated part of a plant such as a root, stem, leaf, flower bud, or embryo.

The plants, progeny thereof or parts thereof of the invention express at least one of a modified PPO enzyme as disclosed herein. Expression of the enzymes, polynucleotides encoding said enzymes, or expression vectors of the invention provides a plant that is at least partially resistant to compounds which inhibit PPO enzymatic activity, such as those herbicides described herein. For example, the plants, progeny thereof or parts thereof of the invention have increased resistance to a herbicide which inhibits PPO enzymatic activity.

The increase in resistance may be determined by comparison to a wild-type or control plant as described herein. For example, a plant that has not been modified to include or express the modified PPO enzymes, polynucleotides encoding them, or expression vectors of the invention.

### Methods of Producing Modified Plants or Increasing Resistance of a Plant

Provided herein are methods of conferring increased resistance to compounds which inhibit PPO enzymes (i.e. PPO enzymatic activity) to a plant or part thereof by modifying the plant to comprise a modified PPO enzyme as disclosed herein which provides said resistance.

Suitably such methods may include (i) a method of producing a modified plant or part thereof having an increased resistance to a compound which inhibits PPO enzymatic activity, and (ii) a method of increasing the resistance of a plant or part thereof to a compound which inhibits PPO enzymatic activity.

Suitably either method comprises a step of modifying the plant or part thereof to comprise a modified PPO enzyme that provides the increased resistance.

Suitably modifying the plant may comprise providing the plant or part thereof with a modified PPO enzyme having one or more modifications, wherein the or each modification provides the increased resistance.

Suitably such steps may comprise providing the plant or part thereof with a recombinant polynucleotide encoding a modified PPO enzyme as described herein.

Suitably providing may comprise introducing the recombinant polynucleotide encoding a modified PPO enzyme as provided herein into the plant or part thereof, or introducing the modified PPO protein into the plant or part thereof. Suitably the modified PPO enzyme may be introduced into a plant or part thereof by introducing a polynucleotide of the invention which encodes a modified PPO enzyme. Thus, plants of the invention may be referred to as modified or transgenic plants.

Suitably introducing the modified PPO enzyme into a plant or part thereof may be carried out by transforming the plant or part thereof with a recombinant polynucleotide encoding a modified PPO enzyme.

Suitably the recombinant polynucleotide may further encode a transit peptide, such as a mitochondrial or chloroplast transit peptide. Suitably as a fusion with the modified PPO enzyme. Suitably therefore the recombinant polynucleotide may comprise a chimeric polynucleotide encoding a modified PPO as described above and a transit peptide operably linked thereto, suitably a mitochondrial or chloroplast transit peptide operably linked thereto.

Suitable transit peptides are described elsewhere herein.

Suitably the recombinant polynucleotide may be part of an expression construct, or comprised in an expression vector. Suitably the expression construct or vector may comprise one or more expression elements such as a promoter, as is described elsewhere herein. Suitably therefore the methods may comprise providing, introducing or transforming the plant or part thereof with an expression construct or expression vector comprising a polynucleotide encoding a modified PPO enzyme, suitably which may be a recombinant polynucleotide.

Suitably the methods may further comprise a step of inducing expression of the recombinant polynucleotide to produce the modified PPO enzyme provided herein in the plant or part thereof. Suitably inducing expression of the polynucleotide may comprise contacting the plant or part thereof with an inducer. Suitably in such embodiments the polynucleotide encoding the modified PPO enzyme may be under the control of an inducible promoter, suitably therefore the expression construct or vector may comprise an inducible promoter operably linked to the polynucleotide encoding the modified PPO enzyme. Suitable inducible promoters and inducers are well known in the art.

In addition to or alternatively a plant may be modified by in situ editing of the endogenous genetic material in order to provide a gene that expresses a modified PPO enzyme which provides increased resistance to compounds which inhibit PPO enzymatic activity.

Suitably in such embodiments, a plant may be provided with the components of a gene editing system for modifying an endogenous gene sequence of the plant encoding PPO enzyme at one or more positions to produce a modified gene sequence encoding a modified PPO enzyme which provides increased resistance to a compound which PPO enzymatic activity. Suitably the plant may be transformed with one or more polynucleotides encoding a gene editing system for modifying an endogenous gene sequence of the plant encoding PPO enzyme at one or more positions to produce a modified gene sequence encoding modified PPO enzyme which provides increased resistance to a compound which inhibits PPO enzymatic activity. Thus providing a plant or part thereof that expresses a modified PPO enzyme which provides increased resistance to a compound which inhibits PPO enzymatic activity.

An endogenous PPO encoding gene sequence may be edited in situ by way of gene editing techniques in order to provide a modified PPO enzyme that is at least partially resistant to compound that inhibits PPO enzymatic activity, such as those described herein and as such a modified plant as described herein. Such genome editing and/or mutagenesis technologies are well known in the art. As well, introduction may be accomplished by any manner known in the art, including: introgression, transgenic, or site-directed nucleases (SDN). Particularly, the modification to the gene sequence is introduced by way of site-directed nuclease (SDN). More particularly, the SDN is selected from: meganuclease, zinc finger, transcription activator-like effector nucleases system (TALEN) or Clustered Regularly Interspaced Short Palindromic Repeats system (CRISPR) system. SDN is also referred to as “genome editing”, or genome editing with engineered nucleases (GEEN). This is a type of genetic engineering in which DNA is inserted, deleted or replaced in the genome of an organism using engineered nucleases that create site-specific double-strand breaks (DSBs) at desired locations in the genome. The induced double-strand breaks are repaired through nonhomologous end-joining (NHEJ) or homologous recombination (HR), resulting in targeted mutations (‘edits’). Particularly SDN may comprises techniques such as: Meganucleases, Zinc finger nucleases (ZFNs), Transcription Activator-Like Effector-based Nucleases (TALEN) (Feng et al. 2013 Cell Res. 23, 1229-1232, Sander & Joung Nat. Biotechnol. 32, 347-355 2014), and the Clustered Regularly Interspaced Short Palindromic Repeats (CRISPR-Cas) system. Gene editing may also be achieved by SDN-2. SDN-2 is similar to SDN, but also provides a small nucleotide template complementary to the area of the break.

The template contains one or more sequences modifications to the genomic DNA which are incorporated to create the mutation to the target gene. In one embodiment, the gene editing system may include a CRISPR-Cas system.

As used herein, the term “guide RNA” or “gRNA” generally refers to an RNA molecule (or a group of RNA molecules collectively) that can bind to a CRISPR system effector, such as a Cas or a Cpf 1 protein, and aid in targeting the Cas or Cpf1 protein to a specific location within a target polynucleotide (e.g., a DNA). A guide RNA of the invention can be an engineered, single RNA molecule (sgRNA), where for example the sgRNA comprises a crRNA segment and optionally a tracrRNA segment. A guide RNA of the invention can also be a dual-guide system, where the crRNA and tracrRNA molecules are physically distinct molecules which then interact to form a duplex for recruitment of a CRISPR system effector, such as Cas9, and for targeting of that protein to the target polynucleotide.

As used herein, the term “crRNA” or “crRNA segment” refers to an RNA molecule or to a portion of an RNA molecule that includes a polynucleotide targeting guide sequence, a stem sequence involved in protein-binding, and, optionally, a 3-overhang sequence. The polynucleotide targeting guide sequence is a nucleic acid sequence that is complementary to a sequence in a target DNA (for example a gene encoding a PPO enzyme). This polynucleotide targeting guide sequence is also referred to as the “protospacer”. In other words, the polynucleotide targeting guide sequence of a crRNA molecule interacts with a target DNA in a sequence-specific manner via hybridization (i.e., base pairing). As such, the nucleotide sequence of the polynucleotide targeting guide sequence of the crRNA molecule may vary and determines the location within the target DNA that the guide RNA and the target DNA will interact.

The polynucleotide targeting guide sequence of a crRNA molecule can be modified (e.g., by genetic engineering) to hybridize to any desired sequence within a target DNA. The polynucleotide targeting guide sequence of a crRNA molecule of the invention can have a length from about 12 nucleotides to about 100 nucleotides. For example, the polynucleotide targeting guide sequence of a crRNA can have a length of from about 12 nucleotides (nt) to about 80 nt, from about 12 nt to about 50 nt, from about 12 nt to about 40 nt, from about 12 nt to about 30 nt, from about 12 nt to about 25 nt, from about 12 nt to about 20 nt, or from about 12 nt to about 19 nt. For example, the polynucleotide targeting guide sequence of a crRNA can have a length of from about 17 nt to about 27 nts.

For example, the polynucleotide targeting guide sequence of a crRNA can have a length of from about 19 nt to about 20 nt, from about 19 nt to about 25 nt, from about 19 nt to about 30 nt, from about 19 nt to about 35 nt, from about 19 nt to about 40 nt, from about 19 nt to about 45 nt, from about 19 nt to about 50 nt, from about 19 nt to about 60 nt, from about 19 nt to about 70 nt, from about 19 nt to about 80 nt, from about 19 nt to about 90 nt, from about 19 nt to about 100 nt, from about 20 nt to about 25 nt, from about 20 nt to about 30 nt, from about 20 nt to about 35 nt, from about 20 nt to about 40 nt, from about 20 nt to about 45 nt, from about 20 nt to about 50 nt, from about 20 nt to about 60 nt, from about 20 nt to about 70 nt, from about 20 nt to about 80 nt, from about 20 nt to about 90 nt, or from about 20 nt to about 100 nt. The nucleotide sequence of the polynucleotide targeting guide sequence of a crRNA can have a length at least about 12 nt. In some embodiments, the polynucleotide targeting guide sequence of a crRNA is 20 nucleotides in length. In some embodiments, the polynucleotide targeting guide sequence of a crRNA is 19 nucleotides in length.

The present invention also provides a guide RNA comprising an engineered crRNA, wherein the crRNA comprises a bait RNA segment capable of hybridizing to a genomic target sequence. This engineered crRNA may be a physically distinct molecule, as in a dual-guide system.

As used herein, the term “tracrRNA” or “tracrRNA segment” refers to an RNA molecule or portion thereof that includes a protein-binding segment (e.g., the protein-binding segment is capable of interacting with a CRISPR-associated protein, such as a Cas9). The present invention also provides a guide RNA comprising an engineered tracrRNA, wherein the tracrRNA further comprises a bait RNA segment that is capable of binding to a donor DNA molecule. The engineered tracrRNA may be a physically distinct molecule, as in a dual-guide system, or may be a segment of a sgRNA molecule.

The guide RNA, either as a sgRNA or as two or more RNA molecules, does not contain a tracrRNA, as it is known in the art that some CRISPR-associated nucleases, such as Cpf1 (also known as Casl2a), do not require a tracrRNA for its RNA-mediated endonuclease activity (Qi et al., (2013), Cell, 152: 1173-1183; Zetsche et al., (2015), Cell 163: 759-771).

Such a guide RNA of the invention may comprise a crRNA with the bait RNA operably linked at the 5′ or 3′ end of the crRNA. Cpf1 also has RNase activity on its cognate pre-crRNA (Fonfara et al., (2016), Nature, doi.org/10.1038/naturel7945).

A guide RNA of the invention may comprise multiple crRNAs which the Cpf1 possesses to mature crRNAs. Each of these crRNAs may be operably linked to a bait RNA. At least one of these crRNAs may be operably linked to a bait RNA. The bait RNA may be specific to a sequence of interest (SOI), or it may be a “universal” bait, which has a corresponding “universal” prey sequence on the donor DNA molecule.

The present invention also provides a polynucleotide comprising a sequence encoding a guide RNA of the invention. The polynucleotide may be a DNA or an RNA molecule. The polynucleotide molecule may be circularized or linear. The polynucleotide may be single stranded, partially double-stranded, or double-stranded. The polynucleotide may be complexed with at least one polypeptide. The polypeptide may have a nucleic acid recognition or nucleic acid binding domain. The polypeptide may be a shuttle for mediating delivery of, for example, a polynucleotide of the invention, a nuclease, and optionally a donor molecule. The polypeptide may be a Feldan Shuttle (U.S. Patent Publication No. 20160298078, herein incorporated by reference). The polynucleotide may comprise an expression cassette capable of driving the expression of the polynucleotide. The polynucleotide may further comprise additional expression cassettes, capable of expressing, for example, a nuclease such as a CRISPR-associated nuclease.

Suitably therefore the plant or part thereof may be provided with, specifically transformed with, a Cas enzyme, or one or more polynucleotides encoding a Cas enzyme, and a polynucleotide sequence encoding a guide RNA. Suitably the guide RNA is complementary to a PPO gene or regulatory sequences thereof, in the plant or part thereof. Suitably the guide RNA is operable to target the Cas enzyme to edit the PPO gene and provide the plant or part thereof with increased resistance to a compound which inhibits PPO enzymatic activity pathway relative to an unmodified plant.

In addition to or alternatively a plant may be modified by providing the plant or part thereof with one or more regulatory RNA sequences operable to target a gene that encodes a PPO enzyme or a regulatory sequence thereof. Suitably the plant or part thereof may be transformed with one or more regulatory RNA sequences operable to target a gene that encodes a PPO enzyme or a regulatory sequence thereof. Suitably the regulatory RNA sequence may be complementary to, and bind to, a gene that encodes a PPO enzyme or a regulatory sequence thereof in the plant or part thereof, and act to provide increased resistance to compounds which inhibit PPO enzymatic activity.

“Transformation” refers to a process of introducing an exogenous nucleic acid molecule (for example, a recombinant polynucleotide) into a cell or protoplast and that exogenous nucleic acid molecule is incorporated into a host cell genome or an organelle genome (for example, chloroplast or mitochondria) or is capable of autonomous replication. “Transformed” or “transgenic” refers to a cell, tissue, organ, or organism into which a foreign nucleic acid, such as an expression vector or recombinant nucleic acid molecule has been introduced. The nucleic acid molecule can be stably integrated into the genome of the host or the nucleic acid molecule can also be present as an extrachromosomal molecule. Such an extrachromosomal molecule can be auto-replicating. The nucleic acid molecule can also be introduced into the genome of the chloroplast or the mitochondria of a plant cell.

Methods of transformation of plant cells or tissues include, but are not limited to, Agrobacterium mediated transformation method and the Biolistics or particle-gun mediated transformation method. Suitable plant transformation vectors for the purpose of Agrobacterium mediated transformation include-those elements derived from a tumor inducing (Ti) plasmid of Agrobacterium tumefaciens, for example, right border (RB) regions and left border (LB) regions, and others disclosed by Herrera-Estrella et al., Nature 303:209 (1983); Bevan, Nucleic Acids Res. 12:8711-8721 (1984); Klee et al., Bio-Technology 3(7):637-642 (1985). In addition to plant transformation vectors derived from the Ti or root-inducing (Ri) plasmids of Agrobacterium, alternative methods can be used to insert the nucleic acid molecules of this invention into plant cells. Such methods may involve, but are not limited to, for example, the use of liposomes, electroporation, chemicals that increase free DNA uptake, free DNA delivery via microprojectile bombardment, and transformation using viruses or pollen.

A “transgenic” or “transformed” cell or plant also includes progeny of the cell or plant and progeny produced from a breeding program employing such a “transgenic” plant as a parent in a cross and exhibiting an altered phenotype resulting from the presence of the foreign nucleic acid molecule.

The transgenic plants may be homozygous for the polynucleotide encoding a modified PPO enzyme described herein (i.e. those that contain two added genes encoding the enzyme at the same position on each chromosome of the chromosome pair). Homozygous transgenic plants may be obtained by crossing (self-pollinating) independent transgenic plant isolates containing a single added gene, germinating some of the resulting seeds, and transforming the resulting plant with the target gene.

### Breeding Plants

The development or regeneration of transgenic plants containing a nucleic acid molecule that encodes recombinant modified PPO enzymes of the invention is well known in the art. The regenerated plants may be self-pollinated to provide homozygous transgenic plants, as discussed above. Otherwise, pollen obtained from the regenerated plants is crossed to seed-grown plants of agronomically important lines. Conversely, pollen from plants of these important lines is used to pollinate regenerated plants.

The at least partially resistant plants and progeny of such plants described herein (such as transformed, modified or transgenic plants described herein), can be used in methods for preparing at least partially resistant plants, plants having increased tolerance to compounds which inhibit PPO enzymatic activity, and seeds of such plants. Thus, for example, the plants exemplified herein may be used in breeding programs to develop additional at least partially herbicide resistant plants, such as commercial varieties of such plants. In accordance with such methods, a first parent plant may be used in crosses with a second parent plant, where at least one of the first or second parent plants contains a modified PPO enzyme as described herein. One application of the process is in the production of F1 hybrid plants. Another aspect of this process is that the process can be used for the development of novel parent, dihaploid or inbred lines. For example, a plant line as described herein could be crossed to any second plant, and the resulting hybrid progeny each selfed and/or sibbed for about 5 to 7 or more generations, thereby providing a large number of distinct, parent lines. These parent lines could then be crossed with other lines and the resulting hybrid progeny analyzed for beneficial characteristics. In this way, novel lines conferring desirable characteristics could be identified. Various breeding methods may be used in the methods, including haploidy, pedigree breeding, single-seed descent, modified single seed descent, recurrent selection, and backcrossing.

The plants and progeny thereof may display a synergistic effect rather than additive effect of tolerance to compounds which inhibit PPO enzymatic activity, whereby the level of tolerance in the plants and the progeny thereof comprising multiple mutations is greater than the combined tolerance of plants comprising a single modified PPO enzyme.

Plant lines containing the modified PPO enzymes of the present invention can be crossed by either natural or mechanical techniques. Mechanical pollination can be effected either by controlling the types of pollen that can be transferred onto the stigma or by pollinating by hand.

Any breeding method may be used in the methods of the present invention. In one example, the resistant plants of the present invention may be bred using a haploid method. In such methods, parents having the genetic basis for the desired complement of characteristics are crossed in a simple or complex cross. Crossing (or cross-pollination) refers to the transfer of pollen from one plant to a different plant. Progeny of the cross are grown and microspores (immature pollen grains) are separated and filtered, using techniques known to those skilled in the art [(e.g. Swanson, E. B. et al, (1987) Plant Cell Reports, 6: 94-97, “Efficient isolation of microspores and the production of microspore-derived embryos in Brassica napus, L.; and Swanson, E. B., (1990) Microspore culture in Brassica, pp. 159-169 in Methods in Molecular Biology, vol. 6, Plant Cell and Tissue Culture, Humana Press]. These microspores exhibit segregation of genes. The microspores are cultured in the presence of an appropriate AHAS-inhibitor herbicide, such as imazethapyr (e.g. PURSUIT™) or imazamox (e.g. SOLO™, BEYOND™, and RAPTOR™) or a 50/50 mix of imazethapyr and imazamox (e.g. ODYSSEY™), which kills microspores lacking the mutations responsible for resistance to the herbicide. Microspores carrying the genes responsible for resistance to the herbicide survive and produce embryos, which form haploid plants. Their chromosomes are then doubled to produce doubled haploids.

Other breeding methods may also be used in accordance with the present invention. For example, pedigree breeding may be used for the improvement of largely self-pollinating crops such as Brassica and canola. Pedigree breeding starts with the crossing of two genotypes, each of which may have one or more desirable characteristics that is lacking in the other or which complements the other. If the two original parents do not provide all of the desired characteristics, additional parents can be included in the crossing plan. These parents may be crossed in a simple or complex manner to produce a simple or complex F1. An F2 population is produced from the F1 by selfing one or several F1 plants, or by intercrossing two F1's (i.e., sib mating). Selection of the best individuals may begin in the F2 generation, and beginning in the F3 the best families, and the best individuals within the best families are selected. Replicated testing of families can begin in the F4 generation to improve the effectiveness of selection for traits with low heritability. At an advanced stage of inbreeding (i.e., F6 and F7), the best lines or mixtures of phenotypically similar lines may be tested for potential release as new cultivars. However, the pedigree method is more time-consuming than the haploidy method for developing improved plants which are at least partially resistant to PPO enzyme inhibiting compounds, because the plants exhibit segregation for multiple generations, and the recovery of desirable traits is relatively low.

The single seed descent (SSD) procedure may also be used to breed improved varieties.

The SSD procedure in the strict sense refers to planting a segregating population, harvesting a sample of one seed per plant, and using the population of single seeds to plant the next generation. When the population has been advanced from the F2 to the desired level of inbreeding, the plants from which lines are derived will each trace to different F2 individuals. The number of plants in a population declines each generation due to failure of some seeds to germinate or some plants to produce at least one seed. As a result, not all of the plants originally sampled in the F2 population will be represented by a progeny when generation advance is completed.

In a multiple-seed procedure, canola breeders commonly harvest one or more pods from each plant in a population and thresh them together to form a bulk. Part of the bulk is used to plant the next generation and part is put in reserve. The procedure has been referred to as modified single-seed descent or the pod-bulk technique. The multiple-seed procedure has been used to save labour at harvest. It is considerably faster to thresh pods with a machine than to remove one seed from each by hand for the single-seed procedure. The multiple-seed procedure also makes it possible to plant the same number of seeds of a population each generation of inbreeding. Enough seeds are harvested to make up for those plants that did not germinate or produce seed.

Backcross breeding can be used to transfer a gene or genes for a simply inherited, highly heritable trait from a source variety or line (the donor parent) into another desirable cultivar or inbred line (the recurrent parent). After the initial cross, individuals possessing the phenotype of the donor parent are selected and are repeatedly crossed (backcrossed) to the recurrent parent. When backcrossing is complete, the resulting plant is expected to have the attributes of the recurrent parent and the desirable trait transferred from the donor parent.

Improved varieties may also be developed through recurrent selection. In this method, genetically variable population of heterozygous individuals is either identified or created by intercrossing several different parents. The best plants are selected based on individual superiority, outstanding progeny, or excellent combining ability. The selected plants are intercrossed to produce a new population in which further cycles of selection are continued.

At least partially resistant plants can be produced by cross-pollinating a first plant with a second plant and allowing the pollen acceptor plant (can be either the first or second plant) to produce seed from this cross pollination. Seeds and progeny plants generated therefrom can have the mutation crossed into the genome of the seed and/or progeny plants. The pollen-acceptor plant can be either the first or second plant. The first plant comprises a nucleic acid encoding a modified PPO enzyme as disclosed herein. The second plant can be any compatible plant and may comprise a second same or different modified PPO enzyme. The first and second enzymes may comprise a nucleic acid encoding the same or different amino acid substitution(s) relative to a wild-type modified PPO enzyme. Seeds or progeny plants arising from the cross which comprise one or two nucleic acids encoding modified PPO enzymes can be selected.

When the first and second plants are homozygous for the first and second nucleic acid molecules, respectively, each of the resulting progeny plants comprises one copy of each of the first and second nucleic acid molecules and the selection step can be omitted. When at least one of the first and second plants is heterozygous, progeny plants comprising both nucleic acid molecules can be selected, for example, by analyzing the DNA of progeny plants to identify progeny plants comprising both the first and second nucleic acid molecules or by testing the progeny plants for increased herbicide tolerance.

Descendent and/or progeny plants may be evaluated for the nucleic acid molecules of the present invention by any method to determine the presence of a specific modified PPO nucleic acid or enzyme.

Therefore, also provided herein are methods of selecting a plant or part thereof that includes a modified PPO enzyme of the invention, or a nucleic acid or expression vector encoding it, by exposing the plant or part thereof to an effective amount of a compound which inhibits a PPO enzymatic activity sufficient to prevent or reduce the growth of a plant that does not include at least a modified PPO enzyme of the invention, or a nucleic acid or expression vector encoding it. It may then be determined by the methods described herein whether the plant has been affected (e.g. has reduced growth or reduced damage) by the compound.

Plants that are unaffected by the compound may then be selected.

Methods of determining whether a plant includes the modified PPO enzyme of the invention, or a nucleic acid or expression vector encoding it and/or is affected by a compound which inhibits PPO enzymatic activity include phenotypic evaluations, genotypic evaluations, or combinations thereof. The progeny plants may be evaluated in subsequent generations for resistance to the compound, and other desirable traits. Resistance to compounds which inhibit PPO enzymatic activity may be evaluated by exposing plants to one or more appropriate compounds and evaluating injury. Some traits, such as lodging resistance and plant height, may be evaluated through visual inspection of the plants, while earliness of maturity may be evaluated by a visual inspection of seeds within pods (siliques). Other traits, such as oil percentage, protein percentage, and total glucosinolates of seeds may be evaluated using techniques such as Near Infrared Spectroscopy and/or liquid chromatography and/or gas chromatography.

Plants of the present invention can also be identified using any genotypic analysis method. Genotypic evaluation of the plants includes using techniques such as Isozyme Electrophoresis, Restriction Fragment Length Polymorphisms (RFLPs), Randomly Amplified Polymorphic DNAs (RAPDs), Arbitrarily Primed Polymerase Chain Reaction (AP-PCR), Allele-specific PCR (AS-PCR), DNA Amplification Fingerprinting (DAF), Sequence Characterized Amplified Regions (SCARs), Amplified Fragment Length Polymorphisms (AFLPs), Simple Sequence Repeats (SSRs) which are also referred to as “Microsatellites”. Additional compositions and methods for analyzing the genotype of the plants provided herein include those methods disclosed in U.S. Publication No. 2004/0171027, U.S. Publication No. 2005/02080506, and U.S. Publication No. 2005/0283858, the entireties of which are hereby incorporated by reference.

Evaluation and manipulation (through exposure to one or more appropriate compounds which inhibit PPO enzymatic activity) may occur over several generations. The performance of the new lines may be evaluated using objective criteria in comparison to check varieties. Lines showing the desired combinations of traits are either crossed to another line or self-pollinated to produce seed.

“Sequencing DNA” refers to determining the nucleic acid sequence of a piece of DNA, e.g. of a gene. Standard methods and commercial services are known in the art. Basic methods for DNA sequencing include the Maxam-Gilbert method and the chain termination method. High-throughput techniques have also been developed and may be used in the method of the present invention. These high-throughput techniques include, but are not limited to, Massively parallel signature sequencing (MPSS), Polony sequencing, 454 pyrosequencing, Illumina (Solexa) sequencing, Combinatorial probe anchor synthesis (cPAS), SOLiD sequencing, Ion Torrent semiconductor sequencing, DNA nanoball sequencing, Heliscope single molecule sequencing, Single molecule real time (SMRT) sequencing and Nanopore DNA sequencing.

Sequencing may be carried out using primers that are capable of binding to an isolated polynucleotide of the invention. For example, primers that are complimentary to at least a portion of an isolated polynucleotide of the invention.

As used herein, the term “primer” refers to an oligonucleotide which is capable of annealing to a polynucleotide target and serving as a point of initiation of DNA synthesis when placed under conditions in which synthesis of a primer extension product is induced (e.g., in the presence of nucleotides and an agent for polymerization such as DNA polymerase and at a suitable temperature and pH). A primer (in some examples an extension primer and in some examples an amplification primer) may be single stranded for maximum efficiency in extension and/or amplification. The primer may be an oligodeoxyribonucleotide. A primer is typically sufficiently long to prime the synthesis of extension and/or amplification products in the presence of the agent for polymerization. The minimum length of the primer can depend on many factors, including, but not limited to temperature and composition (A/T vs. G/C content) of the primer. In the context of amplification primers, these are typically provided as a pair of bi-directional primers consisting of one forward and one reverse primer or provided as a pair of forward primers as commonly used in the art of DNA amplification such as in PCR amplification.

As such, it will be understood that the term “primer,” as used herein, can refer to more than one primer, particularly in the case where there is some ambiguity in the information regarding the terminal sequence(s) of the target region to be amplified. Hence, a “primer” can include a collection of primer oligonucleotides containing sequences representing the possible variations in the sequence or includes nucleotides which allow a typical base pairing. Primers can be prepared by any suitable method known in the art. Methods for preparing oligonucleotides of specific sequence are known in the art, and include, for example, cloning and restriction of appropriate sequences and direct chemical synthesis. Chemical synthesis methods can include, for example, the phospho di- or tri-ester method, the diethylphosphoramidate method and the solid support method disclosed in U.S. Pat. No. 4,458,066.

Primers can be labelled, if desired, by incorporating detectable moieties by for instance spectroscopic, fluorescence, photochemical, biochemical, immunochemical, or chemical moieties. Primers diagnostic (i.e. able to identify or select based on presence of modified PPO encoding nucleic acids and the modified PPO enzymes thereof as described herein) for resistance to compounds which inhibit PPO enzymatic activity can be created by any known methods. The PCR method is well described in handbooks and known to the skilled person. After amplification by PCR, target polynucleotides can be detected by hybridization with a probe polynucleotide, which forms a stable hybrid with the target sequence under stringent to moderately stringent hybridization and wash conditions. If it is expected that the probes are essentially completely complementary (i.e., about 99% or greater) to the target sequence, stringent conditions can be used.

If some mismatching is expected, for example if variant polynucleotides are expected with the result that the probe will not be completely complementary, the stringency of hybridization can be reduced. In some examples, conditions are chosen to rule out non-specific/adventitious binding. Conditions that affect hybridization, and that select against non-specific binding are known in the art, and are described in, for example, Sambrook & Russell (2001) Molecular Cloning: A Laboratory Manual, Third Edition, Cold Spring Harbor Laboratory Press, Cold Spring Harbor, New York, United States of America. Generally, lower salt concentration and higher temperature hybridization and/or washes increase the stringency of hybridization conditions.

### Seeds

Also included herein are seeds that are capable of producing a plant or part thereof of the invention. Suitably seeds that comprise a modified PPO enzyme provided herein, or a polynucleotide or expression vector encoding a modified PPO enzyme provided herein, which provides increased resistance to a compound which inhibits PPO enzymatic activity relative to an unmodified plant.

The term “seed” embraces seeds and plant propagules of all kinds including but not limited to true seeds, seed pieces, suckers, corms, bulbs, fruit, tubers, grains, cuttings, cut shoots and the like.

Seeds may be treated or untreated seeds. For example, the seeds can be treated to improve germination, for example, by priming the seeds, or by disinfection to protect against seed-borne pathogens. In another example, seeds can be coated with any available coating to improve, for example, plantability, seed emergence, and protection against seed-borne pathogens. Seed coating can be any form of seed coating including, but not limited to pelleting, film coating, and encrustments.

The seed may be germinated and used to produce or grow a plant or part thereof of the invention. That is a plant or part thereof including a modified PPO enzyme which provides increased resistance to a compound which inhibits PPO enzymatic activity relative to an unmodified plant.

Also provided herein is a container including seeds of the invention. A container of seeds may contain any number, weight or volume of seeds. For example, a container can contain at least, or greater than, about 10, 25, 50, 75, 100, 200, 300, 400, 500, 600, 700, 800, 900, 1000, 1500, 2000, 2500, 3000, 3500, 4000, 4500, 5000 or more seeds. Alternatively, the container can contain at least, or greater than, about 1 ounce, 5 ounces, 10, ounces, 1 pound, 2 pounds, 3 pounds, 4 pounds, 5 pounds or more seeds.

Containers of plant seeds may be any container available in the art. By way of non-limiting example, a container may be a box, a bag, a packet, a pouch, a tape roll, a pail, a foil, or a tube.

Seeds contained in a containers may be treated or untreated seeds. For example, the seeds can be treated to improve germination, for example, by priming the seeds, or by disinfection to protect against seed-borne pathogens. In another example, seeds can be coated with any available coating to improve, for example, plantability, seed emergence, and protection against seed-borne pathogens. Seed coating can be any form of seed coating including, but not limited to pelleting, film coating, and encrustments.

At least 10% of seeds within a container may be seeds of the invention. For example, at least 10%, 20%, 30%, 40%, 50%, 60%, 70%, 80%, 90%, 95%, 99% or 100% of the seeds in the container may be seeds of the invention.

The seeds of the invention may be hybrid seeds produced by a method including crossing a first plant according to the invention, with a second plant; and obtaining seeds. For example, crossing a plant including modified PPO enzyme, or a polynucleotide or expression vector encoding such a modified PPO enzyme, which provides increased resistance to a compound which inhibits PPO enzymatic activity with another plant.

The term “hybrid seed” refers to a seed produced by cross-pollinating two plants. Plants grown from hybrid seeds may have improved agricultural characteristics, such as better yield, greater uniformity, and/or disease resistance. Hybrid seeds do not breed true, i.e., the seed produced by self-fertilizing a hybrid plant (the plant grown from a hybrid seed) does not reliably result the next generation in an identical hybrid plant. Therefore, new hybrid seeds must be produced from the parent plant lines for each planting. Since most crop plants have both male and female organs, hybrid seeds can only be produced by preventing self-pollination of the female parent and allowing or facilitating pollination with the desired pollen. There are a variety of methods to prevent self-pollination of the female parent, one method by which self-pollination is prevented is mechanical removal of the pollen producing organ before pollen shed. Commercial hybrid maize seed (maize, Zea mays) production typically involves planting the desired male and female parental lines, usually in separate rows or blocks in an isolated field, treating the female parent plant to prevent pollen shed, ensuring pollination of the female by only the designated male parent, and harvesting hybrid seed from only the female parent. Hybrid seeds may be the result of a single cross (e.g., a first generation cross between two inbred lines), a modified single cross (e.g., a first generation cross between two inbred lines, one or other of which may have been modified slightly by the use of closely related crossing), a double cross (e.g., a first generation of a cross between two single crosses), a three-way cross (e.g., a first generation of a cross between a single cross and an inbred line), a top cross (e.g., the first generation of a cross between an inbred line and an open-pollinated variety, or the first generation of a cross between a single-cross and an open-pollinated variety), or an open pollinated variety (e.g., a population of plants selected to a standard which may show variation but has characteristics by which a variety can be differentiated from other varieties).

As used herein, the terms “cross” or “crossed” refer to the fusion of gametes via pollination to produce progeny (e.g., cells, seeds or plants). The term encompasses both sexual crosses (the pollination of one plant by another) and selfing (self-pollination, e.g., when the pollen and ovule are from the same plant). The term “crossing” refers to the act of fusing gametes via pollination to produce progeny.

### Nucleic Acids

The invention further provides isolated polynucleotides that encode a modified PPO enzyme or fragment thereof as defined in any aspect or embodiment herein, suitably which may encode a modified PPO enzyme or fragment thereof as defined in any of the sequences listed herein, suitably which may encode any of SEQ ID NOs: 1, 2, 4-151, 153-302, or 305-336. Suitably which may encode any of SEQ ID NOs: 37-39, 58, 59, 97-99, 118, 119, 125-137, 139-151, 188-190, 209, 210, 248-250, 269, 270, 277-288 or 291-302. As such, also provided are modified PPO enzymes or functional fragments thereof that may be expressed from such isolated polynucleotides.

An “isolated” polynucleotide is substantially separated away from other polynucleotide sequences with which the polynucleotide is normally associated, such as, from the chromosomal or extrachromosomal DNA of a cell in which the polynucleotide naturally occurs. A polynucleotide may be an isolated polynucleotide when it comprises a transgene or part of a transgene present in the genome of another organism. The term also embraces polynucleotides that are biochemically purified so as to substantially remove contaminating polynucleotides and other cellular components. Isolated polynucleotides are substantially free of sequences (such as protein encoding sequences) that naturally flank the nucleic acid (i.e., sequences located at the 5′ and 3′ ends of the polynucleotide) in the genomic DNA of the organism from which the polynucleotide is derived. For example, the isolated polynucleotide can contain less than about 5 kb, 4 kb, 3 kb, 2 kb, 1 kb, 0.5 kb, or 0.1 kb of nucleotide sequences that naturally flank the polynucleotide in genomic DNA of the cell from which the polynucleotide is derived. The isolated polynucleotide may be flanked by its native genomic sequences that control its expression in the cell, for example, the native promoter, or native 3′ untranslated region.

### Expression Constructs and Vectors

The expression constructs and vectors, which include at least one polynucleotide of the present invention inserted therein may be any construct or vector capable of delivering the polynucleotide into a host or host cell and allowing expression of the polynucleotide to provide a functional modified PPO enzyme as described herein or fragment thereof.

Such constructs or vectors may contain heterologous polynucleotide sequences, that is polynucleotide sequences that are not naturally found adjacent to polynucleotides of the present invention and that may be derived from a species other than the species from which the polynucleotide molecule(s) are derived. The construct or vector can be either RNA or DNA, either prokaryotic or eukaryotic, and typically the vector is a virus or a plasmid.

A number of vectors suitable for stable transfection of plant cells or for the establishment of transgenic plants have been described in, e.g., Pouwels et al., Cloning Vectors: A Laboratory Manual, 1985, supp. 1987; Weissbach and Weissbach, Methods for Plant Molecular Biology, Academic Press, 1989; and Gelvin et al., Plant Molecular Biology Manual, Kluwer Academic Publishers, 1990. Typically, plant expression vectors include, for example, one or more cloned plant genes under the transcriptional control of 5′ and 3′ regulatory sequences and a dominant selectable marker. For example, the vector may be pBIN 19 (Bevan, Nucl. Acids Res. (1984)).

The expression vector of the invention may include one or more regulatory sequences. For example, the expression vectors can contain a promoter regulatory region (e.g., a regulatory region controlling inducible or constitutive, environmentally- or developmentally-regulated, or cell- or tissue-specific expression), a transcription initiation start site, a ribosome binding site, an RNA processing signal, a transcription termination site, and/or a polyadenylation signal. Such a portion of an expression vector may be referred to as an expression construct The expression construct may include one or more regulatory sequences that are functional in plants. Thus allowing expression of the polynucleotide encoding a modified PPO enzyme of the invention in a plant.

“Expression construct” as used herein means a nucleic acid sequence capable of directing expression of a particular nucleic acid sequence in an appropriate host cell, comprising a promoter operably linked to the polynucleotide of interest which is operably linked to termination signal sequences. It also typically comprises sequences required for proper translation of the polynucleotide sequence. The expression construct comprising the polynucleotide of interest may be chimeric, meaning that at least one of its components is heterologous with respect to at least one of its other components. The expression construct may also be one that is naturally occurring but has been obtained in a recombinant form useful for heterologous expression. Typically, however, the expression construct is heterologous with respect to the host, i.e., the particular polynucleotide of the expression cassette does not occur naturally in the host cell and must have been introduced into the host cell or an ancestor of the host cell by a transformation event. The expression of the polynucleotide sequence in the expression construct may be under the control of, for example, a constitutive promoter or of an inducible promoter that initiates transcription only when the host cell is exposed to some particular external stimulus. In the case of a multicellular organism, such as a plant, the promoter can also be specific to a particular tissue, or organ, or stage of development.

The term “regulatory element” or “regulatory sequence” as used herein refers to a nucleic acid that is capable of regulating the transcription and/or translation of an operably linked polynucleotide. Regulatory elements include, but are not limited to, promoters, enhancers, introns, 5′ UTRs, and 3′ UTRs.

Expression cassettes may include in the 5′-3′ direction of transcription, a transcriptional and translational initiation region (e.g., a promoter), a polynucleotide sequence encoding modified PPO enzyme of the invention, and a transcriptional and translational termination region (e.g., termination region) functional in plants.

Any promoter can be used in the production of the expression construct and vectors including such expression constructs as described herein. The promoter may be native or analogous, or foreign or heterologous, to the plant host and/or to the polynucleotide sequences encoding the modified PPO of the invention. Additionally, the promoter may be a natural sequence or alternatively a synthetic sequence. Where the promoter is “foreign” or “heterologous” to the plant host, it is intended that the promoter is not found in the native plant into which the promoter is introduced. Where the promoter is “foreign” or “heterologous” to the polynucleotide encoding the modified PPO of the invention, it is intended that the promoter is not the native or naturally occurring promoter for the operably linked polynucleotide of the invention.

While it may be preferable to express the polynucleotide encoding a modified PPO of the invention using heterologous promoters, the native promoter sequences may be used in the preparation of the expression constructs. Such expression constructs may change expression levels of the modified PPO enzyme in the plant or plant cell. Thus, the phenotype of the plant or plant cell is altered.

Any promoter can be used in the preparation of expression constructs to control the expression of the polynucleotide encoding modified PPO, such as promoters providing for constitutive, tissue-preferred, inducible, or other promoters for expression in plants.

Constitutive promoters include, for example, the core promoter of the Rsyn7 promoter and other constitutive promoters disclosed in WO 99/43 838 and U.S. Pat. No. 6,072,050; the core CaMV 35S promoter (Odell et al. (1985) Nature 313:810-812); rice actin (McElroy et al. (1990) Plant Cell 2:163-171); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet. 81:581-588); MAS (Velten et al. (1984) EMBO J. 3:2723-2730); ALS promoter (U.S. Pat. No. 5,659,026), and the like. Other constitutive promoters include, for example, U.S. Pat. Nos. 5,608,149; 5,608,144; 5,604,121; 5,569,597; 5,466,785; 5,399,680; 5,268,463; 5,608,142; and 6,177,611.

Tissue-preferred promoters can be utilized to direct expression of the modified PPO enzymes of the invention within a particular plant tissue. Such tissue-preferred promoters include, but are not limited to, leaf-preferred promoters, root-preferred promoters, seed-preferred promoters, and stem-preferred promoters. Tissue-preferred promoters include those described in Yamamoto et al. (1997) Plant J. 12(2):255-265; Kawamata et al. (1997) Plant Cell Physiol. 38(7):792-803; Hansen et al. (1997) Mol Gen Genet. 254(3):337-343; Russell et al. (1997) Transgenic Res. 6(2): 157-168; Rinehart et al. (1996) Plant Physiol. 1 12(3):1331-1341; Van Camp et al. (1996) Plant Physiol. 1 12(2):525-535; Canevascini et al. (1996) Plant Physiol. 112(2): 513-524; Yamamoto et al. (1994) Plant Cell Physiol. 35(5):773-778; Lam (1994) Results Probl. Cell Differ. 20:181-196; Orozco et al (1993) Plant Mol Biol. 23(6):1129-1138; Matsuoka et al. (1993) Proc Natl. Acad. Sci. USA 90(20):9586-9590; and Guevara-Garcia et al. (1993) Plant J. 4(3):495-505.

The expression constructs may also comprise transcription termination regions. Where transcription terminations regions are used, any termination region may be used in the preparation of the expression cassettes. For example, the termination region may be native to the transcriptional initiation region, may be native to the operably linked polynucleotide of interest, may be native to the plant host, or may be derived from another source (i.e., foreign or heterologous to the promoter, the polynucleotide of interest encoding modified PPO enzyme, the plant host, or any combination thereof). Examples of termination regions that are available for use in the expression constructs and vectors of the present invention include those from the Ti-plasmid of A. tumefaciens, such as the octopine synthase and nopaline synthase termination regions. See also Guerineau et al. (1991) Mol. Gen. Genet. 262: 141-144; Sanfacon et al. (1991) Genes Dev. 5:141-149; Mogen et al. (1990) Plant Cell 2:1261-1272; Munroe et al. (1990) Gene 91:151-158; Ballas et al. (1989) Nucleic Acids Res. 17:7891-7903; and Joshi et al. (1987) Nucleic Acid Res. 15:9627-9639.

For example, the expression construct may comprise a Tomato Mosaic Virus (TMV) omega 5′ leader and a modified PPO enzyme encoding gene of interest is excised using XhoI/KpnI and cloned into pBIN 19 behind a double enhanced 35S promoter and ahead of a NOS 3′ transcription terminator. A suitable exemplary vector comprising such an expression construct is provided herein as SEQ ID NO: 3.

The polynucleotides may be optimized for increased expression in a transformed plant. That is, the polynucleotides encoding the modified PPO enzymes can be synthesized using plant-preferred codons for improved expression. See, for example, Campbell and Gowri (1990) Plant Physiol. 92:1-11 for a discussion of host-preferred codon usage. Methods are available in the art for synthesizing plant-preferred genes. See, for example, U.S. Pat. Nos. 5,380,831, and 5,436,391, and Murray et al. (1989) Nucleic Acids Res. 17:477-498, herein incorporated by reference.

In addition, other sequence modifications can be made to the polynucleotides of the invention. For example, additional sequence modifications that are known to enhance gene expression in a cellular host. These include elimination of sequences encoding spurious polyadenylation signals, exon/intron splice site signals, transposon-like repeats, and other such well-characterized sequences that may be deleterious to gene expression. The G-C content of the sequence may also be adjusted to levels average for a target cellular host, as calculated by reference to known genes expressed in the host cell. In addition, the sequence can be modified to avoid predicted hairpin secondary mRNA structures.

Other polynucleotide sequences may also be used in the preparation of the expression constructs of the present invention, for example to enhance the expression of the modified PPO encoding polynucleotide sequence. Such polynucleotide sequences include the introns of the maize Adhl, intron I gene (Callis et al. (1987) Genes and Development 1:1183-1200), and leader sequences, (W-sequence) from the Tobacco Mosaic virus (TMV), Maize Chlorotic Mottle Virus and Alfalfa Mosaic Virus (Gallie et al (1987) Nucleic Acid Res. 15:8693-8711, and Skuzeski et al. (1990) Plant Mol. Biol. 15:65-79, 1990). The first intron from the shrunken-1 locus of maize has been shown to increase expression of genes in chimeric gene constructs. U.S. Pat. Nos. 5,424,412 and 5,593,874 disclose the use of specific introns in gene expression constructs, and Gallie et al. ((1994) Plant Physiol. 106:929-939) also have shown that introns are useful for regulating gene expression on a tissue specific basis. Plant cells transformed with such modified expression constructs or vectors, then, may exhibit overexpression or constitutive expression of a polynucleotide of the invention.

Expression constructs may additionally contain 5′ leader sequences. Such leader sequences can act to enhance translation. Translation leaders are known in the art and include: picornavirus leaders, for example, EMCV leader (Encephalomyocarditis 5′ noncoding region) (Elroy-Stein et al. (1989) Proc. Natl. Acad. ScL USA 86:6126-6130); potyvirus leaders, for example, TEV leader (Tobacco Etch Virus) (Gallie et al. (1995) Gene 165(2):233-238), MDMV leader (Maize Dwarf Mosaic Virus) (Virology 154:9-20), and human immunoglobulin heavy-chain binding protein (BiP) (Macejak et al. (1991) Nature 353: 90-94); untranslated leader from the coat protein mRNA of alfalfa mosaic virus (AMV RNA 4) (Jobling et al. (1987) Nature 325: 622-625); tobacco mosaic virus leader (TMV) (Gallie et al. (1989) in Molecular Biology of RNA, ed. Cech (Liss, New York), pp. 237-256); and maize chlorotic mottle virus leader (MCMV) (Lommel et al. (1991) Virology 81:382-385). See also, Della-Cioppa et al. (1987) Plant Physiol. 84:965-968.

In preparing the expression constructs and expression vectors described herein, the various polynucleotides may be manipulated, so as to provide for the polynucleotides in the proper orientation and, as appropriate, in the proper reading frame. Toward this end, adapters or linkers may be employed to join the nucleic acid molecules or other manipulations may be involved to provide for convenient restriction sites, removal of superfluous polynucleotides, removal of restriction sites, or the like. For this purpose, in vitro mutagenesis, primer repair, restriction, annealing, resubstitutions, e.g., transitions and transversions, may be involved. Expression vectors may include additional features. For example, gRNA promoters to regulate expression of the at least one gRNA, e.g. prOsU3-01, which is the Rice U3 promoter for pol III dependent transcription of non-coding. Vectors may similarly include additional features such as selectable markers, e.g. Phosphomannose Isomerase (PMI), and antibiotic resistance genes that can be used to aid recovery of stably transformed plants.

By “operably linked” or “operably associated” as used herein, it is meant that the indicated elements are functionally related to each other, and are also generally physically related.

Thus, the term “operably linked” or “operably associated” as used herein, refers to polynucleotides on a single nucleic acid molecule that are functionally associated. Thus, a first polynucleotide sequence or nucleic acid molecule that is operably linked to a second polynucleotide sequence or nucleic acid molecule, means a situation when the first polynucleotide sequence or nucleic acid molecule is placed in a functional relationship with the second polynucleotide sequence or nucleic acid molecule. For instance, a promoter is operably associated with a polynucleotide sequence or nucleic acid molecule if the promoter effects the transcription or expression of said polynucleotide sequence or nucleic acid molecule. Those skilled in the art will appreciate that the control sequences (e.g., promoter) need not be contiguous with the polynucleotide sequence or nucleic acid molecule to which it is operably associated, as long as the control sequences function to direct the expression thereof. Thus, for example, intervening untranslated, yet transcribed, sequences can be present between a promoter and a polynucleotide sequence or nucleic acid molecule, and the promoter can still be considered “operably linked” to or “operatively associated” with the polynucleotide sequence or nucleic acid molecule.

### Herbicide Resistance and Methods of Use

The plants or parts thereof according to the invention are at least partially resistant to inhibition by a compound which inhibits a PPO enzyme (i.e. PPO enzymatic activity). The present invention further relates to a method of controlling the growth of undesired vegetation at a locus comprising plants having the modified PPO enzymes provided herein in the vicinity thereof, wherein the method comprises applying an effective amount of at least one PPO-inhibiting herbicide.

The term “locus” includes soil, seeds, seedlings, field, greenhouse, an area of cultivation, as well as, established vegetation.

Suitably the compound which inhibits a PPO enzyme is a herbicide. Suitably therefore the plants of the invention are resistant to a herbicide which inhibits PPO enzymatic activity (referred to herein as a PPO-inhibiting herbicide), and therefore such plants can be used in methods where these herbicides are applied. Herbicides can be applied pre-emergence or post-emergence of the crop plant or the undesired vegetation.

A plant having increased resistance to a compound which inhibits PPO enzymatic activity (a PPO-inhibiting herbicide) may be referred to as an “herbicide-tolerant” or “herbicide-resistant” plant. Such plants are tolerant or at least partially resistant to at least one compound which inhibits PPO enzymatic activity at a level that would normally kill, or inhibit the growth of a normal, control or wild-type plant lacking the modified PPO enzymes, polynucleotides encoding said enzymes or expression vectors of the invention. The term “herbicide” is used herein to mean an active ingredient that kills, controls or otherwise adversely modifies the growth of plants and is typically used to control undesired vegetation.

For example, plants of the invention may have at least a 2-fold increase in resistance to a compound which inhibits PPO enzymatic activity, such as the inhibiting herbicides described herein. For example, plants of the invention may have at least a 2-fold, 3-fold, 4-fold, 5-fold 6-fold, 7-fold, 8-fold, 9-fold, 10-fold increase in resistance. In one embodiment, the plants of the invention comprising at least one of the modified PPO enzymes disclosed herein may have at least a 2-fold increase in resistance to any of compounds A to E described herein, compared to an unmodified plant. In one embodiment, the plants of the invention comprising at least one of the modified PPO enzymes disclosed herein may have at least a 2-fold, 3-fold, 4-fold, 5-fold 6-fold, 7-fold, 8-fold, 9-fold, 10-fold increase in resistance to any of compounds A to E described herein, compared to an unmodified plant. In one embodiment, the plants of the invention comprising at least one of the modified PPO enzymes disclosed herein may have at least a 2-fold increase in resistance to any of compounds A to E described herein, compared to a control plant. In one embodiment, the plants of the invention comprising at least one of the modified PPO enzymes disclosed herein may have at least a 2-fold, 3-fold, 4-fold, 5-fold 6-fold, 7-fold, 8-fold, 9-fold, 10-fold increase in resistance to any of compounds A to E described herein, compared to a control plant. For example, a control plant may be a plant comprising an alternative modified PPO enzyme to those of the invention. For example, a plant comprising a modified PPO having a sequence as set forth in SEQ ID NO: 124.

Resistance to compounds which inhibit PPO enzymatic activity may be determined by any known methods for comparing the growth, damage or other properties of two plants after application of a compound which inhibits PPO enzymatic activity to a plant. For example, the resistance of a plant of the invention may be determined by comparing the percentage of damage caused to the plant in comparison to a wild-type or control plant after application of a compound which inhibits PPO enzymatic activity, such as the herbicides described herein. Increased resistance to a compound which inhibits PPO enzymatic activity (a PPO-inhibiting herbicide) refers in the context of the invention to a plant which has been modified to comprise a modified PPO enzyme that has improved or increased resistance to a compound which inhibits PPO enzymatic activity (the PPO-inhibiting herbicide), relative to an unmodified plant (e.g. wild type) or control plant (e.g. a plant comprising a PPO enzyme according to SEQ ID NO: 124). Suitably the increased resistance may be due to an increased activity of the modified PPO enzyme in the plant, compared to the activity of such enzymes in an unmodified plant, when in the presence of at least one compound that is known to interfere with PPO enzyme activity in plants at a concentration or level that is to known to inhibit the activity of the wild-type PPO protein. Improved resistance means that the plant comprising the enzyme, and the modified PPO enzyme itself, is at least partially resistant to a compound which inhibits PPO enzymatic activity (the PPO-inhibiting herbicide).

Partially resistant plants of the invention may still have some decreased PPO enzymatic activity when exposed to a compound which inhibits PPO enzymatic activity, such as at most a 1%, 2%, 3%, 4%, 5%, 6%, 7%, 8%, 9%, 10%, 15%, 20%, 25%, 30%, 35%, 40%, 45%, 50%, decrease in enzymatic activity. Suitably therefore the modified PPO enzymes used in the invention may be partially resistant, and may still have some decrease in enzymatic activity when exposed to a compound which inhibits PPO enzymatic activity. In some embodiments, the plants modified to comprise the modified PPO enzymes may have total or near total resistance to a compound which inhibits PPO enzymatic activity (the PPO-inhibiting herbicide). Suitably the plants may have 90%, 91%, 92%, 93%, 94%, 95%, 96%, 97%, 98%, 99% or 100% resistance to a compound which inhibits PPO enzymatic activity.

Suitably therefore the modified PPO enzymes used in the invention may have no substantial decrease in enzymatic activity when exposed to a compound which inhibits PPO enzymatic activity.

However, any decrease in activity is less than a decrease in activity relative to the activity of a wild-type enzyme, when in the presence of at least one compound that is known to interfere with PPO enzymatic activity and at a concentration or level of the compound that is to known to inhibit the activity of the wild-type PPO protein. A decrease in activity seen for a partially resistant modified PPO enzyme may be a decrease in activity that does not have a negative effect on the growth, propagation or development of a plant comprising a partially resistant modified PPO enzyme. Furthermore, the activity of such a partially resistant modified PPO enzyme may be referred to herein as “herbicide-tolerant” or “herbicide-resistant” enzymes. Plants which are at least partially “resistant” to at least one compound that inhibits PPO enzymatic activity exhibit few, if any, necrotic, lytic, chlorotic or other lesions when subjected to the compound at concentrations and rates which are typically employed by the agricultural community to kill unwanted vegetation in the vicinity of the plant such as a field.

The modified PPO enzymes described herein may be compared to a reference, unmodified, or wild-type enzyme, which otherwise may be termed a control enzyme or a plant comprising such an enzyme. The term “wild-type” is used to refer to a nucleic acid molecule or protein that can be found in nature as distinct from being artificially produced or mutated by man. A reference or unmodified PPO enzyme may be a PPO enzyme derived from the same source species as the modified enzyme, that does not include any modifications of the invention as described herein. In some embodiments, the reference enzyme may be a wild type enzyme. However, the use of the term “unmodified” or “reference” is not intended to necessarily imply that a plant, plant tissue, plant cell, or other host cell lacks any recombinant DNA in its genome, and/or does not possess herbicide resistant characteristics that are different from those disclosed herein. Reference, or unmodified PPO enzymes as referred to herein may well include other mutations or modifications that do not affect resistance to compounds which inhibit PPO enzymatic activity. For example, a reference enzyme may include mutations or modifications to improve or alter expression, translation or targeting of the control enzyme to specific tissues, organs or cells.

In some examples, the reference to control enzyme (or plant including said enzyme) may be a PPO enzyme that includes alternative modifications that increase resistance to compounds that inhibit PPO enzymatic activity to those of the invention.

A wild-type, reference unmodified PPO enzyme may be an enzyme encoded by SEQ ID NO: 1. A wild-type, reference unmodified PPO enzyme may be a PPO enzyme encoded by SEQ ID NO: 2. A reference or control PPO enzyme may be an enzyme encoded by SEQ ID NO: 124.

Many herbicides which inhibit PPO enzymatic activity are known in the art and include a structurally diverse range of chemistry. Accordingly, the skilled person will appreciate that a broad range of PPO inhibiting herbicides have utility in the present invention.

In one embodiment, the PPO-inhibiting herbicide is selected from the group consisting of butafenacil, carfentrazone-ethyl, cyclopyranil, epyrifenacil (Herbicide A), flufenoximacil, flumioxazin, fomesafen, oxyfluorfen, pyraflufen-ethyl, saflufenacil (Herbicide E), sulfentrazone, tiafenacil, trifludimoxazin (Herbicide B), compounds disclosed in WO2016/095768 for example 3-(2-chloro-4-fluoro-5-(3-methyl-2,6-dioxo-4-trifluoromethyl-3,6-dihydropyrimidin-1(2H)-yl)phenyl)-5-methyl-4,5-dihydroisoxazole-5-carboxylic acid ethyl ester (Herbicide C), compounds described in WO2017/202768 for example ethyl 2-[[3-[[3-chloro-5-fluoro-6-[3-methyl-2,6-dioxo-4-(trifluoromethyl)pyrimidin-1-yl]-2-pyridyl]oxy]acetate, compounds disclosed in WO2020/239607 for example ethyl (5S)-3-[2-chloro-5-(3,5-dimethyl-2,6-dioxo-4-thioxo-1,3,5-triazinan-1-yl)-4-fluoro-phenyl]-5-methyl-4H-isoxazole-5-carboxylate (Herbicide D), compounds disclosed in WO2021/013799 for example pyridin-2-ylmethyl-[(3-(2-chloro-4-fluoro-5-[3-methyl-2,6-dioxo-4-(trifluoromethyl)-3,6-dihydropyrimidin-1(2H)-yl]phenoxy)pyridine-2-yl)oxy]acetate, 2-Methoxyethyl-[(3-(2-chloro-4-fluoro-5-[3-methyl-2,6-dioxo-4-(trifluoromethyl)-3,6-dihydropyrimidin-1(2H)-yl]phenoxy)pyridine-2-yl)oxy]acetate, 2-Methoxyethyl-[(3-(2-cyan-4-fluoro-5-[3-methyl-2,6-dioxo-4-(trifluoromethyl)-3,6-dihydro-pyrimidin-1(2H)-yl]phenoxy)pyridine-2-yl)oxy]acetate and cyanomethyl-[(3-(2-bromo-4-fluoro-5-[3-methyl-2,6-dioxo-4-(trifluoromethyl)-3,6-dihydropyrimidin-1(2H)-yl]phenoxy)pyridine-2-yl)oxy]acetate, compounds disclosed in WO2021/143677 for example methyl 3-[2-chloro-5-[3,6-dihydro-3-methyl-2,6-dioxo-4-(trifluoromethyl)-1(2H)-pyrimidinyl]-4-fluorophenyl]-3a,4,5,6-tetrahydro-6-methyl-6aH-cyclopent[d]isoxazole-6a-carboxylate, compounds disclosed in WO2021/175689 for example methyl 2-[2-[2-bromo-4-fluoro-5-[3-methyl-2,6-dioxo-4-(trifluoromethyl)pyrimidin-1-yl]phenoxy]phenoxy]-2-methoxy-acetate, compounds disclosed in WO2022/138633 for example (2-ethoxy-2-oxo-ethyl) 1-[2-chloro-4-fluoro-5-[3-methyl-2,6-dioxo-4-(trifluoromethyl)pyrimidin-1-yl]phenoxy]cyclopropanecarboxylate, and compounds disclosed in WO2023/044364 including, where applicable, enantiomers (including enantiomer enriched mixtures) of said compounds and, where applicable, agrochemically acceptable salts and/or esters of all of the previously mentioned compounds.

The compound which inhibits PPO enzymatic activity may be any combination of one or more such inhibitory compounds, suitably one or more of the compounds listed above. For example, 1, 2, 3, 4, 5 or more of the inhibitory compounds as described herein.

The plants of the invention, that include a modified PPO enzyme of the invention, may be used in methods of controlling undesired vegetation in the vicinity of the plant. The methods may include applying an effective amount of at least one compound, such as those listed above, which inhibits PPO enzymatic activity to the undesired vegetation and the plant.

In addition, plants of the invention, that include a modified PPO enzyme of the invention may be used in methods of enhancing plant growth by controlling undesired vegetation in the vicinity of the plant. The methods may include applying an effective amount at least one compound, such as those listed above, which inhibits PPO enzymatic activity to the undesired vegetation and the plant.

Suitably certain modified PPO enzymes may perform better when used with certain compounds which inhibit PPO enzymatic activity. Suitably certain modified PPO enzymes may provide better resistance to certain compounds which inhibit PPO enzymatic activity compared to other compounds which inhibit PPO enzymatic activity. Suitably therefore certain combinations of PPO modifications and compounds may be optimal for use in the methods of the invention.

In one embodiment of such methods, the plant of the invention may comprise a PPO enzyme having a mutation at position 362 of SEQ ID NO: 1 or a residue corresponding thereto and is for use with any PPO-inhibiting herbicide disclosed herein, including for example compound D. In one embodiment of such methods, the plant of the invention may comprise a PPO enzyme having a mutation at position 362 of SEQ ID NO: 1 or residue corresponding thereto and is for use with any PPO-inhibiting herbicide disclosed herein, including for example compound B. In one embodiment of such methods, the plant of the invention may comprise a PPO enzyme having a mutation at position 365 of SEQ ID NO: 1 or residue corresponding thereto and is for use with any PPO-inhibiting herbicide disclosed herein, including for example compound B. In one embodiment of such methods, the plant of the invention may comprise a PPO enzyme having a mutation at position 365 of SEQ ID NO: 1 or residue corresponding thereto and is for use with any PPO-inhibiting herbicide disclosed herein, including for example compound E. In one embodiment of such methods, the plant of the invention may comprise a PPO enzyme having a mutation at position 479 of SEQ ID NO: 1 or residue corresponding thereto and is for use with any PPO-inhibiting herbicide disclosed herein, including for example compound B. In one embodiment of such methods, the plant of the invention may comprise a PPO enzyme having a mutation at position 479 of SEQ ID NO: 1 or residue corresponding thereto and is for use with any PPO-inhibiting herbicide disclosed herein, including for example compound D. In one embodiment of such methods, the plant of the invention may comprise a PPO enzyme having a mutation at position 479 of SEQ ID NO: 1 or residue corresponding thereto and is for use with any PPO-inhibiting herbicide disclosed herein, including for example compound E.

In one embodiment of such methods, the plant of the invention may comprise a PPO enzyme having a mutation at positions 305 and 426 of SEQ ID NO: 1 or residues corresponding thereto wherein the mutation at position 305 is S305L and position 426 is Y426V or residues corresponding thereto (i.e. SEQ ID NO 125 or 139) and is for use with any PPO-inhibiting herbicide disclosed herein, including for example compound A. In one embodiment of such methods, the plant of the invention may comprise a PPO enzyme having a mutation at positions 305 and 426 of SEQ ID NO: 1 or residues corresponding thereto wherein the mutation at position 305 is S305L and position 426 is Y426V or residues corresponding thereto and is for use with any PPO-inhibiting herbicide disclosed herein, including for example compound C. In one embodiment of such methods, the plant of the invention may comprise a PPO enzyme having a mutation at positions 305 and 426 of SEQ ID NO: 1 or residues corresponding thereto wherein the mutation at position 305 is S305L and position 426 is Y426V or residues corresponding thereto and is for use with any PPO-inhibiting herbicide disclosed herein, including for example compound D. In one embodiment of such methods, the plant of the invention may comprise a PPO enzyme having a mutation at positions 305 and 426 of SEQ ID NO: 1 or residues corresponding thereto wherein the mutation at position 305 is S305L and position 426 is Y426V or residues corresponding thereto and is for use with any PPO-inhibiting herbicide disclosed herein, including for example compound E.

In one embodiment of such methods, the plant of the invention may comprise a PPO enzyme having a mutation at positions 365, 426 and 479 of SEQ ID NO: 1 or residues corresponding thereto (e.g. SEQ ID NO 126) and is for use with any PPO-inhibiting herbicide disclosed herein, including for example compound A. In one embodiment of such methods, the plant of the invention may comprise a PPO enzyme having a mutation at positions 365, 426 and 479 of SEQ ID NO: 1 or residues corresponding thereto and is for use with any PPO-inhibiting herbicide disclosed herein, including for example compound C. In one embodiment of such methods, the plant of the invention may comprise a PPO enzyme having a mutation at positions 365, 426 and 479 of SEQ ID NO: 1 or residues corresponding thereto and is for use with any PPO-inhibiting herbicide disclosed herein, including for example compound D. In one embodiment of such methods, the plant of the invention may comprise a PPO enzyme having a mutation at positions 365, 426 and 479 of SEQ ID NO: 1 or residues corresponding thereto and is for use with any PPO-inhibiting herbicide disclosed herein, including for example compound E.

In one embodiment of such methods, the plant of the invention may comprise a PPO enzyme having a mutation at positions 305, 404, 426, 431, and 479 of SEQ ID NO: 1 or residues corresponding thereto (e.g. SEQ ID NO 127) and is for use with any PPO-inhibiting herbicide disclosed herein, including for example compound A. In one embodiment of such methods, the plant of the invention may comprise a PPO enzyme having a mutation at positions 305, 404, 426, 431, and 479 of SEQ ID NO: 1 or residues corresponding thereto and is for use with any PPO-inhibiting herbicide disclosed herein, including for example compound C. In one embodiment of such methods, the plant of the invention may comprise a PPO enzyme having a mutation at positions 305, 404, 426, 431, and 479 of SEQ ID NO: 1 or residues corresponding thereto and is for use with any PPO-inhibiting herbicide disclosed herein, including for example compound D. In one embodiment of such methods, the plant of the invention may comprise a PPO enzyme having a mutation at positions 305, 404, 426, 431, and 479 of SEQ ID NO: 1 or residues corresponding thereto and is for use with any PPO-inhibiting herbicide disclosed herein, including for example compound E.

In one embodiment of such methods, the plant of the invention may comprise a PPO enzyme having a mutation at positions 305 and 365 of SEQ ID NO: 1 or residues corresponding thereto (e.g. SEQ ID NO 128) and is for use with any PPO-inhibiting herbicide disclosed herein, including for example compound C. In one embodiment of such methods, the plant of the invention may comprise a PPO enzyme having a mutation at positions 305 and 365 of SEQ ID NO: 1 or residues corresponding thereto and is for use with any PPO-inhibiting herbicide disclosed herein, including for example compound D. In one embodiment of such methods, the plant of the invention may comprise a PPO enzyme having a mutation at positions 305 and 365 of SEQ ID NO: 1 or residues corresponding thereto and is for use with any PPO-inhibiting herbicide disclosed herein, including for example compound E.

In one embodiment of such methods, the plant of the invention may comprise a PPO enzyme having a mutation at positions 305, 362 and 404 of SEQ ID NO: 1 or residues corresponding thereto (e.g. SEQ ID NO 129) and is for use with any PPO-inhibiting herbicide disclosed herein, including for example compound A. In one embodiment of such methods, the plant of the invention may comprise a PPO enzyme having a mutation at positions 305, 362 and 404 of SEQ ID NO: 1 or residues corresponding thereto and is for use with any PPO-inhibiting herbicide disclosed herein, including for example compound B. In one embodiment of such methods, the plant of the invention may comprise a PPO enzyme having a mutation at positions 305, 362 and 404 of SEQ ID NO: 1 or residues corresponding thereto and is for use with any PPO-inhibiting herbicide disclosed herein, including for example compound C. In one embodiment of such methods, the plant of the invention may comprise a PPO enzyme having a mutation at positions 305, 362 and 404 of SEQ ID NO: 1 or residues corresponding thereto and is for use with any PPO-inhibiting herbicide disclosed herein, including for example compound D. In one embodiment of such methods, the plant of the invention may comprise a PPO enzyme having a mutation at positions 305, 362 and 404 of SEQ ID NO: 1 or residues corresponding thereto and is for use with any PPO-inhibiting herbicide disclosed herein, including for example compound E.

In one embodiment of such methods, the plant of the invention may comprise a PPO enzyme having a mutation at positions 305, 361, 365, 431 and 479 of SEQ ID NO: 1 or residues corresponding thereto (e.g. SEQ ID NO 130) and is for use with any PPO-inhibiting herbicide disclosed herein, including for example compound A. In one embodiment of such methods, the plant of the invention may comprise a PPO enzyme having a mutation at positions 305, 361, 365, 431 and 479 of SEQ ID NO: 1 or residues corresponding thereto and is for use with any PPO-inhibiting herbicide disclosed herein, including for example compound B. In one embodiment of such methods, the plant of the invention may comprise a PPO enzyme having a mutation at positions 305, 361, 365, 431 and 479 of SEQ ID NO: 1 or residues corresponding thereto and is for use with any PPO-inhibiting herbicide disclosed herein, including for example compound D. In one embodiment of such methods, the plant of the invention may comprise a PPO enzyme having a mutation at positions 305, 361, 365, 431 and 479 of SEQ ID NO: 1 or residues corresponding thereto and is for use with any PPO-inhibiting herbicide disclosed herein, including for example compound E.

In one embodiment of such methods, the plant of the invention may comprise a PPO enzyme having a mutation at positions 305, 426, and 479 of SEQ ID NO: 1 or residues corresponding thereto (e.g. SEQ ID NO 131) and is for use with any PPO-inhibiting herbicide disclosed herein, including for example compound A. In one embodiment of such methods, the plant of the invention may comprise a PPO enzyme having a mutation at positions 305, 426, and 479 of SEQ ID NO: 1 or residues corresponding thereto and is for use with any PPO-inhibiting herbicide disclosed herein, including for example compound B. In one embodiment of such methods, the plant of the invention may comprise a PPO enzyme having a mutation at positions 305, 426, and 479 of SEQ ID NO: 1 or residues corresponding thereto and is for use with any PPO-inhibiting herbicide disclosed herein, including for example compound C. In one embodiment of such methods, the plant of the invention may comprise a PPO enzyme having a mutation at positions 305, 426, and 479 of SEQ ID NO: 1 or residues corresponding thereto and is for use with any PPO-inhibiting herbicide disclosed herein, including for example compound D. In one embodiment of such methods, the plant of the invention may comprise a PPO enzyme having a mutation at positions 305, 426, and 479 of SEQ ID NO: 1 or residues corresponding thereto and is for use with any PPO-inhibiting herbicide disclosed herein, including for example compound E.

In one embodiment of such methods, the plant of the invention may comprise a PPO enzyme having a mutation at positions 426, 461 and 479 of SEQ ID NO: 1 or residues corresponding thereto (e.g. SEQ ID NO 132) and is for use with any PPO-inhibiting herbicide disclosed herein, including for example compound A. In one embodiment of such methods, the plant of the invention may comprise a PPO enzyme having a mutation at positions 426, 461 and 479 of SEQ ID NO: 1 or residues corresponding thereto and is for use with any PPO-inhibiting herbicide disclosed herein, including for example compound B. In one embodiment of such methods, the plant of the invention may comprise a PPO enzyme having a mutation at positions 426, 461 and 479 of SEQ ID NO: 1 or residues corresponding thereto and is for use with any PPO-inhibiting herbicide disclosed herein, including for example compound C. In one embodiment of such methods, the plant of the invention may comprise a PPO enzyme having a mutation at positions 426, 461 and 479 of SEQ ID NO: 1 or residues corresponding thereto and is for use with any PPO-inhibiting herbicide disclosed herein, including for example compound E.

In one embodiment of such methods, the plant of the invention may comprise a PPO enzyme having a mutation at positions 305, 361, and 365 of SEQ ID NO: 1 or residues corresponding thereto (e.g. SEQ ID NO 133) and is for use with any PPO-inhibiting herbicide disclosed herein, including for example compound A. In one embodiment of such methods, the plant of the invention may comprise a PPO enzyme having a mutation at positions 305, 361, and 365 of SEQ ID NO: 1 or residues corresponding thereto and is for use with any PPO-inhibiting herbicide disclosed herein, including for example compound B. In one embodiment of such methods, the plant of the invention may comprise a PPO enzyme having a mutation at positions 305, 361, and 365 of SEQ ID NO: 1 or residues corresponding thereto and is for use with any PPO-inhibiting herbicide disclosed herein, including for example compound C. In one embodiment of such methods, the plant of the invention may comprise a PPO enzyme having a mutation at positions 305, 361, and 365 of SEQ ID NO: 1 or residues corresponding thereto and is for use with any PPO-inhibiting herbicide disclosed herein, including for example compound D. In one embodiment of such methods, the plant of the invention may comprise a PPO enzyme having a mutation at positions 305, 361, and 365 of SEQ ID NO: 1 or residues corresponding thereto and is for use with any PPO-inhibiting herbicide disclosed herein, including for example compound E.

In one embodiment of such methods, the plant of the invention may comprise a PPO enzyme having a mutation at positions 305, 361, 362, 426 and 479 of SEQ ID NO: 1 or residues corresponding thereto (e.g. SEQ ID NO 134) and is for use with any PPO-inhibiting herbicide disclosed herein, including for example compound A. In one embodiment of such methods, the plant of the invention may comprise a PPO enzyme having a mutation at positions 305, 361, 362, 426 and 479 of SEQ ID NO: 1 or residues corresponding thereto and is for use with any PPO-inhibiting herbicide disclosed herein, including for example compound C. In one embodiment of such methods, the plant of the invention may comprise a PPO enzyme having a mutation at positions 305, 361, 362, 426 and 479 of SEQ ID NO: 1 or residues corresponding thereto and is for use with any PPO-inhibiting herbicide disclosed herein, including for example compound D. In one embodiment of such methods, the plant of the invention may comprise a PPO enzyme having a mutation at positions 305, 361, 362, 426 and 479 of SEQ ID NO: 1 or residues corresponding thereto and is for use with any PPO-inhibiting herbicide disclosed herein, including for example compound E.

In one embodiment of such methods, the plant of the invention may comprise a PPO enzyme having a mutation at positions 361 and 365 of SEQ ID NO: 1 or residues corresponding thereto (e.g. SEQ ID NO 135) and is for use with any PPO-inhibiting herbicide disclosed herein, including for example compound C. In one embodiment of such methods, the plant of the invention may comprise a PPO enzyme having a mutation at positions 361 and 365 of SEQ ID NO: 1 or residues corresponding thereto and is for use with any PPO-inhibiting herbicide disclosed herein, including for example compound B. In one embodiment of such methods, the plant of the invention may comprise a PPO enzyme having a mutation at positions 361 and 365 of SEQ ID NO: 1 or residues corresponding thereto and is for use with any PPO-inhibiting herbicide disclosed herein, including for example compound D. In one embodiment of such methods, the plant of the invention may comprise a PPO enzyme having a mutation at positions 361 and 365 of SEQ ID NO: 1 or residues corresponding thereto and is for use with any PPO-inhibiting herbicide disclosed herein, including for example compound E.

In one embodiment of such methods, the plant of the invention may comprise a PPO enzyme having a mutation at positions 365, 404 and 479 of SEQ ID NO: 1 or residues corresponding thereto (e.g. SEQ ID NO 136) and is for use with any PPO-inhibiting herbicide disclosed herein, including for example compound B. In one embodiment of such methods, the plant of the invention may comprise a PPO enzyme having a mutation at positions 365, 404 and 479 of SEQ ID NO: 1 or residues corresponding thereto and is for use with any PPO-inhibiting herbicide disclosed herein, including for example compound C. In one embodiment of such methods, the plant of the invention may comprise a PPO enzyme having a mutation at positions 365, 404 and 479 of SEQ ID NO: 1 or residues corresponding thereto and is for use with any PPO-inhibiting herbicide disclosed herein, including for example compound D. In one embodiment of such methods, the plant of the invention may comprise a PPO enzyme having a mutation at positions 365, 404 and 479 of SEQ ID NO: 1 or residues corresponding thereto and is for use with any PPO-inhibiting herbicide disclosed herein, including for example compound E.

In one embodiment of such methods, the plant of the invention may comprise a PPO enzyme having a mutation at positions 305, 404 and 479 of SEQ ID NO: 1 or residues corresponding thereto (e.g. SEQ ID NO 137) and is for use with any PPO-inhibiting herbicide disclosed herein, including for example compound A. In one embodiment of such methods, the plant of the invention may comprise a PPO enzyme having a mutation at positions 365, 404 and 479 of SEQ ID NO: 1 or residues corresponding thereto and is for use with any PPO-inhibiting herbicide disclosed herein, including for example compound B. In one embodiment of such methods, the plant of the invention may comprise a PPO enzyme having a mutation at positions 365, 404 and 479 of SEQ ID NO: 1 or residues corresponding thereto and is for use with any PPO-inhibiting herbicide disclosed herein, including for example compound C. In one embodiment of such methods, the plant of the invention may comprise a PPO enzyme having a mutation at positions 365, 404 and 479 of SEQ ID NO: 1 or residues corresponding thereto and is for use with any PPO-inhibiting herbicide disclosed herein, including for example compound D. In one embodiment of such methods, the plant of the invention may comprise a PPO enzyme having a mutation at positions 365, 404 and 479 of SEQ ID NO: 1 or residues corresponding thereto and is for use with any PPO-inhibiting herbicide disclosed herein, including for example compound E.

Undesired vegetation may include, for example, dicotyledonous and monocotyledonous weeds. Dicotyledonous weeds include, but are not limited to, weeds of the genera: Sinapis, Lepidium, Galium, Slellaria, Matricaria, Anthemis, Galinsoga, Chenopodium, Urtica, Senecio, Amaranthus, Portulaca, Xanthium, Convolvulus, Ipomoea, Polygonum, Sesbania, Ambrosia, Cirsium, Carduus, Sonchus, Solanum, Rorippa, Rotala, Lindernia, Lamium, Veronica, Abutilon, Emex, Datura, Viola, Galeopsis, Papaver, Centaurea, Trifolium, Ranunculus, and Taraxacum. Monocotyledonous weeds include, but are not limited to, weeds of the genera: Echinochloa, Setaria, Panicum, Digitaria, Phleum, Poa, Festuca, Eleusine, Brachiaria, Lolium, Bromus, Avena, Cyperus, Sorghum, Agropyron, Cynodon, Monochoria, Fimbristyslis, Sagittaria, Eleocharis, Scirpus, Paspalum, Ischaemum, Sphenoclea, Dactyloctenium, Agrostis, Alopecurus, and Apera.

In addition, undesired vegetation can include, for example, crop plants that are growing in an undesired location. For example, a volunteer maize plant that is in a field that predominantly comprises soybean plants can be considered a weed, if the maize plant is undesired in the field of soybean plants.

An “effective amount” or “effective concentration” refers to an amount and concentration, respectively, of a compound that inhibits PPO enzymatic activity (a PPO-inhibiting herbicide), that is sufficient to kill or inhibit the growth of a wild-type plant and/or undesired plant, plant tissue, plant cell, microspore, or host cell, but that said amount does not kill or inhibit as severely the growth of the at least partially resistant plants, parts thereof, plant tissues, plant cells, and seeds having the modified PPOs described herein. Typically, the effective amount is an amount that is routinely used in agricultural production systems to kill unwanted vegetation of interest. Such an amount is known to those of ordinary skill in the art, or can be easily determined using methods known in the art. Furthermore, it is recognized that the effective amount in an agricultural production system might be substantially different than an effective amount for a plant culture system such as, for example, the microspore culture system. Thus, the amount may be small enough to simply retard or suppress the growth or development of a given weed or undesired vegetation, or the amount may be large enough to irreversibly destroy a given weed or the undesired vegetation. Further, the amount may be any amount there-between.

An effective amount may be at least 1 gram of active compound per hectare (g ai/ha). For example a compound that inhibits PPO enzymatic activity may be applied at a concentration of at least 1, 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500 grams of active compound per hectare.

It should be understood that in the aforementioned methods the compound may be applied to the vicinity of the plant pre-emergence of the crop and/or post-emergence of the crop—a so-called “over-the-top” application.

“Preemergent” refers to a compound which is applied to the vicinity of an at least partially resistant plant of the invention (e.g., a field or area of cultivation) before the plant emerges visibly from the soil and/or before germination of a seed. “Postemergent” refers to an compound which is applied to the vicinity of an at least partially resistant plant of the invention after a plant emerges visibly from the soil. In some instances, the terms “preemergent” and “postemergent” are used with reference to a weed or undesired vegetation in the vicinity of an at least partially resistant plant of the invention, and in some instances these terms are used with reference to a crop plant in the vicinity of an at least partially resistant plant of the invention. When used with reference to a weed or undesired vegetation, these terms may apply to only a particular type of weed or species of weed or undesired vegetation that is present or believed to be present in the area of interest. While any compound which inhibits PPO enzymatic activity may be applied in a preemergent and/or postemergent treatment, some such compounds are known to be more effective in controlling a weed or weeds or undesired plants when applied either preemergence or postemergence. The compound may be applied “preplant incorporation” which involves the incorporation of the compound into the soil prior to planting.

The rates of application of a compound which inhibits PPO enzymatic activity may vary within wide limits and depend on the nature of the soil, the method of application (pre-emergence; post-emergence; application to the seed furrow; no tillage application etc.), the plant, the undesired vegetation to be controlled, the prevailing climatic conditions, and other factors governed by the method of application, the time of application and the target plant. The compound which inhibits PPO enzymatic activity may be applied at a rate of at least 1 L/ha. For example at least 1, 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, 2000 L/ha. In some examples, the compound may be applied at a rate of 200 L/ha.

The application is generally made by spraying the compound, typically by tractor mounted sprayer for large areas, but other methods such as dusting (for powders), drip or drench can also be used.

As discussed elsewhere herein, the plants and plant parts and seeds are provided having stably incorporated into their genome a modified PPO provided herein, as well as, an additional polynucleotide conferring tolerance to at least one additional herbicide, including for example, herbicides comprising glyphosate, glufosinate, HPPD herbicides, and others.

As such, the application of an effective amount of a PPO-inhibiting herbicide can be combined with the application of other herbicides to which the crop is naturally tolerant or to which the crop is resistant via expression of a one or more modified herbicide resistance genes.

The one or more other herbicides that are applied in combination with the PPO inhibiting herbicide can be applied sequentially (e.g., in succession) or simultaneously without affecting the yield or growth of the herbicide resistant plant. This includes applying the one or more other herbicides in accordance with a schedule based on the application of the PPO inhibiting herbicide. When applied simultaneously, an effective amount of each herbicide is applied to the plant at the same time (e.g., at the same stage of plant growth, and/or as constituents of a herbicide composition comprising the one or more of herbicides and the HPPD inhibiting herbicide). When applied sequentially, an effective amount of each herbicide is applied to the plant in succession.

According to the present invention, “enhancing plant growth of a plant” means an improvement in plant vigour, an improvement in plant quality, improved tolerance to stress factors, and/or improved input use efficiency.

The invention described herein also relates to a kit comprising a container and instructions for use, the container comprising a compound which inhibits PPO enzymatic activity, and the instructions comprising a direction to apply the compound to a plant modified to comprise a modified PPO enzyme that provides the plant or part thereof with increased resistance to said compound.

Suitably the compound may be any one of those as defined above. Suitably the direction to apply the compound may comprise direction to apply the compound in an effective amount as defined above. Suitably the direction to apply the compound may comprise direction to apply the compound at a particular rate as described above. Suitably the direction to apply the compound may comprise direction to apply the compound in a particular method such as by spraying as defined above. Suitably the direction to apply the compound may comprise direction to apply the compound at a particular time such as pre-emergence of the crop and/or post-emergence of the crop, or during a particular season or month.

Suitably the instructions may further comprise direction to prepare the compound such that it can be applied in the desired effective amount and by the desired method. Suitably such directions may include direction to dilute the compound, suitably in a solvent such as water, suitably to an effective concentration.

Further provided are compositions comprising a modified PPO enzyme provided herein (such as any of SEQ ID NOs: 1, 2, 4-151, 153-302, or 305-336 or SEQ ID Nos: 37-39, 58, 59, 97-99, 118, 119, 125-137, 139-151, 188-190, 209, 210, 248-250, 269, 270, 277-288 or 291-302 or active variant thereof) and a PPO inhibiting herbicide, including but not limited to the herbicides disclosed herein as herbicide A, B, C, D, or E.

### Methods of Screening

Further provided are various methods of screening for plants having an increased resistance to a compound which inhibits PPO enzymatic activity, for modified PPO enzyme which comprises an increased resistance to a compound which inhibits PPO enzymatic activity, and also for compounds which inhibits PPO enzymatic activity.

In one aspect of the invention there is provided a method of selecting a plant or organism (including, for example, a prokaryotic organism such as a bacteria) having an increased resistance to a compound which inhibits PPO enzymatic activity relative to an unmodified plant or organism, comprising: (a) providing a plant or organism; (b) optionally mutagenizing the plant or organism; (c) exposing the plant or organism to an effective amount of a compound which inhibits PPO enzymatic activity; and (d) selecting the plant or organism if the plant or organism displays resistance to the compound.

In one embodiment, the plant may be a plant according to the first aspect of the invention, and may comprise any of the features defined in relation to such a plant recited herein.

Suitably step (b) may comprise mutagenizing the plant or organism by any known means such as EMS or other chemical treatment, or by X-ray or another radiation treatment.

Suitably to induce modifications, suitably one or more mutations, in the PPO gene.

Suitably after step (b), the plant or organism comprises one or more modifications to the endogenous or a heterologous PPO gene, suitably one or more mutations to the endogenous or the heterologous PPO gene. Suitably the modified PPO gene may encode a modified PPO enzyme, suitably as described herein. Suitably therefore, after step (b) the plant or organism may comprise a modified PPO enzyme as described herein, suitably having one or more mutations as described herein.

Suitably step (c) comprises exposing the plant or organism to an effective amount of a compound as described herein above. Suitably to determine if the one or more modifications to the endogenous or heterologous PPO gene, and the resulting modified PPO enzyme, created in step (b) confer resistance to the compound.

Suitably selecting the plant or organism if the plant or organism displays resistance to the compound may comprise, for example, for plants selecting those plants that do not display any signs of PPO deficiency. Suitably such signs may include stunted growth, wilting, necrosis, discolouration, and the like. For organisms, such as bacteria, resistance to the compound may comprise the ability to grow in the presence of the herbicide.

Further provided is a method of identifying a modified PPO enzyme which comprises an increased resistance to a compound which inhibits PPO enzymatic activity, comprising: (a) generating a library of modified PPO encoding polynucleotides; (b) screening a population of the resulting modified PPO encoding polynucleotides by expressing each of said polynucleotides in a bacteria, a plant or a plant part and exposing the bacteria, plant or part thereof to an effective amount of a compound which inhibits PPO enzymatic activity; (c) selecting the modified PPO encoding polynucleotides which provide the bacteria, plant or plant part thereof with increased resistance to said compound compared to a reference bacteria, plant or plant part thereof containing an unmodified or control PPO encoding polynucleotide.

Suitably generating a library of modified PPO encoding polynucleotides may be carried out by any known technique to generate a library of genes comprising different modifications, suitably mutations throughout the gene sequence. Suitably these methods may be random or directed. Such methods may include a step of exposing the PPO encoding polynucleotides to a mutagen, such as a chemical or radiation, or carrying out error prone PCR on the PPO encoding polynucleotides for example. Other molecular techniques may include performing DNA shuffling or staggered extension processes on PPO encoding polynucleotides.

Suitably expressing the modified polynucleotides in a bacteria, a plant or a plant part, such that the modified PPO enzymes are expressed, comprises transforming the bacteria, a plant or a plant part with each of the modified polynucleotides. Suitable means of transformation are described elsewhere herein, as are suitable constructs for expression of a PPO encoding polynucleotide.

Suitably exposing the bacteria, plant or part thereof to an effective amount of a compound which inhibits PPO enzymatic activity comprises using an effective amount as described hereinabove, of a suitable compound as described hereinabove.

Suitably selecting the modified PPO encoding polynucleotides which provide the bacteria, plant or plant part thereof with increased resistance to said compound, comprises selecting those host bacteria, plants or plant parts thereof that display resistance to the compound, suitably selecting those that do not display any signs of PPO deficiency. Suitably such signs in plants may include stunted growth, wilting, necrosis, discolouration, and the like. Suitably such signs in bacteria may include stunted growth or death. In one embodiment, the sign is reduced growth compared to an unmodified or control bacteria or unmodified or control plant.

Also provided is a method of identifying a compound which inhibits PPO enzymatic activity comprising: (a) generating a modified plant or part as described herein, (b) applying a test compound to the plant or part thereof of step (a) and to an unmodified reference plant; (c) selecting the test compounds which confer reduced growth to the unmodified reference plant as compared to the growth of the modified plant or part thereof.

In one embodiment, the modified plant or plant part is a plant having increased resistance to compounds which inhibit PPO enzymatic activity, as defined according to the first aspect of the invention, and may comprise any of the features defined in relation to such a plant recited herein.

Suitably the test compound may be any compound which may have, or which is expected to have an inhibitory effect on a PPO enzyme of the plant. Suitably the test compound may be related to, derived from, or synthesised from a compound as defined herein, which may be a known compound which inhibits PPO enzymatic activity, such as a known herbicidal compound.

Suitably the test compound may be applied in a test amount, suitably the test amount may be the same as any known effective amount for such herbicidal compounds, suitable amounts are defined hereinabove.

In another aspect of the invention, there are provided one or more compounds identified by such a method, optionally for use as herbicides.

### FIGURES

The invention will now be described with reference to the follow figures in which:

FIG. 1 shows a plasmid map of pBin TMV AtPPO1 transformation vector.

### EXAMPLES

### Example 1—Expression and Assay of an Arabidopsis thaliana PPO1 Site Saturation Variant Library in E. coli

A pET24a plasmid containing a DNA sequence encoding a variant of PPO1 derived from Arabidopsis thaliana (AtPPO1), codon-optimised for expression in E. coli and under control of a T7 promoter, was synthesised by Twist Bioscience (SEQ ID NO:3). This plasmid was used as the basis of a Site Saturation Variant Library (SSVL) that substituted the codons at 352 amino acid positions for codons that encoded one of the other 19 amino acids, or removed the codon altogether, resulting in a translation product with a deletion at that position. The 352 positions that were mutated are listed in table 1.

TABLE 1
AtPPO1 amino acid positions mutated with reference to SEQ ID NO 1

| | | | | | | | |
|---|---|---|---|---|---|---|---|
| I61 | L131 | R200 | P271 | L357 | Y407 | P463 | E526 |
| V62 | K132 | G203 | Q272 | S358 | S408 | L464 | V527 |
| G63 | D133 | D204 | G273 | K359 | S409 | K465 | |
| G64 | D134 | E205 | Q274 | L360 | S410 | L466 | |
| G65 | L135 | V206 | T275 | Y361 | L411 | G467 | |
| I66 | V136 | F207 | V276 | Y362 | F412 | V468 | |
| S67 | L137 | E208 | G277 | P363 | P413 | R469 | |
| G68 | G138 | R209 | S278 | P364 | N414 | V470 | |
| L69 | D139 | L210 | F279 | V365 | R415 | W471 | |
| C70 | P140 | I211 | R280 | A366 | A416 | P472 | |
| I71 | T141 | E212 | K281 | A367 | P417 | Q473 | |
| A72 | A 142 | P213 | G282 | V368 | P418 | A474 | |
| Q73 | P143 | F214 | L283 | S369 | G419 | I475 | |
| A74 | R144 | C215 | R284 | I370 | R420 | P476 | |
| V88 | F145 | S216 | M285 | S371 | I421 | Q477 | |
| T89 | V146 | G217 | L286 | Y372 | L422 | F478 | |
| E90 | L147 | V218 | P287 | P373 | L423 | L479 | |
| A91 | W148 | Y219 | E288 | K374 | L424 | V480 | |
| K92 | N149 | A220 | A289 | E375 | N425 | G481 | |
| D93 | G150 | G221 | I290 | A376 | Y426 | H482 | |
| R94 | K151 | D222 | S291 | I377 | I427 | F483 | |
| V95 | L152 | P223 | A292 | R378 | G428 | D484 | |
| G96 | R153 | S224 | R293 | T379 | G429 | I485 | |
| G97 | P154 | K225 | L294 | E380 | S430 | L486 | |
| N98 | V155 | L226 | V298 | C381 | T431 | D487 | |
| I99 | P156 | S227 | K299 | L382 | N432 | T488 | |
| I100 | S157 | M228 | L300 | I383 | T433 | A489 | |
| T101 | K158 | K229 | S301 | D384 | G434 | L503 | |
| R102 | L159 | A230 | W302 | G385 | I435 | G504 | |
| E103 | T160 | A231 | K303 | E386 | L436 | G505 | |
| F107 | D161 | F232 | L304 | L387 | S437 | N506 | |
| L108 | L162 | G233 | S305 | K388 | E442 | Y507 | |
| W109 | P163 | K234 | E320 | G389 | L443 | V508 | |
| E110 | F164 | V235 | T321 | F390 | A446 | A509 | |
| E111 | F165 | W236 | V333 | G391 | V447 | G510 | |
| G112 | D166 | L238 | V334 | Q392 | D448 | V511 | |
| P113 | L167 | I246 | M335 | L393 | R449 | A512 | |
| N114 | M168 | G248 | T336 | H394 | D450 | L513 | |
| S115 | S169 | T249 | V337 | P395 | L451 | G514 | |
| F116 | G172 | F250 | P338 | R396 | R452 | R515 | |
| Q117 | K173 | K251 | S339 | T397 | K453 | C516 | |
| P118 | A176 | A252 | H340 | Q398 | M454 | V517 | |
| S119 | G179 | I253 | V341 | G399 | L455 | E518 | |
| D120 | S193 | Q254 | A342 | V400 | I456 | G519 | |
| P121 | V194 | D265 | S343 | E401 | K457 | A520 | |
| M122 | E195 | P266 | G344 | T402 | P458 | Y521 | |
| L123 | E196 | R267 | A353 | L403 | N459 | E522 | |
| T124 | F197 | L268 | A354 | G404 | S460 | T523 | |
| V126 | V198 | P269 | N355 | T405 | T461 | A524 | |
| V127 | R199 | K270 | A356 | I406 | D462 | I525 | |

The SSVL was batch-transformed by electroporation into an E. coli BL21(DE3) hemG strain with the hemG gene replaced by CmR conferring resistance to chloramphenicol and spread onto LA supplemented with 50 μg/mL kanamycin, 25 μg/mL chloramphenicol and 20 μg/mL hematin. Each batch consisted of all the plasmids that contained variants at two sites.

Colonies from batch transformations were inoculated into 500 μL of Terrific broth supplemented with 50 μg/mL kanamycin, 25 μg/mL chloramphenicol and 20 μg/mL hematin in 96-deepwell blocks. Cultures were grown for 24 hours at 37° C. with shaking at 250 rpm.

Overnight cultures were centrifuged and the cell pellets resuspended with 500 μL of Terrific broth. The resuspended cultures were used at a ratio of 1:100 to inoculate a fresh 96-deepwell block containing 500 μL of Terrific broth supplemented with 0.1 mM IPTG, 50 μg/mL kanamycin, 25 μg/mL chloramphenicol and 6.25 ppm of herbicide 0 or E or 0.78 ppm herbicide B or DMSO. Assay cultures were grown for 24 hours at 37° C. with shaking at 250 rpm with the OD600 being measured at 24 hours post inoculation. Strains that grew in both DMSO and herbicide were determined to be herbicide tolerant with the percentage of growth inhibition being calculated as below:

$$OD 600 DMSO - OD 600 herbicide / OD 600 DMSO \times 100$$

Plasmids were isolated from herbicide-tolerant strains and sequenced to identify the causal mutation in the AtPPO1 gene carried by pET24a in each strain. All herbicide-tolerant variants identified are in Table 2.

TABLE 2
AtPPO1 variants that were identified as being resistant to one or more of
herbicides B, D or E. The table shows the percentage growth inhibition
in the presence of herbicides B, D or E compared to growth in DMSO.

| | AtPPO1 | Herbicide B | Herbicide D | Herbicide E |
|---|---|---|---|---|
| SEQ ID | variant | (0.78 ppm) | (6.25 ppm) | (6.25 ppm) |
| SEQ ID | WT | 61 | 58 | 64 |
| NO: 1 | | | | |
| SEQ ID | G64A | 36 | 38 | 34 |
| NO: 4 | | | | |
| SEQ ID | V88I | 32 | 43 | 66 |
| NO: 5 | | | | |
| SEQ ID | E111D | 23 | 30 | 69 |
| NO: 6 | | | | |
| SEQ ID | E111F | 27 | 29 | 66 |
| NO: 7 | | | | |
| SEQ ID | V127I | 28 | 23 | 69 |
| NO: 8 | | | | |
| SEQ ID | V127L | 31 | 29 | 65 |
| NO: 9 | | | | |
| SEQ ID | P154Y | 34 | 38 | 68 |
| NO: 10 | | | | |
| SEQ ID | K173H | 29 | 34 | 55 |
| NO: 11 | | | | |
| SEQ ID | A176L | 35 | 42 | 61 |
| NO: 12 | | | | |
| SEQ ID | V198W | 30 | 47 | 51 |
| NO: 13 | | | | |
| SEQ ID | V206G | 32 | 19 | 58 |
| NO: 14 | | | | |
| SEQ ID | F207Y | 29 | 19 | 66 |
| NO: 15 | | | | |
| SEQ ID | E212Y | 25 | 27 | 65 |
| NO: 16 | | | | |
| SEQ ID | F214Y | 26 | 34 | 63 |
| NO: 17 | | | | |
| SEQ ID | V218K | 38 | 31 | 50 |
| NO: 18 | | | | |
| SEQ ID | Y219S | 28 | 33 | 49 |
| NO: 19 | | | | |
| SEQ ID | A220C | 15 | 33 | 66 |
| NO: 20 | | | | |
| SEQ ID | P271C | 24 | 24 | 66 |
| NO: 21 | | | | |
| SEQ ID | Q272H | 27 | 28 | 64 |
| NO: 22 | | | | |
| SEQ ID | Q272I | 24 | 29 | 63 |
| NO: 23 | | | | |
| SEQ ID | Q272T | 28 | 23 | 64 |
| NO: 24 | | | | |
| SEQ ID | K281C | 35 | 33 | 54 |
| NO: 25 | | | | |
| SEQ ID | K281E | 37 | 33 | 61 |
| NO: 26 | | | | |
| SEQ ID | K281N | 39 | 37 | 56 |
| NO: 27 | | | | |
| SEQ ID | R284G | 35 | 37 | 60 |
| NO: 28 | | | | |
| SEQ ID | S305L | 26 | 14 | 39 |
| NO: 29 | | | | |
| SEQ ID | L360C | 25 | 21 | 33 |
| NO: 30 | | | | |
| SEQ ID | Y361D | 20 | 33 | 14 |
| NO: 31 | | | | |
| SEQ ID | Y361F | 31 | 37 | 50 |
| NO: 32 | | | | |
| SEQ ID | Y361P | 28 | 36 | 38 |
| NO: 33 | | | | |
| SEQ ID | Y361Q | 19 | 32 | 0 |
| NO: 34 | | | | |
| SEQ ID | Y361R | 36 | 43 | 56 |
| NO: 35 | | | | |
| SEQ ID | Y361T | 37 | 44 | 37 |
| NO: 36 | | | | |
| SEQ ID | Y362F | 34 | 13 | 43 |
| NO: 37 | | | | |
| SEQ ID | V365L | 11 | 55 | 27 |
| NO: 38 | | | | |
| SEQ ID | V365M | 3 | 42 | 22 |
| NO: 39 | | | | |
| SEQ ID | G404A | 12 | 4 | 2 |
| NO: 40 | | | | |
| SEQ ID | P413G | 16 | 32 | 59 |
| NO: 41 | | | | |
| SEQ ID | P418M | 12 | 25 | 64 |
| NO: 42 | | | | |
| SEQ ID | L424V | 19 | 33 | 66 |
| NO: 43 | | | | |
| SEQ ID | Y426C | 2 | 34 | 4 |
| NO: 44 | | | | |
| SEQ ID | Y426L | 15 | 25 | 12 |
| NO: 45 | | | | |
| SEQ ID | Y426M | 7 | 25 | 17 |
| NO: 46 | | | | |
| SEQ ID | Y426T | 8 | 12 | 11 |
| NO: 47 | | | | |
| SEQ ID | Y426V | 5 | 16 | 7 |
| NO: 48 | | | | |
| SEQ ID | T431R | 25 | 28 | 56 |
| NO: 49 | | | | |
| SEQ ID | V447C | 34 | 32 | 53 |
| NO: 50 | | | | |
| SEQ ID | V447I | 35 | 36 | 61 |
| NO: 51 | | | | |
| SEQ ID | D448H | 31 | 26 | 57 |
| NO: 52 | | | | |
| SEQ ID | D448L | 38 | 38 | 54 |
| NO: 53 | | | | |
| SEQ ID | D448N | 40 | 38 | 63 |
| NO: 54 | | | | |
| SEQ ID | R449K | 35 | 37 | 68 |
| NO: 55 | | | | |
| SEQ ID | T461Q | 31 | 22 | 35 |
| NO: 56 | | | | |
| SEQ ID | F478R | 23 | 30 | 61 |
| NO: 57 | | | | |
| SEQ ID | L479M | 29 | 4 | 28 |
| NO: 58 | | | | |
| SEQ ID | L479N | 23 | 3 | 31 |
| NO: 59 | | | | |
| SEQ ID | G510N | 24 | 31 | 70 |
| NO: 60 | | | | |
| SEQ ID | V511L | 18 | 18 | 46 |
| NO: 61 | | | | |
| SEQ ID | V511N | 20 | 22 | 48 |
| NO: 62 | | | | |
| SEQ ID | C516Q | 18 | 28 | 51 |
| NO: 63 | | | | |
| SEQ ID | K270M | 28 | 23 | 29 |
| NO: 305 | | | | |
| SEQ ID | Y361C | 36 | 15 | 35 |
| NO: 306 | | | | |
| SEQ ID | Y362C | 37 | 43 | 58 |
| NO: 307 | | | | |
| SEQ ID | T431F | 41 | 0 | 37 |
| NO: 308 | | | | |
| SEQ ID | L464Y | 0 | 12 | 5 |
| NO: 309 | | | | |
| SEQ ID | G481D | 40 | 20 | 16 |
| NO: 310 | | | | |
| SEQ ID | G481K | 10 | 12 | 0 |
| NO: 311 | | | | |
| SEQ ID | G481N | 44 | 14 | 4 |
| NO: 312 | | | | |

Note
throughout the examples Herbicides A to E are as described above.

### Example 2—AtPPO1 Combinatorial Variant Library Screening

A subset of identified herbicide tolerance mutations from Example 1 were selected to be incorporated into a combinatorial variant library (produced by Twist Bioscience). The selected mutations and designed frequency within the combinatorial library are shown in Table 3.

TABLE 3
AtPPO1 mutations selected for inclusion in a combinatorial library. A
percentage frequency of each mutation within the library is shown.

| Amino acid | | | | | |
|---|---|---|---|---|---|
| position in | | | | | |
| AtPPO1 | | | | | |
| (SEQ ID | | VARIANT | VARIANT | VARIANT | VARIANT |
| NO: 1) | WT | 1 | 2 | 3 | 4 |
| 270 | K (95%) | M (5%) | | | |
| 305 | S (75%) | L (25%) | | | |
| 360 | L (95%) | C (5%) | | | |
| 361 | Y (75%) | C (6.25%) | D (6.25%) | Q (6.25%) | T (6.25%) |
| 362 | Y (95%) | F (5%) | | | |
| 365 | V (75%) | L (12.5%) | M (12.5%) | | |
| 404 | G (75%) | A (25%) | | | |
| 426 | Y (75%) | C (6.25%) | M (6.25%) | T (6.25%) | V (6.25%) |
| 431 | T (95%) | F (2.5%) | R (2.5%) | | |
| 461 | T (95%) | Q (5%) | | | |
| 464 | L (95%) | Y (5%) | | | |
| 479 | L (95%) | M (2.5%) | N (2.5%) | | |
| 481 | G (75%) | D (8.33%) | K (8.33%) | N (8.33%) | |

The combinatorial library was transformed into E. coli BL21(DE3) hemG and selected for increased tolerance to herbicides as described in Example 1 except for the herbicide concentrations being increased to 25 ppm (herbicides B and D) and 50 ppm (herbicide E). Plasmids were isolated from herbicide-tolerant strains and sequenced to identify the causal mutation in the AtPPO1 gene carried by the pET24 plasmid in each strain. All herbicide-tolerant variants identified are in Table 4.

TABLE 4
AtPPO1 variants with multiple mutations selected on the basis
of strong growth in the presence of herbicide.

| | | |
|---|---|---|
| SEQ ID NO: 124 | S305L Y426M (comparator) | |
| SEQ ID NO: 125 | S305L Y426V | AtPPO1 V030 |
| SEQ ID NO: 126 | V365L Y426V L479N | AtPPO1 V004 |
| SEQ ID NO: 127 | S305L G404A Y426V T431R | AtPPO1 V036 |
| | L479N | |
| SEQ ID NO: 128 | S305L V365M | AtPPO1 V055 |
| SEQ ID NO: 129 | S305L Y362F G404A | AtPPO1 V018 |
| SEQ ID NO: 130 | S305L Y361T V365L T431R | AtPPO1 V020 |
| | L479M | |
| SEQ ID NO: 131 | S305L Y426V L479M | AtPPO1 V002 |
| SEQ ID NO: 132 | Y426M T461Q L479M | AtPPO1 V025 |
| SEQ ID NO: 133 | S305L Y361D V365M | AtPPO1 V052 |
| SEQ ID NO: 134 | S305L Y361C Y362F Y426M | AtPPO1 V031 |
| | L479M | |
| SEQ ID NO: 135 | Y361D V365L | AtPPO1 V035 |
| SEQ ID NO: 136 | V365L G404A L479N | AtPPO1 V022 |
| SEQ ID NO: 137 | S305L G404A L479N | AtPPO1 V019 |

### Example 3—Expression and Assay of Selected Arabidopsis thaliana PPO1 Variants and Percent-Inhibition Values Versus Herbicides A to E

A pET24 plasmid containing a DNA sequence encoding each identified variant mutant of AtPPO1 derived from Arabidopsis thaliana, codon-optimised for expression in E. coli, and under control of a T7 promoter, was synthesised by Twist Bioscience. This plasmid was expressed in E. coli BL21 (DE3) hemG with 50 μg/mL kanamycin and 25 μg/mL chloramphenicol selection. Overnight cultures grown at 37° C. were used to inoculate 150 ml AIM (AIM-Terrific Broth Base including Trace elements, Formedium) in 500 ml shake flasks at a ratio of 1:100. Cultures were grown at 37° C., 200 rpm for 3 hours and then overnight at 26° C. Cells were harvested by 4° C. centrifugation for 20 minutes at 600 rpm. Cells were stored at −80° C. until extraction. 0.5-1 g of cell pellet from was thawed and resuspended in ratio of 1:1 in lysis buffer (20 mM Tris pH8, 500 mM sodium chloride, 10% glycerol, 1× complete Protease EDTA-free Inhibitor Tablet (Roche)). The resuspension was sonicated at 50% using 10-20 cycles of 10 seconds, 20 seconds off. After sonication Triton X-100 was added to the lysates at a final concentration of 0.5%. The lysates were centrifuged at 13.00 rpm for 10 min at 4° C. The crude extracts (i.e, supernatants) were stored at −80° C. Protein concentration was measured and calculated using the absorbance at 280 nm. Extracts were diluted 1 in 4 and 10 uL was analysed by SDS PAGE.

Variant crude extracts were assayed in a fluorescent Protoporphyrinogen Oxidase assay in black 384 well plates with a clear bottom. The total volume of the reaction mixture was 100 ul and contained 100 mM K2HPO4 pH7.2, 1 mM Na2EDTA, 5 mM DTT, 20% glycerol (v/v), 0.025% Tween 20 (v/v). Reactions were started with addition of 1 uM protoporphyrinogen IX final assay concentration. Fluorescence was monitored over 30 min using a Tecan plate reader with an excitation wavelength of 405 nm, emission wavelength of 635 nm, gain of 90 and 15 flashes. 10 ul of 3-fold serial dilution of crude extract in 100 mM K2HPO4 pH7.2, 1 mM Na2EDTA, 5 mM DTT, 20% glycerol (v/v), 0.05% Tween 20 (v/v) was tested in the assay against 1% DMSO and 100 ppm herbicide B to determine the correct amount of crude extract to add to the assay to observe activity. 10 ul of the appropriate crude extract dilution was then tested in the fluorescent Protoporphyrinogen Oxidase assay against DMSO and 10-fold dose response of each compound (herbicides A to E) with a top rate of 100 ppm.

The percentage inhibition was calculated as the activity in the presence of one of four concentrations of each herbicide compound A to E (note that the compounds labelled as A to E are identified hereinabove in the relevant section of the description) as a percentage of the enzyme activity in DMSO (Table 5).

TABLE 5

| | DMSO | | | | Herbicide A | | | | Herbicide B | | | | Herbicide C | | | | Herbicide D | | | | Herbicide E | | | |
|---|---|---|---|---|---|---|---|---|---|---|---|---|---|---|---|---|---|---|---|---|---|---|---|---|
| | 0.1 | 1 | 10 | 100 | 0.1 | 1 | 10 | 100 | 0.1 | 1 | 10 | 100 | 0.1 | 1 | 10 | 100 | 0.1 | 1 | 10 | 100 | 0.1 | 1 | 10 | 100 |
| WT (SEQ ID NO.1) | 0 | 0 | 0 | 1 | 94 | 100 | 97 | 100 | 97 | 97 | 96 | 100 | 91 | 91 | 98 | 93 | 91 | 90 | 96 | 100 | 91 | 97 | 100 | 100 |
| S305L Y426M (SEQ ID NO.124) | 0 | 0 | 1 | 4 | 34 | 82 | 100 | 100 | 100 | 100 | 100 | 100 | 77 | 88 | 100 | 100 | 53 | 85 | 100 | 100 | 38 | 78 | 100 | 96 |
| S305L Y426V (SEQ ID NO.125) | 0 | 0 | 0 | 5 | 0 | 11 | 63 | 87 | 87 | 99 | 100 | 100 | 0 | 31 | 81 | 97 | 0 | 10 | 53 | 83 | 0 | 2 | 50 | 84 |
| V365L Y426V L479N | 1 | 0 | 3 | 0 | 0 | 36 | 73 | 90 | 98 | 100 | 100 | 100 | 4 | 44 | 82 | 93 | 12 | 33 | 89 | 98 | 0 | 22 | 77 | 94 |
| (SEQ ID NO.126) | | | | | | | | | | | | | | | | | | | | | | | | |
| S305L G404A Y426V T431R | 0 | 0 | 8 | 6 | 9 | 45 | 69 | 88 | 91 | 100 | 100 | 100 | 47 | 57 | 83 | 96 | 13 | 40 | 75 | 92 | 11 | 48 | 75 | 92 |
| L479N (SEQ ID NO.127) | | | | | | | | | | | | | | | | | | | | | | | | |
| S305L V365M (SEQ ID NO.128) | 12 | 2 | 1 | 0 | 41 | 64 | 92 | 91 | 100 | 97 | 95 | 100 | 45 | 76 | 95 | 100 | 51 | 84 | 96 | 100 | 0 | 71 | 91 | 100 |
| S305L Y362F G404A | 0 | 3 | 0 | 4 | 25 | 78 | 92 | 100 | 70 | 93 | 97 | 100 | 24 | 76 | 93 | 100 | 9 | 45 | 83 | 96 | 15 | 53 | 84 | 100 |
| (SEQ ID NO.129) | | | | | | | | | | | | | | | | | | | | | | | | |
| S305L Y361T V365L T431R | 7 | 7 | 0 | 0 | 15 | 69 | 99 | 94 | 41 | 100 | 97 | 98 | n.t. | 78 | 99 | 97 | 12 | 42 | 89 | 92 | 9 | 45 | 82 | 91 |
| L479M (SEQ ID NO.130) | | | | | | | | | | | | | | | | | | | | | | | | |
| S305L Y426V L479M | 0 | 0 | 5 | 2 | 0 | 52 | 87 | 100 | 99 | 96 | 100 | 100 | 28 | 59 | 90 | 100 | 4 | 46 | 76 | 97 | 0 | 28 | 70 | 87 |
| (SEQ ID NO.131) | | | | | | | | | | | | | | | | | | | | | | | | |
| Y426M T461Q L479M | 7 | 7 | 0 | 0 | 10 | 51 | 77 | 100 | 100 | 100 | 100 | 100 | 26 | 54 | 75 | 100 | 58 | 79 | 95 | 100 | 0 | 26 | 70 | 100 |
| (SEQ ID NO.132) | | | | | | | | | | | | | | | | | | | | | | | | |
| S305L L361D V365M | 1 | 0 | 5 | 1 | 9 | 53 | 84 | 100 | 96 | 100 | 100 | 100 | 30 | 56 | 92 | 100 | 37 | 87 | 99 | 100 | 0 | 10 | 76 | 88 |
| (SEQ ID NO.133) | | | | | | | | | | | | | | | | | | | | | | | | |
| S305L Y361C L362F Y426M | 0 | 9 | 4 | 0 | 0 | 40 | 83 | 92 | 100 | 100 | 100 | 100 | 19 | 65 | 99 | 100 | 0 | 56 | 85 | 100 | 0 | 39 | 80 | 99 |
| L479M (SEQ ID NO.134) | | | | | | | | | | | | | | | | | | | | | | | | |
| Y361D V365L (SEQ ID NO.135) | 0 | 0 | 0 | 11 | 56 | 89 | 99 | 96 | 82 | 100 | 100 | 100 | 30 | 84 | 94 | 100 | 2 | 63 | 93 | 100 | 0 | 31 | 83 | 100 |
| V365L G404A L479N | 5 | 0 | 0 | 15 | 58 | 95 | 100 | 100 | 79 | 100 | 100 | 100 | 34 | 90 | 99 | 100 | 4 | 75 | 99 | 100 | 0 | 39 | 86 | 100 |
| (SEQ ID NO.136) | | | | | | | | | | | | | | | | | | | | | | | | |
| S305L G404A L479N | 7 | 0 | 2 | 0 | 24 | 93 | 100 | 100 | 77 | 98 | 100 | 100 | 26 | 90 | 100 | 100 | 12 | 58 | 98 | 100 | 22 | 66 | 100 | 96 |
| (SEQ ID NO.137) | | | | | | | | | | | | | | | | | | | | | | | | |

The results show that the selected variants showed improved tolerance to at least one of the PPO inhibiting herbicides A to E compared to the parental gene AtPPO1 (SEQ ID NO:1) or previously known PPO1 variant (SEQ ID NO:2). In some cases, such as for AtPPO1 S305L Y426V, (SEQ ID NO:125) the improvement in herbicide tolerance was seen across all herbicides tested. In other examples differential tolerances are seen to the tested herbicides, such as with AtPPO1 Y361D V365L (SEQ ID NO:135) where the improvement over SEQ ID NO:124 was seen for some of the herbicides (i.e. B, C, D and E) but not for others (i.e. herbicide A).

A pET24 plasmid containing a DNA sequence encoding variants of AtPPO1 derived from Arabidopsis thaliana with cTPs removed and both a 6×His tag and TEV protease cleavage tag added at the N-terminus, codon-optimised for expression in E. coli, and under control of a T7 promoter, was synthesised by Twist Bioscience. The following variants were synthesised: WT AtPPO1 (SEQ ID NO: 370), AtPPO1 L479M (SEQ ID NO: 371), AtPPO1 L479N (SEQ ID NO: 372), AtPPO1 S305L Y426V (SEQ ID NO: 374) and AtPPO1 S305L Y426V L479M (SEQ ID NO: 373). This plasmid was expressed in E. coli BL21 (DE3) with 50 μg/mL kanamycin selection. 10 mL of overnight cultures grown at 37° C. were used to inoculate 1 L AIM (AIM-Terrific Broth Base including Trace elements, Formedium) in 2 L shake flasks. Cultures were grown at 37° C., 200 rpm for 3 hours and then overnight at 26° C. Cells were harvested centrifugation. 5-15 g of cell pellet was lysed in and resuspended in ratio of 1:1.5 in lysis buffer (20 mM Tris pH8, 500 mM sodium chloride, 10% glycerol, 1× complete Protease EDTA-free Inhibitor Tablet (Roche), 10 mM imidazole). The resuspension was sonicated at 75% using 10 cycles of 30 seconds, 40 seconds off. After sonication Triton X-100 was added to the lysates at a final concentration of 0.5%. The lysates were centrifuged at 20,000 rpm for 25 min at 4° C. Cleared lysates were loaded on His GraviTrap (Cytiva) columns, washed with 20 mL of lysis buffer and eluted in elution buffer (20 mM Tris pH8, 500 mM sodium chloride, 10% glycerol, 200 mM imidazole, 0.03% (v/v) Triton X-100). The eluate was desalted on a PD10 column and 2.5-3.2 mL was collected. Protein concentration was measured and calculated using the absorbance at 280 nm. Extracts were diluted 1 in 4 and 10 uL was analysed by SDS PAGE.

Partially purified Protoporphyrinogen Oxidase enzymes were assayed for activity using fluorescence adapted from the method of Shepherd & Dailey (Anal Biochem. 2005 344(1) 115-121). The assay was performed in black 384 well plates with a clear bottom and the total volume of the reaction mixture was 91 ul. The final assay mixture contained 100 mM K2HPO4 pH7.2, 1 mM Na2EDTA, 5 mM DTT, 20% glycerol (v/v), 0.05% Tween 20 (v/v), 1.1% DMSO (v/v), 1 uM protoporphyrinogen IX and 1.5 nM enzyme. Compounds were dissolved in DMSO at 1 mM and a 4-fold serial dilution carried out to give 8 rates. 1 ul was added to the assay mixture. This gave final compound concentrations of 11, 2.7, 0.69, 0.17, 0.04, 0.01, 0.002, 0.0007 uM. Enzyme and inhibitor were preincubated for 5 minutes prior to starting the reaction with the addition of the substrate. Fluorescence was monitored over 30 min at room temperature using a Tecan Infinite M200PRO plate reader with an excitation wavelength of 405 nm, emission wavelength of 635 nm, gain of 100 and 15 flashes. % inhibition relative to uninhibited control was calculated for each compound at each concentration during the linear phase of the reaction. IC50s were calculated using GraphPad Prism 7.05 nonlinear regression (Table 6). Each compound was tested in duplicate.

The IC50s of AtPPO1 V002 (SEQ ID NO: 373) was higher than AtPPO1 V030 (SEQ ID NO: 374) for all herbicides tested other than fomesafen. These variants share two mutations, but AtPPO1 V002 (SEQ ID NO: 373) has an additional L479M substitution that AtPPO V030 (SEQ ID NO: 374) lacks. One mechanism by which substitutions at L479 may enhance tolerance is by increasing the PPO1 enzyme rate of activity (Table 7).

TABLE 6
IC50 (nM) of Protoporphyrinogen Oxidase variants treated
with different PPO inhibitors.

| | His- | His- | His- | His- | His- |
|---|---|---|---|---|---|
| | TEV- | TEV- | TEV- | TEV- | TEV- |
| | AtPP01 | AtPP01 | AtPP01 | AtPPO1 | AtPP01 |
| | ΔcTP | ΔcTP | ΔcTP | ΔcTP | ΔcTP |
| | (SEQ | L479N | L479M | V002 | V030 |
| | ID | (SEQ | (SEQ | (SEQ | (SEQ |
| | NO: | ID NO: | ID NO: | ID NO: | ID NO: |
| | 370) | 372) | 371) | 373) | 374) |
| Herbicide A | 0.7 | 0.9 | 0.8 | 446.7 | 323.2 |
| Herbicide B | 0.7 | 1.0 | 0.8 | 2.1 | 1.3 |
| Herbicide C | 0.7 | 0.9 | 0.8 | 121.0 | 92.0 |
| Herbicide D | 0.5 | 0.8 | 0.6 | 335.1 | 268.9 |
| Herbicide E | 2.1 | 2.6 | 2.4 | 997.6 | 546.9 |
| Azafenadin | 0.8 | 0.8 | 0.7 | 148.7 | 117.0 |
| Butafenacil | 0.6 | 0.9 | 0.7 | 12.5 | 11.0 |
| Flufenoximacil | 0.6 | 0.8 | 0.7 | 863.1 | 717.5 |
| Flumioxazin | 0.8 | 1.0 | 1.0 | 187.4 | 47.2 |
| Fomesafen | 1.8 | 1.4 | 1.2 | 0.5 | 0.7 |
| Oxadiazaon | 19.0 | 6.1 | 11.2 | 5620.0 | 4604.0 |
| Pyraclonil | 2.5 | 1.5 | 2.3 | 5558.0 | 4351.0 |
| Pyraflufen-ethyl | 0.6 | 0.8 | 0.7 | 24.2 | 18.6 |

<1.0 is below detection limit of assay.

TABLE 7
Activity rates of Protoporphyrinogen Oxidase variants in in vitro assay.

| | His- | His-TEV- | His-TEV- | His-TEV- | His-TEV- |
|---|---|---|---|---|---|
| | TEV- | AtPP01 | AtPP01 | AtPPO1 | AtPP01 |
| | AtPP01 | ΔcTP | ΔcTP | ΔcTP | ΔcTP |
| | ΔcTP | L479N | L479M | V002 | V030 |
| | (SEQ ID | (SEQ ID | (SEQ ID | (SEQ ID | (SEQ ID |
| | NO: 370) | NO: 372) | NO: 371) | NO: 373) | NO: 374) |
| Rate (rfu | 5.1 | 13.2 | 9.9 | 7.6 | 5.3 |
| second⁻¹ | | | | | |
| pmole⁻¹) | | | | | |

*rfu = relative fluorescence units

### Example 4—PPO1 Sequences and Expression in Tobacco Plants

Arabidopsis thaliana PPO1 or orthologues of this, for example SEQ ID NOs: 1 and 124 to 137 or Setaria italica PPO1 or orthologues of this, for example SEQ ID NOs: 2 and 138-151 are expressed in transgenic tobacco. DNA sequences that encode these polypeptides (optimized for tobacco or, optionally, codon optimized according to a target crop such as soybean or corn) are prepared synthetically. Each sequence is designed to include a 5′ fusion with TMV omega 5′ leader sequence and such that they are flanked at the 5′ end with XhoI and at the 3′ end with KpnI to facilitate direct cloning into a suitable binary vector for Agrobacterium-based plant transformation.

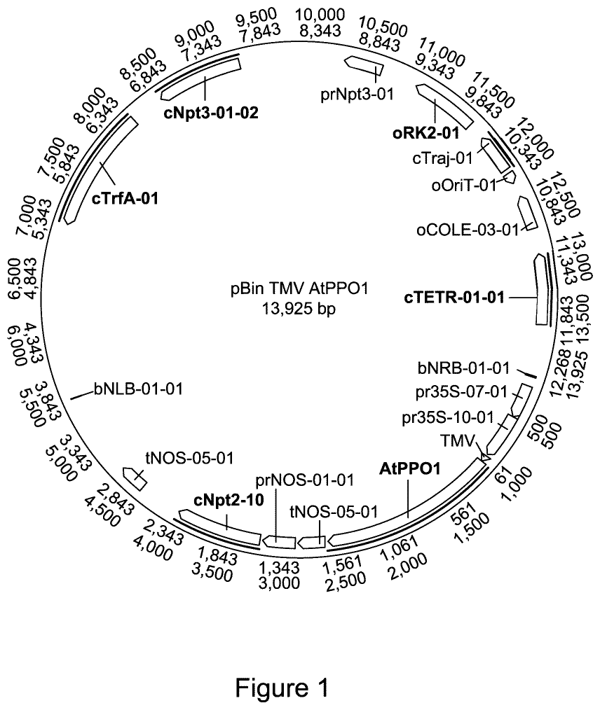

In one example, the expression cassette, comprising the TMV omega 5′ leader and a PPO1 encoding gene of interest is excised using XhoI/KpnI and cloned into similarly digested pBIN 19 (Bevan, Nucleic Acids Res. 12:8711-8721 (1984) to create a binary vector, e.g. the vector of FIG. 1, (SEQ ID NO: 152) behind a double enhanced 35S promoter ahead of a NOS 3′ transcription terminator and then transformed into E. coli DH5 alpha competent cells. DNA recovered from the E. coli is used to transform Agrobacterium tumefaciens LBA4404, and the transformed bacteria are selected on media contain rifampicin and kanamycin. Tobacco tissue is subjected to Agrobacterium-mediated transformation using methods well described in the art or as described herein. For example, a master plate of Agrobacterium tumefaciens containing the PPO1 expressing binary vector is used to inoculate 10 ml LB (L broth) containing 100 mg/l rifampicin plus 50 mg/l kanamycin using a single bacterial colony. This is incubated overnight at 28° C. shaking at 200 rpm. This entire overnight culture is used to inoculate a 50 ml volume of LB containing the same antibiotics. Again, this is cultured overnight at 28° C. shaking at 200 rpm. The Agrobacterium cells are pelleted by centrifuging at 3000 rpm for 15 minutes and then re-suspended in MS (Murashige and Skoog) medium containing 30 g/l sucrose, pH 5.9 to an OD (600 nM)=0.6. This suspension is dispensed in 25 ml aliquots into petri dishes.

Clonally micro-propagated tobacco shoot cultures are used to excise young (not yet fully expanded) leaves. The mid rib and outer leaf margins are removed and discarded, and the remaining lamina cut into 1 cm squares. These are transferred to the Agrobacterium suspension for 20 minutes. Explants are then removed, dabbed on sterile filter paper to remove excess suspension, then transferred onto solid NBM medium (MS medium containing 30 g/l sucrose, 1 mg/l BAP (benzylaminopurine) and 0.1 mg/l NAA (napthalene acetic acid) at pH 5.9 and solidified with 8 g/l Plantagar), with the abaxial surface of each explant in contact with the medium. Approximately 7 explants are transferred per plate, which are then sealed and maintained in a lit incubator at 25° C. for a 16 hour photoperiod for 3 days.

Explants are then transferred onto NBM medium containing 100 mg/l kanamycin plus antibiotics to prevent further growth of Agrobacterium (200 mg/l timentin with 250 mg/l carbenicillin). Further subculture onto this same medium was then performed every 2 weeks.

As shoots start to regenerate from the callusing leaf explants, these are removed to Shoot elongation medium (MS medium, 30 g/l sucrose, 8 g/l Plantagar, 100 mg/l kanamycin, 200 mg/l timentin, 250 mg/l carbenicillin, pH 5.9). Stable transgenic plants readily root within 2 weeks. To provide multiple plants per event to ultimately allow more than one herbicide test per transgenic plant, all rooting shoots are micropropagated to generate 3 or more rooted clones.

Putative transgenic plants that are rooting and showing vigorous shoot growth on the medium incorporating kanamycin are analysed by PCR using primers that amplified a 500 bp fragment specific to the AtPPO1 transgene of interest. Evaluation of this same primer set on untransformed tobacco showed conclusively that these primers would not amplify any sequences from the native tobacco genome.

Transformed shoots are divided into 2 or 3 clones and regenerated from kanamycin resistant callus. Shoots are rooted on MS agar containing kanamycin. Surviving rooted explants are re-rooted to provide approximately 40-50 kanamycin resistant and PCR positive events from each event.

Once rooted, plantlets are transferred from agar and potted into 50% peat, 50% John Innes Soil No. 3 with slow-release fertilizer in 3 inch round pots and left regularly watered to establish for 8-12d in the glass house. Glasshouse conditions are about 24-27° C. day; 18-21° C. night and approximately a 14 h photoperiod. Humidity is adjusted to ~65% and light levels used are up to 2000 mmol/m² at bench level.

Transgenic populations of about forty tobacco plants that comprise a gene encoding a full length Arabidopsis thaliana PPO1 gene (SEQ ID NO: 1) or variants (SEQ ID NO:1 and 124 to 137) or encoding a full length Setaria italica PPO1 gene (SEQ ID NO: 2) or variants (SEQ ID NO: 138-151) are thus produced. Plants are selected on the basis of similar size from each population and ELISA or Mass Western tests are carried out to monitor protein transgenic PPO expression levels. The highest expressing TO lines are selected to be taken forward to self and to generate T1 seed and T2 lines and seed in the normal way. Seeds from the highest expressing lines are tested for germination on agar plates containing a range of concentrations of PPO-inhibiting herbicides as taught for example herein and resistant plant lines selected as showing the least damage to root or leaf growth and morphology at the highest concentrations of herbicides. Resistant plant lines exhibit a dose response in respect of herbicidal damage by PPO1 inhibitors that is shifted to the right in comparison with similarly grown and treated wild type and null segregant plants.

### Example 5—Assay of Herbicide Tolerance in Transgenic Tobacco Plants Expressing Heterologous AtPPO1 Variants or Heterologous SiPPO1 Variants

In order to determine if the overexpression of the genes detailed in Example 3, the Setaria italica PPO1s (SEQ ID NO: 2 or 138-151) or any one of SEQ ID NO: 1, 2, 4-151, 155-302, or 305-336 confer resistance to PPO inhibiting compounds when expressed in plants, GM tobacco lines are produced and tested with PPO inhibiting compounds. Transgenic tobacco plants expressing the Arabidopsis thaliana PPO1 gene and variants of (SEQ ID NOs 1 and 124-137) are produced and transgenic tobacco plants expressing the Setaria italica PPO1 gene and variants of (SEQ ID NOs 2 and 138-151) are produced.

Populations of transgenic tobacco comprising 20-30 transgenic events per plant transformation constructs are generated as described in Example 4. These lines were clonally propagated and 1 clone per event was sprayed with PPO herbicides to confirm the herbicide resistance.

Herbicidal damage is visually assessed across the population and a herbicide damage score given at 7 and 14 days. A score of 0 indicates no visible damage or stunting whereas a score of 100 indicates a complete death of the plant. 20 transgenic events (i.e. individual transgenic plants) from each GM tobacco line are transplanted to soil 1 week prior to spraying with PPO inhibiting compounds. WT tobacco is included as a control. Table 8 below shows the treatment regimens used.

### Example 5A—Assay of Herbicide Tolerance in Transgenic Tobacco Plants Expressing Heterologous AtPPO1 Variants

Data for herbicide tolerance in transgenic tobacco plants was generated according to Example 5 above and is shown in Tables 11-24. Herbicidal damage was visually assessed across the population and a herbicide damage score given at 7 and 14 days. A score of 0 indicates no visible damage or stunting whereas a score of 100 indicates a complete death of the plant. 20 transgenic events (i.e. individual transgenic plants) from each GM tobacco line were transplanted to soil 1 week prior to spraying with PPO inhibiting compounds. WT tobacco is included as a control. Table 8 below shows the treatment regimens used.

TABLE 8
Treatment regimens for testing PPO inhibitor resistance

| Treatment | | Application | Formulation |
|---|---|---|---|
| no | Herbicide | rate (g ai/ha) | and Adjuvant |
| 1 | B | 120 | Genapol X080 |
| | | | 0.2% |
| 2 | C | 120 | Genapol X080 |
| | | | 0.2% |
| 3 | D | 120 | Genapol X080 |
| | | | 0.2% |

TABLE 9
Plants transformed with Arabidopsis PPO gene variants.

| | | Protein | DNA |
|---|---|---|---|
| | | SEQ | SEQ |
| Name | Mutations | ID NO | ID NO |
| pBin TMV AtPPO1 | Wild type | SEQ ID | SEQ ID |
| | | NO: 1 | NO: 3 |
| pBin TMV AtPPO1 V030 | S305L Y426V | SEQ ID | SEQ ID |
| | | NO: 125 | NO: 366 |
| pBin TMV AtPPO1 V004 | V365L Y426V L479N | SEQ ID | SEQ ID |
| | | NO: 126 | NO: 364 |
| pBin TMV AtPPO1 V036 | S305L G404A Y426V | SEQ ID | SEQ ID |
| | T431R L479N | NO: 127 | NO: 360 |
| pBin TMV AtPPO1 V055 | S305L V365M | SEQ ID | SEQ ID |
| | | NO: 128 | NO: 361 |
| pBin TMV AtPPO1 V018 | S305L Y362F G404A | SEQ ID | SEQ ID |
| | | NO: 129 | NO: 356 |
| pBin TMV AtPPO1 V020 | S305L Y361T V365L | SEQ ID | SEQ ID |
| | T431R L479M | NO: 130 | NO: 363 |
| pBin TMV AtPPO1 V002 | S305L Y426V L479M | SEQ ID | SEQ ID |
| | | NO: 131 | NO: 365 |
| pBin TMV AtPPO1 V025 | Y426M T461Q L479M | SEQ ID | SEQ ID |
| | | NO: 132 | NO: 358 |
| pBin TMV AtPPO1 V052 | S305L Y361D V365M | SEQ ID | SEQ ID |
| | | NO: 133 | NO: 368 |
| pBin TMV AtPPO1 V031 | S305L Y361C Y362F | SEQ ID | SEQ ID |
| | Y426M L479M | NO: 134 | NO: 367 |
| pBin TMV AtPPO1 V035 | Y361D V365L | SEQ ID | SEQ ID |
| | | NO: 135 | NO: 359 |
| pBin TMV AtPPO1 V022 | V365L G404A L479N | SEQ ID | SEQ ID |
| | | NO: 136 | NO: 357 |
| pBin TMV AtPPO1 V019 | S305L G404A L479N | SEQ ID | SEQ ID |
| | | NO: 137 | NO: 362 |

The results are shown in Tables 11 to 24 below. A control population of wild-type Samsun tobacco was assessed for herbicide damage with result shown in table 10. The wild-type Samsun plants show significant damage at the day 7 and 14 assessment time.

TABLE 10
Results of percentage of damage to WT tobacco plants

| | Wildtype Samsun tobacco | | | | | |
|---|---|---|---|---|---|---|
| WT | Herbicide C | | Herbicide D | | Herbicide B | |
| plant | 7 DAT | 14 DAT | 7 DAT | 14 DAT | 7 DAT | 14 DAT |
| 1 | 100 | 100 | 98 | 100 | 100 | 100 |
| 2 | 100 | 100 | 98 | 100 | 100 | 100 |
| 3 | 100 | 100 | 98 | 100 | 100 | 100 |
| 4 | 100 | 100 | 98 | 100 | 100 | 100 |
| 5 | 100 | 100 | 100 | 100 | 100 | 100 |
| 6 | 100 | 100 | 100 | 100 | 100 | 100 |
| 7 | 100 | 100 | 100 | 100 | 100 | 100 |
| 8 | 100 | 100 | 98 | 100 | 100 | 100 |

TABLE 11
Results of percentage of damage to tobacco
plants containing pBin TMV AtPPO1.

| | pBin TMV AtPPO1 | | | | | |
|---|---|---|---|---|---|---|
| Event | Herbicide C | | Herbicide D | | Herbicide B | |
| number | 7 DAT | 14 DAT | 7 DAT | 14 DAT | 7 DAT | 14 DAT |
| 0150 | 100 | 100 | 99 | 100 | 100 | 100 |
| 0154 | 100 | 100 | 100 | 100 | 100 | 100 |
| 0167 | 100 | 100 | 100 | 100 | 100 | 100 |
| 0169 | 95 | 90 | 95 | 95 | 99 | 100 |
| 0173 | 100 | 100 | 99 | 100 | 100 | 100 |
| 0177 | 95 | 100 | 97 | 99 | 99 | 100 |
| 0179 | 100 | 100 | 99 | 100 | 100 | 100 |
| 0184 | 100 | 100 | 99 | 100 | 100 | 100 |
| 0185 | 100 | 100 | 99 | 100 | 100 | 100 |
| 0188 | 100 | 100 | 100 | 100 | 100 | 100 |
| 0190 | 94 | 95 | 65 | 75 | 87 | 80 |
| 0193 | 95 | 97 | 95 | 100 | 100 | 100 |
| 0194 | 100 | 100 | 99 | 100 | 100 | 100 |
| 0195 | 100 | 100 | 100 | 100 | 100 | 100 |
| 0197 | 97 | 99 | 92 | 97 | 97 | 98 |
| 0215 | 100 | 100 | 99 | 100 | 100 | 100 |
| 0224 | 100 | 100 | 99 | 100 | 100 | 100 |
| 0225 | 97 | 100 | 92 | 96 | 99 | 99 |
| 0226 | 100 | 100 | 99 | 100 | 100 | 100 |
| 0231 | 100 | 100 | 98 | 99 | 100 | 100 |

TABLE 12
Results of percentage of damage to tobacco plants containing pBin TMV AtPPO1 V022
(Mutations V365L G404A L479N )

| | pBin TMV AtPPO1 V022 | | | | | |
|---|---|---|---|---|---|---|
| | Herbicide C | | Herbicide D | | Herbicide B | |
| | 7 DAT | 14 DAT | 7 DAT | 14 DAT | 7 DAT | 14 DAT |
| 9981 | 90 | 95 | 85 | 80 | 99 | 100 |
| 9984 | 99 | 100 | 90 | 95 | 98 | 100 |
| 9990 | 10 | 5 | 5 | 3 | 85 | 85 |
| 9992 | 100 | 100 | 99 | 100 | 100 | 100 |
| 0005 | 100 | 100 | 100 | 100 | 100 | 100 |
| 0008 | 100 | 100 | 99 | 100 | 100 | 100 |
| 0009 | 98 | 100 | 95 | 96 | 100 | 100 |
| 0016 | 98 | 100 | 95 | 100 | 99 | 100 |
| 0017 | 100 | 100 | 100 | 100 | 100 | 100 |
| 0018 | 100 | 100 | 98 | 100 | 100 | 100 |
| 0019 | 10 | 5 | 3 | 2 | 85 | 95 |
| 0026 | 100 | 100 | 99 | 100 | 100 | 100 |
| 0032 | 100 | 100 | 99 | 100 | 100 | 100 |
| 0033 | 65 | 60 | 20 | 5 | 80 | 70 |
| 0045 | 7 | 3 | 5 | 3 | 75 | 70 |
| 0052 | 99 | 100 | 99 | 100 | 99 | 100 |
| 0054 | 5 | 2 | 3 | 0 | 90 | 95 |
| 0055 | 99 | 100 | 99 | 100 | 100 | 100 |
| 0062 | 100 | 100 | 100 | 100 | 100 | 100 |
| 0063 | 100 | 100 | 99 | 100 | 100 | 100 |

TABLE 13
Results of percentage of damage to tobacco plants containing pBin TMV AtPPO1 V025
(mutations Y426M T461Q L479M)

| | pBin TMV AtPPO1 V025 | | | | | |
|---|---|---|---|---|---|---|
| | Herbicide C | | Herbicide D | | Herbicide B | |
| | 7 DAT | 14 DAT | 7 DAT | 14 DAT | 7 DAT | 14 DAT |
| 0319 | 65 | 45 | 55 | 30 | 99 | 100 |
| 0324 | 0 | 0 | 0 | 0 | 85 | 80 |
| 0326 | 100 | 100 | 100 | 100 | 100 | 100 |
| 0331 | 95 | 97 | 90 | 85 | 100 | 100 |
| 0337 | 85 | 90 | 75 | 75 | 99 | 100 |
| 0341 | 85 | 80 | 85 | 70 | 100 | 100 |
| 0346 | 10 | 5 | 35 | 5 | 95 | 98 |
| 0351 | 35 | 20 | 35 | 5 | 97 | 100 |
| 0356 | 96 | 98 | 95 | 97 | 99 | 100 |
| 0359 | 99 | 100 | 100 | 100 | 100 | 100 |
| 0364 | 100 | 100 | 100 | 100 | 100 | 100 |
| 0367 | 98 | 100 | 95 | 99 | 100 | 100 |
| 0370 | 10 | 0 | 10 | 5 | 99 | 100 |
| 0373 | 35 | 10 | 50 | 15 | 97 | 100 |
| 0387 | 75 | 95 | 75 | 95 | 99 | 100 |
| 0390 | 100 | 100 | 100 | 100 | 100 | 100 |
| 0392 | 0 | 0 | 1 | 0 | 98 | 95 |
| 0397 | 35 | 20 | 70 | 45 | 99 | 100 |
| 0398 | 20 | 15 | 40 | 30 | 99 | 100 |
| 0400 | 50 | 20 | 35 | 15 | 99 | 100 |

TABLE 14
Results of percentage of damage to tobacco plants containing pBin TMV AtPPO1 V035.
(Mutations Y361D V365L)

| | pBin TMV AtPPO1 V035 | | | | | |
|---|---|---|---|---|---|---|
| | Herbicide C | | Herbicide D | | Herbicide B | |
| | 7 DAT | 14 DAT | 7 DAT | 14 DAT | 7 DAT | 14 DAT |
| 0245 | 98 | 100 | 99 | 100 | 100 | 100 |
| 0250 | 98 | 100 | 99 | 100 | 100 | 100 |
| 0251 | 50 | 10 | 80 | 75 | 99 | 100 |
| 0252 | 100 | 100 | 99 | 100 | 100 | 100 |
| 0254 | 100 | 100 | 100 | 100 | 100 | 100 |
| 0258 | 99 | 100 | 99 | 100 | 100 | 100 |
| 0260 | 45 | 40 | 80 | 80 | 100 | 100 |
| 0264 | 100 | 100 | 99 | 100 | 100 | 100 |
| 0267 | 100 | 100 | 100 | 100 | 100 | 100 |
| 0268 | 99 | 100 | 99 | 100 | 100 | 100 |
| 0271 | 99 | 100 | 99 | 99 | 100 | 100 |
| 0273 | 99 | 100 | 99 | 100 | 99 | 100 |
| 0276 | 80 | 85 | 85 | 85 | 99 | 100 |
| 0286 | 80 | 80 | 90 | 85 | 99 | 100 |
| 0287 | 100 | 100 | 97 | 99 | 99 | 100 |
| 0292 | 60 | 35 | 75 | 60 | 85 | 70 |
| 0298 | 80 | 80 | 80 | 80 | 100 | 100 |
| 0306 | 95 | 96 | 90 | 95 | 99 | 100 |
| 0310 | 100 | 100 | 100 | 100 | 100 | 100 |
| 0313 | 30 | 30 | 50 | 70 | 97 | 95 |

TABLE 15
Results of percentage of damage to tobacco plants containing pBin TMV AtPPO1 V055.
(Mutations S305L V365M)

| | pBin TMV AtPPO1 V055 | | | | | |
|---|---|---|---|---|---|---|
| | Herbicide C | | Herbicide D | | Herbicide B | |
| | 7 DAT | 14 DAT | 7 DAT | 14 DAT | 7 DAT | 14 DAT |
| 9897 | 65 | 60 | 90 | 95 | 99 | 100 |
| 9899 | 95 | 98 | 99 | 100 | 100 | 100 |
| 9902 | 25 | 5 | 30 | 5 | 99 | 100 |
| 9909 | 10 | 5 | 30 | 5 | 90 | 70 |
| 9918 | 55 | 40 | 50 | 55 | 99 | 100 |
| 9923 | 35 | 10 | 45 | 25 | 99 | 100 |
| 9927 | 100 | 100 | 100 | 100 | 100 | 100 |
| 9931 | 5 | 5 | 20 | 8 | 90 | 80 |
| 9935 | 99 | 100 | 99 | 100 | 100 | 100 |
| 9936 | 97 | 99 | 99 | 100 | 98 | 99 |
| 9938 | 95 | 95 | 96 | 97 | 100 | 100 |
| 9942 | 20 | 5 | 85 | 70 | 98 | 99 |
| 9943 | 35 | 30 | 80 | 85 | 92 | 85 |
| 9947 | 90 | 96 | 96 | 99 | 99 | 100 |
| 9953 | 6 | 5 | 25 | 10 | 99 | 100 |
| 9955 | 20 | 8 | 25 | 30 | 95 | 95 |
| 9959 | 100 | 100 | 100 | 100 | 100 | 100 |
| 9962 | 10 | 5 | 30 | 8 | 85 | 50 |
| 9973 | 100 | 100 | 100 | 100 | 100 | 100 |
| 9977 | 5 | 3 | 30 | 10 | 98 | 95 |

TABLE 16
Results of percentage of damage to tobacco plants containing pBin TMV AtPPO1 V019.
(Mutations S305L G404A L479N)

| | pBin TMV AtPPO1 V019 | | | | | |
|---|---|---|---|---|---|---|
| | Herbicide C | | Herbicide D | | Herbicide B | |
| | 7 DAT | 14 DAT | 7 DAT | 14 DAT | 7 DAT | 14 DAT |
| 0067 | 35 | 30 | 15 | 25 | 75 | 50 |
| 0072 | 65 | 60 | 15 | 20 | 80 | 65 |
| 0080 | 75 | 90 | 30 | 40 | 80 | 80 |
| 0086 | 65 | 55 | 30 | 25 | 90 | 85 |
| 0096 | 75 | 75 | 30 | 10 | 80 | 85 |
| 0097 | 55 | 25 | 20 | 5 | 60 | 30 |
| 0102 | 55 | 20 | 25 | 2 | 85 | 90 |
| 0103 | 75 | 85 | 25 | 25 | 100 | 100 |
| 0104 | 85 | 95 | 60 | 80 | 98 | 100 |
| 0105 | 40 | 30 | 20 | 15 | 75 | 55 |
| 0106 | 55 | 50 | 25 | 20 | 80 | 50 |
| 0107 | 100 | 100 | 100 | 100 | 100 | 100 |
| 0108 | 97 | 100 | 95 | 95 | 97 | 100 |
| 0115 | 55 | 50 | 40 | 30 | 85 | 75 |
| 0126 | 100 | 100 | 99 | 100 | 100 | 100 |
| 0131 | 70 | 70 | 35 | 35 | 80 | 55 |
| 0137 | 97 | 100 | 95 | 96 | 99 | 100 |
| 0140 | 100 | 100 | 100 | 100 | 100 | 100 |
| 0141 | 100 | 100 | 99 | 100 | 100 | 100 |
| 0143 | 70 | 85 | 35 | 30 | 85 | 75 |

TABLE 17
Results of percentage of damage to tobacco plants containing pBin TMV AtPPO1 V020.
(Mutations S305L Y361T V365L T431R L479M)

| | pBin TMV AtPPO1 V020 | | | | | |
|---|---|---|---|---|---|---|
| | Herbicide C | | Herbicide D | | Herbicide B | |
| | 7 DAT | 14 DAT | 7 DAT | 14 DAT | 7 DAT | 14 DAT |
| 0407 | 100 | 100 | 97 | 100 | 100 | 100 |
| 0411 | 95 | 99 | 95 | 98 | 99 | 100 |
| 0414 | 90 | 95 | 90 | 85 | 99 | 100 |
| 0420 | 100 | 100 | 100 | 100 | 100 | 100 |
| 0425 | 100 | 100 | 100 | 100 | 100 | 100 |
| 0428 | 100 | 100 | 99 | 100 | 100 | 100 |
| 0430 | 100 | 100 | 99 | 100 | 100 | 100 |
| 0433 | 99 | 100 | 99 | 100 | 100 | 100 |
| 0438 | 99 | 100 | 97 | 100 | 100 | 100 |
| 0442 | 85 | 80 | 85 | 90 | 99 | 100 |
| 0443 | 100 | 100 | 100 | 100 | 100 | 100 |
| 0444 | 100 | 100 | 100 | 100 | 100 | 100 |
| 0447 | 75 | 80 | 75 | 80 | 92 | 90 |
| 0449 | 90 | 97 | 90 | 96 | 99 | 99 |
| 0451 | 100 | 100 | 99 | 100 | 100 | 100 |
| 0460 | 95 | 95 | 90 | 90 | 99 | 100 |
| 0471 | 99 | 100 | 99 | 100 | 100 | 100 |
| 0478 | 95 | 96 | 90 | 90 | 100 | 100 |
| 0484 | 100 | 100 | 100 | 100 | 100 | 100 |
| 0485 | 100 | 100 | 100 | 100 | 100 | 100 |

TABLE 18
Results of percentage of damage to tobacco plants containing pBin TMV AtPPO1 V004.
(Mutations V365L Y426V L479N)

| | pBin TMV AtPPO1 V004 | | | | | |
|---|---|---|---|---|---|---|
| | Herbicide C | | Herbicide D | | Herbicide B | |
| | 7 DAT | 14 DAT | 7 DAT | 14 DAT | 7 DAT | 14 DAT |
| 9567 | 100 | 100 | 100 | 100 | 100 | 100 |
| 9568 | 25 | 25 | 10 | 15 | 85 | 75 |
| 9571 | 0 | 0 | 0 | 0 | 80 | 65 |
| 9575 | 0 | 0 | 0 | 0 | 90 | 65 |
| 9577 | 30 | 25 | 20 | 0 | 99 | 100 |
| 9578 | 0 | 0 | 0 | 0 | 68 | 50 |
| 9580 | 65 | 70 | 70 | 60 | 99 | 100 |
| 9582 | 0 | 0 | 0 | 0 | 30 | 15 |
| 9585 | 85 | 90 | 85 | 85 | 100 | 100 |
| 9587 | 100 | 100 | 100 | 100 | 100 | 100 |
| 9588 | 15 | 0 | 20 | 5 | 97 | 95 |
| 9592 | 80 | 85 | 85 | 75 | 100 | 100 |
| 9599 | 5 | 0 | 3 | 0 | 85 | 65 |
| 9603 | 0 | 0 | 5 | 5 | 70 | 25 |
| 9604 | 0 | 0 | 0 | 0 | 99 | 100 |
| 9608 | 0 | 0 | 0 | 0 | 90 | 75 |
| 9625 | 98 | 100 | 96 | 96 | 100 | 100 |
| 9626 | 20 | 10 | 20 | 15 | 99 | 100 |
| 9631 | 100 | 100 | 100 | 100 | 100 | 100 |
| 9634 | 40 | 20 | 45 | 10 | 99 | 100 |

TABLE 19
Results of percentage of damage to tobacco plants containing pBin TMV AtPPO1 V036.
(Mutations S305L G404A Y426V T431R L479N)

| | pBin TMV AtPPO1 V036 | | | | | |
|---|---|---|---|---|---|---|
| | Herbicide C | | Herbicide D | | Herbicide B | |
| | 7 DAT | 14 DAT | 7 DAT | 14 DAT | 7 DAT | 14 DAT |
| 9654 | 65 | 70 | 65 | 75 | 75 | 75 |
| 9655 | 100 | 100 | 100 | 100 | 100 | 100 |
| 9660 | 100 | 100 | 100 | 100 | 100 | 100 |
| 9668 | 65 | 75 | 70 | 75 | 75 | 70 |
| 9670 | 65 | 75 | 60 | 70 | 65 | 65 |
| 9671 | 55 | 70 | 55 | 50 | 65 | 50 |
| 9679 | 100 | 100 | 100 | 100 | 100 | 100 |
| 9681 | 50 | 55 | 55 | 65 | 60 | 50 |
| 9683 | 70 | 90 | 75 | 80 | 85 | 98 |
| 9684 | 100 | 100 | 100 | 100 | 100 | 100 |
| 9686 | 100 | 100 | 100 | 100 | 100 | 100 |
| 9688 | 98 | 100 | 95 | 99 | 96 | 100 |
| 9692 | 65 | 75 | 65 | 70 | 75 | 70 |
| 9694 | 70 | 70 | 75 | 70 | 70 | 60 |
| 9697 | 100 | 100 | 100 | 100 | 100 | 100 |
| 9698 | 100 | 100 | 100 | 100 | 100 | 100 |
| 9703 | 60 | 45 | 94 | 95 | 85 | 85 |
| 9709 | 50 | 10 | 50 | 15 | 40 | 5 |
| 9712 | 100 | 100 | 100 | 100 | 100 | 100 |
| 9717 | 40 | 30 | 35 | 20 | 35 | 20 |

TABLE 20
Results of percentage of damage to tobacco plants containing pBin TMV AtPPO1 V002.
(mutations S305L Y426V L479M )

| | Bin TMV AtPPO1 V002 | | | | | |
|---|---|---|---|---|---|---|
| | Herbicide C | | Herbicide D | | Herbicide B | |
| | 7 DAT | 14 DAT | 7 DAT | 14 DAT | 7 DAT | 14 DAT |
| 9898 | 90 | 75 | 30 | 10 | 100 | 100 |
| 9901 | 80 | 75 | 30 | 10 | 100 | 100 |
| 9903 | 0 | 0 | 10 | 20 | 15 | 10 |
| 9907 | 15 | 5 | 5 | 0 | 100 | 100 |
| 9910 | 25 | 10 | 5 | 0 | 99 | 100 |
| 9911 | 0 | 2 | 0 | 0 | 85 | 65 |
| 9914 | 100 | 100 | 100 | 100 | 100 | 100 |
| 9918 | 100 | 100 | 100 | 100 | 100 | 100 |
| 9919 | 90 | 85 | 65 | 25 | 100 | 100 |
| 9922 | 96 | 100 | 85 | 90 | 100 | 100 |
| 9923 | 98 | 100 | 93 | 97 | 100 | 100 |
| 9924 | 0 | 0 | 0 | 0 | 25 | 20 |
| 9928 | 0 | 0 | 0 | 0 | 95 | 92 |
| 9929 | 100 | 100 | 100 | 100 | 100 | 100 |
| 9930 | 5 | 0 | 5 | 0 | 85 | 65 |
| 9939 | 0 | 0 | 0 | 0 | 50 | 35 |
| 9940 | 0 | 10 | 0 | 5 | 60 | 55 |
| 9941 | 0 | 0 | 0 | 0 | 70 | 50 |
| 9943 | 98 | 96 | 80 | 90 | 100 | 100 |
| 9953 | 100 | 100 | 100 | 100 | 100 | 100 |

TABLE 21
Results of percentage of damage to tobacco plants containing pBin TMV AtPPO1 V030.
(Mutations S305L Y426V)

| | pBin TMV AtPPO1 V030 | | | | | |
|---|---|---|---|---|---|---|
| | Herbicide C | | Herbicide D | | Herbicide B | |
| | 7 DAT | 14 DAT | 7 DAT | 14 DAT | 7 DAT | 14 DAT |
| 9816 | 85 | 70 | 45 | 40 | 100 | 100 |
| 9818 | 0 | 0 | 0 | 0 | 95 | 85 |
| 9821 | 25 | 0 | 10 | 0 | 85 | 50 |
| 9823 | 0 | 0 | 8 | 2 | 85 | 65 |
| 9826 | 10 | 10 | 15 | 5 | 99 | 100 |
| 9829 | 90 | 95 | 85 | 85 | 99 | 100 |
| 9830 | 0 | 0 | 0 | 0 | 75 | 25 |
| 9836 | 96 | 97 | 85 | 85 | 99 | 100 |
| 9838 | 100 | 100 | 100 | 100 | 100 | 100 |
| 9839 | 5 | 0 | 0 | 0 | 95 | 90 |
| 9840 | 90 | 90 | 80 | 75 | 100 | 100 |
| 9843 | 0 | 0 | 0 | 0 | 85 | 75 |
| 9851 | 100 | 100 | 100 | 100 | 100 | 100 |
| 9855 | 0 | 0 | 0 | 0 | 85 | 70 |
| 9868 | 2 | 0 | 0 | 0 | 85 | 80 |
| 9871 | 40 | 55 | 30 | 5 | 100 | 100 |
| 9885 | 98 | 100 | 90 | 90 | 100 | 100 |
| 9890 | 0 | 0 | 0 | 0 | 75 | 50 |
| 9893 | 0 | 0 | 0 | 0 | 80 | 50 |
| 9894 | 0 | 0 | 0 | 0 | 85 | 65 |

TABLE 22
Results of percentage of damage to tobacco plants containing pBin TMV AtPPO1 V052.
(Mutations S305L Y361D V365M)

| | pBin TMV AtPPO1 V052 | | | | | |
|---|---|---|---|---|---|---|
| | Herbicide C | | Herbicide D | | Herbicide B | |
| D4562 | 7 DAT | 14 DAT | 7 DAT | 14 DAT | 7 DAT | 14 DAT |
| 9732 | 65 | 80 | 92 | 85 | 100 | 100 |
| 9743 | 99 | 100 | 100 | 100 | 100 | 100 |
| 9744 | 60 | 70 | 90 | 90 | 98 | 100 |
| 9754 | 100 | 100 | 100 | 100 | 100 | 100 |
| 9755 | 99 | 100 | 99 | 100 | 100 | 100 |
| 9756 | 60 | 35 | 80 | 70 | 96 | 97 |
| 9758 | 80 | 45 | 90 | 75 | 100 | 100 |
| 9761 | 75 | 35 | 85 | 55 | 100 | 100 |
| 9774 | 75 | 30 | 90 | 70 | 99 | 100 |
| 9778 | 100 | 100 | 100 | 100 | 100 | 100 |
| 9779 | 99 | 100 | 100 | 100 | 100 | 100 |
| 9793 | 95 | 90 | 97 | 95 | 100 | 100 |
| 9795 | 80 | 50 | 90 | 70 | 100 | 100 |
| 9801 | 100 | 100 | 100 | 100 | 100 | 100 |
| 9802 | 100 | 100 | 100 | 100 | 100 | 100 |
| 9805 | 96 | 96 | 96 | 97 | 99 | 100 |
| 9807 | 100 | 100 | 100 | 100 | 100 | 100 |
| 9808 | 97 | 98 | 99 | 100 | 100 | 100 |
| 9809 | 90 | 70 | 85 | 60 | 100 | 100 |
| 9812 | 100 | 100 | 99 | 100 | 100 | 100 |

TABLE 23
Results of percentage of damage to tobacco plants containing pBin TMV AtPPO1 V031.
(Mutations S305L Y361C Y362F Y426M L479M)

| | pBin TMV AtPPO1 V031 | | | | | |
|---|---|---|---|---|---|---|
| | Herbicide C | | Herbicide D | | Herbicide B | |
| | 7 DAT | 14 DAT | 7 DAT | 14 DAT | 7 DAT | 14 DAT |
| 0487 | 60 | 45 | 50 | 30 | 100 | 100 |
| 0490 | 0 | 0 | 0 | 0 | 96 | 90 |
| 0491 | 95 | 98 | 98 | 100 | 100 | 100 |
| 0495 | 100 | 100 | 98 | 100 | 100 | 100 |
| 0500 | 85 | 95 | 95 | 95 | 100 | 100 |
| 0507 | 5 | 0 | 5 | 5 | 85 | 70 |
| 0509 | 100 | 100 | 98 | 100 | 100 | 100 |
| 0518 | 0 | 0 | 0 | 5 | 96 | 100 |
| 0520 | 0 | 0 | 0 | 0 | 95 | 100 |
| 0521 | 0 | 0 | 0 | 0 | 95 | 90 |
| 0522 | 0 | 0 | 0 | 0 | 75 | 60 |
| 0527 | 100 | 100 | 98 | 100 | 100 | 100 |
| 0535 | 0 | 0 | 0 | 0 | 85 | 75 |
| 0540 | 100 | 100 | 98 | 100 | 100 | 100 |
| 0543 | 100 | 100 | 98 | 100 | 100 | 100 |
| 0551 | 90 | 90 | 95 | 90 | 100 | 100 |
| 0553 | 0 | 0 | 0 | 0 | 80 | 85 |
| 0557 | 0 | 0 | 0 | 0 | 80 | 75 |
| 0559 | 0 | 0 | 0 | 0 | 70 | 40 |
| 0561 | 100 | 100 | 98 | 100 | 100 | 100 |

TABLE 24
Results of percentage of damage to tobacco plants containing pBin TMV AtPPO1 V018.
(mutations S305L Y362F G404A)

| | pBin TMV AtPPO1 V018 | | | | | |
|---|---|---|---|---|---|---|
| | Herbicide C | | Herbicide D | | Herbicide B | |
| | 7 DAT | 14 DAT | 7 DAT | 14 DAT | 7 DAT | 14 DAT |
| 0571 | 80 | 85 | 80 | 70 | 90 | 95 |
| 0573 | 60 | 70 | 80 | 90 | 85 | 80 |
| 0575 | 100 | 100 | 95 | 100 | 100 | 100 |
| 0576 | 100 | 100 | 100 | 100 | 100 | 100 |
| 0579 | 95 | 100 | 90 | 95 | 98 | 100 |
| 0582 | 85 | 90 | 90 | 90 | 95 | 98 |
| 0587 | 100 | 100 | 98 | 100 | 100 | 100 |
| 0593 | 100 | 100 | 98 | 100 | 100 | 100 |
| 0595 | 100 | 100 | 98 | 100 | 100 | 100 |
| 0598 | 100 | 100 | 100 | 100 | 100 | 100 |
| 0600 | 100 | 100 | 100 | 100 | 100 | 100 |
| 0605 | 100 | 100 | 98 | 100 | 100 | 100 |
| 0611 | 100 | 100 | 98 | 100 | 100 | 100 |
| 0612 | 50 | 75 | 35 | 30 | 80 | 85 |
| 0617 | 100 | 100 | 98 | 100 | 100 | 100 |
| 0618 | 75 | 80 | 60 | 65 | 97 | 100 |
| 0620 | 100 | 100 | 98 | 100 | 100 | 100 |
| 0624 | 100 | 100 | 98 | 100 | 100 | 100 |
| 0628 | 100 | 100 | 98 | 100 | 100 | 100 |
| 0644 | 100 | 100 | 98 | 100 | 100 | 100 |

### Example 6: Transformation of Corn with AtPPO1 or SiPPO1 Variants

Transgenic corn lines expressing AtPPO1 and variants (SEQ ID NO:1 and 124-137) and SiPPO1 and variants (SEQ ID NO:2 and 138-151) are created using methods known in the art.

Transformation of immature maize embryos is performed essentially as described in Negrotto et al. (Plant Cell Reports (2000) 19: 798-803). Briefly, Agrobacterium strain LBA4404 (pSB1) comprising an expression vector expressing the disclosed AtPPO1 and variants (SEQ ID NO: 1 and 124-137) and SiPPO1 and variants (SEQ ID NO: 2 and 138-151) is grown on YEP (yeast extract (5 g/L), peptone (10 g/L), NaCl (5 g/L), 15 g/l agar, pH 6.8) solid medium for 2-4 days at 28° C. Approximately 0.8×10⁹ Agrobacterium cells are suspended in LS-inf media supplemented with 100 μM As. Bacteria are pre-induced in this medium for approximately 30-60 minutes.

Immature embryos from an inbred maize line are excised from 8-12 day old ears into liquid LS-inf+100 μM As. Embryos are rinsed once with fresh infection medium. Agrobacterium solution is then added, and embryos are vortexed for 30 seconds and allowed to settle with the bacteria for 5 minutes. The embryos are then transferred scutellum side up to LSAs medium and cultured in the dark for two to three days. Subsequently, between approximately 20 and 25 embryos per petri plate are transferred to LSDc medium supplemented with cefotaxime (250 mg/l) and silver nitrate (1.6 mg/l) and cultured in the dark at approximately 28° C. for 10 days.

Immature embryos, producing embryogenic callus are transferred to LSD1M0.5S medium. The cultures are selected on this medium for approximately 6 weeks with a subculture step at about 3 weeks. Surviving calli are transferred to Reg1 medium supplemented with mannose. Following culturing in the light (16 hour light/8 hour dark regiment), green tissues are then transferred to Reg2 medium without growth regulators and incubated for about 1-2 weeks. Plantlets are transferred to Magenta GA-7 boxes (Magenta Corp, Chicago Ill.) containing Reg3 medium and grown in the light. After about 2-3 weeks, plants are tested for the presence of the disclosed PPO genes by PCR. Positive plants from the PCR assay are transferred to a greenhouse for further evaluation.

Plants are treated with PPO inhibiting herbicides to confirm the herbicide resistance.

### Example 7: Transformation of Soybean with AtPPO1 or SiPPO1 Variants

Transgenic soybean lines expressing AtPPO1 and variants (SEQ ID NO:1 and 124-137) and SiPPO1 and variants (SEQ ID NO:2 and 138-151) are created.

Soybean plant material can be suitably transformed and fertile plants regenerated by many methods which are well known to one of skill in the art. For example, fertile morphologically normal transgenic soybean plants may be obtained by: 1) production of somatic embryogenic tissue from, e.g., immature cotyledon, hypocotyl or other suitable tissue; 2) transformation by particle bombardment or infection with Agrobacterium; and 3) regeneration of plants. In one example, as described in U.S. Pat. No. 5,024,944, cotyledon tissue is excised from immature embryos of soybean, optionally with the embryonic axis removed, and cultured on hormone-containing medium so as to form somatic embryogenic plant material. This material is transformed using, for example, direct DNA methods, DNA coated microprojectile bombardment or infection with Agrobacterium, cultured on a suitable selection medium and regenerated, optionally also in the continued presence of selecting agent, into fertile transgenic soybean plants. Selection agents may be antibiotics such as kanamycin, hygromycin, or herbicides or, alternatively, selection may be based upon expression of a visualisable marker gene such as GUS. Target tissues for transformation include meristematic tissue, somaclonal embryogenic tissue, and flower or flower-forming tissue. Other examples of soybean transformation include physical DNA delivery methods, such as particle bombardment (see e.g., Finer & McMullen, In Vitro Cell Dev. Biol, 1991, 27P: 175-182; McCabe et al, Bio/technology, 1998, 6:923-926), whisker (Khalafalla et al, African J. of Biotechnology, 2006, 5: 1594-1599), aerosol bean injection (U.S. Pat. No. 7,001,754), or by Agrobacterium-mediated delivery methods (Hinchee et al, Bio/Technology, 1988, 6:915-922; U.S. Pat. No. 7,002,058; U.S. Patent Application Publication Nos. 20040034889 and 20080229447; Paz et al, Plant Cell Report, 2006, 25:206-213).

Soybean transgenic plants can be generated with a binary vector containing AtPPO1 and variants (SEQ ID NO:1 and 124-137) or SiPPO1 and variants (SEQ ID NO:2 and 138-151) using any available transformation method. Optionally, the PPO gene can provide the means of selection and identification of transgenic tissue. For example, a vector is used to transform immature seed targets as described to generate transgenic PPO soybean plants directly using PPO inhibitor, as selection agent. A PPO gene can be present in the polynucleotide alongside other sequences which provide additional means of selection/identification of transformed tissue including, for example, the known genes which provide resistance to kanamycin, hygromycin, phosphinothricin, butafenacil, or glyphosate. For example, different binary vectors containing PAT or EPSPS selectable marker genes are known in the art (see e.g., U.S. Patent Application Publication No. 20080229447). Alternatively, selectable marker sequences may be present on separate polynucleotides and a process of, for example, co-transformation and co-selection is used. A scorable marker gene such as GUS may also be used to identify transformed tissue.

TO plants are taken from tissue culture to the greenhouse where they are transplanted into water-saturated soil (REDI-EARTH® Plug and Seedling Mix, Sun Gro Horticulture, Bellevue, WA, or Fafard Germinating Mix) mixed with 1% granular MARATHON® (Olympic Horticultural Products, Co., Mainland, PA) at 5-10 g/gal soil in 2″ square pots. The plants are covered with humidity domes and placed in a Conviron chamber (Pembina, ND) with the following environmental conditions: 24° C. day; 20° C. night; 16-23 hours light-1-8 hours dark photoperiod; 80% relative humidity.

After plants are established in the soil and new growth appeared (−1-2 weeks), plants are sampled and tested for the presence of desired transgene by TAQMAN® analysis using appropriate probes for the PPO genes or promoters. Positive plants are transplanted into 4″ square pots containing Fafard #3 soil. Sierra 17-6-12 slow release fertilizer is incorporated into the soil at the recommended rate. The plants are then relocated into a standard greenhouse to acclimatize (~1 week). The environmental conditions are: 27° C. day; 21° C. night; 14 hour photoperiod (with supplemental light); ambient humidity. After acclimatizing (~1 week), the plants are sampled and tested in detail for the presence and copy number of inserted transgenes. Transgenic soybean plants are grown to maturity for TI seed production.

TI plants are grown up, and are tested for the presence of the disclosed PPO genes by PCR. Positive plants from the PCR assay are transferred to a greenhouse for further evaluation. After TAQMAN® analysis, homozygous plants were grown for seed production. Transgenic seeds and progeny plants are used to further evaluate their herbicide tolerance performance and molecular characteristics

Plants are treated with PPO inhibiting herbicides to confirm the herbicide resistance.

### Example 8: Generating Transgenic Corn and Soy Plants Expressing Heterologous AtPPO1 and SiPPO1 Variants

The transformation methods described in Examples 6 and 7 were used to transform corn and soy with AtPPO1 and SiPPO1 variants disclosed herein.

At least 6 events/PPO1 variant shown in Table 25 and Table 26 were selected to advance to T1 screening. For each event, 36-54 seeds per event were sown. At 10 days after sowing, leaf tissue of all seeds that successfully germinated was sampled for TAQMAN® as noted in Example 7 and zygosity determined. Chi-square analyses were performed on all zygosity data to determine segregation pattern of each event. The Chi-square null hypothesis was set to a 1:2:1 ratio of Null:Heterozygous:Homozygous. A p-value cut-off of <=0.05 was used to reject the null hypothesis.

TABLE 25
PPO1 variants expressed in corn

AtPPO1-V0
AtPPO1-V2
AtPPO1-V4
AtPPO1-V18
AtPPO1-V19
AtPPO1-V22
AtPPO1-V25
AtPPO1-V30
AtPPO1-V31
AtPPO1-V35
AtPPO1-V36
AtPPO1-V52
AtPPO1-V55
SiPPO1-V4
SiPPO1-V18
SiPPO1-V30
SiPPO1-V55

TABLE 26
PPO1 variants expressed in soy

AtPPO1-V0
AtPPO1-V2
AtPPO1-V4
AtPPO1-V18
AtPPO1-V19
AtPPO1-V20
AtPPO1-V22
AtPPO1-V25
AtPPO1-V30
AtPPO1-V31
AtPPO1-V35
AtPPO1-V36
AtPPO1-V52
AtPPO1-V55
SiPPO1-V0
SiPPO1-V2
SiPPO1-V4
SiPPO1-V18
SiPPO1-V30
SiPPO1-V36
SiPPO1-V55

### TABLES OF DETAILED DESCRIPTION

TABLE 27
Modified PPO motifs

| Modified | | |
|---|---|---|
| residue | | |
| (with | | |
| reference | | SEQ |
| to SEQ ID | | ID |
| NO: 1) | Motif (modified residue shown in bold) | NO |
| S305 | S; (K/N/R); (L/V); K; L; S; W; (E/K/T); L; L; | 337 |
| | (G/S); I; (I/K/S/T); K | |
| S305-2 | K; L; S; W; (E/K/T); L; L; (G/S); I | 346 |
| Y361 | L; (A/S); (I/K/N/Q/R/S); (F/L); (D/F/P/Q/R/T); Y; | 338 |
| | P; P; V; (A/G); (A/S); V | |
| Y361-2 | (D/F/P/Q/R/T); Y; P; P; V; (A/G); (A/S); V | 347 |
| Y362 | L; (A/S); (I/K/N/Q/R/S); (F/L); (D/F/H/Y); F; P; P; | 339 |
| | V; (A/G); (A/S); V | |
| Y362-2 | (F/L); (D/F/H/Y); F; P; P; V; (A/G); (A/S); V | 348 |
| V365 | L; (A/S); (I/K/N/Q/R/S); (F/L); (D/F/H/Y); Y; P; P; | 340 |
| | (L/M); (A/G); (A/S); V | |
| V365-2 | Y; P; P; (L/M); (A/G); (A/S); V | 349 |
| G404 | G; (I/L/V); E; T; L; A; (S/T); I; Y; S; S; S; L; F; P | 341 |
| G404-2 | E; T; L; A; (S/T); I; Y; S; S; S; L; F; P | 350 |
| Y426 | L; F; P; (G/N); R; A; P; (A/D/K/N/P/S); G; (Q/R); | 342 |
| | (I/T/V); (M/L); (I/L); L; (N/S); (C/L/M/T/V); I; G; | |
| | G; (A/S); (K/L/T); N | |
| Y426-2 | L; (N/S); (C/L/M/T/V); I; G; G; (A/S); (K/L/T); N | 351 |
| T431 | L; F; P; (G/N); R; A; P; (A/D/K/N/P/S); G; (Q/R); | 343 |
| | (I/T/V); (M/L); (I/L); L; (N/S); (F/Y); I; G; G; | |
| | (A/S); R; N | |
| T431-2 | L; (N/S); (F/Y); I; G; G; (A/S); R; N | 352 |
| T461 | (G/P/S); (D/K/N/R/S/T); (A/S); Q; (D/E); P; | 344 |
| | (F/L/R/S); (A/T/V); (L/T/V); (G/S); V; (K/R); V; | |
| | W; (P/R); (K/Q/R); A; I; P; Q; F; L; (I/V); G | |
| T461-2 | A/S); Q; (D/E); P; (F/L/R/S); (A/T/V); (L/T/V); | 353 |
| | (G/S); V; (K/R); V; W; (P/R); (K/Q/R); A; I; P; Q; | |
| | F; L | |
| L479 | (G/P/S); (D/K/N/R/S/T); (A/S); (A/E/G/K/Q/V); | 345 |
| | (D/E); P; (F/L/R/S); (A/T/V); (L/T/V); (G/S); V; | |
| | (K/R); V; W; (P/R); (K/Q/R); A; I; P; Q; F; (M/N); | |
| | (I/V); G | |
| L479-2 | (G/P/S); (D/K/N/R/S/T); (A/S); (A/E/G/K/Q/V/T); | 354 |
| | (D/E); P; (F/L/R/S); (A/T/V/K); (L/T/V); (G/S); V; | |
| | (K/R); V; W; (P/R); (K/Q/R); A; I; P; Q; F; (M/N); | |
| | (I/V); G | |
| L479-3 | AIPQF[M/N][I/V]G | 355 |

SEQUENCES

Arabidopsis thaliana PPO1 (with transit peptide) (SEQ ID NO: 1)
MELSLLRPTTQSLLPSFSKPNLRLNVYKPLRLRCSVAGGPTVGSSKIEGGGGTTITTDCVIV
GGGISGLCIAQALATKHPDAAPNLIVTEAKDRVGGNIITREENGFLWEEGPNSFQPSDPMLT
MVVDSGLKDDLVLGDPTAPRFVLWNGKLRPVPSKLTDLPFFDLMSIGGKIRAGFGALGIRPS
PPGREESVEEFVRRNLGDEVFERLIEPFCSGVYAGDPSKLSMKAAFGKVWKLEQNGGSIIG
GTFKAIQERKNAPKAERDPRLPKPQGQTVGSFRKGLRMLPEAISARLGSKVKLSWKLSGIT
KLESGGYNLTYETPDGLVSVQSKSVVMTVPSHVASGLLRPLSESAANALSKLYYPPVAAVSI
SYPKEAIRTECLIDGELKGFGQLHPRTQGVETLGTIYSSSLFPNRAPPGRILLLNYIGGSTNT
GILSKSEGELVEAVDRDLRKMLIKPNSTDPLKLGVRVWPQAIPQFLVGHFDILDTAKSSLTSS
GYEGLFLGGNYVAGVALGRCVEGAYETAIEVNNFMSRYAYK*
Setaria italica PPO1 (with transit peptide) (SEQ ID NO: 2)
MVAAAMATAPSAGVPPLRGTRGPARFRIRGVSVRCAAVAGGAAEAPASAGARVSADCVVV
GGGISGLCTAQALATKHGVGDVLVTEARARPGGNITTVERPDEGYLWEEGPNSFQPSDPV
LTMAVDSGLKDDLVFGDPNAPRFVLWEGKLRPVPSKPADLPFFDLMSIPGKLRAGFGALGI
RPPPPGREESVEEFVRRNLGAEVFERLIEPFCSGVYAGDPSKLSMKAAFGKVWRLEEAGG
SIIGGTIKTIQERGKNPKPPRDPRLPTPKGQTVASFRKGLAMLPNAITSSLGSKVKLSWKLTSI
TKSDGMGYVLVYETPEGVVSVQAKSVIMTIPSYVASDILRPLSSDAADALSRFYYPPVAAVTI
SYPKEAIRKECLIDGELQGFGQLHPRSQGVETLGTIYSSSLFPNRAPAGRVLLLNYIGGATNT
GIVSKSASELVEAVDRDLRKMLINPSAVDPLVLGVRVWPQAIPQFLVGHLDLLEAAKSSLDR
GGYDGLFLGGNYVAGVALGRCVEGAYESASQISDFLTKYAYK*
pET24A AtPPO1 (SEQ ID NO: 3)
GCGGCCGCACTCGAGCACCACCACCACCACCACTGAGATCCGGCTGCTAACAAAGCC
CGAAAGGAAGCTGAGTTGGCTGCTGCCACCGCTGAGCAATAACTAGCATAACCCCTTG
GGGCCTCTAAACGGGTCTTGAGGGGTTTTTTGCTGAAAGGAGGAACTATATCCGGATT
GGCGAATGGGACGCGCCCTGTAGCGGCGCATTAAGCGCGGCGGGTGTGGTGGTTAC
GCGCAGCGTGACCGCTACACTTGCCAGCGCCCTAGCGCCCGCTCCTTTCGCTTTCTTC
CCTTCCTTTCTCGCCACGTTCGCCGGCTTTCCCCGTCAAGCTCTAAATCGGGGGCTCC
CTTTAGGGTTCCGATTTAGTGCTTTACGGCACCTCGACCCCAAAAAACTTGATTAGGGT
GATGGTTCACGTAGTGGGCCATCGCCCTGATAGACGGTTTTTCGCCCTTTGACGTTGG
AGTCCACGTTCTTTAATAGTGGACTCTTGTTCCAAACTGGAACAACACTCAACCCTATCT
CGGTCTATTCTTTTGATTTATAAGGGATTTTGCCGATTTCGGCCTATTGGTTAAAAAATG
AGCTGATTTAACAAAAATTTAACGCGAATTTTAACAAAATATTAACGTTTACAATTTCAGG
TGGCACTTTTCGGGGAAATGTGCGCGGAACCCCTATTTGTTTATTTTTCTAAATACATTC
AAATATGTATCCGCTCATGAATTAATTCTTAGAAAAACTCATCGAGCATCAAATGAAACT
GCAATTTATTCATATCAGGATTATCAATACCATATTTTTGAAAAAGCCGTTTCTGTAATGA
AGGAGAAAACTCACCGAGGCAGTTCCATAGGATGGCAAGATCCTGGTATCGGTCTGCG
ATTCCGACTCGTCCAACATCAATACAACCTATTAATTTCCCCTCGTCAAAAATAAGGTTA
TCAAGTGAGAAATCACCATGAGTGACGACTGAATCCGGTGAGAATGGCAAAAGTTTATG
CATTTCTTTCCAGACTTGTTCAACAGGCCAGCCATTACGCTCGTCATCAAAATCACTCG
CATCAACCAAACCGTTATTCATTCGTGATTGCGCCTGAGCGAGACGAAATACGCGATCG
CTGTTAAAAGGACAATTACAAACAGGAATCGAATGCAACCGGCGCAGGAACACTGCCA
GCGCATCAACAATATTTTCACCTGAATCAGGATATTCTTCTAATACCTGGAATGCTGTTT
TCCCGGGGATCGCAGTGGTGAGTAACCATGCATCATCAGGAGTACGGATAAAATGCTT
GATGGTCGGAAGAGGCATAAATTCCGTCAGCCAGTTTAGTCTGACCATCTCATCTGTAA
CATCATTGGCAACGCTACCTTTGCCATGTTTCAGAAACAACTCTGGCGCATCGGGCTTC
CCATACAATCGATAGATTGTCGCACCTGATTGCCCGACATTATCGCGAGCCCATTTATA
CCCATATAAATCAGCATCCATGTTGGAATTTAATCGCGGCCTAGAGCAAGACGTTTCCC
GTTGAATATGGCTCATAACACCCCTTGTATTACTGTTTATGTAAGCAGACAGTTTTATTG
TTCATGACCAAAATCCCTTAACGTGAGTTTTCGTTCCACTGAGCGTCAGACCCCGTAGA
AAAGATCAAAGGATCTTCTTGAGATCCTTTTTTTCTGCGCGTAATCTGCTGCTTGCAAAC
AAAAAAACCACCGCTACCAGCGGTGGTTTGTTTGCCGGATCAAGAGCTACCAACTCTTT
TTCCGAAGGTAACTGGCTTCAGCAGAGCGCAGATACCAAATACTGTCCTTCTAGTGTAG
CCGTAGTTAGGCCACCACTTCAAGAACTCTGTAGCACCGCCTACATACCTCGCTCTGCT
AATCCTGTTACCAGTGGCTGCTGCCAGTGGCGATAAGTCGTGTCTTACCGGGTTGGAC
TCAAGACGATAGTTACCGGATAAGGCGCAGCGGTCGGGCTGAACGGGGGGTTCGTGC
ACACAGCCCAGCTTGGAGCGAACGACCTACACCGAACTGAGATACCTACAGCGTGAGC
TATGAGAAAGCGCCACGCTTCCCGAAGGGAGAAAGGCGGACAGGTATCCGGTAAGCG
GCAGGGTCGGAACAGGAGAGCGCACGAGGGAGCTTCCAGGGGGAAACGCCTGGTAT
CTTTATAGTCCTGTCGGGTTTCGCCACCTCTGACTTGAGCGTCGATTTTTGTGATGCTC
GTCAGGGGGGCGGAGCCTATGGAAAAACGCCAGCAACGCGGCCTTTTTACGGTTCCT
GGCCTTTTGCTGGCCTTTTGCTCACATGTTCTTTCCTGCGTTATCCCCTGATTCTGTGGA
TAACCGTATTACCGCCTTTGAGTGAGCTGATACCGCTCGCCGCAGCCGAACGACCGAG
CGCAGCGAGTCAGTGAGCGAGGAAGCGGAAGAGCGCCTGATGCGGTATTTTCTCCTTA
CGCATCTGTGCGGTATTTCACACCGCATATATGGTGCACTCTCAGTACAATCTGCTCTG
ATGCCGCATAGTTAAGCCAGTATACACTCCGCTATCGCTACGTGACTGGGTCATGGCT
GCGCCCCGACACCCGCCAACACCCGCTGACGCGCCCTGACGGGCTTGTCTGCTCCCG
GCATCCGCTTACAGACAAGCTGTGACCGTCTCCGGGAGCTGCATGTGTCAGAGGTTTT
CACCGTCATCACCGAAACGCGCGAGGCAGCTGCGGTAAAGCTCATCAGCGTGGTCGT
GAAGCGATTCACAGATGTCTGCCTGTTCATCCGCGTCCAGCTCGTTGAGTTTCTCCAGA
AGCGTTAATGTCTGGCTTCTGATAAAGCGGGCCATGTTAAGGGCGGTTTTTTCCTGTTT
GGTCACTGATGCCTCCGTGTAAGGGGGATTTCTGTTCATGGGGGTAATGATACCGATG
AAACGAGAGAGGATGCTCACGATACGGGTTACTGATGATGAACATGCCCGGTTACTGG
AACGTTGTGAGGGTAAACAACTGGCGGTATGGATGCGGCGGGACCAGAGAAAAATCAC
TCAGGGTCAATGCCAGCGCTTCGTTAATACAGATGTAGGTGTTCCACAGGGTAGCCAG
CAGCATCCTGCGATGCAGATCCGGAACATAATGGTGCAGGGCGCTGACTTCCGCGTTT
CCAGACTTTACGAAACACGGAAACCGAAGACCATTCATGTTGTTGCTCAGGTCGCAGAC
GTTTTGCAGCAGCAGTCGCTTCACGTTCGCTCGCGTATCGGTGATTCATTCTGCTAACC
AGTAAGGCAACCCCGCCAGCCTAGCCGGGTCCTCAACGACAGGAGCACGATCATGCG
CACCCGTGGGGCCGCCATGCCGGCGATAATGGCCTGCTTCTCGCCGAAACGTTTGGT
GGCGGGACCAGTGACGAAGGCTTGAGCGAGGGCGTGCAAGATTCCGAATACCGCAAG
CGACAGGCCGATCATCGTCGCGCTCCAGCGAAAGCGGTCCTCGCCGAAAATGACCCA
GAGCGCTGCCGGCACCTGTCCTACGAGTTGCATGATAAAGAAGACAGTCATAAGTGCG
GCGACGATAGTCATGCCCCGCGCCCACCGGAAGGAGCTGACTGGGTTGAAGGCTCTC
AAGGGCATCGGTCGAGATCCCGGTGCCTAATGAGTGAGCTAACTTACATTAATTGCGTT
GCGCTCACTGCCCGCTTTCCAGTCGGGAAACCTGTCGTGCCAGCTGCATTAATGAATC
GGCCAACGCGCGGGGAGAGGCGGTTTGCGTATTGGGCGCCAGGGTGGTTTTTCTTTT
CACCAGTGAGACGGGCAACAGCTGATTGCCCTTCACCGCCTGGCCCTGAGAGAGTTG
CAGCAAGCGGTCCACGCTGGTTTGCCCCAGCAGGCGAAAATCCTGTTTGATGGTGGTT
AACGGCGGGATATAACATGAGCTGTCTTCGGTATCGTCGTATCCCACTACCGAGATATC
CGCACCAACGCGCAGCCCGGACTCGGTAATGGCGCGCATTGCGCCCAGCGCCATCTG
ATCGTTGGCAACCAGCATCGCAGTGGGAACGATGCCCTCATTCAGCATTTGCATGGTTT
GTTGAAAACCGGACATGGCACTCCAGTCGCCTTCCCGTTCCGCTATCGGCTGAATTTG
ATTGCGAGTGAGATATTTATGCCAGCCAGCCAGACGCAGACGCGCCGAGACAGAACTT
AATGGGCCCGCTAACAGCGCGATTTGCTGGTGACCCAATGCGACCAGATGCTCCACGC
CCAGTCGCGTACCGTCTTCATGGGAGAAAATAATACTGTTGATGGGTGTCTGGTCAGA
GACATCAAGAAATAACGCCGGAACATTAGTGCAGGCAGCTTCCACAGCAATGGCATCC
TGGTCATCCAGCGGATAGTTAATGATCAGCCCACTGACGCGTTGCGCGAGAAGATTGT
GCACCGCCGCTTTACAGGCTTCGACGCCGCTTCGTTCTACCATCGACACCACCACGCT
GGCACCCAGTTGATCGGCGCGAGATTTAATCGCCGCGACAATTTGCGACGGCGCGTG
CAGGGCCAGACTGGAGGTGGCAACGCCAATCAGCAACGACTGTTTGCCCGCCAGTTG
TTGTGCCACGCGGTTGGGAATGTAATTCAGCTCCGCCATCGCCGCTTCCACTTTTTCCC
GCGTTTTCGCAGAAACGTGGCTGGCCTGGTTCACCACGCGGGAAACGGTCTGATAAGA
GACACCGGCATACTCTGCGACATCGTATAACGTTACTGGTTTCACATTCACCACCCTGA
ATTGACTCTCTTCCGGGCGCTATCATGCCATACCGCGAAAGGTTTTGCGCCATTCGATG
GTGTCCGGGATCTCGACGCTCTCCCTTATGCGACTCCTGCATTAGGAAGCAGCCCAGT
AGTAGGTTGAGGCCGTTGAGCACCGCCGCCGCAAGGAATGGTGCATGCAAGGAGATG
GCGCCCAACAGTCCCCCGGCCACGGGGCCTGCCACCATACCCACGCCGAAACAAGCG
CTCATGAGCCCGAAGTGGCGAGCCCGATCTTCCCCATCGGTGATGTCGGCGATATAGG
CGCCAGCAACCGCACCTGTGGCGCCGGTGATGCCGGCCACGATGCGTCCGGCGTAGA
GGATCGAGATCTCGATCCCGCGAAATTAATACGACTCACTATAGGGGAATTGTGAGCG
GATAACAATTCCCCTCTAGAAATAATTTTGTTTAACTTTAAGAAGGAGATATACATATGGA
ACTTTCTTTGTTGCGCCCGACTACACAATCCCTGCTGCCCTCTTTTAGCAAGCCGAATC
TGCGTCTGAATGTGTATAAGCCACTGCGCCTGCGCTGTTCTGTGGCAGGCGGGCCGAC
GGTGGGGTCGTCGAAGATCGAGGGTGGCGGCGGCACTACGATTACCACCGACTGCGT
TATCGTGGGCGGGGGCATCAGCGGCTTGTGCATCGCACAAGCCCTGGCCACGAAACA
TCCCGATGCAGCACCCAATCTGATCGTAACGGAAGCGAAGGATCGCGTGGGTGGGAA
CATTATTACACGCGAAGAGAATGGTTTTCTGTGGGAAGAAGGGCCTAATTCCTTTCAGC
CAAGCGATCCGATGTTAACCATGGTAGTGGACTCAGGCCTGAAAGATGACCTCGTGCT
CGGCGACCCGACCGCACCGCGTTTCGTCCTGTGGAATGGCAAACTTCGCCCGGTGCC
GTCTAAACTGACCGACCTGCCGTTCTTTGACCTCATGTCCATCGGGGGTAAAATCCGTG
CTGGCTTTGGCGCTCTCGGGATTCGTCCGTCTCCGCCGGGTCGCGAAGAAAGTGTAGA
AGAGTTCGTCCGCCGTAATCTGGGCGATGAAGTCTTTGAGCGTCTGATCGAGCCATTC
TGTAGTGGTGTTTATGCTGGTGATCCGAGTAAACTGTCCATGAAGGCCGCGTTTGGCAA
GGTGTGGAAACTGGAGCAGAATGGAGGTAGCATCATCGGGGGTACATTCAAAGCAATT
CAAGAACGGAAAAATGCTCCCAAAGCAGAACGTGATCCGCGGCTCCCGAAACCGCAAG
GTCAGACCGTTGGTTCCTTCCGCAAAGGACTTCGCATGCTGCCGGAAGCCATCAGCGC
GCGTTTGGGAAGCAAAGTAAAATTGTCTTGGAAACTGTCGGGTATTACGAAACTGGAAT
CGGGGGGTTATAATTTGACATATGAAACCCCAGATGGTTTGGTGTCAGTGCAGAGCAA
GAGCGTTGTCATGACCGTGCCCTCACATGTCGCCTCCGGGTTGTTGCGTCCGCTGAGC
GAAAGCGCAGCGAACGCCCTCTCTAAACTTTACTATCCGCCTGTTGCCGCCGTGAGCA
TTAGTTATCCTAAGGAAGCTATTCGCACCGAATGTCTGATTGATGGTGAGCTCAAGGGT
TTTGGTCAGCTGCACCCGCGGACTCAGGGCGTCGAGACTTTAGGTACCATCTACTCCT
CTTCTCTGTTTCCGAACCGCGCACCGCCGGGCCGCATTCTGCTGCTGAATTATATTGGT
GGTAGTACGAATACCGGAATTTTATCGAAAAGTGAAGGTGAATTGGTCGAAGCCGTCGA
TCGTGACCTTCGCAAAATGCTGATTAAACCGAACTCGACCGACCCGCTGAAGTTAGGC
GTGCGTGTGTGGCCCCAAGCGATCCCACAGTTTCTGGTTGGTCACTTCGATATTCTGG
ATACCGCCAAATCCAGCCTGACAAGTAGCGGCTATGAAGGGCTGTTCCTGGGGGGAA
CTATGTCGCTGGTGTTGCGTTAGGGCGTTGTGTGGAAGGCGCGTATGAAACCGCCATT
GAAGTTAACAACTTCATGAGCCGCTACGCGTATAAATAA
(with transit peptide)
Arabidopsis thaliana PPO1 G64A (SEQ ID NO: 4)
MELSLLRPTTQSLLPSFSKPNLRLNVYKPLRLRCSVAGGPTVGSSKIEGGGGTTITTDCVIV
GAGISGLCIAQALATKHPDAAPNLIVTEAKDRVGGNIITREENGFLWEEGPNSFQPSDPMLT
MVVDSGLKDDLVLGDPTAPRFVLWNGKLRPVPSKLTDLPFFDLMSIGGKIRAGFGALGIRPS
PPGREESVEEFVRRNLGDEVFERLIEPFCSGVYAGDPSKLSMKAAFGKVWKLEQNGGSIIG
GTFKAIQERKNAPKAERDPRLPKPQGQTVGSFRKGLRMLPEAISARLGSKVKLSWKLSGIT
KLESGGYNLTYETPDGLVSVQSKSVVMTVPSHVASGLLRPLSESAANALSKLYYPPVAAVSI
SYPKEAIRTECLIDGELKGFGQLHPRTQGVETLGTIYSSSLFPNRAPPGRILLLNYIGGSTNT
GILSKSEGELVEAVDRDLRKMLIKPNSTDPLKLGVRVWPQAIPQFLVGHFDILDTAKSSLTSS
GYEGLFLGGNYVAGVALGRCVEGAYETAIEVNNFMSRYAYK*
Arabidopsis thaliana PPO1 V88I (SEQ ID NO: 5)
MELSLLRPTTQSLLPSFSKPNLRLNVYKPLRLRCSVAGGPTVGSSKIEGGGGTTITTDCVIV
GGGISGLCIAQALATKHPDAAPNLIITEAKDRVGGNIITREENGFLWEEGPNSFQPSDPMLT
MVVDSGLKDDLVLGDPTAPRFVLWNGKLRPVPSKLTDLPFFDLMSIGGKIRAGFGALGIRPS
PPGREESVEEFVRRNLGDEVFERLIEPFCSGVYAGDPSKLSMKAAFGKVWKLEQNGGSIIG
GTFKAIQERKNAPKAERDPRLPKPQGQTVGSFRKGLRMLPEAISARLGSKVKLSWKLSGIT
KLESGGYNLTYETPDGLVSVQSKSVVMTVPSHVASGLLRPLSESAANALSKLYYPPVAAVSI
SYPKEAIRTECLIDGELKGFGQLHPRTQGVETLGTIYSSSLFPNRAPPGRILLLNYIGGSTNT
GILSKSEGELVEAVDRDLRKMLIKPNSTDPLKLGVRVWPQAIPQFLVGHFDILDTAKSSLTSS
GYEGLFLGGNYVAGVALGRCVEGAYETAIEVNNFMSRYAYK*
Arabidopsis thaliana PPO1 E111D (SEQ ID NO: 6)
MELSLLRPTTQSLLPSFSKPNLRLNVYKPLRLRCSVAGGPTVGSSKIEGGGGTTITTDCVIV
GGGISGLCIAQALATKHPDAAPNLIVTEAKDRVGGNIITREENGFLWEDGPNSFQPSDPMLT
MVVDSGLKDDLVLGDPTAPRFVLWNGKLRPVPSKLTDLPFFDLMSIGGKIRAGFGALGIRPS
PPGREESVEEFVRRNLGDEVFERLIEPFCSGVYAGDPSKLSMKAAFGKVWKLEQNGGSIIG
GTFKAIQERKNAPKAERDPRLPKPQGQTVGSFRKGLRMLPEAISARLGSKVKLSWKLSGIT
KLESGGYNLTYETPDGLVSVQSKSVVMTVPSHVASGLLRPLSESAANALSKLYYPPVAAVSI
SYPKEAIRTECLIDGELKGFGQLHPRTQGVETLGTIYSSSLFPNRAPPGRILLLNYIGGSTNT
GILSKSEGELVEAVDRDLRKMLIKPNSTDPLKLGVRVWPQAIPQFLVGHFDILDTAKSSLTSS
GYEGLFLGGNYVAGVALGRCVEGAYETAIEVNNFMSRYAYK*
Arabidopsis thaliana PPO1 E111F (SEQ ID NO: 7)
MELSLLRPTTQSLLPSFSKPNLRLNVYKPLRLRCSVAGGPTVGSSKIEGGGGTTITTDCVIV
GGGISGLCIAQALATKHPDAAPNLIVTEAKDRVGGNIITREENGFLWEFGPNSFQPSDPMLT
MVVDSGLKDDLVLGDPTAPRFVLWNGKLRPVPSKLTDLPFFDLMSIGGKIRAGFGALGIRPS
PPGREESVEEFVRRNLGDEVFERLIEPFCSGVYAGDPSKLSMKAAFGKVWKLEQNGGSIIG
GTFKAIQERKNAPKAERDPRLPKPQGQTVGSFRKGLRMLPEAISARLGSKVKLSWKLSGIT
KLESGGYNLTYETPDGLVSVQSKSVVMTVPSHVASGLLRPLSESAANALSKLYYPPVAAVSI
SYPKEAIRTECLIDGELKGFGQLHPRTQGVETLGTIYSSSLFPNRAPPGRILLLNYIGGSTNT
GILSKSEGELVEAVDRDLRKMLIKPNSTDPLKLGVRVWPQAIPQFLVGHFDILDTAKSSLTSS
GYEGLFLGGNYVAGVALGRCVEGAYETAIEVNNFMSRYAYK*
Arabidopsis thaliana PPO1 V127I (SEQ ID NO: 8)
MELSLLRPTTQSLLPSFSKPNLRLNVYKPLRLRCSVAGGPTVGSSKIEGGGGTTITTDCVIV
GGGISGLCIAQALATKHPDAAPNLIVTEAKDRVGGNIITREENGFLWEEGPNSFQPSDPMLT
MVIDSGLKDDLVLGDPTAPRFVLWNGKLRPVPSKLTDLPFFDLMSIGGKIRAGFGALGIRPS
PPGREESVEEFVRRNLGDEVFERLIEPFCSGVYAGDPSKLSMKAAFGKVWKLEQNGGSIIG
GTFKAIQERKNAPKAERDPRLPKPQGQTVGSFRKGLRMLPEAISARLGSKVKLSWKLSGIT
KLESGGYNLTYETPDGLVSVQSKSVVMTVPSHVASGLLRPLSESAANALSKLYYPPVAAVSI
SYPKEAIRTECLIDGELKGFGQLHPRTQGVETLGTIYSSSLFPNRAPPGRILLLNYIGGSTNT
GILSKSEGELVEAVDRDLRKMLIKPNSTDPLKLGVRVWPQAIPQFLVGHFDILDTAKSSLTSS
GYEGLFLGGNYVAGVALGRCVEGAYETAIEVNNFMSRYAYK*
Arabidopsis thaliana PPO1 V127L (SEQ ID NO: 9)
MELSLLRPTTQSLLPSFSKPNLRLNVYKPLRLRCSVAGGPTVGSSKIEGGGGTTITTDCVIV
GGGISGLCIAQALATKHPDAAPNLIVTEAKDRVGGNIITREENGFLWEEGPNSFQPSDPMLT
MVLDSGLKDDLVLGDPTAPRFVLWNGKLRPVPSKLTDLPFFDLMSIGGKIRAGFGALGIRPS
PPGREESVEEFVRRNLGDEVFERLIEPFCSGVYAGDPSKLSMKAAFGKVWKLEQNGGSIIG
GTFKAIQERKNAPKAERDPRLPKPQGQTVGSFRKGLRMLPEAISARLGSKVKLSWKLSGIT
KLESGGYNLTYETPDGLVSVQSKSVVMTVPSHVASGLLRPLSESAANALSKLYYPPVAAVSI
SYPKEAIRTECLIDGELKGFGQLHPRTQGVETLGTIYSSSLFPNRAPPGRILLLNYIGGSTNT
GILSKSEGELVEAVDRDLRKMLIKPNSTDPLKLGVRVWPQAIPQFLVGHFDILDTAKSSLTSS
GYEGLFLGGNYVAGVALGRCVEGAYETAIEVNNFMSRYAYK*
Arabidopsis thaliana PPO1 P154Y (SEQ ID NO: 10)
MELSLLRPTTQSLLPSFSKPNLRLNVYKPLRLRCSVAGGPTVGSSKIEGGGGTTITTDCVIV
GGGISGLCIAQALATKHPDAAPNLIVTEAKDRVGGNIITREENGFLWEEGPNSFQPSDPMLT
MVVDSGLKDDLVLGDPTAPRFVLWNGKLRYVPSKLTDLPFFDLMSIGGKIRAGFGALGIRPS
PPGREESVEEFVRRNLGDEVFERLIEPFCSGVYAGDPSKLSMKAAFGKVWKLEQNGGSIIG
GTFKAIQERKNAPKAERDPRLPKPQGQTVGSFRKGLRMLPEAISARLGSKVKLSWKLSGIT
KLESGGYNLTYETPDGLVSVQSKSVVMTVPSHVASGLLRPLSESAANALSKLYYPPVAAVSI
SYPKEAIRTECLIDGELKGFGQLHPRTQGVETLGTIYSSSLFPNRAPPGRILLLNYIGGSTNT
GILSKSEGELVEAVDRDLRKMLIKPNSTDPLKLGVRVWPQAIPQFLVGHFDILDTAKSSLTSS
GYEGLFLGGNYVAGVALGRCVEGAYETAIEVNNFMSRYAYK*
Arabidopsis thaliana PPO1 K173H (SEQ ID NO: 11)
MELSLLRPTTQSLLPSFSKPNLRLNVYKPLRLRCSVAGGPTVGSSKIEGGGGTTITTDCVIV
GGGISGLCIAQALATKHPDAAPNLIVTEAKDRVGGNIITREENGFLWEEGPNSFQPSDPMLT
MVVDSGLKDDLVLGDPTAPRFVLWNGKLRPVPSKLTDLPFFDLMSIGGHIRAGFGALGIRPS
PPGREESVEEFVRRNLGDEVFERLIEPFCSGVYAGDPSKLSMKAAFGKVWKLEQNGGSIIG
GTFKAIQERKNAPKAERDPRLPKPQGQTVGSFRKGLRMLPEAISARLGSKVKLSWKLSGIT
KLESGGYNLTYETPDGLVSVQSKSVVMTVPSHVASGLLRPLSESAANALSKLYYPPVAAVSI
SYPKEAIRTECLIDGELKGFGQLHPRTQGVETLGTIYSSSLFPNRAPPGRILLLNYIGGSTNT
GILSKSEGELVEAVDRDLRKMLIKPNSTDPLKLGVRVWPQAIPQFLVGHFDILDTAKSSLTSS
GYEGLFLGGNYVAGVALGRCVEGAYETAIEVNNFMSRYAYK*
Arabidopsis thaliana PPO1 A176L (SEQ ID NO: 12)
MELSLLRPTTQSLLPSFSKPNLRLNVYKPLRLRCSVAGGPTVGSSKIEGGGGTTITTDCVIV
GGGISGLCIAQALATKHPDAAPNLIVTEAKDRVGGNIITREENGFLWEEGPNSFQPSDPMLT
MVVDSGLKDDLVLGDPTAPRFVLWNGKLRPVPSKLTDLPFFDLMSIGGKIRLGFGALGIRPS
PPGREESVEEFVRRNLGDEVFERLIEPFCSGVYAGDPSKLSMKAAFGKVWKLEQNGGSIIG
GTFKAIQERKNAPKAERDPRLPKPQGQTVGSFRKGLRMLPEAISARLGSKVKLSWKLSGIT
KLESGGYNLTYETPDGLVSVQSKSVVMTVPSHVASGLLRPLSESAANALSKLYYPPVAAVSI
SYPKEAIRTECLIDGELKGFGQLHPRTQGVETLGTIYSSSLFPNRAPPGRILLLNYIGGSTNT
GILSKSEGELVEAVDRDLRKMLIKPNSTDPLKLGVRVWPQAIPQFLVGHFDILDTAKSSLTSS
GYEGLFLGGNYVAGVALGRCVEGAYETAIEVNNFMSRYAYK*
Arabidopsis thaliana PPO1 V198W (SEQ ID NO: 13)
MELSLLRPTTQSLLPSFSKPNLRLNVYKPLRLRCSVAGGPTVGSSKIEGGGGTTITTDCVIV
GGGISGLCIAQALATKHPDAAPNLIVTEAKDRVGGNIITREENGFLWEEGPNSFQPSDPMLT
MVVDSGLKDDLVLGDPTAPRFVLWNGKLRPVPSKLTDLPFFDLMSIGGKIRAGFGALGIRPS
PPGREESVEEFWRRNLGDEVFERLIEPFCSGVYAGDPSKLSMKAAFGKVWKLEQNGGSII
GGTFKAIQERKNAPKAERDPRLPKPQGQTVGSFRKGLRMLPEAISARLGSKVKLSWKLSGI
TKLESGGYNLTYETPDGLVSVQSKSVVMTVPSHVASGLLRPLSESAANALSKLYYPPVAAV
SISYPKEAIRTECLIDGELKGFGQLHPRTQGVETLGTIYSSSLFPNRAPPGRILLLNYIGGSTN
TGILSKSEGELVEAVDRDLRKMLIKPNSTDPLKLGVRVWPQAIPQFLVGHFDILDTAKSSLTS
SGYEGLFLGGNYVAGVALGRCVEGAYETAIEVNNFMSRYAYK*
Arabidopsis thaliana PPO1 V206G (SEQ ID NO: 14)
MELSLLRPTTQSLLPSFSKPNLRLNVYKPLRLRCSVAGGPTVGSSKIEGGGGTTITTDCVIV
GGGISGLCIAQALATKHPDAAPNLIVTEAKDRVGGNIITREENGFLWEEGPNSFQPSDPMLT
MVVDSGLKDDLVLGDPTAPRFVLWNGKLRPVPSKLTDLPFFDLMSIGGKIRAGFGALGIRPS
PPGREESVEEFVRRNLGDEGFERLIEPFCSGVYAGDPSKLSMKAAFGKVWKLEQNGGSIIG
GTFKAIQERKNAPKAERDPRLPKPQGQTVGSFRKGLRMLPEAISARLGSKVKLSWKLSGIT
KLESGGYNLTYETPDGLVSVQSKSVVMTVPSHVASGLLRPLSESAANALSKLYYPPVAAVSI
SYPKEAIRTECLIDGELKGFGQLHPRTQGVETLGTIYSSSLFPNRAPPGRILLLNYIGGSTNT
GILSKSEGELVEAVDRDLRKMLIKPNSTDPLKLGVRVWPQAIPQFLVGHFDILDTAKSSLTSS
GYEGLFLGGNYVAGVALGRCVEGAYETAIEVNNFMSRYAYK*
Arabidopsis thaliana PPO1 F207Y (SEQ ID NO: 15)
MELSLLRPTTQSLLPSFSKPNLRLNVYKPLRLRCSVAGGPTVGSSKIEGGGGTTITTDCVIV
GGGISGLCIAQALATKHPDAAPNLIVTEAKDRVGGNIITREENGFLWEEGPNSFQPSDPMLT
MVVDSGLKDDLVLGDPTAPRFVLWNGKLRPVPSKLTDLPFFDLMSIGGKIRAGFGALGIRPS
PPGREESVEEFVRRNLGDEVYERLIEPFCSGVYAGDPSKLSMKAAFGKVWKLEQNGGSIIG
GTFKAIQERKNAPKAERDPRLPKPQGQTVGSFRKGLRMLPEAISARLGSKVKLSWKLSGIT
KLESGGYNLTYETPDGLVSVQSKSVVMTVPSHVASGLLRPLSESAANALSKLYYPPVAAVSI
SYPKEAIRTECLIDGELKGFGQLHPRTQGVETLGTIYSSSLFPNRAPPGRILLLNYIGGSTNT
GILSKSEGELVEAVDRDLRKMLIKPNSTDPLKLGVRVWPQAIPQFLVGHFDILDTAKSSLTSS
GYEGLFLGGNYVAGVALGRCVEGAYETAIEVNNFMSRYAYK*
Arabidopsis thaliana PPO1 E212Y (SEQ ID NO: 16)
MELSLLRPTTQSLLPSFSKPNLRLNVYKPLRLRCSVAGGPTVGSSKIEGGGGTTITTDCVIV
GGGISGLCIAQALATKHPDAAPNLIVTEAKDRVGGNIITREENGFLWEEGPNSFQPSDPMLT
MVVDSGLKDDLVLGDPTAPRFVLWNGKLRPVPSKLTDLPFFDLMSIGGKIRAGFGALGIRPS
PPGREESVEEFVRRNLGDEVFERLIYPFCSGVYAGDPSKLSMKAAFGKVWKLEQNGGSIIG
GTFKAIQERKNAPKAERDPRLPKPQGQTVGSFRKGLRMLPEAISARLGSKVKLSWKLSGIT
KLESGGYNLTYETPDGLVSVQSKSVVMTVPSHVASGLLRPLSESAANALSKLYYPPVAAVSI
SYPKEAIRTECLIDGELKGFGQLHPRTQGVETLGTIYSSSLFPNRAPPGRILLLNYIGGSTNT
GILSKSEGELVEAVDRDLRKMLIKPNSTDPLKLGVRVWPQAIPQFLVGHFDILDTAKSSLTSS
GYEGLFLGGNYVAGVALGRCVEGAYETAIEVNNFMSRYAYK*
Arabidopsis thaliana PPO1 F214Y (SEQ ID NO: 17)
MELSLLRPTTQSLLPSFSKPNLRLNVYKPLRLRCSVAGGPTVGSSKIEGGGGTTITTDCVIV
GGGISGLCIAQALATKHPDAAPNLIVTEAKDRVGGNIITREENGFLWEEGPNSFQPSDPMLT
MVVDSGLKDDLVLGDPTAPRFVLWNGKLRPVPSKLTDLPFFDLMSIGGKIRAGFGALGIRPS
PPGREESVEEFVRRNLGDEVFERLIEPYCSGVYAGDPSKLSMKAAFGKVWKLEQNGGSIIG
GTFKAIQERKNAPKAERDPRLPKPQGQTVGSFRKGLRMLPEAISARLGSKVKLSWKLSGIT
KLESGGYNLTYETPDGLVSVQSKSVVMTVPSHVASGLLRPLSESAANALSKLYYPPVAAVSI
SYPKEAIRTECLIDGELKGFGQLHPRTQGVETLGTIYSSSLFPNRAPPGRILLLNYIGGSTNT
GILSKSEGELVEAVDRDLRKMLIKPNSTDPLKLGVRVWPQAIPQFLVGHFDILDTAKSSLTSS
GYEGLFLGGNYVAGVALGRCVEGAYETAIEVNNFMSRYAYK*
Arabidopsis thaliana PPO1 V218K (SEQ ID NO: 18)
MELSLLRPTTQSLLPSFSKPNLRLNVYKPLRLRCSVAGGPTVGSSKIEGGGGTTITTDCVIV
GGGISGLCIAQALATKHPDAAPNLIVTEAKDRVGGNIITREENGFLWEEGPNSFQPSDPMLT
MVVDSGLKDDLVLGDPTAPRFVLWNGKLRPVPSKLTDLPFFDLMSIGGKIRAGFGALGIRPS
PPGREESVEEFVRRNLGDEVFERLIEPFCSGKYAGDPSKLSMKAAFGKVWKLEQNGGSIIG
GTFKAIQERKNAPKAERDPRLPKPQGQTVGSFRKGLRMLPEAISARLGSKVKLSWKLSGIT
KLESGGYNLTYETPDGLVSVQSKSVVMTVPSHVASGLLRPLSESAANALSKLYYPPVAAVSI
SYPKEAIRTECLIDGELKGFGQLHPRTQGVETLGTIYSSSLFPNRAPPGRILLLNYIGGSTNT
GILSKSEGELVEAVDRDLRKMLIKPNSTDPLKLGVRVWPQAIPQFLVGHFDILDTAKSSLTSS
GYEGLFLGGNYVAGVALGRCVEGAYETAIEVNNFMSRYAYK*
Arabidopsis thaliana PPO1 Y219S (SEQ ID NO: 19)
MELSLLRPTTQSLLPSFSKPNLRLNVYKPLRLRCSVAGGPTVGSSKIEGGGGTTITTDCVIV
GGGISGLCIAQALATKHPDAAPNLIVTEAKDRVGGNIITREENGFLWEEGPNSFQPSDPMLT
MVVDSGLKDDLVLGDPTAPRFVLWNGKLRPVPSKLTDLPFFDLMSIGGKIRAGFGALGIRPS
PPGREESVEEFVRRNLGDEVFERLIEPFCSGVSAGDPSKLSMKAAFGKVWKLEQNGGSIIG
GTFKAIQERKNAPKAERDPRLPKPQGQTVGSFRKGLRMLPEAISARLGSKVKLSWKLSGIT
KLESGGYNLTYETPDGLVSVQSKSVVMTVPSHVASGLLRPLSESAANALSKLYYPPVAAVSI
SYPKEAIRTECLIDGELKGFGQLHPRTQGVETLGTIYSSSLFPNRAPPGRILLLNYIGGSTNT
GILSKSEGELVEAVDRDLRKMLIKPNSTDPLKLGVRVWPQAIPQFLVGHFDILDTAKSSLTSS
GYEGLFLGGNYVAGVALGRCVEGAYETAIEVNNFMSRYAYK*
Arabidopsis thaliana PPO1 A220C (SEQ ID NO: 20)
MELSLLRPTTQSLLPSFSKPNLRLNVYKPLRLRCSVAGGPTVGSSKIEGGGGTTITTDCVIV
GGGISGLCIAQALATKHPDAAPNLIVTEAKDRVGGNIITREENGFLWEEGPNSFQPSDPMLT
MVVDSGLKDDLVLGDPTAPRFVLWNGKLRPVPSKLTDLPFFDLMSIGGKIRAGFGALGIRPS
PPGREESVEEFVRRNLGDEVFERLIEPFCSGVYCGDPSKLSMKAAFGKVWKLEQNGGSIIG
GTFKAIQERKNAPKAERDPRLPKPQGQTVGSFRKGLRMLPEAISARLGSKVKLSWKLSGIT
KLESGGYNLTYETPDGLVSVQSKSVVMTVPSHVASGLLRPLSESAANALSKLYYPPVAAVSI
SYPKEAIRTECLIDGELKGFGQLHPRTQGVETLGTIYSSSLFPNRAPPGRILLLNYIGGSTNT
GILSKSEGELVEAVDRDLRKMLIKPNSTDPLKLGVRVWPQAIPQFLVGHFDILDTAKSSLTSS
GYEGLFLGGNYVAGVALGRCVEGAYETAIEVNNFMSRYAYK*
Arabidopsis thaliana PPO1 P271C (SEQ ID NO: 21)
MELSLLRPTTQSLLPSFSKPNLRLNVYKPLRLRCSVAGGPTVGSSKIEGGGGTTITTDCVIV
GGGISGLCIAQALATKHPDAAPNLIVTEAKDRVGGNIITREENGFLWEEGPNSFQPSDPMLT
MVVDSGLKDDLVLGDPTAPRFVLWNGKLRPVPSKLTDLPFFDLMSIGGKIRAGFGALGIRPS
PPGREESVEEFVRRNLGDEVFERLIEPFCSGVYAGDPSKLSMKAAFGKVWKLEQNGGSIIG
GTFKAIQERKNAPKAERDPRLPKCQGQTVGSFRKGLRMLPEAISARLGSKVKLSWKLSGIT
KLESGGYNLTYETPDGLVSVQSKSVVMTVPSHVASGLLRPLSESAANALSKLYYPPVAAVSI
SYPKEAIRTECLIDGELKGFGQLHPRTQGVETLGTIYSSSLFPNRAPPGRILLLNYIGGSTNT
GILSKSEGELVEAVDRDLRKMLIKPNSTDPLKLGVRVWPQAIPQFLVGHFDILDTAKSSLTSS
GYEGLFLGGNYVAGVALGRCVEGAYETAIEVNNFMSRYAYK*
Arabidopsis thaliana PPO1 Q272H (SEQ ID NO: 22)
MELSLLRPTTQSLLPSFSKPNLRLNVYKPLRLRCSVAGGPTVGSSKIEGGGGTTITTDCVIV
GGGISGLCIAQALATKHPDAAPNLIVTEAKDRVGGNIITREENGFLWEEGPNSFQPSDPMLT
MVVDSGLKDDLVLGDPTAPRFVLWNGKLRPVPSKLTDLPFFDLMSIGGKIRAGFGALGIRPS
PPGREESVEEFVRRNLGDEVFERLIEPFCSGVYAGDPSKLSMKAAFGKVWKLEQNGGSIIG
GTFKAIQERKNAPKAERDPRLPKPHGQTVGSFRKGLRMLPEAISARLGSKVKLSWKLSGIT
KLESGGYNLTYETPDGLVSVQSKSVVMTVPSHVASGLLRPLSESAANALSKLYYPPVAAVSI
SYPKEAIRTECLIDGELKGFGQLHPRTQGVETLGTIYSSSLFPNRAPPGRILLLNYIGGSTNT
GILSKSEGELVEAVDRDLRKMLIKPNSTDPLKLGVRVWPQAIPQFLVGHFDILDTAKSSLTSS
GYEGLFLGGNYVAGVALGRCVEGAYETAIEVNNFMSRYAYK*
Arabidopsis thaliana PPO1 Q272I (SEQ ID NO: 23)
MELSLLRPTTQSLLPSFSKPNLRLNVYKPLRLRCSVAGGPTVGSSKIEGGGGTTITTDCVIV
GGGISGLCIAQALATKHPDAAPNLIVTEAKDRVGGNIITREENGFLWEEGPNSFQPSDPMLT
MVVDSGLKDDLVLGDPTAPRFVLWNGKLRPVPSKLTDLPFFDLMSIGGKIRAGFGALGIRPS
PPGREESVEEFVRRNLGDEVFERLIEPFCSGVYAGDPSKLSMKAAFGKVWKLEQNGGSIIG
GTFKAIQERKNAPKAERDPRLPKPIGQTVGSFRKGLRMLPEAISARLGSKVKLSWKLSGITK
LESGGYNLTYETPDGLVSVQSKSVVMTVPSHVASGLLRPLSESAANALSKLYYPPVAAVSIS
YPKEAIRTECLIDGELKGFGQLHPRTQGVETLGTIYSSSLFPNRAPPGRILLLNYIGGSTNTGI
LSKSEGELVEAVDRDLRKMLIKPNSTDPLKLGVRVWPQAIPQFLVGHFDILDTAKSSLTSSG
YEGLFLGGNYVAGVALGRCVEGAYETAIEVNNFMSRYAYK*
Arabidopsis thaliana PPO1 Q272T (SEQ ID NO: 24)
MELSLLRPTTQSLLPSFSKPNLRLNVYKPLRLRCSVAGGPTVGSSKIEGGGGTTITTDCVIV
GGGISGLCIAQALATKHPDAAPNLIVTEAKDRVGGNIITREENGFLWEEGPNSFQPSDPMLT
MVVDSGLKDDLVLGDPTAPRFVLWNGKLRPVPSKLTDLPFFDLMSIGGKIRAGFGALGIRPS
PPGREESVEEFVRRNLGDEVFERLIEPFCSGVYAGDPSKLSMKAAFGKVWKLEQNGGSIIG
GTFKAIQERKNAPKAERDPRLPKPTGQTVGSFRKGLRMLPEAISARLGSKVKLSWKLSGITK
LESGGYNLTYETPDGLVSVQSKSVVMTVPSHVASGLLRPLSESAANALSKLYYPPVAAVSIS
YPKEAIRTECLIDGELKGFGQLHPRTQGVETLGTIYSSSLFPNRAPPGRILLLNYIGGSTNTGI
LSKSEGELVEAVDRDLRKMLIKPNSTDPLKLGVRVWPQAIPQFLVGHFDILDTAKSSLTSSG
YEGLFLGGNYVAGVALGRCVEGAYETAIEVNNFMSRYAYK*
Arabidopsis thaliana PPO1 K281C (SEQ ID NO: 25)
MELSLLRPTTQSLLPSFSKPNLRLNVYKPLRLRCSVAGGPTVGSSKIEGGGGTTITTDCVIV
GGGISGLCIAQALATKHPDAAPNLIVTEAKDRVGGNIITREENGFLWEEGPNSFQPSDPMLT
MVVDSGLKDDLVLGDPTAPRFVLWNGKLRPVPSKLTDLPFFDLMSIGGKIRAGFGALGIRPS
PPGREESVEEFVRRNLGDEVFERLIEPFCSGVYAGDPSKLSMKAAFGKVWKLEQNGGSIIG
GTFKAIQERKNAPKAERDPRLPKPQGQTVGSFRCGLRMLPEAISARLGSKVKLSWKLSGIT
KLESGGYNLTYETPDGLVSVQSKSVVMTVPSHVASGLLRPLSESAANALSKLYYPPVAAVSI
SYPKEAIRTECLIDGELKGFGQLHPRTQGVETLGTIYSSSLFPNRAPPGRILLLNYIGGSTNT
GILSKSEGELVEAVDRDLRKMLIKPNSTDPLKLGVRVWPQAIPQFLVGHFDILDTAKSSLTSS
GYEGLFLGGNYVAGVALGRCVEGAYETAIEVNNFMSRYAYK*
Arabidopsis thaliana PPO1 K281E (SEQ ID NO: 26)
MELSLLRPTTQSLLPSFSKPNLRLNVYKPLRLRCSVAGGPTVGSSKIEGGGGTTITTDCVIV
GGGISGLCIAQALATKHPDAAPNLIVTEAKDRVGGNIITREENGFLWEEGPNSFQPSDPMLT
MVVDSGLKDDLVLGDPTAPRFVLWNGKLRPVPSKLTDLPFFDLMSIGGKIRAGFGALGIRPS
PPGREESVEEFVRRNLGDEVFERLIEPFCSGVYAGDPSKLSMKAAFGKVWKLEQNGGSIIG
GTFKAIQERKNAPKAERDPRLPKPQGQTVGSFREGLRMLPEAISARLGSKVKLSWKLSGIT
KLESGGYNLTYETPDGLVSVQSKSVVMTVPSHVASGLLRPLSESAANALSKLYYPPVAAVSI
SYPKEAIRTECLIDGELKGFGQLHPRTQGVETLGTIYSSSLFPNRAPPGRILLLNYIGGSTNT
GILSKSEGELVEAVDRDLRKMLIKPNSTDPLKLGVRVWPQAIPQFLVGHFDILDTAKSSLTSS
GYEGLFLGGNYVAGVALGRCVEGAYETAIEVNNFMSRYAYK*
Arabidopsis thaliana PPO1 K281N (SEQ ID NO: 27)
MELSLLRPTTQSLLPSFSKPNLRLNVYKPLRLRCSVAGGPTVGSSKIEGGGGTTITTDCVIV
GGGISGLCIAQALATKHPDAAPNLIVTEAKDRVGGNIITREENGFLWEEGPNSFQPSDPMLT
MVVDSGLKDDLVLGDPTAPRFVLWNGKLRPVPSKLTDLPFFDLMSIGGKIRAGFGALGIRPS
PPGREESVEEFVRRNLGDEVFERLIEPFCSGVYAGDPSKLSMKAAFGKVWKLEQNGGSIIG
GTFKAIQERKNAPKAERDPRLPKPQGQTVGSFRNGLRMLPEAISARLGSKVKLSWKLSGIT
KLESGGYNLTYETPDGLVSVQSKSVVMTVPSHVASGLLRPLSESAANALSKLYYPPVAAVSI
SYPKEAIRTECLIDGELKGFGQLHPRTQGVETLGTIYSSSLFPNRAPPGRILLLNYIGGSTNT
GILSKSEGELVEAVDRDLRKMLIKPNSTDPLKLGVRVWPQAIPQFLVGHFDILDTAKSSLTSS
GYEGLFLGGNYVAGVALGRCVEGAYETAIEVNNFMSRYAYK*
Arabidopsis thaliana PPO1 R284G (SEQ ID NO: 28)
MELSLLRPTTQSLLPSFSKPNLRLNVYKPLRLRCSVAGGPTVGSSKIEGGGGTTITTDCVIV
GGGISGLCIAQALATKHPDAAPNLIVTEAKDRVGGNIITREENGFLWEEGPNSFQPSDPMLT
MVVDSGLKDDLVLGDPTAPRFVLWNGKLRPVPSKLTDLPFFDLMSIGGKIRAGFGALGIRPS
PPGREESVEEFVRRNLGDEVFERLIEPFCSGVYAGDPSKLSMKAAFGKVWKLEQNGGSIIG
GTFKAIQERKNAPKAERDPRLPKPQGQTVGSFRKGLGMLPEAISARLGSKVKLSWKLSGIT
KLESGGYNLTYETPDGLVSVQSKSVVMTVPSHVASGLLRPLSESAANALSKLYYPPVAAVSI
SYPKEAIRTECLIDGELKGFGQLHPRTQGVETLGTIYSSSLFPNRAPPGRILLLNYIGGSTNT
GILSKSEGELVEAVDRDLRKMLIKPNSTDPLKLGVRVWPQAIPQFLVGHFDILDTAKSSLTSS
GYEGLFLGGNYVAGVALGRCVEGAYETAIEVNNFMSRYAYK*
Arabidopsis thaliana PPO1 S305L (SEQ ID NO: 29)
MELSLLRPTTQSLLPSFSKPNLRLNVYKPLRLRCSVAGGPTVGSSKIEGGGGTTITTDCVIV
GGGISGLCIAQALATKHPDAAPNLIVTEAKDRVGGNIITREENGFLWEEGPNSFQPSDPMLT
MVVDSGLKDDLVLGDPTAPRFVLWNGKLRPVPSKLTDLPFFDLMSIGGKIRAGFGALGIRPS
PPGREESVEEFVRRNLGDEVFERLIEPFCSGVYAGDPSKLSMKAAFGKVWKLEQNGGSIIG
GTFKAIQERKNAPKAERDPRLPKPQGQTVGSFRKGLRMLPEAISARLGSKVKLSWKLLGITK
LESGGYNLTYETPDGLVSVQSKSVVMTVPSHVASGLLRPLSESAANALSKLYYPPVAAVSIS
YPKEAIRTECLIDGELKGFGQLHPRTQGVETLGTIYSSSLFPNRAPPGRILLLNYIGGSTNTGI
LSKSEGELVEAVDRDLRKMLIKPNSTDPLKLGVRVWPQAIPQFLVGHFDILDTAKSSLTSSG
YEGLFLGGNYVAGVALGRCVEGAYETAIEVNNFMSRYAYK*
Arabidopsis thaliana PPO1 L360C (SEQ ID NO: 30)
MELSLLRPTTQSLLPSFSKPNLRLNVYKPLRLRCSVAGGPTVGSSKIEGGGGTTITTDCVIV
GGGISGLCIAQALATKHPDAAPNLIVTEAKDRVGGNIITREENGFLWEEGPNSFQPSDPMLT
MVVDSGLKDDLVLGDPTAPRFVLWNGKLRPVPSKLTDLPFFDLMSIGGKIRAGFGALGIRPS
PPGREESVEEFVRRNLGDEVFERLIEPFCSGVYAGDPSKLSMKAAFGKVWKLEQNGGSIIG
GTFKAIQERKNAPKAERDPRLPKPQGQTVGSFRKGLRMLPEAISARLGSKVKLSWKLSGIT
KLESGGYNLTYETPDGLVSVQSKSVVMTVPSHVASGLLRPLSESAANALSKCYYPPVAAVS
ISYPKEAIRTECLIDGELKGFGQLHPRTQGVETLGTIYSSSLFPNRAPPGRILLLNYIGGSTNT
GILSKSEGELVEAVDRDLRKMLIKPNSTDPLKLGVRVWPQAIPQFLVGHFDILDTAKSSLTSS
GYEGLFLGGNYVAGVALGRCVEGAYETAIEVNNFMSRYAYK*
Arabidopsis thaliana PPO1 Y361D (SEQ ID NO: 31)
MELSLLRPTTQSLLPSFSKPNLRLNVYKPLRLRCSVAGGPTVGSSKIEGGGGTTITTDCVIV
GGGISGLCIAQALATKHPDAAPNLIVTEAKDRVGGNIITREENGFLWEEGPNSFQPSDPMLT
MVVDSGLKDDLVLGDPTAPRFVLWNGKLRPVPSKLTDLPFFDLMSIGGKIRAGFGALGIRPS
PPGREESVEEFVRRNLGDEVFERLIEPFCSGVYAGDPSKLSMKAAFGKVWKLEQNGGSIIG
GTFKAIQERKNAPKAERDPRLPKPQGQTVGSFRKGLRMLPEAISARLGSKVKLSWKLSGIT
KLESGGYNLTYETPDGLVSVQSKSVVMTVPSHVASGLLRPLSESAANALSKLDYPPVAAVSI
SYPKEAIRTECLIDGELKGFGQLHPRTQGVETLGTIYSSSLFPNRAPPGRILLLNYIGGSTNT
GILSKSEGELVEAVDRDLRKMLIKPNSTDPLKLGVRVWPQAIPQFLVGHFDILDTAKSSLTSS
GYEGLFLGGNYVAGVALGRCVEGAYETAIEVNNFMSRYAYK*
Arabidopsis thaliana PPO1 Y361F (SEQ ID NO: 32)
MELSLLRPTTQSLLPSFSKPNLRLNVYKPLRLRCSVAGGPTVGSSKIEGGGGTTITTDCVIV
GGGISGLCIAQALATKHPDAAPNLIVTEAKDRVGGNIITREENGFLWEEGPNSFQPSDPMLT
MVVDSGLKDDLVLGDPTAPRFVLWNGKLRPVPSKLTDLPFFDLMSIGGKIRAGFGALGIRPS
PPGREESVEEFVRRNLGDEVFERLIEPFCSGVYAGDPSKLSMKAAFGKVWKLEQNGGSIIG
GTFKAIQERKNAPKAERDPRLPKPQGQTVGSFRKGLRMLPEAISARLGSKVKLSWKLSGIT
KLESGGYNLTYETPDGLVSVQSKSVVMTVPSHVASGLLRPLSESAANALSKLFYPPVAAVSI
SYPKEAIRTECLIDGELKGFGQLHPRTQGVETLGTIYSSSLFPNRAPPGRILLLNYIGGSTNT
GILSKSEGELVEAVDRDLRKMLIKPNSTDPLKLGVRVWPQAIPQFLVGHFDILDTAKSSLTSS
GYEGLFLGGNYVAGVALGRCVEGAYETAIEVNNFMSRYAYK*
Arabidopsis thaliana PPO1 Y361P (SEQ ID NO: 33)
MELSLLRPTTQSLLPSFSKPNLRLNVYKPLRLRCSVAGGPTVGSSKIEGGGGTTITTDCVIV
GGGISGLCIAQALATKHPDAAPNLIVTEAKDRVGGNIITREENGFLWEEGPNSFQPSDPMLT
MVVDSGLKDDLVLGDPTAPRFVLWNGKLRPVPSKLTDLPFFDLMSIGGKIRAGFGALGIRPS
PPGREESVEEFVRRNLGDEVFERLIEPFCSGVYAGDPSKLSMKAAFGKVWKLEQNGGSIIG
GTFKAIQERKNAPKAERDPRLPKPQGQTVGSFRKGLRMLPEAISARLGSKVKLSWKLSGIT
KLESGGYNLTYETPDGLVSVQSKSVVMTVPSHVASGLLRPLSESAANALSKLPYPPVAAVSI
SYPKEAIRTECLIDGELKGFGQLHPRTQGVETLGTIYSSSLFPNRAPPGRILLLNYIGGSTNT
GILSKSEGELVEAVDRDLRKMLIKPNSTDPLKLGVRVWPQAIPQFLVGHFDILDTAKSSLTSS
GYEGLFLGGNYVAGVALGRCVEGAYETAIEVNNFMSRYAYK*
Arabidopsis thaliana PPO1 Y361Q (SEQ ID NO: 34)
MELSLLRPTTQSLLPSFSKPNLRLNVYKPLRLRCSVAGGPTVGSSKIEGGGGTTITTDCVIV
GGGISGLCIAQALATKHPDAAPNLIVTEAKDRVGGNIITREENGFLWEEGPNSFQPSDPMLT
MVVDSGLKDDLVLGDPTAPRFVLWNGKLRPVPSKLTDLPFFDLMSIGGKIRAGFGALGIRPS
PPGREESVEEFVRRNLGDEVFERLIEPFCSGVYAGDPSKLSMKAAFGKVWKLEQNGGSIIG
GTFKAIQERKNAPKAERDPRLPKPQGQTVGSFRKGLRMLPEAISARLGSKVKLSWKLSGIT
KLESGGYNLTYETPDGLVSVQSKSVVMTVPSHVASGLLRPLSESAANALSKLQYPPVAAVSI
SYPKEAIRTECLIDGELKGFGQLHPRTQGVETLGTIYSSSLFPNRAPPGRILLLNYIGGSTNT
GILSKSEGELVEAVDRDLRKMLIKPNSTDPLKLGVRVWPQAIPQFLVGHFDILDTAKSSLTSS
GYEGLFLGGNYVAGVALGRCVEGAYETAIEVNNFMSRYAYK*
Arabidopsis thaliana PPO1 Y361R (SEQ ID NO: 35)
MELSLLRPTTQSLLPSFSKPNLRLNVYKPLRLRCSVAGGPTVGSSKIEGGGGTTITTDCVIV
GGGISGLCIAQALATKHPDAAPNLIVTEAKDRVGGNIITREENGFLWEEGPNSFQPSDPMLT
MVVDSGLKDDLVLGDPTAPRFVLWNGKLRPVPSKLTDLPFFDLMSIGGKIRAGFGALGIRPS
PPGREESVEEFVRRNLGDEVFERLIEPFCSGVYAGDPSKLSMKAAFGKVWKLEQNGGSIIG
GTFKAIQERKNAPKAERDPRLPKPQGQTVGSFRKGLRMLPEAISARLGSKVKLSWKLSGIT
KLESGGYNLTYETPDGLVSVQSKSVVMTVPSHVASGLLRPLSESAANALSKLYYRPVAAVSI
SYPKEAIRTECLIDGELKGFGQLHPRTQGVETLGTIYSSSLFPNRAPPGRILLLNYIGGSTNT
GILSKSEGELVEAVDRDLRKMLIKPNSTDPLKLGVRVWPQAIPQFLVGHFDILDTAKSSLTSS
GYEGLFLGGNYVAGVALGRCVEGAYETAIEVNNFMSRYAYK*
Arabidopsis thaliana PPO1 Y361T (SEQ ID NO: 36)
MELSLLRPTTQSLLPSFSKPNLRLNVYKPLRLRCSVAGGPTVGSSKIEGGGGTTITTDCVIV
GGGISGLCIAQALATKHPDAAPNLIVTEAKDRVGGNIITREENGFLWEEGPNSFQPSDPMLT
MVVDSGLKDDLVLGDPTAPRFVLWNGKLRPVPSKLTDLPFFDLMSIGGKIRAGFGALGIRPS
PPGREESVEEFVRRNLGDEVFERLIEPFCSGVYAGDPSKLSMKAAFGKVWKLEQNGGSIIG
GTFKAIQERKNAPKAERDPRLPKPQGQTVGSFRKGLRMLPEAISARLGSKVKLSWKLSGIT
KLESGGYNLTYETPDGLVSVQSKSVVMTVPSHVASGLLRPLSESAANALSKLTYPPVAAVSI
SYPKEAIRTECLIDGELKGFGQLHPRTQGVETLGTIYSSSLFPNRAPPGRILLLNYIGGSTNT
GILSKSEGELVEAVDRDLRKMLIKPNSTDPLKLGVRVWPQAIPQFLVGHFDILDTAKSSLTSS
GYEGLFLGGNYVAGVALGRCVEGAYETAIEVNNFMSRYAYK*
Arabidopsis thaliana PPO1 Y362F (SEQ ID NO: 37)
MELSLLRPTTQSLLPSFSKPNLRLNVYKPLRLRCSVAGGPTVGSSKIEGGGGTTITTDCVIV
GGGISGLCIAQALATKHPDAAPNLIVTEAKDRVGGNIITREENGFLWEEGPNSFQPSDPMLT
MVVDSGLKDDLVLGDPTAPRFVLWNGKLRPVPSKLTDLPFFDLMSIGGKIRAGFGALGIRPS
PPGREESVEEFVRRNLGDEVFERLIEPFCSGVYAGDPSKLSMKAAFGKVWKLEQNGGSIIG
GTFKAIQERKNAPKAERDPRLPKPQGQTVGSFRKGLRMLPEAISARLGSKVKLSWKLSGIT
KLESGGYNLTYETPDGLVSVQSKSVVMTVPSHVASGLLRPLSESAANALSKLYFPPVAAVSI
SYPKEAIRTECLIDGELKGFGQLHPRTQGVETLGTIYSSSLFPNRAPPGRILLLNYIGGSTNT
GILSKSEGELVEAVDRDLRKMLIKPNSTDPLKLGVRVWPQAIPQFLVGHFDILDTAKSSLTSS
GYEGLFLGGNYVAGVALGRCVEGAYETAIEVNNFMSRYAYK*
Arabidopsis thaliana PPO1 Y365L (SEQ ID NO: 38)
MELSLLRPTTQSLLPSFSKPNLRLNVYKPLRLRCSVAGGPTVGSSKIEGGGGTTITTDCVIV
GGGISGLCIAQALATKHPDAAPNLIVTEAKDRVGGNIITREENGFLWEEGPNSFQPSDPMLT
MVVDSGLKDDLVLGDPTAPRFVLWNGKLRPVPSKLTDLPFFDLMSIGGKIRAGFGALGIRPS
PPGREESVEEFVRRNLGDEVFERLIEPFCSGVYAGDPSKLSMKAAFGKVWKLEQNGGSIIG
GTFKAIQERKNAPKAERDPRLPKPQGQTVGSFRKGLRMLPEAISARLGSKVKLSWKLSGIT
KLESGGYNLTYETPDGLVSVQSKSVVMTVPSHVASGLLRPLSESAANALSKLYYPPLAAVSI
SYPKEAIRTECLIDGELKGFGQLHPRTQGVETLGTIYSSSLFPNRAPPGRILLLNYIGGSTNT
GILSKSEGELVEAVDRDLRKMLIKPNSTDPLKLGVRVWPQAIPQFLVGHFDILDTAKSSLTSS
GYEGLFLGGNYVAGVALGRCVEGAYETAIEVNNFMSRYAYK*
Arabidopsis thaliana PPO1 Y365M (SEQ ID NO: 39)
MELSLLRPTTQSLLPSFSKPNLRLNVYKPLRLRCSVAGGPTVGSSKIEGGGGTTITTDCVIV
GGGISGLCIAQALATKHPDAAPNLIVTEAKDRVGGNIITREENGFLWEEGPNSFQPSDPMLT
MVVDSGLKDDLVLGDPTAPRFVLWNGKLRPVPSKLTDLPFFDLMSIGGKIRAGFGALGIRPS
PPGREESVEEFVRRNLGDEVFERLIEPFCSGVYAGDPSKLSMKAAFGKVWKLEQNGGSIIG
GTFKAIQERKNAPKAERDPRLPKPQGQTVGSFRKGLRMLPEAISARLGSKVKLSWKLSGIT
KLESGGYNLTYETPDGLVSVQSKSVVMTVPSHVASGLLRPLSESAANALSKLYYPPMAAVS
ISYPKEAIRTECLIDGELKGFGQLHPRTQGVETLGTIYSSSLFPNRAPPGRILLLNYIGGSTNT
GILSKSEGELVEAVDRDLRKMLIKPNSTDPLKLGVRVWPQAIPQFLVGHFDILDTAKSSLTSS
GYEGLFLGGNYVAGVALGRCVEGAYETAIEVNNFMSRYAYK*
Arabidopsis thaliana PPO1 G404A (SEQ ID NO: 40)
MELSLLRPTTQSLLPSFSKPNLRLNVYKPLRLRCSVAGGPTVGSSKIEGGGGTTITTDCVIV
GGGISGLCIAQALATKHPDAAPNLIVTEAKDRVGGNIITREENGFLWEEGPNSFQPSDPMLT
MVVDSGLKDDLVLGDPTAPRFVLWNGKLRPVPSKLTDLPFFDLMSIGGKIRAGFGALGIRPS
PPGREESVEEFVRRNLGDEVFERLIEPFCSGVYAGDPSKLSMKAAFGKVWKLEQNGGSIIG
GTFKAIQERKNAPKAERDPRLPKPQGQTVGSFRKGLRMLPEAISARLGSKVKLSWKLSGIT
KLESGGYNLTYETPDGLVSVQSKSVVMTVPSHVASGLLRPLSESAANALSKLYYPPVAAVSI
SYPKEAIRTECLIDGELKGFGQLHPRTQGVETLATIYSSSLFPNRAPPGRILLLNYIGGSTNT
GILSKSEGELVEAVDRDLRKMLIKPNSTDPLKLGVRVWPQAIPQFLVGHFDILDTAKSSLTSS
GYEGLFLGGNYVAGVALGRCVEGAYETAIEVNNFMSRYAYK*
Arabidopsis thaliana PPO1 P413G (SEQ ID NO: 41)
MELSLLRPTTQSLLPSFSKPNLRLNVYKPLRLRCSVAGGPTVGSSKIEGGGGTTITTDCVIV
GGGISGLCIAQALATKHPDAAPNLIVTEAKDRVGGNIITREENGFLWEEGPNSFQPSDPMLT
MVVDSGLKDDLVLGDPTAPRFVLWNGKLRPVPSKLTDLPFFDLMSIGGKIRAGFGALGIRPS
PPGREESVEEFVRRNLGDEVFERLIEPFCSGVYAGDPSKLSMKAAFGKVWKLEQNGGSIIG
GTFKAIQERKNAPKAERDPRLPKPQGQTVGSFRKGLRMLPEAISARLGSKVKLSWKLSGIT
KLESGGYNLTYETPDGLVSVQSKSVVMTVPSHVASGLLRPLSESAANALSKLYYPPVAAVSI
SYPKEAIRTECLIDGELKGFGQLHPRTQGVETLGTIYSSSLFGNRAPPGRILLLNYIGGSTNT
GILSKSEGELVEAVDRDLRKMLIKPNSTDPLKLGVRVWPQAIPQFLVGHFDILDTAKSSLTSS
GYEGLFLGGNYVAGVALGRCVEGAYETAIEVNNFMSRYAYK*
Arabidopsis thaliana PPO1 P418M (SEQ ID NO: 42)
MELSLLRPTTQSLLPSFSKPNLRLNVYKPLRLRCSVAGGPTVGSSKIEGGGGTTITTDCVIV
GGGISGLCIAQALATKHPDAAPNLIVTEAKDRVGGNIITREENGFLWEEGPNSFQPSDPMLT
MVVDSGLKDDLVLGDPTAPRFVLWNGKLRPVPSKLTDLPFFDLMSIGGKIRAGFGALGIRPS
PPGREESVEEFVRRNLGDEVFERLIEPFCSGVYAGDPSKLSMKAAFGKVWKLEQNGGSIIG
GTFKAIQERKNAPKAERDPRLPKPQGQTVGSFRKGLRMLPEAISARLGSKVKLSWKLSGIT
KLESGGYNLTYETPDGLVSVQSKSVVMTVPSHVASGLLRPLSESAANALSKLYYPPVAAVSI
SYPKEAIRTECLIDGELKGFGQLHPRTQGVETLGTIYSSSLFPNRAPMGRILLLNYIGGSTNT
GILSKSEGELVEAVDRDLRKMLIKPNSTDPLKLGVRVWPQAIPQFLVGHFDILDTAKSSLTSS
GYEGLFLGGNYVAGVALGRCVEGAYETAIEVNNFMSRYAYK*
Arabidopsis thaliana PPO1 L424V (SEQ ID NO: 43)
MELSLLRPTTQSLLPSFSKPNLRLNVYKPLRLRCSVAGGPTVGSSKIEGGGGTTITTDCVIV
GGGISGLCIAQALATKHPDAAPNLIVTEAKDRVGGNIITREENGFLWEEGPNSFQPSDPMLT
MVVDSGLKDDLVLGDPTAPRFVLWNGKLRPVPSKLTDLPFFDLMSIGGKIRAGFGALGIRPS
PPGREESVEEFVRRNLGDEVFERLIEPFCSGVYAGDPSKLSMKAAFGKVWKLEQNGGSIIG
GTFKAIQERKNAPKAERDPRLPKPQGQTVGSFRKGLRMLPEAISARLGSKVKLSWKLSGIT
KLESGGYNLTYETPDGLVSVQSKSVVMTVPSHVASGLLRPLSESAANALSKLYYPPVAAVSI
SYPKEAIRTECLIDGELKGFGQLHPRTQGVETLGTIYSSSLFPNRAPPGRILLVNYIGGSTNT
GILSKSEGELVEAVDRDLRKMLIKPNSTDPLKLGVRVWPQAIPQFLVGHFDILDTAKSSLTSS
GYEGLFLGGNYVAGVALGRCVEGAYETAIEVNNFMSRYAYK*
Arabidopsis thaliana PPO1 Y426C (SEQ ID NO: 44)
MELSLLRPTTQSLLPSFSKPNLRLNVYKPLRLRCSVAGGPTVGSSKIEGGGGTTITTDCVIV
GGGISGLCIAQALATKHPDAAPNLIVTEAKDRVGGNIITREENGFLWEEGPNSFQPSDPMLT
MVVDSGLKDDLVLGDPTAPRFVLWNGKLRPVPSKLTDLPFFDLMSIGGKIRAGFGALGIRPS
PPGREESVEEFVRRNLGDEVFERLIEPFCSGVYAGDPSKLSMKAAFGKVWKLEQNGGSIIG
GTFKAIQERKNAPKAERDPRLPKPQGQTVGSFRKGLRMLPEAISARLGSKVKLSWKLSGIT
KLESGGYNLTYETPDGLVSVQSKSVVMTVPSHVASGLLRPLSESAANALSKLYYPPVAAVSI
SYPKEAIRTECLIDGELKGFGQLHPRTQGVETLGTIYSSSLFPNRAPPGRILLLNCIGGSTNT
GILSKSEGELVEAVDRDLRKMLIKPNSTDPLKLGVRVWPQAIPQFLVGHFDILDTAKSSLTSS
GYEGLFLGGNYVAGVALGRCVEGAYETAIEVNNFMSRYAYK*
Arabidopsis thaliana PPO1 Y426L (SEQ ID NO: 45)
MELSLLRPTTQSLLPSFSKPNLRLNVYKPLRLRCSVAGGPTVGSSKIEGGGGTTITTDCVIV
GGGISGLCIAQALATKHPDAAPNLIVTEAKDRVGGNIITREENGFLWEEGPNSFQPSDPMLT
MVVDSGLKDDLVLGDPTAPRFVLWNGKLRPVPSKLTDLPFFDLMSIGGKIRAGFGALGIRPS
PPGREESVEEFVRRNLGDEVFERLIEPFCSGVYAGDPSKLSMKAAFGKVWKLEQNGGSIIG
GTFKAIQERKNAPKAERDPRLPKPQGQTVGSFRKGLRMLPEAISARLGSKVKLSWKLSGIT
KLESGGYNLTYETPDGLVSVQSKSVVMTVPSHVASGLLRPLSESAANALSKLYYPPVAAVSI
SYPKEAIRTECLIDGELKGFGQLHPRTQGVETLGTIYSSSLFPNRAPPGRILLLNLIGGSTNT
GILSKSEGELVEAVDRDLRKMLIKPNSTDPLKLGVRVWPQAIPQFLVGHFDILDTAKSSLTSS
GYEGLFLGGNYVAGVALGRCVEGAYETAIEVNNFMSRYAYK*
Arabidopsis thaliana PPO1 Y426M (SEQ ID NO: 46)
MELSLLRPTTQSLLPSFSKPNLRLNVYKPLRLRCSVAGGPTVGSSKIEGGGGTTITTDCVIV
GGGISGLCIAQALATKHPDAAPNLIVTEAKDRVGGNIITREENGFLWEEGPNSFQPSDPMLT
MVVDSGLKDDLVLGDPTAPRFVLWNGKLRPVPSKLTDLPFFDLMSIGGKIRAGFGALGIRPS
PPGREESVEEFVRRNLGDEVFERLIEPFCSGVYAGDPSKLSMKAAFGKVWKLEQNGGSIIG
GTFKAIQERKNAPKAERDPRLPKPQGQTVGSFRKGLRMLPEAISARLGSKVKLSWKLSGIT
KLESGGYNLTYETPDGLVSVQSKSVVMTVPSHVASGLLRPLSESAANALSKLYYPPVAAVSI
SYPKEAIRTECLIDGELKGFGQLHPRTQGVETLGTIYSSSLFPNRAPPGRILLLNMIGGSTNT
GILSKSEGELVEAVDRDLRKMLIKPNSTDPLKLGVRVWPQAIPQFLVGHFDILDTAKSSLTSS
GYEGLFLGGNYVAGVALGRCVEGAYETAIEVNNFMSRYAYK*
Arabidopsis thaliana PPO1 Y426T (SEQ ID NO: 47)
MELSLLRPTTQSLLPSFSKPNLRLNVYKPLRLRCSVAGGPTVGSSKIEGGGGTTITTDCVIV
GGGISGLCIAQALATKHPDAAPNLIVTEAKDRVGGNIITREENGFLWEEGPNSFQPSDPMLT
MVVDSGLKDDLVLGDPTAPRFVLWNGKLRPVPSKLTDLPFFDLMSIGGKIRAGFGALGIRPS
PPGREESVEEFVRRNLGDEVFERLIEPFCSGVYAGDPSKLSMKAAFGKVWKLEQNGGSIIG
GTFKAIQERKNAPKAERDPRLPKPQGQTVGSFRKGLRMLPEAISARLGSKVKLSWKLSGIT
KLESGGYNLTYETPDGLVSVQSKSVVMTVPSHVASGLLRPLSESAANALSKLYYPPVAAVSI
SYPKEAIRTECLIDGELKGFGQLHPRTQGVETLGTIYSSSLFPNRAPPGRILLLNTIGGSTNT
GILSKSEGELVEAVDRDLRKMLIKPNSTDPLKLGVRVWPQAIPQFLVGHFDILDTAKSSLTSS
GYEGLFLGGNYVAGVALGRCVEGAYETAIEVNNFMSRYAYK*
Arabidopsis thaliana PPO1 Y426V (SEQ ID NO: 48)
MELSLLRPTTQSLLPSFSKPNLRLNVYKPLRLRCSVAGGPTVGSSKIEGGGGTTITTDCVIV
GGGISGLCIAQALATKHPDAAPNLIVTEAKDRVGGNIITREENGFLWEEGPNSFQPSDPMLT
MVVDSGLKDDLVLGDPTAPRFVLWNGKLRPVPSKLTDLPFFDLMSIGGKIRAGFGALGIRPS
PPGREESVEEFVRRNLGDEVFERLIEPFCSGVYAGDPSKLSMKAAFGKVWKLEQNGGSIIG
GTFKAIQERKNAPKAERDPRLPKPQGQTVGSFRKGLRMLPEAISARLGSKVKLSWKLSGIT
KLESGGYNLTYETPDGLVSVQSKSVVMTVPSHVASGLLRPLSESAANALSKLYYPPVAAVSI
SYPKEAIRTECLIDGELKGFGQLHPRTQGVETLGTIYSSSLFPNRAPPGRILLLNVIGGSTNT
GILSKSEGELVEAVDRDLRKMLIKPNSTDPLKLGVRVWPQAIPQFLVGHFDILDTAKSSLTSS
GYEGLFLGGNYVAGVALGRCVEGAYETAIEVNNFMSRYAYK*
Arabidopsis thaliana PPO1 T431R (SEQ ID NO: 49)
MELSLLRPTTQSLLPSFSKPNLRLNVYKPLRLRCSVAGGPTVGSSKIEGGGGTTITTDCVIV
GGGISGLCIAQALATKHPDAAPNLIVTEAKDRVGGNIITREENGFLWEEGPNSFQPSDPMLT
MVVDSGLKDDLVLGDPTAPRFVLWNGKLRPVPSKLTDLPFFDLMSIGGKIRAGFGALGIRPS
PPGREESVEEFVRRNLGDEVFERLIEPFCSGVYAGDPSKLSMKAAFGKVWKLEQNGGSIIG
GTFKAIQERKNAPKAERDPRLPKPQGQTVGSFRKGLRMLPEAISARLGSKVKLSWKLSGIT
KLESGGYNLTYETPDGLVSVQSKSVVMTVPSHVASGLLRPLSESAANALSKLYYPPVAAVSI
SYPKEAIRTECLIDGELKGFGQLHPRTQGVETLGTIYSSSLFPNRAPPGRILLLNYIGGSRNT
GILSKSEGELVEAVDRDLRKMLIKPNSTDPLKLGVRVWPQAIPQFLVGHFDILDTAKSSLTSS
GYEGLFLGGNYVAGVALGRCVEGAYETAIEVNNFMSRYAYK*
Arabidopsis thaliana PPO1 V447C (SEQ ID NO: 50)
MELSLLRPTTQSLLPSFSKPNLRLNVYKPLRLRCSVAGGPTVGSSKIEGGGGTTITTDCVIV
GGGISGLCIAQALATKHPDAAPNLIVTEAKDRVGGNIITREENGFLWEEGPNSFQPSDPMLT
MVVDSGLKDDLVLGDPTAPRFVLWNGKLRPVPSKLTDLPFFDLMSIGGKIRAGFGALGIRPS
PPGREESVEEFVRRNLGDEVFERLIEPFCSGVYAGDPSKLSMKAAFGKVWKLEQNGGSIIG
GTFKAIQERKNAPKAERDPRLPKPQGQTVGSFRKGLRMLPEAISARLGSKVKLSWKLSGIT
KLESGGYNLTYETPDGLVSVQSKSVVMTVPSHVASGLLRPLSESAANALSKLYYPPVAAVSI
SYPKEAIRTECLIDGELKGFGQLHPRTQGVETLGTIYSSSLFPNRAPPGRILLLNYIGGSTNT
GILSKSEGELVEACDRDLRKMLIKPNSTDPLKLGVRVWPQAIPQFLVGHFDILDTAKSSLTSS
GYEGLFLGGNYVAGVALGRCVEGAYETAIEVNNFMSRYAYK*
Arabidopsis thaliana PPO1 V447I (SEQ ID NO: 51)
MELSLLRPTTQSLLPSFSKPNLRLNVYKPLRLRCSVAGGPTVGSSKIEGGGGTTITTDCVIV
GGGISGLCIAQALATKHPDAAPNLIVTEAKDRVGGNIITREENGFLWEEGPNSFQPSDPMLT
MVVDSGLKDDLVLGDPTAPRFVLWNGKLRPVPSKLTDLPFFDLMSIGGKIRAGFGALGIRPS
PPGREESVEEFVRRNLGDEVFERLIEPFCSGVYAGDPSKLSMKAAFGKVWKLEQNGGSIIG
GTFKAIQERKNAPKAERDPRLPKPQGQTVGSFRKGLRMLPEAISARLGSKVKLSWKLSGIT
KLESGGYNLTYETPDGLVSVQSKSVVMTVPSHVASGLLRPLSESAANALSKLYYPPVAAVSI
SYPKEAIRTECLIDGELKGFGQLHPRTQGVETLGTIYSSSLFPNRAPPGRILLLNYIGGSTNT
GILSKSEGELVEAIDRDLRKMLIKPNSTDPLKLGVRVWPQAIPQFLVGHFDILDTAKSSLTSS
GYEGLFLGGNYVAGVALGRCVEGAYETAIEVNNFMSRYAYK*
Arabidopsis thaliana PPO1 D448H (SEQ ID NO: 52)
MELSLLRPTTQSLLPSFSKPNLRLNVYKPLRLRCSVAGGPTVGSSKIEGGGGTTITTDCVIV
GGGISGLCIAQALATKHPDAAPNLIVTEAKDRVGGNIITREENGFLWEEGPNSFQPSDPMLT
MVVDSGLKDDLVLGDPTAPRFVLWNGKLRPVPSKLTDLPFFDLMSIGGKIRAGFGALGIRPS
PPGREESVEEFVRRNLGDEVFERLIEPFCSGVYAGDPSKLSMKAAFGKVWKLEQNGGSIIG
GTFKAIQERKNAPKAERDPRLPKPQGQTVGSFRKGLRMLPEAISARLGSKVKLSWKLSGIT
KLESGGYNLTYETPDGLVSVQSKSVVMTVPSHVASGLLRPLSESAANALSKLYYPPVAAVSI
SYPKEAIRTECLIDGELKGFGQLHPRTQGVETLGTIYSSSLFPNRAPPGRILLLNYIGGSTNT
GILSKSEGELVEAVHRDLRKMLIKPNSTDPLKLGVRVWPQAIPQFLVGHFDILDTAKSSLTSS
GYEGLFLGGNYVAGVALGRCVEGAYETAIEVNNFMSRYAYK*
Arabidopsis thaliana PPO1 D448L (SEQ ID NO: 53)
MELSLLRPTTQSLLPSFSKPNLRLNVYKPLRLRCSVAGGPTVGSSKIEGGGGTTITTDCVIV
GGGISGLCIAQALATKHPDAAPNLIVTEAKDRVGGNIITREENGFLWEEGPNSFQPSDPMLT
MVVDSGLKDDLVLGDPTAPRFVLWNGKLRPVPSKLTDLPFFDLMSIGGKIRAGFGALGIRPS
PPGREESVEEFVRRNLGDEVFERLIEPFCSGVYAGDPSKLSMKAAFGKVWKLEQNGGSIIG
GTFKAIQERKNAPKAERDPRLPKPQGQTVGSFRKGLRMLPEAISARLGSKVKLSWKLSGIT
KLESGGYNLTYETPDGLVSVQSKSVVMTVPSHVASGLLRPLSESAANALSKLYYPPVAAVSI
SYPKEAIRTECLIDGELKGFGQLHPRTQGVETLGTIYSSSLFPNRAPPGRILLLNYIGGSTNT
GILSKSEGELVEAVLRDLRKMLIKPNSTDPLKLGVRVWPQAIPQFLVGHFDILDTAKSSLTSS
GYEGLFLGGNYVAGVALGRCVEGAYETAIEVNNFMSRYAYK*
Arabidopsis thaliana PPO1 D448N (SEQ ID NO: 54)
MELSLLRPTTQSLLPSFSKPNLRLNVYKPLRLRCSVAGGPTVGSSKIEGGGGTTITTDCVIV
GGGISGLCIAQALATKHPDAAPNLIVTEAKDRVGGNIITREENGFLWEEGPNSFQPSDPMLT
MVVDSGLKDDLVLGDPTAPRFVLWNGKLRPVPSKLTDLPFFDLMSIGGKIRAGFGALGIRPS
PPGREESVEEFVRRNLGDEVFERLIEPFCSGVYAGDPSKLSMKAAFGKVWKLEQNGGSIIG
GTFKAIQERKNAPKAERDPRLPKPQGQTVGSFRKGLRMLPEAISARLGSKVKLSWKLSGIT
KLESGGYNLTYETPDGLVSVQSKSVVMTVPSHVASGLLRPLSESAANALSKLYYPPVAAVSI
SYPKEAIRTECLIDGELKGFGQLHPRTQGVETLGTIYSSSLFPNRAPPGRILLLNYIGGSTNT
GILSKSEGELVEAVNRDLRKMLIKPNSTDPLKLGVRVWPQAIPQFLVGHFDILDTAKSSLTSS
GYEGLFLGGNYVAGVALGRCVEGAYETAIEVNNFMSRYAYK*
Arabidopsis thaliana PPO1 R449K (SEQ ID NO: 55)
MELSLLRPTTQSLLPSFSKPNLRLNVYKPLRLRCSVAGGPTVGSSKIEGGGGTTITTDCVIV
GGGISGLCIAQALATKHPDAAPNLIVTEAKDRVGGNIITREENGFLWEEGPNSFQPSDPMLT
MVVDSGLKDDLVLGDPTAPRFVLWNGKLRPVPSKLTDLPFFDLMSIGGKIRAGFGALGIRPS
PPGREESVEEFVRRNLGDEVFERLIEPFCSGVYAGDPSKLSMKAAFGKVWKLEQNGGSIIG
GTFKAIQERKNAPKAERDPRLPKPQGQTVGSFRKGLRMLPEAISARLGSKVKLSWKLSGIT
KLESGGYNLTYETPDGLVSVQSKSVVMTVPSHVASGLLRPLSESAANALSKLYYPPVAAVSI
SYPKEAIRTECLIDGELKGFGQLHPRTQGVETLGTIYSSSLFPNRAPPGRILLLNYIGGSTNT
GILSKSEGELVEAVDKDLRKMLIKPNSTDPLKLGVRVWPQAIPQFLVGHFDILDTAKSSLTSS
GYEGLFLGGNYVAGVALGRCVEGAYETAIEVNNFMSRYAYK*
Arabidopsis thaliana PPO1 T461Q (SEQ ID NO: 56)
MELSLLRPTTQSLLPSFSKPNLRLNVYKPLRLRCSVAGGPTVGSSKIEGGGGTTITTDCVIV
GGGISGLCIAQALATKHPDAAPNLIVTEAKDRVGGNIITREENGFLWEEGPNSFQPSDPMLT
MVVDSGLKDDLVLGDPTAPRFVLWNGKLRPVPSKLTDLPFFDLMSIGGKIRAGFGALGIRPS
PPGREESVEEFVRRNLGDEVFERLIEPFCSGVYAGDPSKLSMKAAFGKVWKLEQNGGSIIG
GTFKAIQERKNAPKAERDPRLPKPQGQTVGSFRKGLRMLPEAISARLGSKVKLSWKLSGIT
KLESGGYNLTYETPDGLVSVQSKSVVMTVPSHVASGLLRPLSESAANALSKLYYPPVAAVSI
SYPKEAIRTECLIDGELKGFGQLHPRTQGVETLGTIYSSSLFPNRAPPGRILLLNYIGGSTNT
GILSKSEGELVEAVDRDLRKMLIKPNSQDPLKLGVRVWPQAIPQFLVGHFDILDTAKSSLTSS
GYEGLFLGGNYVAGVALGRCVEGAYETAIEVNNFMSRYAYK*
Arabidopsis thaliana PPO1 F478R (SEQ ID NO: 57)
MELSLLRPTTQSLLPSFSKPNLRLNVYKPLRLRCSVAGGPTVGSSKIEGGGGTTITTDCVIV
GGGISGLCIAQALATKHPDAAPNLIVTEAKDRVGGNIITREENGFLWEEGPNSFQPSDPMLT
MVVDSGLKDDLVLGDPTAPRFVLWNGKLRPVPSKLTDLPFFDLMSIGGKIRAGFGALGIRPS
PPGREESVEEFVRRNLGDEVFERLIEPFCSGVYAGDPSKLSMKAAFGKVWKLEQNGGSIIG
GTFKAIQERKNAPKAERDPRLPKPQGQTVGSFRKGLRMLPEAISARLGSKVKLSWKLSGIT
KLESGGYNLTYETPDGLVSVQSKSVVMTVPSHVASGLLRPLSESAANALSKLYYPPVAAVSI
SYPKEAIRTECLIDGELKGFGQLHPRTQGVETLGTIYSSSLFPNRAPPGRILLLNYIGGSTNT
GILSKSEGELVEAVDRDLRKMLIKPNSTDPLKLGVRVWPQAIPQRLVGHFDILDTAKSSLTSS
GYEGLFLGGNYVAGVALGRCVEGAYETAIEVNNFMSRYAYK*
Arabidopsis thaliana PPO1 L479M (SEQ ID NO: 58)
MELSLLRPTTQSLLPSFSKPNLRLNVYKPLRLRCSVAGGPTVGSSKIEGGGGTTITTDCVIV
GGGISGLCIAQALATKHPDAAPNLIVTEAKDRVGGNIITREENGFLWEEGPNSFQPSDPMLT
MVVDSGLKDDLVLGDPTAPRFVLWNGKLRPVPSKLTDLPFFDLMSIGGKIRAGFGALGIRPS
PPGREESVEEFVRRNLGDEVFERLIEPFCSGVYAGDPSKLSMKAAFGKVWKLEQNGGSIIG
GTFKAIQERKNAPKAERDPRLPKPQGQTVGSFRKGLRMLPEAISARLGSKVKLSWKLSGIT
KLESGGYNLTYETPDGLVSVQSKSVVMTVPSHVASGLLRPLSESAANALSKLYYPPVAAVSI
SYPKEAIRTECLIDGELKGFGQLHPRTQGVETLGTIYSSSLFPNRAPPGRILLLNYIGGSTNT
GILSKSEGELVEAVDRDLRKMLIKPNSTDPLKLGVRVWPQAIPQFMVGHFDILDTAKSSLTS
SGYEGLFLGGNYVAGVALGRCVEGAYETAIEVNNFMSRYAYK*
Arabidopsis thaliana PPO1 L479N (SEQ ID NO: 59)
MELSLLRPTTQSLLPSFSKPNLRLNVYKPLRLRCSVAGGPTVGSSKIEGGGGTTITTDCVIV
GGGISGLCIAQALATKHPDAAPNLIVTEAKDRVGGNIITREENGFLWEEGPNSFQPSDPMLT
MVVDSGLKDDLVLGDPTAPRFVLWNGKLRPVPSKLTDLPFFDLMSIGGKIRAGFGALGIRPS
PPGREESVEEFVRRNLGDEVFERLIEPFCSGVYAGDPSKLSMKAAFGKVWKLEQNGGSIIG
GTFKAIQERKNAPKAERDPRLPKPQGQTVGSFRKGLRMLPEAISARLGSKVKLSWKLSGIT
KLESGGYNLTYETPDGLVSVQSKSVVMTVPSHVASGLLRPLSESAANALSKLYYPPVAAVSI
SYPKEAIRTECLIDGELKGFGQLHPRTQGVETLGTIYSSSLFPNRAPPGRILLLNYIGGSTNT
GILSKSEGELVEAVDRDLRKMLIKPNSTDPLKLGVRVWPQAIPQFNVGHFDILDTAKSSLTSS
GYEGLFLGGNYVAGVALGRCVEGAYETAIEVNNFMSRYAYK*
Arabidopsis thaliana PPO1 G510N (SEQ ID NO: 60)
MELSLLRPTTQSLLPSFSKPNLRLNVYKPLRLRCSVAGGPTVGSSKIEGGGGTTITTDCVIV
GGGISGLCIAQALATKHPDAAPNLIVTEAKDRVGGNIITREENGFLWEEGPNSFQPSDPMLT
MVVDSGLKDDLVLGDPTAPRFVLWNGKLRPVPSKLTDLPFFDLMSIGGKIRAGFGALGIRPS
PPGREESVEEFVRRNLGDEVFERLIEPFCSGVYAGDPSKLSMKAAFGKVWKLEQNGGSIIG
GTFKAIQERKNAPKAERDPRLPKPQGQTVGSFRKGLRMLPEAISARLGSKVKLSWKLSGIT
KLESGGYNLTYETPDGLVSVQSKSVVMTVPSHVASGLLRPLSESAANALSKLYYPPVAAVSI
SYPKEAIRTECLIDGELKGFGQLHPRTQGVETLGTIYSSSLFPNRAPPGRILLLNYIGGSTNT
GILSKSEGELVEAVDRDLRKMLIKPNSTDPLKLGVRVWPQAIPQFLVGHFDILDTAKSSLTSS
GYEGLFLGGNYVANVALGRCVEGAYETAIEVNNFMSRYAYK*
Arabidopsis thaliana PPO1 V511L (SEQ ID NO: 61)
MELSLLRPTTQSLLPSFSKPNLRLNVYKPLRLRCSVAGGPTVGSSKIEGGGGTTITTDCVIV
GGGISGLCIAQALATKHPDAAPNLIVTEAKDRVGGNIITREENGFLWEEGPNSFQPSDPMLT
MVVDSGLKDDLVLGDPTAPRFVLWNGKLRPVPSKLTDLPFFDLMSIGGKIRAGFGALGIRPS
PPGREESVEEFVRRNLGDEVFERLIEPFCSGVYAGDPSKLSMKAAFGKVWKLEQNGGSIIG
GTFKAIQERKNAPKAERDPRLPKPQGQTVGSFRKGLRMLPEAISARLGSKVKLSWKLSGIT
KLESGGYNLTYETPDGLVSVQSKSVVMTVPSHVASGLLRPLSESAANALSKLYYPPVAAVSI
SYPKEAIRTECLIDGELKGFGQLHPRTQGVETLGTIYSSSLFPNRAPPGRILLLNYIGGSTNT
GILSKSEGELVEAVDRDLRKMLIKPNSTDPLKLGVRVWPQAIPQFLVGHFDILDTAKSSLTSS
GYEGLFLGGNYVAGLALGRCVEGAYETAIEVNNFMSRYAYK*
Arabidopsis thaliana PPO1 V511N (SEQ ID NO: 62)
MELSLLRPTTQSLLPSFSKPNLRLNVYKPLRLRCSVAGGPTVGSSKIEGGGGTTITTDCVIV
GGGISGLCIAQALATKHPDAAPNLIVTEAKDRVGGNIITREENGFLWEEGPNSFQPSDPMLT
MVVDSGLKDDLVLGDPTAPRFVLWNGKLRPVPSKLTDLPFFDLMSIGGKIRAGFGALGIRPS
PPGREESVEEFVRRNLGDEVFERLIEPFCSGVYAGDPSKLSMKAAFGKVWKLEQNGGSIIG
GTFKAIQERKNAPKAERDPRLPKPQGQTVGSFRKGLRMLPEAISARLGSKVKLSWKLSGIT
KLESGGYNLTYETPDGLVSVQSKSVVMTVPSHVASGLLRPLSESAANALSKLYYPPVAAVSI
SYPKEAIRTECLIDGELKGFGQLHPRTQGVETLGTIYSSSLFPNRAPPGRILLLNYIGGSTNT
GILSKSEGELVEAVDRDLRKMLIKPNSTDPLKLGVRVWPQAIPQFLVGHFDILDTAKSSLTSS
GYEGLFLGGNYVAGNALGRCVEGAYETAIEVNNFMSRYAYK*
Arabidopsis thaliana PPO1 C516Q (SEQ ID NO: 63)
MELSLLRPTTQSLLPSFSKPNLRLNVYKPLRLRCSVAGGPTVGSSKIEGGGGTTITTDCVIV
GGGISGLCIAQALATKHPDAAPNLIVTEAKDRVGGNIITREENGFLWEEGPNSFQPSDPMLT
MVVDSGLKDDLVLGDPTAPRFVLWNGKLRPVPSKLTDLPFFDLMSIGGKIRAGFGALGIRPS
PPGREESVEEFVRRNLGDEVFERLIEPFCSGVYAGDPSKLSMKAAFGKVWKLEQNGGSIIG
GTFKAIQERKNAPKAERDPRLPKPQGQTVGSFRKGLRMLPEAISARLGSKVKLSWKLSGIT
KLESGGYNLTYETPDGLVSVQSKSVVMTVPSHVASGLLRPLSESAANALSKLYYPPVAAVSI
SYPKEAIRTECLIDGELKGFGQLHPRTQGVETLGTIYSSSLFPNRAPPGRILLLNYIGGSTNT
GILSKSEGELVEAVDRDLRKMLIKPNSTDPLKLGVRVWPQAIPQFLVGHFDILDTAKSSLTSS
GYEGLFLGGNYVAGVALGRQVEGAYETAIEVNNFMSRYAYK*
Setaria italica PPO1 G62A (SEQ ID NO: 64)
MVAAAMATAPSAGVPPLRGTRGPARFRIRGVSVRCAAVAGGAAEAPASAGARVSADCVVV
GAGISGLCTAQALATKHGVGDVLVTEARARPGGNITTVERPDEGYLWEEGPNSFQPSDPV
LTMAVDSGLKDDLVFGDPNAPRFVLWEGKLRPVPSKPADLPFFDLMSIPGKLRAGFGALGI
RPPPPGREESVEEFVRRNLGAEVFERLIEPFCSGVYAGDPSKLSMKAAFGKVWRLEEAGG
SIIGGTIKTIQERGKNPKPPRDPRLPTPKGQTVASFRKGLAMLPNAITSSLGSKVKLSWKLTSI
TKSDGMGYVLVYETPEGVVSVQAKSVIMTIPSYVASDILRPLSSDAADALSRFYYPPVAAVTI
SYPKEAIRKECLIDGELQGFGQLHPRSQGVETLGTIYSSSLFPNRAPAGRVLLLNYIGGATNT
GIVSKSASELVEAVDRDLRKMLINPSAVDPLVLGVRVWPQAIPQFLVGHLDLLEAAKSSLDR
GGYDGLFLGGNYVAGVALGRCVEGAYESASQISDFLTKYAYK*
Setaria italica PPO1 V84I (SEQ ID NO: 65)
MVAAAMATAPSAGVPPLRGTRGPARFRIRGVSVRCAAVAGGAAEAPASAGARVSADCVVV
GGGISGLCTAQALATKHGVGDVLITEARARPGGNITTVERPDEGYLWEEGPNSFQPSDPVL
TMAVDSGLKDDLVFGDPNAPRFVLWEGKLRPVPSKPADLPFFDLMSIPGKLRAGFGALGIR
PPPPGREESVEEFVRRNLGAEVFERLIEPFCSGVYAGDPSKLSMKAAFGKVWRLEEAGGSI
IGGTIKTIQERGKNPKPPRDPRLPTPKGQTVASFRKGLAMLPNAITSSLGSKVKLSWKLTSIT
KSDGMGYVLVYETPEGVVSVQAKSVIMTIPSYVASDILRPLSSDAADALSRFYYPPVAAVTIS
YPKEAIRKECLIDGELQGFGQLHPRSQGVETLGTIYSSSLFPNRAPAGRVLLLNYIGGATNT
GIVSKSASELVEAVDRDLRKMLINPSAVDPLVLGVRVWPQAIPQFLVGHLDLLEAAKSSLDR
GGYDGLFLGGNYVAGVALGRCVEGAYESASQISDFLTKYAYK*
Setaria italica PPO1 E109D (SEQ ID NO: 66)
MVAAAMATAPSAGVPPLRGTRGPARFRIRGVSVRCAAVAGGAAEAPASAGARVSADCVVV
GGGISGLCTAQALATKHGVGDVLVTEARARPGGNITTVERPDEGYLWEDGPNSFQPSDPV
LTMAVDSGLKDDLVFGDPNAPRFVLWEGKLRPVPSKPADLPFFDLMSIPGKLRAGFGALGI
RPPPPGREESVEEFVRRNLGAEVFERLIEPFCSGVYAGDPSKLSMKAAFGKVWRLEEAGG
SIIGGTIKTIQERGKNPKPPRDPRLPTPKGQTVASFRKGLAMLPNAITSSLGSKVKLSWKLTSI
TKSDGMGYVLVYETPEGVVSVQAKSVIMTIPSYVASDILRPLSSDAADALSRFYYPPVAAVTI
SYPKEAIRKECLIDGELQGFGQLHPRSQGVETLGTIYSSSLFPNRAPAGRVLLLNYIGGATNT
GIVSKSASELVEAVDRDLRKMLINPSAVDPLVLGVRVWPQAIPQFLVGHLDLLEAAKSSLDR
GGYDGLFLGGNYVAGVALGRCVEGAYESASQISDFLTKYAYK*
Setaria italica PPO1 E109F (SEQ ID NO: 67)
MVAAAMATAPSAGVPPLRGTRGPARFRIRGVSVRCAAVAGGAAEAPASAGARVSADCVVV
GGGISGLCTAQALATKHGVGDVLVTEARARPGGNITTVERPDEGYLWEFGPNSFQPSDPV
LTMAVDSGLKDDLVFGDPNAPRFVLWEGKLRPVPSKPADLPFFDLMSIPGKLRAGFGALGI
RPPPPGREESVEEFVRRNLGAEVFERLIEPFCSGVYAGDPSKLSMKAAFGKVWRLEEAGG
SIIGGTIKTIQERGKNPKPPRDPRLPTPKGQTVASFRKGLAMLPNAITSSLGSKVKLSWKLTSI
TKSDGMGYVLVYETPEGVVSVQAKSVIMTIPSYVASDILRPLSSDAADALSRFYYPPVAAVTI
SYPKEAIRKECLIDGELQGFGQLHPRSQGVETLGTIYSSSLFPNRAPAGRVLLLNYIGGATNT
GIVSKSASELVEAVDRDLRKMLINPSAVDPLVLGVRVWPQAIPQFLVGHLDLLEAAKSSLDR
GGYDGLFLGGNYVAGVALGRCVEGAYESASQISDFLTKYAYK*
Setaria italica PPO1 V125I (SEQ ID NO: 68)
MVAAAMATAPSAGVPPLRGTRGPARFRIRGVSVRCAAVAGGAAEAPASAGARVSADCVVV
GGGISGLCTAQALATKHGVGDVLVTEARARPGGNITTVERPDEGYLWEEGPNSFQPSDPV
LTMAIDSGLKDDLVFGDPNAPRFVLWEGKLRPVPSKPADLPFFDLMSIPGKLRAGFGALGIR
PPPPGREESVEEFVRRNLGAEVFERLIEPFCSGVYAGDPSKLSMKAAFGKVWRLEEAGGSI
IGGTIKTIQERGKNPKPPRDPRLPTPKGQTVASFRKGLAMLPNAITSSLGSKVKLSWKLTSIT
KSDGMGYVLVYETPEGVVSVQAKSVIMTIPSYVASDILRPLSSDAADALSRFYYPPVAAVTIS
YPKEAIRKECLIDGELQGFGQLHPRSQGVETLGTIYSSSLFPNRAPAGRVLLLNYIGGATNT
GIVSKSASELVEAVDRDLRKMLINPSAVDPLVLGVRVWPQAIPQFLVGHLDLLEAAKSSLDR
GGYDGLFLGGNYVAGVALGRCVEGAYESASQISDFLTKYAYK*
Setaria italica PPO1 V125L (SEQ ID NO: 69)
MVAAAMATAPSAGVPPLRGTRGPARFRIRGVSVRCAAVAGGAAEAPASAGARVSADCVVV
GGGISGLCTAQALATKHGVGDVLVTEARARPGGNITTVERPDEGYLWEEGPNSFQPSDPV
LTMALDSGLKDDLVFGDPNAPRFVLWEGKLRPVPSKPADLPFFDLMSIPGKLRAGFGALGI
RPPPPGREESVEEFVRRNLGAEVFERLIEPFCSGVYAGDPSKLSMKAAFGKVWRLEEAGG
SIIGGTIKTIQERGKNPKPPRDPRLPTPKGQTVASFRKGLAMLPNAITSSLGSKVKLSWKLTSI
TKSDGMGYVLVYETPEGVVSVQAKSVIMTIPSYVASDILRPLSSDAADALSRFYYPPVAAVTI
SYPKEAIRKECLIDGELQGFGQLHPRSQGVETLGTIYSSSLFPNRAPAGRVLLLNYIGGATNT
GIVSKSASELVEAVDRDLRKMLINPSAVDPLVLGVRVWPQAIPQFLVGHLDLLEAAKSSLDR
GGYDGLFLGGNYVAGVALGRCVEGAYESASQISDFLTKYAYK*
Setaria italica PPO1 P152Y (SEQ ID NO: 70)
MVAAAMATAPSAGVPPLRGTRGPARFRIRGVSVRCAAVAGGAAEAPASAGARVSADCVVV
GGGISGLCTAQALATKHGVGDVLVTEARARPGGNITTVERPDEGYLWEEGPNSFQPSDPV
LTMAVDSGLKDDLVFGDPNAPRFVLWEGKLRYVPSKPADLPFFDLMSIPGKLRAGFGALGI
RPPPPGREESVEEFVRRNLGAEVFERLIEPFCSGVYAGDPSKLSMKAAFGKVWRLEEAGG
SIIGGTIKTIQERGKNPKPPRDPRLPTPKGQTVASFRKGLAMLPNAITSSLGSKVKLSWKLTSI
TKSDGMGYVLVYETPEGVVSVQAKSVIMTIPSYVASDILRPLSSDAADALSRFYYPPVAAVTI
SYPKEAIRKECLIDGELQGFGQLHPRSQGVETLGTIYSSSLFPNRAPAGRVLLLNYIGGATNT
GIVSKSASELVEAVDRDLRKMLINPSAVDPLVLGVRVWPQAIPQFLVGHLDLLEAAKSSLDR
GGYDGLFLGGNYVAGVALGRCVEGAYESASQISDFLTKYAYK*
Setaria italica PPO1 K171H (SEQ ID NO: 71)
MVAAAMATAPSAGVPPLRGTRGPARFRIRGVSVRCAAVAGGAAEAPASAGARVSADCVVV
GGGISGLCTAQALATKHGVGDVLVTEARARPGGNITTVERPDEGYLWEEGPNSFQPSDPV
LTMAVDSGLKDDLVFGDPNAPRFVLWEGKLRPVPSKPADLPFFDLMSIPGHLRAGFGALGI
RPPPPGREESVEEFVRRNLGAEVFERLIEPFCSGVYAGDPSKLSMKAAFGKVWRLEEAGG
SIIGGTIKTIQERGKNPKPPRDPRLPTPKGQTVASFRKGLAMLPNAITSSLGSKVKLSWKLTSI
TKSDGMGYVLVYETPEGVVSVQAKSVIMTIPSYVASDILRPLSSDAADALSRFYYPPVAAVTI
SYPKEAIRKECLIDGELQGFGQLHPRSQGVETLGTIYSSSLFPNRAPAGRVLLLNYIGGATNT
GIVSKSASELVEAVDRDLRKMLINPSAVDPLVLGVRVWPQAIPQFLVGHLDLLEAAKSSLDR
GGYDGLFLGGNYVAGVALGRCVEGAYESASQISDFLTKYAYK*
Setaria italica PPO1 A174L (SEQ ID NO: 72)
MVAAAMATAPSAGVPPLRGTRGPARFRIRGVSVRCAAVAGGAAEAPASAGARVSADCVVV
GGGISGLCTAQALATKHGVGDVLVTEARARPGGNITTVERPDEGYLWEEGPNSFQPSDPV
LTMAVDSGLKDDLVFGDPNAPRFVLWEGKLRPVPSKPADLPFFDLMSIPGKLRLGFGALGI
RPPPPGREESVEEFVRRNLGAEVFERLIEPFCSGVYAGDPSKLSMKAAFGKVWRLEEAGG
SIIGGTIKTIQERGKNPKPPRDPRLPTPKGQTVASFRKGLAMLPNAITSSLGSKVKLSWKLTSI
TKSDGMGYVLVYETPEGVVSVQAKSVIMTIPSYVASDILRPLSSDAADALSRFYYPPVAAVTI
SYPKEAIRKECLIDGELQGFGQLHPRSQGVETLGTIYSSSLFPNRAPAGRVLLLNYIGGATNT
GIVSKSASELVEAVDRDLRKMLINPSAVDPLVLGVRVWPQAIPQFLVGHLDLLEAAKSSLDR
GGYDGLFLGGNYVAGVALGRCVEGAYESASQISDFLTKYAYK*
Setaria italica PPO1 V196W (SEQ ID NO: 73)
MVAAAMATAPSAGVPPLRGTRGPARFRIRGVSVRCAAVAGGAAEAPASAGARVSADCVVV
GGGISGLCTAQALATKHGVGDVLVTEARARPGGNITTVERPDEGYLWEEGPNSFQPSDPV
LTMAVDSGLKDDLVFGDPNAPRFVLWEGKLRPVPSKPADLPFFDLMSIPGKLRAGFGALGI
RPPPPGREESVEEFWRRNLGAEVFERLIEPFCSGVYAGDPSKLSMKAAFGKVWRLEEAGG
SIIGGTIKTIQERGKNPKPPRDPRLPTPKGQTVASFRKGLAMLPNAITSSLGSKVKLSWKLTSI
TKSDGMGYVLVYETPEGVVSVQAKSVIMTIPSYVASDILRPLSSDAADALSRFYYPPVAAVTI
SYPKEAIRKECLIDGELQGFGQLHPRSQGVETLGTIYSSSLFPNRAPAGRVLLLNYIGGATNT
GIVSKSASELVEAVDRDLRKMLINPSAVDPLVLGVRVWPQAIPQFLVGHLDLLEAAKSSLDR
GGYDGLFLGGNYVAGVALGRCVEGAYESASQISDFLTKYAYK*
Setaria italica PPO1 V204G (SEQ ID NO: 74)
MVAAAMATAPSAGVPPLRGTRGPARFRIRGVSVRCAAVAGGAAEAPASAGARVSADCVVV
GGGISGLCTAQALATKHGVGDVLVTEARARPGGNITTVERPDEGYLWEEGPNSFQPSDPV
LTMAVDSGLKDDLVFGDPNAPRFVLWEGKLRPVPSKPADLPFFDLMSIPGKLRAGFGALGI
RPPPPGREESVEEFVRRNLGAEGFERLIEPFCSGVYAGDPSKLSMKAAFGKVWRLEEAGG
SIIGGTIKTIQERGKNPKPPRDPRLPTPKGQTVASFRKGLAMLPNAITSSLGSKVKLSWKLTSI
TKSDGMGYVLVYETPEGVVSVQAKSVIMTIPSYVASDILRPLSSDAADALSRFYYPPVAAVTI
SYPKEAIRKECLIDGELQGFGQLHPRSQGVETLGTIYSSSLFPNRAPAGRVLLLNYIGGATNT
GIVSKSASELVEAVDRDLRKMLINPSAVDPLVLGVRVWPQAIPQFLVGHLDLLEAAKSSLDR
GGYDGLFLGGNYVAGVALGRCVEGAYESASQISDFLTKYAYK*
Setaria italica PPO1 F205Y (SEQ ID NO: 75)
MVAAAMATAPSAGVPPLRGTRGPARFRIRGVSVRCAAVAGGAAEAPASAGARVSADCVVV
GGGISGLCTAQALATKHGVGDVLVTEARARPGGNITTVERPDEGYLWEEGPNSFQPSDPV
LTMAVDSGLKDDLVFGDPNAPRFVLWEGKLRPVPSKPADLPFFDLMSIPGKLRAGFGALGI
RPPPPGREESVEEFVRRNLGAEVYERLIEPFCSGVYAGDPSKLSMKAAFGKVWRLEEAGG
SIIGGTIKTIQERGKNPKPPRDPRLPTPKGQTVASFRKGLAMLPNAITSSLGSKVKLSWKLTSI
TKSDGMGYVLVYETPEGVVSVQAKSVIMTIPSYVASDILRPLSSDAADALSRFYYPPVAAVTI
SYPKEAIRKECLIDGELQGFGQLHPRSQGVETLGTIYSSSLFPNRAPAGRVLLLNYIGGATNT
GIVSKSASELVEAVDRDLRKMLINPSAVDPLVLGVRVWPQAIPQFLVGHLDLLEAAKSSLDR
GGYDGLFLGGNYVAGVALGRCVEGAYESASQISDFLTKYAYK*
Setaria italica PPO1 E210Y (SEQ ID NO: 76)
MVAAAMATAPSAGVPPLRGTRGPARFRIRGVSVRCAAVAGGAAEAPASAGARVSADCVVV
GGGISGLCTAQALATKHGVGDVLVTEARARPGGNITTVERPDEGYLWEEGPNSFQPSDPV
LTMAVDSGLKDDLVFGDPNAPRFVLWEGKLRPVPSKPADLPFFDLMSIPGKLRAGFGALGI
RPPPPGREESVEEFVRRNLGAEVFERLIYPFCSGVYAGDPSKLSMKAAFGKVWRLEEAGG
SIIGGTIKTIQERGKNPKPPRDPRLPTPKGQTVASFRKGLAMLPNAITSSLGSKVKLSWKLTSI
TKSDGMGYVLVYETPEGVVSVQAKSVIMTIPSYVASDILRPLSSDAADALSRFYYPPVAAVTI
SYPKEAIRKECLIDGELQGFGQLHPRSQGVETLGTIYSSSLFPNRAPAGRVLLLNYIGGATNT
GIVSKSASELVEAVDRDLRKMLINPSAVDPLVLGVRVWPQAIPQFLVGHLDLLEAAKSSLDR
GGYDGLFLGGNYVAGVALGRCVEGAYESASQISDFLTKYAYK*
Setaria italica PPO1 F212Y (SEQ ID NO: 77)
MVAAAMATAPSAGVPPLRGTRGPARFRIRGVSVRCAAVAGGAAEAPASAGARVSADCVVV
GGGISGLCTAQALATKHGVGDVLVTEARARPGGNITTVERPDEGYLWEEGPNSFQPSDPV
LTMAVDSGLKDDLVFGDPNAPRFVLWEGKLRPVPSKPADLPFFDLMSIPGKLRAGFGALGI
RPPPPGREESVEEFVRRNLGAEVFERLIEPYCSGVYAGDPSKLSMKAAFGKVWRLEEAGG
SIIGGTIKTIQERGKNPKPPRDPRLPTPKGQTVASFRKGLAMLPNAITSSLGSKVKLSWKLTSI
TKSDGMGYVLVYETPEGVVSVQAKSVIMTIPSYVASDILRPLSSDAADALSRFYYPPVAAVTI
SYPKEAIRKECLIDGELQGFGQLHPRSQGVETLGTIYSSSLFPNRAPAGRVLLLNYIGGATNT
GIVSKSASELVEAVDRDLRKMLINPSAVDPLVLGVRVWPQAIPQFLVGHLDLLEAAKSSLDR
GGYDGLFLGGNYVAGVALGRCVEGAYESASQISDFLTKYAYK*
Setaria italica PPO1 V216K (SEQ ID NO: 78)
MVAAAMATAPSAGVPPLRGTRGPARFRIRGVSVRCAAVAGGAAEAPASAGARVSADCVVV
GGGISGLCTAQALATKHGVGDVLVTEARARPGGNITTVERPDEGYLWEEGPNSFQPSDPV
LTMAVDSGLKDDLVFGDPNAPRFVLWEGKLRPVPSKPADLPFFDLMSIPGKLRAGFGALGI
RPPPPGREESVEEFVRRNLGAEVFERLIEPFCSGKYAGDPSKLSMKAAFGKVWRLEEAGG
SIIGGTIKTIQERGKNPKPPRDPRLPTPKGQTVASFRKGLAMLPNAITSSLGSKVKLSWKLTSI
TKSDGMGYVLVYETPEGVVSVQAKSVIMTIPSYVASDILRPLSSDAADALSRFYYPPVAAVTI
SYPKEAIRKECLIDGELQGFGQLHPRSQGVETLGTIYSSSLFPNRAPAGRVLLLNYIGGATNT
GIVSKSASELVEAVDRDLRKMLINPSAVDPLVLGVRVWPQAIPQFLVGHLDLLEAAKSSLDR
GGYDGLFLGGNYVAGVALGRCVEGAYESASQISDFLTKYAYK*
Setaria italica PPO1 Y217S (SEQ ID NO: 79)
MVAAAMATAPSAGVPPLRGTRGPARFRIRGVSVRCAAVAGGAAEAPASAGARVSADCVVV
GGGISGLCTAQALATKHGVGDVLVTEARARPGGNITTVERPDEGYLWEEGPNSFQPSDPV
LTMAVDSGLKDDLVFGDPNAPRFVLWEGKLRPVPSKPADLPFFDLMSIPGKLRAGFGALGI
RPPPPGREESVEEFVRRNLGAEVFERLIEPFCSGVSAGDPSKLSMKAAFGKVWRLEEAGG
SIIGGTIKTIQERGKNPKPPRDPRLPTPKGQTVASFRKGLAMLPNAITSSLGSKVKLSWKLTSI
TKSDGMGYVLVYETPEGVVSVQAKSVIMTIPSYVASDILRPLSSDAADALSRFYYPPVAAVTI
SYPKEAIRKECLIDGELQGFGQLHPRSQGVETLGTIYSSSLFPNRAPAGRVLLLNYIGGATNT
GIVSKSASELVEAVDRDLRKMLINPSAVDPLVLGVRVWPQAIPQFLVGHLDLLEAAKSSLDR
GGYDGLFLGGNYVAGVALGRCVEGAYESASQISDFLTKYAYK*
Setaria italica PPO1 A218C (SEQ ID NO: 80)
MVAAAMATAPSAGVPPLRGTRGPARFRIRGVSVRCAAVAGGAAEAPASAGARVSADCVVV
GGGISGLCTAQALATKHGVGDVLVTEARARPGGNITTVERPDEGYLWEEGPNSFQPSDPV
LTMAVDSGLKDDLVFGDPNAPRFVLWEGKLRPVPSKPADLPFFDLMSIPGKLRAGFGALGI
RPPPPGREESVEEFVRRNLGAEVFERLIEPFCSGVYCGDPSKLSMKAAFGKVWRLEEAGG
SIIGGTIKTIQERGKNPKPPRDPRLPTPKGQTVASFRKGLAMLPNAITSSLGSKVKLSWKLTSI
TKSDGMGYVLVYETPEGVVSVQAKSVIMTIPSYVASDILRPLSSDAADALSRFYYPPVAAVTI
SYPKEAIRKECLIDGELQGFGQLHPRSQGVETLGTIYSSSLFPNRAPAGRVLLLNYIGGATNT
GIVSKSASELVEAVDRDLRKMLINPSAVDPLVLGVRVWPQAIPQFLVGHLDLLEAAKSSLDR
GGYDGLFLGGNYVAGVALGRCVEGAYESASQISDFLTKYAYK*
Setaria italica PPO1 P269C (SEQ ID NO: 81)
MVAAAMATAPSAGVPPLRGTRGPARFRIRGVSVRCAAVAGGAAEAPASAGARVSADCVVV
GGGISGLCTAQALATKHGVGDVLVTEARARPGGNITTVERPDEGYLWEEGPNSFQPSDPV
LTMAVDSGLKDDLVFGDPNAPRFVLWEGKLRPVPSKPADLPFFDLMSIPGKLRAGFGALGI
RPPPPGREESVEEFVRRNLGAEVFERLIEPFCSGVYAGDPSKLSMKAAFGKVWRLEEAGG
SIIGGTIKTIQERGKNPKPPRDPRLPTCKGQTVASFRKGLAMLPNAITSSLGSKVKLSWKLTS
ITKSDGMGYVLVYETPEGVVSVQAKSVIMTIPSYVASDILRPLSSDAADALSRFYYPPVAAVT
ISYPKEAIRKECLIDGELQGFGQLHPRSQGVETLGTIYSSSLFPNRAPAGRVLLLNYIGGATN
TGIVSKSASELVEAVDRDLRKMLINPSAVDPLVLGVRVWPQAIPQFLVGHLDLLEAAKSSLD
RGGYDGLFLGGNYVAGVALGRCVEGAYESASQISDFLTKYAYK*
Setaria italica PPO1 K270H (SEQ ID NO: 82)
MVAAAMATAPSAGVPPLRGTRGPARFRIRGVSVRCAAVAGGAAEAPASAGARVSADCVVV
GGGISGLCTAQALATKHGVGDVLVTEARARPGGNITTVERPDEGYLWEEGPNSFQPSDPV
LTMAVDSGLKDDLVFGDPNAPRFVLWEGKLRPVPSKPADLPFFDLMSIPGKLRAGFGALGI
RPPPPGREESVEEFVRRNLGAEVFERLIEPFCSGVYAGDPSKLSMKAAFGKVWRLEEAGG
SIIGGTIKTIQERGKNPKPPRDPRLPTPHGQTVASFRKGLAMLPNAITSSLGSKVKLSWKLTS
ITKSDGMGYVLVYETPEGVVSVQAKSVIMTIPSYVASDILRPLSSDAADALSRFYYPPVAAVT
ISYPKEAIRKECLIDGELQGFGQLHPRSQGVETLGTIYSSSLFPNRAPAGRVLLLNYIGGATN
TGIVSKSASELVEAVDRDLRKMLINPSAVDPLVLGVRVWPQAIPQFLVGHLDLLEAAKSSLD
RGGYDGLFLGGNYVAGVALGRCVEGAYESASQISDFLTKYAYK*
Setaria italica PPO1 K270I (SEQ ID NO: 83)
MVAAAMATAPSAGVPPLRGTRGPARFRIRGVSVRCAAVAGGAAEAPASAGARVSADCVVV
GGGISGLCTAQALATKHGVGDVLVTEARARPGGNITTVERPDEGYLWEEGPNSFQPSDPV
LTMAVDSGLKDDLVFGDPNAPRFVLWEGKLRPVPSKPADLPFFDLMSIPGKLRAGFGALGI
RPPPPGREESVEEFVRRNLGAEVFERLIEPFCSGVYAGDPSKLSMKAAFGKVWRLEEAGG
SIIGGTIKTIQERGKNPKPPRDPRLPTPIGQTVASFRKGLAMLPNAITSSLGSKVKLSWKLTSI
TKSDGMGYVLVYETPEGVVSVQAKSVIMTIPSYVASDILRPLSSDAADALSRFYYPPVAAVTI
SYPKEAIRKECLIDGELQGFGQLHPRSQGVETLGTIYSSSLFPNRAPAGRVLLLNYIGGATNT
GIVSKSASELVEAVDRDLRKMLINPSAVDPLVLGVRVWPQAIPQFLVGHLDLLEAAKSSLDR
GGYDGLFLGGNYVAGVALGRCVEGAYESASQISDFLTKYAYK*
Setaria italica PPO1 K270T (SEQ ID NO: 84)
MVAAAMATAPSAGVPPLRGTRGPARFRIRGVSVRCAAVAGGAAEAPASAGARVSADCVVV
GGGISGLCTAQALATKHGVGDVLVTEARARPGGNITTVERPDEGYLWEEGPNSFQPSDPV
LTMAVDSGLKDDLVFGDPNAPRFVLWEGKLRPVPSKPADLPFFDLMSIPGKLRAGFGALGI
RPPPPGREESVEEFVRRNLGAEVFERLIEPFCSGVYAGDPSKLSMKAAFGKVWRLEEAGG
SIIGGTIKTIQERGKNPKPPRDPRLPTPTGQTVASFRKGLAMLPNAITSSLGSKVKLSWKLTSI
TKSDGMGYVLVYETPEGVVSVQAKSVIMTIPSYVASDILRPLSSDAADALSRFYYPPVAAVTI
SYPKEAIRKECLIDGELQGFGQLHPRSQGVETLGTIYSSSLFPNRAPAGRVLLLNYIGGATNT
GIVSKSASELVEAVDRDLRKMLINPSAVDPLVLGVRVWPQAIPQFLVGHLDLLEAAKSSLDR
GGYDGLFLGGNYVAGVALGRCVEGAYESASQISDFLTKYAYK*
Setaria italica PPO1 K279C (SEQ ID NO: 85)
MVAAAMATAPSAGVPPLRGTRGPARFRIRGVSVRCAAVAGGAAEAPASAGARVSADCVVV
GGGISGLCTAQALATKHGVGDVLVTEARARPGGNITTVERPDEGYLWEEGPNSFQPSDPV
LTMAVDSGLKDDLVFGDPNAPRFVLWEGKLRPVPSKPADLPFFDLMSIPGKLRAGFGALGI
RPPPPGREESVEEFVRRNLGAEVFERLIEPFCSGVYAGDPSKLSMKAAFGKVWRLEEAGG
SIIGGTIKTIQERGKNPKPPRDPRLPTPKGQTVASFRCGLAMLPNAITSSLGSKVKLSWKLTS
ITKSDGMGYVLVYETPEGVVSVQAKSVIMTIPSYVASDILRPLSSDAADALSRFYYPPVAAVT
ISYPKEAIRKECLIDGELQGFGQLHPRSQGVETLGTIYSSSLFPNRAPAGRVLLLNYIGGATN
TGIVSKSASELVEAVDRDLRKMLINPSAVDPLVLGVRVWPQAIPQFLVGHLDLLEAAKSSLD
RGGYDGLFLGGNYVAGVALGRCVEGAYESASQISDFLTKYAYK*
Setaria italica PPO1 K279E (SEQ ID NO: 86)
MVAAAMATAPSAGVPPLRGTRGPARFRIRGVSVRCAAVAGGAAEAPASAGARVSADCVVV
GGGISGLCTAQALATKHGVGDVLVTEARARPGGNITTVERPDEGYLWEEGPNSFQPSDPV
LTMAVDSGLKDDLVFGDPNAPRFVLWEGKLRPVPSKPADLPFFDLMSIPGKLRAGFGALGI
RPPPPGREESVEEFVRRNLGAEVFERLIEPFCSGVYAGDPSKLSMKAAFGKVWRLEEAGG
SIIGGTIKTIQERGKNPKPPRDPRLPTPKGQTVASFREGLAMLPNAITSSLGSKVKLSWKLTSI
TKSDGMGYVLVYETPEGVVSVQAKSVIMTIPSYVASDILRPLSSDAADALSRFYYPPVAAVTI
SYPKEAIRKECLIDGELQGFGQLHPRSQGVETLGTIYSSSLFPNRAPAGRVLLLNYIGGATNT
GIVSKSASELVEAVDRDLRKMLINPSAVDPLVLGVRVWPQAIPQFLVGHLDLLEAAKSSLDR
GGYDGLFLGGNYVAGVALGRCVEGAYESASQISDFLTKYAYK*
Setaria italica PPO1 K279N (SEQ ID NO: 87)
MVAAAMATAPSAGVPPLRGTRGPARFRIRGVSVRCAAVAGGAAEAPASAGARVSADCVVV
GGGISGLCTAQALATKHGVGDVLVTEARARPGGNITTVERPDEGYLWEEGPNSFQPSDPV
LTMAVDSGLKDDLVFGDPNAPRFVLWEGKLRPVPSKPADLPFFDLMSIPGKLRAGFGALGI
RPPPPGREESVEEFVRRNLGAEVFERLIEPFCSGVYAGDPSKLSMKAAFGKVWRLEEAGG
SIIGGTIKTIQERGKNPKPPRDPRLPTPKGQTVASFRNGLAMLPNAITSSLGSKVKLSWKLTS
ITKSDGMGYVLVYETPEGVVSVQAKSVIMTIPSYVASDILRPLSSDAADALSRFYYPPVAAVT
ISYPKEAIRKECLIDGELQGFGQLHPRSQGVETLGTIYSSSLFPNRAPAGRVLLLNYIGGATN
TGIVSKSASELVEAVDRDLRKMLINPSAVDPLVLGVRVWPQAIPQFLVGHLDLLEAAKSSLD
RGGYDGLFLGGNYVAGVALGRCVEGAYESASQISDFLTKYAYK*
Setaria italica PPO1 A282G (SEQ ID NO: 88)
MVAAAMATAPSAGVPPLRGTRGPARFRIRGVSVRCAAVAGGAAEAPASAGARVSADCVVV
GGGISGLCTAQALATKHGVGDVLVTEARARPGGNITTVERPDEGYLWEEGPNSFQPSDPV
LTMAVDSGLKDDLVFGDPNAPRFVLWEGKLRPVPSKPADLPFFDLMSIPGKLRAGFGALGI
RPPPPGREESVEEFVRRNLGAEVFERLIEPFCSGVYAGDPSKLSMKAAFGKVWRLEEAGG
SIIGGTIKTIQERGKNPKPPRDPRLPTPKGQTVASFRKGLGMLPNAITSSLGSKVKLSWKLTS
ITKSDGMGYVLVYETPEGVVSVQAKSVIMTIPSYVASDILRPLSSDAADALSRFYYPPVAAVT
ISYPKEAIRKECLIDGELQGFGQLHPRSQGVETLGTIYSSSLFPNRAPAGRVLLLNYIGGATN
TGIVSKSASELVEAVDRDLRKMLINPSAVDPLVLGVRVWPQAIPQFLVGHLDLLEAAKSSLD
RGGYDGLFLGGNYVAGVALGRCVEGAYESASQISDFLTKYAYK*
Setaria italica PPO1 T303L (SEQ ID NO: 89)
MVAAAMATAPSAGVPPLRGTRGPARFRIRGVSVRCAAVAGGAAEAPASAGARVSADCVVV
GGGISGLCTAQALATKHGVGDVLVTEARARPGGNITTVERPDEGYLWEEGPNSFQPSDPV
LTMAVDSGLKDDLVFGDPNAPRFVLWEGKLRPVPSKPADLPFFDLMSIPGKLRAGFGALGI
RPPPPGREESVEEFVRRNLGAEVFERLIEPFCSGVYAGDPSKLSMKAAFGKVWRLEEAGG
SIIGGTIKTIQERGKNPKPPRDPRLPTPKGQTVASFRKGLAMLPNAITSSLGSKVKLSWKLLSI
TKSDGMGYVLVYETPEGVVSVQAKSVIMTIPSYVASDILRPLSSDAADALSRFYYPPVAAVTI
SYPKEAIRKECLIDGELQGFGQLHPRSQGVETLGTIYSSSLFPNRAPAGRVLLLNYIGGATNT
GIVSKSASELVEAVDRDLRKMLINPSAVDPLVLGVRVWPQAIPQFLVGHLDLLEAAKSSLDR
GGYDGLFLGGNYVAGVALGRCVEGAYESASQISDFLTKYAYK*
Setaria italica PPO1 F358C (SEQ ID NO: 90)
MVAAAMATAPSAGVPPLRGTRGPARFRIRGVSVRCAAVAGGAAEAPASAGARVSADCVVV
GGGISGLCTAQALATKHGVGDVLVTEARARPGGNITTVERPDEGYLWEEGPNSFQPSDPV
LTMAVDSGLKDDLVFGDPNAPRFVLWEGKLRPVPSKPADLPFFDLMSIPGKLRAGFGALGI
RPPPPGREESVEEFVRRNLGAEVFERLIEPFCSGVYAGDPSKLSMKAAFGKVWRLEEAGG
SIIGGTIKTIQERGKNPKPPRDPRLPTPKGQTVASFRKGLAMLPNAITSSLGSKVKLSWKLTSI
TKSDGMGYVLVYETPEGVVSVQAKSVIMTIPSYVASDILRPLSSDAADALSRCYYPPVAAVTI
SYPKEAIRKECLIDGELQGFGQLHPRSQGVETLGTIYSSSLFPNRAPAGRVLLLNYIGGATNT
GIVSKSASELVEAVDRDLRKMLINPSAVDPLVLGVRVWPQAIPQFLVGHLDLLEAAKSSLDR
GGYDGLFLGGNYVAGVALGRCVEGAYESASQISDFLTKYAYK*
Setaria italica PPO1 Y359D (SEQ ID NO: 91)
MVAAAMATAPSAGVPPLRGTRGPARFRIRGVSVRCAAVAGGAAEAPASAGARVSADCVVV
GGGISGLCTAQALATKHGVGDVLVTEARARPGGNITTVERPDEGYLWEEGPNSFQPSDPV
LTMAVDSGLKDDLVFGDPNAPRFVLWEGKLRPVPSKPADLPFFDLMSIPGKLRAGFGALGI
RPPPPGREESVEEFVRRNLGAEVFERLIEPFCSGVYAGDPSKLSMKAAFGKVWRLEEAGG
SIIGGTIKTIQERGKNPKPPRDPRLPTPKGQTVASFRKGLAMLPNAITSSLGSKVKLSWKLTSI
TKSDGMGYVLVYETPEGVVSVQAKSVIMTIPSYVASDILRPLSSDAADALSRFDYPPVAAVTI
SYPKEAIRKECLIDGELQGFGQLHPRSQGVETLGTIYSSSLFPNRAPAGRVLLLNYIGGATNT
GIVSKSASELVEAVDRDLRKMLINPSAVDPLVLGVRVWPQAIPQFLVGHLDLLEAAKSSLDR
GGYDGLFLGGNYVAGVALGRCVEGAYESASQISDFLTKYAYK*
Setaria italica PPO1 Y359F (SEQ ID NO: 92)
MVAAAMATAPSAGVPPLRGTRGPARFRIRGVSVRCAAVAGGAAEAPASAGARVSADCVVV
GGGISGLCTAQALATKHGVGDVLVTEARARPGGNITTVERPDEGYLWEEGPNSFQPSDPV
LTMAVDSGLKDDLVFGDPNAPRFVLWEGKLRPVPSKPADLPFFDLMSIPGKLRAGFGALGI
RPPPPGREESVEEFVRRNLGAEVFERLIEPFCSGVYAGDPSKLSMKAAFGKVWRLEEAGG
SIIGGTIKTIQERGKNPKPPRDPRLPTPKGQTVASFRKGLAMLPNAITSSLGSKVKLSWKLTSI
TKSDGMGYVLVYETPEGVVSVQAKSVIMTIPSYVASDILRPLSSDAADALSRFFYPPVAAVTI
SYPKEAIRKECLIDGELQGFGQLHPRSQGVETLGTIYSSSLFPNRAPAGRVLLLNYIGGATNT
GIVSKSASELVEAVDRDLRKMLINPSAVDPLVLGVRVWPQAIPQFLVGHLDLLEAAKSSLDR
GGYDGLFLGGNYVAGVALGRCVEGAYESASQISDFLTKYAYK*
Setaria italica PPO1 Y359P (SEQ ID NO: 93)
MVAAAMATAPSAGVPPLRGTRGPARFRIRGVSVRCAAVAGGAAEAPASAGARVSADCVVV
GGGISGLCTAQALATKHGVGDVLVTEARARPGGNITTVERPDEGYLWEEGPNSFQPSDPV
LTMAVDSGLKDDLVFGDPNAPRFVLWEGKLRPVPSKPADLPFFDLMSIPGKLRAGFGALGI
RPPPPGREESVEEFVRRNLGAEVFERLIEPFCSGVYAGDPSKLSMKAAFGKVWRLEEAGG
SIIGGTIKTIQERGKNPKPPRDPRLPTPKGQTVASFRKGLAMLPNAITSSLGSKVKLSWKLTSI
TKSDGMGYVLVYETPEGVVSVQAKSVIMTIPSYVASDILRPLSSDAADALSRFPYPPVAAVTI
SYPKEAIRKECLIDGELQGFGQLHPRSQGVETLGTIYSSSLFPNRAPAGRVLLLNYIGGATNT
GIVSKSASELVEAVDRDLRKMLINPSAVDPLVLGVRVWPQAIPQFLVGHLDLLEAAKSSLDR
GGYDGLFLGGNYVAGVALGRCVEGAYESASQISDFLTKYAYK*
Setaria italica PPO1 Y359Q (SEQ ID NO: 94)
MVAAAMATAPSAGVPPLRGTRGPARFRIRGVSVRCAAVAGGAAEAPASAGARVSADCVVV
GGGISGLCTAQALATKHGVGDVLVTEARARPGGNITTVERPDEGYLWEEGPNSFQPSDPV
LTMAVDSGLKDDLVFGDPNAPRFVLWEGKLRPVPSKPADLPFFDLMSIPGKLRAGFGALGI
RPPPPGREESVEEFVRRNLGAEVFERLIEPFCSGVYAGDPSKLSMKAAFGKVWRLEEAGG
SIIGGTIKTIQERGKNPKPPRDPRLPTPKGQTVASFRKGLAMLPNAITSSLGSKVKLSWKLTSI
TKSDGMGYVLVYETPEGVVSVQAKSVIMTIPSYVASDILRPLSSDAADALSRFQYPPVAAVTI
SYPKEAIRKECLIDGELQGFGQLHPRSQGVETLGTIYSSSLFPNRAPAGRVLLLNYIGGATNT
GIVSKSASELVEAVDRDLRKMLINPSAVDPLVLGVRVWPQAIPQFLVGHLDLLEAAKSSLDR
GGYDGLFLGGNYVAGVALGRCVEGAYESASQISDFLTKYAYK*
Setaria italica PPO1 Y359R (SEQ ID NO: 95)
MVAAAMATAPSAGVPPLRGTRGPARFRIRGVSVRCAAVAGGAAEAPASAGARVSADCVVV
GGGISGLCTAQALATKHGVGDVLVTEARARPGGNITTVERPDEGYLWEEGPNSFQPSDPV
LTMAVDSGLKDDLVFGDPNAPRFVLWEGKLRPVPSKPADLPFFDLMSIPGKLRAGFGALGI
RPPPPGREESVEEFVRRNLGAEVFERLIEPFCSGVYAGDPSKLSMKAAFGKVWRLEEAGG
SIIGGTIKTIQERGKNPKPPRDPRLPTPKGQTVASFRKGLAMLPNAITSSLGSKVKLSWKLTSI
TKSDGMGYVLVYETPEGVVSVQAKSVIMTIPSYVASDILRPLSSDAADALSRFRYPPVAAVTI
SYPKEAIRKECLIDGELQGFGQLHPRSQGVETLGTIYSSSLFPNRAPAGRVLLLNYIGGATNT
GIVSKSASELVEAVDRDLRKMLINPSAVDPLVLGVRVWPQAIPQFLVGHLDLLEAAKSSLDR
GGYDGLFLGGNYVAGVALGRCVEGAYESASQISDFLTKYAYK*
Setaria italica PPO1 Y359T (SEQ ID NO: 96)
MVAAAMATAPSAGVPPLRGTRGPARFRIRGVSVRCAAVAGGAAEAPASAGARVSADCVVV
GGGISGLCTAQALATKHGVGDVLVTEARARPGGNITTVERPDEGYLWEEGPNSFQPSDPV
LTMAVDSGLKDDLVFGDPNAPRFVLWEGKLRPVPSKPADLPFFDLMSIPGKLRAGFGALGI
RPPPPGREESVEEFVRRNLGAEVFERLIEPFCSGVYAGDPSKLSMKAAFGKVWRLEEAGG
SIIGGTIKTIQERGKNPKPPRDPRLPTPKGQTVASFRKGLAMLPNAITSSLGSKVKLSWKLTSI
TKSDGMGYVLVYETPEGVVSVQAKSVIMTIPSYVASDILRPLSSDAADALSRFTYPPVAAVTI
SYPKEAIRKECLIDGELQGFGQLHPRSQGVETLGTIYSSSLFPNRAPAGRVLLLNYIGGATNT
GIVSKSASELVEAVDRDLRKMLINPSAVDPLVLGVRVWPQAIPQFLVGHLDLLEAAKSSLDR
GGYDGLFLGGNYVAGVALGRCVEGAYESASQISDFLTKYAYK*
Setaria italica PPO1 Y360F (SEQ ID NO: 97)
MVAAAMATAPSAGVPPLRGTRGPARFRIRGVSVRCAAVAGGAAEAPASAGARVSADCVVV
GGGISGLCTAQALATKHGVGDVLVTEARARPGGNITTVERPDEGYLWEEGPNSFQPSDPV
LTMAVDSGLKDDLVFGDPNAPRFVLWEGKLRPVPSKPADLPFFDLMSIPGKLRAGFGALGI
RPPPPGREESVEEFVRRNLGAEVFERLIEPFCSGVYAGDPSKLSMKAAFGKVWRLEEAGG
SIIGGTIKTIQERGKNPKPPRDPRLPTPKGQTVASFRKGLAMLPNAITSSLGSKVKLSWKLTSI
TKSDGMGYVLVYETPEGVVSVQAKSVIMTIPSYVASDILRPLSSDAADALSRFYFPPVAAVTI
SYPKEAIRKECLIDGELQGFGQLHPRSQGVETLGTIYSSSLFPNRAPAGRVLLLNYIGGATNT
GIVSKSASELVEAVDRDLRKMLINPSAVDPLVLGVRVWPQAIPQFLVGHLDLLEAAKSSLDR
GGYDGLFLGGNYVAGVALGRCVEGAYESASQISDFLTKYAYK*
Setaria italica PPO1 V363L (SEQ ID NO: 98)
MVAAAMATAPSAGVPPLRGTRGPARFRIRGVSVRCAAVAGGAAEAPASAGARVSADCVVV
GGGISGLCTAQALATKHGVGDVLVTEARARPGGNITTVERPDEGYLWEEGPNSFQPSDPV
LTMAVDSGLKDDLVFGDPNAPRFVLWEGKLRPVPSKPADLPFFDLMSIPGKLRAGFGALGI
RPPPPGREESVEEFVRRNLGAEVFERLIEPFCSGVYAGDPSKLSMKAAFGKVWRLEEAGG
SIIGGTIKTIQERGKNPKPPRDPRLPTPKGQTVASFRKGLAMLPNAITSSLGSKVKLSWKLTSI
TKSDGMGYVLVYETPEGVVSVQAKSVIMTIPSYVASDILRPLSSDAADALSRFYYPPLAAVTI
SYPKEAIRKECLIDGELQGFGQLHPRSQGVETLGTIYSSSLFPNRAPAGRVLLLNYIGGATNT
GIVSKSASELVEAVDRDLRKMLINPSAVDPLVLGVRVWPQAIPQFLVGHLDLLEAAKSSLDR
GGYDGLFLGGNYVAGVALGRCVEGAYESASQISDFLTKYAYK*
Setaria italica PPO1 V363M (SEQ ID NO: 99)
MVAAAMATAPSAGVPPLRGTRGPARFRIRGVSVRCAAVAGGAAEAPASAGARVSADCVVV
GGGISGLCTAQALATKHGVGDVLVTEARARPGGNITTVERPDEGYLWEEGPNSFQPSDPV
LTMAVDSGLKDDLVFGDPNAPRFVLWEGKLRPVPSKPADLPFFDLMSIPGKLRAGFGALGI
RPPPPGREESVEEFVRRNLGAEVFERLIEPFCSGVYAGDPSKLSMKAAFGKVWRLEEAGG
SIIGGTIKTIQERGKNPKPPRDPRLPTPKGQTVASFRKGLAMLPNAITSSLGSKVKLSWKLTSI
TKSDGMGYVLVYETPEGVVSVQAKSVIMTIPSYVASDILRPLSSDAADALSRFYYPPMAAVTI
SYPKEAIRKECLIDGELQGFGQLHPRSQGVETLGTIYSSSLFPNRAPAGRVLLLNYIGGATNT
GIVSKSASELVEAVDRDLRKMLINPSAVDPLVLGVRVWPQAIPQFLVGHLDLLEAAKSSLDR
GGYDGLFLGGNYVAGVALGRCVEGAYESASQISDFLTKYAYK*
Setaria italica PPO1 G402A (SEQ ID NO: 100)
MVAAAMATAPSAGVPPLRGTRGPARFRIRGVSVRCAAVAGGAAEAPASAGARVSADCVVV
GGGISGLCTAQALATKHGVGDVLVTEARARPGGNITTVERPDEGYLWEEGPNSFQPSDPV
LTMAVDSGLKDDLVFGDPNAPRFVLWEGKLRPVPSKPADLPFFDLMSIPGKLRAGFGALGI
RPPPPGREESVEEFVRRNLGAEVFERLIEPFCSGVYAGDPSKLSMKAAFGKVWRLEEAGG
SIIGGTIKTIQERGKNPKPPRDPRLPTPKGQTVASFRKGLAMLPNAITSSLGSKVKLSWKLTSI
TKSDGMGYVLVYETPEGVVSVQAKSVIMTIPSYVASDILRPLSSDAADALSRFYYPPVAAVTI
SYPKEAIRKECLIDGELQGFGQLHPRSQGVETLATIYSSSLFPNRAPAGRVLLLNYIGGATNT
GIVSKSASELVEAVDRDLRKMLINPSAVDPLVLGVRVWPQAIPQFLVGHLDLLEAAKSSLDR
GGYDGLFLGGNYVAGVALGRCVEGAYESASQISDFLTKYAYK*
Setaria italica PPO1 P411G (SEQ ID NO: 101)
MVAAAMATAPSAGVPPLRGTRGPARFRIRGVSVRCAAVAGGAAEAPASAGARVSADCVVV
GGGISGLCTAQALATKHGVGDVLVTEARARPGGNITTVERPDEGYLWEEGPNSFQPSDPV
LTMAVDSGLKDDLVFGDPNAPRFVLWEGKLRPVPSKPADLPFFDLMSIPGKLRAGFGALGI
RPPPPGREESVEEFVRRNLGAEVFERLIEPFCSGVYAGDPSKLSMKAAFGKVWRLEEAGG
SIIGGTIKTIQERGKNPKPPRDPRLPTPKGQTVASFRKGLAMLPNAITSSLGSKVKLSWKLTSI
TKSDGMGYVLVYETPEGVVSVQAKSVIMTIPSYVASDILRPLSSDAADALSRFYYPPVAAVTI
SYPKEAIRKECLIDGELQGFGQLHPRSQGVETLGTIYSSSLFGNRAPAGRVLLLNYIGGATN
TGIVSKSASELVEAVDRDLRKMLINPSAVDPLVLGVRVWPQAIPQFLVGHLDLLEAAKSSLD
RGGYDGLFLGGNYVAGVALGRCVEGAYESASQISDFLTKYAYK*
Setaria italica PPO1 A416M (SEQ ID NO: 102)
MVAAAMATAPSAGVPPLRGTRGPARFRIRGVSVRCAAVAGGAAEAPASAGARVSADCVVV
GGGISGLCTAQALATKHGVGDVLVTEARARPGGNITTVERPDEGYLWEEGPNSFQPSDPV
LTMAVDSGLKDDLVFGDPNAPRFVLWEGKLRPVPSKPADLPFFDLMSIPGKLRAGFGALGI
RPPPPGREESVEEFVRRNLGAEVFERLIEPFCSGVYAGDPSKLSMKAAFGKVWRLEEAGG
SIIGGTIKTIQERGKNPKPPRDPRLPTPKGQTVASFRKGLAMLPNAITSSLGSKVKLSWKLTSI
TKSDGMGYVLVYETPEGVVSVQAKSVIMTIPSYVASDILRPLSSDAADALSRFYYPPVAAVTI
SYPKEAIRKECLIDGELQGFGQLHPRSQGVETLGTIYSSSLFPNRAPMGRVLLLNYIGGATN
TGIVSKSASELVEAVDRDLRKMLINPSAVDPLVLGVRVWPQAIPQFLVGHLDLLEAAKSSLD
RGGYDGLFLGGNYVAGVALGRCVEGAYESASQISDFLTKYAYK*
Setaria italica PPO1 L422V (SEQ ID NO: 103)
MVAAAMATAPSAGVPPLRGTRGPARFRIRGVSVRCAAVAGGAAEAPASAGARVSADCVVV
GGGISGLCTAQALATKHGVGDVLVTEARARPGGNITTVERPDEGYLWEEGPNSFQPSDPV
LTMAVDSGLKDDLVFGDPNAPRFVLWEGKLRPVPSKPADLPFFDLMSIPGKLRAGFGALGI
RPPPPGREESVEEFVRRNLGAEVFERLIEPFCSGVYAGDPSKLSMKAAFGKVWRLEEAGG
SIIGGTIKTIQERGKNPKPPRDPRLPTPKGQTVASFRKGLAMLPNAITSSLGSKVKLSWKLTSI
TKSDGMGYVLVYETPEGVVSVQAKSVIMTIPSYVASDILRPLSSDAADALSRFYYPPVAAVTI
SYPKEAIRKECLIDGELQGFGQLHPRSQGVETLGTIYSSSLFPNRAPAGRVLLVNYIGGATN
TGIVSKSASELVEAVDRDLRKMLINPSAVDPLVLGVRVWPQAIPQFLVGHLDLLEAAKSSLD
RGGYDGLFLGGNYVAGVALGRCVEGAYESASQISDFLTKYAYK*
Setaria italica PPO1 Y424C (SEQ ID NO: 104)
MVAAAMATAPSAGVPPLRGTRGPARFRIRGVSVRCAAVAGGAAEAPASAGARVSADCVVV
GGGISGLCTAQALATKHGVGDVLVTEARARPGGNITTVERPDEGYLWEEGPNSFQPSDPV
LTMAVDSGLKDDLVFGDPNAPRFVLWEGKLRPVPSKPADLPFFDLMSIPGKLRAGFGALGI
RPPPPGREESVEEFVRRNLGAEVFERLIEPFCSGVYAGDPSKLSMKAAFGKVWRLEEAGG
SIIGGTIKTIQERGKNPKPPRDPRLPTPKGQTVASFRKGLAMLPNAITSSLGSKVKLSWKLTSI
TKSDGMGYVLVYETPEGVVSVQAKSVIMTIPSYVASDILRPLSSDAADALSRFYYPPVAAVTI
SYPKEAIRKECLIDGELQGFGQLHPRSQGVETLGTIYSSSLFPNRAPAGRVLLLNCIGGATN
TGIVSKSASELVEAVDRDLRKMLINPSAVDPLVLGVRVWPQAIPQFLVGHLDLLEAAKSSLD
RGGYDGLFLGGNYVAGVALGRCVEGAYESASQISDFLTKYAYK*
Setaria italica PPO1 Y424L (SEQ ID NO: 105)
MVAAAMATAPSAGVPPLRGTRGPARFRIRGVSVRCAAVAGGAAEAPASAGARVSADCVVV
GGGISGLCTAQALATKHGVGDVLVTEARARPGGNITTVERPDEGYLWEEGPNSFQPSDPV
LTMAVDSGLKDDLVFGDPNAPRFVLWEGKLRPVPSKPADLPFFDLMSIPGKLRAGFGALGI
RPPPPGREESVEEFVRRNLGAEVFERLIEPFCSGVYAGDPSKLSMKAAFGKVWRLEEAGG
SIIGGTIKTIQERGKNPKPPRDPRLPTPKGQTVASFRKGLAMLPNAITSSLGSKVKLSWKLTSI
TKSDGMGYVLVYETPEGVVSVQAKSVIMTIPSYVASDILRPLSSDAADALSRFYYPPVAAVTI
SYPKEAIRKECLIDGELQGFGQLHPRSQGVETLGTIYSSSLFPNRAPAGRVLLLNLIGGATNT
GIVSKSASELVEAVDRDLRKMLINPSAVDPLVLGVRVWPQAIPQFLVGHLDLLEAAKSSLDR
GGYDGLFLGGNYVAGVALGRCVEGAYESASQISDFLTKYAYK*
Setaria italica PPO1 Y424M (SEQ ID NO: 106)
MVAAAMATAPSAGVPPLRGTRGPARFRIRGVSVRCAAVAGGAAEAPASAGARVSADCVVV
GGGISGLCTAQALATKHGVGDVLVTEARARPGGNITTVERPDEGYLWEEGPNSFQPSDPV
LTMAVDSGLKDDLVFGDPNAPRFVLWEGKLRPVPSKPADLPFFDLMSIPGKLRAGFGALGI
RPPPPGREESVEEFVRRNLGAEVFERLIEPFCSGVYAGDPSKLSMKAAFGKVWRLEEAGG
SIIGGTIKTIQERGKNPKPPRDPRLPTPKGQTVASFRKGLAMLPNAITSSLGSKVKLSWKLTSI
TKSDGMGYVLVYETPEGVVSVQAKSVIMTIPSYVASDILRPLSSDAADALSRFYYPPVAAVTI
SYPKEAIRKECLIDGELQGFGQLHPRSQGVETLGTIYSSSLFPNRAPAGRVLLLNMIGGATN
TGIVSKSASELVEAVDRDLRKMLINPSAVDPLVLGVRVWPQAIPQFLVGHLDLLEAAKSSLD
RGGYDGLFLGGNYVAGVALGRCVEGAYESASQISDFLTKYAYK*
Setaria italica PPO1 Y424T (SEQ ID NO: 107)
MVAAAMATAPSAGVPPLRGTRGPARFRIRGVSVRCAAVAGGAAEAPASAGARVSADCVVV
GGGISGLCTAQALATKHGVGDVLVTEARARPGGNITTVERPDEGYLWEEGPNSFQPSDPV
LTMAVDSGLKDDLVFGDPNAPRFVLWEGKLRPVPSKPADLPFFDLMSIPGKLRAGFGALGI
RPPPPGREESVEEFVRRNLGAEVFERLIEPFCSGVYAGDPSKLSMKAAFGKVWRLEEAGG
SIIGGTIKTIQERGKNPKPPRDPRLPTPKGQTVASFRKGLAMLPNAITSSLGSKVKLSWKLTSI
TKSDGMGYVLVYETPEGVVSVQAKSVIMTIPSYVASDILRPLSSDAADALSRFYYPPVAAVTI
SYPKEAIRKECLIDGELQGFGQLHPRSQGVETLGTIYSSSLFPNRAPAGRVLLLNTIGGATNT
GIVSKSASELVEAVDRDLRKMLINPSAVDPLVLGVRVWPQAIPQFLVGHLDLLEAAKSSLDR
GGYDGLFLGGNYVAGVALGRCVEGAYESASQISDFLTKYAYK*
Setaria italica PPO1 Y424V (SEQ ID NO: 108)
MVAAAMATAPSAGVPPLRGTRGPARFRIRGVSVRCAAVAGGAAEAPASAGARVSADCVVV
GGGISGLCTAQALATKHGVGDVLVTEARARPGGNITTVERPDEGYLWEEGPNSFQPSDPV
LTMAVDSGLKDDLVFGDPNAPRFVLWEGKLRPVPSKPADLPFFDLMSIPGKLRAGFGALGI
RPPPPGREESVEEFVRRNLGAEVFERLIEPFCSGVYAGDPSKLSMKAAFGKVWRLEEAGG
SIIGGTIKTIQERGKNPKPPRDPRLPTPKGQTVASFRKGLAMLPNAITSSLGSKVKLSWKLTSI
TKSDGMGYVLVYETPEGVVSVQAKSVIMTIPSYVASDILRPLSSDAADALSRFYYPPVAAVTI
SYPKEAIRKECLIDGELQGFGQLHPRSQGVETLGTIYSSSLFPNRAPAGRVLLLNVIGGATNT
GIVSKSASELVEAVDRDLRKMLINPSAVDPLVLGVRVWPQAIPQFLVGHLDLLEAAKSSLDR
GGYDGLFLGGNYVAGVALGRCVEGAYESASQISDFLTKYAYK*
Setaria italica PPO1 T429R (SEQ ID NO: 109)
MVAAAMATAPSAGVPPLRGTRGPARFRIRGVSVRCAAVAGGAAEAPASAGARVSADCVVV
GGGISGLCTAQALATKHGVGDVLVTEARARPGGNITTVERPDEGYLWEEGPNSFQPSDPV
LTMAVDSGLKDDLVFGDPNAPRFVLWEGKLRPVPSKPADLPFFDLMSIPGKLRAGFGALGI
RPPPPGREESVEEFVRRNLGAEVFERLIEPFCSGVYAGDPSKLSMKAAFGKVWRLEEAGG
SIIGGTIKTIQERGKNPKPPRDPRLPTPKGQTVASFRKGLAMLPNAITSSLGSKVKLSWKLTSI
TKSDGMGYVLVYETPEGVVSVQAKSVIMTIPSYVASDILRPLSSDAADALSRFYYPPVAAVTI
SYPKEAIRKECLIDGELQGFGQLHPRSQGVETLGTIYSSSLFPNRAPAGRVLLLNYIGGARN
TGIVSKSASELVEAVDRDLRKMLINPSAVDPLVLGVRVWPQAIPQFLVGHLDLLEAAKSSLD
RGGYDGLFLGGNYVAGVALGRCVEGAYESASQISDFLTKYAYK*
Setaria italica PPO1 V445C (SEQ ID NO: 110)
MVAAAMATAPSAGVPPLRGTRGPARFRIRGVSVRCAAVAGGAAEAPASAGARVSADCVVV
GGGISGLCTAQALATKHGVGDVLVTEARARPGGNITTVERPDEGYLWEEGPNSFQPSDPV
LTMAVDSGLKDDLVFGDPNAPRFVLWEGKLRPVPSKPADLPFFDLMSIPGKLRAGFGALGI
RPPPPGREESVEEFVRRNLGAEVFERLIEPFCSGVYAGDPSKLSMKAAFGKVWRLEEAGG
SIIGGTIKTIQERGKNPKPPRDPRLPTPKGQTVASFRKGLAMLPNAITSSLGSKVKLSWKLTSI
TKSDGMGYVLVYETPEGVVSVQAKSVIMTIPSYVASDILRPLSSDAADALSRFYYPPVAAVTI
SYPKEAIRKECLIDGELQGFGQLHPRSQGVETLGTIYSSSLFPNRAPAGRVLLLNYIGGATNT
GIVSKSASELVEACDRDLRKMLINPSAVDPLVLGVRVWPQAIPQFLVGHLDLLEAAKSSLDR
GGYDGLFLGGNYVAGVALGRCVEGAYESASQISDFLTKYAYK*
Setaria italica PPO1 V445I (SEQ ID NO: 111)
MVAAAMATAPSAGVPPLRGTRGPARFRIRGVSVRCAAVAGGAAEAPASAGARVSADCVVV
GGGISGLCTAQALATKHGVGDVLVTEARARPGGNITTVERPDEGYLWEEGPNSFQPSDPV
LTMAVDSGLKDDLVFGDPNAPRFVLWEGKLRPVPSKPADLPFFDLMSIPGKLRAGFGALGI
RPPPPGREESVEEFVRRNLGAEVFERLIEPFCSGVYAGDPSKLSMKAAFGKVWRLEEAGG
SIIGGTIKTIQERGKNPKPPRDPRLPTPKGQTVASFRKGLAMLPNAITSSLGSKVKLSWKLTSI
TKSDGMGYVLVYETPEGVVSVQAKSVIMTIPSYVASDILRPLSSDAADALSRFYYPPVAAVTI
SYPKEAIRKECLIDGELQGFGQLHPRSQGVETLGTIYSSSLFPNRAPAGRVLLLNYIGGATNT
GIVSKSASELVEAIDRDLRKMLINPSAVDPLVLGVRVWPQAIPQFLVGHLDLLEAAKSSLDRG
GYDGLFLGGNYVAGVALGRCVEGAYESASQISDFLTKYAYK*
Setaria italica PPO1 D446H (SEQ ID NO: 112)
MVAAAMATAPSAGVPPLRGTRGPARFRIRGVSVRCAAVAGGAAEAPASAGARVSADCVVV
GGGISGLCTAQALATKHGVGDVLVTEARARPGGNITTVERPDEGYLWEEGPNSFQPSDPV
LTMAVDSGLKDDLVFGDPNAPRFVLWEGKLRPVPSKPADLPFFDLMSIPGKLRAGFGALGI
RPPPPGREESVEEFVRRNLGAEVFERLIEPFCSGVYAGDPSKLSMKAAFGKVWRLEEAGG
SIIGGTIKTIQERGKNPKPPRDPRLPTPKGQTVASFRKGLAMLPNAITSSLGSKVKLSWKLTSI
TKSDGMGYVLVYETPEGVVSVQAKSVIMTIPSYVASDILRPLSSDAADALSRFYYPPVAAVTI
SYPKEAIRKECLIDGELQGFGQLHPRSQGVETLGTIYSSSLFPNRAPAGRVLLLNYIGGATNT
GIVSKSASELVEAVHRDLRKMLINPSAVDPLVLGVRVWPQAIPQFLVGHLDLLEAAKSSLDR
GGYDGLFLGGNYVAGVALGRCVEGAYESASQISDFLTKYAYK*
Setaria italica PPO1 D446L (SEQ ID NO: 113)
MVAAAMATAPSAGVPPLRGTRGPARFRIRGVSVRCAAVAGGAAEAPASAGARVSADCVVV
GGGISGLCTAQALATKHGVGDVLVTEARARPGGNITTVERPDEGYLWEEGPNSFQPSDPV
LTMAVDSGLKDDLVFGDPNAPRFVLWEGKLRPVPSKPADLPFFDLMSIPGKLRAGFGALGI
RPPPPGREESVEEFVRRNLGAEVFERLIEPFCSGVYAGDPSKLSMKAAFGKVWRLEEAGG
SIIGGTIKTIQERGKNPKPPRDPRLPTPKGQTVASFRKGLAMLPNAITSSLGSKVKLSWKLTSI
TKSDGMGYVLVYETPEGVVSVQAKSVIMTIPSYVASDILRPLSSDAADALSRFYYPPVAAVTI
SYPKEAIRKECLIDGELQGFGQLHPRSQGVETLGTIYSSSLFPNRAPAGRVLLLNYIGGATNT
GIVSKSASELVEAVLRDLRKMLINPSAVDPLVLGVRVWPQAIPQFLVGHLDLLEAAKSSLDR
GGYDGLFLGGNYVAGVALGRCVEGAYESASQISDFLTKYAYK*
Setaria italica PPO1 D446N (SEQ ID NO: 114)
MVAAAMATAPSAGVPPLRGTRGPARFRIRGVSVRCAAVAGGAAEAPASAGARVSADCVVV
GGGISGLCTAQALATKHGVGDVLVTEARARPGGNITTVERPDEGYLWEEGPNSFQPSDPV
LTMAVDSGLKDDLVFGDPNAPRFVLWEGKLRPVPSKPADLPFFDLMSIPGKLRAGFGALGI
RPPPPGREESVEEFVRRNLGAEVFERLIEPFCSGVYAGDPSKLSMKAAFGKVWRLEEAGG
SIIGGTIKTIQERGKNPKPPRDPRLPTPKGQTVASFRKGLAMLPNAITSSLGSKVKLSWKLTSI
TKSDGMGYVLVYETPEGVVSVQAKSVIMTIPSYVASDILRPLSSDAADALSRFYYPPVAAVTI
SYPKEAIRKECLIDGELQGFGQLHPRSQGVETLGTIYSSSLFPNRAPAGRVLLLNYIGGATNT
GIVSKSASELVEAVNRDLRKMLINPSAVDPLVLGVRVWPQAIPQFLVGHLDLLEAAKSSLDR
GGYDGLFLGGNYVAGVALGRCVEGAYESASQISDFLTKYAYK*
Setaria italica PPO1 R447K (SEQ ID NO: 115)
MVAAAMATAPSAGVPPLRGTRGPARFRIRGVSVRCAAVAGGAAEAPASAGARVSADCVVV
GGGISGLCTAQALATKHGVGDVLVTEARARPGGNITTVERPDEGYLWEEGPNSFQPSDPV
LTMAVDSGLKDDLVFGDPNAPRFVLWEGKLRPVPSKPADLPFFDLMSIPGKLRAGFGALGI
RPPPPGREESVEEFVRRNLGAEVFERLIEPFCSGVYAGDPSKLSMKAAFGKVWRLEEAGG
SIIGGTIKTIQERGKNPKPPRDPRLPTPKGQTVASFRKGLAMLPNAITSSLGSKVKLSWKLTSI
TKSDGMGYVLVYETPEGVVSVQAKSVIMTIPSYVASDILRPLSSDAADALSRFYYPPVAAVTI
SYPKEAIRKECLIDGELQGFGQLHPRSQGVETLGTIYSSSLFPNRAPAGRVLLLNYIGGATNT
GIVSKSASELVEAVDKDLRKMLINPSAVDPLVLGVRVWPQAIPQFLVGHLDLLEAAKSSLDR
GGYDGLFLGGNYVAGVALGRCVEGAYESASQISDFLTKYAYK*
Setaria italica PPO1 V459Q (SEQ ID NO: 116)
MVAAAMATAPSAGVPPLRGTRGPARFRIRGVSVRCAAVAGGAAEAPASAGARVSADCVVV
GGGISGLCTAQALATKHGVGDVLVTEARARPGGNITTVERPDEGYLWEEGPNSFQPSDPV
LTMAVDSGLKDDLVFGDPNAPRFVLWEGKLRPVPSKPADLPFFDLMSIPGKLRAGFGALGI
RPPPPGREESVEEFVRRNLGAEVFERLIEPFCSGVYAGDPSKLSMKAAFGKVWRLEEAGG
SIIGGTIKTIQERGKNPKPPRDPRLPTPKGQTVASFRKGLAMLPNAITSSLGSKVKLSWKLTSI
TKSDGMGYVLVYETPEGVVSVQAKSVIMTIPSYVASDILRPLSSDAADALSRFYYPPVAAVTI
SYPKEAIRKECLIDGELQGFGQLHPRSQGVETLGTIYSSSLFPNRAPAGRVLLLNYIGGATNT
GIVSKSASELVEAVDRDLRKMLINPSAQDPLVLGVRVWPQAIPQFLVGHLDLLEAAKSSLDR
GGYDGLFLGGNYVAGVALGRCVEGAYESASQISDFLTKYAYK*
Setaria italica PPO1 F476R (SEQ ID NO: 117)
MVAAAMATAPSAGVPPLRGTRGPARFRIRGVSVRCAAVAGGAAEAPASAGARVSADCVVV
GGGISGLCTAQALATKHGVGDVLVTEARARPGGNITTVERPDEGYLWEEGPNSFQPSDPV
LTMAVDSGLKDDLVFGDPNAPRFVLWEGKLRPVPSKPADLPFFDLMSIPGKLRAGFGALGI
RPPPPGREESVEEFVRRNLGAEVFERLIEPFCSGVYAGDPSKLSMKAAFGKVWRLEEAGG
SIIGGTIKTIQERGKNPKPPRDPRLPTPKGQTVASFRKGLAMLPNAITSSLGSKVKLSWKLTSI
TKSDGMGYVLVYETPEGVVSVQAKSVIMTIPSYVASDILRPLSSDAADALSRFYYPPVAAVTI
SYPKEAIRKECLIDGELQGFGQLHPRSQGVETLGTIYSSSLFPNRAPAGRVLLLNYIGGATNT
GIVSKSASELVEAVDRDLRKMLINPSAVDPLVLGVRVWPQAIPQRLVGHLDLLEAAKSSLDR
GGYDGLFLGGNYVAGVALGRCVEGAYESASQISDFLTKYAYK*
Setaria italica PPO1 L477M (SEQ ID NO: 118)
MVAAAMATAPSAGVPPLRGTRGPARFRIRGVSVRCAAVAGGAAEAPASAGARVSADCVVV
GGGISGLCTAQALATKHGVGDVLVTEARARPGGNITTVERPDEGYLWEEGPNSFQPSDPV
LTMAVDSGLKDDLVFGDPNAPRFVLWEGKLRPVPSKPADLPFFDLMSIPGKLRAGFGALGI
RPPPPGREESVEEFVRRNLGAEVFERLIEPFCSGVYAGDPSKLSMKAAFGKVWRLEEAGG
SIIGGTIKTIQERGKNPKPPRDPRLPTPKGQTVASFRKGLAMLPNAITSSLGSKVKLSWKLTSI
TKSDGMGYVLVYETPEGVVSVQAKSVIMTIPSYVASDILRPLSSDAADALSRFYYPPVAAVTI
SYPKEAIRKECLIDGELQGFGQLHPRSQGVETLGTIYSSSLFPNRAPAGRVLLLNYIGGATNT
GIVSKSASELVEAVDRDLRKMLINPSAVDPLVLGVRVWPQAIPQFMVGHLDLLEAAKSSLDR
GGYDGLFLGGNYVAGVALGRCVEGAYESASQISDFLTKYAYK*
Setaria italica PPO1 L477N (SEQ ID NO: 119)
MVAAAMATAPSAGVPPLRGTRGPARFRIRGVSVRCAAVAGGAAEAPASAGARVSADCVVV
GGGISGLCTAQALATKHGVGDVLVTEARARPGGNITTVERPDEGYLWEEGPNSFQPSDPV
LTMAVDSGLKDDLVFGDPNAPRFVLWEGKLRPVPSKPADLPFFDLMSIPGKLRAGFGALGI
RPPPPGREESVEEFVRRNLGAEVFERLIEPFCSGVYAGDPSKLSMKAAFGKVWRLEEAGG
SIIGGTIKTIQERGKNPKPPRDPRLPTPKGQTVASFRKGLAMLPNAITSSLGSKVKLSWKLTSI
TKSDGMGYVLVYETPEGVVSVQAKSVIMTIPSYVASDILRPLSSDAADALSRFYYPPVAAVTI
SYPKEAIRKECLIDGELQGFGQLHPRSQGVETLGTIYSSSLFPNRAPAGRVLLLNYIGGATNT
GIVSKSASELVEAVDRDLRKMLINPSAVDPLVLGVRVWPQAIPQFNVGHLDLLEAAKSSLDR
GGYDGLFLGGNYVAGVALGRCVEGAYESASQISDFLTKYAYK*
Setaria italica PPO1 G508N (SEQ ID NO: 120)
MVAAAMATAPSAGVPPLRGTRGPARFRIRGVSVRCAAVAGGAAEAPASAGARVSADCVVV
GGGISGLCTAQALATKHGVGDVLVTEARARPGGNITTVERPDEGYLWEEGPNSFQPSDPV
LTMAVDSGLKDDLVFGDPNAPRFVLWEGKLRPVPSKPADLPFFDLMSIPGKLRAGFGALGI
RPPPPGREESVEEFVRRNLGAEVFERLIEPFCSGVYAGDPSKLSMKAAFGKVWRLEEAGG
SIIGGTIKTIQERGKNPKPPRDPRLPTPKGQTVASFRKGLAMLPNAITSSLGSKVKLSWKLTSI
TKSDGMGYVLVYETPEGVVSVQAKSVIMTIPSYVASDILRPLSSDAADALSRFYYPPVAAVTI
SYPKEAIRKECLIDGELQGFGQLHPRSQGVETLGTIYSSSLFPNRAPAGRVLLLNYIGGATNT
GIVSKSASELVEAVDRDLRKMLINPSAVDPLVLGVRVWPQAIPQFLVGHLDLLEAAKSSLDR
GGYDGLFLGGNYVANVALGRCVEGAYESASQISDFLTKYAYK*
Setaria italica PPO1 V509L (SEQ ID NO: 121)
MVAAAMATAPSAGVPPLRGTRGPARFRIRGVSVRCAAVAGGAAEAPASAGARVSADCVVV
GGGISGLCTAQALATKHGVGDVLVTEARARPGGNITTVERPDEGYLWEEGPNSFQPSDPV
LTMAVDSGLKDDLVFGDPNAPRFVLWEGKLRPVPSKPADLPFFDLMSIPGKLRAGFGALGI
RPPPPGREESVEEFVRRNLGAEVFERLIEPFCSGVYAGDPSKLSMKAAFGKVWRLEEAGG
SIIGGTIKTIQERGKNPKPPRDPRLPTPKGQTVASFRKGLAMLPNAITSSLGSKVKLSWKLTSI
TKSDGMGYVLVYETPEGVVSVQAKSVIMTIPSYVASDILRPLSSDAADALSRFYYPPVAAVTI
SYPKEAIRKECLIDGELQGFGQLHPRSQGVETLGTIYSSSLFPNRAPAGRVLLLNYIGGATNT
GIVSKSASELVEAVDRDLRKMLINPSAVDPLVLGVRVWPQAIPQFLVGHLDLLEAAKSSLDR
GGYDGLFLGGNYVAGLALGRCVEGAYESASQISDFLTKYAYK*
Setaria italica PPO1 V509N (SEQ ID NO: 122)
MVAAAMATAPSAGVPPLRGTRGPARFRIRGVSVRCAAVAGGAAEAPASAGARVSADCVVV
GGGISGLCTAQALATKHGVGDVLVTEARARPGGNITTVERPDEGYLWEEGPNSFQPSDPV
LTMAVDSGLKDDLVFGDPNAPRFVLWEGKLRPVPSKPADLPFFDLMSIPGKLRAGFGALGI
RPPPPGREESVEEFVRRNLGAEVFERLIEPFCSGVYAGDPSKLSMKAAFGKVWRLEEAGG
SIIGGTIKTIQERGKNPKPPRDPRLPTPKGQTVASFRKGLAMLPNAITSSLGSKVKLSWKLTSI
TKSDGMGYVLVYETPEGVVSVQAKSVIMTIPSYVASDILRPLSSDAADALSRFYYPPVAAVTI
SYPKEAIRKECLIDGELQGFGQLHPRSQGVETLGTIYSSSLFPNRAPAGRVLLLNYIGGATNT
GIVSKSASELVEAVDRDLRKMLINPSAVDPLVLGVRVWPQAIPQFLVGHLDLLEAAKSSLDR
GGYDGLFLGGNYVAGNALGRCVEGAYESASQISDFLTKYAYK*
Setaria italica PPO1 C514Q (SEQ ID NO: 123)
MVAAAMATAPSAGVPPLRGTRGPARFRIRGVSVRCAAVAGGAAEAPASAGARVSADCVVV
GGGISGLCTAQALATKHGVGDVLVTEARARPGGNITTVERPDEGYLWEEGPNSFQPSDPV
LTMAVDSGLKDDLVFGDPNAPRFVLWEGKLRPVPSKPADLPFFDLMSIPGKLRAGFGALGI
RPPPPGREESVEEFVRRNLGAEVFERLIEPFCSGVYAGDPSKLSMKAAFGKVWRLEEAGG
SIIGGTIKTIQERGKNPKPPRDPRLPTPKGQTVASFRKGLAMLPNAITSSLGSKVKLSWKLTSI
TKSDGMGYVLVYETPEGVVSVQAKSVIMTIPSYVASDILRPLSSDAADALSRFYYPPVAAVTI
SYPKEAIRKECLIDGELQGFGQLHPRSQGVETLGTIYSSSLFPNRAPAGRVLLLNYIGGATNT
GIVSKSASELVEAVDRDLRKMLINPSAVDPLVLGVRVWPQAIPQFLVGHLDLLEAAKSSLDR
GGYDGLFLGGNYVAGVALGRQVEGAYESASQISDFLTKYAYK*
Arabidopsis thaliana PPO1 S305L Y426M (SEQ ID NO: 124)
MELSLLRPTTQSLLPSFSKPNLRLNVYKPLRLRCSVAGGPTVGSSKIEGGGGTTITTDCVIV
GGGISGLCIAQALATKHPDAAPNLIVTEAKDRVGGNIITREENGFLWEEGPNSFQPSDPMLT
MVVDSGLKDDLVLGDPTAPRFVLWNGKLRPVPSKLTDLPFFDLMSIGGKIRAGFGALGIRPS
PPGREESVEEFVRRNLGDEVFERLIEPFCSGVYAGDPSKLSMKAAFGKVWKLEQNGGSIIG
GTFKAIQERKNAPKAERDPRLPKPQGQTVGSFRKGLRMLPEAISARLGSKVKLSWKLLGITK
LESGGYNLTYETPDGLVSVQSKSVVMTVPSHVASGLLRPLSESAANALSKLYYPPVAAVSIS
YPKEAIRTECLIDGELKGFGQLHPRTQGVETLGTIYSSSLFPNRAPPGRILLLNMIGGSTNTGI
LSKSEGELVEAVDRDLRKMLIKPNSTDPLKLGVRVWPQAIPQFLVGHFDILDTAKSSLTSSG
YEGLFLGGNYVAGVALGRCVEGAYETAIEVNNFMSRYAYK
Arabidopsis thaliana PPO1 S305L Y426V (SEQ ID NO: 125)
MELSLLRPTTQSLLPSFSKPNLRLNVYKPLRLRCSVAGGPTVGSSKIEGGGGTTITTDCVIV
GGGISGLCIAQALATKHPDAAPNLIVTEAKDRVGGNIITREENGFLWEEGPNSFQPSDPMLT
MVVDSGLKDDLVLGDPTAPRFVLWNGKLRPVPSKLTDLPFFDLMSIGGKIRAGFGALGIRPS
PPGREESVEEFVRRNLGDEVFERLIEPFCSGVYAGDPSKLSMKAAFGKVWKLEQNGGSIIG
GTFKAIQERKNAPKAERDPRLPKPQGQTVGSFRKGLRMLPEAISARLGSKVKLSWKLLGITK
LESGGYNLTYETPDGLVSVQSKSVVMTVPSHVASGLLRPLSESAANALSKLYYPPVAAVSIS
YPKEAIRTECLIDGELKGFGQLHPRTQGVETLGTIYSSSLFPNRAPPGRILLLNVIGGSTNTGI
LSKSEGELVEAVDRDLRKMLIKPNSTDPLKLGVRVWPQAIPQFLVGHFDILDTAKSSLTSSG
YEGLFLGGNYVAGVALGRCVEGAYETAIEVNNFMSRYAYK*
Arabidopsis thaliana PPO1 V365L Y426V L479N (SEQ ID NO: 126)
MELSLLRPTTQSLLPSFSKPNLRLNVYKPLRLRCSVAGGPTVGSSKIEGGGGTTITTDCVIV
GGGISGLCIAQALATKHPDAAPNLIVTEAKDRVGGNIITREENGFLWEEGPNSFQPSDPMLT
MVVDSGLKDDLVLGDPTAPRFVLWNGKLRPVPSKLTDLPFFDLMSIGGKIRAGFGALGIRPS
PPGREESVEEFVRRNLGDEVFERLIEPFCSGVYAGDPSKLSMKAAFGKVWKLEQNGGSIIG
GTFKAIQERKNAPKAERDPRLPKPQGQTVGSFRKGLRMLPEAISARLGSKVKLSWKLSGIT
KLESGGYNLTYETPDGLVSVQSKSVVMTVPSHVASGLLRPLSESAANALSKLYYPPLAAVSI
SYPKEAIRTECLIDGELKGFGQLHPRTQGVETLGTIYSSSLFPNRAPPGRILLLNVIGGSTNT
GILSKSEGELVEAVDRDLRKMLIKPNSTDPLKLGVRVWPQAIPQFNVGHFDILDTAKSSLTSS
GYEGLFLGGNYVAGVALGRCVEGAYETAIEVNNFMSRYAYK*
Arabidopsis thaliana PPO1 S305L G404A Y426V T431R L479N (SEQ ID NO: 127)
MELSLLRPTTQSLLPSFSKPNLRLNVYKPLRLRCSVAGGPTVGSSKIEGGGGTTITTDCVIV
GGGISGLCIAQALATKHPDAAPNLIVTEAKDRVGGNIITREENGFLWEEGPNSFQPSDPMLT
MVVDSGLKDDLVLGDPTAPRFVLWNGKLRPVPSKLTDLPFFDLMSIGGKIRAGFGALGIRPS
PPGREESVEEFVRRNLGDEVFERLIEPFCSGVYAGDPSKLSMKAAFGKVWKLEQNGGSIIG
GTFKAIQERKNAPKAERDPRLPKPQGQTVGSFRKGLRMLPEAISARLGSKVKLSWKLLGITK
LESGGYNLTYETPDGLVSVQSKSVVMTVPSHVASGLLRPLSESAANALSKLYYPPVAAVSIS
YPKEAIRTECLIDGELKGFGQLHPRTQGVETLATIYSSSLFPNRAPPGRILLLNVIGGSRNTGI
LSKSEGELVEAVDRDLRKMLIKPNSTDPLKLGVRVWPQAIPQFNVGHFDILDTAKSSLTSSG
YEGLFLGGNYVAGVALGRCVEGAYETAIEVNNFMSRYAYK*
Arabidopsis thaliana PPO1 S305L V365M (SEQ ID NO: 128)
MELSLLRPTTQSLLPSFSKPNLRLNVYKPLRLRCSVAGGPTVGSSKIEGGGGTTITTDCVIV
GGGISGLCIAQALATKHPDAAPNLIVTEAKDRVGGNIITREENGFLWEEGPNSFQPSDPMLT
MVVDSGLKDDLVLGDPTAPRFVLWNGKLRPVPSKLTDLPFFDLMSIGGKIRAGFGALGIRPS
PPGREESVEEFVRRNLGDEVFERLIEPFCSGVYAGDPSKLSMKAAFGKVWKLEQNGGSIIG
GTFKAIQERKNAPKAERDPRLPKPQGQTVGSFRKGLRMLPEAISARLGSKVKLSWKLLGITK
LESGGYNLTYETPDGLVSVQSKSVVMTVPSHVASGLLRPLSESAANALSKLYYPPMAAVSI
SYPKEAIRTECLIDGELKGFGQLHPRTQGVETLGTIYSSSLFPNRAPPGRILLLNYIGGSTNT
GILSKSEGELVEAVDRDLRKMLIKPNSTDPLKLGVRVWPQAIPQFLVGHFDILDTAKSSLTSS
GYEGLFLGGNYVAGVALGRCVEGAYETAIEVNNFMSRYAYK*
Arabidopsis thaliana PPO1 S305L Y362F G404A (SEQ ID NO: 129)
MELSLLRPTTQSLLPSFSKPNLRLNVYKPLRLRCSVAGGPTVGSSKIEGGGGTTITTDCVIV
GGGISGLCIAQALATKHPDAAPNLIVTEAKDRVGGNIITREENGFLWEEGPNSFQPSDPMLT
MVVDSGLKDDLVLGDPTAPRFVLWNGKLRPVPSKLTDLPFFDLMSIGGKIRAGFGALGIRPS
PPGREESVEEFVRRNLGDEVFERLIEPFCSGVYAGDPSKLSMKAAFGKVWKLEQNGGSIIG
GTFKAIQERKNAPKAERDPRLPKPQGQTVGSFRKGLRMLPEAISARLGSKVKLSWKLLGITK
LESGGYNLTYETPDGLVSVQSKSVVMTVPSHVASGLLRPLSESAANALSKLYFPPVAAVSIS
YPKEAIRTECLIDGELKGFGQLHPRTQGVETLATIYSSSLFPNRAPPGRILLLNYIGGSTNTGI
LSKSEGELVEAVDRDLRKMLIKPNSTDPLKLGVRVWPQAIPQFLVGHFDILDTAKSSLTSSG
YEGLFLGGNYVAGVALGRCVEGAYETAIEVNNFMSRYAYK*
Arabidopsis thaliana PPO1 S305L Y361T V365L T431R L479M (SEQ ID NO: 130)
MELSLLRPTTQSLLPSFSKPNLRLNVYKPLRLRCSVAGGPTVGSSKIEGGGGTTITTDCVIV
GGGISGLCIAQALATKHPDAAPNLIVTEAKDRVGGNIITREENGFLWEEGPNSFQPSDPMLT
MVVDSGLKDDLVLGDPTAPRFVLWNGKLRPVPSKLTDLPFFDLMSIGGKIRAGFGALGIRPS
PPGREESVEEFVRRNLGDEVFERLIEPFCSGVYAGDPSKLSMKAAFGKVWKLEQNGGSIIG
GTFKAIQERKNAPKAERDPRLPKPQGQTVGSFRKGLRMLPEAISARLGSKVKLSWKLLGITK
LESGGYNLTYETPDGLVSVQSKSVVMTVPSHVASGLLRPLSESAANALSKLTYPPLAAVSIS
YPKEAIRTECLIDGELKGFGQLHPRTQGVETLGTIYSSSLFPNRAPPGRILLLNYIGGSRNTGI
LSKSEGELVEAVDRDLRKMLIKPNSTDPLKLGVRVWPQAIPQFMVGHFDILDTAKSSLTSSG
YEGLFLGGNYVAGVALGRCVEGAYETAIEVNNFMSRYAYK*
Arabidopsis thaliana PPO1 S305L Y426V L479M (SEQ ID NO: 131)
MELSLLRPTTQSLLPSFSKPNLRLNVYKPLRLRCSVAGGPTVGSSKIEGGGGTTITTDCVIV
GGGISGLCIAQALATKHPDAAPNLIVTEAKDRVGGNIITREENGFLWEEGPNSFQPSDPMLT
MVVDSGLKDDLVLGDPTAPRFVLWNGKLRPVPSKLTDLPFFDLMSIGGKIRAGFGALGIRPS
PPGREESVEEFVRRNLGDEVFERLIEPFCSGVYAGDPSKLSMKAAFGKVWKLEQNGGSIIG
GTFKAIQERKNAPKAERDPRLPKPQGQTVGSFRKGLRMLPEAISARLGSKVKLSWKLLGITK
LESGGYNLTYETPDGLVSVQSKSVVMTVPSHVASGLLRPLSESAANALSKLYYPPVAAVSIS
YPKEAIRTECLIDGELKGFGQLHPRTQGVETLGTIYSSSLFPNRAPPGRILLLNVIGGSTNTGI
LSKSEGELVEAVDRDLRKMLIKPNSTDPLKLGVRVWPQAIPQFMVGHFDILDTAKSSLTSSG
YEGLFLGGNYVAGVALGRCVEGAYETAIEVNNFMSRYAYK*
Arabidopsis thaliana PPO1 Y426M T461Q L479M (SEQ ID NO: 132)
MELSLLRPTTQSLLPSFSKPNLRLNVYKPLRLRCSVAGGPTVGSSKIEGGGGTTITTDCVIV
GGGISGLCIAQALATKHPDAAPNLIVTEAKDRVGGNIITREENGFLWEEGPNSFQPSDPMLT
MVVDSGLKDDLVLGDPTAPRFVLWNGKLRPVPSKLTDLPFFDLMSIGGKIRAGFGALGIRPS
PPGREESVEEFVRRNLGDEVFERLIEPFCSGVYAGDPSKLSMKAAFGKVWKLEQNGGSIIG
GTFKAIQERKNAPKAERDPRLPKPQGQTVGSFRKGLRMLPEAISARLGSKVKLSWKLSGIT
KLESGGYNLTYETPDGLVSVQSKSVVMTVPSHVASGLLRPLSESAANALSKLYYPPVAAVSI
SYPKEAIRTECLIDGELKGFGQLHPRTQGVETLGTIYSSSLFPNRAPPGRILLLNMIGGSTNT
GILSKSEGELVEAVDRDLRKMLIKPNSQDPLKLGVRVWPQAIPQFMVGHFDILDTAKSSLTS
SGYEGLFLGGNYVAGVALGRCVEGAYETAIEVNNFMSRYAYK*
Arabidopsis thaliana PPO1 S305L Y361D V365M (SEQ ID NO: 133)
MELSLLRPTTQSLLPSFSKPNLRLNVYKPLRLRCSVAGGPTVGSSKIEGGGGTTITTDCVIV
GGGISGLCIAQALATKHPDAAPNLIVTEAKDRVGGNIITREENGFLWEEGPNSFQPSDPMLT
MVVDSGLKDDLVLGDPTAPRFVLWNGKLRPVPSKLTDLPFFDLMSIGGKIRAGFGALGIRPS
PPGREESVEEFVRRNLGDEVFERLIEPFCSGVYAGDPSKLSMKAAFGKVWKLEQNGGSIIG
GTFKAIQERKNAPKAERDPRLPKPQGQTVGSFRKGLRMLPEAISARLGSKVKLSWKLLGITK
LESGGYNLTYETPDGLVSVQSKSVVMTVPSHVASGLLRPLSESAANALSKLDYPPMAAVSI
SYPKEAIRTECLIDGELKGFGQLHPRTQGVETLGTIYSSSLFPNRAPPGRILLLNYIGGSTNT
GILSKSEGELVEAVDRDLRKMLIKPNSTDPLKLGVRVWPQAIPQFLVGHFDILDTAKSSLTSS
GYEGLFLGGNYVAGVALGRCVEGAYETAIEVNNFMSRYAYK*
Arabidopsis thaliana PPO1 S305L Y361C Y362F Y426M L479M (SEQ ID NO: 134)
MELSLLRPTTQSLLPSFSKPNLRLNVYKPLRLRCSVAGGPTVGSSKIEGGGGTTITTDCVIV
GGGISGLCIAQALATKHPDAAPNLIVTEAKDRVGGNIITREENGFLWEEGPNSFQPSDPMLT
MVVDSGLKDDLVLGDPTAPRFVLWNGKLRPVPSKLTDLPFFDLMSIGGKIRAGFGALGIRPS
PPGREESVEEFVRRNLGDEVFERLIEPFCSGVYAGDPSKLSMKAAFGKVWKLEQNGGSIIG
GTFKAIQERKNAPKAERDPRLPKPQGQTVGSFRKGLRMLPEAISARLGSKVKLSWKLLGITK
LESGGYNLTYETPDGLVSVQSKSVVMTVPSHVASGLLRPLSESAANALSKLCFPPVAAVSIS
YPKEAIRTECLIDGELKGFGQLHPRTQGVETLGTIYSSSLFPNRAPPGRILLLNMIGGSTNTGI
LSKSEGELVEAVDRDLRKMLIKPNSTDPLKLGVRVWPQAIPQFMVGHFDILDTAKSSLTSSG
YEGLFLGGNYVAGVALGRCVEGAYETAIEVNNFMSRYAYK*
Arabidopsis thaliana PPO1 Y361D V365L (SEQ ID NO: 135)
MELSLLRPTTQSLLPSFSKPNLRLNVYKPLRLRCSVAGGPTVGSSKIEGGGGTTITTDCVIV
GGGISGLCIAQALATKHPDAAPNLIVTEAKDRVGGNIITREENGFLWEEGPNSFQPSDPMLT
MVVDSGLKDDLVLGDPTAPRFVLWNGKLRPVPSKLTDLPFFDLMSIGGKIRAGFGALGIRPS
PPGREESVEEFVRRNLGDEVFERLIEPFCSGVYAGDPSKLSMKAAFGKVWKLEQNGGSIIG
GTFKAIQERKNAPKAERDPRLPKPQGQTVGSFRKGLRMLPEAISARLGSKVKLSWKLSGIT
KLESGGYNLTYETPDGLVSVQSKSVVMTVPSHVASGLLRPLSESAANALSKLDYPPLAAVSI
SYPKEAIRTECLIDGELKGFGQLHPRTQGVETLGTIYSSSLFPNRAPPGRILLLNYIGGSTNT
GILSKSEGELVEAVDRDLRKMLIKPNSTDPLKLGVRVWPQAIPQFLVGHFDILDTAKSSLTSS
GYEGLFLGGNYVAGVALGRCVEGAYETAIEVNNFMSRYAYK*
Arabidopsis thaliana PPO1 V365L G404A L479N (SEQ ID NO: 136)
MELSLLRPTTQSLLPSFSKPNLRLNVYKPLRLRCSVAGGPTVGSSKIEGGGGTTITTDCVIV
GGGISGLCIAQALATKHPDAAPNLIVTEAKDRVGGNIITREENGFLWEEGPNSFQPSDPMLT
MVVDSGLKDDLVLGDPTAPRFVLWNGKLRPVPSKLTDLPFFDLMSIGGKIRAGFGALGIRPS
PPGREESVEEFVRRNLGDEVFERLIEPFCSGVYAGDPSKLSMKAAFGKVWKLEQNGGSIIG
GTFKAIQERKNAPKAERDPRLPKPQGQTVGSFRKGLRMLPEAISARLGSKVKLSWKLSGIT
KLESGGYNLTYETPDGLVSVQSKSVVMTVPSHVASGLLRPLSESAANALSKLYYPPLAAVSI
SYPKEAIRTECLIDGELKGFGQLHPRTQGVETLATIYSSSLFPNRAPPGRILLLNYIGGSTNT
GILSKSEGELVEAVDRDLRKMLIKPNSTDPLKLGVRVWPQAIPQFNVGHFDILDTAKSSLTSS
GYEGLFLGGNYVAGVALGRCVEGAYETAIEVNNFMSRYAYK*
Arabidopsis thaliana PPO1 S305L G404A L479N (SEQ ID NO: 137)
MELSLLRPTTQSLLPSFSKPNLRLNVYKPLRLRCSVAGGPTVGSSKIEGGGGTTITTDCVIV
GGGISGLCIAQALATKHPDAAPNLIVTEAKDRVGGNIITREENGFLWEEGPNSFQPSDPMLT
MVVDSGLKDDLVLGDPTAPRFVLWNGKLRPVPSKLTDLPFFDLMSIGGKIRAGFGALGIRPS
PPGREESVEEFVRRNLGDEVFERLIEPFCSGVYAGDPSKLSMKAAFGKVWKLEQNGGSIIG
GTFKAIQERKNAPKAERDPRLPKPQGQTVGSFRKGLRMLPEAISARLGSKVKLSWKLLGITK
LESGGYNLTYETPDGLVSVQSKSVVMTVPSHVASGLLRPLSESAANALSKLYYPPVAAVSIS
YPKEAIRTECLIDGELKGFGQLHPRTQGVETLATIYSSSLFPNRAPPGRILLLNYIGGSTNTGI
LSKSEGELVEAVDRDLRKMLIKPNSTDPLKLGVRVWPQAIPQFNVGHFDILDTAKSSLTSSG
YEGLFLGGNYVAGVALGRCVEGAYETAIEVNNFMSRYAYK*
Setaria italica PPO1 T303L Y424M (SEQ ID NO: 138)
MVAAAMATAPSAGVPPLRGTRGPARFRIRGVSVRCAAVAGGAAEAPASAGARVSADCVVV
GGGISGLCTAQALATKHGVGDVLVTEARARPGGNITTVERPDEGYLWEEGPNSFQPSDPV
LTMAVDSGLKDDLVFGDPNAPRFVLWEGKLRPVPSKPADLPFFDLMSIPGKLRAGFGALGI
RPPPPGREESVEEFVRRNLGAEVFERLIEPFCSGVYAGDPSKLSMKAAFGKVWRLEEAGG
SIIGGTIKTIQERGKNPKPPRDPRLPTPKGQTVASFRKGLAMLPNAITSSLGSKVKLSWKLLSI
TKSDGMGYVLVYETPEGVVSVQAKSVIMTIPSYVASDILRPLSSDAADALSRFYYPPVAAVTI
SYPKEAIRKECLIDGELQGFGQLHPRSQGVETLGTIYSSSLFPNRAPAGRVLLLNMIGGATN
TGIVSKSASELVEAVDRDLRKMLINPSAVDPLVLGVRVWPQAIPQFLVGHLDLLEAAKSSLD
RGGYDGLFLGGNYVAGVALGRCVEGAYESASQISDFLTKYAYK*
Setaria italica PPO1 T303L Y424V (SEQ ID NO: 139)
MVAAAMATAPSAGVPPLRGTRGPARFRIRGVSVRCAAVAGGAAEAPASAGARVSADCVVV
GGGISGLCTAQALATKHGVGDVLVTEARARPGGNITTVERPDEGYLWEEGPNSFQPSDPV
LTMAVDSGLKDDLVFGDPNAPRFVLWEGKLRPVPSKPADLPFFDLMSIPGKLRAGFGALGI
RPPPPGREESVEEFVRRNLGAEVFERLIEPFCSGVYAGDPSKLSMKAAFGKVWRLEEAGG
SIIGGTIKTIQERGKNPKPPRDPRLPTPKGQTVASFRKGLAMLPNAITSSLGSKVKLSWKLLSI
TKSDGMGYVLVYETPEGVVSVQAKSVIMTIPSYVASDILRPLSSDAADALSRFYYPPVAAVTI
SYPKEAIRKECLIDGELQGFGQLHPRSQGVETLGTIYSSSLFPNRAPAGRVLLLNVIGGATNT
GIVSKSASELVEAVDRDLRKMLINPSAVDPLVLGVRVWPQAIPQFLVGHLDLLEAAKSSLDR
GGYDGLFLGGNYVAGVALGRCVEGAYESASQISDFLTKYAYK*
Setaria italica PPO1 V363L Y424V L477N (SEQ ID NO: 140)
MVAAAMATAPSAGVPPLRGTRGPARFRIRGVSVRCAAVAGGAAEAPASAGARVSADCVVV
GGGISGLCTAQALATKHGVGDVLVTEARARPGGNITTVERPDEGYLWEEGPNSFQPSDPV
LTMAVDSGLKDDLVFGDPNAPRFVLWEGKLRPVPSKPADLPFFDLMSIPGKLRAGFGALGI
RPPPPGREESVEEFVRRNLGAEVFERLIEPFCSGVYAGDPSKLSMKAAFGKVWRLEEAGG
SIIGGTIKTIQERGKNPKPPRDPRLPTPKGQTVASFRKGLAMLPNAITSSLGSKVKLSWKLTSI
TKSDGMGYVLVYETPEGVVSVQAKSVIMTIPSYVASDILRPLSSDAADALSRFYYPPLAAVTI
SYPKEAIRKECLIDGELQGFGQLHPRSQGVETLGTIYSSSLFPNRAPAGRVLLLNVIGGATNT
GIVSKSASELVEAVDRDLRKMLINPSAVDPLVLGVRVWPQAIPQFNVGHLDLLEAAKSSLDR
GGYDGLFLGGNYVAGVALGRCVEGAYESASQISDFLTKYAYK*
Setaria italica PPO1 T303L G402A Y424V T429R L477N (SEQ ID NO: 141)
MVAAAMATAPSAGVPPLRGTRGPARFRIRGVSVRCAAVAGGAAEAPASAGARVSADCVVV
GGGISGLCTAQALATKHGVGDVLVTEARARPGGNITTVERPDEGYLWEEGPNSFQPSDPV
LTMAVDSGLKDDLVFGDPNAPRFVLWEGKLRPVPSKPADLPFFDLMSIPGKLRAGFGALGI
RPPPPGREESVEEFVRRNLGAEVFERLIEPFCSGVYAGDPSKLSMKAAFGKVWRLEEAGG
SIIGGTIKTIQERGKNPKPPRDPRLPTPKGQTVASFRKGLAMLPNAITSSLGSKVKLSWKLLSI
TKSDGMGYVLVYETPEGVVSVQAKSVIMTIPSYVASDILRPLSSDAADALSRFYYPPVAAVTI
SYPKEAIRKECLIDGELQGFGQLHPRSQGVETLATIYSSSLFPNRAPAGRVLLLNVIGGARNT
GIVSKSASELVEAVDRDLRKMLINPSAVDPLVLGVRVWPQAIPQFNVGHLDLLEAAKSSLDR
GGYDGLFLGGNYVAGVALGRCVEGAYESASQISDFLTKYAYK*
Setaria italica PPO1 T303L V363M (SEQ ID NO: 142)
MVAAAMATAPSAGVPPLRGTRGPARFRIRGVSVRCAAVAGGAAEAPASAGARVSADCVVV
GGGISGLCTAQALATKHGVGDVLVTEARARPGGNITTVERPDEGYLWEEGPNSFQPSDPV
LTMAVDSGLKDDLVFGDPNAPRFVLWEGKLRPVPSKPADLPFFDLMSIPGKLRAGFGALGI
RPPPPGREESVEEFVRRNLGAEVFERLIEPFCSGVYAGDPSKLSMKAAFGKVWRLEEAGG
SIIGGTIKTIQERGKNPKPPRDPRLPTPKGQTVASFRKGLAMLPNAITSSLGSKVKLSWKLLSI
TKSDGMGYVLVYETPEGVVSVQAKSVIMTIPSYVASDILRPLSSDAADALSRFYYPPMAAVTI
SYPKEAIRKECLIDGELQGFGQLHPRSQGVETLGTIYSSSLFPNRAPAGRVLLLNYIGGATNT
GIVSKSASELVEAVDRDLRKMLINPSAVDPLVLGVRVWPQAIPQFLVGHLDLLEAAKSSLDR
GGYDGLFLGGNYVAGVALGRCVEGAYESASQISDFLTKYAYK*
Setaria italica PPO1 T303L Y360F G402A (SEQ ID NO: 143)
MVAAAMATAPSAGVPPLRGTRGPARFRIRGVSVRCAAVAGGAAEAPASAGARVSADCVVV
GGGISGLCTAQALATKHGVGDVLVTEARARPGGNITTVERPDEGYLWEEGPNSFQPSDPV
LTMAVDSGLKDDLVFGDPNAPRFVLWEGKLRPVPSKPADLPFFDLMSIPGKLRAGFGALGI
RPPPPGREESVEEFVRRNLGAEVFERLIEPFCSGVYAGDPSKLSMKAAFGKVWRLEEAGG
SIIGGTIKTIQERGKNPKPPRDPRLPTPKGQTVASFRKGLAMLPNAITSSLGSKVKLSWKLLSI
TKSDGMGYVLVYETPEGVVSVQAKSVIMTIPSYVASDILRPLSSDAADALSRFYFPPVAAVTI
SYPKEAIRKECLIDGELQGFGQLHPRSQGVETLATIYSSSLFPNRAPAGRVLLLNYIGGATNT
GIVSKSASELVEAVDRDLRKMLINPSAVDPLVLGVRVWPQAIPQFLVGHLDLLEAAKSSLDR
GGYDGLFLGGNYVAGVALGRCVEGAYESASQISDFLTKYAYK*
Setaria italica PPO1 T303L Y359T V363L T429R L477M (SEQ ID NO: 144)
MVAAAMATAPSAGVPPLRGTRGPARFRIRGVSVRCAAVAGGAAEAPASAGARVSADCVVV
GGGISGLCTAQALATKHGVGDVLVTEARARPGGNITTVERPDEGYLWEEGPNSFQPSDPV
LTMAVDSGLKDDLVFGDPNAPRFVLWEGKLRPVPSKPADLPFFDLMSIPGKLRAGFGALGI
RPPPPGREESVEEFVRRNLGAEVFERLIEPFCSGVYAGDPSKLSMKAAFGKVWRLEEAGG
SIIGGTIKTIQERGKNPKPPRDPRLPTPKGQTVASFRKGLAMLPNAITSSLGSKVKLSWKLLSI
TKSDGMGYVLVYETPEGVVSVQAKSVIMTIPSYVASDILRPLSSDAADALSRFTYPPLAAVTI
SYPKEAIRKECLIDGELQGFGQLHPRSQGVETLGTIYSSSLFPNRAPAGRVLLLNYIGGAAN
TGIVSKSASELVEAVDRDLRKMLINPSAVDPLVLGVRVWPQAIPQFMVGHLDLLEAAKSSLD
RGGYDGLFLGGNYVAGVALGRCVEGAYESASQISDFLTKYAYK*
Setaria italica PPO1 T303L Y424V L477M (SEQ ID NO: 145)
MVAAAMATAPSAGVPPLRGTRGPARFRIRGVSVRCAAVAGGAAEAPASAGARVSADCVVV
GGGISGLCTAQALATKHGVGDVLVTEARARPGGNITTVERPDEGYLWEEGPNSFQPSDPV
LTMAVDSGLKDDLVFGDPNAPRFVLWEGKLRPVPSKPADLPFFDLMSIPGKLRAGFGALGI
RPPPPGREESVEEFVRRNLGAEVFERLIEPFCSGVYAGDPSKLSMKAAFGKVWRLEEAGG
SIIGGTIKTIQERGKNPKPPRDPRLPTPKGQTVASFRKGLAMLPNAITSSLGSKVKLSWKLLSI
TKSDGMGYVLVYETPEGVVSVQAKSVIMTIPSYVASDILRPLSSDAADALSRFYYPPVAAVTI
SYPKEAIRKECLIDGELQGFGQLHPRSQGVETLGTIYSSSLFPNRAPAGRVLLLNVIGGATNT
GIVSKSASELVEAVDRDLRKMLINPSAVDPLVLGVRVWPQAIPQFMVGHLDLLEAAKSSLDR
GGYDGLFLGGNYVAGVALGRCVEGAYESASQISDFLTKYAYK*
Setaria italica PPO1 Y424M V459Q L477M (SEQ ID NO: 146)
MVAAAMATAPSAGVPPLRGTRGPARFRIRGVSVRCAAVAGGAAEAPASAGARVSADCVVV
GGGISGLCTAQALATKHGVGDVLVTEARARPGGNITTVERPDEGYLWEEGPNSFQPSDPV
LTMAVDSGLKDDLVFGDPNAPRFVLWEGKLRPVPSKPADLPFFDLMSIPGKLRAGFGALGI
RPPPPGREESVEEFVRRNLGAEVFERLIEPFCSGVYAGDPSKLSMKAAFGKVWRLEEAGG
SIIGGTIKTIQERGKNPKPPRDPRLPTPKGQTVASFRKGLAMLPNAITSSLGSKVKLSWKLTSI
TKSDGMGYVLVYETPEGVVSVQAKSVIMTIPSYVASDILRPLSSDAADALSRFYYPPVAAVTI
SYPKEAIRKECLIDGELQGFGQLHPRSQGVETLGTIYSSSLFPNRAPAGRVLLLNMIGGATN
TGIVSKSASELVEAVDRDLRKMLINPSAQDPLVLGVRVWPQAIPQFMVGHLDLLEAAKSSLD
RGGYDGLFLGGNYVAGVALGRCVEGAYESASQISDFLTKYAYK*
Setaria italica PPO1 T303L Y359D V363M (SEQ ID NO: 147)
MVAAAMATAPSAGVPPLRGTRGPARFRIRGVSVRCAAVAGGAAEAPASAGARVSADCVVV
GGGISGLCTAQALATKHGVGDVLVTEARARPGGNITTVERPDEGYLWEEGPNSFQPSDPV
LTMAVDSGLKDDLVFGDPNAPRFVLWEGKLRPVPSKPADLPFFDLMSIPGKLRAGFGALGI
RPPPPGREESVEEFVRRNLGAEVFERLIEPFCSGVYAGDPSKLSMKAAFGKVWRLEEAGG
SIIGGTIKTIQERGKNPKPPRDPRLPTPKGQTVASFRKGLAMLPNAITSSLGSKVKLSWKLLSI
TKSDGMGYVLVYETPEGVVSVQAKSVIMTIPSYVASDILRPLSSDAADALSRFDYPPMAAVT
ISYPKEAIRKECLIDGELQGFGQLHPRSQGVETLGTIYSSSLFPNRAPAGRVLLLNYIGGATN
TGIVSKSASELVEAVDRDLRKMLINPSAVDPLVLGVRVWPQAIPQFLVGHLDLLEAAKSSLD
RGGYDGLFLGGNYVAGVALGRCVEGAYESASQISDFLTKYAYK*
Setaria italica PPO1 T303L Y359C Y360F Y424M L477M (SEQ ID NO: 148)
MVAAAMATAPSAGVPPLRGTRGPARFRIRGVSVRCAAVAGGAAEAPASAGARVSADCVVV
GGGISGLCTAQALATKHGVGDVLVTEARARPGGNITTVERPDEGYLWEEGPNSFQPSDPV
LTMAVDSGLKDDLVFGDPNAPRFVLWEGKLRPVPSKPADLPFFDLMSIPGKLRAGFGALGI
RPPPPGREESVEEFVRRNLGAEVFERLIEPFCSGVYAGDPSKLSMKAAFGKVWRLEEAGG
SIIGGTIKTIQERGKNPKPPRDPRLPTPKGQTVASFRKGLAMLPNAITSSLGSKVKLSWKLLSI
TKSDGMGYVLVYETPEGVVSVQAKSVIMTIPSYVASDILRPLSSDAADALSRFCFPPVAAVTI
SYPKEAIRKECLIDGELQGFGQLHPRSQGVETLGTIYSSSLFPNRAPAGRVLLLNMIGGATN
TGIVSKSASELVEAVDRDLRKMLINPSAVDPLVLGVRVWPQAIPQFMVGHLDLLEAAKSSLD
RGGYDGLFLGGNYVAGVALGRCVEGAYESASQISDFLTKYAYK*
Setaria italica PPO1 Y359D V363L (SEQ ID NO: 149)
MVAAAMATAPSAGVPPLRGTRGPARFRIRGVSVRCAAVAGGAAEAPASAGARVSADCVVV
GGGISGLCTAQALATKHGVGDVLVTEARARPGGNITTVERPDEGYLWEEGPNSFQPSDPV
LTMAVDSGLKDDLVFGDPNAPRFVLWEGKLRPVPSKPADLPFFDLMSIPGKLRAGFGALGI
RPPPPGREESVEEFVRRNLGAEVFERLIEPFCSGVYAGDPSKLSMKAAFGKVWRLEEAGG
SIIGGTIKTIQERGKNPKPPRDPRLPTPKGQTVASFRKGLAMLPNAITSSLGSKVKLSWKLTSI
TKSDGMGYVLVYETPEGVVSVQAKSVIMTIPSYVASDILRPLSSDAADALSRFDYPPLAAVTI
SYPKEAIRKECLIDGELQGFGQLHPRSQGVETLGTIYSSSLFPNRAPAGRVLLLNYIGGATNT
GIVSKSASELVEAVDRDLRKMLINPSAVDPLVLGVRVWPQAIPQFLVGHLDLLEAAKSSLDR
GGYDGLFLGGNYVAGVALGRCVEGAYESASQISDFLTKYAYK*
Setaria italica PPO1 V363L G402A L477N (SEQ ID NO: 150)
MVAAAMATAPSAGVPPLRGTRGPARFRIRGVSVRCAAVAGGAAEAPASAGARVSADCVVV
GGGISGLCTAQALATKHGVGDVLVTEARARPGGNITTVERPDEGYLWEEGPNSFQPSDPV
LTMAVDSGLKDDLVFGDPNAPRFVLWEGKLRPVPSKPADLPFFDLMSIPGKLRAGFGALGI
RPPPPGREESVEEFVRRNLGAEVFERLIEPFCSGVYAGDPSKLSMKAAFGKVWRLEEAGG
SIIGGTIKTIQERGKNPKPPRDPRLPTPKGQTVASFRKGLAMLPNAITSSLGSKVKLSWKLTSI
TKSDGMGYVLVYETPEGVVSVQAKSVIMTIPSYVASDILRPLSSDAADALSRFYYPPLAAVTI
SYPKEAIRKECLIDGELQGFGQLHPRSQGVETLATIYSSSLFPNRAPAGRVLLLNYIGGATNT
GIVSKSASELVEAVDRDLRKMLINPSAVDPLVLGVRVWPQAIPQFNVGHLDLLEAAKSSLDR
GGYDGLFLGGNYVAGVALGRCVEGAYESASQISDFLTKYAYK*
Setaria italica PPO1 T303L G402A L477N (SEQ ID NO: 151)
MVAAAMATAPSAGVPPLRGTRGPARFRIRGVSVRCAAVAGGAAEAPASAGARVSADCVVV
GGGISGLCTAQALATKHGVGDVLVTEARARPGGNITTVERPDEGYLWEEGPNSFQPSDPV
LTMAVDSGLKDDLVFGDPNAPRFVLWEGKLRPVPSKPADLPFFDLMSIPGKLRAGFGALGI
RPPPPGREESVEEFVRRNLGAEVFERLIEPFCSGVYAGDPSKLSMKAAFGKVWRLEEAGG
SIIGGTIKTIQERGKNPKPPRDPRLPTPKGQTVASFRKGLAMLPNAITSSLGSKVKLSWKLLSI
TKSDGMGYVLVYETPEGVVSVQAKSVIMTIPSYVASDILRPLSSDAADALSRFYYPPVAAVTI
SYPKEAIRKECLIDGELQGFGQLHPRSQGVETLATIYSSSLFPNRAPAGRVLLLNYIGGATNT
GIVSKSASELVEAVDRDLRKMLINPSAVDPLVLGVRVWPQAIPQFNVGHLDLLEAAKSSLDR
GGYDGLFLGGNYVAGVALGRCVEGAYESASQISDFLTKYAYK*
pBin TMV AtPPO1 S305L Y426V (SEQ ID NO: 152)
AGCTTGCATGCCTGCAGGTCAACATGGTGGAGCACGACACACTTGTCTACTCCAAAAAT
ATCAAAGATACAGTCTCAGAAGACCAAAGGGCAATTGAGACTTTTCAACAAAGGGTAAT
ATCCGGAAACCTCCTCGGATTCCATTGCCCAGCTATCTGTCACTTTATTGTGAAGATAG
TGGAAAAGGAAGGTGGCTCCTACAAATGCCATCATTGCGATAAAGGAAAGGCCATCGT
TGAAGATGCCTCTGCCGACAGTGGTCCCAAAGATGGACCCCCACCCACGAGGAGCATC
GTGGAAAAAGAAGACGTTCCAACCACGTCTTCAAAGCAAGTGGATTGATGTGATAACAT
GGTGGAGCACGACACACTTGTCTACTCCAAAAATATCAAAGATACAGTCTCAGAAGACC
AAAGGGCAATTGAGACTTTTCAACAAAGGGTAATATCCGGAAACCTCCTCGGATTCCAT
TGCCCAGCTATCTGTCACTTTATTGTGAAGATAGTGGAAAAGGAAGGTGGCTCTTACAA
ATGCCATCATTGCGATAAAGGAAAGGCCATCGTTGAAGATGCCTCTGCCGACAGTGGT
CCCAAAGATGGACCCCCACCCACGAGGAGCATCGTGGAAAAAGAAGACGTTCCAACCA
CGTCTTCAAAGCAAGTGGATTGATGTGATATCTCCACTGACGTAAGGGATGACGCACAA
TCCCACTATCCTTCGCAAGACCCTTCCTCTATATAAGGAAGTTCATTTCATTTGGAGAGG
ACCTCGAGTATTTTTACAACAATTACCAACAACAACAAACAACAAACAACATTACAATTA
CTATTTACAATTACACATATGGAGTTATCTCTTCTCCGTCCGACGACTCAATCGCTTCTT
CCGTCGTTTTCGAAGCCCAATCTCCGATTAAATGTTTATAAGCCTCTTAGACTCCGTTGT
TCAGTGGCCGGTGGACCAACCGTCGGATCTTCAAAAATCGAAGGCGGAGGAGGCACC
ACCATCACGACGGATTGTGTGATTGTCGGCGGAGGTATTAGTGGTCTTTGCATCGCTCA
GGCGCTTGCTACTAAGCATCCTGATGCTGCTCCGAATTTAATTGTGACCGAGGCTAAGG
ATCGTGTTGGAGGCAACATTATCACTCGTGAAGAGAATGGTTTTCTCTGGGAAGAAGGT
CCCAATAGTTTTCAACCGTCTGATCCTATGCTCACTATGGTGGTAGATAGTGGTTTGAA
GGATGATTTGGTGTTGGGAGATCCTACTGCGCCAAGGTTTGTGTTGTGGAATGGGAAA
TTGAGGCCGGTTCCATCGAAGCTAACAGACTTACCGTTCTTTGATTTGATGAGTATTGG
TGGGAAGATTAGAGCTGGTTTTGGTGCACTTGGCATTCGACCGTCACCTCCAGGTCGT
GAAGAATCTGTGGAGGAGTTTGTACGGCGTAACCTCGGTGATGAGGTTTTTGAGCGCC
TGATTGAACCGTTTTGTTCAGGTGTTTATGCTGGTGATCCTTCAAAACTGAGCATGAAAG
CAGCGTTTGGGAAGGTTTGGAAACTAGAGCAAAATGGTGGAAGCATAATAGGTGGTAC
TTTTAAGGCAATTCAGGAGAGGAAAAACGCTCCCAAGGCAGAACGAGACCCGCGCCTG
CCAAAACCACAGGGCCAAACAGTTGGTTCTTTCAGGAAGGGACTTCGAATGTTGCCAG
AAGCAATATCTGCAAGATTAGGTAGCAAAGTTAAGTTGTCTTGGAAGCTCCTTGGTATC
ACTAAGCTGGAGAGCGGAGGATACAACTTAACATATGAGACTCCAGATGGTTTAGTTTC
CGTGCAGAGCAAAAGTGTTGTAATGACGGTGCCATCTCATGTTGCAAGTGGTCTCTTGC
GCCCTCTTTCTGAATCTGCTGCAAATGCACTCTCAAAACTATATTACCCACCAGTTGCAG
CAGTATCTATCTCGTACCCGAAAGAAGCAATCCGAACAGAATGTTTGATAGATGGTGAA
CTAAAGGGTTTTGGGCAATTGCATCCACGCACGCAAGGAGTTGAAACATTAGGAACTAT
CTACAGCTCCTCACTCTTTCCAAATCGCGCACCGCCCGGAAGAATTTTGCTGTTGAACG
TTATTGGCGGGTCTACAAACACCGGAATTCTGTCCAAGTCTGAAGGTGAGTTAGTGGAA
GCAGTTGACAGAGATTTGAGGAAAATGCTAATTAAGCCTAATTCGACCGATCCACTTAA
ATTAGGAGTTAGGGTATGGCCTCAAGCCATTCCTCAGTTTCTAGTTGGTCACTTTGATAT
CCTTGACACGGCTAAATCATCTCTAACGTCTTCGGGCTACGAAGGGCTATTTTTGGGTG
GCAATTACGTCGCTGGTGTAGCCTTAGGCCGGTGTGTAGAAGGCGCATATGAAACCGC
GATTGAGGTCAACAACTTCATGTCACGGTACGCTTACAAGTAAGGTACCGAGCTCGAAT
TTCCCCGATCGTTCAAACATTTGGCAATAAAGTTTCTTAAGATTGAATCCTGTTGCCGGT
CTTGCGATGATTATCATATAATTTCTGTTGAATTACGTTAAGCATGTAATAATTAACATGT
AATGCATGACGTTATTTATGAGATGGGTTTTTATGATTAGAGTCCCGCAATTATACATTT
AATACGCGATAGAAAACAAAATATAGCGCGCAAACTAGGATAAATTATCGCGCGCGGTG
TCATCTATGTTACTAGATCGGGAATTCCTAGAGATCATGAGCGGAGAATTAAGGGAGTC
ACGTTATGACCCCCGCCGATGACGCGGGACAAGCCGTTTTACGTTTGGAACTGACAGA
ACCGCAACGTTGAAGGAGCCACTCAGCCGCGGGTTTCTGGAGTTTAATGAGCTAAGCA
CATACGTCAGAAACCATTATTGCGCGTTCAAAAGTCGCCTAAGGTCACTATCAGCTAGC
AAATATTTCTTGTCAAAAATGCTCCACTGACGTTCCATAAATTCCCCTCGGTATCCAATT
AGAGTCTCATATTCACTCTCAATCCAAATAATCTGCACCGGATCTGGATCGTTTCGCATG
ATTGAACAAGATGGATTGCACGCAGGTTCTCCGGCCGCTTGGGTGGAGAGGCTATTCG
GCTATGACTGGGCACAACAGACAATCGGCTGCTCTGATGCCGCCGTGTTCCGGCTGTC
AGCGCAGGGGCGCCCGGTTCTTTTTGTCAAGACCGACCTGTCCGGTGCCCTGAATGAA
CTGCAGGACGAGGCAGCGCGGCTATCGTGGCTGGCCACGACGGGCGTTCCTTGCGCA
GCTGTGCTCGACGTTGTCACTGAAGCGGGAAGGGACTGGCTGCTATTGGGCGAAGTG
CCGGGGCAGGATCTCCTGTCATCTCACCTTGCTCCTGCCGAGAAAGTATCCATCATGG
CTGATGCAATGCGGCGGCTGCATACGCTTGATCCGGCTACCTGCCCATTCGACCACCA
AGCGAAACATCGCATCGAGCGAGCACGTACTCGGATGGAAGCCGGTCTTGTCGATCAG
GATGATCTGGACGAAGAGCATCAGGGGCTCGCGCCAGCCGAACTGTTCGCCAGGCTC
AAGGCGCGCATGCCCGACGGCGAGGATCTCGTCGTGACCCATGGCGATGCCTGCTTG
CCGAATATCATGGTGGAAAATGGCCGCTTTTCTGGATTCATCGACTGTGGCCGGCTGG
GTGTGGCGGACCGCTATCAGGACATAGCGTTGGCTACCCGTGATATTGCTGAAGAGCT
TGGCGGCGAATGGGCTGACCGCTTCCTCGTGCTTTACGGTATCGCCGCTCCCGATTCG
CAGCGCATCGCCTTCTATCGCCTTCTTGACGAGTTCTTCTGAGCGGGACTCTGGGGTT
CGAAATGACCGACCAAGCGACGCCCAACCTGCCATCACGAGATTTCGATTCCACCGCC
GCCTTCTATGAAAGGTTGGGCTTCGGAATCGTTTTCCGGGACGCCGGCTGGATGATCC
TCCAGCGCGGGGATCTCATGCTGGAGTTCTTCGCCCACGGGATCTCTGCGGAACAGG
CGGTCGAAGGTGCCGATATCATTACGACAGCAACGGCCGACAAGCACAACGCCACGAT
CCTGAGCGACAATATGATCGGGCCCGGCGTCCACATCAACGGCGTCGGCGGCGACTG
CCCAGGCAAGACCGAGATGCACCGCGATATCTTGCTGCGTTCGGATATTTTCGTGGAG
TTCCCGCCACAGACCCGGATGATCCCCGATCGTTCAAACATTTGGCAATAAAGTTTCTT
AAGATTGAATCCTGTTGCCGGTCTTGCGATGATTATCATATAATTTCTGTTGAATTACGT
TAAGCATGTAATAATTAACATGTAATGCATGACGTTATTTATGAGATGGGTTTTTATGATT
AGAGTCCCGCAATTATACATTTAATACGCGATAGAAAACAAAATATAGCGCGCAAACTA
GGATAAATTATCGCGCGCGGTGTCATCTATGTTACTAGATCGGGCCTCCTGTCAATGCT
GGCGGCGGCTCTGGTGGTGGTTCTGGTGGCGGCTCTGAGGGTGGTGGCTCTGAGGG
TGGCGGTTCTGAGGGTGGCGGCTCTGAGGGAGGCGGTTCCGGTGGTGGCTCTGGTTC
CGGTGATTTTGATTATGAAAAGATGGCAAACGCTAATAAGGGGGCTATGACCGAAAATG
CCGATGAAAACGCGCTACAGTCTGACGCTAAAGGCAAACTTGATTCTGTCGCTACTGAT
TACGGTGCTGCTATCGGCCTGAATGGCGCCCGCTCCTTTCGCTTTCTTCCCTTCCTTTC
TCGCCACGTTCGCCGGCTTTCCCCGTCAAGCTCTAAATCGGGGGCTCCCTTTAGGGTT
CCGATTTAGTGCTTTACGGCACCTCGACCCCAAAAAACTTGATTTGGGTGATGGTTCAC
GTAGTGGGCCATCGCCCTGATAGACGGTTTTTCGCCCTTTGACGTTGGAGTCCACGTT
CTTTAATAGTGGACTCTTGTTCCAAACTGGAACAACACTCAACCCTATCTCGGGCTATTC
TTTTGATTTATAAGGGATTTTGCCGATTTCGGAACCACCATCAAACAGGATTTTCGCCTG
CTGGGGCAAACCAGCGTGGACCGCTTGCTGCAACTCTCTCAGGGCCAGGCGGTGAAG
GGCAATCAGCTGTTGCCCGTCTCACTGGTGAAAAGAAAAACCACCCCAGTACATTAAAA
ACGTCCGCAATGTGTTATTAAGTTGTCTAAGCGTCAATTTGTTTACACCACAATATATCC
TGCCACCAGCCAGCCAACAGCTCCCCGACCGGCAGCTCGGCACAAAATCACCACTCG
ATACAGGCAGCCCATCAGTCCGGGACGGCGTCAGCGGGAGAGCCGTTGTAAGGCGGC
AGACTTTGCTCATGTTACCGATGCTATTCGGAAGAACGGCAACTAAGCTGCCGGGTTTG
AAACACGGATGATCTCGCGGAGGGTAGCATGTTGATTGTAACGATGACAGAGCGTTGC
TGCCTGTGATCAAATATCATCTCCCTCGCAGAGATCCGAATTATCAGCCTTCTTATTCAT
TTCTCGCTTAACCGTGACAGGCTGTCGATCTTGAGAACTATGCCGACATAATAGGAAAT
CGCTGGATAAAGCCGCTGAGGAAGCTGAGTGGCGCTATTTCTTTAGAAGTGAACGTTG
ACGATATCAACTCCCCTATCCATTGCTCACCGAATGGTACAGGTCGGGGACCCGAAGT
TCCGACTGTCGGCCTGATGCATCCCCGGCTGATCGACCCCAGATCTGGGGCTGAGAAA
GCCCAGTAAGGAAACAACTGTAGGTTCGAGTCGCGAGATCCCCCGGAACCAAAGGAAG
TAGGTTAAACCCGCTCCGATCAGGCCGAGCCACGCCAGGCCGAGAACATTGGTTCCTG
TAGGCATCGGGATTGGCGGATCAAACACTAAAGCTACTGGAACGAGCAGAAGTCCTCC
GGCCGCCAGTTGCCAGGCGGTAAAGGTGAGCAGAGGCACGGGAGGTTGCCACTTGCG
GGTCAGCACGGTTCCGAACGCCATGGAAACCGCCCCCGCCAGGCCCGCTGCGACGCC
GACAGGATCTAGCGCTGCGTTTGGTGTCAACACCAACAGCGCCACGCCCGCAGTTCCG
CAAATAGCCCCCAGGACCGCCATCAATCGTATCGGGCTACCTAGCAGAGCGGCAGAGA
TGAACACGACCATCAGCGGCTGCACAGCGCCTACCGTCGCCGCGACCCCGCCCGGCA
GGCGGTAGACCGAAATAAACAACAAGCTCCAGAATAGCGAAATATTAAGTGCGCCGAG
GATGAAGATGCGCATCCACCAGATTCCCGTTGGAATCTGTCGGACGATCATCACGAGC
AATAAACCCGCCGGCAACGCCCGCAGCAGCATACCGGCGACCCCTCGGCCTCGCTGT
TCGGGCTCCACGAAAACGCCGGACAGATGCGCCTTGTGAGCGTCCTTGGGGCCGTCC
TCCTGTTTGAAGACCGACAGCCCAATGATCTCGCCGTCGATGTAGGCGCCGAATGCCA
CGGCATCTCGCAACCGTTCAGCGAACGCCTCCATGGGCTTTTTCTCCTCGTGCTCGTA
AACGGACCCGAACATCTCTGGAGCTTTCTTCAGGGCCGACAATCGGATCTCGCGGAAA
TCCTGCACGTCGGCCGCTCCAAGCCGTCGAATCTGAGCCTTAATCACAATTGTCAATTT
TAATCCTCTGTTTATCGGCAGTTCGTAGAGCGCGCCGTGCGTCCCGAGCGATACTGAG
CGAAGCAAGTGCGTCGAGCAGTGCCCGCTTGTTCCTGAAATGCCAGTAAAGCGCTGGC
TGCTGAACCCCCAGCCGGAACTGACCCCACAAGGCCCTAGCGTTTGCAATGCACCAGG
TCATCATTGACCCAGGCGTGTTCCACCAGGCCGCTGCCTCGCAACTCTTCGCAGGCTT
CGCCGACCTGCTCGCGCCACTTCTTCACGCGGGTGGAATCCGATCCGCACATGAGGC
GGAAGGTTTCCAGCTTGAGCGGGTACGGCTCCCGGTGCGAGCTGAAATAGTCGAACAT
CCGTCGGGCCGTCGGCGACAGCTTGCGGTACTTCTCCCATATGAATTTCGTGTAGTGG
TCGCCAGCAAACAGCACGACGATTTCCTCGTCGATCAGGACCTGGCAACGGGACGTTT
TCTTGCCACGGTCCAGGACGCGGAAGCGGTGCAGCAGCGACACCGATTCCAGGTGCC
CAACGCGGTCGGACGTGAAGCCCATCGCCGTCGCCTGTAGGCGCGACAGGCATTCCT
CGGCCTTCGTGTAATACCGGCCATTGATCGACCAGCCCAGGTCCTGGCAAAGCTCGTA
GAACGTGAAGGTGATCGGCTCGCCGATAGGGGTGCGCTTCGCGTACTCCAACACCTG
CTGCCACACCAGTTCGTCATCGTCGGCCCGCAGCTCGACGCCGGTGTAGGTGATCTTC
ACGTCCTTGTTGACGTGGAAAATGACCTTGTTTTGCAGCGCCTCGCGCGGGATTTTCTT
GTTGCGCGTGGTGAACAGGGCAGAGCGGGCCGTGTCGTTTGGCATCGCTCGCATCGT
GTCCGGCCACGGCGCAATATCGAACAAGGAAAGCTGCATTTCCTTGATCTGCTGCTTC
GTGTGTTTCAGCAACGCGGCCTGCTTGGCCTCGCTGACCTGTTTTGCCAGGTCCTCGC
CGGCGGTTTTTCGCTTCTTGGTCGTCATAGTTCCTCGCGTGTCGATGGTCATCGACTTC
GCCAAACCTGCCGCCTCCTGTTCGAGACGACGCGAACGCTCCACGGCGGCCGATGGC
GCGGGCAGGGCAGGGGGAGCCAGTTGCACGCTGTCGCGCTCGATCTTGGCCGTAGCT
TGCTGGACCATCGAGCCGACGGACTGGAAGGTTTCGCGGGGCGCACGCATGACGGTG
CGGCTTGCGATGGTTTCGGCATCCTCGGCGGAAAACCCCGCGTCGATCAGTTCTTGCC
TGTATGCCTTCCGGTCAAACGTCCGATTCATTCACCCTCCTTGCGGGATTGCCCCGACT
CACGCCGGGGCAATGTGCCCTTATTCCTGATTTGACCCGCCTGGTGCCTTGGTGTCCA
GATAATCCACCTTATCGGCAATGAAGTCGGTCCCGTAGACCGTCTGGCCGTCCTTCTC
GTACTTGGTATTCCGAATCTTGCCCTGCACGAATACCAGCGACCCCTTGCCCAAATACT
TGCCGTGGGCCTCGGCCTGAGAGCCAAAACACTTGATGCGGAAGAAGTCGGTGCGCT
CCTGCTTGTCGCCGGCATCGTTGCGCCACATCTAGGTACTAAAACAATTCATCCAGTAA
AATATAATATTTTATTTTCTCCCAATCAGGCTTGATCCCCAGTAAGTCAAAAAATAGCTC
GACATACTGTTCTTCCCCGATATCCTCCCTGATCGACCGGACGCAGAAGGCAATGTCAT
ACCACTTGTCCGCCCTGCCGCTTCTCCCAAGATCAATAAAGCCACTTACTTTGCCATCT
TTCACAAAGATGTTGCTGTCTCCCAGGTCGCCGTGGGAAAAGACAAGTTCCTCTTCGG
GCTTTTCCGTCTTTAAAAAATCATACAGCTCGCGCGGATCTTTAAATGGAGTGTCTTCTT
CCCAGTTTTCGCAATCCACATCGGCCAGATCGTTATTCAGTAAGTAATCCAATTCGGCT
AAGCGGCTGTCTAAGCTATTCGTATAGGGACAATCCGATATGTCGATGGAGTGAAAGA
GCCTGATGCACTCCGCATACAGCTCGATAATCTTTTCAGGGCTTTGTTCATCTTCATACT
CTTCCGAGCAAAGGACGCCATCGGCCTCACTCATGAGCAGATTGCTCCAGCCATCATG
CCGTTCAAAGTGCAGGACCTTTGGAACAGGCAGCTTTCCTTCCAGCCATAGCATCATGT
CCTTTTCCCGTTCCACATCATAGGTGGTCCCTTTATACCGGCTGTCCGTCATTTTTAAAT
ATAGGTTTTCATTTTCTCCCACCAGCTTATATACCTTAGCAGGAGACATTCCTTCCGTAT
CTTTTACGCAGCGGTATTTTTCGATCAGTTTTTTCAATTCCGGTGATATTCTCATTTTAGC
CATTTATTATTTCCTTCCTCTTTTCTACAGTATTTAAAGATACCCCAAGAAGCTAATTATA
ACAAGACGAACTCCAATTCACTGTTCCTTGCATTCTAAAACCTTAAATACCAGAAAACAG
CTTTTTCAAAGTTGTTTTCAAAGTTGGCGTATAACATAGTATCGACGGAGCCGATTTTGA
AACCACAATTATGGGTGATGCTGCCAACTTACTGATTTAGTGTATGATGGTGTTTTTGAG
GTGCTCCAGTGGCTTCTGTTTCTATCAGCTGTCCCTCCTGTTCAGCTACTGACGGGGTG
GTGCGTAACGGCAAAAGCACCGCCGGACATCAGCGCTATCTCTGCTCTCACTGCCGTA
AAACATGGCAACTGCAGTTCACTTACACCGCTTCTCAACCCGGTACGCACCAGAAAATC
ATTGATATGGCCATGAATGGCGTTGGATGCCGGGCAACAGCCCGCATTATGGGCGTTG
GCCTCAACACGATTTTACGTCACTTAAAAAACTCAGGCCGCAGTCGGTAACCTCGCGCA
TACAGCCGGGCAGTGACGTCATCGTCTGCGCGGAAATGGACGAACAGTGGGGCTATG
TCGGGGCTAAATCGCGCCAGCGCTGGCTGTTTTACGCGTATGACAGTCTCCGGAAGAC
GGTTGTTGCGCACGTATTCGGTGAACGCACTATGGCGACGCTGGGGCGTCTTATGAGC
CTGCTTCACCCTTTGACGTGGTGATATGGATGACGGATGGCTGGCCGCTGTATGAATC
CCGCCTGAAGGGAAAGCTGCACGTAATCAGCAAGCGATATACGCAGCGAATTGAGCGG
CATAACCTGAATCTGAGGCAGCACCTGGCACGGCTGGGACGGAAGTCGCTGTCGTTCT
CAAAATCGGTGGAGCTGCATGACAAAGTCATCGGGCATTATCTGAACATAAAACACTAT
CAATAAGTTGGAGTCATTACCCAATTATGATAGAATTTACAAGCTATAAGGTTATTGTCC
TGGGTTTCAAGCATTAGTCCATGCAAGTTTTTATGCTTTGCCCATTCTATAGATATATTG
ATAAGCGCGCTGCCTATGCCTTGCCCCCTGAAATCCTTACATACGGCGATATCTTCTAT
ATAAAAGATATATTATCTTATCAGTATTGTCAATATATTCAAGGCAATCTGCCTCCTCATC
CTCTTCATCCTCTTCGTCTTGGTAGCTTTTTAAATATGGCGCTTCATAGAGTAATTCTGT
AAAGGTCCAATTCTCGTTTTCATACCTCGGTATAATCTTACCTATCACCTCAAATGGTTC
GCTGGGTTTATCGCACCCCCGAACACGAGCACGGCACCCGCGACCACTATGCCAAGA
ATGCCCAAGGTAAAAATTGCCGGCCCCGCCATGAAGTCCGTGAATGCCCCGACGGCC
GAAGTGAAGGGCAGGCCGCCACCCAGGCCGCCGCCCTCACTGCCCGGCACCTGGTC
GCTGAATGTCGATGCCAGCACCTGCGGCACGTCAATGCTTCCGGGCGTCGCGCTCGG
GCTGATCGCCCATCCCGTTACTGCCCCGATCCCGGCAATGGCAAGGACTGCCAGCGC
TGCCATTTTTGGGGTGAGGCCGTTCGCGGCCGAGGGGCGCAGCCCCTGGGGGGATG
GGAGGCCCGCGTTAGCGGGCCGGGAGGGTTCGAGAAGGGGGGGCACCCCCCTTCGG
CGTGCGCGGTCACGCGCACAGGGCGCAGCCCTGGTTAAAAACAAGGTTTATAAATATT
GGTTTAAAAGCAGGTTAAAAGACAGGTTAGCGGTGGCCGAAAAACGGGCGGAAACCCT
TGCAAATGCTGGATTTTCTGCCTGTGGACAGCCCCTCAAATGTCAATAGGTGCGCCCCT
CATCTGTCAGCACTCTGCCCCTCAAGTGTCAAGGATCGCGCCCCTCATCTGTCAGTAGT
CGCGCCCCTCAAGTGTCAATACCGCAGGGCACTTATCCCCAGGCTTGTCCACATCATC
TGTGGGAAACTCGCGTAAAATCAGGCGTTTTCGCCGATTTGCGAGGCTGGCCAGCTCC
ACGTCGCCGGCCGAAATCGAGCCTGCCCCTCATCTGTCAACGCCGCGCCGGGTGAGT
CGGCCCCTCAAGTGTCAACGTCCGCCCCTCATCTGTCAGTGAGGGCCAAGTTTTCCGC
GAGGTATCCACAACGCCGGCGGCCGCGGTGTCTCGCACACGGCTTCGACGGCGTTTC
TGGCGCGTTTGCAGGGCCATAGACGGCCGCCAGCCCAGCGGCGAGGGCAACCAGCC
CGGTGAGCGTCGGAAAGGCGCTCGGTCTTGCCTTGCTCGTCGGTGATGTACTTCACCA
GCTCCGCGAAGTCGCTCTTCTTGATGGAGCGCATGGGGACGTGCTTGGCAATCACGC
GCACCCCCCGGCCGTTTTAGCGGCTAAAAAAGTCATGGCTCTGCCCTCGGGCGGACC
ACGCCCATCATGACCTTGCCAAGCTCGTCCTGCTTCTCTTCGATCTTCGCCAGCAGGG
CGAGGATCGTGGCATCACCGAACCGCGCCGTGCGCGGGTCGTCGGTGAGCCAGAGTT
TCAGCAGGCCGCCCAGGCGGCCCAGGTCGCCATTGATGCGGGCCAGCTCGCGGACG
TGCTCATAGTCCACGACGCCCGTGATTTTGTAGCCCTGGCCGACGGCCAGCAGGTAGG
CCGACAGGCTCATGCCGGCCGCCGCCGCCTTTTCCTCAATCGCTCTTCGTTCGTCTGG
AAGGCAGTACACCTTGATAGGTGGGCTGCCCTTCCTGGTTGGCTTGGTTTCATCAGCC
ATCCGCTTGCCCTCATCTGTTACGCCGGCGGTAGCCGGCCAGCCTCGCAGAGCAGGA
TTCCCGTTGAGCACCGCCAGGTGCGAATAAGGGACAGTGAAGAAGGAACACCCGCTC
GCGGGTGGGCCTACTTCACCTATCCTGCCCGGCTGACGCCGTTGGATACACCAAGGAA
AGTCTACACGAACCCTTTGGCAAAATCCTGTATATCGTGCGAAAAAGGATGGATATACC
GAAAAAATCGCTATAATGACCCCGAAGCAGGGTTATGCAGCGGAAAAGCGCCACGCTT
CCCGAAGGGAGAAAGGCGGACAGGTATCCGGTAAGCGGCAGGGTCGGAACAGGAGA
GCGCACGAGGGAGCTTCCAGGGGGAAACGCCTGGTATCTTTATAGTCCTGTCGGGTTT
CGCCACCTCTGACTTGAGCGTCGATTTTTGTGATGCTCGTCAGGGGGGCGGAGCCTAT
GGAAAAACGCCAGCAACGCGGCCTTTTTACGGTTCCTGGCCTTTTGCTGGCCTTTTGCT
CACATGTTCTTTCCTGCGTTATCCCCTGATTCTGTGGATAACCGTATTACCGCCTTTGAG
TGAGCTGATACCGCTCGCCGCAGCCGAACGACCGAGCGCAGCGAGTCAGTGAGCGAG
GAAGCGGAAGAGCGCCAGAAGGCCGCCAGAGAGGCCGAGCGCGGCCGTGAGGCTTG
GACGCTAGGGCAGGGCATGAAAAAGCCCGTAGCGGGCTGCTACGGGCGTCTGACGCG
GTGGAAAGGGGGAGGGGATGTTGTCTACATGGCTCTGCTGTAGTGAGTGGGTTGCGC
TCCGGCAGCGGTCCTGATCAATCGTCACCCTTTCTCGGTCCTTCAACGTTCCTGACAAC
GAGCCTCCTTTTCGCCAATCCATCGACAATCACCGCGAGTCCCTGCTCGAACGCTGCG
TCCGGACCGGCTTCGTCGAAGGCGTCTATCGCGGCCCGCAACAGCGGCGAGAGCGGA
GCCTGTTCAACGGTGCCGCCGCGCTCGCCGGCATCGCTGTCGCCGGCCTGCTCCTCA
AGCACGGCCCCAACAGTGAAGTAGCTGATTGTCATCAGCGCATTGACGGCGTCCCCGG
CCGAAAAACCCGCCTCGCAGAGGAAGCGAAGCTGCGCGTCGGCCGTTTCCATCTGCG
GTGCGCCCGGTCGCGTGCCGGCATGGATGCGCGCGCCATCGCGGTAGGCGAGCAGC
GCCTGCCTGAAGCTGCGGGCATTCCCGATCAGAAATGAGCGCCAGTCGTCGTCGGCT
CTCGGCACCGAATGCGTATGATTCTCCGCCAGCATGGCTTCGGCCAGTGCGTCGAGCA
GCGCCCGCTTGTTCCTGAAGTGCCAGTAAAGCGCCGGCTGCTGAACCCCCAACCGTTC
CGCCAGTTTGCGTGTCGTCAGACCGTCTACGCCGACCTCGTTCAACAGGTCCAGGGCG
GCACGGATCACTGTATTCGGCTGCAACTTTGTCATGCTTGACACTTTATCACTGATAAAC
ATAATATGTCCACCAACTTATCAGTGATAAAGAATCCGCGCGTTCAATCGGACCAGCGG
AGGCTGGTCCGGAGGCCAGACGTGAAACCCAACATACCCCTGATCGTAATTCTGAGCA
CTGTCGCGCTCGACGCTGTCGGCATCGGCCTGATTATGCCGGTGCTGCCGGGCCTCC
TGCGCGATCTGGTTCACTCGAACGACGTCACCGCCCACTATGGCATTCTGCTGGCGCT
GTATGCGTTGGTGCAATTTGCCTGCGCACCTGTGCTGGGCGCGCTGTCGGATCGTTTC
GGGCGGCGGCCAATCTTGCTCGTCTCGCTGGCCGGCGCCAGATCTGGGGAACCCTGT
GGTTGGCATGCACATACAAATGGACGAACGGATAAACCTTTTCACGCCCTTTTAAATAT
CCGATTATTCTAATAAACGCTCTTTTCTCTTAGGTTTACCCGCCAATATATCCTGTCAAA
CACTGATAGTTTAAACTGAAGGCGGGAAACGACAATCTGATCTCTAGGA
(without transit peptides)
Arabidopsis thaliana PPO1 (SEQ ID NO: 153)
SVAGGPTVGSSKIEGGGGTTITTDCVIVGGGISGLCIAQALATKHPDAAPNLIVTEAKDRVGG
NIITREENGFLWEEGPNSFQPSDPMLTMVVDSGLKDDLVLGDPTAPRFVLWNGKLRPVPSK
LTDLPFFDLMSIGGKIRAGFGALGIRPSPPGREESVEEFVRRNLGDEVFERLIEPFCSGVYA
GDPSKLSMKAAFGKVWKLEQNGGSIIGGTFKAIQERKNAPKAERDPRLPKPQGQTVGSFR
KGLRMLPEAISARLGSKVKLSWKLSGITKLESGGYNLTYETPDGLVSVQSKSVVMTVPSHV
ASGLLRPLSESAANALSKLYYPPVAAVSISYPKEAIRTECLIDGELKGFGQLHPRTQGVETLG
TIYSSSLFPNRAPPGRILLLNYIGGSTNTGILSKSEGELVEAVDRDLRKMLIKPNSTDPLKLGV
RVWPQAIPQFLVGHFDILDTAKSSLTSSGYEGLFLGGNYVAGVALGRCVEGAYETAIEVNNF
MSRYAYK*
Setaria italica PPO1 (SEQ ID NO: 154)
AAVAGGAAEAPASAGARVSADCVVVGGGISGLCTAQALATKHGVGDVLVTEARARPGGNI
TTVERPDEGYLWEEGPNSFQPSDPVLTMAVDSGLKDDLVFGDPNAPRFVLWEGKLRPVPS
KPADLPFFDLMSIPGKLRAGFGALGIRPPPPGREESVEEFVRRNLGAEVFERLIEPFCSGVY
AGDPSKLSMKAAFGKVWRLEEAGGSIIGGTIKTIQERGKNPKPPRDPRLPTPKGQTVASFR
KGLAMLPNAITSSLGSKVKLSWKLTSITKSDGMGYVLVYETPEGVVSVQAKSVIMTIPSYVA
SDILRPLSSDAADALSRFYYPPVAAVTISYPKEAIRKECLIDGELQGFGQLHPRSQGVETLGT
IYSSSLFPNRAPAGRVLLLNYIGGATNTGIVSKSASELVEAVDRDLRKMLINPSAVDPLVLGV
RVWPQAIPQFLVGHLDLLEAAKSSLDRGGYDGLFLGGNYVAGVALGRCVEGAYESASQIS
DFLTKYAYK*
Arabidopsis thaliana PPO1 G30A (SEQ ID NO: 155)
SVAGGPTVGSSKIEGGGGTTITTDCVIVGAGISGLCIAQALATKHPDAAPNLIVTEAKDRVGG
NIITREENGFLWEEGPNSFQPSDPMLTMVVDSGLKDDLVLGDPTAPRFVLWNGKLRPVPSK
LTDLPFFDLMSIGGKIRAGFGALGIRPSPPGREESVEEFVRRNLGDEVFERLIEPFCSGVYA
GDPSKLSMKAAFGKVWKLEQNGGSIIGGTFKAIQERKNAPKAERDPRLPKPQGQTVGSFR
KGLRMLPEAISARLGSKVKLSWKLSGITKLESGGYNLTYETPDGLVSVQSKSVVMTVPSHV
ASGLLRPLSESAANALSKLYYPPVAAVSISYPKEAIRTECLIDGELKGFGQLHPRTQGVETLG
TIYSSSLFPNRAPPGRILLLNYIGGSTNTGILSKSEGELVEAVDRDLRKMLIKPNSTDPLKLGV
RVWPQAIPQFLVGHFDILDTAKSSLTSSGYEGLFLGGNYVAGVALGRCVEGAYETAIEVNNF
MSRYAYK*
Arabidopsis thaliana PPO1 V54I (SEQ ID NO: 156)
SVAGGPTVGSSKIEGGGGTTITTDCVIVGGGISGLCIAQALATKHPDAAPNLIITEAKDRVGG
NIITREENGFLWEEGPNSFQPSDPMLTMVVDSGLKDDLVLGDPTAPRFVLWNGKLRPVPSK
LTDLPFFDLMSIGGKIRAGFGALGIRPSPPGREESVEEFVRRNLGDEVFERLIEPFCSGVYA
GDPSKLSMKAAFGKVWKLEQNGGSIIGGTFKAIQERKNAPKAERDPRLPKPQGQTVGSFR
KGLRMLPEAISARLGSKVKLSWKLSGITKLESGGYNLTYETPDGLVSVQSKSVVMTVPSHV
ASGLLRPLSESAANALSKLYYPPVAAVSISYPKEAIRTECLIDGELKGFGQLHPRTQGVETLG
TIYSSSLFPNRAPPGRILLLNYIGGSTNTGILSKSEGELVEAVDRDLRKMLIKPNSTDPLKLGV
RVWPQAIPQFLVGHFDILDTAKSSLTSSGYEGLFLGGNYVAGVALGRCVEGAYETAIEVNNF
MSRYAYK*
Arabidopsis thaliana PPO1 E77D (SEQ ID NO: 157)
SVAGGPTVGSSKIEGGGGTTITTDCVIVGGGISGLCIAQALATKHPDAAPNLIVTEAKDRVGG
NIITREENGFLWEDGPNSFQPSDPMLTMVVDSGLKDDLVLGDPTAPRFVLWNGKLRPVPS
KLTDLPFFDLMSIGGKIRAGFGALGIRPSPPGREESVEEFVRRNLGDEVFERLIEPFCSGVY
AGDPSKLSMKAAFGKVWKLEQNGGSIIGGTFKAIQERKNAPKAERDPRLPKPQGQTVGSF
RKGLRMLPEAISARLGSKVKLSWKLSGITKLESGGYNLTYETPDGLVSVQSKSVVMTVPSH
VASGLLRPLSESAANALSKLYYPPVAAVSISYPKEAIRTECLIDGELKGFGQLHPRTQGVETL
GTIYSSSLFPNRAPPGRILLLNYIGGSTNTGILSKSEGELVEAVDRDLRKMLIKPNSTDPLKLG
VRVWPQAIPQFLVGHFDILDTAKSSLTSSGYEGLFLGGNYVAGVALGRCVEGAYETAIEVN
NFMSRYAYK*
Arabidopsis thaliana PPO1 E77F (SEQ ID NO: 158)
SVAGGPTVGSSKIEGGGGTTITTDCVIVGGGISGLCIAQALATKHPDAAPNLIVTEAKDRVGG
NIITREENGFLWEFGPNSFQPSDPMLTMVVDSGLKDDLVLGDPTAPRFVLWNGKLRPVPSK
LTDLPFFDLMSIGGKIRAGFGALGIRPSPPGREESVEEFVRRNLGDEVFERLIEPFCSGVYA
GDPSKLSMKAAFGKVWKLEQNGGSIIGGTFKAIQERKNAPKAERDPRLPKPQGQTVGSFR
KGLRMLPEAISARLGSKVKLSWKLSGITKLESGGYNLTYETPDGLVSVQSKSVVMTVPSHV
ASGLLRPLSESAANALSKLYYPPVAAVSISYPKEAIRTECLIDGELKGFGQLHPRTQGVETLG
TIYSSSLFPNRAPPGRILLLNYIGGSTNTGILSKSEGELVEAVDRDLRKMLIKPNSTDPLKLGV
RVWPQAIPQFLVGHFDILDTAKSSLTSSGYEGLFLGGNYVAGVALGRCVEGAYETAIEVNNF
MSRYAYK*
Arabidopsis thaliana PPO1 V93I (SEQ ID NO: 159)
SVAGGPTVGSSKIEGGGGTTITTDCVIVGGGISGLCIAQALATKHPDAAPNLIVTEAKDRVGG
NIITREENGFLWEEGPNSFQPSDPMLTMVIDSGLKDDLVLGDPTAPRFVLWNGKLRPVPSK
LTDLPFFDLMSIGGKIRAGFGALGIRPSPPGREESVEEFVRRNLGDEVFERLIEPFCSGVYA
GDPSKLSMKAAFGKVWKLEQNGGSIIGGTFKAIQERKNAPKAERDPRLPKPQGQTVGSFR
KGLRMLPEAISARLGSKVKLSWKLSGITKLESGGYNLTYETPDGLVSVQSKSVVMTVPSHV
ASGLLRPLSESAANALSKLYYPPVAAVSISYPKEAIRTECLIDGELKGFGQLHPRTQGVETLG
TIYSSSLFPNRAPPGRILLLNYIGGSTNTGILSKSEGELVEAVDRDLRKMLIKPNSTDPLKLGV
RVWPQAIPQFLVGHFDILDTAKSSLTSSGYEGLFLGGNYVAGVALGRCVEGAYETAIEVNNF
MSRYAYK*
Arabidopsis thaliana PPO1 V93L (SEQ ID NO: 160)
SVAGGPTVGSSKIEGGGGTTITTDCVIVGGGISGLCIAQALATKHPDAAPNLIVTEAKDRVGG
NIITREENGFLWEEGPNSFQPSDPMLTMVLDSGLKDDLVLGDPTAPRFVLWNGKLRPVPSK
LTDLPFFDLMSIGGKIRAGFGALGIRPSPPGREESVEEFVRRNLGDEVFERLIEPFCSGVYA
GDPSKLSMKAAFGKVWKLEQNGGSIIGGTFKAIQERKNAPKAERDPRLPKPQGQTVGSFR
KGLRMLPEAISARLGSKVKLSWKLSGITKLESGGYNLTYETPDGLVSVQSKSVVMTVPSHV
ASGLLRPLSESAANALSKLYYPPVAAVSISYPKEAIRTECLIDGELKGFGQLHPRTQGVETLG
TIYSSSLFPNRAPPGRILLLNYIGGSTNTGILSKSEGELVEAVDRDLRKMLIKPNSTDPLKLGV
RVWPQAIPQFLVGHFDILDTAKSSLTSSGYEGLFLGGNYVAGVALGRCVEGAYETAIEVNNF
MSRYAYK*
Arabidopsis thaliana PPO1 P120Y (SEQ ID NO: 161)
SVAGGPTVGSSKIEGGGGTTITTDCVIVGGGISGLCIAQALATKHPDAAPNLIVTEAKDRVGG
NIITREENGFLWEEGPNSFQPSDPMLTMVVDSGLKDDLVLGDPTAPRFVLWNGKLRYVPSK
LTDLPFFDLMSIGGKIRAGFGALGIRPSPPGREESVEEFVRRNLGDEVFERLIEPFCSGVYA
GDPSKLSMKAAFGKVWKLEQNGGSIIGGTFKAIQERKNAPKAERDPRLPKPQGQTVGSFR
KGLRMLPEAISARLGSKVKLSWKLSGITKLESGGYNLTYETPDGLVSVQSKSVVMTVPSHV
ASGLLRPLSESAANALSKLYYPPVAAVSISYPKEAIRTECLIDGELKGFGQLHPRTQGVETLG
TIYSSSLFPNRAPPGRILLLNYIGGSTNTGILSKSEGELVEAVDRDLRKMLIKPNSTDPLKLGV
RVWPQAIPQFLVGHFDILDTAKSSLTSSGYEGLFLGGNYVAGVALGRCVEGAYETAIEVNNF
MSRYAYK*
Arabidopsis thaliana PPO1 K139H (SEQ ID NO: 162)
SVAGGPTVGSSKIEGGGGTTITTDCVIVGGGISGLCIAQALATKHPDAAPNLIVTEAKDRVGG
NIITREENGFLWEEGPNSFQPSDPMLTMVVDSGLKDDLVLGDPTAPRFVLWNGKLRPVPSK
LTDLPFFDLMSIGGHIRAGFGALGIRPSPPGREESVEEFVRRNLGDEVFERLIEPFCSGVYA
GDPSKLSMKAAFGKVWKLEQNGGSIIGGTFKAIQERKNAPKAERDPRLPKPQGQTVGSFR
KGLRMLPEAISARLGSKVKLSWKLSGITKLESGGYNLTYETPDGLVSVQSKSVVMTVPSHV
ASGLLRPLSESAANALSKLYYPPVAAVSISYPKEAIRTECLIDGELKGFGQLHPRTQGVETLG
TIYSSSLFPNRAPPGRILLLNYIGGSTNTGILSKSEGELVEAVDRDLRKMLIKPNSTDPLKLGV
RVWPQAIPQFLVGHFDILDTAKSSLTSSGYEGLFLGGNYVAGVALGRCVEGAYETAIEVNNF
MSRYAYK*
Arabidopsis thaliana PPO1 A142L (SEQ ID NO: 163)
SVAGGPTVGSSKIEGGGGTTITTDCVIVGGGISGLCIAQALATKHPDAAPNLIVTEAKDRVGG
NIITREENGFLWEEGPNSFQPSDPMLTMVVDSGLKDDLVLGDPTAPRFVLWNGKLRPVPSK
LTDLPFFDLMSIGGKIRLGFGALGIRPSPPGREESVEEFVRRNLGDEVFERLIEPFCSGVYA
GDPSKLSMKAAFGKVWKLEQNGGSIIGGTFKAIQERKNAPKAERDPRLPKPQGQTVGSFR
KGLRMLPEASARLGSKVKLSWKLSGITKLESGGYNLTYETPDGLVSVQSKSVVMTVPSHV
ASGLLRPLSESAANALSKLYYPPVAAVSISYPKEAIRTECLIDGELKGFGQLHPRTQGVETLG
TIYSSSLFPNRAPPGRILLLNYIGGSTNTGILSKSEGELVEAVDRDLRKMLIKPNSTDPLKLGV
RVWPQAIPQFLVGHFDILDTAKSSLTSSGYEGLFLGGNYVAGVALGRCVEGAYETAIEVNNF
MSRYAYK*
Arabidopsis thaliana PPO1 V164W (SEQ ID NO: 164)
SVAGGPTVGSSKIEGGGGTTITTDCVIVGGGISGLCIAQALATKHPDAAPNLIVTEAKDRVGG
NIITREENGFLWEEGPNSFQPSDPMLTMVVDSGLKDDLVLGDPTAPRFVLWNGKLRPVPSK
LTDLPFFDLMSIGGKIRAGFGALGIRPSPPGREESVEEFWRRNLGDEVFERLIEPFCSGVYA
GDPSKLSMKAAFGKVWKLEQNGGSIIGGTFKAIQERKNAPKAERDPRLPKPQGQTVGSFR
KGLRMLPEAISARLGSKVKLSWKLSGITKLESGGYNLTYETPDGLVSVQSKSVVMTVPSHV
ASGLLRPLSESAANALSKLYYPPVAAVSISYPKEAIRTECLIDGELKGFGQLHPRTQGVETLG
TIYSSSLFPNRAPPGRILLLNYIGGSTNTGILSKSEGELVEAVDRDLRKMLIKPNSTDPLKLGV
RVWPQAIPQFLVGHFDILDTAKSSLTSSGYEGLFLGGNYVAGVALGRCVEGAYETAIEVNNF
MSRYAYK*
Arabidopsis thaliana PPO1 V172G (SEQ ID NO: 165)
SVAGGPTVGSSKIEGGGGTTITTDCVIVGGGISGLCIAQALATKHPDAAPNLIVTEAKDRVGG
NIITREENGFLWEEGPNSFQPSDPMLTMVVDSGLKDDLVLGDPTAPRFVLWNGKLRPVPSK
LTDLPFFDLMSIGGKIRAGFGALGIRPSPPGREESVEEFVRRNLGDEGFERLIEPFCSGVYA
GDPSKLSMKAAFGKVWKLEQNGGSIIGGTFKAIQERKNAPKAERDPRLPKPQGQTVGSFR
KGLRMLPEAISARLGSKVKLSWKLSGITKLESGGYNLTYETPDGLVSVQSKSVVMTVPSHV
ASGLLRPLSESAANALSKLYYPPVAAVSISYPKEAIRTECLIDGELKGFGQLHPRTQGVETLG
TIYSSSLFPNRAPPGRILLLNYIGGSTNTGILSKSEGELVEAVDRDLRKMLIKPNSTDPLKLGV
RVWPQAIPQFLVGHFDILDTAKSSLTSSGYEGLFLGGNYVAGVALGRCVEGAYETAIEVNNF
MSRYAYK*
Arabidopsis thaliana PPO1 F173Y (SEQ ID NO: 166)
SVAGGPTVGSSKIEGGGGTTITTDCVIVGGGISGLCIAQALATKHPDAAPNLIVTEAKDRVGG
NIITREENGFLWEEGPNSFQPSDPMLTMVVDSGLKDDLVLGDPTAPRFVLWNGKLRPVPSK
LTDLPFFDLMSIGGKIRAGFGALGIRPSPPGREESVEEFVRRNLGDEVYERLIEPFCSGVYA
GDPSKLSMKAAFGKVWKLEQNGGSIIGGTFKAIQERKNAPKAERDPRLPKPQGQTVGSFR
KGLRMLPEAISARLGSKVKLSWKLSGITKLESGGYNLTYETPDGLVSVQSKSVVMTVPSHV
ASGLLRPLSESAANALSKLYYPPVAAVSISYPKEAIRTECLIDGELKGFGQLHPRTQGVETLG
TIYSSSLFPNRAPPGRILLLNYIGGSTNTGILSKSEGELVEAVDRDLRKMLIKPNSTDPLKLGV
RVWPQAIPQFLVGHFDILDTAKSSLTSSGYEGLFLGGNYVAGVALGRCVEGAYETAIEVNNF
MSRYAYK*
Arabidopsis thaliana PPO1 E178Y (SEQ ID NO: 167)
SVAGGPTVGSSKIEGGGGTTITTDCVIVGGGISGLCIAQALATKHPDAAPNLIVTEAKDRVGG
NIITREENGFLWEEGPNSFQPSDPMLTMVVDSGLKDDLVLGDPTAPRFVLWNGKLRPVPSK
LTDLPFFDLMSIGGKIRAGFGALGIRPSPPGREESVEEFVRRNLGDEVFERLIYPFCSGVYA
GDPSKLSMKAAFGKVWKLEQNGGSIIGGTFKAIQERKNAPKAERDPRLPKPQGQTVGSFR
KGLRMLPEAISARLGSKVKLSWKLSGITKLESGGYNLTYETPDGLVSVQSKSVVMTVPSHV
ASGLLRPLSESAANALSKLYYPPVAAVSISYPKEAIRTECLIDGELKGFGQLHPRTQGVETLG
TIYSSSLFPNRAPPGRILLLNYIGGSTNTGILSKSEGELVEAVDRDLRKMLIKPNSTDPLKLGV
RVWPQAIPQFLVGHFDILDTAKSSLTSSGYEGLFLGGNYVAGVALGRCVEGAYETAIEVNNF
MSRYAYK*
Arabidopsis thaliana PPO1 F180Y (SEQ ID NO: 168)
SVAGGPTVGSSKIEGGGGTTITTDCVIVGGGISGLCIAQALATKHPDAAPNLIVTEAKDRVGG
NIITREENGFLWEEGPNSFQPSDPMLTMVVDSGLKDDLVLGDPTAPRFVLWNGKLRPVPSK
LTDLPFFDLMSIGGKIRAGFGALGIRPSPPGREESVEEFVRRNLGDEVFERLIEPYCSGVYA
GDPSKLSMKAAFGKVWKLEQNGGSIIGGTFKAIQERKNAPKAERDPRLPKPQGQTVGSFR
KGLRMLPEAISARLGSKVKLSWKLSGITKLESGGYNLTYETPDGLVSVQSKSVVMTVPSHV
ASGLLRPLSESAANALSKLYYPPVAAVSISYPKEAIRTECLIDGELKGFGQLHPRTQGVETLG
TIYSSSLFPNRAPPGRILLLNYIGGSTNTGILSKSEGELVEAVDRDLRKMLIKPNSTDPLKLGV
RVWPQAIPQFLVGHFDILDTAKSSLTSSGYEGLFLGGNYVAGVALGRCVEGAYETAIEVNNF
MSRYAYK*
Arabidopsis thaliana PPO1 V184K (SEQ ID NO: 169)
SVAGGPTVGSSKIEGGGGTTITTDCVIVGGGISGLCIAQALATKHPDAAPNLIVTEAKDRVGG
NIITREENGFLWEEGPNSFQPSDPMLTMVVDSGLKDDLVLGDPTAPRFVLWNGKLRPVPSK
LTDLPFFDLMSIGGKIRAGFGALGIRPSPPGREESVEEFVRRNLGDEVFERLIEPFCSGKYA
GDPSKLSMKAAFGKVWKLEQNGGSIIGGTFKAIQERKNAPKAERDPRLPKPQGQTVGSFR
KGLRMLPEAISARLGSKVKLSWKLSGITKLESGGYNLTYETPDGLVSVQSKSVVMTVPSHV
ASGLLRPLSESAANALSKLYYPPVAAVSISYPKEAIRTECLIDGELKGFGQLHPRTQGVETLG
TIYSSSLFPNRAPPGRILLLNYIGGSTNTGILSKSEGELVEAVDRDLRKMLIKPNSTDPLKLGV
RVWPQAIPQFLVGHFDILDTAKSSLTSSGYEGLFLGGNYVAGVALGRCVEGAYETAIEVNNF
MSRYAYK*
Arabidopsis thaliana PPO1 Y185S (SEQ ID NO: 170)
SVAGGPTVGSSKIEGGGGTTITTDCVIVGGGISGLCIAQALATKHPDAAPNLIVTEAKDRVGG
NIITREENGFLWEEGPNSFQPSDPMLTMVVDSGLKDDLVLGDPTAPRFVLWNGKLRPVPSK
LTDLPFFDLMSIGGKIRAGFGALGIRPSPPGREESVEEFVRRNLGDEVFERLIEPFCSGVSA
GDPSKLSMKAAFGKVWKLEQNGGSIIGGTFKAIQERKNAPKAERDPRLPKPQGQTVGSFR
KGLRMLPEAISARLGSKVKLSWKLSGITKLESGGYNLTYETPDGLVSVQSKSVVMTVPSHV
ASGLLRPLSESAANALSKLYYPPVAAVSISYPKEAIRTECLIDGELKGFGQLHPRTQGVETLG
TIYSSSLFPNRAPPGRILLLNYIGGSTNTGILSKSEGELVEAVDRDLRKMLIKPNSTDPLKLGV
RVWPQAIPQFLVGHFDILDTAKSSLTSSGYEGLFLGGNYVAGVALGRCVEGAYETAIEVNNF
MSRYAYK*
Arabidopsis thaliana PPO1 A186C (SEQ ID NO: 171)
SVAGGPTVGSSKIEGGGGTTITTDCVIVGGGISGLCIAQALATKHPDAAPNLIVTEAKDRVGG
NIITREENGFLWEEGPNSFQPSDPMLTMVVDSGLKDDLVLGDPTAPRFVLWNGKLRPVPSK
LTDLPFFDLMSIGGKIRAGFGALGIRPSPPGREESVEEFVRRNLGDEVFERLIEPFCSGVYC
GDPSKLSMKAAFGKVWKLEQNGGSIIGGTFKAIQERKNAPKAERDPRLPKPQGQTVGSFR
KGLRMLPEAISARLGSKVKLSWKLSGITKLESGGYNLTYETPDGLVSVQSKSVVMTVPSHV
ASGLLRPLSESAANALSKLYYPPVAAVSISYPKEAIRTECLIDGELKGFGQLHPRTQGVETLG
TIYSSSLFPNRAPPGRILLLNYIGGSTNTGILSKSEGELVEAVDRDLRKMLIKPNSTDPLKLGV
RVWPQAIPQFLVGHFDILDTAKSSLTSSGYEGLFLGGNYVAGVALGRCVEGAYETAIEVNNF
MSRYAYK*
Arabidopsis thaliana PPO1 P237C (SEQ ID NO: 172)
SVAGGPTVGSSKIEGGGGTTITTDCVIVGGGISGLCIAQALATKHPDAAPNLIVTEAKDRVGG
NIITREENGFLWEEGPNSFQPSDPMLTMVVDSGLKDDLVLGDPTAPRFVLWNGKLRPVPSK
LTDLPFFDLMSIGGKIRAGFGALGIRPSPPGREESVEEFVRRNLGDEVFERLIEPFCSGVYA
GDPSKLSMKAAFGKVWKLEQNGGSIIGGTFKAIQERKNAPKAERDPRLPKCQGQTVGSFR
KGLRMLPEAISARLGSKVKLSWKLSGITKLESGGYNLTYETPDGLVSVQSKSVVMTVPSHV
ASGLLRPLSESAANALSKLYYPPVAAVSISYPKEAIRTECLIDGELKGFGQLHPRTQGVETLG
TIYSSSLFPNRAPPGRILLLNYIGGSTNTGILSKSEGELVEAVDRDLRKMLIKPNSTDPLKLGV
RVWPQAIPQFLVGHFDILDTAKSSLTSSGYEGLFLGGNYVAGVALGRCVEGAYETAIEVNNF
MSRYAYK*
Arabidopsis thaliana PPO1 Q238H (SEQ ID NO: 173)
SVAGGPTVGSSKIEGGGGTTITTDCVIVGGGISGLCIAQALATKHPDAAPNLIVTEAKDRVGG
NIITREENGFLWEEGPNSFQPSDPMLTMVVDSGLKDDLVLGDPTAPRFVLWNGKLRPVPSK
LTDLPFFDLMSIGGKIRAGFGALGIRPSPPGREESVEEFVRRNLGDEVFERLIEPFCSGVYA
GDPSKLSMKAAFGKVWKLEQNGGSIIGGTFKAIQERKNAPKAERDPRLPKPHGQTVGSFR
KGLRMLPEAISARLGSKVKLSWKLSGITKLESGGYNLTYETPDGLVSVQSKSVVMTVPSHV
ASGLLRPLSESAANALSKLYYPPVAAVSISYPKEAIRTECLIDGELKGFGQLHPRTQGVETLG
TIYSSSLFPNRAPPGRILLLNYIGGSTNTGILSKSEGELVEAVDRDLRKMLIKPNSTDPLKLGV
RVWPQAIPQFLVGHFDILDTAKSSLTSSGYEGLFLGGNYVAGVALGRCVEGAYETAIEVNNF
MSRYAYK*
Arabidopsis thaliana PPO1 Q238I (SEQ ID NO: 174)
SVAGGPTVGSSKIEGGGGTTITTDCVIVGGGISGLCIAQALATKHPDAAPNLIVTEAKDRVGG
NIITREENGFLWEEGPNSFQPSDPMLTMVVDSGLKDDLVLGDPTAPRFVLWNGKLRPVPSK
LTDLPFFDLMSIGGKIRAGFGALGIRPSPPGREESVEEFVRRNLGDEVFERLIEPFCSGVYA
GDPSKLSMKAAFGKVWKLEQNGGSIIGGTFKAIQERKNAPKAERDPRLPKPIGQTVGSFRK
GLRMLPEAISARLGSKVKLSWKLSGITKLESGGYNLTYETPDGLVSVQSKSVVMTVPSHVA
SGLLRPLSESAANALSKLYYPPVAAVSISYPKEAIRTECLIDGELKGFGQLHPRTQGVETLGT
IYSSSLFPNRAPPGRILLLNYIGGSTNTGILSKSEGELVEAVDRDLRKMLIKPNSTDPLKLGVR
VWPQAIPQFLVGHFDILDTAKSSLTSSGYEGLFLGGNYVAGVALGRCVEGAYETAIEVNNF
MSRYAYK*
Arabidopsis thaliana PPO1 Q238T (SEQ ID NO: 175)
SVAGGPTVGSSKIEGGGGTTITTDCVIVGGGISGLCIAQALATKHPDAAPNLIVTEAKDRVGG
NIITREENGFLWEEGPNSFQPSDPMLTMVVDSGLKDDLVLGDPTAPRFVLWNGKLRPVPSK
LTDLPFFDLMSIGGKIRAGFGALGIRPSPPGREESVEEFVRRNLGDEVFERLIEPFCSGVYA
GDPSKLSMKAAFGKVWKLEQNGGSIIGGTFKAIQERKNAPKAERDPRLPKPTGQTVGSFRK
GLRMLPEAISARLGSKVKLSWKLSGITKLESGGYNLTYETPDGLVSVQSKSVVMTVPSHVA
SGLLRPLSESAANALSKLYYPPVAAVSISYPKEAIRTECLIDGELKGFGQLHPRTQGVETLGT
IYSSSLFPNRAPPGRILLLNYIGGSTNTGILSKSEGELVEAVDRDLRKMLIKPNSTDPLKLGVR
VWPQAIPQFLVGHFDILDTAKSSLTSSGYEGLFLGGNYVAGVALGRCVEGAYETAIEVNNF
MSRYAYK*
Arabidopsis thaliana PPO1 K247C (SEQ ID NO: 176)
SVAGGPTVGSSKIEGGGGTTITTDCVIVGGGISGLCIAQALATKHPDAAPNLIVTEAKDRVGG
NIITREENGFLWEEGPNSFQPSDPMLTMVVDSGLKDDLVLGDPTAPRFVLWNGKLRPVPSK
LTDLPFFDLMSIGGKIRAGFGALGIRPSPPGREESVEEFVRRNLGDEVFERLIEPFCSGVYA
GDPSKLSMKAAFGKVWKLEQNGGSIIGGTFKAIQERKNAPKAERDPRLPKPQGQTVGSFR
CGLRMLPEAISARLGSKVKLSWKLSGITKLESGGYNLTYETPDGLVSVQSKSVVMTVPSHV
ASGLLRPLSESAANALSKLYYPPVAAVSISYPKEAIRTECLIDGELKGFGQLHPRTQGVETLG
TIYSSSLFPNRAPPGRILLLNYIGGSTNTGILSKSEGELVEAVDRDLRKMLIKPNSTDPLKLGV
RVWPQAIPQFLVGHFDILDTAKSSLTSSGYEGLFLGGNYVAGVALGRCVEGAYETAIEVNNF
MSRYAYK*
Arabidopsis thaliana PPO1 K247E (SEQ ID NO: 177)
SVAGGPTVGSSKIEGGGGTTITTDCVIVGGGISGLCIAQALATKHPDAAPNLIVTEAKDRVGG
NIITREENGFLWEEGPNSFQPSDPMLTMVVDSGLKDDLVLGDPTAPRFVLWNGKLRPVPSK
LTDLPFFDLMSIGGKIRAGFGALGIRPSPPGREESVEEFVRRNLGDEVFERLIEPFCSGVYA
GDPSKLSMKAAFGKVWKLEQNGGSIIGGTFKAIQERKNAPKAERDPRLPKPQGQTVGSFR
EGLRMLPEAISARLGSKVKLSWKLSGITKLESGGYNLTYETPDGLVSVQSKSVVMTVPSHV
ASGLLRPLSESAANALSKLYYPPVAAVSISYPKEAIRTECLIDGELKGFGQLHPRTQGVETLG
TIYSSSLFPNRAPPGRILLLNYIGGSTNTGILSKSEGELVEAVDRDLRKMLIKPNSTDPLKLGV
RVWPQAIPQFLVGHFDILDTAKSSLTSSGYEGLFLGGNYVAGVALGRCVEGAYETAIEVNNF
MSRYAYK*
Arabidopsis thaliana PPO1 K247N (SEQ ID NO: 178)
SVAGGPTVGSSKIEGGGGTTITTDCVIVGGGISGLCIAQALATKHPDAAPNLIVTEAKDRVGG
NIITREENGFLWEEGPNSFQPSDPMLTMVVDSGLKDDLVLGDPTAPRFVLWNGKLRPVPSK
LTDLPFFDLMSIGGKIRAGFGALGIRPSPPGREESVEEFVRRNLGDEVFERLIEPFCSGVYA
GDPSKLSMKAAFGKVWKLEQNGGSIIGGTFKAIQERKNAPKAERDPRLPKPQGQTVGSFR
NGLRMLPEAISARLGSKVKLSWKLSGITKLESGGYNLTYETPDGLVSVQSKSVVMTVPSHV
ASGLLRPLSESAANALSKLYYPPVAAVSISYPKEAIRTECLIDGELKGFGQLHPRTQGVETLG
TIYSSSLFPNRAPPGRILLLNYIGGSTNTGILSKSEGELVEAVDRDLRKMLIKPNSTDPLKLGV
RVWPQAIPQFLVGHFDILDTAKSSLTSSGYEGLFLGGNYVAGVALGRCVEGAYETAIEVNNF
MSRYAYK*
Arabidopsis thaliana PPO1 R250G (SEQ ID NO: 179)
SVAGGPTVGSSKIEGGGGTTITTDCVIVGGGISGLCIAQALATKHPDAAPNLIVTEAKDRVGG
NIITREENGFLWEEGPNSFQPSDPMLTMVVDSGLKDDLVLGDPTAPRFVLWNGKLRPVPSK
LTDLPFFDLMSIGGKIRAGFGALGIRPSPPGREESVEEFVRRNLGDEVFERLIEPFCSGVYA
GDPSKLSMKAAFGKVWKLEQNGGSIIGGTFKAIQERKNAPKAERDPRLPKPQGQTVGSFR
KGLGMLPEAISARLGSKVKLSWKLSGITKLESGGYNLTYETPDGLVSVQSKSVVMTVPSHV
ASGLLRPLSESAANALSKLYYPPVAAVSISYPKEAIRTECLIDGELKGFGQLHPRTQGVETLG
TIYSSSLFPNRAPPGRILLLNYIGGSTNTGILSKSEGELVEAVDRDLRKMLIKPNSTDPLKLGV
RVWPQAIPQFLVGHFDILDTAKSSLTSSGYEGLFLGGNYVAGVALGRCVEGAYETAIEVNNF
MSRYAYK*
Arabidopsis thaliana PPO1 S271L (SEQ ID NO: 180)
SVAGGPTVGSSKIEGGGGTTITTDCVIVGGGISGLCIAQALATKHPDAAPNLIVTEAKDRVGG
NIITREENGFLWEEGPNSFQPSDPMLTMVVDSGLKDDLVLGDPTAPRFVLWNGKLRPVPSK
LTDLPFFDLMSIGGKIRAGFGALGIRPSPPGREESVEEFVRRNLGDEVFERLIEPFCSGVYA
GDPSKLSMKAAFGKVWKLEQNGGSIIGGTFKAIQERKNAPKAERDPRLPKPQGQTVGSFR
KGLRMLPEAISARLGSKVKLSWKLLGITKLESGGYNLTYETPDGLVSVQSKSVVMTVPSHVA
SGLLRPLSESAANALSKLYYPPVAAVSISYPKEAIRTECLIDGELKGFGQLHPRTQGVETLGT
IYSSSLFPNRAPPGRILLLNYIGGSTNTGILSKSEGELVEAVDRDLRKMLIKPNSTDPLKLGVR
VWPQAIPQFLVGHFDILDTAKSSLTSSGYEGLFLGGNYVAGVALGRCVEGAYETAIEVNNF
MSRYAYK*
Arabidopsis thaliana PPO1 L326C (SEQ ID NO: 181)
SVAGGPTVGSSKIEGGGGTTITTDCVIVGGGISGLCIAQALATKHPDAAPNLIVTEAKDRVGG
NIITREENGFLWEEGPNSFQPSDPMLTMVVDSGLKDDLVLGDPTAPRFVLWNGKLRPVPSK
LTDLPFFDLMSIGGKIRAGFGALGIRPSPPGREESVEEFVRRNLGDEVFERLIEPFCSGVYA
GDPSKLSMKAAFGKVWKLEQNGGSIIGGTFKAIQERKNAPKAERDPRLPKPQGQTVGSFR
KGLRMLPEAISARLGSKVKLSWKLSGITKLESGGYNLTYETPDGLVSVQSKSVVMTVPSHV
ASGLLRPLSESAANALSKCYYPPVAAVSISYPKEAIRTECLIDGELKGFGQLHPRTQGVETL
GTIYSSSLFPNRAPPGRILLLNYIGGSTNTGILSKSEGELVEAVDRDLRKMLIKPNSTDPLKLG
VRVWPQAIPQFLVGHFDILDTAKSSLTSSGYEGLFLGGNYVAGVALGRCVEGAYETAIEVN
NFMSRYAYK*
Arabidopsis thaliana PPO1 Y327D (SEQ ID NO: 182)
SVAGGPTVGSSKIEGGGGTTITTDCVIVGGGISGLCIAQALATKHPDAAPNLIVTEAKDRVGG
NIITREENGFLWEEGPNSFQPSDPMLTMVVDSGLKDDLVLGDPTAPRFVLWNGKLRPVPSK
LTDLPFFDLMSIGGKIRAGFGALGIRPSPPGREESVEEFVRRNLGDEVFERLIEPFCSGVYA
GDPSKLSMKAAFGKVWKLEQNGGSIIGGTFKAIQERKNAPKAERDPRLPKPQGQTVGSFR
KGLRMLPEAISARLGSKVKLSWKLSGITKLESGGYNLTYETPDGLVSVQSKSVVMTVPSHV
ASGLLRPLSESAANALSKLDYPPVAAVSISYPKEAIRTECLIDGELKGFGQLHPRTQGVETLG
TIYSSSLFPNRAPPGRILLLNYIGGSTNTGILSKSEGELVEAVDRDLRKMLIKPNSTDPLKLGV
RVWPQAIPQFLVGHFDILDTAKSSLTSSGYEGLFLGGNYVAGVALGRCVEGAYETAIEVNNF
MSRYAYK*
Arabidopsis thaliana PPO1 Y327F (SEQ ID NO: 183)
SVAGGPTVGSSKIEGGGGTTITTDCVIVGGGISGLCIAQALATKHPDAAPNLIVTEAKDRVGG
NIITREENGFLWEEGPNSFQPSDPMLTMVVDSGLKDDLVLGDPTAPRFVLWNGKLRPVPSK
LTDLPFFDLMSIGGKIRAGFGALGIRPSPPGREESVEEFVRRNLGDEVFERLIEPFCSGVYA
GDPSKLSMKAAFGKVWKLEQNGGSIIGGTFKAIQERKNAPKAERDPRLPKPQGQTVGSFR
KGLRMLPEAISARLGSKVKLSWKLSGITKLESGGYNLTYETPDGLVSVQSKSVVMTVPSHV
ASGLLRPLSESAANALSKLFYPPVAAVSISYPKEAIRTECLIDGELKGFGQLHPRTQGVETLG
TIYSSSLFPNRAPPGRILLLNYIGGSTNTGILSKSEGELVEAVDRDLRKMLIKPNSTDPLKLGV
RVWPQAIPQFLVGHFDILDTAKSSLTSSGYEGLFLGGNYVAGVALGRCVEGAYETAIEVNNF
MSRYAYK*
Arabidopsis thaliana PPO1 Y327P (SEQ ID NO: 184)
SVAGGPTVGSSKIEGGGGTTITTDCVIVGGGISGLCIAQALATKHPDAAPNLIVTEAKDRVGG
NIITREENGFLWEEGPNSFQPSDPMLTMVVDSGLKDDLVLGDPTAPRFVLWNGKLRPVPSK
LTDLPFFDLMSIGGKIRAGFGALGIRPSPPGREESVEEFVRRNLGDEVFERLIEPFCSGVYA
GDPSKLSMKAAFGKVWKLEQNGGSIIGGTFKAIQERKNAPKAERDPRLPKPQGQTVGSFR
KGLRMLPEAISARLGSKVKLSWKLSGITKLESGGYNLTYETPDGLVSVQSKSVVMTVPSHV
ASGLLRPLSESAANALSKLPYPPVAAVSISYPKEAIRTECLIDGELKGFGQLHPRTQGVETLG
TIYSSSLFPNRAPPGRILLLNYIGGSTNTGILSKSEGELVEAVDRDLRKMLIKPNSTDPLKLGV
RVWPQAIPQFLVGHFDILDTAKSSLTSSGYEGLFLGGNYVAGVALGRCVEGAYETAIEVNNF
MSRYAYK*
Arabidopsis thaliana PPO1 Y327Q (SEQ ID NO: 185)
SVAGGPTVGSSKIEGGGGTTITTDCVIVGGGISGLCIAQALATKHPDAAPNLIVTEAKDRVGG
NIITREENGFLWEEGPNSFQPSDPMLTMVVDSGLKDDLVLGDPTAPRFVLWNGKLRPVPSK
LTDLPFFDLMSIGGKIRAGFGALGIRPSPPGREESVEEFVRRNLGDEVFERLIEPFCSGVYA
GDPSKLSMKAAFGKVWKLEQNGGSIIGGTFKAIQERKNAPKAERDPRLPKPQGQTVGSFR
KGLRMLPEAISARLGSKVKLSWKLSGITKLESGGYNLTYETPDGLVSVQSKSVVMTVPSHV
ASGLLRPLSESAANALSKLQYPPVAAVSISYPKEAIRTECLIDGELKGFGQLHPRTQGVETLG
TIYSSSLFPNRAPPGRILLLNYIGGSTNTGILSKSEGELVEAVDRDLRKMLIKPNSTDPLKLGV
RVWPQAIPQFLVGHFDILDTAKSSLTSSGYEGLFLGGNYVAGVALGRCVEGAYETAIEVNNF
MSRYAYK*
Arabidopsis thaliana PPO1 Y327R (SEQ ID NO: 186)
SVAGGPTVGSSKIEGGGGTTITTDCVIVGGGISGLCIAQALATKHPDAAPNLIVTEAKDRVGG
NIITREENGFLWEEGPNSFQPSDPMLTMVVDSGLKDDLVLGDPTAPRFVLWNGKLRPVPSK
LTDLPFFDLMSIGGKIRAGFGALGIRPSPPGREESVEEFVRRNLGDEVFERLIEPFCSGVYA
GDPSKLSMKAAFGKVWKLEQNGGSIIGGTFKAIQERKNAPKAERDPRLPKPQGQTVGSFR
KGLRMLPEAISARLGSKVKLSWKLSGITKLESGGYNLTYETPDGLVSVQSKSVVMTVPSHV
ASGLLRPLSESAANALSKLYYRPVAAVSISYPKEAIRTECLIDGELKGFGQLHPRTQGVETLG
TIYSSSLFPNRAPPGRILLLNYIGGSTNTGILSKSEGELVEAVDRDLRKMLIKPNSTDPLKLGV
RVWPQAIPQFLVGHFDILDTAKSSLTSSGYEGLFLGGNYVAGVALGRCVEGAYETAIEVNNF
MSRYAYK*
Arabidopsis thaliana PPO1 Y327T (SEQ ID NO: 187)
SVAGGPTVGSSKIEGGGGTTITTDCVIVGGGISGLCIAQALATKHPDAAPNLIVTEAKDRVGG
NIITREENGFLWEEGPNSFQPSDPMLTMVVDSGLKDDLVLGDPTAPRFVLWNGKLRPVPSK
LTDLPFFDLMSIGGKIRAGFGALGIRPSPPGREESVEEFVRRNLGDEVFERLIEPFCSGVYA
GDPSKLSMKAAFGKVWKLEQNGGSIIGGTFKAIQERKNAPKAERDPRLPKPQGQTVGSFR
KGLRMLPEAISARLGSKVKLSWKLSGITKLESGGYNLTYETPDGLVSVQSKSVVMTVPSHV
ASGLLRPLSESAANALSKLTYPPVAAVSISYPKEAIRTECLIDGELKGFGQLHPRTQGVETLG
TIYSSSLFPNRAPPGRILLLNYIGGSTNTGILSKSEGELVEAVDRDLRKMLIKPNSTDPLKLGV
RVWPQAIPQFLVGHFDILDTAKSSLTSSGYEGLFLGGNYVAGVALGRCVEGAYETAIEVNNF
MSRYAYK*
Arabidopsis thaliana PPO1 Y328F (SEQ ID NO: 188)
SVAGGPTVGSSKIEGGGGTTITTDCVIVGGGISGLCIAQALATKHPDAAPNLIVTEAKDRVGG
NIITREENGFLWEEGPNSFQPSDPMLTMVVDSGLKDDLVLGDPTAPRFVLWNGKLRPVPSK
LTDLPFFDLMSIGGKIRAGFGALGIRPSPPGREESVEEFVRRNLGDEVFERLIEPFCSGVYA
GDPSKLSMKAAFGKVWKLEQNGGSIIGGTFKAIQERKNAPKAERDPRLPKPQGQTVGSFR
KGLRMLPEAISARLGSKVKLSWKLSGITKLESGGYNLTYETPDGLVSVQSKSVVMTVPSHV
ASGLLRPLSESAANALSKLYFPPVAAVSISYPKEAIRTECLIDGELKGFGQLHPRTQGVETLG
TIYSSSLFPNRAPPGRILLLNYIGGSTNTGILSKSEGELVEAVDRDLRKMLIKPNSTDPLKLGV
RVWPQAIPQFLVGHFDILDTAKSSLTSSGYEGLFLGGNYVAGVALGRCVEGAYETAIEVNNF
MSRYAYK*
Arabidopsis thaliana PPO1 Y331L (SEQ ID NO: 189)
SVAGGPTVGSSKIEGGGGTTITTDCVIVGGGISGLCIAQALATKHPDAAPNLIVTEAKDRVGG
NIITREENGFLWEEGPNSFQPSDPMLTMVVDSGLKDDLVLGDPTAPRFVLWNGKLRPVPSK
LTDLPFFDLMSIGGKIRAGFGALGIRPSPPGREESVEEFVRRNLGDEVFERLIEPFCSGVYA
GDPSKLSMKAAFGKVWKLEQNGGSIIGGTFKAIQERKNAPKAERDPRLPKPQGQTVGSFR
KGLRMLPEAISARLGSKVKLSWKLSGITKLESGGYNLTYETPDGLVSVQSKSVVMTVPSHV
ASGLLRPLSESAANALSKLYYPPLAAVSISYPKEAIRTECLIDGELKGFGQLHPRTQGVETLG
TIYSSSLFPNRAPPGRILLLNYIGGSTNTGILSKSEGELVEAVDRDLRKMLIKPNSTDPLKLGV
RVWPQAIPQFLVGHFDILDTAKSSLTSSGYEGLFLGGNYVAGVALGRCVEGAYETAIEVNNF
MSRYAYK*
Arabidopsis thaliana PPO1 Y331M (SEQ ID NO: 190)
SVAGGPTVGSSKIEGGGGTTITTDCVIVGGGISGLCIAQALATKHPDAAPNLIVTEAKDRVGG
NIITREENGFLWEEGPNSFQPSDPMLTMVVDSGLKDDLVLGDPTAPRFVLWNGKLRPVPSK
LTDLPFFDLMSIGGKIRAGFGALGIRPSPPGREESVEEFVRRNLGDEVFERLIEPFCSGVYA
GDPSKLSMKAAFGKVWKLEQNGGSIIGGTFKAIQERKNAPKAERDPRLPKPQGQTVGSFR
KGLRMLPEAISARLGSKVKLSWKLSGITKLESGGYNLTYETPDGLVSVQSKSVVMTVPSHV
ASGLLRPLSESAANALSKLYYPPMAAVSISYPKEAIRTECLIDGELKGFGQLHPRTQGVETL
GTIYSSSLFPNRAPPGRILLLNYIGGSTNTGILSKSEGELVEAVDRDLRKMLIKPNSTDPLKLG
VRVWPQAIPQFLVGHFDILDTAKSSLTSSGYEGLFLGGNYVAGVALGRCVEGAYETAIEVN
NFMSRYAYK*
Arabidopsis thaliana PPO1 G370A (SEQ ID NO: 191)
SVAGGPTVGSSKIEGGGGTTITTDCVIVGGGISGLCIAQALATKHPDAAPNLIVTEAKDRVGG
NIITREENGFLWEEGPNSFQPSDPMLTMVVDSGLKDDLVLGDPTAPRFVLWNGKLRPVPSK
LTDLPFFDLMSIGGKIRAGFGALGIRPSPPGREESVEEFVRRNLGDEVFERLIEPFCSGVYA
GDPSKLSMKAAFGKVWKLEQNGGSIIGGTFKAIQERKNAPKAERDPRLPKPQGQTVGSFR
KGLRMLPEAISARLGSKVKLSWKLSGITKLESGGYNLTYETPDGLVSVQSKSVVMTVPSHV
ASGLLRPLSESAANALSKLYYPPVAAVSISYPKEAIRTECLIDGELKGFGQLHPRTQGVETLA
TIYSSSLFPNRAPPGRILLLNYIGGSTNTGILSKSEGELVEAVDRDLRKMLIKPNSTDPLKLGV
RVWPQAIPQFLVGHFDILDTAKSSLTSSGYEGLFLGGNYVAGVALGRCVEGAYETAIEVNNF
MSRYAYK*
Arabidopsis thaliana PPO1 P379G (SEQ ID NO: 192)
SVAGGPTVGSSKIEGGGGTTITTDCVIVGGGISGLCIAQALATKHPDAAPNLIVTEAKDRVGG
NIITREENGFLWEEGPNSFQPSDPMLTMVVDSGLKDDLVLGDPTAPRFVLWNGKLRPVPSK
LTDLPFFDLMSIGGKIRAGFGALGIRPSPPGREESVEEFVRRNLGDEVFERLIEPFCSGVYA
GDPSKLSMKAAFGKVWKLEQNGGSIIGGTFKAIQERKNAPKAERDPRLPKPQGQTVGSFR
KGLRMLPEAISARLGSKVKLSWKLSGITKLESGGYNLTYETPDGLVSVQSKSVVMTVPSHV
ASGLLRPLSESAANALSKLYYPPVAAVSISYPKEAIRTECLIDGELKGFGQLHPRTQGVETLG
TIYSSSLFGNRAPPGRILLLNYIGGSTNTGILSKSEGELVEAVDRDLRKMLIKPNSTDPLKLGV
RVWPQAIPQFLVGHFDILDTAKSSLTSSGYEGLFLGGNYVAGVALGRCVEGAYETAIEVNNF
MSRYAYK*
Arabidopsis thaliana PPO1 P384M (SEQ ID NO: 193)
SVAGGPTVGSSKIEGGGGTTITTDCVIVGGGISGLCIAQALATKHPDAAPNLIVTEAKDRVGG
NIITREENGFLWEEGPNSFQPSDPMLTMVVDSGLKDDLVLGDPTAPRFVLWNGKLRPVPSK
LTDLPFFDLMSIGGKIRAGFGALGIRPSPPGREESVEEFVRRNLGDEVFERLIEPFCSGVYA
GDPSKLSMKAAFGKVWKLEQNGGSIIGGTFKAIQERKNAPKAERDPRLPKPQGQTVGSFR
KGLRMLPEAISARLGSKVKLSWKLSGITKLESGGYNLTYETPDGLVSVQSKSVVMTVPSHV
ASGLLRPLSESAANALSKLYYPPVAAVSISYPKEAIRTECLIDGELKGFGQLHPRTQGVETLG
TIYSSSLFPNRAPMGRILLLNYIGGSTNTGILSKSEGELVEAVDRDLRKMLIKPNSTDPLKLGV
RVWPQAIPQFLVGHFDILDTAKSSLTSSGYEGLFLGGNYVAGVALGRCVEGAYETAIEVNNF
MSRYAYK*
Arabidopsis thaliana PPO1 L390V (SEQ ID NO: 194)
SVAGGPTVGSSKIEGGGGTTITTDCVIVGGGISGLCIAQALATKHPDAAPNLIVTEAKDRVGG
NIITREENGFLWEEGPNSFQPSDPMLTMVVDSGLKDDLVLGDPTAPRFVLWNGKLRPVPSK
LTDLPFFDLMSIGGKIRAGFGALGIRPSPPGREESVEEFVRRNLGDEVFERLIEPFCSGVYA
GDPSKLSMKAAFGKVWKLEQNGGSIIGGTFKAIQERKNAPKAERDPRLPKPQGQTVGSFR
KGLRMLPEAISARLGSKVKLSWKLSGITKLESGGYNLTYETPDGLVSVQSKSVVMTVPSHV
ASGLLRPLSESAANALSKLYYPPVAAVSISYPKEAIRTECLIDGELKGFGQLHPRTQGVETLG
TIYSSSLFPNRAPPGRILLVNYIGGSTNTGILSKSEGELVEAVDRDLRKMLIKPNSTDPLKLGV
RVWPQAIPQFLVGHFDILDTAKSSLTSSGYEGLFLGGNYVAGVALGRCVEGAYETAIEVNNF
MSRYAYK*
Arabidopsis thaliana PPO1 Y392C (SEQ ID NO: 195)
SVAGGPTVGSSKIEGGGGTTITTDCVIVGGGISGLCIAQALATKHPDAAPNLIVTEAKDRVGG
NIITREENGFLWEEGPNSFQPSDPMLTMVVDSGLKDDLVLGDPTAPRFVLWNGKLRPVPSK
LTDLPFFDLMSIGGKIRAGFGALGIRPSPPGREESVEEFVRRNLGDEVFERLIEPFCSGVYA
GDPSKLSMKAAFGKVWKLEQNGGSIIGGTFKAIQERKNAPKAERDPRLPKPQGQTVGSFR
KGLRMLPEAISARLGSKVKLSWKLSGITKLESGGYNLTYETPDGLVSVQSKSVVMTVPSHV
ASGLLRPLSESAANALSKLYYPPVAAVSISYPKEAIRTECLIDGELKGFGQLHPRTQGVETLG
TIYSSSLFPNRAPPGRILLLNCIGGSTNTGILSKSEGELVEAVDRDLRKMLIKPNSTDPLKLGV
RVWPQAIPQFLVGHFDILDTAKSSLTSSGYEGLFLGGNYVAGVALGRCVEGAYETAIEVNNF
MSRYAYK*
Arabidopsis thaliana PPO1 Y392L (SEQ ID NO: 196)
SVAGGPTVGSSKIEGGGGTTITTDCVIVGGGISGLCIAQALATKHPDAAPNLIVTEAKDRVGG
NIITREENGFLWEEGPNSFQPSDPMLTMVVDSGLKDDLVLGDPTAPRFVLWNGKLRPVPSK
LTDLPFFDLMSIGGKIRAGFGALGIRPSPPGREESVEEFVRRNLGDEVFERLIEPFCSGVYA
GDPSKLSMKAAFGKVWKLEQNGGSIIGGTFKAIQERKNAPKAERDPRLPKPQGQTVGSFR
KGLRMLPEAISARLGSKVKLSWKLSGITKLESGGYNLTYETPDGLVSVQSKSVVMTVPSHV
ASGLLRPLSESAANALSKLYYPPVAAVSISYPKEAIRTECLIDGELKGFGQLHPRTQGVETLG
TIYSSSLFPNRAPPGRILLLNLIGGSTNTGILSKSEGELVEAVDRDLRKMLIKPNSTDPLKLGV
RVWPQAIPQFLVGHFDILDTAKSSLTSSGYEGLFLGGNYVAGVALGRCVEGAYETAIEVNNF
MSRYAYK*
Arabidopsis thaliana PPO1 Y392M (SEQ ID NO: 197)
SVAGGPTVGSSKIEGGGGTTITTDCVIVGGGISGLCIAQALATKHPDAAPNLIVTEAKDRVGG
NIITREENGFLWEEGPNSFQPSDPMLTMVVDSGLKDDLVLGDPTAPRFVLWNGKLRPVPSK
LTDLPFFDLMSIGGKIRAGFGALGIRPSPPGREESVEEFVRRNLGDEVFERLIEPFCSGVYA
GDPSKLSMKAAFGKVWKLEQNGGSIIGGTFKAIQERKNAPKAERDPRLPKPQGQTVGSFR
KGLRMLPEAISARLGSKVKLSWKLSGITKLESGGYNLTYETPDGLVSVQSKSVVMTVPSHV
ASGLLRPLSESAANALSKLYYPPVAAVSISYPKEAIRTECLIDGELKGFGQLHPRTQGVETLG
TIYSSSLFPNRAPPGRILLLNMIGGSTNTGILSKSEGELVEAVDRDLRKMLIKPNSTDPLKLGV
RVWPQAIPQFLVGHFDILDTAKSSLTSSGYEGLFLGGNYVAGVALGRCVEGAYETAIEVNNF
MSRYAYK*
Arabidopsis thaliana PPO1 Y392T (SEQ ID NO: 198)
SVAGGPTVGSSKIEGGGGTTITTDCVIVGGGISGLCIAQALATKHPDAAPNLIVTEAKDRVGG
NIITREENGFLWEEGPNSFQPSDPMLTMVVDSGLKDDLVLGDPTAPRFVLWNGKLRPVPSK
LTDLPFFDLMSIGGKIRAGFGALGIRPSPPGREESVEEFVRRNLGDEVFERLIEPFCSGVYA
GDPSKLSMKAAFGKVWKLEQNGGSIIGGTFKAIQERKNAPKAERDPRLPKPQGQTVGSFR
KGLRMLPEAISARLGSKVKLSWKLSGITKLESGGYNLTYETPDGLVSVQSKSVVMTVPSHV
ASGLLRPLSESAANALSKLYYPPVAAVSISYPKEAIRTECLIDGELKGFGQLHPRTQGVETLG
TIYSSSLFPNRAPPGRILLLNTIGGSTNTGILSKSEGELVEAVDRDLRKMLIKPNSTDPLKLGV
RVWPQAIPQFLVGHFDILDTAKSSLTSSGYEGLFLGGNYVAGVALGRCVEGAYETAIEVNNF
MSRYAYK*
Arabidopsis thaliana PPO1 Y392V (SEQ ID NO: 199)
SVAGGPTVGSSKIEGGGGTTITTDCVIVGGGISGLCIAQALATKHPDAAPNLIVTEAKDRVGG
NIITREENGFLWEEGPNSFQPSDPMLTMVVDSGLKDDLVLGDPTAPRFVLWNGKLRPVPSK
LTDLPFFDLMSIGGKIRAGFGALGIRPSPPGREESVEEFVRRNLGDEVFERLIEPFCSGVYA
GDPSKLSMKAAFGKVWKLEQNGGSIIGGTFKAIQERKNAPKAERDPRLPKPQGQTVGSFR
KGLRMLPEAISARLGSKVKLSWKLSGITKLESGGYNLTYETPDGLVSVQSKSVVMTVPSHV
ASGLLRPLSESAANALSKLYYPPVAAVSISYPKEAIRTECLIDGELKGFGQLHPRTQGVETLG
TIYSSSLFPNRAPPGRILLLNVIGGSTNTGILSKSEGELVEAVDRDLRKMLIKPNSTDPLKLGV
RVWPQAIPQFLVGHFDILDTAKSSLTSSGYEGLFLGGNYVAGVALGRCVEGAYETAIEVNNF
MSRYAYK*
Arabidopsis thaliana PPO1 T397R (SEQ ID NO: 200)
SVAGGPTVGSSKIEGGGGTTITTDCVIVGGGISGLCIAQALATKHPDAAPNLIVTEAKDRVGG
NIITREENGFLWEEGPNSFQPSDPMLTMVVDSGLKDDLVLGDPTAPRFVLWNGKLRPVPSK
LTDLPFFDLMSIGGKIRAGFGALGIRPSPPGREESVEEFVRRNLGDEVFERLIEPFCSGVYA
GDPSKLSMKAAFGKVWKLEQNGGSIIGGTFKAIQERKNAPKAERDPRLPKPQGQTVGSFR
KGLRMLPEAISARLGSKVKLSWKLSGITKLESGGYNLTYETPDGLVSVQSKSVVMTVPSHV
ASGLLRPLSESAANALSKLYYPPVAAVSISYPKEAIRTECLIDGELKGFGQLHPRTQGVETLG
TIYSSSLFPNRAPPGRILLLNYIGGSRNTGILSKSEGELVEAVDRDLRKMLIKPNSTDPLKLGV
RVWPQAIPQFLVGHFDILDTAKSSLTSSGYEGLFLGGNYVAGVALGRCVEGAYETAIEVNNF
MSRYAYK*
Arabidopsis thaliana PPO1 V413C (SEQ ID NO: 201)
SVAGGPTVGSSKIEGGGGTTITTDCVIVGGGISGLCIAQALATKHPDAAPNLIVTEAKDRVGG
NIITREENGFLWEEGPNSFQPSDPMLTMVVDSGLKDDLVLGDPTAPRFVLWNGKLRPVPSK
LTDLPFFDLMSIGGKIRAGFGALGIRPSPPGREESVEEFVRRNLGDEVFERLIEPFCSGVYA
GDPSKLSMKAAFGKVWKLEQNGGSIIGGTFKAIQERKNAPKAERDPRLPKPQGQTVGSFR
KGLRMLPEAISARLGSKVKLSWKLSGITKLESGGYNLTYETPDGLVSVQSKSVVMTVPSHV
ASGLLRPLSESAANALSKLYYPPVAAVSISYPKEAIRTECLIDGELKGFGQLHPRTQGVETLG
TIYSSSLFPNRAPPGRILLLNYIGGSTNTGILSKSEGELVEACDRDLRKMLIKPNSTDPLKLGV
RVWPQAIPQFLVGHFDILDTAKSSLTSSGYEGLFLGGNYVAGVALGRCVEGAYETAIEVNNF
MSRYAYK*
Arabidopsis thaliana PPO1 V413I (SEQ ID NO: 202)
SVAGGPTVGSSKIEGGGGTTITTDCVIVGGGISGLCIAQALATKHPDAAPNLIVTEAKDRVGG
NIITREENGFLWEEGPNSFQPSDPMLTMVVDSGLKDDLVLGDPTAPRFVLWNGKLRPVPSK
LTDLPFFDLMSIGGKIRAGFGALGIRPSPPGREESVEEFVRRNLGDEVFERLIEPFCSGVYA
GDPSKLSMKAAFGKVWKLEQNGGSIIGGTFKAIQERKNAPKAERDPRLPKPQGQTVGSFR
KGLRMLPEAISARLGSKVKLSWKLSGITKLESGGYNLTYETPDGLVSVQSKSVVMTVPSHV
ASGLLRPLSESAANALSKLYYPPVAAVSISYPKEAIRTECLIDGELKGFGQLHPRTQGVETLG
TIYSSSLFPNRAPPGRILLLNYIGGSTNTGILSKSEGELVEAIDRDLRKMLIKPNSTDPLKLGV
RVWPQAIPQFLVGHFDILDTAKSSLTSSGYEGLFLGGNYVAGVALGRCVEGAYETAIEVNNF
MSRYAYK*
Arabidopsis thaliana PPO1 D414H (SEQ ID NO: 203)
SVAGGPTVGSSKIEGGGGTTITTDCVIVGGGISGLCIAQALATKHPDAAPNLIVTEAKDRVGG
NIITREENGFLWEEGPNSFQPSDPMLTMVVDSGLKDDLVLGDPTAPRFVLWNGKLRPVPSK
LTDLPFFDLMSIGGKIRAGFGALGIRPSPPGREESVEEFVRRNLGDEVFERLIEPFCSGVYA
GDPSKLSMKAAFGKVWKLEQNGGSIIGGTFKAIQERKNAPKAERDPRLPKPQGQTVGSFR
KGLRMLPEAISARLGSKVKLSWKLSGITKLESGGYNLTYETPDGLVSVQSKSVVMTVPSHV
ASGLLRPLSESAANALSKLYYPPVAAVSISYPKEAIRTECLIDGELKGFGQLHPRTQGVETLG
TIYSSSLFPNRAPPGRILLLNYIGGSTNTGILSKSEGELVEAVHRDLRKMLIKPNSTDPLKLGV
RVWPQAIPQFLVGHFDILDTAKSSLTSSGYEGLFLGGNYVAGVALGRCVEGAYETAIEVNNF
MSRYAYK*
Arabidopsis thaliana PPO1 D414L (SEQ ID NO: 204)
SVAGGPTVGSSKIEGGGGTTITTDCVIVGGGISGLCIAQALATKHPDAAPNLIVTEAKDRVGG
NIITREENGFLWEEGPNSFQPSDPMLTMVVDSGLKDDLVLGDPTAPRFVLWNGKLRPVPSK
LTDLPFFDLMSIGGKIRAGFGALGIRPSPPGREESVEEFVRRNLGDEVFERLIEPFCSGVYA
GDPSKLSMKAAFGKVWKLEQNGGSIIGGTFKAIQERKNAPKAERDPRLPKPQGQTVGSFR
KGLRMLPEAISARLGSKVKLSWKLSGITKLESGGYNLTYETPDGLVSVQSKSVVMTVPSHV
ASGLLRPLSESAANALSKLYYPPVAAVSISYPKEAIRTECLIDGELKGFGQLHPRTQGVETLG
TIYSSSLFPNRAPPGRILLLNYIGGSTNTGILSKSEGELVEAVLRDLRKMLIKPNSTDPLKLGV
RVWPQAIPQFLVGHFDILDTAKSSLTSSGYEGLFLGGNYVAGVALGRCVEGAYETAIEVNNF
MSRYAYK*
Arabidopsis thaliana PPO1 D414N (SEQ ID NO: 205)
SVAGGPTVGSSKIEGGGGTTITTDCVIVGGGISGLCIAQALATKHPDAAPNLIVTEAKDRVGG
NIITREENGFLWEEGPNSFQPSDPMLTMVVDSGLKDDLVLGDPTAPRFVLWNGKLRPVPSK
LTDLPFFDLMSIGGKIRAGFGALGIRPSPPGREESVEEFVRRNLGDEVFERLIEPFCSGVYA
GDPSKLSMKAAFGKVWKLEQNGGSIIGGTFKAIQERKNAPKAERDPRLPKPQGQTVGSFR
KGLRMLPEAISARLGSKVKLSWKLSGITKLESGGYNLTYETPDGLVSVQSKSVVMTVPSHV
ASGLLRPLSESAANALSKLYYPPVAAVSISYPKEAIRTECLIDGELKGFGQLHPRTQGVETLG
TIYSSSLFPNRAPPGRILLLNYIGGSTNTGILSKSEGELVEAVNRDLRKMLIKPNSTDPLKLGV
RVWPQAIPQFLVGHFDILDTAKSSLTSSGYEGLFLGGNYVAGVALGRCVEGAYETAIEVNNF
MSRYAYK*
Arabidopsis thaliana PPO1 R415K (SEQ ID NO: 206)
SVAGGPTVGSSKIEGGGGTTITTDCVIVGGGISGLCIAQALATKHPDAAPNLIVTEAKDRVGG
NIITREENGFLWEEGPNSFQPSDPMLTMVVDSGLKDDLVLGDPTAPRFVLWNGKLRPVPSK
LTDLPFFDLMSIGGKIRAGFGALGIRPSPPGREESVEEFVRRNLGDEVFERLIEPFCSGVYA
GDPSKLSMKAAFGKVWKLEQNGGSIIGGTFKAIQERKNAPKAERDPRLPKPQGQTVGSFR
KGLRMLPEAISARLGSKVKLSWKLSGITKLESGGYNLTYETPDGLVSVQSKSVVMTVPSHV
ASGLLRPLSESAANALSKLYYPPVAAVSISYPKEAIRTECLIDGELKGFGQLHPRTQGVETLG
TIYSSSLFPNRAPPGRILLLNYIGGSTNTGILSKSEGELVEAVDKDLRKMLIKPNSTDPLKLGV
RVWPQAIPQFLVGHFDILDTAKSSLTSSGYEGLFLGGNYVAGVALGRCVEGAYETAIEVNNF
MSRYAYK*
Arabidopsis thaliana PPO1 T427Q (SEQ ID NO: 207)
SVAGGPTVGSSKIEGGGGTTITTDCVIVGGGISGLCIAQALATKHPDAAPNLIVTEAKDRVGG
NIITREENGFLWEEGPNSFQPSDPMLTMVVDSGLKDDLVLGDPTAPRFVLWNGKLRPVPSK
LTDLPFFDLMSIGGKIRAGFGALGIRPSPPGREESVEEFVRRNLGDEVFERLIEPFCSGVYA
GDPSKLSMKAAFGKVWKLEQNGGSIIGGTFKAIQERKNAPKAERDPRLPKPQGQTVGSFR
KGLRMLPEAISARLGSKVKLSWKLSGITKLESGGYNLTYETPDGLVSVQSKSVVMTVPSHV
ASGLLRPLSESAANALSKLYYPPVAAVSISYPKEAIRTECLIDGELKGFGQLHPRTQGVETLG
TIYSSSLFPNRAPPGRILLLNYIGGSTNTGILSKSEGELVEAVDRDLRKMLIKPNSQDPLKLGV
RVWPQAIPQFLVGHFDILDTAKSSLTSSGYEGLFLGGNYVAGVALGRCVEGAYETAIEVNNF
MSRYAYK*
Arabidopsis thaliana PPO1 F444R (SEQ ID NO: 208)
SVAGGPTVGSSKIEGGGGTTITTDCVIVGGGISGLCIAQALATKHPDAAPNLIVTEAKDRVGG
NIITREENGFLWEEGPNSFQPSDPMLTMVVDSGLKDDLVLGDPTAPRFVLWNGKLRPVPSK
LTDLPFFDLMSIGGKIRAGFGALGIRPSPPGREESVEEFVRRNLGDEVFERLIEPFCSGVYA
GDPSKLSMKAAFGKVWKLEQNGGSIIGGTFKAIQERKNAPKAERDPRLPKPQGQTVGSFR
KGLRMLPEAISARLGSKVKLSWKLSGITKLESGGYNLTYETPDGLVSVQSKSVVMTVPSHV
ASGLLRPLSESAANALSKLYYPPVAAVSISYPKEAIRTECLIDGELKGFGQLHPRTQGVETLG
TIYSSSLFPNRAPPGRILLLNYIGGSTNTGILSKSEGELVEAVDRDLRKMLIKPNSTDPLKLGV
RVWPQAIPQRLVGHFDILDTAKSSLTSSGYEGLFLGGNYVAGVALGRCVEGAYETAIEVNN
FMSRYAYK*
Arabidopsis thaliana PPO1 L445M (SEQ ID NO: 209)
SVAGGPTVGSSKIEGGGGTTITTDCVIVGGGISGLCIAQALATKHPDAAPNLIVTEAKDRVGG
NIITREENGFLWEEGPNSFQPSDPMLTMVVDSGLKDDLVLGDPTAPRFVLWNGKLRPVPSK
LTDLPFFDLMSIGGKIRAGFGALGIRPSPPGREESVEEFVRRNLGDEVFERLIEPFCSGVYA
GDPSKLSMKAAFGKVWKLEQNGGSIIGGTFKAIQERKNAPKAERDPRLPKPQGQTVGSFR
KGLRMLPEAISARLGSKVKLSWKLSGITKLESGGYNLTYETPDGLVSVQSKSVVMTVPSHV
ASGLLRPLSESAANALSKLYYPPVAAVSISYPKEAIRTECLIDGELKGFGQLHPRTQGVETLG
TIYSSSLFPNRAPPGRILLLNYIGGSTNTGILSKSEGELVEAVDRDLRKMLIKPNSTDPLKLGV
RVWPQAIPQFMVGHFDILDTAKSSLTSSGYEGLFLGGNYVAGVALGRCVEGAYETAIEVNN
FMSRYAYK*
Arabidopsis thaliana PPO1 L445N (SEQ ID NO: 210)
SVAGGPTVGSSKIEGGGGTTITTDCVIVGGGISGLCIAQALATKHPDAAPNLIVTEAKDRVGG
NIITREENGFLWEEGPNSFQPSDPMLTMVVDSGLKDDLVLGDPTAPRFVLWNGKLRPVPSK
LTDLPFFDLMSIGGKIRAGFGALGIRPSPPGREESVEEFVRRNLGDEVFERLIEPFCSGVYA
GDPSKLSMKAAFGKVWKLEQNGGSIIGGTFKAIQERKNAPKAERDPRLPKPQGQTVGSFR
KGLRMLPEAISARLGSKVKLSWKLSGITKLESGGYNLTYETPDGLVSVQSKSVVMTVPSHV
ASGLLRPLSESAANALSKLYYPPVAAVSISYPKEAIRTECLIDGELKGFGQLHPRTQGVETLG
TIYSSSLFPNRAPPGRILLLNYIGGSTNTGILSKSEGELVEAVDRDLRKMLIKPNSTDPLKLGV
RVWPQAIPQFNVGHFDILDTAKSSLTSSGYEGLFLGGNYVAGVALGRCVEGAYETAIEVNN
FMSRYAYK*
Arabidopsis thaliana PPO1 G476N (SEQ ID NO: 211)
SVAGGPTVGSSKIEGGGGTTITTDCVIVGGGISGLCIAQALATKHPDAAPNLIVTEAKDRVGG
NIITREENGFLWEEGPNSFQPSDPMLTMVVDSGLKDDLVLGDPTAPRFVLWNGKLRPVPSK
LTDLPFFDLMSIGGKIRAGFGALGIRPSPPGREESVEEFVRRNLGDEVFERLIEPFCSGVYA
GDPSKLSMKAAFGKVWKLEQNGGSIIGGTFKAIQERKNAPKAERDPRLPKPQGQTVGSFR
KGLRMLPEAISARLGSKVKLSWKLSGITKLESGGYNLTYETPDGLVSVQSKSVVMTVPSHV
ASGLLRPLSESAANALSKLYYPPVAAVSISYPKEAIRTECLIDGELKGFGQLHPRTQGVETLG
TIYSSSLFPNRAPPGRILLLNYIGGSTNTGILSKSEGELVEAVDRDLRKMLIKPNSTDPLKLGV
RVWPQAIPQFLVGHFDILDTAKSSLTSSGYEGLFLGGNYVANVALGRCVEGAYETAIEVNNF
MSRYAYK*
Arabidopsis thaliana PPO1 V477L (SEQ ID NO: 212)
SVAGGPTVGSSKIEGGGGTTITTDCVIVGGGISGLCIAQALATKHPDAAPNLIVTEAKDRVGG
NIITREENGFLWEEGPNSFQPSDPMLTMVVDSGLKDDLVLGDPTAPRFVLWNGKLRPVPSK
LTDLPFFDLMSIGGKIRAGFGALGIRPSPPGREESVEEFVRRNLGDEVFERLIEPFCSGVYA
GDPSKLSMKAAFGKVWKLEQNGGSIIGGTFKAIQERKNAPKAERDPRLPKPQGQTVGSFR
KGLRMLPEAISARLGSKVKLSWKLSGITKLESGGYNLTYETPDGLVSVQSKSVVMTVPSHV
ASGLLRPLSESAANALSKLYYPPVAAVSISYPKEAIRTECLIDGELKGFGQLHPRTQGVETLG
TIYSSSLFPNRAPPGRILLLNYIGGSTNTGILSKSEGELVEAVDRDLRKMLIKPNSTDPLKLGV
RVWPQAIPQFLVGHFDILDTAKSSLTSSGYEGLFLGGNYVAGLALGRCVEGAYETAIEVNNF
MSRYAYK*
Arabidopsis thaliana PPO1 V477N (SEQ ID NO: 213)
SVAGGPTVGSSKIEGGGGTTITTDCVIVGGGISGLCIAQALATKHPDAAPNLIVTEAKDRVGG
NIITREENGFLWEEGPNSFQPSDPMLTMVVDSGLKDDLVLGDPTAPRFVLWNGKLRPVPSK
LTDLPFFDLMSIGGKIRAGFGALGIRPSPPGREESVEEFVRRNLGDEVFERLIEPFCSGVYA
GDPSKLSMKAAFGKVWKLEQNGGSIIGGTFKAIQERKNAPKAERDPRLPKPQGQTVGSFR
KGLRMLPEAISARLGSKVKLSWKLSGITKLESGGYNLTYETPDGLVSVQSKSVVMTVPSHV
ASGLLRPLSESAANALSKLYYPPVAAVSISYPKEAIRTECLIDGELKGFGQLHPRTQGVETLG
TIYSSSLFPNRAPPGRILLLNYIGGSTNTGILSKSEGELVEAVDRDLRKMLIKPNSTDPLKLGV
RVWPQAIPQFLVGHFDILDTAKSSLTSSGYEGLFLGGNYVAGNALGRCVEGAYETAIEVNN
FMSRYAYK*
Arabidopsis thaliana PPO1 C482Q (SEQ ID NO: 214)
SVAGGPTVGSSKIEGGGGTTITTDCVIVGGGISGLCIAQALATKHPDAAPNLIVTEAKDRVGG
NIITREENGFLWEEGPNSFQPSDPMLTMVVDSGLKDDLVLGDPTAPRFVLWNGKLRPVPSK
LTDLPFFDLMSIGGKIRAGFGALGIRPSPPGREESVEEFVRRNLGDEVFERLIEPFCSGVYA
GDPSKLSMKAAFGKVWKLEQNGGSIIGGTFKAIQERKNAPKAERDPRLPKPQGQTVGSFR
KGLRMLPEAISARLGSKVKLSWKLSGITKLESGGYNLTYETPDGLVSVQSKSVVMTVPSHV
ASGLLRPLSESAANALSKLYYPPVAAVSISYPKEAIRTECLIDGELKGFGQLHPRTQGVETLG
TIYSSSLFPNRAPPGRILLLNYIGGSTNTGILSKSEGELVEAVDRDLRKMLIKPNSTDPLKLGV
RVWPQAIPQFLVGHFDILDTAKSSLTSSGYEGLFLGGNYVAGVALGRQVEGAYETAIEVNN
FMSRYAYK*
Setaria italica PPO1 G27A (SEQ ID NO: 215)
AAVAGGAAEAPASAGARVSADCVVVGAGISGLCTAQALATKHGVGDVLVTEARARPGGNI
TTVERPDEGYLWEEGPNSFQPSDPVLTMAVDSGLKDDLVFGDPNAPRFVLWEGKLRPVPS
KPADLPFFDLMSIPGKLRAGFGALGIRPPPPGREESVEEFVRRNLGAEVFERLIEPFCSGVY
AGDPSKLSMKAAFGKVWRLEEAGGSIIGGTIKTIQERGKNPKPPRDPRLPTPKGQTVASFR
KGLAMLPNAITSSLGSKVKLSWKLTSITKSDGMGYVLVYETPEGVVSVQAKSVIMTIPSYVA
SDILRPLSSDAADALSRFYYPPVAAVTISYPKEAIRKECLIDGELQGFGQLHPRSQGVETLGT
IYSSSLFPNRAPAGRVLLLNYIGGATNTGIVSKSASELVEAVDRDLRKMLINPSAVDPLVLGV
RVWPQAIPQFLVGHLDLLEAAKSSLDRGGYDGLFLGGNYVAGVALGRCVEGAYESASQIS
DFLTKYAYK*
Setaria italica PPO1 V49I (SEQ ID NO: 216)
AAVAGGAAEAPASAGARVSADCVVVGGGISGLCTAQALATKHGVGDVLITEARARPGGNIT
TVERPDEGYLWEEGPNSFQPSDPVLTMAVDSGLKDDLVFGDPNAPRFVLWEGKLRPVPS
KPADLPFFDLMSIPGKLRAGFGALGIRPPPPGREESVEEFVRRNLGAEVFERLIEPFCSGVY
AGDPSKLSMKAAFGKVWRLEEAGGSIIGGTIKTIQERGKNPKPPRDPRLPTPKGQTVASFR
KGLAMLPNAITSSLGSKVKLSWKLTSITKSDGMGYVLVYETPEGVVSVQAKSVIMTIPSYVA
SDILRPLSSDAADALSRFYYPPVAAVTISYPKEAIRKECLIDGELQGFGQLHPRSQGVETLGT
IYSSSLFPNRAPAGRVLLLNYIGGATNTGIVSKSASELVEAVDRDLRKMLINPSAVDPLVLGV
RVWPQAIPQFLVGHLDLLEAAKSSLDRGGYDGLFLGGNYVAGVALGRCVEGAYESASQIS
DFLTKYAYK*
Setaria italica PPO1 E74D (SEQ ID NO: 217)
AAVAGGAAEAPASAGARVSADCVVVGGGISGLCTAQALATKHGVGDVLVTEARARPGGNI
TTVERPDEGYLWEDGPNSFQPSDPVLTMAVDSGLKDDLVFGDPNAPRFVLWEGKLRPVP
SKPADLPFFDLMSIPGKLRAGFGALGIRPPPPGREESVEEFVRRNLGAEVFERLIEPFCSGV
YAGDPSKLSMKAAFGKVWRLEEAGGSIIGGTIKTIQERGKNPKPPRDPRLPTPKGQTVASF
RKGLAMLPNAITSSLGSKVKLSWKLTSITKSDGMGYVLVYETPEGVVSVQAKSVIMTIPSYV
ASDILRPLSSDAADALSRFYYPPVAAVTISYPKEAIRKECLIDGELQGFGQLHPRSQGVETLG
TIYSSSLFPNRAPAGRVLLLNYIGGATNTGIVSKSASELVEAVDRDLRKMLINPSAVDPLVLG
VRVWPQAIPQFLVGHLDLLEAAKSSLDRGGYDGLFLGGNYVAGVALGRCVEGAYESASQIS
DFLTKYAYK*
Setaria italica PPO1 E74F (SEQ ID NO: 218)
AAVAGGAAEAPASAGARVSADCVVVGGGISGLCTAQALATKHGVGDVLVTEARARPGGNI
TTVERPDEGYLWEFGPNSFQPSDPVLTMAVDSGLKDDLVFGDPNAPRFVLWEGKLRPVPS
KPADLPFFDLMSIPGKLRAGFGALGIRPPPPGREESVEEFVRRNLGAEVFERLIEPFCSGVY
AGDPSKLSMKAAFGKVWRLEEAGGSIIGGTIKTIQERGKNPKPPRDPRLPTPKGQTVASFR
KGLAMLPNAITSSLGSKVKLSWKLTSITKSDGMGYVLVYETPEGVVSVQAKSVIMTIPSYVA
SDILRPLSSDAADALSRFYYPPVAAVTISYPKEAIRKECLIDGELQGFGQLHPRSQGVETLGT
IYSSSLFPNRAPAGRVLLLNYIGGATNTGIVSKSASELVEAVDRDLRKMLINPSAVDPLVLGV
RVWPQAIPQFLVGHLDLLEAAKSSLDRGGYDGLFLGGNYVAGVALGRCVEGAYESASQIS
DFLTKYAYK*
Setaria italica PPO1 V90I (SEQ ID NO: 219)
AAVAGGAAEAPASAGARVSADCVVVGGGISGLCTAQALATKHGVGDVLVTEARARPGGNI
TTVERPDEGYLWEEGPNSFQPSDPVLTMAIDSGLKDDLVFGDPNAPRFVLWEGKLRPVPS
KPADLPFFDLMSIPGKLRAGFGALGIRPPPPGREESVEEFVRRNLGAEVFERLIEPFCSGVY
AGDPSKLSMKAAFGKVWRLEEAGGSIIGGTIKTIQERGKNPKPPRDPRLPTPKGQTVASFR
KGLAMLPNAITSSLGSKVKLSWKLTSITKSDGMGYVLVYETPEGVVSVQAKSVIMTIPSYVA
SDILRPLSSDAADALSRFYYPPVAAVTISYPKEAIRKECLIDGELQGFGQLHPRSQGVETLGT
IYSSSLFPNRAPAGRVLLLNYIGGATNTGIVSKSASELVEAVDRDLRKMLINPSAVDPLVLGV
RVWPQAIPQFLVGHLDLLEAAKSSLDRGGYDGLFLGGNYVAGVALGRCVEGAYESASQIS
DFLTKYAYK*
Setaria italica PPO1 V90L (SEQ ID NO: 220)
AAVAGGAAEAPASAGARVSADCVVVGGGISGLCTAQALATKHGVGDVLVTEARARPGGNI
TTVERPDEGYLWEEGPNSFQPSDPVLTMALDSGLKDDLVFGDPNAPRFVLWEGKLRPVPS
KPADLPFFDLMSIPGKLRAGFGALGIRPPPPGREESVEEFVRRNLGAEVFERLIEPFCSGVY
AGDPSKLSMKAAFGKVWRLEEAGGSIIGGTIKTIQERGKNPKPPRDPRLPTPKGQTVASFR
KGLAMLPNAITSSLGSKVKLSWKLTSITKSDGMGYVLVYETPEGVVSVQAKSVIMTIPSYVA
SDILRPLSSDAADALSRFYYPPVAAVTISYPKEAIRKECLIDGELQGFGQLHPRSQGVETLGT
IYSSSLFPNRAPAGRVLLLNYIGGATNTGIVSKSASELVEAVDRDLRKMLINPSAVDPLVLGV
RVWPQAIPQFLVGHLDLLEAAKSSLDRGGYDGLFLGGNYVAGVALGRCVEGAYESASQIS
DFLTKYAYK*
Setaria italica PPO1 P117Y (SEQ ID NO: 221)
AAVAGGAAEAPASAGARVSADCVVVGGGISGLCTAQALATKHGVGDVLVTEARARPGGNI
TTVERPDEGYLWEEGPNSFQPSDPVLTMAVDSGLKDDLVFGDPNAPRFVLWEGKLRYVPS
KPADLPFFDLMSIPGKLRAGFGALGIRPPPPGREESVEEFVRRNLGAEVFERLIEPFCSGVY
AGDPSKLSMKAAFGKVWRLEEAGGSIIGGTIKTIQERGKNPKPPRDPRLPTPKGQTVASFR
KGLAMLPNAITSSLGSKVKLSWKLTSITKSDGMGYVLVYETPEGVVSVQAKSVIMTIPSYVA
SDILRPLSSDAADALSRFYYPPVAAVTISYPKEAIRKECLIDGELQGFGQLHPRSQGVETLGT
IYSSSLFPNRAPAGRVLLLNYIGGATNTGIVSKSASELVEAVDRDLRKMLINPSAVDPLVLGV
RVWPQAIPQFLVGHLDLLEAAKSSLDRGGYDGLFLGGNYVAGVALGRCVEGAYESASQIS
DFLTKYAYK*
Setaria italica PPO1 K136H (SEQ ID NO: 222)
AAVAGGAAEAPASAGARVSADCVVVGGGISGLCTAQALATKHGVGDVLVTEARARPGGNI
TTVERPDEGYLWEEGPNSFQPSDPVLTMAVDSGLKDDLVFGDPNAPRFVLWEGKLRPVPS
KPADLPFFDLMSIPGHLRAGFGALGIRPPPPGREESVEEFVRRNLGAEVFERLIEPFCSGVY
AGDPSKLSMKAAFGKVWRLEEAGGSIIGGTIKTIQERGKNPKPPRDPRLPTPKGQTVASFR
KGLAMLPNAITSSLGSKVKLSWKLTSITKSDGMGYVLVYETPEGVVSVQAKSVIMTIPSYVA
SDILRPLSSDAADALSRFYYPPVAAVTISYPKEAIRKECLIDGELQGFGQLHPRSQGVETLGT
IYSSSLFPNRAPAGRVLLLNYIGGATNTGIVSKSASELVEAVDRDLRKMLINPSAVDPLVLGV
RVWPQAIPQFLVGHLDLLEAAKSSLDRGGYDGLFLGGNYVAGVALGRCVEGAYESASQIS
DFLTKYAYK*
Setaria italica PPO1 A139L (SEQ ID NO: 223)
AAVAGGAAEAPASAGARVSADCVVVGGGISGLCTAQALATKHGVGDVLVTEARARPGGNI
TTVERPDEGYLWEEGPNSFQPSDPVLTMAVDSGLKDDLVFGDPNAPRFVLWEGKLRPVPS
KPADLPFFDLMSIPGKLRLGFGALGIRPPPPGREESVEEFVRRNLGAEVFERLIEPFCSGVY
AGDPSKLSMKAAFGKVWRLEEAGGSIIGGTIKTIQERGKNPKPPRDPRLPTPKGQTVASFR
KGLAMLPNAITSSLGSKVKLSWKLTSITKSDGMGYVLVYETPEGVVSVQAKSVIMTIPSYVA
SDILRPLSSDAADALSRFYYPPVAAVTISYPKEAIRKECLIDGELQGFGQLHPRSQGVETLGT
IYSSSLFPNRAPAGRVLLLNYIGGATNTGIVSKSASELVEAVDRDLRKMLINPSAVDPLVLGV
RVWPQAIPQFLVGHLDLLEAAKSSLDRGGYDGLFLGGNYVAGVALGRCVEGAYESASQIS
DFLTKYAYK*
Setaria italica PPO1 V161W (SEQ ID NO: 224)
AAVAGGAAEAPASAGARVSADCVVVGGGISGLCTAQALATKHGVGDVLVTEARARPGGNI
TTVERPDEGYLWEEGPNSFQPSDPVLTMAVDSGLKDDLVFGDPNAPRFVLWEGKLRPVPS
KPADLPFFDLMSIPGKLRAGFGALGIRPPPPGREESVEEFWRRNLGAEVFERLIEPFCSGVY
AGDPSKLSMKAAFGKVWRLEEAGGSIIGGTIKTIQERGKNPKPPRDPRLPTPKGQTVASFR
KGLAMLPNAITSSLGSKVKLSWKLTSITKSDGMGYVLVYETPEGVVSVQAKSVIMTIPSYVA
SDILRPLSSDAADALSRFYYPPVAAVTISYPKEAIRKECLIDGELQGFGQLHPRSQGVETLGT
IYSSSLFPNRAPAGRVLLLNYIGGATNTGIVSKSASELVEAVDRDLRKMLINPSAVDPLVLGV
RVWPQAIPQFLVGHLDLLEAAKSSLDRGGYDGLFLGGNYVAGVALGRCVEGAYESASQIS
DFLTKYAYK*
Setaria italica PPO1 V169G (SEQ ID NO: 225)
AAVAGGAAEAPASAGARVSADCVVVGGGISGLCTAQALATKHGVGDVLVTEARARPGGNI
TTVERPDEGYLWEEGPNSFQPSDPVLTMAVDSGLKDDLVFGDPNAPRFVLWEGKLRPVPS
KPADLPFFDLMSIPGKLRAGFGALGIRPPPPGREESVEEFVRRNLGAEGFERLIEPFCSGVY
AGDPSKLSMKAAFGKVWRLEEAGGSIIGGTIKTIQERGKNPKPPRDPRLPTPKGQTVASFR
KGLAMLPNAITSSLGSKVKLSWKLTSITKSDGMGYVLVYETPEGVVSVQAKSVIMTIPSYVA
SDILRPLSSDAADALSRFYYPPVAAVTISYPKEAIRKECLIDGELQGFGQLHPRSQGVETLGT
IYSSSLFPNRAPAGRVLLLNYIGGATNTGIVSKSASELVEAVDRDLRKMLINPSAVDPLVLGV
RVWPQAIPQFLVGHLDLLEAAKSSLDRGGYDGLFLGGNYVAGVALGRCVEGAYESASQIS
DFLTKYAYK*
Setaria italica PPO1 F170Y (SEQ ID NO: 226)
AAVAGGAAEAPASAGARVSADCVVVGGGISGLCTAQALATKHGVGDVLVTEARARPGGNI
TTVERPDEGYLWEEGPNSFQPSDPVLTMAVDSGLKDDLVFGDPNAPRFVLWEGKLRPVPS
KPADLPFFDLMSIPGKLRAGFGALGIRPPPPGREESVEEFVRRNLGAEVYERLIEPFCSGVY
AGDPSKLSMKAAFGKVWRLEEAGGSIIGGTIKTIQERGKNPKPPRDPRLPTPKGQTVASFR
KGLAMLPNAITSSLGSKVKLSWKLTSITKSDGMGYVLVYETPEGVVSVQAKSVIMTIPSYVA
SDILRPLSSDAADALSRFYYPPVAAVTISYPKEAIRKECLIDGELQGFGQLHPRSQGVETLGT
IYSSSLFPNRAPAGRVLLLNYIGGATNTGIVSKSASELVEAVDRDLRKMLINPSAVDPLVLGV
RVWPQAIPQFLVGHLDLLEAAKSSLDRGGYDGLFLGGNYVAGVALGRCVEGAYESASQIS
DFLTKYAYK*
Setaria italica PPO1 E175Y (SEQ ID NO: 227)
AAVAGGAAEAPASAGARVSADCVVVGGGISGLCTAQALATKHGVGDVLVTEARARPGGNI
TTVERPDEGYLWEEGPNSFQPSDPVLTMAVDSGLKDDLVFGDPNAPRFVLWEGKLRPVPS
KPADLPFFDLMSIPGKLRAGFGALGIRPPPPGREESVEEFVRRNLGAEVFERLIYPFCSGVY
AGDPSKLSMKAAFGKVWRLEEAGGSIIGGTIKTIQERGKNPKPPRDPRLPTPKGQTVASFR
KGLAMLPNAITSSLGSKVKLSWKLTSITKSDGMGYVLVYETPEGVVSVQAKSVIMTIPSYVA
SDILRPLSSDAADALSRFYYPPVAAVTISYPKEAIRKECLIDGELQGFGQLHPRSQGVETLGT
IYSSSLFPNRAPAGRVLLLNYIGGATNTGIVSKSASELVEAVDRDLRKMLINPSAVDPLVLGV
RVWPQAIPQFLVGHLDLLEAAKSSLDRGGYDGLFLGGNYVAGVALGRCVEGAYESASQIS
DFLTKYAYK*
Setaria italica PPO1 F177Y (SEQ ID NO: 228)
AAVAGGAAEAPASAGARVSADCVVVGGGISGLCTAQALATKHGVGDVLVTEARARPGGNI
TTVERPDEGYLWEEGPNSFQPSDPVLTMAVDSGLKDDLVFGDPNAPRFVLWEGKLRPVPS
KPADLPFFDLMSIPGKLRAGFGALGIRPPPPGREESVEEFVRRNLGAEVFERLIEPYCSGVY
AGDPSKLSMKAAFGKVWRLEEAGGSIIGGTIKTIQERGKNPKPPRDPRLPTPKGQTVASFR
KGLAMLPNAITSSLGSKVKLSWKLTSITKSDGMGYVLVYETPEGVVSVQAKSVIMTIPSYVA
SDILRPLSSDAADALSRFYYPPVAAVTISYPKEAIRKECLIDGELQGFGQLHPRSQGVETLGT
IYSSSLFPNRAPAGRVLLLNYIGGATNTGIVSKSASELVEAVDRDLRKMLINPSAVDPLVLGV
RVWPQAIPQFLVGHLDLLEAAKSSLDRGGYDGLFLGGNYVAGVALGRCVEGAYESASQIS
DFLTKYAYK*
Setaria italica PPO1 V181K (SEQ ID NO: 229)
AAVAGGAAEAPASAGARVSADCVVVGGGISGLCTAQALATKHGVGDVLVTEARARPGGNI
TTVERPDEGYLWEEGPNSFQPSDPVLTMAVDSGLKDDLVFGDPNAPRFVLWEGKLRPVPS
KPADLPFFDLMSIPGKLRAGFGALGIRPPPPGREESVEEFVRRNLGAEVFERLIEPFCSGKY
AGDPSKLSMKAAFGKVWRLEEAGGSIIGGTIKTIQERGKNPKPPRDPRLPTPKGQTVASFR
KGLAMLPNAITSSLGSKVKLSWKLTSITKSDGMGYVLVYETPEGVVSVQAKSVIMTIPSYVA
SDILRPLSSDAADALSRFYYPPVAAVTISYPKEAIRKECLIDGELQGFGQLHPRSQGVETLGT
IYSSSLFPNRAPAGRVLLLNYIGGATNTGIVSKSASELVEAVDRDLRKMLINPSAVDPLVLGV
RVWPQAIPQFLVGHLDLLEAAKSSLDRGGYDGLFLGGNYVAGVALGRCVEGAYESASQIS
DFLTKYAYK*
Setaria italica PPO1 Y182S (SEQ ID NO: 230)
AAVAGGAAEAPASAGARVSADCVVVGGGISGLCTAQALATKHGVGDVLVTEARARPGGNI
TTVERPDEGYLWEEGPNSFQPSDPVLTMAVDSGLKDDLVFGDPNAPRFVLWEGKLRPVPS
KPADLPFFDLMSIPGKLRAGFGALGIRPPPPGREESVEEFVRRNLGAEVFERLIEPFCSGVS
AGDPSKLSMKAAFGKVWRLEEAGGSIIGGTIKTIQERGKNPKPPRDPRLPTPKGQTVASFR
KGLAMLPNAITSSLGSKVKLSWKLTSITKSDGMGYVLVYETPEGVVSVQAKSVIMTIPSYVA
SDILRPLSSDAADALSRFYYPPVAAVTISYPKEAIRKECLIDGELQGFGQLHPRSQGVETLGT
IYSSSLFPNRAPAGRVLLLNYIGGATNTGIVSKSASELVEAVDRDLRKMLINPSAVDPLVLGV
RVWPQAIPQFLVGHLDLLEAAKSSLDRGGYDGLFLGGNYVAGVALGRCVEGAYESASQIS
DFLTKYAYK*
Setaria italica PPO1 A183C (SEQ ID NO: 231)
AAVAGGAAEAPASAGARVSADCVVVGGGISGLCTAQALATKHGVGDVLVTEARARPGGNI
TTVERPDEGYLWEEGPNSFQPSDPVLTMAVDSGLKDDLVFGDPNAPRFVLWEGKLRPVPS
KPADLPFFDLMSIPGKLRAGFGALGIRPPPPGREESVEEFVRRNLGAEVFERLIEPFCSGVY
CAGDPSKLSMKAAFGKVWRLEEAGGSIIGGTIKTIQERGKNPKPPRDPRLPTPKGQTVASF
RKGLAMLPNAITSSLGSKVKLSWKLTSITKSDGMGYVLVYETPEGVVSVQAKSVIMTIPSYV
ASDILRPLSSDAADALSRFYYPPVAAVTISYPKEAIRKECLIDGELQGFGQLHPRSQGVETLG
TIYSSSLFPNRAPAGRVLLLNYIGGATNTGIVSKSASELVEAVDRDLRKMLINPSAVDPLVLG
VRVWPQAIPQFLVGHLDLLEAAKSSLDRGGYDGLFLGGNYVAGVALGRCVEGAYESASQIS
DFLTKYAYK*
Setaria italica PPO1 P234C (SEQ ID NO: 232)
AAVAGGAAEAPASAGARVSADCVVVGGGISGLCTAQALATKHGVGDVLVTEARARPGGNI
TTVERPDEGYLWEEGPNSFQPSDPVLTMAVDSGLKDDLVFGDPNAPRFVLWEGKLRPVPS
KPADLPFFDLMSIPGKLRAGFGALGIRPPPPGREESVEEFVRRNLGAEVFERLIEPFCSGVY
AGDPSKLSMKAAFGKVWRLEEAGGSIIGGTIKTIQERGKNPKPPRDPRLPTCKGQTVASFR
KGLAMLPNAITSSLGSKVKLSWKLTSITKSDGMGYVLVYETPEGVVSVQAKSVIMTIPSYVA
SDILRPLSSDAADALSRFYYPPVAAVTISYPKEAIRKECLIDGELQGFGQLHPRSQGVETLGT
IYSSSLFPNRAPAGRVLLLNYIGGATNTGIVSKSASELVEAVDRDLRKMLINPSAVDPLVLGV
RVWPQAIPQFLVGHLDLLEAAKSSLDRGGYDGLFLGGNYVAGVALGRCVEGAYESASQIS
DFLTKYAYK*
Setaria italica PPO1 K235H (SEQ ID NO: 233)
AAVAGGAAEAPASAGARVSADCVVVGGGISGLCTAQALATKHGVGDVLVTEARARPGGNI
TTVERPDEGYLWEEGPNSFQPSDPVLTMAVDSGLKDDLVFGDPNAPRFVLWEGKLRPVPS
KPADLPFFDLMSIPGKLRAGFGALGIRPPPPGREESVEEFVRRNLGAEVFERLIEPFCSGVY
AGDPSKLSMKAAFGKVWRLEEAGGSIIGGTIKTIQERGKNPKPPRDPRLPTPHGQTVASFR
KGLAMLPNAITSSLGSKVKLSWKLTSITKSDGMGYVLVYETPEGVVSVQAKSVIMTIPSYVA
SDILRPLSSDAADALSRFYYPPVAAVTISYPKEAIRKECLIDGELQGFGQLHPRSQGVETLGT
IYSSSLFPNRAPAGRVLLLNYIGGATNTGIVSKSASELVEAVDRDLRKMLINPSAVDPLVLGV
RVWPQAIPQFLVGHLDLLEAAKSSLDRGGYDGLFLGGNYVAGVALGRCVEGAYESASQIS
DFLTKYAYK*
Setaria italica PPO1 K235I (SEQ ID NO: 234)
AAVAGGAAEAPASAGARVSADCVVVGGGISGLCTAQALATKHGVGDVLVTEARARPGGNI
TTVERPDEGYLWEEGPNSFQPSDPVLTMAVDSGLKDDLVFGDPNAPRFVLWEGKLRPVPS
KPADLPFFDLMSIPGKLRAGFGALGIRPPPPGREESVEEFVRRNLGAEVFERLIEPFCSGVY
AGDPSKLSMKAAFGKVWRLEEAGGSIIGGTIKTIQERGKNPKPPRDPRLPTPIGQTVASFRK
GLAMLPNAITSSLGSKVKLSWKLTSITKSDGMGYVLVYETPEGVVSVQAKSVIMTIPSYVAS
DILRPLSSDAADALSRFYYPPVAAVTISYPKEAIRKECLIDGELQGFGQLHPRSQGVETLGTI
YSSSLFPNRAPAGRVLLLNYIGGATNTGIVSKSASELVEAVDRDLRKMLINPSAVDPLVLGV
RVWPQAIPQFLVGHLDLLEAAKSSLDRGGYDGLFLGGNYVAGVALGRCVEGAYESASQIS
DFLTKYAYK*
Setaria italica PPO1 K235T (SEQ ID NO: 235)
AAVAGGAAEAPASAGARVSADCVVVGGGISGLCTAQALATKHGVGDVLVTEARARPGGNI
TTVERPDEGYLWEEGPNSFQPSDPVLTMAVDSGLKDDLVFGDPNAPRFVLWEGKLRPVPS
KPADLPFFDLMSIPGKLRAGFGALGIRPPPPGREESVEEFVRRNLGAEVFERLIEPFCSGVY
AGDPSKLSMKAAFGKVWRLEEAGGSIIGGTIKTIQERGKNPKPPRDPRLPTPTGQTVASFR
KGLAMLPNAITSSLGSKVKLSWKLTSITKSDGMGYVLVYETPEGVVSVQAKSVIMTIPSYVA
SDILRPLSSDAADALSRFYYPPVAAVTISYPKEAIRKECLIDGELQGFGQLHPRSQGVETLGT
IYSSSLFPNRAPAGRVLLLNYIGGATNTGIVSKSASELVEAVDRDLRKMLINPSAVDPLVLGV
RVWPQAIPQFLVGHLDLLEAAKSSLDRGGYDGLFLGGNYVAGVALGRCVEGAYESASQIS
DFLTKYAYK*
Setaria italica PPO1 K244C (SEQ ID NO: 236)
AAVAGGAAEAPASAGARVSADCVVVGGGISGLCTAQALATKHGVGDVLVTEARARPGGNI
TTVERPDEGYLWEEGPNSFQPSDPVLTMAVDSGLKDDLVFGDPNAPRFVLWEGKLRPVPS
KPADLPFFDLMSIPGKLRAGFGALGIRPPPPGREESVEEFVRRNLGAEVFERLIEPFCSGVY
AGDPSKLSMKAAFGKVWRLEEAGGSIIGGTIKTIQERGKNPKPPRDPRLPTPKGQTVASFR
CGLAMLPNAITSSLGSKVKLSWKLTSITKSDGMGYVLVYETPEGVVSVQAKSVIMTIPSYVA
SDILRPLSSDAADALSRFYYPPVAAVTISYPKEAIRKECLIDGELQGFGQLHPRSQGVETLGT
IYSSSLFPNRAPAGRVLLLNYIGGATNTGIVSKSASELVEAVDRDLRKMLINPSAVDPLVLGV
RVWPQAIPQFLVGHLDLLEAAKSSLDRGGYDGLFLGGNYVAGVALGRCVEGAYESASQIS
DFLTKYAYK*
Setaria italica PPO1 K244E (SEQ ID NO: 237)
AAVAGGAAEAPASAGARVSADCVVVGGGISGLCTAQALATKHGVGDVLVTEARARPGGNI
TTVERPDEGYLWEEGPNSFQPSDPVLTMAVDSGLKDDLVFGDPNAPRFVLWEGKLRPVPS
KPADLPFFDLMSIPGKLRAGFGALGIRPPPPGREESVEEFVRRNLGAEVFERLIEPFCSGVY
AGDPSKLSMKAAFGKVWRLEEAGGSIIGGTIKTIQERGKNPKPPRDPRLPTPKGQTVASFR
EGLAMLPNAITSSLGSKVKLSWKLTSITKSDGMGYVLVYETPEGVVSVQAKSVIMTIPSYVA
SDILRPLSSDAADALSRFYYPPVAAVTISYPKEAIRKECLIDGELQGFGQLHPRSQGVETLGT
IYSSSLFPNRAPAGRVLLLNYIGGATNTGIVSKSASELVEAVDRDLRKMLINPSAVDPLVLGV
RVWPQAIPQFLVGHLDLLEAAKSSLDRGGYDGLFLGGNYVAGVALGRCVEGAYESASQIS
DFLTKYAYK*
Setaria italica PPO1 K244N (SEQ ID NO: 238)
AAVAGGAAEAPASAGARVSADCVVVGGGISGLCTAQALATKHGVGDVLVTEARARPGGNI
TTVERPDEGYLWEEGPNSFQPSDPVLTMAVDSGLKDDLVFGDPNAPRFVLWEGKLRPVPS
KPADLPFFDLMSIPGKLRAGFGALGIRPPPPGREESVEEFVRRNLGAEVFERLIEPFCSGVY
AGDPSKLSMKAAFGKVWRLEEAGGSIIGGTIKTIQERGKNPKPPRDPRLPTPKGQTVASFR
NGLAMLPNAITSSLGSKVKLSWKLTSITKSDGMGYVLVYETPEGVVSVQAKSVIMTIPSYVA
SDILRPLSSDAADALSRFYYPPVAAVTISYPKEAIRKECLIDGELQGFGQLHPRSQGVETLGT
IYSSSLFPNRAPAGRVLLLNYIGGATNTGIVSKSASELVEAVDRDLRKMLINPSAVDPLVLGV
RVWPQAIPQFLVGHLDLLEAAKSSLDRGGYDGLFLGGNYVAGVALGRCVEGAYESASQIS
DFLTKYAYK*
Setaria italica PPO1 A247G (SEQ ID NO: 239)
AAVAGGAAEAPASAGARVSADCVVVGGGISGLCTAQALATKHGVGDVLVTEARARPGGNI
TTVERPDEGYLWEEGPNSFQPSDPVLTMAVDSGLKDDLVFGDPNAPRFVLWEGKLRPVPS
KPADLPFFDLMSIPGKLRAGFGALGIRPPPPGREESVEEFVRRNLGAEVFERLIEPFCSGVY
AGDPSKLSMKAAFGKVWRLEEAGGSIIGGTIKTIQERGKNPKPPRDPRLPTPKGQTVASFR
KGLGMLPNAITSSLGSKVKLSWKLTSITKSDGMGYVLVYETPEGVVSVQAKSVIMTIPSYVA
SDILRPLSSDAADALSRFYYPPVAAVTISYPKEAIRKECLIDGELQGFGQLHPRSQGVETLGT
IYSSSLFPNRAPAGRVLLLNYIGGATNTGIVSKSASELVEAVDRDLRKMLINPSAVDPLVLGV
RVWPQAIPQFLVGHLDLLEAAKSSLDRGGYDGLFLGGNYVAGVALGRCVEGAYESASQIS
DFLTKYAYK*
Setaria italica PPO1 T268L (SEQ ID NO: 240)
AAVAGGAAEAPASAGARVSADCVVVGGGISGLCTAQALATKHGVGDVLVTEARARPGGNI
TTVERPDEGYLWEEGPNSFQPSDPVLTMAVDSGLKDDLVFGDPNAPRFVLWEGKLRPVPS
KPADLPFFDLMSIPGKLRAGFGALGIRPPPPGREESVEEFVRRNLGAEVFERLIEPFCSGVY
AGDPSKLSMKAAFGKVWRLEEAGGSIIGGTIKTIQERGKNPKPPRDPRLPTPKGQTVASFR
KGLAMLPNAITSSLGSKVKLSWKLLSITKSDGMGYVLVYETPEGVVSVQAKSVIMTIPSYVAS
DILRPLSSDAADALSRFYYPPVAAVTISYPKEAIRKECLIDGELQGFGQLHPRSQGVETLGTI
YSSSLFPNRAPAGRVLLLNYIGGATNTGIVSKSASELVEAVDRDLRKMLINPSAVDPLVLGV
RVWPQAIPQFLVGHLDLLEAAKSSLDRGGYDGLFLGGNYVAGVALGRCVEGAYESASQIS
DFLTKYAYK*
Setaria italica PPO1 F323C (SEQ ID NO: 241)
AAVAGGAAEAPASAGARVSADCVVVGGGISGLCTAQALATKHGVGDVLVTEARARPGGNI
TTVERPDEGYLWEEGPNSFQPSDPVLTMAVDSGLKDDLVFGDPNAPRFVLWEGKLRPVPS
KPADLPFFDLMSIPGKLRAGFGALGIRPPPPGREESVEEFVRRNLGAEVFERLIEPFCSGVY
AGDPSKLSMKAAFGKVWRLEEAGGSIIGGTIKTIQERGKNPKPPRDPRLPTPKGQTVASFR
KGLAMLPNAITSSLGSKVKLSWKLTSITKSDGMGYVLVYETPEGVVSVQAKSVIMTIPSYVA
SDILRPLSSDAADALSRCYYPPVAAVTISYPKEAIRKECLIDGELQGFGQLHPRSQGVETLGT
IYSSSLFPNRAPAGRVLLLNYIGGATNTGIVSKSASELVEAVDRDLRKMLINPSAVDPLVLGV
RVWPQAIPQFLVGHLDLLEAAKSSLDRGGYDGLFLGGNYVAGVALGRCVEGAYESASQIS
DFLTKYAYK*
Setaria italica PPO1 Y324D (SEQ ID NO: 242)
AAVAGGAAEAPASAGARVSADCVVVGGGISGLCTAQALATKHGVGDVLVTEARARPGGNI
TTVERPDEGYLWEEGPNSFQPSDPVLTMAVDSGLKDDLVFGDPNAPRFVLWEGKLRPVPS
KPADLPFFDLMSIPGKLRAGFGALGIRPPPPGREESVEEFVRRNLGAEVFERLIEPFCSGVY
AGDPSKLSMKAAFGKVWRLEEAGGSIIGGTIKTIQERGKNPKPPRDPRLPTPKGQTVASFR
KGLAMLPNAITSSLGSKVKLSWKLTSITKSDGMGYVLVYETPEGVVSVQAKSVIMTIPSYVA
SDILRPLSSDAADALSRFDYPPVAAVTISYPKEAIRKECLIDGELQGFGQLHPRSQGVETLGT
IYSSSLFPNRAPAGRVLLLNYIGGATNTGIVSKSASELVEAVDRDLRKMLINPSAVDPLVLGV
RVWPQAIPQFLVGHLDLLEAAKSSLDRGGYDGLFLGGNYVAGVALGRCVEGAYESASQIS
DFLTKYAYK*
Setaria italica PPO1 Y324F (SEQ ID NO: 243)
AAVAGGAAEAPASAGARVSADCVVVGGGISGLCTAQALATKHGVGDVLVTEARARPGGNI
TTVERPDEGYLWEEGPNSFQPSDPVLTMAVDSGLKDDLVFGDPNAPRFVLWEGKLRPVPS
KPADLPFFDLMSIPGKLRAGFGALGIRPPPPGREESVEEFVRRNLGAEVFERLIEPFCSGVY
AGDPSKLSMKAAFGKVWRLEEAGGSIIGGTIKTIQERGKNPKPPRDPRLPTPKGQTVASFR
KGLAMLPNAITSSLGSKVKLSWKLTSITKSDGMGYVLVYETPEGVVSVQAKSVIMTIPSYVA
SDILRPLSSDAADALSRFFYPPVAAVTISYPKEAIRKECLIDGELQGFGQLHPRSQGVETLGT
IYSSSLFPNRAPAGRVLLLNYIGGATNTGIVSKSASELVEAVDRDLRKMLINPSAVDPLVLGV
RVWPQAIPQFLVGHLDLLEAAKSSLDRGGYDGLFLGGNYVAGVALGRCVEGAYESASQIS
DFLTKYAYK*
Setaria italica PPO1 Y324P (SEQ ID NO: 244)
AAVAGGAAEAPASAGARVSADCVVVGGGISGLCTAQALATKHGVGDVLVTEARARPGGNI
TTVERPDEGYLWEEGPNSFQPSDPVLTMAVDSGLKDDLVFGDPNAPRFVLWEGKLRPVPS
KPADLPFFDLMSIPGKLRAGFGALGIRPPPPGREESVEEFVRRNLGAEVFERLIEPFCSGVY
AGDPSKLSMKAAFGKVWRLEEAGGSIIGGTIKTIQERGKNPKPPRDPRLPTPKGQTVASFR
KGLAMLPNAITSSLGSKVKLSWKLTSITKSDGMGYVLVYETPEGVVSVQAKSVIMTIPSYVA
SDILRPLSSDAADALSRFPYPPVAAVTISYPKEAIRKECLIDGELQGFGQLHPRSQGVETLGT
IYSSSLFPNRAPAGRVLLLNYIGGATNTGIVSKSASELVEAVDRDLRKMLINPSAVDPLVLGV
RVWPQAIPQFLVGHLDLLEAAKSSLDRGGYDGLFLGGNYVAGVALGRCVEGAYESASQIS
DFLTKYAYK*
Setaria italica PPO1 Y324Q (SEQ ID NO: 245)
AAVAGGAAEAPASAGARVSADCVVVGGGISGLCTAQALATKHGVGDVLVTEARARPGGNI
TTVERPDEGYLWEEGPNSFQPSDPVLTMAVDSGLKDDLVFGDPNAPRFVLWEGKLRPVPS
KPADLPFFDLMSIPGKLRAGFGALGIRPPPPGREESVEEFVRRNLGAEVFERLIEPFCSGVY
AGDPSKLSMKAAFGKVWRLEEAGGSIIGGTIKTIQERGKNPKPPRDPRLPTPKGQTVASFR
KGLAMLPNAITSSLGSKVKLSWKLTSITKSDGMGYVLVYETPEGVVSVQAKSVIMTIPSYVA
SDILRPLSSDAADALSRFQYPPVAAVTISYPKEAIRKECLIDGELQGFGQLHPRSQGVETLGT
IYSSSLFPNRAPAGRVLLLNYIGGATNTGIVSKSASELVEAVDRDLRKMLINPSAVDPLVLGV
RVWPQAIPQFLVGHLDLLEAAKSSLDRGGYDGLFLGGNYVAGVALGRCVEGAYESASQIS
DFLTKYAYK*
Setaria italica PPO1 Y324R (SEQ ID NO: 246)
AAVAGGAAEAPASAGARVSADCVVVGGGISGLCTAQALATKHGVGDVLVTEARARPGGNI
TTVERPDEGYLWEEGPNSFQPSDPVLTMAVDSGLKDDLVFGDPNAPRFVLWEGKLRPVPS
KPADLPFFDLMSIPGKLRAGFGALGIRPPPPGREESVEEFVRRNLGAEVFERLIEPFCSGVY
AGDPSKLSMKAAFGKVWRLEEAGGSIIGGTIKTIQERGKNPKPPRDPRLPTPKGQTVASFR
KGLAMLPNAITSSLGSKVKLSWKLTSITKSDGMGYVLVYETPEGVVSVQAKSVIMTIPSYVA
SDILRPLSSDAADALSRFRYPPVAAVTISYPKEAIRKECLIDGELQGFGQLHPRSQGVETLGT
IYSSSLFPNRAPAGRVLLLNYIGGATNTGIVSKSASELVEAVDRDLRKMLINPSAVDPLVLGV
RVWPQAIPQFLVGHLDLLEAAKSSLDRGGYDGLFLGGNYVAGVALGRCVEGAYESASQIS
DFLTKYAYK*
Setaria italica PPO1 Y324T (SEQ ID NO: 247)
AAVAGGAAEAPASAGARVSADCVVVGGGISGLCTAQALATKHGVGDVLVTEARARPGGNI
TTVERPDEGYLWEEGPNSFQPSDPVLTMAVDSGLKDDLVFGDPNAPRFVLWEGKLRPVPS
KPADLPFFDLMSIPGKLRAGFGALGIRPPPPGREESVEEFVRRNLGAEVFERLIEPFCSGVY
AGDPSKLSMKAAFGKVWRLEEAGGSIIGGTIKTIQERGKNPKPPRDPRLPTPKGQTVASFR
KGLAMLPNAITSSLGSKVKLSWKLTSITKSDGMGYVLVYETPEGVVSVQAKSVIMTIPSYVA
SDILRPLSSDAADALSRFTYPPVAAVTISYPKEAIRKECLIDGELQGFGQLHPRSQGVETLGT
IYSSSLFPNRAPAGRVLLLNYIGGATNTGIVSKSASELVEAVDRDLRKMLINPSAVDPLVLGV
RVWPQAIPQFLVGHLDLLEAAKSSLDRGGYDGLFLGGNYVAGVALGRCVEGAYESASQIS
DFLTKYAYK*
Setaria italica PPO1 Y325F (SEQ ID NO: 248)
AAVAGGAAEAPASAGARVSADCVVVGGGISGLCTAQALATKHGVGDVLVTEARARPGGNI
TTVERPDEGYLWEEGPNSFQPSDPVLTMAVDSGLKDDLVFGDPNAPRFVLWEGKLRPVPS
KPADLPFFDLMSIPGKLRAGFGALGIRPPPPGREESVEEFVRRNLGAEVFERLIEPFCSGVY
AGDPSKLSMKAAFGKVWRLEEAGGSIIGGTIKTIQERGKNPKPPRDPRLPTPKGQTVASFR
KGLAMLPNAITSSLGSKVKLSWKLTSITKSDGMGYVLVYETPEGVVSVQAKSVIMTIPSYVA
SDILRPLSSDAADALSRFYFPPVAAVTISYPKEAIRKECLIDGELQGFGQLHPRSQGVETLGT
IYSSSLFPNRAPAGRVLLLNYIGGATNTGIVSKSASELVEAVDRDLRKMLINPSAVDPLVLGV
RVWPQAIPQFLVGHLDLLEAAKSSLDRGGYDGLFLGGNYVAGVALGRCVEGAYESASQIS
DFLTKYAYK*
Setaria italica PPO1 V328L (SEQ ID NO: 249)
AAVAGGAAEAPASAGARVSADCVVVGGGISGLCTAQALATKHGVGDVLVTEARARPGGNI
TTVERPDEGYLWEEGPNSFQPSDPVLTMAVDSGLKDDLVFGDPNAPRFVLWEGKLRPVPS
KPADLPFFDLMSIPGKLRAGFGALGIRPPPPGREESVEEFVRRNLGAEVFERLIEPFCSGVY
AGDPSKLSMKAAFGKVWRLEEAGGSIIGGTIKTIQERGKNPKPPRDPRLPTPKGQTVASFR
KGLAMLPNAITSSLGSKVKLSWKLTSITKSDGMGYVLVYETPEGVVSVQAKSVIMTIPSYVA
SDILRPLSSDAADALSRFYYPPLAAVTISYPKEAIRKECLIDGELQGFGQLHPRSQGVETLGTI
YSSSLFPNRAPAGRVLLLNYIGGATNTGIVSKSASELVEAVDRDLRKMLINPSAVDPLVLGV
RVWPQAIPQFLVGHLDLLEAAKSSLDRGGYDGLFLGGNYVAGVALGRCVEGAYESASQIS
DFLTKYAYK*
Setaria italica PPO1 V328M (SEQ ID NO: 250)
AAVAGGAAEAPASAGARVSADCVVVGGGISGLCTAQALATKHGVGDVLVTEARARPGGNI
TTVERPDEGYLWEEGPNSFQPSDPVLTMAVDSGLKDDLVFGDPNAPRFVLWEGKLRPVPS
KPADLPFFDLMSIPGKLRAGFGALGIRPPPPGREESVEEFVRRNLGAEVFERLIEPFCSGVY
AGDPSKLSMKAAFGKVWRLEEAGGSIIGGTIKTIQERGKNPKPPRDPRLPTPKGQTVASFR
KGLAMLPNAITSSLGSKVKLSWKLTSITKSDGMGYVLVYETPEGVVSVQAKSVIMTIPSYVA
SDILRPLSSDAADALSRFYYPPMAAVTISYPKEAIRKECLIDGELQGFGQLHPRSQGVETLGT
IYSSSLFPNRAPAGRVLLLNYIGGATNTGIVSKSASELVEAVDRDLRKMLINPSAVDPLVLGV
RVWPQAIPQFLVGHLDLLEAAKSSLDRGGYDGLFLGGNYVAGVALGRCVEGAYESASQIS
DFLTKYAYK*
Setaria italica PPO1 G367A (SEQ ID NO: 251)
AAVAGGAAEAPASAGARVSADCVVVGGGISGLCTAQALATKHGVGDVLVTEARARPGGNI
TTVERPDEGYLWEEGPNSFQPSDPVLTMAVDSGLKDDLVFGDPNAPRFVLWEGKLRPVPS
KPADLPFFDLMSIPGKLRAGFGALGIRPPPPGREESVEEFVRRNLGAEVFERLIEPFCSGVY
AGDPSKLSMKAAFGKVWRLEEAGGSIIGGTIKTIQERGKNPKPPRDPRLPTPKGQTVASFR
KGLAMLPNAITSSLGSKVKLSWKLTSITKSDGMGYVLVYETPEGVVSVQAKSVIMTIPSYVA
SDILRPLSSDAADALSRFYYPPVAAVTISYPKEAIRKECLIDGELQGFGQLHPRSQGVETLATI
YSSSLFPNRAPAGRVLLLNYIGGATNTGIVSKSASELVEAVDRDLRKMLINPSAVDPLVLGV
RVWPQAIPQFLVGHLDLLEAAKSSLDRGGYDGLFLGGNYVAGVALGRCVEGAYESASQIS
DFLTKYAYK*
Setaria italica PPO1 P376G (SEQ ID NO: 252)
AAVAGGAAEAPASAGARVSADCVVVGGGISGLCTAQALATKHGVGDVLVTEARARPGGNI
TTVERPDEGYLWEEGPNSFQPSDPVLTMAVDSGLKDDLVFGDPNAPRFVLWEGKLRPVPS
KPADLPFFDLMSIPGKLRAGFGALGIRPPPPGREESVEEFVRRNLGAEVFERLIEPFCSGVY
AGDPSKLSMKAAFGKVWRLEEAGGSIIGGTIKTIQERGKNPKPPRDPRLPTPKGQTVASFR
KGLAMLPNAITSSLGSKVKLSWKLTSITKSDGMGYVLVYETPEGVVSVQAKSVIMTIPSYVA
SDILRPLSSDAADALSRFYYPPVAAVTISYPKEAIRKECLIDGELQGFGQLHPRSQGVETLGT
IYSSSLFGNRAPAGRVLLLNYIGGATNTGIVSKSASELVEAVDRDLRKMLINPSAVDPLVLGV
RVWPQAIPQFLVGHLDLLEAAKSSLDRGGYDGLFLGGNYVAGVALGRCVEGAYESASQIS
DFLTKYAYK*
Setaria italica PPO1 A381M (SEQ ID NO: 253)
AAVAGGAAEAPASAGARVSADCVVVGGGISGLCTAQALATKHGVGDVLVTEARARPGGNI
TTVERPDEGYLWEEGPNSFQPSDPVLTMAVDSGLKDDLVFGDPNAPRFVLWEGKLRPVPS
KPADLPFFDLMSIPGKLRAGFGALGIRPPPPGREESVEEFVRRNLGAEVFERLIEPFCSGVY
AGDPSKLSMKAAFGKVWRLEEAGGSIIGGTIKTIQERGKNPKPPRDPRLPTPKGQTVASFR
KGLAMLPNAITSSLGSKVKLSWKLTSITKSDGMGYVLVYETPEGVVSVQAKSVIMTIPSYVA
SDILRPLSSDAADALSRFYYPPVAAVTISYPKEAIRKECLIDGELQGFGQLHPRSQGVETLGT
IYSSSLFPNRAPMGRVLLLNYIGGATNTGIVSKSASELVEAVDRDLRKMLINPSAVDPLVLGV
RVWPQAIPQFLVGHLDLLEAAKSSLDRGGYDGLFLGGNYVAGVALGRCVEGAYESASQIS
DFLTKYAYK*
Setaria italica PPO1 L387V (SEQ ID NO: 254)
AAVAGGAAEAPASAGARVSADCVVVGGGISGLCTAQALATKHGVGDVLVTEARARPGGNI
TTVERPDEGYLWEEGPNSFQPSDPVLTMAVDSGLKDDLVFGDPNAPRFVLWEGKLRPVPS
KPADLPFFDLMSIPGKLRAGFGALGIRPPPPGREESVEEFVRRNLGAEVFERLIEPFCSGVY
AGDPSKLSMKAAFGKVWRLEEAGGSIIGGTIKTIQERGKNPKPPRDPRLPTPKGQTVASFR
KGLAMLPNAITSSLGSKVKLSWKLTSITKSDGMGYVLVYETPEGVVSVQAKSVIMTIPSYVA
SDILRPLSSDAADALSRFYYPPVAAVTISYPKEAIRKECLIDGELQGFGQLHPRSQGVETLGT
IYSSSLFPNRAPAGRVLLVNYIGGATNTGIVSKSASELVEAVDRDLRKMLINPSAVDPLVLGV
RVWPQAIPQFLVGHLDLLEAAKSSLDRGGYDGLFLGGNYVAGVALGRCVEGAYESASQIS
DFLTKYAYK*
Setaria italica PPO1 Y389C (SEQ ID NO: 255)
AAVAGGAAEAPASAGARVSADCVVVGGGISGLCTAQALATKHGVGDVLVTEARARPGGNI
TTVERPDEGYLWEEGPNSFQPSDPVLTMAVDSGLKDDLVFGDPNAPRFVLWEGKLRPVPS
KPADLPFFDLMSIPGKLRAGFGALGIRPPPPGREESVEEFVRRNLGAEVFERLIEPFCSGVY
AGDPSKLSMKAAFGKVWRLEEAGGSIIGGTIKTIQERGKNPKPPRDPRLPTPKGQTVASFR
KGLAMLPNAITSSLGSKVKLSWKLTSITKSDGMGYVLVYETPEGVVSVQAKSVIMTIPSYVA
SDILRPLSSDAADALSRFYYPPVAAVTISYPKEAIRKECLIDGELQGFGQLHPRSQGVETLGT
IYSSSLFPNRAPAGRVLLLNCIGGATNTGIVSKSASELVEAVDRDLRKMLINPSAVDPLVLGV
RVWPQAIPQFLVGHLDLLEAAKSSLDRGGYDGLFLGGNYVAGVALGRCVEGAYESASQIS
DFLTKYAYK*
Setaria italica PPO1 Y389L (SEQ ID NO: 256)
AAVAGGAAEAPASAGARVSADCVVVGGGISGLCTAQALATKHGVGDVLVTEARARPGGNI
TTVERPDEGYLWEEGPNSFQPSDPVLTMAVDSGLKDDLVFGDPNAPRFVLWEGKLRPVPS
KPADLPFFDLMSIPGKLRAGFGALGIRPPPPGREESVEEFVRRNLGAEVFERLIEPFCSGVY
AGDPSKLSMKAAFGKVWRLEEAGGSIIGGTIKTIQERGKNPKPPRDPRLPTPKGQTVASFR
KGLAMLPNAITSSLGSKVKLSWKLTSITKSDGMGYVLVYETPEGVVSVQAKSVIMTIPSYVA
SDILRPLSSDAADALSRFYYPPVAAVTISYPKEAIRKECLIDGELQGFGQLHPRSQGVETLGT
IYSSSLFPNRAPAGRVLLLNLIGGATNTGIVSKSASELVEAVDRDLRKMLINPSAVDPLVLGV
RVWPQAIPQFLVGHLDLLEAAKSSLDRGGYDGLFLGGNYVAGVALGRCVEGAYESASQIS
DFLTKYAYK*
Setaria italica PPO1 Y389M (SEQ ID NO: 257)
AAVAGGAAEAPASAGARVSADCVVVGGGISGLCTAQALATKHGVGDVLVTEARARPGGNI
TTVERPDEGYLWEEGPNSFQPSDPVLTMAVDSGLKDDLVFGDPNAPRFVLWEGKLRPVPS
KPADLPFFDLMSIPGKLRAGFGALGIRPPPPGREESVEEFVRRNLGAEVFERLIEPFCSGVY
AGDPSKLSMKAAFGKVWRLEEAGGSIIGGTIKTIQERGKNPKPPRDPRLPTPKGQTVASFR
KGLAMLPNAITSSLGSKVKLSWKLTSITKSDGMGYVLVYETPEGVVSVQAKSVIMTIPSYVA
SDILRPLSSDAADALSRFYYPPVAAVTISYPKEAIRKECLIDGELQGFGQLHPRSQGVETLGT
IYSSSLFPNRAPAGRVLLLNMIGGATNTGIVSKSASELVEAVDRDLRKMLINPSAVDPLVLGV
RVWPQAIPQFLVGHLDLLEAAKSSLDRGGYDGLFLGGNYVAGVALGRCVEGAYESASQIS
DFLTKYAYK*
Setaria italica PPO1 Y389T (SEQ ID NO: 258)
AAVAGGAAEAPASAGARVSADCVVVGGGISGLCTAQALATKHGVGDVLVTEARARPGGNI
TTVERPDEGYLWEEGPNSFQPSDPVLTMAVDSGLKDDLVFGDPNAPRFVLWEGKLRPVPS
KPADLPFFDLMSIPGKLRAGFGALGIRPPPPGREESVEEFVRRNLGAEVFERLIEPFCSGVY
AGDPSKLSMKAAFGKVWRLEEAGGSIIGGTIKTIQERGKNPKPPRDPRLPTPKGQTVASFR
KGLAMLPNAITSSLGSKVKLSWKLTSITKSDGMGYVLVYETPEGVVSVQAKSVIMTIPSYVA
SDILRPLSSDAADALSRFYYPPVAAVTISYPKEAIRKECLIDGELQGFGQLHPRSQGVETLGT
IYSSSLFPNRAPAGRVLLLNTIGGATNTGIVSKSASELVEAVDRDLRKMLINPSAVDPLVLGV
RVWPQAIPQFLVGHLDLLEAAKSSLDRGGYDGLFLGGNYVAGVALGRCVEGAYESASQIS
DFLTKYAYK*
Setaria italica PPO1 Y389V (SEQ ID NO: 259)
AAVAGGAAEAPASAGARVSADCVVVGGGISGLCTAQALATKHGVGDVLVTEARARPGGNI
TTVERPDEGYLWEEGPNSFQPSDPVLTMAVDSGLKDDLVFGDPNAPRFVLWEGKLRPVPS
KPADLPFFDLMSIPGKLRAGFGALGIRPPPPGREESVEEFVRRNLGAEVFERLIEPFCSGVY
AGDPSKLSMKAAFGKVWRLEEAGGSIIGGTIKTIQERGKNPKPPRDPRLPTPKGQTVASFR
KGLAMLPNAITSSLGSKVKLSWKLTSITKSDGMGYVLVYETPEGVVSVQAKSVIMTIPSYVA
SDILRPLSSDAADALSRFYYPPVAAVTISYPKEAIRKECLIDGELQGFGQLHPRSQGVETLGT
IYSSSLFPNRAPAGRVLLLNVIGGATNTGIVSKSASELVEAVDRDLRKMLINPSAVDPLVLGV
RVWPQAIPQFLVGHLDLLEAAKSSLDRGGYDGLFLGGNYVAGVALGRCVEGAYESASQIS
DFLTKYAYK*
Setaria italica PPO1 T394R (SEQ ID NO: 260)
AAVAGGAAEAPASAGARVSADCVVVGGGISGLCTAQALATKHGVGDVLVTEARARPGGNI
TTVERPDEGYLWEEGPNSFQPSDPVLTMAVDSGLKDDLVFGDPNAPRFVLWEGKLRPVPS
KPADLPFFDLMSIPGKLRAGFGALGIRPPPPGREESVEEFVRRNLGAEVFERLIEPFCSGVY
AGDPSKLSMKAAFGKVWRLEEAGGSIIGGTIKTIQERGKNPKPPRDPRLPTPKGQTVASFR
KGLAMLPNAITSSLGSKVKLSWKLTSITKSDGMGYVLVYETPEGVVSVQAKSVIMTIPSYVA
SDILRPLSSDAADALSRFYYPPVAAVTISYPKEAIRKECLIDGELQGFGQLHPRSQGVETLGT
IYSSSLFPNRAPAGRVLLLNYIGGARNTGIVSKSASELVEAVDRDLRKMLINPSAVDPLVLGV
RVWPQAIPQFLVGHLDLLEAAKSSLDRGGYDGLFLGGNYVAGVALGRCVEGAYESASQIS
DFLTKYAYK*
Setaria italica PPO1 V410C (SEQ ID NO: 261)
AAVAGGAAEAPASAGARVSADCVVVGGGISGLCTAQALATKHGVGDVLVTEARARPGGNI
TTVERPDEGYLWEEGPNSFQPSDPVLTMAVDSGLKDDLVFGDPNAPRFVLWEGKLRPVPS
KPADLPFFDLMSIPGKLRAGFGALGIRPPPPGREESVEEFVRRNLGAEVFERLIEPFCSGVY
AGDPSKLSMKAAFGKVWRLEEAGGSIIGGTIKTIQERGKNPKPPRDPRLPTPKGQTVASFR
KGLAMLPNAITSSLGSKVKLSWKLTSITKSDGMGYVLVYETPEGVVSVQAKSVIMTIPSYVA
SDILRPLSSDAADALSRFYYPPVAAVTISYPKEAIRKECLIDGELQGFGQLHPRSQGVETLGT
IYSSSLFPNRAPAGRVLLLNYIGGATNTGIVSKSASELVEACDRDLRKMLINPSAVDPLVLGV
RVWPQAIPQFLVGHLDLLEAAKSSLDRGGYDGLFLGGNYVAGVALGRCVEGAYESASQIS
DFLTKYAYK*
Setaria italica PPO1 V410I (SEQ ID NO: 262)
AAVAGGAAEAPASAGARVSADCVVVGGGISGLCTAQALATKHGVGDVLVTEARARPGGNI
TTVERPDEGYLWEEGPNSFQPSDPVLTMAVDSGLKDDLVFGDPNAPRFVLWEGKLRPVPS
KPADLPFFDLMSIPGKLRAGFGALGIRPPPPGREESVEEFVRRNLGAEVFERLIEPFCSGVY
AGDPSKLSMKAAFGKVWRLEEAGGSIIGGTIKTIQERGKNPKPPRDPRLPTPKGQTVASFR
KGLAMLPNAITSSLGSKVKLSWKLTSITKSDGMGYVLVYETPEGVVSVQAKSVIMTIPSYVA
SDILRPLSSDAADALSRFYYPPVAAVTISYPKEAIRKECLIDGELQGFGQLHPRSQGVETLGT
IYSSSLFPNRAPAGRVLLLNYIGGATNTGIVSKSASELVEAIDRDLRKMLINPSAVDPLVLGVR
VWPQAIPQFLVGHLDLLEAAKSSLDRGGYDGLFLGGNYVAGVALGRCVEGAYESASQISDF
LTKYAYK*
Setaria italica PPO1 D411H (SEQ ID NO: 263)
AAVAGGAAEAPASAGARVSADCVVVGGGISGLCTAQALATKHGVGDVLVTEARARPGGNI
TTVERPDEGYLWEEGPNSFQPSDPVLTMAVDSGLKDDLVFGDPNAPRFVLWEGKLRPVPS
KPADLPFFDLMSIPGKLRAGFGALGIRPPPPGREESVEEFVRRNLGAEVFERLIEPFCSGVY
AGDPSKLSMKAAFGKVWRLEEAGGSIIGGTIKTIQERGKNPKPPRDPRLPTPKGQTVASFR
KGLAMLPNAITSSLGSKVKLSWKLTSITKSDGMGYVLVYETPEGVVSVQAKSVIMTIPSYVA
SDILRPLSSDAADALSRFYYPPVAAVTISYPKEAIRKECLIDGELQGFGQLHPRSQGVETLGT
IYSSSLFPNRAPAGRVLLLNYIGGATNTGIVSKSASELVEAVHRDLRKMLINPSAVDPLVLGV
RVWPQAIPQFLVGHLDLLEAAKSSLDRGGYDGLFLGGNYVAGVALGRCVEGAYESASQIS
DFLTKYAYK*
Setaria italica PPO1 D411L (SEQ ID NO: 264)
AAVAGGAAEAPASAGARVSADCVVVGGGISGLCTAQALATKHGVGDVLVTEARARPGGNI
TTVERPDEGYLWEEGPNSFQPSDPVLTMAVDSGLKDDLVFGDPNAPRFVLWEGKLRPVPS
KPADLPFFDLMSIPGKLRAGFGALGIRPPPPGREESVEEFVRRNLGAEVFERLIEPFCSGVY
AGDPSKLSMKAAFGKVWRLEEAGGSIIGGTIKTIQERGKNPKPPRDPRLPTPKGQTVASFR
KGLAMLPNAITSSLGSKVKLSWKLTSITKSDGMGYVLVYETPEGVVSVQAKSVIMTIPSYVA
SDILRPLSSDAADALSRFYYPPVAAVTISYPKEAIRKECLIDGELQGFGQLHPRSQGVETLGT
IYSSSLFPNRAPAGRVLLLNYIGGATNTGIVSKSASELVEAVLRDLRKMLINPSAVDPLVLGV
RVWPQAIPQFLVGHLDLLEAAKSSLDRGGYDGLFLGGNYVAGVALGRCVEGAYESASQIS
DFLTKYAYK*
Setaria italica PPO1 D411N (SEQ ID NO: 265)
AAVAGGAAEAPASAGARVSADCVVVGGGISGLCTAQALATKHGVGDVLVTEARARPGGNI
TTVERPDEGYLWEEGPNSFQPSDPVLTMAVDSGLKDDLVFGDPNAPRFVLWEGKLRPVPS
KPADLPFFDLMSIPGKLRAGFGALGIRPPPPGREESVEEFVRRNLGAEVFERLIEPFCSGVY
AGDPSKLSMKAAFGKVWRLEEAGGSIIGGTIKTIQERGKNPKPPRDPRLPTPKGQTVASFR
KGLAMLPNAITSSLGSKVKLSWKLTSITKSDGMGYVLVYETPEGVVSVQAKSVIMTIPSYVA
SDILRPLSSDAADALSRFYYPPVAAVTISYPKEAIRKECLIDGELQGFGQLHPRSQGVETLGT
IYSSSLFPNRAPAGRVLLLNYIGGATNTGIVSKSASELVEAVNRDLRKMLINPSAVDPLVLGV
RVWPQAIPQFLVGHLDLLEAAKSSLDRGGYDGLFLGGNYVAGVALGRCVEGAYESASQIS
DFLTKYAYK*
Setaria italica PPO1 R412K (SEQ ID NO: 266)
AAVAGGAAEAPASAGARVSADCVVVGGGISGLCTAQALATKHGVGDVLVTEARARPGGNI
TTVERPDEGYLWEEGPNSFQPSDPVLTMAVDSGLKDDLVFGDPNAPRFVLWEGKLRPVPS
KPADLPFFDLMSIPGKLRAGFGALGIRPPPPGREESVEEFVRRNLGAEVFERLIEPFCSGVY
AGDPSKLSMKAAFGKVWRLEEAGGSIIGGTIKTIQERGKNPKPPRDPRLPTPKGQTVASFR
KGLAMLPNAITSSLGSKVKLSWKLTSITKSDGMGYVLVYETPEGVVSVQAKSVIMTIPSYVA
SDILRPLSSDAADALSRFYYPPVAAVTISYPKEAIRKECLIDGELQGFGQLHPRSQGVETLGT
IYSSSLFPNRAPAGRVLLLNYIGGATNTGIVSKSASELVEAVDKDLRKMLINPSAVDPLVLGV
RVWPQAIPQFLVGHLDLLEAAKSSLDRGGYDGLFLGGNYVAGVALGRCVEGAYESASQIS
DFLTKYAYK*
Setaria italica PPO1 V424Q (SEQ ID NO: 267)
AAVAGGAAEAPASAGARVSADCVVVGGGISGLCTAQALATKHGVGDVLVTEARARPGGNI
TTVERPDEGYLWEEGPNSFQPSDPVLTMAVDSGLKDDLVFGDPNAPRFVLWEGKLRPVPS
KPADLPFFDLMSIPGKLRAGFGALGIRPPPPGREESVEEFVRRNLGAEVFERLIEPFCSGVY
AGDPSKLSMKAAFGKVWRLEEAGGSIIGGTIKTIQERGKNPKPPRDPRLPTPKGQTVASFR
KGLAMLPNAITSSLGSKVKLSWKLTSITKSDGMGYVLVYETPEGVVSVQAKSVIMTIPSYVA
SDILRPLSSDAADALSRFYYPPVAAVTISYPKEAIRKECLIDGELQGFGQLHPRSQGVETLGT
IYSSSLFPNRAPAGRVLLLNYIGGATNTGIVSKSASELVEAVDRDLRKMLINPSAQDPLVLGV
RVWPQAIPQFLVGHLDLLEAAKSSLDRGGYDGLFLGGNYVAGVALGRCVEGAYESASQIS
DFLTKYAYK*
Setaria italica PPO1 F441R (SEQ ID NO: 268)
AAVAGGAAEAPASAGARVSADCVVVGGGISGLCTAQALATKHGVGDVLVTEARARPGGNI
TTVERPDEGYLWEEGPNSFQPSDPVLTMAVDSGLKDDLVFGDPNAPRFVLWEGKLRPVPS
KPADLPFFDLMSIPGKLRAGFGALGIRPPPPGREESVEEFVRRNLGAEVFERLIEPFCSGVY
AGDPSKLSMKAAFGKVWRLEEAGGSIIGGTIKTIQERGKNPKPPRDPRLPTPKGQTVASFR
KGLAMLPNAITSSLGSKVKLSWKLTSITKSDGMGYVLVYETPEGVVSVQAKSVIMTIPSYVA
SDILRPLSSDAADALSRFYYPPVAAVTISYPKEAIRKECLIDGELQGFGQLHPRSQGVETLGT
IYSSSLFPNRAPAGRVLLLNYIGGATNTGIVSKSASELVEAVDRDLRKMLINPSAVDPLVLGV
RVWPQAIPQRLVGHLDLLEAAKSSLDRGGYDGLFLGGNYVAGVALGRCVEGAYESASQIS
DFLTKYAYK*
Setaria italica PPO1 L442M (SEQ ID NO: 269)
AAVAGGAAEAPASAGARVSADCVVVGGGISGLCTAQALATKHGVGDVLVTEARARPGGNI
TTVERPDEGYLWEEGPNSFQPSDPVLTMAVDSGLKDDLVFGDPNAPRFVLWEGKLRPVPS
KPADLPFFDLMSIPGKLRAGFGALGIRPPPPGREESVEEFVRRNLGAEVFERLIEPFCSGVY
AGDPSKLSMKAAFGKVWRLEEAGGSIIGGTIKTIQERGKNPKPPRDPRLPTPKGQTVASFR
KGLAMLPNAITSSLGSKVKLSWKLTSITKSDGMGYVLVYETPEGVVSVQAKSVIMTIPSYVA
SDILRPLSSDAADALSRFYYPPVAAVTISYPKEAIRKECLIDGELQGFGQLHPRSQGVETLGT
IYSSSLFPNRAPAGRVLLLNYIGGATNTGIVSKSASELVEAVDRDLRKMLINPSAVDPLVLGV
RVWPQAIPQFMVGHLDLLEAAKSSLDRGGYDGLFLGGNYVAGVALGRCVEGAYESASQIS
DFLTKYAYK*
Setaria italica PPO1 L442N (SEQ ID NO: 270)
AAVAGGAAEAPASAGARVSADCVVVGGGISGLCTAQALATKHGVGDVLVTEARARPGGNI
TTVERPDEGYLWEEGPNSFQPSDPVLTMAVDSGLKDDLVFGDPNAPRFVLWEGKLRPVPS
KPADLPFFDLMSIPGKLRAGFGALGIRPPPPGREESVEEFVRRNLGAEVFERLIEPFCSGVY
AGDPSKLSMKAAFGKVWRLEEAGGSIIGGTIKTIQERGKNPKPPRDPRLPTPKGQTVASFR
KGLAMLPNAITSSLGSKVKLSWKLTSITKSDGMGYVLVYETPEGVVSVQAKSVIMTIPSYVA
SDILRPLSSDAADALSRFYYPPVAAVTISYPKEAIRKECLIDGELQGFGQLHPRSQGVETLGT
IYSSSLFPNRAPAGRVLLLNYIGGATNTGIVSKSASELVEAVDRDLRKMLINPSAVDPLVLGV
RVWPQAIPQFNVGHLDLLEAAKSSLDRGGYDGLFLGGNYVAGVALGRCVEGAYESASQIS
DFLTKYAYK*
Setaria italica PPO1 G473N (SEQ ID NO: 271)
AAVAGGAAEAPASAGARVSADCVVVGGGISGLCTAQALATKHGVGDVLVTEARARPGGNI
TTVERPDEGYLWEEGPNSFQPSDPVLTMAVDSGLKDDLVFGDPNAPRFVLWEGKLRPVPS
KPADLPFFDLMSIPGKLRAGFGALGIRPPPPGREESVEEFVRRNLGAEVFERLIEPFCSGVY
AGDPSKLSMKAAFGKVWRLEEAGGSIIGGTIKTIQERGKNPKPPRDPRLPTPKGQTVASFR
KGLAMLPNAITSSLGSKVKLSWKLTSITKSDGMGYVLVYETPEGVVSVQAKSVIMTIPSYVA
SDILRPLSSDAADALSRFYYPPVAAVTISYPKEAIRKECLIDGELQGFGQLHPRSQGVETLGT
IYSSSLFPNRAPAGRVLLLNYIGGATNTGIVSKSASELVEAVDRDLRKMLINPSAVDPLVLGV
RVWPQAIPQFLVGHLDLLEAAKSSLDRGGYDGLFLGGNYVANVALGRCVEGAYESASQISD
FLTKYAYK*
Setaria italica PPO1 V474L (SEQ ID NO: 272)
AAVAGGAAEAPASAGARVSADCVVVGGGISGLCTAQALATKHGVGDVLVTEARARPGGNI
TTVERPDEGYLWEEGPNSFQPSDPVLTMAVDSGLKDDLVFGDPNAPRFVLWEGKLRPVPS
KPADLPFFDLMSIPGKLRAGFGALGIRPPPPGREESVEEFVRRNLGAEVFERLIEPFCSGVY
AGDPSKLSMKAAFGKVWRLEEAGGSIIGGTIKTIQERGKNPKPPRDPRLPTPKGQTVASFR
KGLAMLPNAITSSLGSKVKLSWKLTSITKSDGMGYVLVYETPEGVVSVQAKSVIMTIPSYVA
SDILRPLSSDAADALSRFYYPPVAAVTISYPKEAIRKECLIDGELQGFGQLHPRSQGVETLGT
IYSSSLFPNRAPAGRVLLLNYIGGATNTGIVSKSASELVEAVDRDLRKMLINPSAVDPLVLGV
RVWPQAIPQFLVGHLDLLEAAKSSLDRGGYDGLFLGGNYVAGLALGRCVEGAYESASQISD
FLTKYAYK*
Setaria italica PPO1 V474N (SEQ ID NO: 273)
AAVAGGAAEAPASAGARVSADCVVVGGGISGLCTAQALATKHGVGDVLVTEARARPGGNI
TTVERPDEGYLWEEGPNSFQPSDPVLTMAVDSGLKDDLVFGDPNAPRFVLWEGKLRPVPS
KPADLPFFDLMSIPGKLRAGFGALGIRPPPPGREESVEEFVRRNLGAEVFERLIEPFCSGVY
AGDPSKLSMKAAFGKVWRLEEAGGSIIGGTIKTIQERGKNPKPPRDPRLPTPKGQTVASFR
KGLAMLPNAITSSLGSKVKLSWKLTSITKSDGMGYVLVYETPEGVVSVQAKSVIMTIPSYVA
SDILRPLSSDAADALSRFYYPPVAAVTISYPKEAIRKECLIDGELQGFGQLHPRSQGVETLGT
IYSSSLFPNRAPAGRVLLLNYIGGATNTGIVSKSASELVEAVDRDLRKMLINPSAVDPLVLGV
RVWPQAIPQFLVGHLDLLEAAKSSLDRGGYDGLFLGGNYVAGNALGRCVEGAYESASQIS
DFLTKYAYK*
Setaria italica PPO1 C479Q (SEQ ID NO: 274)
AAVAGGAAEAPASAGARVSADCVVVGGGISGLCTAQALATKHGVGDVLVTEARARPGGNI
TTVERPDEGYLWEEGPNSFQPSDPVLTMAVDSGLKDDLVFGDPNAPRFVLWEGKLRPVPS
KPADLPFFDLMSIPGKLRAGFGALGIRPPPPGREESVEEFVRRNLGAEVFERLIEPFCSGVY
AGDPSKLSMKAAFGKVWRLEEAGGSIIGGTIKTIQERGKNPKPPRDPRLPTPKGQTVASFR
KGLAMLPNAITSSLGSKVKLSWKLTSITKSDGMGYVLVYETPEGVVSVQAKSVIMTIPSYVA
SDILRPLSSDAADALSRFYYPPVAAVTISYPKEAIRKECLIDGELQGFGQLHPRSQGVETLGT
IYSSSLFPNRAPAGRVLLLNYIGGATNTGIVSKSASELVEAVDRDLRKMLINPSAVDPLVLGV
RVWPQAIPQFLVGHLDLLEAAKSSLDRGGYDGLFLGGNYVAGVALGRQVEGAYESASQIS
DFLTKYAYK*
Arabidopsis thaliana PPO1 S271L Y392M (SEQ ID NO: 275)
SVAGGPTVGSSKIEGGGGTTITTDCVIVGGGISGLCIAQALATKHPDAAPNLIVTEAKDRVGG
NIITREENGFLWEEGPNSFQPSDPMLTMVVDSGLKDDLVLGDPTAPRFVLWNGKLRPVPSK
LTDLPFFDLMSIGGKIRAGFGALGIRPSPPGREESVEEFVRRNLGDEVFERLIEPFCSGVYA
GDPSKLSMKAAFGKVWKLEQNGGSIIGGTFKAIQERKNAPKAERDPRLPKPQGQTVGSFR
KGLRMLPEAISARLGSKVKLSWKLLGITKLESGGYNLTYETPDGLVSVQSKSVVMTVPSHVA
SGLLRPLSESAANALSKLYYPPVAAVSISYPKEAIRTECLIDGELKGFGQLHPRTQGVETLGT
IYSSSLFPNRAPPGRILLLNMIGGSTNTGILSKSEGELVEAVDRDLRKMLIKPNSTDPLKLGV
RVWPQAIPQFLVGHFDILDTAKSSLTSSGYEGLFLGGNYVAGVALGRCVEGAYETAIEVNNF
MSRYAYK
Arabidopsis thaliana PPO1 S271L Y392V (SEQ ID NO: 276)
SVAGGPTVGSSKIEGGGGTTITTDCVIVGGGISGLCIAQALATKHPDAAPNLIVTEAKDRVGG
NIITREENGFLWEEGPNSFQPSDPMLTMVVDSGLKDDLVLGDPTAPRFVLWNGKLRPVPSK
LTDLPFFDLMSIGGKIRAGFGALGIRPSPPGREESVEEFVRRNLGDEVFERLIEPFCSGVYA
GDPSKLSMKAAFGKVWKLEQNGGSIIGGTFKAIQERKNAPKAERDPRLPKPQGQTVGSFR
KGLRMLPEAISARLGSKVKLSWKLLGITKLESGGYNLTYETPDGLVSVQSKSVVMTVPSHVA
SGLLRPLSESAANALSKLYYPPVAAVSISYPKEAIRTECLIDGELKGFGQLHPRTQGVETLGT
IYSSSLFPNRAPPGRILLLNVIGGSTNTGILSKSEGELVEAVDRDLRKMLIKPNSTDPLKLGVR
VWPQAIPQFLVGHFDILDTAKSSLTSSGYEGLFLGGNYVAGVALGRCVEGAYETAIEVNNF
MSRYAYK*
Arabidopsis thaliana PPO1 V331L Y392V L445N (SEQ ID NO: 277)
SVAGGPTVGSSKIEGGGGTTITTDCVIVGGGISGLCIAQALATKHPDAAPNLIVTEAKDRVGG
NIITREENGFLWEEGPNSFQPSDPMLTMVVDSGLKDDLVLGDPTAPRFVLWNGKLRPVPSK
LTDLPFFDLMSIGGKIRAGFGALGIRPSPPGREESVEEFVRRNLGDEVFERLIEPFCSGVYA
GDPSKLSMKAAFGKVWKLEQNGGSIIGGTFKAIQERKNAPKAERDPRLPKPQGQTVGSFR
KGLRMLPEAISARLGSKVKLSWKLSGITKLESGGYNLTYETPDGLVSVQSKSVVMTVPSHV
ASGLLRPLSESAANALSKLYYPPLAAVSISYPKEAIRTECLIDGELKGFGQLHPRTQGVETLG
TIYSSSLFPNRAPPGRILLLNVIGGSTNTGILSKSEGELVEAVDRDLRKMLIKPNSTDPLKLGV
RVWPQAIPQFNVGHFDILDTAKSSLTSSGYEGLFLGGNYVAGVALGRCVEGAYETAIEVNN
FMSRYAYK*
Arabidopsis thaliana PPO1 S271L G370A Y392V T397R L445N (SEQ ID NO: 278)
SVAGGPTVGSSKIEGGGGTTITTDCVIVGGGISGLCIAQALATKHPDAAPNLIVTEAKDRVGG
NIITREENGFLWEEGPNSFQPSDPMLTMVVDSGLKDDLVLGDPTAPRFVLWNGKLRPVPSK
LTDLPFFDLMSIGGKIRAGFGALGIRPSPPGREESVEEFVRRNLGDEVFERLIEPFCSGVYA
GDPSKLSMKAAFGKVWKLEQNGGSIIGGTFKAIQERKNAPKAERDPRLPKPQGQTVGSFR
KGLRMLPEAISARLGSKVKLSWKLLGITKLESGGYNLTYETPDGLVSVQSKSVVMTVPSHVA
SGLLRPLSESAANALSKLYYPPVAAVSISYPKEAIRTECLIDGELKGFGQLHPRTQGVETLATI
YSSSLFPNRAPPGRILLLNVIGGSRNTGILSKSEGELVEAVDRDLRKMLIKPNSTDPLKLGVR
VWPQAIPQFNVGHFDILDTAKSSLTSSGYEGLFLGGNYVAGVALGRCVEGAYETAIEVNNF
MSRYAYK*
Arabidopsis thaliana PPO1 S271L V331M (SEQ ID NO: 279)
SVAGGPTVGSSKIEGGGGTTITTDCVIVGGGISGLCIAQALATKHPDAAPNLIVTEAKDRVGG
NIITREENGFLWEEGPNSFQPSDPMLTMVVDSGLKDDLVLGDPTAPRFVLWNGKLRPVPSK
LTDLPFFDLMSIGGKIRAGFGALGIRPSPPGREESVEEFVRRNLGDEVFERLIEPFCSGVYA
GDPSKLSMKAAFGKVWKLEQNGGSIIGGTFKAIQERKNAPKAERDPRLPKPQGQTVGSFR
KGLRMLPEAISARLGSKVKLSWKLLGITKLESGGYNLTYETPDGLVSVQSKSVVMTVPSHVA
SGLLRPLSESAANALSKLYYPPMAAVSISYPKEAIRTECLIDGELKGFGQLHPRTQGVETLGT
IYSSSLFPNRAPPGRILLLNYIGGSTNTGILSKSEGELVEAVDRDLRKMLIKPNSTDPLKLGVR
VWPQAIPQFLVGHFDILDTAKSSLTSSGYEGLFLGGNYVAGVALGRCVEGAYETAIEVNNF
MSRYAYK*
Arabidopsis thaliana PPO1 S271L Y328F G370A (SEQ ID NO: 280)
SVAGGPTVGSSKIEGGGGTTITTDCVIVGGGISGLCIAQALATKHPDAAPNLIVTEAKDRVGG
NIITREENGFLWEEGPNSFQPSDPMLTMVVDSGLKDDLVLGDPTAPRFVLWNGKLRPVPSK
LTDLPFFDLMSIGGKIRAGFGALGIRPSPPGREESVEEFVRRNLGDEVFERLIEPFCSGVYA
GDPSKLSMKAAFGKVWKLEQNGGSIIGGTFKAIQERKNAPKAERDPRLPKPQGQTVGSFR
KGLRMLPEAISARLGSKVKLSWKLLGITKLESGGYNLTYETPDGLVSVQSKSVVMTVPSHVA
SGLLRPLSESAANALSKLYFPPVAAVSISYPKEAIRTECLIDGELKGFGQLHPRTQGVETLATI
YSSSLFPNRAPPGRILLLNYIGGSTNTGILSKSEGELVEAVDRDLRKMLIKPNSTDPLKLGVR
VWPQAIPQFLVGHFDILDTAKSSLTSSGYEGLFLGGNYVAGVALGRCVEGAYETAIEVNNF
MSRYAYK*
Arabidopsis thaliana PPO1 S271L Y327T V331L T397R L445M (SEQ ID NO: 281)
SVAGGPTVGSSKIEGGGGTTITTDCVIVGGGISGLCIAQALATKHPDAAPNLIVTEAKDRVGG
NIITREENGFLWEEGPNSFQPSDPMLTMVVDSGLKDDLVLGDPTAPRFVLWNGKLRPVPSK
LTDLPFFDLMSIGGKIRAGFGALGIRPSPPGREESVEEFVRRNLGDEVFERLIEPFCSGVYA
GDPSKLSMKAAFGKVWKLEQNGGSIIGGTFKAIQERKNAPKAERDPRLPKPQGQTVGSFR
KGLRMLPEAISARLGSKVKLSWKLLGITKLESGGYNLTYETPDGLVSVQSKSVVMTVPSHVA
SGLLRPLSESAANALSKLTYPPLAAVSISYPKEAIRTECLIDGELKGFGQLHPRTQGVETLGTI
YSSSLFPNRAPPGRILLLNYIGGSRNTGILSKSEGELVEAVDRDLRKMLIKPNSTDPLKLGVR
VWPQAIPQFMVGHFDILDTAKSSLTSSGYEGLFLGGNYVAGVALGRCVEGAYETAIEVNNF
MSRYAYK*
Arabidopsis thaliana PPO1 S271L Y392V L445M (SEQ ID NO: 282)
SVAGGPTVGSSKIEGGGGTTITTDCVIVGGGISGLCIAQALATKHPDAAPNLIVTEAKDRVGG
NIITREENGFLWEEGPNSFQPSDPMLTMVVDSGLKDDLVLGDPTAPRFVLWNGKLRPVPSK
LTDLPFFDLMSIGGKIRAGFGALGIRPSPPGREESVEEFVRRNLGDEVFERLIEPFCSGVYA
GDPSKLSMKAAFGKVWKLEQNGGSIIGGTFKAIQERKNAPKAERDPRLPKPQGQTVGSFR
KGLRMLPEAISARLGSKVKLSWKLLGITKLESGGYNLTYETPDGLVSVQSKSVVMTVPSHVA
SGLLRPLSESAANALSKLYYPPVAAVSISYPKEAIRTECLIDGELKGFGQLHPRTQGVETLGT
IYSSSLFPNRAPPGRILLLNVIGGSTNTGILSKSEGELVEAVDRDLRKMLIKPNSTDPLKLGVR
VWPQAIPQFMVGHFDILDTAKSSLTSSGYEGLFLGGNYVAGVALGRCVEGAYETAIEVNNF
MSRYAYK*
Arabidopsis thaliana PPO1 Y392M T427Q L445M (SEQ ID NO: 283)
SVAGGPTVGSSKIEGGGGTTITTDCVIVGGGISGLCIAQALATKHPDAAPNLIVTEAKDRVGG
NIITREENGFLWEEGPNSFQPSDPMLTMVVDSGLKDDLVLGDPTAPRFVLWNGKLRPVPSK
LTDLPFFDLMSIGGKIRAGFGALGIRPSPPGREESVEEFVRRNLGDEVFERLIEPFCSGVYA
GDPSKLSMKAAFGKVWKLEQNGGSIIGGTFKAIQERKNAPKAERDPRLPKPQGQTVGSFR
KGLRMLPEAISARLGSKVKLSWKLSGITKLESGGYNLTYETPDGLVSVQSKSVVMTVPSHV
ASGLLRPLSESAANALSKLYYPPVAAVSISYPKEAIRTECLIDGELKGFGQLHPRTQGVETLG
TIYSSSLFPNRAPPGRILLLNMIGGSTNTGILSKSEGELVEAVDRDLRKMLIKPNSQDPLKLG
VRVWPQAIPQFMVGHFDILDTAKSSLTSSGYEGLFLGGNYVAGVALGRCVEGAYETAIEVN
NFMSRYAYK*
Arabidopsis thaliana PPO1 S271L Y327D V331M (SEQ ID NO: 284)
SVAGGPTVGSSKIEGGGGTTITTDCVIVGGGISGLCIAQALATKHPDAAPNLIVTEAKDRVGG
NIITREENGFLWEEGPNSFQPSDPMLTMVVDSGLKDDLVLGDPTAPRFVLWNGKLRPVPSK
LTDLPFFDLMSIGGKIRAGFGALGIRPSPPGREESVEEFVRRNLGDEVFERLIEPFCSGVYA
GDPSKLSMKAAFGKVWKLEQNGGSIIGGTFKAIQERKNAPKAERDPRLPKPQGQTVGSFR
KGLRMLPEAISARLGSKVKLSWKLLGITKLESGGYNLTYETPDGLVSVQSKSVVMTVPSHVA
SGLLRPLSESAANALSKLDYPPMAAVSISYPKEAIRTECLIDGELKGFGQLHPRTQGVETLG
TIYSSSLFPNRAPPGRILLLNYIGGSTNTGILSKSEGELVEAVDRDLRKMLIKPNSTDPLKLGV
RVWPQAIPQFLVGHFDILDTAKSSLTSSGYEGLFLGGNYVAGVALGRCVEGAYETAIEVNNF
MSRYAYK*
Arabidopsis thaliana PPO1 S271L Y327C Y328F Y392M L445M (SEQ ID NO: 285)
SVAGGPTVGSSKIEGGGGTTITTDCVIVGGGISGLCIAQALATKHPDAAPNLIVTEAKDRVGG
NIITREENGFLWEEGPNSFQPSDPMLTMVVDSGLKDDLVLGDPTAPRFVLWNGKLRPVPSK
LTDLPFFDLMSIGGKIRAGFGALGIRPSPPGREESVEEFVRRNLGDEVFERLIEPFCSGVYA
GDPSKLSMKAAFGKVWKLEQNGGSIIGGTFKAIQERKNAPKAERDPRLPKPQGQTVGSFR
KGLRMLPEAISARLGSKVKLSWKLLGITKLESGGYNLTYETPDGLVSVQSKSVVMTVPSHVA
SGLLRPLSESAANALSKLCFPPVAAVSISYPKEAIRTECLIDGELKGFGQLHPRTQGVETLGT
IYSSSLFPNRAPPGRILLLNMIGGSTNTGILSKSEGELVEAVDRDLRKMLIKPNSTDPLKLGV
RVWPQAIPQFMVGHFDILDTAKSSLTSSGYEGLFLGGNYVAGVALGRCVEGAYETAIEVNN
FMSRYAYK*
Arabidopsis thaliana PPO1 Y327D V331L (SEQ ID NO: 286)
SVAGGPTVGSSKIEGGGGTTITTDCVIVGGGISGLCIAQALATKHPDAAPNLIVTEAKDRVGG
NIITREENGFLWEEGPNSFQPSDPMLTMVVDSGLKDDLVLGDPTAPRFVLWNGKLRPVPSK
LTDLPFFDLMSIGGKIRAGFGALGIRPSPPGREESVEEFVRRNLGDEVFERLIEPFCSGVYA
GDPSKLSMKAAFGKVWKLEQNGGSIIGGTFKAIQERKNAPKAERDPRLPKPQGQTVGSFR
KGLRMLPEAISARLGSKVKLSWKLSGITKLESGGYNLTYETPDGLVSVQSKSVVMTVPSHV
ASGLLRPLSESAANALSKLDYPPLAAVSISYPKEAIRTECLIDGELKGFGQLHPRTQGVETLG
TIYSSSLFPNRAPPGRILLLNYIGGSTNTGILSKSEGELVEAVDRDLRKMLIKPNSTDPLKLGV
RVWPQAIPQFLVGHFDILDTAKSSLTSSGYEGLFLGGNYVAGVALGRCVEGAYETAIEVNNF
MSRYAYK*
Arabidopsis thaliana PPO1 V331L G370A L445N (SEQ ID NO: 287)
SVAGGPTVGSSKIEGGGGTTITTDCVIVGGGISGLCIAQALATKHPDAAPNLIVTEAKDRVGG
NIITREENGFLWEEGPNSFQPSDPMLTMVVDSGLKDDLVLGDPTAPRFVLWNGKLRPVPSK
LTDLPFFDLMSIGGKIRAGFGALGIRPSPPGREESVEEFVRRNLGDEVFERLIEPFCSGVYA
GDPSKLSMKAAFGKVWKLEQNGGSIIGGTFKAIQERKNAPKAERDPRLPKPQGQTVGSFR
KGLRMLPEAISARLGSKVKLSWKLSGITKLESGGYNLTYETPDGLVSVQSKSVVMTVPSHV
ASGLLRPLSESAANALSKLYYPPLAAVSISYPKEAIRTECLIDGELKGFGQLHPRTQGVETLA
TIYSSSLFPNRAPPGRILLLNYIGGSTNTGILSKSEGELVEAVDRDLRKMLIKPNSTDPLKLGV
RVWPQAIPQFNVGHFDILDTAKSSLTSSGYEGLFLGGNYVAGVALGRCVEGAYETAIEVNN
FMSRYAYK*
Arabidopsis thaliana PPO1 S271L G370A L445N (SEQ ID NO: 288)
SVAGGPTVGSSKIEGGGGTTITTDCVIVGGGISGLCIAQALATKHPDAAPNLIVTEAKDRVGG
NIITREENGFLWEEGPNSFQPSDPMLTMVVDSGLKDDLVLGDPTAPRFVLWNGKLRPVPSK
LTDLPFFDLMSIGGKIRAGFGALGIRPSPPGREESVEEFVRRNLGDEVFERLIEPFCSGVYA
GDPSKLSMKAAFGKVWKLEQNGGSIIGGTFKAIQERKNAPKAERDPRLPKPQGQTVGSFR
KGLRMLPEAISARLGSKVKLSWKLLGITKLESGGYNLTYETPDGLVSVQSKSVVMTVPSHVA
SGLLRPLSESAANALSKLYYPPVAAVSISYPKEAIRTECLIDGELKGFGQLHPRTQGVETLATI
YSSSLFPNRAPPGRILLLNYIGGSTNTGILSKSEGELVEAVDRDLRKMLIKPNSTDPLKLGVR
VWPQAIPQFNVGHFDILDTAKSSLTSSGYEGLFLGGNYVAGVALGRCVEGAYETAIEVNNF
MSRYAYK*
Setaria italica PPO1 T268L Y389M (SEQ ID NO: 289)
AAVAGGAAEAPASAGARVSADCVVVGGGISGLCTAQALATKHGVGDVLVTEARARPGGNI
TTVERPDEGYLWEEGPNSFQPSDPVLTMAVDSGLKDDLVFGDPNAPRFVLWEGKLRPVPS
KPADLPFFDLMSIPGKLRAGFGALGIRPPPPGREESVEEFVRRNLGAEVFERLIEPFCSGVY
AGDPSKLSMKAAFGKVWRLEEAGGSIIGGTIKTIQERGKNPKPPRDPRLPTPKGQTVASFR
KGLAMLPNAITSSLGSKVKLSWKLLSITKSDGMGYVLVYETPEGVVSVQAKSVIMTIPSYVAS
DILRPLSSDAADALSRFYYPPVAAVTISYPKEAIRKECLIDGELQGFGQLHPRSQGVETLGTI
YSSSLFPNRAPAGRVLLLNMIGGATNTGIVSKSASELVEAVDRDLRKMLINPSAVDPLVLGV
RVWPQAIPQFLVGHLDLLEAAKSSLDRGGYDGLFLGGNYVAGVALGRCVEGAYESASQIS
DFLTKYAYK*
Setaria italica PPO1 T268L Y389V (SEQ ID NO: 290)
AAVAGGAAEAPASAGARVSADCVVVGGGISGLCTAQALATKHGVGDVLVTEARARPGGNI
TTVERPDEGYLWEEGPNSFQPSDPVLTMAVDSGLKDDLVFGDPNAPRFVLWEGKLRPVPS
KPADLPFFDLMSIPGKLRAGFGALGIRPPPPGREESVEEFVRRNLGAEVFERLIEPFCSGVY
AGDPSKLSMKAAFGKVWRLEEAGGSIIGGTIKTIQERGKNPKPPRDPRLPTPKGQTVASFR
KGLAMLPNAITSSLGSKVKLSWKLLSITKSDGMGYVLVYETPEGVVSVQAKSVIMTIPSYVAS
DILRPLSSDAADALSRFYYPPVAAVTISYPKEAIRKECLIDGELQGFGQLHPRSQGVETLGTI
YSSSLFPNRAPAGRVLLLNVIGGATNTGIVSKSASELVEAVDRDLRKMLINPSAVDPLVLGV
RVWPQAIPQFLVGHLDLLEAAKSSLDRGGYDGLFLGGNYVAGVALGRCVEGAYESASQIS
DFLTKYAYK*
Setaria italica PPO1 V328L Y389V L442N (SEQ ID NO: 291)
AAVAGGAAEAPASAGARVSADCVVVGGGISGLCTAQALATKHGVGDVLVTEARARPGGNI
TTVERPDEGYLWEEGPNSFQPSDPVLTMAVDSGLKDDLVFGDPNAPRFVLWEGKLRPVPS
KPADLPFFDLMSIPGKLRAGFGALGIRPPPPGREESVEEFVRRNLGAEVFERLIEPFCSGVY
AGDPSKLSMKAAFGKVWRLEEAGGSIIGGTIKTIQERGKNPKPPRDPRLPTPKGQTVASFR
KGLAMLPNAITSSLGSKVKLSWKLTSITKSDGMGYVLVYETPEGVVSVQAKSVIMTIPSYVA
SDILRPLSSDAADALSRFYYPPLAAVTISYPKEAIRKECLIDGELQGFGQLHPRSQGVETLGTI
YSSSLFPNRAPAGRVLLLNVIGGATNTGIVSKSASELVEAVDRDLRKMLINPSAVDPLVLGV
RVWPQAIPQFNVGHLDLLEAAKSSLDRGGYDGLFLGGNYVAGVALGRCVEGAYESASQIS
DFLTKYAYK*
Setaria italica PPO1 T268L G367A Y389V T394R L442N (SEQ ID NO: 292)
AAVAGGAAEAPASAGARVSADCVVVGGGISGLCTAQALATKHGVGDVLVTEARARPGGNI
TTVERPDEGYLWEEGPNSFQPSDPVLTMAVDSGLKDDLVFGDPNAPRFVLWEGKLRPVPS
KPADLPFFDLMSIPGKLRAGFGALGIRPPPPGREESVEEFVRRNLGAEVFERLIEPFCSGVY
AGDPSKLSMKAAFGKVWRLEEAGGSIIGGTIKTIQERGKNPKPPRDPRLPTPKGQTVASFR
KGLAMLPNAITSSLGSKVKLSWKLLSITKSDGMGYVLVYETPEGVVSVQAKSVIMTIPSYVAS
DILRPLSSDAADALSRFYYPPVAAVTISYPKEAIRKECLIDGELQGFGQLHPRSQGVETLATIY
SSSLFPNRAPAGRVLLLNVIGGARNTGIVSKSASELVEAVDRDLRKMLINPSAVDPLVLGVR
VWPQAIPQFNVGHLDLLEAAKSSLDRGGYDGLFLGGNYVAGVALGRCVEGAYESASQISD
FLTKYAYK*
Setaria italica PPO1 T268L V328M (SEQ ID NO: 293)
AAVAGGAAEAPASAGARVSADCVVVGGGISGLCTAQALATKHGVGDVLVTEARARPGGNI
TTVERPDEGYLWEEGPNSFQPSDPVLTMAVDSGLKDDLVFGDPNAPRFVLWEGKLRPVPS
KPADLPFFDLMSIPGKLRAGFGALGIRPPPPGREESVEEFVRRNLGAEVFERLIEPFCSGVY
AGDPSKLSMKAAFGKVWRLEEAGGSIIGGTIKTIQERGKNPKPPRDPRLPTPKGQTVASFR
KGLAMLPNAITSSLGSKVKLSWKLLSITKSDGMGYVLVYETPEGVVSVQAKSVIMTIPSYVAS
DILRPLSSDAADALSRFYYPPMAAVTISYPKEAIRKECLIDGELQGFGQLHPRSQGVETLGTI
YSSSLFPNRAPAGRVLLLNYIGGATNTGIVSKSASELVEAVDRDLRKMLINPSAVDPLVLGV
RVWPQAIPQFLVGHLDLLEAAKSSLDRGGYDGLFLGGNYVAGVALGRCVEGAYESASQIS
DFLTKYAYK*
Setaria italica PPO1 T268L Y325F G367A (SEQ ID NO: 294)
AAVAGGAAEAPASAGARVSADCVVVGGGISGLCTAQALATKHGVGDVLVTEARARPGGNI
TTVERPDEGYLWEEGPNSFQPSDPVLTMAVDSGLKDDLVFGDPNAPRFVLWEGKLRPVPS
KPADLPFFDLMSIPGKLRAGFGALGIRPPPPGREESVEEFVRRNLGAEVFERLIEPFCSGVY
AGDPSKLSMKAAFGKVWRLEEAGGSIIGGTIKTIQERGKNPKPPRDPRLPTPKGQTVASFR
KGLAMLPNAITSSLGSKVKLSWKLLSITKSDGMGYVLVYETPEGVVSVQAKSVIMTIPSYVAS
DILRPLSSDAADALSRFYFPPVAAVTISYPKEAIRKECLIDGELQGFGQLHPRSQGVETLATIY
SSSLFPNRAPAGRVLLLNYIGGATNTGIVSKSASELVEAVDRDLRKMLINPSAVDPLVLGVR
VWPQAIPQFLVGHLDLLEAAKSSLDRGGYDGLFLGGNYVAGVALGRCVEGAYESASQISDF
LTKYAYK*
Setaria italica PPO1 T268L Y324T V238L T394R L442M (SEQ ID NO: 295)
AAVAGGAAEAPASAGARVSADCVVVGGGISGLCTAQALATKHGVGDVLVTEARARPGGNI
TTVERPDEGYLWEEGPNSFQPSDPVLTMAVDSGLKDDLVFGDPNAPRFVLWEGKLRPVPS
KPADLPFFDLMSIPGKLRAGFGALGIRPPPPGREESVEEFVRRNLGAEVFERLIEPFCSGVY
AGDPSKLSMKAAFGKVWRLEEAGGSIIGGTIKTIQERGKNPKPPRDPRLPTPKGQTVASFR
KGLAMLPNAITSSLGSKVKLSWKLLSITKSDGMGYVLVYETPEGVVSVQAKSVIMTIPSYVAS
DILRPLSSDAADALSRFTYPPLAAVTISYPKEAIRKECLIDGELQGFGQLHPRSQGVETLGTIY
SSSLFPNRAPAGRVLLLNYIGGAANTGIVSKSASELVEAVDRDLRKMLINPSAVDPLVLGVR
VWPQAIPQFMVGHLDLLEAAKSSLDRGGYDGLFLGGNYVAGVALGRCVEGAYESASQISD
FLTKYAYK*
Setaria italica PPO1 T268L Y389V L442M (SEQ ID NO: 296)
AAVAGGAAEAPASAGARVSADCVVVGGGISGLCTAQALATKHGVGDVLVTEARARPGGNI
TTVERPDEGYLWEEGPNSFQPSDPVLTMAVDSGLKDDLVFGDPNAPRFVLWEGKLRPVPS
KPADLPFFDLMSIPGKLRAGFGALGIRPPPPGREESVEEFVRRNLGAEVFERLIEPFCSGVY
AGDPSKLSMKAAFGKVWRLEEAGGSIIGGTIKTIQERGKNPKPPRDPRLPTPKGQTVASFR
KGLAMLPNAITSSLGSKVKLSWKLLSITKSDGMGYVLVYETPEGVVSVQAKSVIMTIPSYVAS
DILRPLSSDAADALSRFYYPPVAAVTISYPKEAIRKECLIDGELQGFGQLHPRSQGVETLGTI
YSSSLFPNRAPAGRVLLLNVIGGATNTGIVSKSASELVEAVDRDLRKMLINPSAVDPLVLGV
RVWPQAIPQFMVGHLDLLEAAKSSLDRGGYDGLFLGGNYVAGVALGRCVEGAYESASQIS
DFLTKYAYK*
Setaria italica PPO1 Y389M V424Q L442M (SEQ ID NO: 297)
AAVAGGAAEAPASAGARVSADCVVVGGGISGLCTAQALATKHGVGDVLVTEARARPGGNI
TTVERPDEGYLWEEGPNSFQPSDPVLTMAVDSGLKDDLVFGDPNAPRFVLWEGKLRPVPS
KPADLPFFDLMSIPGKLRAGFGALGIRPPPPGREESVEEFVRRNLGAEVFERLIEPFCSGVY
AGDPSKLSMKAAFGKVWRLEEAGGSIIGGTIKTIQERGKNPKPPRDPRLPTPKGQTVASFR
KGLAMLPNAITSSLGSKVKLSWKLTSITKSDGMGYVLVYETPEGVVSVQAKSVIMTIPSYVA
SDILRPLSSDAADALSRFYYPPVAAVTISYPKEAIRKECLIDGELQGFGQLHPRSQGVETLGT
IYSSSLFPNRAPAGRVLLLNMIGGATNTGIVSKSASELVEAVDRDLRKMLINPSAQDPLVLGV
RVWPQAIPQFMVGHLDLLEAAKSSLDRGGYDGLFLGGNYVAGVALGRCVEGAYESASQIS
DFLTKYAYK*
Setaria italica PPO1 T268L Y324D V328M (SEQ ID NO: 298)
AAVAGGAAEAPASAGARVSADCVVVGGGISGLCTAQALATKHGVGDVLVTEARARPGGNI
TTVERPDEGYLWEEGPNSFQPSDPVLTMAVDSGLKDDLVFGDPNAPRFVLWEGKLRPVPS
KPADLPFFDLMSIPGKLRAGFGALGIRPPPPGREESVEEFVRRNLGAEVFERLIEPFCSGVY
AGDPSKLSMKAAFGKVWRLEEAGGSIIGGTIKTIQERGKNPKPPRDPRLPTPKGQTVASFR
KGLAMLPNAITSSLGSKVKLSWKLLSITKSDGMGYVLVYETPEGVVSVQAKSVIMTIPSYVAS
DILRPLSSDAADALSRFDYPPMAAVTISYPKEAIRKECLIDGELQGFGQLHPRSQGVETLGTI
YSSSLFPNRAPAGRVLLLNYIGGATNTGIVSKSASELVEAVDRDLRKMLINPSAVDPLVLGV
RVWPQAIPQFLVGHLDLLEAAKSSLDRGGYDGLFLGGNYVAGVALGRCVEGAYESASQIS
DFLTKYAYK*
Setaria italica PPO1 T268L Y324C Y325F Y389M L442M (SEQ ID NO: 299)
AAVAGGAAEAPASAGARVSADCVVVGGGISGLCTAQALATKHGVGDVLVTEARARPGGNI
TTVERPDEGYLWEEGPNSFQPSDPVLTMAVDSGLKDDLVFGDPNAPRFVLWEGKLRPVPS
KPADLPFFDLMSIPGKLRAGFGALGIRPPPPGREESVEEFVRRNLGAEVFERLIEPFCSGVY
AGDPSKLSMKAAFGKVWRLEEAGGSIIGGTIKTIQERGKNPKPPRDPRLPTPKGQTVASFR
KGLAMLPNAITSSLGSKVKLSWKLLSITKSDGMGYVLVYETPEGVVSVQAKSVIMTIPSYVAS
DILRPLSSDAADALSRFCFPPVAAVTISYPKEAIRKECLIDGELQGFGQLHPRSQGVETLGTI
YSSSLFPNRAPAGRVLLLNMIGGATNTGIVSKSASELVEAVDRDLRKMLINPSAVDPLVLGV
RVWPQAIPQFMVGHLDLLEAAKSSLDRGGYDGLFLGGNYVAGVALGRCVEGAYESASQIS
DFLTKYAYK*
Setaria italica PPO1 Y324D V328L (SEQ ID NO: 300)
AAVAGGAAEAPASAGARVSADCVVVGGGISGLCTAQALATKHGVGDVLVTEARARPGGNI
TTVERPDEGYLWEEGPNSFQPSDPVLTMAVDSGLKDDLVFGDPNAPRFVLWEGKLRPVPS
KPADLPFFDLMSIPGKLRAGFGALGIRPPPPGREESVEEFVRRNLGAEVFERLIEPFCSGVY
AGDPSKLSMKAAFGKVWRLEEAGGSIIGGTIKTIQERGKNPKPPRDPRLPTPKGQTVASFR
KGLAMLPNAITSSLGSKVKLSWKLTSITKSDGMGYVLVYETPEGVVSVQAKSVIMTIPSYVA
SDILRPLSSDAADALSRFDYPPLAAVTISYPKEAIRKECLIDGELQGFGQLHPRSQGVETLGT
IYSSSLFPNRAPAGRVLLLNYIGGATNTGIVSKSASELVEAVDRDLRKMLINPSAVDPLVLGV
RVWPQAIPQFLVGHLDLLEAAKSSLDRGGYDGLFLGGNYVAGVALGRCVEGAYESASQIS
DFLTKYAYK*
Setaria italica PPO1 V328L G367A L442N (SEQ ID NO: 301)
AAVAGGAAEAPASAGARVSADCVVVGGGISGLCTAQALATKHGVGDVLVTEARARPGGNI
TTVERPDEGYLWEEGPNSFQPSDPVLTMAVDSGLKDDLVFGDPNAPRFVLWEGKLRPVPS
KPADLPFFDLMSIPGKLRAGFGALGIRPPPPGREESVEEFVRRNLGAEVFERLIEPFCSGVY
AGDPSKLSMKAAFGKVWRLEEAGGSIIGGTIKTIQERGKNPKPPRDPRLPTPKGQTVASFR
KGLAMLPNAITSSLGSKVKLSWKLTSITKSDGMGYVLVYETPEGVVSVQAKSVIMTIPSYVA
SDILRPLSSDAADALSRFYYPPLAAVTISYPKEAIRKECLIDGELQGFGQLHPRSQGVETLATI
YSSSLFPNRAPAGRVLLLNYIGGATNTGIVSKSASELVEAVDRDLRKMLINPSAVDPLVLGV
RVWPQAIPQFNVGHLDLLEAAKSSLDRGGYDGLFLGGNYVAGVALGRCVEGAYESASQIS
DFLTKYAYK*
Setaria italica PPO1 T268L G367A L442N (SEQ ID NO: 302)
AAVAGGAAEAPASAGARVSADCVVVGGGISGLCTAQALATKHGVGDVLVTEARARPGGNI
TTVERPDEGYLWEEGPNSFQPSDPVLTMAVDSGLKDDLVFGDPNAPRFVLWEGKLRPVPS
KPADLPFFDLMSIPGKLRAGFGALGIRPPPPGREESVEEFVRRNLGAEVFERLIEPFCSGVY
AGDPSKLSMKAAFGKVWRLEEAGGSIIGGTIKTIQERGKNPKPPRDPRLPTPKGQTVASFR
KGLAMLPNAITSSLGSKVKLSWKLLSITKSDGMGYVLVYETPEGVVSVQAKSVIMTIPSYVAS
DILRPLSSDAADALSRFYYPPVAAVTISYPKEAIRKECLIDGELQGFGQLHPRSQGVETLATIY
SSSLFPNRAPAGRVLLLNYIGGATNTGIVSKSASELVEAVDRDLRKMLINPSAVDPLVLGVR
VWPQAIPQFNVGHLDLLEAAKSSLDRGGYDGLFLGGNYVAGVALGRCVEGAYESASQISD
FLTKYAYK*
(Transit peptides)
Arabidopsis thaliana PPO1 WT chloroplastic transit peptide (SEQ ID NO: 303)
MELSLLRPTTQSLLPSFSKPNLRLNVYKPLRLRC
Setaria italica PPO1 WT chloroplastic transit peptide (SEQ ID NO: 304)
MVAAAMATAPSAGVPPLRGTRGPARFRIRGVSVRC
Arabidopsis thaliana PPO1 K270M (with transit peptide) (SEQ ID NO: 305)
MELSLLRPTTQSLLPSFSKPNLRLNVYKPLRLRCSVAGGPTVGSSKIEGGGGTTITTDCVIV
GGGISGLCIAQALATKHPDAAPNLIVTEAKDRVGGNIITREENGFLWEEGPNSFQPSDPMLT
MVVDSGLKDDLVLGDPTAPRFVLWNGKLRPVPSKLTDLPFFDLMSIGGKIRAGFGALGIRPS
PPGREESVEEFVRRNLGDEVFERLIEPFCSGVYAGDPSKLSMKAAFGKVWKLEQNGGSIIG
GTFKAIQERKNAPKAERDPRLPMPQGQTVGSFRKGLRMLPEAISARLGSKVKLSWKLSGIT
KLESGGYNLTYETPDGLVSVQSKSVVMTVPSHVASGLLRPLSESAANALSKLYYPPVAAVSI
SYPKEAIRTECLIDGELKGFGQLHPRTQGVETLGTIYSSSLFPNRAPPGRILLLNYIGGSTNT
GILSKSEGELVEAVDRDLRKMLIKPNSTDPLKLGVRVWPQAIPQFLVGHFDILDTAKSSLTSS
GYEGLFLGGNYVAGVALGRCVEGAYETAIEVNNFMSRYAYK*
Arabidopsis thaliana PPO1 Y361C (with transit peptide) (SEQ ID NO: 306)
MELSLLRPTTQSLLPSFSKPNLRLNVYKPLRLRCSVAGGPTVGSSKIEGGGGTTITTDCVIV
GGGISGLCIAQALATKHPDAAPNLIVTEAKDRVGGNIITREENGFLWEEGPNSFQPSDPMLT
MVVDSGLKDDLVLGDPTAPRFVLWNGKLRPVPSKLTDLPFFDLMSIGGKIRAGFGALGIRPS
PPGREESVEEFVRRNLGDEVFERLIEPFCSGVYAGDPSKLSMKAAFGKVWKLEQNGGSIIG
GTFKAIQERKNAPKAERDPRLPKPQGQTVGSFRKGLRMLPEAISARLGSKVKLSWKLSGIT
KLESGGYNLTYETPDGLVSVQSKSVVMTVPSHVASGLLRPLSESAANALSKLCYPPVAAVSI
SYPKEAIRTECLIDGELKGFGQLHPRTQGVETLGTIYSSSLFPNRAPPGRILLLNYIGGSTNT
GILSKSEGELVEAVDRDLRKMLIKPNSTDPLKLGVRVWPQAIPQFLVGHFDILDTAKSSLTSS
GYEGLFLGGNYVAGVALGRCVEGAYETAIEVNNFMSRYAYK*
Arabidopsis thaliana PPO1 Y362C (with transit peptide) (SEQ ID NO: 307)
MELSLLRPTTQSLLPSFSKPNLRLNVYKPLRLRCSVAGGPTVGSSKIEGGGGTTITTDCVIV
GGGISGLCIAQALATKHPDAAPNLIVTEAKDRVGGNIITREENGFLWEEGPNSFQPSDPMLT
MVVDSGLKDDLVLGDPTAPRFVLWNGKLRPVPSKLTDLPFFDLMSIGGKIRAGFGALGIRPS
PPGREESVEEFVRRNLGDEVFERLIEPFCSGVYAGDPSKLSMKAAFGKVWKLEQNGGSIIG
GTFKAIQERKNAPKAERDPRLPKPQGQTVGSFRKGLRMLPEAISARLGSKVKLSWKLSGIT
KLESGGYNLTYETPDGLVSVQSKSVVMTVPSHVASGLLRPLSESAANALSKLYCPPVAAVSI
SYPKEAIRTECLIDGELKGFGQLHPRTQGVETLGTIYSSSLFPNRAPPGRILLLNYIGGSTNT
GILSKSEGELVEAVDRDLRKMLIKPNSTDPLKLGVRVWPQAIPQFLVGHFDILDTAKSSLTSS
GYEGLFLGGNYVAGVALGRCVEGAYETAIEVNNFMSRYAYK*
Arabidopsis thaliana PPO1 T431F (with transit peptide) (SEQ ID NO: 308)
MELSLLRPTTQSLLPSFSKPNLRLNVYKPLRLRCSVAGGPTVGSSKIEGGGGTTITTDCVIV
GGGISGLCIAQALATKHPDAAPNLIVTEAKDRVGGNIITREENGFLWEEGPNSFQPSDPMLT
MVVDSGLKDDLVLGDPTAPRFVLWNGKLRPVPSKLTDLPFFDLMSIGGKIRAGFGALGIRPS
PPGREESVEEFVRRNLGDEVFERLIEPFCSGVYAGDPSKLSMKAAFGKVWKLEQNGGSIIG
GTFKAIQERKNAPKAERDPRLPKPQGQTVGSFRKGLRMLPEAISARLGSKVKLSWKLSGIT
KLESGGYNLTYETPDGLVSVQSKSVVMTVPSHVASGLLRPLSESAANALSKLYYPPVAAVSI
SYPKEAIRTECLIDGELKGFGQLHPRTQGVETLGTIYSSSLFPNRAPPGRILLLNYIGGSENT
GILSKSEGELVEAVDRDLRKMLIKPNSTDPLKLGVRVWPQAIPQFLVGHFDILDTAKSSLTSS
GYEGLFLGGNYVAGVALGRCVEGAYETAIEVNNFMSRYAYK*
Arabidopsis thaliana PPO1 L464Y (with transit peptide) (SEQ ID NO: 309)
MELSLLRPTTQSLLPSFSKPNLRLNVYKPLRLRCSVAGGPTVGSSKIEGGGGTTITTDCVIV
GGGISGLCIAQALATKHPDAAPNLIVTEAKDRVGGNIITREENGFLWEEGPNSFQPSDPMLT
MVVDSGLKDDLVLGDPTAPRFVLWNGKLRPVPSKLTDLPFFDLMSIGGKIRAGFGALGIRPS
PPGREESVEEFVRRNLGDEVFERLIEPFCSGVYAGDPSKLSMKAAFGKVWKLEQNGGSIIG
GTFKAIQERKNAPKAERDPRLPKPQGQTVGSFRKGLRMLPEAISARLGSKVKLSWKLSGIT
KLESGGYNLTYETPDGLVSVQSKSVVMTVPSHVASGLLRPLSESAANALSKLYYPPVAAVSI
SYPKEAIRTECLIDGELKGFGQLHPRTQGVETLGTIYSSSLFPNRAPPGRILLLNYIGGSTNT
GILSKSEGELVEAVDRDLRKMLIKPNSTDPYKLGVRVWPQAIPQFLVGHFDILDTAKSSLTSS
GYEGLFLGGNYVAGVALGRCVEGAYETAIEVNNFMSRYAYK*
Arabidopsis thaliana PPO1 G481D (with transit peptide) (SEQ ID NO: 310)
MELSLLRPTTQSLLPSFSKPNLRLNVYKPLRLRCSVAGGPTVGSSKIEGGGGTTITTDCVIV
GGGISGLCIAQALATKHPDAAPNLIVTEAKDRVGGNIITREENGFLWEEGPNSFQPSDPMLT
MVVDSGLKDDLVLGDPTAPRFVLWNGKLRPVPSKLTDLPFFDLMSIGGKIRAGFGALGIRPS
PPGREESVEEFVRRNLGDEVFERLIEPFCSGVYAGDPSKLSMKAAFGKVWKLEQNGGSIIG
GTFKAIQERKNAPKAERDPRLPKPQGQTVGSFRKGLRMLPEAISARLGSKVKLSWKLSGIT
KLESGGYNLTYETPDGLVSVQSKSVVMTVPSHVASGLLRPLSESAANALSKLYYPPVAAVSI
SYPKEAIRTECLIDGELKGFGQLHPRTQGVETLGTIYSSSLFPNRAPPGRILLLNYIGGSTNT
GILSKSEGELVEAVDRDLRKMLIKPNSTDPLKLGVRVWPQAIPQFLVDHFDILDTAKSSLTSS
GYEGLFLGGNYVAGVALGRCVEGAYETAIEVNNFMSRYAYK*
Arabidopsis thaliana PPO1 G481K (with transit peptide) (SEQ ID NO: 311)
MELSLLRPTTQSLLPSFSKPNLRLNVYKPLRLRCSVAGGPTVGSSKIEGGGGTTITTDCVIV
GGGISGLCIAQALATKHPDAAPNLIVTEAKDRVGGNIITREENGFLWEEGPNSFQPSDPMLT
MVVDSGLKDDLVLGDPTAPRFVLWNGKLRPVPSKLTDLPFFDLMSIGGKIRAGFGALGIRPS
PPGREESVEEFVRRNLGDEVFERLIEPFCSGVYAGDPSKLSMKAAFGKVWKLEQNGGSIIG
GTFKAIQERKNAPKAERDPRLPKPQGQTVGSFRKGLRMLPEAISARLGSKVKLSWKLSGIT
KLESGGYNLTYETPDGLVSVQSKSVVMTVPSHVASGLLRPLSESAANALSKLYYPPVAAVSI
SYPKEAIRTECLIDGELKGFGQLHPRTQGVETLGTIYSSSLFPNRAPPGRILLLNYIGGSTNT
GILSKSEGELVEAVDRDLRKMLIKPNSTDPLKLGVRVWPQAIPQFLVKHFDILDTAKSSLTSS
GYEGLFLGGNYVAGVALGRCVEGAYETAIEVNNFMSRYAYK*
Arabidopsis thaliana PPO1 G481N (with transit peptide) (SEQ ID NO: 312)
MELSLLRPTTQSLLPSFSKPNLRLNVYKPLRLRCSVAGGPTVGSSKIEGGGGTTITTDCVIV
GGGISGLCIAQALATKHPDAAPNLIVTEAKDRVGGNIITREENGFLWEEGPNSFQPSDPMLT
MVVDSGLKDDLVLGDPTAPRFVLWNGKLRPVPSKLTDLPFFDLMSIGGKIRAGFGALGIRPS
PPGREESVEEFVRRNLGDEVFERLIEPFCSGVYAGDPSKLSMKAAFGKVWKLEQNGGSIIG
GTFKAIQERKNAPKAERDPRLPKPQGQTVGSFRKGLRMLPEAISARLGSKVKLSWKLSGIT
KLESGGYNLTYETPDGLVSVQSKSVVMTVPSHVASGLLRPLSESAANALSKLYYPPVAAVSI
SYPKEAIRTECLIDGELKGFGQLHPRTQGVETLGTIYSSSLFPNRAPPGRILLLNYIGGSTNT
GILSKSEGELVEAVDRDLRKMLIKPNSTDPLKLGVRVWPQAIPQFLVNHFDILDTAKSSLTSS
GYEGLFLGGNYVAGVALGRCVEGAYETAIEVNNFMSRYAYK*
Setaria italica PPO1 T268M (with transit peptide) (SEQ ID NO: 313)
MVAAAMATAPSAGVPPLRGTRGPARFRIRGVSVRCAAVAGGAAEAPASAGARVSADCVVV
GGGISGLCTAQALATKHGVGDVLVTEARARPGGNITTVERPDEGYLWEEGPNSFQPSDPV
LTMAVDSGLKDDLVFGDPNAPRFVLWEGKLRPVPSKPADLPFFDLMSIPGKLRAGFGALGI
RPPPPGREESVEEFVRRNLGAEVFERLIEPFCSGVYAGDPSKLSMKAAFGKVWRLEEAGG
SIIGGTIKTIQERGKNPKPPRDPRLPMPKGQTVASFRKGLAMLPNAITSSLGSKVKLSWKLTS
ITKSDGMGYVLVYETPEGVVSVQAKSVIMTIPSYVASDILRPLSSDAADALSRFYYPPVAAVT
ISYPKEAIRKECLIDGELQGFGQLHPRSQGVETLGTIYSSSLFPNRAPAGRVLLLNYIGGATN
TGIVSKSASELVEAVDRDLRKMLINPSAVDPLVLGVRVWPQAIPQFLVGHLDLLEAAKSSLD
RGGYDGLFLGGNYVAGVALGRCVEGAYESASQISDFLTKYAYK*
Setaria italica PPO1 Y359C (with transit peptide) (SEQ ID NO: 314)
MVAAAMATAPSAGVPPLRGTRGPARFRIRGVSVRCAAVAGGAAEAPASAGARVSADCVVV
GGGISGLCTAQALATKHGVGDVLVTEARARPGGNITTVERPDEGYLWEEGPNSFQPSDPV
LTMAVDSGLKDDLVFGDPNAPRFVLWEGKLRPVPSKPADLPFFDLMSIPGKLRAGFGALGI
RPPPPGREESVEEFVRRNLGAEVFERLIEPFCSGVYAGDPSKLSMKAAFGKVWRLEEAGG
SIIGGTIKTIQERGKNPKPPRDPRLPTPKGQTVASFRKGLAMLPNAITSSLGSKVKLSWKLTSI
TKSDGMGYVLVYETPEGVVSVQAKSVIMTIPSYVASDILRPLSSDAADALSRFCYPPVAAVTI
SYPKEAIRKECLIDGELQGFGQLHPRSQGVETLGTIYSSSLFPNRAPAGRVLLLNYIGGATNT
GIVSKSASELVEAVDRDLRKMLINPSAVDPLVLGVRVWPQAIPQFLVGHLDLLEAAKSSLDR
GGYDGLFLGGNYVAGVALGRCVEGAYESASQISDFLTKYAYK*
Setaria italica PPO1 Y360C (with transit peptide) (SEQ ID NO: 315)
MVAAAMATAPSAGVPPLRGTRGPARFRIRGVSVRCAAVAGGAAEAPASAGARVSADCVVV
GGGISGLCTAQALATKHGVGDVLVTEARARPGGNITTVERPDEGYLWEEGPNSFQPSDPV
LTMAVDSGLKDDLVFGDPNAPRFVLWEGKLRPVPSKPADLPFFDLMSIPGKLRAGFGALGI
RPPPPGREESVEEFVRRNLGAEVFERLIEPFCSGVYAGDPSKLSMKAAFGKVWRLEEAGG
SIIGGTIKTIQERGKNPKPPRDPRLPTPKGQTVASFRKGLAMLPNAITSSLGSKVKLSWKLTSI
TKSDGMGYVLVYETPEGVVSVQAKSVIMTIPSYVASDILRPLSSDAADALSRFYCPPVAAVTI
SYPKEAIRKECLIDGELQGFGQLHPRSQGVETLGTIYSSSLFPNRAPAGRVLLLNYIGGATNT
GIVSKSASELVEAVDRDLRKMLINPSAVDPLVLGVRVWPQAIPQFLVGHLDLLEAAKSSLDR
GGYDGLFLGGNYVAGVALGRCVEGAYESASQISDFLTKYAYK*
Setaria italica PPO1 T429F (with transit peptide) (SEQ ID NO: 316)
MVAAAMATAPSAGVPPLRGTRGPARFRIRGVSVRCAAVAGGAAEAPASAGARVSADCVVV
GGGISGLCTAQALATKHGVGDVLVTEARARPGGNITTVERPDEGYLWEEGPNSFQPSDPV
LTMAVDSGLKDDLVFGDPNAPRFVLWEGKLRPVPSKPADLPFFDLMSIPGKLRAGFGALGI
RPPPPGREESVEEFVRRNLGAEVFERLIEPFCSGVYAGDPSKLSMKAAFGKVWRLEEAGG
SIIGGTIKTIQERGKNPKPPRDPRLPTPKGQTVASFRKGLAMLPNAITSSLGSKVKLSWKLTSI
TKSDGMGYVLVYETPEGVVSVQAKSVIMTIPSYVASDILRPLSSDAADALSRFYYPPVAAVTI
SYPKEAIRKECLIDGELQGFGQLHPRSQGVETLGTIYSSSLFPNRAPAGRVLLLNYIGGAFNT
GIVSKSASELVEAVDRDLRKMLINPSAVDPLVLGVRVWPQAIPQFLVGHLDLLEAAKSSLDR
GGYDGLFLGGNYVAGVALGRCVEGAYESASQISDFLTKYAYK*
Setaria italica PPO1 L462Y (with transit peptide) (SEQ ID NO: 317)
MVAAAMATAPSAGVPPLRGTRGPARFRIRGVSVRCAAVAGGAAEAPASAGARVSADCVVV
GGGISGLCTAQALATKHGVGDVLVTEARARPGGNITTVERPDEGYLWEEGPNSFQPSDPV
LTMAVDSGLKDDLVFGDPNAPRFVLWEGKLRPVPSKPADLPFFDLMSIPGKLRAGFGALGI
RPPPPGREESVEEFVRRNLGAEVFERLIEPFCSGVYAGDPSKLSMKAAFGKVWRLEEAGG
SIIGGTIKTIQERGKNPKPPRDPRLPTPKGQTVASFRKGLAMLPNAITSSLGSKVKLSWKLTSI
TKSDGMGYVLVYETPEGVVSVQAKSVIMTIPSYVASDILRPLSSDAADALSRFYYPPVAAVTI
SYPKEAIRKECLIDGELQGFGQLHPRSQGVETLGTIYSSSLFPNRAPAGRVLLLNYIGGATNT
GIVSKSASELVEAVDRDLRKMLINPSAVDPYVLGVRVWPQAIPQFLVGHLDLLEAAKSSLDR
GGYDGLFLGGNYVAGVALGRCVEGAYESASQISDFLTKYAYK*
Setaria italica PPO1 G479D (with transit peptide) (SEQ ID NO: 318)
MVAAAMATAPSAGVPPLRGTRGPARFRIRGVSVRCAAVAGGAAEAPASAGARVSADCVVV
GGGISGLCTAQALATKHGVGDVLVTEARARPGGNITTVERPDEGYLWEEGPNSFQPSDPV
LTMAVDSGLKDDLVFGDPNAPRFVLWEGKLRPVPSKPADLPFFDLMSIPGKLRAGFGALGI
RPPPPGREESVEEFVRRNLGAEVFERLIEPFCSGVYAGDPSKLSMKAAFGKVWRLEEAGG
SIIGGTIKTIQERGKNPKPPRDPRLPTPKGQTVASFRKGLAMLPNAITSSLGSKVKLSWKLTSI
TKSDGMGYVLVYETPEGVVSVQAKSVIMTIPSYVASDILRPLSSDAADALSRFYYPPVAAVTI
SYPKEAIRKECLIDGELQGFGQLHPRSQGVETLGTIYSSSLFPNRAPAGRVLLLNYIGGATNT
GIVSKSASELVEAVDRDLRKMLINPSAVDPLVLGVRVWPQAIPQFLVDHLDLLEAAKSSLDR
GGYDGLFLGGNYVAGVALGRCVEGAYESASQISDFLTKYAYK*
Setaria italica PPO1 G479K (with transit peptide) (SEQ ID NO: 319)
MVAAAMATAPSAGVPPLRGTRGPARFRIRGVSVRCAAVAGGAAEAPASAGARVSADCVVV
GGGISGLCTAQALATKHGVGDVLVTEARARPGGNITTVERPDEGYLWEEGPNSFQPSDPV
LTMAVDSGLKDDLVFGDPNAPRFVLWEGKLRPVPSKPADLPFFDLMSIPGKLRAGFGALGI
RPPPPGREESVEEFVRRNLGAEVFERLIEPFCSGVYAGDPSKLSMKAAFGKVWRLEEAGG
SIIGGTIKTIQERGKNPKPPRDPRLPTPKGQTVASFRKGLAMLPNAITSSLGSKVKLSWKLTSI
TKSDGMGYVLVYETPEGVVSVQAKSVIMTIPSYVASDILRPLSSDAADALSRFYYPPVAAVTI
SYPKEAIRKECLIDGELQGFGQLHPRSQGVETLGTIYSSSLFPNRAPAGRVLLLNYIGGATNT
GIVSKSASELVEAVDRDLRKMLINPSAVDPLVLGVRVWPQAIPQFLVKHLDLLEAAKSSLDR
GGYDGLFLGGNYVAGVALGRCVEGAYESASQISDFLTKYAYK*
Setaria italica PPO1 G479N (with transit peptide) (SEQ ID NO: 320)
MVAAAMATAPSAGVPPLRGTRGPARFRIRGVSVRCAAVAGGAAEAPASAGARVSADCVVV
GGGISGLCTAQALATKHGVGDVLVTEARARPGGNITTVERPDEGYLWEEGPNSFQPSDPV
LTMAVDSGLKDDLVFGDPNAPRFVLWEGKLRPVPSKPADLPFFDLMSIPGKLRAGFGALGI
RPPPPGREESVEEFVRRNLGAEVFERLIEPFCSGVYAGDPSKLSMKAAFGKVWRLEEAGG
SIIGGTIKTIQERGKNPKPPRDPRLPTPKGQTVASFRKGLAMLPNAITSSLGSKVKLSWKLTSI
TKSDGMGYVLVYETPEGVVSVQAKSVIMTIPSYVASDILRPLSSDAADALSRFYYPPVAAVTI
SYPKEAIRKECLIDGELQGFGQLHPRSQGVETLGTIYSSSLFPNRAPAGRVLLLNYIGGATNT
GIVSKSASELVEAVDRDLRKMLINPSAVDPLVLGVRVWPQAIPQFLVNHLDLLEAAKSSLDR
GGYDGLFLGGNYVAGVALGRCVEGAYESASQISDFLTKYAYK*
Arabidopsis thaliana PPO1 K236M (without transit peptide) (SEQ ID NO: 321)
SVAGGPTVGSSKIEGGGGTTITTDCVIVGGGISGLCIAQALATKHPDAAPNLIVTEAKDRVGG
NIITREENGFLWEEGPNSFQPSDPMLTMVVDSGLKDDLVLGDPTAPRFVLWNGKLRPVPSK
LTDLPFFDLMSIGGKIRAGFGALGIRPSPPGREESVEEFVRRNLGDEVFERLIEPFCSGVYA
GDPSKLSMKAAFGKVWKLEQNGGSIIGGTFKAIQERKNAPKAERDPRLPMPQGQTVGSFR
KGLRMLPEAISARLGSKVKLSWKLSGITKLESGGYNLTYETPDGLVSVQSKSVVMTVPSHV
ASGLLRPLSESAANALSKLYYPPVAAVSISYPKEAIRTECLIDGELKGFGQLHPRTQGVETLG
TIYSSSLFPNRAPPGRILLLNYIGGSTNTGILSKSEGELVEAVDRDLRKMLIKPNSTDPLKLGV
RVWPQAIPQFLVGHFDILDTAKSSLTSSGYEGLFLGGNYVAGVALGRCVEGAYETAIEVNNF
MSRYAYK*
Arabidopsis thaliana PPO1 Y327C (without transit peptide) (SEQ ID NO: 322)
SVAGGPTVGSSKIEGGGGTTITTDCVIVGGGISGLCIAQALATKHPDAAPNLIVTEAKDRVGG
NIITREENGFLWEEGPNSFQPSDPMLTMVVDSGLKDDLVLGDPTAPRFVLWNGKLRPVPSK
LTDLPFFDLMSIGGKIRAGFGALGIRPSPPGREESVEEFVRRNLGDEVFERLIEPFCSGVYA
GDPSKLSMKAAFGKVWKLEQNGGSIIGGTFKAIQERKNAPKAERDPRLPKPQGQTVGSFR
KGLRMLPEAISARLGSKVKLSWKLSGITKLESGGYNLTYETPDGLVSVQSKSVVMTVPSHV
ASGLLRPLSESAANALSKLCYPPVAAVSISYPKEAIRTECLIDGELKGFGQLHPRTQGVETLG
TIYSSSLFPNRAPPGRILLLNYIGGSTNTGILSKSEGELVEAVDRDLRKMLIKPNSTDPLKLGV
RVWPQAIPQFLVGHFDILDTAKSSLTSSGYEGLFLGGNYVAGVALGRCVEGAYETAIEVNNF
MSRYAYK*
Arabidopsis thaliana PPO1 Y328C (without transit peptide) (SEQ ID NO: 323)
SVAGGPTVGSSKIEGGGGTTITTDCVIVGGGISGLCIAQALATKHPDAAPNLIVTEAKDRVGG
NIITREENGFLWEEGPNSFQPSDPMLTMVVDSGLKDDLVLGDPTAPRFVLWNGKLRPVPSK
LTDLPFFDLMSIGGKIRAGFGALGIRPSPPGREESVEEFVRRNLGDEVFERLIEPFCSGVYA
GDPSKLSMKAAFGKVWKLEQNGGSIIGGTFKAIQERKNAPKAERDPRLPKPQGQTVGSFR
KGLRMLPEAISARLGSKVKLSWKLSGITKLESGGYNLTYETPDGLVSVQSKSVVMTVPSHV
ASGLLRPLSESAANALSKLYCPPVAAVSISYPKEAIRTECLIDGELKGFGQLHPRTQGVETLG
TIYSSSLFPNRAPPGRILLLNYIGGSTNTGILSKSEGELVEAVDRDLRKMLIKPNSTDPLKLGV
RVWPQAIPQFLVGHFDILDTAKSSLTSSGYEGLFLGGNYVAGVALGRCVEGAYETAIEVNNF
MSRYAYK*
Arabidopsis thaliana PPO1 T397F (without transit peptide) (SEQ ID NO: 324)
SVAGGPTVGSSKIEGGGGTTITTDCVIVGGGISGLCIAQALATKHPDAAPNLIVTEAKDRVGG
NIITREENGFLWEEGPNSFQPSDPMLTMVVDSGLKDDLVLGDPTAPRFVLWNGKLRPVPSK
LTDLPFFDLMSIGGKIRAGFGALGIRPSPPGREESVEEFVRRNLGDEVFERLIEPFCSGVYA
GDPSKLSMKAAFGKVWKLEQNGGSIIGGTFKAIQERKNAPKAERDPRLPKPQGQTVGSFR
KGLRMLPEAISARLGSKVKLSWKLSGITKLESGGYNLTYETPDGLVSVQSKSVVMTVPSHV
ASGLLRPLSESAANALSKLYYPPVAAVSISYPKEAIRTECLIDGELKGFGQLHPRTQGVETLG
TIYSSSLFPNRAPPGRILLLNYIGGSFNTGILSKSEGELVEAVDRDLRKMLIKPNSTDPLKLGV
RVWPQAIPQFLVGHFDILDTAKSSLTSSGYEGLFLGGNYVAGVALGRCVEGAYETAIEVNNF
MSRYAYK*
Arabidopsis thaliana PPO1 L430Y (without transit peptide) (SEQ ID NO: 325)
SVAGGPTVGSSKIEGGGGTTITTDCVIVGGGISGLCIAQALATKHPDAAPNLIVTEAKDRVGG
NIITREENGFLWEEGPNSFQPSDPMLTMVVDSGLKDDLVLGDPTAPRFVLWNGKLRPVPSK
LTDLPFFDLMSIGGKIRAGFGALGIRPSPPGREESVEEFVRRNLGDEVFERLIEPFCSGVYA
GDPSKLSMKAAFGKVWKLEQNGGSIIGGTFKAIQERKNAPKAERDPRLPKPQGQTVGSFR
KGLRMLPEAISARLGSKVKLSWKLSGITKLESGGYNLTYETPDGLVSVQSKSVVMTVPSHV
ASGLLRPLSESAANALSKLYYPPVAAVSISYPKEAIRTECLIDGELKGFGQLHPRTQGVETLG
TIYSSSLFPNRAPPGRILLLNYIGGSTNTGILSKSEGELVEAVDRDLRKMLIKPNSTDPYKLGV
RVWPQAIPQFLVGHFDILDTAKSSLTSSGYEGLFLGGNYVAGVALGRCVEGAYETAIEVNNF
MSRYAYK*
Arabidopsis thaliana PPO1 G447D (without transit peptide) (SEQ ID NO: 326)
SVAGGPTVGSSKIEGGGGTTITTDCVIVGGGISGLCIAQALATKHPDAAPNLIVTEAKDRVGG
NIITREENGFLWEEGPNSFQPSDPMLTMVVDSGLKDDLVLGDPTAPRFVLWNGKLRPVPSK
LTDLPFFDLMSIGGKIRAGFGALGIRPSPPGREESVEEFVRRNLGDEVFERLIEPFCSGVYA
GDPSKLSMKAAFGKVWKLEQNGGSIIGGTFKAIQERKNAPKAERDPRLPKPQGQTVGSFR
KGLRMLPEAISARLGSKVKLSWKLSGITKLESGGYNLTYETPDGLVSVQSKSVVMTVPSHV
ASGLLRPLSESAANALSKLYYPPVAAVSISYPKEAIRTECLIDGELKGFGQLHPRTQGVETLG
TIYSSSLFPNRAPPGRILLLNYIGGSTNTGILSKSEGELVEAVDRDLRKMLIKPNSTDPLKLGV
RVWPQAIPQFLVDHFDILDTAKSSLTSSGYEGLFLGGNYVAGVALGRCVEGAYETAIEVNNF
MSRYAYK*
Arabidopsis thaliana PPO1 G447K (without transit peptide) (SEQ ID NO: 327)
SVAGGPTVGSSKIEGGGGTTITTDCVIVGGGISGLCIAQALATKHPDAAPNLIVTEAKDRVGG
NIITREENGFLWEEGPNSFQPSDPMLTMVVDSGLKDDLVLGDPTAPRFVLWNGKLRPVPSK
LTDLPFFDLMSIGGKIRAGFGALGIRPSPPGREESVEEFVRRNLGDEVFERLIEPFCSGVYA
GDPSKLSMKAAFGKVWKLEQNGGSIIGGTFKAIQERKNAPKAERDPRLPKPQGQTVGSFR
KGLRMLPEAISARLGSKVKLSWKLSGITKLESGGYNLTYETPDGLVSVQSKSVVMTVPSHV
ASGLLRPLSESAANALSKLYYPPVAAVSISYPKEAIRTECLIDGELKGFGQLHPRTQGVETLG
TIYSSSLFPNRAPPGRILLLNYIGGSTNTGILSKSEGELVEAVDRDLRKMLIKPNSTDPLKLGV
RVWPQAIPQFLVKHFDILDTAKSSLTSSGYEGLFLGGNYVAGVALGRCVEGAYETAIEVNNF
MSRYAYK*
Arabidopsis thaliana PPO1 G447N (without transit peptide) (SEQ ID NO: 328)
SVAGGPTVGSSKIEGGGGTTITTDCVIVGGGISGLCIAQALATKHPDAAPNLIVTEAKDRVGG
NIITREENGFLWEEGPNSFQPSDPMLTMVVDSGLKDDLVLGDPTAPRFVLWNGKLRPVPSK
LTDLPFFDLMSIGGKIRAGFGALGIRPSPPGREESVEEFVRRNLGDEVFERLIEPFCSGVYA
GDPSKLSMKAAFGKVWKLEQNGGSIIGGTFKAIQERKNAPKAERDPRLPKPQGQTVGSFR
KGLRMLPEAISARLGSKVKLSWKLSGITKLESGGYNLTYETPDGLVSVQSKSVVMTVPSHV
ASGLLRPLSESAANALSKLYYPPVAAVSISYPKEAIRTECLIDGELKGFGQLHPRTQGVETLG
TIYSSSLFPNRAPPGRILLLNYIGGSTNTGILSKSEGELVEAVDRDLRKMLIKPNSTDPLKLGV
RVWPQAIPQFLVNHFDILDTAKSSLTSSGYEGLFLGGNYVAGVALGRCVEGAYETAIEVNNF
MSRYAYK*
Setaria italica PPO1 T233M (without transit peptide) (SEQ ID NO: 329)
AAVAGGAAEAPASAGARVSADCVVVGGGISGLCTAQALATKHGVGDVLVTEARARPGGNI
TTVERPDEGYLWEEGPNSFQPSDPVLTMAVDSGLKDDLVFGDPNAPRFVLWEGKLRPVPS
KPADLPFFDLMSIPGKLRAGFGALGIRPPPPGREESVEEFVRRNLGAEVFERLIEPFCSGVY
AGDPSKLSMKAAFGKVWRLEEAGGSIIGGTIKTIQERGKNPKPPRDPRLPMPKGQTVASFR
KGLAMLPNAITSSLGSKVKLSWKLTSITKSDGMGYVLVYETPEGVVSVQAKSVIMTIPSYVA
SDILRPLSSDAADALSRFYYPPVAAVTISYPKEAIRKECLIDGELQGFGQLHPRSQGVETLGT
IYSSSLFPNRAPAGRVLLLNYIGGATNTGIVSKSASELVEAVDRDLRKMLINPSAVDPLVLGV
RVWPQAIPQFLVGHLDLLEAAKSSLDRGGYDGLFLGGNYVAGVALGRCVEGAYESASQIS
DFLTKYAYK*
Setaria italica PPO1 Y324C (without transit peptide) (SEQ ID NO: 330)
AAVAGGAAEAPASAGARVSADCVVVGGGISGLCTAQALATKHGVGDVLVTEARARPGGNI
TTVERPDEGYLWEEGPNSFQPSDPVLTMAVDSGLKDDLVFGDPNAPRFVLWEGKLRPVPS
KPADLPFFDLMSIPGKLRAGFGALGIRPPPPGREESVEEFVRRNLGAEVFERLIEPFCSGVY
AGDPSKLSMKAAFGKVWRLEEAGGSIIGGTIKTIQERGKNPKPPRDPRLPTPKGQTVASFR
KGLAMLPNAITSSLGSKVKLSWKLTSITKSDGMGYVLVYETPEGVVSVQAKSVIMTIPSYVA
SDILRPLSSDAADALSRFCYPPVAAVTISYPKEAIRKECLIDGELQGFGQLHPRSQGVETLGT
IYSSSLFPNRAPAGRVLLLNYIGGATNTGIVSKSASELVEAVDRDLRKMLINPSAVDPLVLGV
RVWPQAIPQFLVGHLDLLEAAKSSLDRGGYDGLFLGGNYVAGVALGRCVEGAYESASQIS
DFLTKYAYK*
Setaria italica PPO1 Y325C (without transit peptide) (SEQ ID NO: 331)
AAVAGGAAEAPASAGARVSADCVVVGGGISGLCTAQALATKHGVGDVLVTEARARPGGNI
TTVERPDEGYLWEEGPNSFQPSDPVLTMAVDSGLKDDLVFGDPNAPRFVLWEGKLRPVPS
KPADLPFFDLMSIPGKLRAGFGALGIRPPPPGREESVEEFVRRNLGAEVFERLIEPFCSGVY
AGDPSKLSMKAAFGKVWRLEEAGGSIIGGTIKTIQERGKNPKPPRDPRLPTPKGQTVASFR
KGLAMLPNAITSSLGSKVKLSWKLTSITKSDGMGYVLVYETPEGVVSVQAKSVIMTIPSYVA
SDILRPLSSDAADALSRFYCPPVAAVTISYPKEAIRKECLIDGELQGFGQLHPRSQGVETLGT
IYSSSLFPNRAPAGRVLLLNYIGGATNTGIVSKSASELVEAVDRDLRKMLINPSAVDPLVLGV
RVWPQAIPQFLVGHLDLLEAAKSSLDRGGYDGLFLGGNYVAGVALGRCVEGAYESASQIS
DFLTKYAYK*
Setaria italica PPO1 T394F (without transit peptide) (SEQ ID NO: 332)
AAVAGGAAEAPASAGARVSADCVVVGGGISGLCTAQALATKHGVGDVLVTEARARPGGNI
TTVERPDEGYLWEEGPNSFQPSDPVLTMAVDSGLKDDLVFGDPNAPRFVLWEGKLRPVPS
KPADLPFFDLMSIPGKLRAGFGALGIRPPPPGREESVEEFVRRNLGAEVFERLIEPFCSGVY
AGDPSKLSMKAAFGKVWRLEEAGGSIIGGTIKTIQERGKNPKPPRDPRLPTPKGQTVASFR
KGLAMLPNAITSSLGSKVKLSWKLTSITKSDGMGYVLVYETPEGVVSVQAKSVIMTIPSYVA
SDILRPLSSDAADALSRFYYPPVAAVTISYPKEAIRKECLIDGELQGFGQLHPRSQGVETLGT
IYSSSLFPNRAPAGRVLLLNYIGGAFNTGIVSKSASELVEAVDRDLRKMLINPSAVDPLVLGV
RVWPQAIPQFLVGHLDLLEAAKSSLDRGGYDGLFLGGNYVAGVALGRCVEGAYESASQIS
DFLTKYAYK*
Setaria italica PPO1 L427Y (without transit peptide) (SEQ ID NO: 333)
AAVAGGAAEAPASAGARVSADCVVVGGGISGLCTAQALATKHGVGDVLVTEARARPGGNI
TTVERPDEGYLWEEGPNSFQPSDPVLTMAVDSGLKDDLVFGDPNAPRFVLWEGKLRPVPS
KPADLPFFDLMSIPGKLRAGFGALGIRPPPPGREESVEEFVRRNLGAEVFERLIEPFCSGVY
AGDPSKLSMKAAFGKVWRLEEAGGSIIGGTIKTIQERGKNPKPPRDPRLPTPKGQTVASFR
KGLAMLPNAITSSLGSKVKLSWKLTSITKSDGMGYVLVYETPEGVVSVQAKSVIMTIPSYVA
SDILRPLSSDAADALSRFYYPPVAAVTISYPKEAIRKECLIDGELQGFGQLHPRSQGVETLGT
IYSSSLFPNRAPAGRVLLLNYIGGATNTGIVSKSASELVEAVDRDLRKMLINPSAVDPYVLGV
RVWPQAIPQFLVGHLDLLEAAKSSLDRGGYDGLFLGGNYVAGVALGRCVEGAYESASQIS
DFLTKYAYK*
Setaria italica PPO1 G444D (without transit peptide) (SEQ ID NO: 334)
AAVAGGAAEAPASAGARVSADCVVVGGGISGLCTAQALATKHGVGDVLVTEARARPGGNI
TTVERPDEGYLWEEGPNSFQPSDPVLTMAVDSGLKDDLVFGDPNAPRFVLWEGKLRPVPS
KPADLPFFDLMSIPGKLRAGFGALGIRPPPPGREESVEEFVRRNLGAEVFERLIEPFCSGVY
AGDPSKLSMKAAFGKVWRLEEAGGSIIGGTIKTIQERGKNPKPPRDPRLPTPKGQTVASFR
KGLAMLPNAITSSLGSKVKLSWKLTSITKSDGMGYVLVYETPEGVVSVQAKSVIMTIPSYVA
SDILRPLSSDAADALSRFYYPPVAAVTISYPKEAIRKECLIDGELQGFGQLHPRSQGVETLGT
IYSSSLFPNRAPAGRVLLLNYIGGATNTGIVSKSASELVEAVDRDLRKMLINPSAVDPLVLGV
RVWPQAIPQFLVDHLDLLEAAKSSLDRGGYDGLFLGGNYVAGVALGRCVEGAYESASQISD
FLTKYAYK*
Setaria italica PPO1 G444K (without transit peptide) (SEQ ID NO: 335)
AAVAGGAAEAPASAGARVSADCVVVGGGISGLCTAQALATKHGVGDVLVTEARARPGGNI
TTVERPDEGYLWEEGPNSFQPSDPVLTMAVDSGLKDDLVFGDPNAPRFVLWEGKLRPVPS
KPADLPFFDLMSIPGKLRAGFGALGIRPPPPGREESVEEFVRRNLGAEVFERLIEPFCSGVY
AGDPSKLSMKAAFGKVWRLEEAGGSIIGGTIKTIQERGKNPKPPRDPRLPTPKGQTVASFR
KGLAMLPNAITSSLGSKVKLSWKLTSITKSDGMGYVLVYETPEGVVSVQAKSVIMTIPSYVA
SDILRPLSSDAADALSRFYYPPVAAVTISYPKEAIRKECLIDGELQGFGQLHPRSQGVETLGT
IYSSSLFPNRAPAGRVLLLNYIGGATNTGIVSKSASELVEAVDRDLRKMLINPSAVDPLVLGV
RVWPQAIPQFLVKHLDLLEAAKSSLDRGGYDGLFLGGNYVAGVALGRCVEGAYESASQISD
FLTKYAYK*
Setaria italica PPO1 G444N (without transit peptide) (SEQ ID NO: 336)
AAVAGGAAEAPASAGARVSADCVVVGGGISGLCTAQALATKHGVGDVLVTEARARPGGNI
TTVERPDEGYLWEEGPNSFQPSDPVLTMAVDSGLKDDLVFGDPNAPRFVLWEGKLRPVPS
KPADLPFFDLMSIPGKLRAGFGALGIRPPPPGREESVEEFVRRNLGAEVFERLIEPFCSGVY
AGDPSKLSMKAAFGKVWRLEEAGGSIIGGTIKTIQERGKNPKPPRDPRLPTPKGQTVASFR
KGLAMLPNAITSSLGSKVKLSWKLTSITKSDGMGYVLVYETPEGVVSVQAKSVIMTIPSYVA
SDILRPLSSDAADALSRFYYPPVAAVTISYPKEAIRKECLIDGELQGFGQLHPRSQGVETLGT
IYSSSLFPNRAPAGRVLLLNYIGGATNTGIVSKSASELVEAVDRDLRKMLINPSAVDPLVLGV
RVWPQAIPQFLVNHLDLLEAAKSSLDRGGYDGLFLGGNYVAGVALGRCVEGAYESASQISD
FLTKYAYK*
S305 motif (SEQ ID NO: 337)
S; (K/N/R); (L/V); K; L; S; W; (E/K/T); L; L; (G/S); I; (I/K/S/T); K
Y361 motif (SEQ ID NO: 338)
L; (A/S); (I/K/N/Q/R/S); (F/L); (D/F/P/Q/R/T); Y; P; P; V; (A/G); (A/S); V
Y362 motif (SEQ ID NO: 339)
L; (A/S); (I/K/N/Q/R/S); (F/L); (D/F/H/Y); F; P; P; V; (A/G); (A/S); V
V365 motif (SEQ ID NO: 340)
L; (A/S); (I/K/N/Q/R/S); (F/L); (D/F/H/Y); Y; P; P; (L/M); (A/G); (A/S); V
G404 motif (SEQ ID NO: 341)
G; (I/L/V); E; T; L; A; (S/T); I; Y; S; S; S; L; F; P
Y426 motif (SEQ ID NO: 342)
L; F; P; (G/N); R; A; P; (A/D/K/N/P/S); G; (Q/R); (I/T/V); (M/L); (I/L); L;
(N/S); (C/L/M/T/V); I; G; G; (A/S); (K/L/T); N
T431 motif (SEQ ID NO: 343)
L; F; P; (G/N); R; A; P; (A/D/K/N/P/S); G; (Q/R); (I/T/V); (M/L); (I/L); L;
(N/S); (F/Y); I; G; G; (A/S); R; N
T461 motif (SEQ ID NO: 344)
(G/P/S); (D/K/N/R/S/T); (A/S); Q; (D/E); P; (F/L/R/S); (A/T/V); (L/T/V);
(G/S); V; (K/R); V; W; (P/R); (K/Q/R); A; I; P; Q; F; L; (I/V); G
L479 motif (SEQ ID NO: 345)
(G/P/S); (D/K/N/R/S/T); (A/S); (A/E/G/K/Q/V); (D/E); P; (F/L/R/S); (A/T/V);
(L/T/V); (G/S); V; (K/R); V; W; (P/R); (K/Q/R); A; I; P; Q; F; (M/N); (I/V); G
S305 motif 2 (SEQ ID NO: 346)
K; L; S; W; (E/K/T); L; L; (G/S); I
Y361 motif 2 (SEQ ID NO: 347)
(D/F/P/Q/R/T); Y; P; P; V; (A/G); (A/S); V
Y362 motif 2 (SEQ ID NO: 348)
(F/L); (D/F/H/Y); F; P; P; V; (A/G); (A/S); V
V365 motif 2 (SEQ ID NO: 349)
Y; P; P; (L/M); (A/G); (A/S); V
G404 motif 2 (SEQ ID NO: 350)
E; T; L; A; (S/T); I; Y; S; S; S; L; F; P
Y426 motif 2 (SEQ ID NO: 351)
L; (N/S); (C/L/M/T/V); I; G; G; (A/S); (K/L/T); N
T431 motif 2 (SEQ ID NO: 352)
L; (N/S); (F/Y); I; G; G; (A/S); R; N
T461 motif 2 (SEQ ID NO: 353)
(A/S); Q; (D/E); P; (F/L/R/S); (A/T/V); (L/T/V); (G/S); V; (K/R); V; W; (P/R);
(K/Q/R); A; I; P; Q; F; L
L479 motif 2 (SEQ ID NO: 354)
(G/P/S); (D/K/N/R/S/T); (A/S); (A/E/G/K/Q/V/T); (D/E); P; (F/L/R/S);
(A/T/V/K); (L/T/V); (G/S); V; (K/R); V; W; (P/R); (K/Q/R); A; I; P; Q; F;
(M/N); (I/V); G
L479 motif 3 (SEQ ID NO: 355)
AIPQF[M/N][I/V]G
AtPPO1 S305L Y362F G404A-DNA (SEQ ID NO: 356)
ATGGAGTTATCTCTTCTCCGTCCGACGACTCAATCGCTTCTTCCGTCGTTTTCGAA
GCCCAATCTCCGATTAAATGTTTATAAGCCTCTTAGACTCCGTTGTTCAGTGGCC
GGTGGACCAACCGTCGGATCTTCAAAAATCGAAGGCGGAGGAGGCACCACCATC
ACGACGGATTGTGTGATTGTCGGCGGAGGTATTAGTGGTCTTTGCATCGCTCAGG
CGCTTGCTACTAAGCATCCTGATGCTGCTCCGAATTTAATTGTGACCGAGGCTAA
GGATCGTGTTGGAGGCAACATTATCACTCGTGAAGAGAATGGTTTTCTCTGGGAA
GAAGGTCCCAATAGTTTTCAACCGTCTGATCCTATGCTCACTATGGTGGTAGATA
GTGGTTTGAAGGATGATTTGGTGTTGGGAGATCCTACTGCGCCAAGGTTTGTGTT
GTGGAATGGGAAATTGAGGCCGGTTCCATCGAAGCTAACAGACTTACCGTTCTTT
GATTTGATGAGTATTGGTGGGAAGATTAGAGCTGGTTTTGGTGCACTTGGCATTC
GACCGTCACCTCCAGGTCGTGAAGAATCTGTGGAGGAGTTTGTACGGCGTAACCT
CGGTGATGAGGTTTTTGAGCGCCTGATTGAACCGTTTTGTTCAGGTGTTTATGCTG
GTGATCCTTCAAAACTGAGCATGAAAGCAGCGTTTGGGAAGGTTTGGAAACTAG
AGCAAAATGGTGGAAGCATAATAGGTGGTACTTTTAAGGCAATTCAGGAGAGGA
AAAACGCTCCCAAGGCAGAACGAGACCCGCGCCTGCCAAAACCACAGGGCCAA
ACAGTTGGTTCTTTCAGGAAGGGACTTCGAATGTTGCCAGAAGCAATATCTGCAA
GATTAGGTAGCAAAGTTAAGTTGTCTTGGAAGCTCCTTGGTATCACTAAGCTGGA
GAGCGGAGGATACAACTTAACATATGAGACTCCAGATGGTTTAGTTTCCGTGCA
GAGCAAAAGTGTTGTAATGACGGTGCCATCTCATGTTGCAAGTGGTCTCTTGCGC
CCTCTTTCTGAATCTGCTGCAAATGCACTCTCAAAACTATATTTCCCACCAGTTGC
AGCAGTATCTATCTCGTACCCGAAAGAAGCAATCCGAACAGAATGTTTGATAGA
TGGTGAACTAAAGGGTTTTGGGCAATTGCATCCACGCACGCAAGGAGTTGAAAC
ATTAGCTACTATCTACAGCTCCTCACTCTTTCCAAATCGCGCACCGCCCGGAAGA
ATTTTGCTGTTGAACTACATTGGCGGGTCTACAAACACCGGAATTCTGTCCAAGT
CTGAAGGTGAGTTAGTGGAAGCAGTTGACAGAGATTTGAGGAAAATGCTAATTA
AGCCTAATTCGACCGATCCACTTAAATTAGGAGTTAGGGTATGGCCTCAAGCCAT
TCCTCAGTTTCTAGTTGGTCACTTTGATATCCTTGACACGGCTAAATCATCTCTAA
CGTCTTCGGGCTACGAAGGGCTATTTTTGGGTGGCAATTACGTCGCTGGTGTAGC
CTTAGGCCGGTGTGTAGAAGGCGCATATGAAACCGCGATTGAGGTCAACAACTT
CATGTCACGGTACGCTTACAAGTAA
AtPPO1 V365L G404A L479N-DNA (SEQ ID NO: 357)
ATGGAGTTATCTCTTCTCCGTCCGACGACTCAATCGCTTCTTCCGTCGTTTTCGAA
GCCCAATCTCCGATTAAATGTTTATAAGCCTCTTAGACTCCGTTGTTCAGTGGCC
GGTGGACCAACCGTCGGATCTTCAAAAATCGAAGGCGGAGGAGGCACCACCATC
ACGACGGATTGTGTGATTGTCGGCGGAGGTATTAGTGGTCTTTGCATCGCTCAGG
CGCTTGCTACTAAGCATCCTGATGCTGCTCCGAATTTAATTGTGACCGAGGCTAA
GGATCGTGTTGGAGGCAACATTATCACTCGTGAAGAGAATGGTTTTCTCTGGGAA
GAAGGTCCCAATAGTTTTCAACCGTCTGATCCTATGCTCACTATGGTGGTAGATA
GTGGTTTGAAGGATGATTTGGTGTTGGGAGATCCTACTGCGCCAAGGTTTGTGTT
GTGGAATGGGAAATTGAGGCCGGTTCCATCGAAGCTAACAGACTTACCGTTCTTT
GATTTGATGAGTATTGGTGGGAAGATTAGAGCTGGTTTTGGTGCACTTGGCATTC
GACCGTCACCTCCAGGTCGTGAAGAATCTGTGGAGGAGTTTGTACGGCGTAACCT
CGGTGATGAGGTTTTTGAGCGCCTGATTGAACCGTTTTGTTCAGGTGTTTATGCTG
GTGATCCTTCAAAACTGAGCATGAAAGCAGCGTTTGGGAAGGTTTGGAAACTAG
AGCAAAATGGTGGAAGCATAATAGGTGGTACTTTTAAGGCAATTCAGGAGAGGA
AAAACGCTCCCAAGGCAGAACGAGACCCGCGCCTGCCAAAACCACAGGGCCAA
ACAGTTGGTTCTTTCAGGAAGGGACTTCGAATGTTGCCAGAAGCAATATCTGCAA
GATTAGGTAGCAAAGTTAAGTTGTCTTGGAAGCTCTCAGGTATCACTAAGCTGGA
GAGCGGAGGATACAACTTAACATATGAGACTCCAGATGGTTTAGTTTCCGTGCA
GAGCAAAAGTGTTGTAATGACGGTGCCATCTCATGTTGCAAGTGGTCTCTTGCGC
CCTCTTTCTGAATCTGCTGCAAATGCACTCTCAAAACTATATTACCCACCACTTGC
AGCAGTATCTATCTCGTACCCGAAAGAAGCAATCCGAACAGAATGTTTGATAGA
TGGTGAACTAAAGGGTTTTGGGCAATTGCATCCACGCACGCAAGGAGTTGAAAC
ATTAGCTACTATCTACAGCTCCTCACTCTTTCCAAATCGCGCACCGCCCGGAAGA
ATTTTGCTGTTGAACTACATTGGCGGGTCTACAAACACCGGAATTCTGTCCAAGT
CTGAAGGTGAGTTAGTGGAAGCAGTTGACAGAGATTTGAGGAAAATGCTAATTA
AGCCTAATTCGACCGATCCACTTAAATTAGGAGTTAGGGTATGGCCTCAAGCCAT
TCCTCAGTTTAATGTTGGTCACTTTGATATCCTTGACACGGCTAAATCATCTCTAA
CGTCTTCGGGCTACGAAGGGCTATTTTTGGGTGGCAATTACGTCGCTGGTGTAGC
CTTAGGCCGGTGTGTAGAAGGCGCATATGAAACCGCGATTGAGGTCAACAACTT
CATGTCACGGTACGCTTACAAGTAA
AtPPO1 Y426M T461Q L479M-DNA (SEQ ID NO: 358)
ATGGAGTTATCTCTTCTCCGTCCGACGACTCAATCGCTTCTTCCGTCGTTTTCGAA
GCCCAATCTCCGATTAAATGTTTATAAGCCTCTTAGACTCCGTTGTTCAGTGGCC
GGTGGACCAACCGTCGGATCTTCAAAAATCGAAGGCGGAGGAGGCACCACCATC
ACGACGGATTGTGTGATTGTCGGCGGAGGTATTAGTGGTCTTTGCATCGCTCAGG
CGCTTGCTACTAAGCATCCTGATGCTGCTCCGAATTTAATTGTGACCGAGGCTAA
GGATCGTGTTGGAGGCAACATTATCACTCGTGAAGAGAATGGTTTTCTCTGGGAA
GAAGGTCCCAATAGTTTTCAACCGTCTGATCCTATGCTCACTATGGTGGTAGATA
GTGGTTTGAAGGATGATTTGGTGTTGGGAGATCCTACTGCGCCAAGGTTTGTGTT
GTGGAATGGGAAATTGAGGCCGGTTCCATCGAAGCTAACAGACTTACCGTTCTTT
GATTTGATGAGTATTGGTGGGAAGATTAGAGCTGGTTTTGGTGCACTTGGCATTC
GACCGTCACCTCCAGGTCGTGAAGAATCTGTGGAGGAGTTTGTACGGCGTAACCT
CGGTGATGAGGTTTTTGAGCGCCTGATTGAACCGTTTTGTTCAGGTGTTTATGCTG
GTGATCCTTCAAAACTGAGCATGAAAGCAGCGTTTGGGAAGGTTTGGAAACTAG
AGCAAAATGGTGGAAGCATAATAGGTGGTACTTTTAAGGCAATTCAGGAGAGGA
AAAACGCTCCCAAGGCAGAACGAGACCCGCGCCTGCCAAAACCACAGGGCCAA
ACAGTTGGTTCTTTCAGGAAGGGACTTCGAATGTTGCCAGAAGCAATATCTGCAA
GATTAGGTAGCAAAGTTAAGTTGTCTTGGAAGCTCTCAGGTATCACTAAGCTGGA
GAGCGGAGGATACAACTTAACATATGAGACTCCAGATGGTTTAGTTTCCGTGCA
GAGCAAAAGTGTTGTAATGACGGTGCCATCTCATGTTGCAAGTGGTCTCTTGCGC
CCTCTTTCTGAATCTGCTGCAAATGCACTCTCAAAACTATATTACCCACCAGTTGC
AGCAGTATCTATCTCGTACCCGAAAGAAGCAATCCGAACAGAATGTTTGATAGA
TGGTGAACTAAAGGGTTTTGGGCAATTGCATCCACGCACGCAAGGAGTTGAAAC
ATTAGGAACTATCTACAGCTCCTCACTCTTTCCAAATCGCGCACCGCCCGGAAGA
ATTTTGCTGTTGAACATGATTGGCGGGTCTACAAACACCGGAATTCTGTCCAAGT
CTGAAGGTGAGTTAGTGGAAGCAGTTGACAGAGATTTGAGGAAAATGCTAATTA
AGCCTAATTCGCAAGATCCACTTAAATTAGGAGTTAGGGTATGGCCTCAAGCCAT
TCCTCAGTTTATGGTTGGTCACTTTGATATCCTTGACACGGCTAAATCATCTCTAA
CGTCTTCGGGCTACGAAGGGCTATTTTTGGGTGGCAATTACGTCGCTGGTGTAGC
CTTAGGCCGGTGTGTAGAAGGCGCATATGAAACCGCGATTGAGGTCAACAACTT
CATGTCACGGTACGCTTACAAGTAA
AtPPO1 Y361D V365L-DNA (SEQ ID NO: 359)
ATGGAGTTATCTCTTCTCCGTCCGACGACTCAATCGCTTCTTCCGTCGTTTTCGAA
GCCCAATCTCCGATTAAATGTTTATAAGCCTCTTAGACTCCGTTGTTCAGTGGCC
GGTGGACCAACCGTCGGATCTTCAAAAATCGAAGGCGGAGGAGGCACCACCATC
ACGACGGATTGTGTGATTGTCGGCGGAGGTATTAGTGGTCTTTGCATCGCTCAGG
CGCTTGCTACTAAGCATCCTGATGCTGCTCCGAATTTAATTGTGACCGAGGCTAA
GGATCGTGTTGGAGGCAACATTATCACTCGTGAAGAGAATGGTTTTCTCTGGGAA
GAAGGTCCCAATAGTTTTCAACCGTCTGATCCTATGCTCACTATGGTGGTAGATA
GTGGTTTGAAGGATGATTTGGTGTTGGGAGATCCTACTGCGCCAAGGTTTGTGTT
GTGGAATGGGAAATTGAGGCCGGTTCCATCGAAGCTAACAGACTTACCGTTCTTT
GATTTGATGAGTATTGGTGGGAAGATTAGAGCTGGTTTTGGTGCACTTGGCATTC
GACCGTCACCTCCAGGTCGTGAAGAATCTGTGGAGGAGTTTGTACGGCGTAACCT
CGGTGATGAGGTTTTTGAGCGCCTGATTGAACCGTTTTGTTCAGGTGTTTATGCTG
GTGATCCTTCAAAACTGAGCATGAAAGCAGCGTTTGGGAAGGTTTGGAAACTAG
AGCAAAATGGTGGAAGCATAATAGGTGGTACTTTTAAGGCAATTCAGGAGAGGA
AAAACGCTCCCAAGGCAGAACGAGACCCGCGCCTGCCAAAACCACAGGGCCAA
ACAGTTGGTTCTTTCAGGAAGGGACTTCGAATGTTGCCAGAAGCAATATCTGCAA
GATTAGGTAGCAAAGTTAAGTTGTCTTGGAAGCTCTCAGGTATCACTAAGCTGGA
GAGCGGAGGATACAACTTAACATATGAGACTCCAGATGGTTTAGTTTCCGTGCA
GAGCAAAAGTGTTGTAATGACGGTGCCATCTCATGTTGCAAGTGGTCTCTTGCGC
CCTCTTTCTGAATCTGCTGCAAATGCACTCTCAAAACTAGATTACCCACCACTTG
CAGCAGTATCTATCTCGTACCCGAAAGAAGCAATCCGAACAGAATGTTTGATAG
ATGGTGAACTAAAGGGTTTTGGGCAATTGCATCCACGCACGCAAGGAGTTGAAA
CATTAGGAACTATCTACAGCTCCTCACTCTTTCCAAATCGCGCACCGCCCGGAAG
AATTTTGCTGTTGAACTACATTGGCGGGTCTACAAACACCGGAATTCTGTCCAAG
TCTGAAGGTGAGTTAGTGGAAGCAGTTGACAGAGATTTGAGGAAAATGCTAATT
AAGCCTAATTCGACCGATCCACTTAAATTAGGAGTTAGGGTATGGCCTCAAGCCA
TTCCTCAGTTTCTAGTTGGTCACTTTGATATCCTTGACACGGCTAAATCATCTCTA
ACGTCTTCGGGCTACGAAGGGCTATTTTTGGGTGGCAATTACGTCGCTGGTGTAG
CCTTAGGCCGGTGTGTAGAAGGCGCATATGAAACCGCGATTGAGGTCAACAACT
TCATGTCACGGTACGCTTACAAGTAA
AtPPO1 S305L G404A Y426V T431R L479N-DNA (SEQ ID NO: 360)
ATGGAGTTATCTCTTCTCCGTCCGACGACTCAATCGCTTCTTCCGTCGTTTTCGAA
GCCCAATCTCCGATTAAATGTTTATAAGCCTCTTAGACTCCGTTGTTCAGTGGCC
GGTGGACCAACCGTCGGATCTTCAAAAATCGAAGGCGGAGGAGGCACCACCATC
ACGACGGATTGTGTGATTGTCGGCGGAGGTATTAGTGGTCTTTGCATCGCTCAGG
CGCTTGCTACTAAGCATCCTGATGCTGCTCCGAATTTAATTGTGACCGAGGCTAA
GGATCGTGTTGGAGGCAACATTATCACTCGTGAAGAGAATGGTTTTCTCTGGGAA
GAAGGTCCCAATAGTTTTCAACCGTCTGATCCTATGCTCACTATGGTGGTAGATA
GTGGTTTGAAGGATGATTTGGTGTTGGGAGATCCTACTGCGCCAAGGTTTGTGTT
GTGGAATGGGAAATTGAGGCCGGTTCCATCGAAGCTAACAGACTTACCGTTCTTT
GATTTGATGAGTATTGGTGGGAAGATTAGAGCTGGTTTTGGTGCACTTGGCATTC
GACCGTCACCTCCAGGTCGTGAAGAATCTGTGGAGGAGTTTGTACGGCGTAACCT
CGGTGATGAGGTTTTTGAGCGCCTGATTGAACCGTTTTGTTCAGGTGTTTATGCTG
GTGATCCTTCAAAACTGAGCATGAAAGCAGCGTTTGGGAAGGTTTGGAAACTAG
AGCAAAATGGTGGAAGCATAATAGGTGGTACTTTTAAGGCAATTCAGGAGAGGA
AAAACGCTCCCAAGGCAGAACGAGACCCGCGCCTGCCAAAACCACAGGGCCAA
ACAGTTGGTTCTTTCAGGAAGGGACTTCGAATGTTGCCAGAAGCAATATCTGCAA
GATTAGGTAGCAAAGTTAAGTTGTCTTGGAAGCTCCTTGGTATCACTAAGCTGGA
GAGCGGAGGATACAACTTAACATATGAGACTCCAGATGGTTTAGTTTCCGTGCA
GAGCAAAAGTGTTGTAATGACGGTGCCATCTCATGTTGCAAGTGGTCTCTTGCGC
CCTCTTTCTGAATCTGCTGCAAATGCACTCTCAAAACTATATTACCCACCAGTTGC
AGCAGTATCTATCTCGTACCCGAAAGAAGCAATCCGAACAGAATGTTTGATAGA
TGGTGAACTAAAGGGTTTTGGGCAATTGCATCCACGCACGCAAGGAGTTGAAAC
ATTAGCTACTATCTACAGCTCCTCACTCTTTCCAAATCGCGCACCGCCCGGAAGA
ATTTTGCTGTTGAACGTTATTGGCGGGTCTAGAAACACCGGAATTCTGTCCAAGT
CTGAAGGTGAGTTAGTGGAAGCAGTTGACAGAGATTTGAGGAAAATGCTAATTA
AGCCTAATTCGACCGATCCACTTAAATTAGGAGTTAGGGTATGGCCTCAAGCCAT
TCCTCAGTTTAATGTTGGTCACTTTGATATCCTTGACACGGCTAAATCATCTCTAA
CGTCTTCGGGCTACGAAGGGCTATTTTTGGGTGGCAATTACGTCGCTGGTGTAGC
CTTAGGCCGGTGTGTAGAAGGCGCATATGAAACCGCGATTGAGGTCAACAACTT
CATGTCACGGTACGCTTACAAGTAA
AtPPO1 S305L V365M-DNA (SEQ ID NO: 361)
ATGGAGTTATCTCTTCTCCGTCCGACGACTCAATCGCTTCTTCCGTCGTTTTCGAA
GCCCAATCTCCGATTAAATGTTTATAAGCCTCTTAGACTCCGTTGTTCAGTGGCC
GGTGGACCAACCGTCGGATCTTCAAAAATCGAAGGCGGAGGAGGCACCACCATC
ACGACGGATTGTGTGATTGTCGGCGGAGGTATTAGTGGTCTTTGCATCGCTCAGG
CGCTTGCTACTAAGCATCCTGATGCTGCTCCGAATTTAATTGTGACCGAGGCTAA
GGATCGTGTTGGAGGCAACATTATCACTCGTGAAGAGAATGGTTTTCTCTGGGAA
GAAGGTCCCAATAGTTTTCAACCGTCTGATCCTATGCTCACTATGGTGGTAGATA
GTGGTTTGAAGGATGATTTGGTGTTGGGAGATCCTACTGCGCCAAGGTTTGTGTT
GTGGAATGGGAAATTGAGGCCGGTTCCATCGAAGCTAACAGACTTACCGTTCTTT
GATTTGATGAGTATTGGTGGGAAGATTAGAGCTGGTTTTGGTGCACTTGGCATTC
GACCGTCACCTCCAGGTCGTGAAGAATCTGTGGAGGAGTTTGTACGGCGTAACCT
CGGTGATGAGGTTTTTGAGCGCCTGATTGAACCGTTTTGTTCAGGTGTTTATGCTG
GTGATCCTTCAAAACTGAGCATGAAAGCAGCGTTTGGGAAGGTTTGGAAACTAG
AGCAAAATGGTGGAAGCATAATAGGTGGTACTTTTAAGGCAATTCAGGAGAGGA
AAAACGCTCCCAAGGCAGAACGAGACCCGCGCCTGCCAAAACCACAGGGCCAA
ACAGTTGGTTCTTTCAGGAAGGGACTTCGAATGTTGCCAGAAGCAATATCTGCAA
GATTAGGTAGCAAAGTTAAGTTGTCTTGGAAGCTCCTTGGTATCACTAAGCTGGA
GAGCGGAGGATACAACTTAACATATGAGACTCCAGATGGTTTAGTTTCCGTGCA
GAGCAAAAGTGTTGTAATGACGGTGCCATCTCATGTTGCAAGTGGTCTCTTGCGC
CCTCTTTCTGAATCTGCTGCAAATGCACTCTCAAAACTATATTACCCACCAATGG
CAGCAGTATCTATCTCGTACCCGAAAGAAGCAATCCGAACAGAATGTTTGATAG
ATGGTGAACTAAAGGGTTTTGGGCAATTGCATCCACGCACGCAAGGAGTTGAAA
CATTAGGAACTATCTACAGCTCCTCACTCTTTCCAAATCGCGCACCGCCCGGAAG
AATTTTGCTGTTGAACTACATTGGCGGGTCTACAAACACCGGAATTCTGTCCAAG
TCTGAAGGTGAGTTAGTGGAAGCAGTTGACAGAGATTTGAGGAAAATGCTAATT
AAGCCTAATTCGACCGATCCACTTAAATTAGGAGTTAGGGTATGGCCTCAAGCCA
TTCCTCAGTTTCTAGTTGGTCACTTTGATATCCTTGACACGGCTAAATCATCTCTA
ACGTCTTCGGGCTACGAAGGGCTATTTTTGGGTGGCAATTACGTCGCTGGTGTAG
CCTTAGGCCGGTGTGTAGAAGGCGCATATGAAACCGCGATTGAGGTCAACAACT
TCATGTCACGGTACGCTTACAAGTAA
AtPPO1 S305L G404A L479N-DNA (SEQ ID NO: 362)
ATGGAGTTATCTCTTCTCCGTCCGACGACTCAATCGCTTCTTCCGTCGTTTTCGAA
GCCCAATCTCCGATTAAATGTTTATAAGCCTCTTAGACTCCGTTGTTCAGTGGCC
GGTGGACCAACCGTCGGATCTTCAAAAATCGAAGGCGGAGGAGGCACCACCATC
ACGACGGATTGTGTGATTGTCGGCGGAGGTATTAGTGGTCTTTGCATCGCTCAGG
CGCTTGCTACTAAGCATCCTGATGCTGCTCCGAATTTAATTGTGACCGAGGCTAA
GGATCGTGTTGGAGGCAACATTATCACTCGTGAAGAGAATGGTTTTCTCTGGGAA
GAAGGTCCCAATAGTTTTCAACCGTCTGATCCTATGCTCACTATGGTGGTAGATA
GTGGTTTGAAGGATGATTTGGTGTTGGGAGATCCTACTGCGCCAAGGTTTGTGTT
GTGGAATGGGAAATTGAGGCCGGTTCCATCGAAGCTAACAGACTTACCGTTCTTT
GATTTGATGAGTATTGGTGGGAAGATTAGAGCTGGTTTTGGTGCACTTGGCATTC
GACCGTCACCTCCAGGTCGTGAAGAATCTGTGGAGGAGTTTGTACGGCGTAACCT
CGGTGATGAGGTTTTTGAGCGCCTGATTGAACCGTTTTGTTCAGGTGTTTATGCTG
GTGATCCTTCAAAACTGAGCATGAAAGCAGCGTTTGGGAAGGTTTGGAAACTAG
AGCAAAATGGTGGAAGCATAATAGGTGGTACTTTTAAGGCAATTCAGGAGAGGA
AAAACGCTCCCAAGGCAGAACGAGACCCGCGCCTGCCAAAACCACAGGGCCAA
ACAGTTGGTTCTTTCAGGAAGGGACTTCGAATGTTGCCAGAAGCAATATCTGCAA
GATTAGGTAGCAAAGTTAAGTTGTCTTGGAAGCTCCTTGGTATCACTAAGCTGGA
GAGCGGAGGATACAACTTAACATATGAGACTCCAGATGGTTTAGTTTCCGTGCA
GAGCAAAAGTGTTGTAATGACGGTGCCATCTCATGTTGCAAGTGGTCTCTTGCGC
CCTCTTTCTGAATCTGCTGCAAATGCACTCTCAAAACTATATTACCCACCAGTTGC
AGCAGTATCTATCTCGTACCCGAAAGAAGCAATCCGAACAGAATGTTTGATAGA
TGGTGAACTAAAGGGTTTTGGGCAATTGCATCCACGCACGCAAGGAGTTGAAAC
ATTAGCTACTATCTACAGCTCCTCACTCTTTCCAAATCGCGCACCGCCCGGAAGA
ATTTTGCTGTTGAACTACATTGGCGGGTCTACAAACACCGGAATTCTGTCCAAGT
CTGAAGGTGAGTTAGTGGAAGCAGTTGACAGAGATTTGAGGAAAATGCTAATTA
AGCCTAATTCGACCGATCCACTTAAATTAGGAGTTAGGGTATGGCCTCAAGCCAT
TCCTCAGTTTAATGTTGGTCACTTTGATATCCTTGACACGGCTAAATCATCTCTAA
CGTCTTCGGGCTACGAAGGGCTATTTTTGGGTGGCAATTACGTCGCTGGTGTAGC
CTTAGGCCGGTGTGTAGAAGGCGCATATGAAACCGCGATTGAGGTCAACAACTT
CATGTCACGGTACGCTTACAAGTAA
AtPPO1 S305L Y361T V365L T431R L479M-DNA (SEQ ID NO: 363)
ATGGAGTTATCTCTTCTCCGTCCGACGACTCAATCGCTTCTTCCGTCGTTTTCGAA
GCCCAATCTCCGATTAAATGTTTATAAGCCTCTTAGACTCCGTTGTTCAGTGGCC
GGTGGACCAACCGTCGGATCTTCAAAAATCGAAGGCGGAGGAGGCACCACCATC
ACGACGGATTGTGTGATTGTCGGCGGAGGTATTAGTGGTCTTTGCATCGCTCAGG
CGCTTGCTACTAAGCATCCTGATGCTGCTCCGAATTTAATTGTGACCGAGGCTAA
GGATCGTGTTGGAGGCAACATTATCACTCGTGAAGAGAATGGTTTTCTCTGGGAA
GAAGGTCCCAATAGTTTTCAACCGTCTGATCCTATGCTCACTATGGTGGTAGATA
GTGGTTTGAAGGATGATTTGGTGTTGGGAGATCCTACTGCGCCAAGGTTTGTGTT
GTGGAATGGGAAATTGAGGCCGGTTCCATCGAAGCTAACAGACTTACCGTTCTTT
GATTTGATGAGTATTGGTGGGAAGATTAGAGCTGGTTTTGGTGCACTTGGCATTC
GACCGTCACCTCCAGGTCGTGAAGAATCTGTGGAGGAGTTTGTACGGCGTAACCT
CGGTGATGAGGTTTTTGAGCGCCTGATTGAACCGTTTTGTTCAGGTGTTTATGCTG
GTGATCCTTCAAAACTGAGCATGAAAGCAGCGTTTGGGAAGGTTTGGAAACTAG
AGCAAAATGGTGGAAGCATAATAGGTGGTACTTTTAAGGCAATTCAGGAGAGGA
AAAACGCTCCCAAGGCAGAACGAGACCCGCGCCTGCCAAAACCACAGGGCCAA
ACAGTTGGTTCTTTCAGGAAGGGACTTCGAATGTTGCCAGAAGCAATATCTGCAA
GATTAGGTAGCAAAGTTAAGTTGTCTTGGAAGCTCCTTGGTATCACTAAGCTGGA
GAGCGGAGGATACAACTTAACATATGAGACTCCAGATGGTTTAGTTTCCGTGCA
GAGCAAAAGTGTTGTAATGACGGTGCCATCTCATGTTGCAAGTGGTCTCTTGCGC
CCTCTTTCTGAATCTGCTGCAAATGCACTCTCAAAACTAACTTACCCACCACTTGC
AGCAGTATCTATCTCGTACCCGAAAGAAGCAATCCGAACAGAATGTTTGATAGA
TGGTGAACTAAAGGGTTTTGGGCAATTGCATCCACGCACGCAAGGAGTTGAAAC
ATTAGGAACTATCTACAGCTCCTCACTCTTTCCAAATCGCGCACCGCCCGGAAGA
ATTTTGCTGTTGAACTACATTGGCGGGTCTAGAAACACCGGAATTCTGTCCAAGT
CTGAAGGTGAGTTAGTGGAAGCAGTTGACAGAGATTTGAGGAAAATGCTAATTA
AGCCTAATTCGACCGATCCACTTAAATTAGGAGTTAGGGTATGGCCTCAAGCCAT
TCCTCAGTTTATGGTTGGTCACTTTGATATCCTTGACACGGCTAAATCATCTCTAA
CGTCTTCGGGCTACGAAGGGCTATTTTTGGGTGGCAATTACGTCGCTGGTGTAGC
CTTAGGCCGGTGTGTAGAAGGCGCATATGAAACCGCGATTGAGGTCAACAACTT
CATGTCACGGTACGCTTACAAGTAA
AtPPO1 V365L Y426V L479N-DNA (SEQ ID NO: 364)
ATGGAGTTATCTCTTCTCCGTCCGACGACTCAATCGCTTCTTCCGTCGTTTTCGAA
GCCCAATCTCCGATTAAATGTTTATAAGCCTCTTAGACTCCGTTGTTCAGTGGCC
GGTGGACCAACCGTCGGATCTTCAAAAATCGAAGGCGGAGGAGGCACCACCATC
ACGACGGATTGTGTGATTGTCGGCGGAGGTATTAGTGGTCTTTGCATCGCTCAGG
CGCTTGCTACTAAGCATCCTGATGCTGCTCCGAATTTAATTGTGACCGAGGCTAA
GGATCGTGTTGGAGGCAACATTATCACTCGTGAAGAGAATGGTTTTCTCTGGGAA
GAAGGTCCCAATAGTTTTCAACCGTCTGATCCTATGCTCACTATGGTGGTAGATA
GTGGTTTGAAGGATGATTTGGTGTTGGGAGATCCTACTGCGCCAAGGTTTGTGTT
GTGGAATGGGAAATTGAGGCCGGTTCCATCGAAGCTAACAGACTTACCGTTCTTT
GATTTGATGAGTATTGGTGGGAAGATTAGAGCTGGTTTTGGTGCACTTGGCATTC
GACCGTCACCTCCAGGTCGTGAAGAATCTGTGGAGGAGTTTGTACGGCGTAACCT
CGGTGATGAGGTTTTTGAGCGCCTGATTGAACCGTTTTGTTCAGGTGTTTATGCTG
GTGATCCTTCAAAACTGAGCATGAAAGCAGCGTTTGGGAAGGTTTGGAAACTAG
AGCAAAATGGTGGAAGCATAATAGGTGGTACTTTTAAGGCAATTCAGGAGAGGA
AAAACGCTCCCAAGGCAGAACGAGACCCGCGCCTGCCAAAACCACAGGGCCAA
ACAGTTGGTTCTTTCAGGAAGGGACTTCGAATGTTGCCAGAAGCAATATCTGCAA
GATTAGGTAGCAAAGTTAAGTTGTCTTGGAAGCTCTCAGGTATCACTAAGCTGGA
GAGCGGAGGATACAACTTAACATATGAGACTCCAGATGGTTTAGTTTCCGTGCA
GAGCAAAAGTGTTGTAATGACGGTGCCATCTCATGTTGCAAGTGGTCTCTTGCGC
CCTCTTTCTGAATCTGCTGCAAATGCACTCTCAAAACTATATTACCCACCACTTGC
AGCAGTATCTATCTCGTACCCGAAAGAAGCAATCCGAACAGAATGTTTGATAGA
TGGTGAACTAAAGGGTTTTGGGCAATTGCATCCACGCACGCAAGGAGTTGAAAC
ATTAGGAACTATCTACAGCTCCTCACTCTTTCCAAATCGCGCACCGCCCGGAAGA
ATTTTGCTGTTGAACGTTATTGGCGGGTCTACAAACACCGGAATTCTGTCCAAGT
CTGAAGGTGAGTTAGTGGAAGCAGTTGACAGAGATTTGAGGAAAATGCTAATTA
AGCCTAATTCGACCGATCCACTTAAATTAGGAGTTAGGGTATGGCCTCAAGCCAT
TCCTCAGTTTAATGTTGGTCACTTTGATATCCTTGACACGGCTAAATCATCTCTAA
CGTCTTCGGGCTACGAAGGGCTATTTTTGGGTGGCAATTACGTCGCTGGTGTAGC
CTTAGGCCGGTGTGTAGAAGGCGCATATGAAACCGCGATTGAGGTCAACAACTT
CATGTCACGGTACGCTTACAAGTAA
AtPPO1 S305L Y426V L479M-DNA (SEQ ID NO: 365)
ATGGAGTTATCTCTTCTCCGTCCGACGACTCAATCGCTTCTTCCGTCGTTTTCGAA
GCCCAATCTCCGATTAAATGTTTATAAGCCTCTTAGACTCCGTTGTTCAGTGGCC
GGTGGACCAACCGTCGGATCTTCAAAAATCGAAGGCGGAGGAGGCACCACCATC
ACGACGGATTGTGTGATTGTCGGCGGAGGTATTAGTGGTCTTTGCATCGCTCAGG
CGCTTGCTACTAAGCATCCTGATGCTGCTCCGAATTTAATTGTGACCGAGGCTAA
GGATCGTGTTGGAGGCAACATTATCACTCGTGAAGAGAATGGTTTTCTCTGGGAA
GAAGGTCCCAATAGTTTTCAACCGTCTGATCCTATGCTCACTATGGTGGTAGATA
GTGGTTTGAAGGATGATTTGGTGTTGGGAGATCCTACTGCGCCAAGGTTTGTGTT
GTGGAATGGGAAATTGAGGCCGGTTCCATCGAAGCTAACAGACTTACCGTTCTTT
GATTTGATGAGTATTGGTGGGAAGATTAGAGCTGGTTTTGGTGCACTTGGCATTC
GACCGTCACCTCCAGGTCGTGAAGAATCTGTGGAGGAGTTTGTACGGCGTAACCT
CGGTGATGAGGTTTTTGAGCGCCTGATTGAACCGTTTTGTTCAGGTGTTTATGCTG
GTGATCCTTCAAAACTGAGCATGAAAGCAGCGTTTGGGAAGGTTTGGAAACTAG
AGCAAAATGGTGGAAGCATAATAGGTGGTACTTTTAAGGCAATTCAGGAGAGGA
AAAACGCTCCCAAGGCAGAACGAGACCCGCGCCTGCCAAAACCACAGGGCCAA
ACAGTTGGTTCTTTCAGGAAGGGACTTCGAATGTTGCCAGAAGCAATATCTGCAA
GATTAGGTAGCAAAGTTAAGTTGTCTTGGAAGCTCCTTGGTATCACTAAGCTGGA
GAGCGGAGGATACAACTTAACATATGAGACTCCAGATGGTTTAGTTTCCGTGCA
GAGCAAAAGTGTTGTAATGACGGTGCCATCTCATGTTGCAAGTGGTCTCTTGCGC
CCTCTTTCTGAATCTGCTGCAAATGCACTCTCAAAACTATATTACCCACCAGTTGC
AGCAGTATCTATCTCGTACCCGAAAGAAGCAATCCGAACAGAATGTTTGATAGA
TGGTGAACTAAAGGGTTTTGGGCAATTGCATCCACGCACGCAAGGAGTTGAAAC
ATTAGGAACTATCTACAGCTCCTCACTCTTTCCAAATCGCGCACCGCCCGGAAGA
ATTTTGCTGTTGAACGTTATTGGCGGGTCTACAAACACCGGAATTCTGTCCAAGT
CTGAAGGTGAGTTAGTGGAAGCAGTTGACAGAGATTTGAGGAAAATGCTAATTA
AGCCTAATTCGACCGATCCACTTAAATTAGGAGTTAGGGTATGGCCTCAAGCCAT
TCCTCAGTTTATGGTTGGTCACTTTGATATCCTTGACACGGCTAAATCATCTCTAA
CGTCTTCGGGCTACGAAGGGCTATTTTTGGGTGGCAATTACGTCGCTGGTGTAGC
CTTAGGCCGGTGTGTAGAAGGCGCATATGAAACCGCGATTGAGGTCAACAACTT
CATGTCACGGTACGCTTACAAGTAA
AtPPO1 S305L Y426V-DNA (SEQ ID NO: 366)
ATGGAGTTATCTCTTCTCCGTCCGACGACTCAATCGCTTCTTCCGTCGTTTTCGAA
GCCCAATCTCCGATTAAATGTTTATAAGCCTCTTAGACTCCGTTGTTCAGTGGCC
GGTGGACCAACCGTCGGATCTTCAAAAATCGAAGGCGGAGGAGGCACCACCATC
ACGACGGATTGTGTGATTGTCGGCGGAGGTATTAGTGGTCTTTGCATCGCTCAGG
CGCTTGCTACTAAGCATCCTGATGCTGCTCCGAATTTAATTGTGACCGAGGCTAA
GGATCGTGTTGGAGGCAACATTATCACTCGTGAAGAGAATGGTTTTCTCTGGGAA
GAAGGTCCCAATAGTTTTCAACCGTCTGATCCTATGCTCACTATGGTGGTAGATA
GTGGTTTGAAGGATGATTTGGTGTTGGGAGATCCTACTGCGCCAAGGTTTGTGTT
GTGGAATGGGAAATTGAGGCCGGTTCCATCGAAGCTAACAGACTTACCGTTCTTT
GATTTGATGAGTATTGGTGGGAAGATTAGAGCTGGTTTTGGTGCACTTGGCATTC
GACCGTCACCTCCAGGTCGTGAAGAATCTGTGGAGGAGTTTGTACGGCGTAACCT
CGGTGATGAGGTTTTTGAGCGCCTGATTGAACCGTTTTGTTCAGGTGTTTATGCTG
GTGATCCTTCAAAACTGAGCATGAAAGCAGCGTTTGGGAAGGTTTGGAAACTAG
AGCAAAATGGTGGAAGCATAATAGGTGGTACTTTTAAGGCAATTCAGGAGAGGA
AAAACGCTCCCAAGGCAGAACGAGACCCGCGCCTGCCAAAACCACAGGGCCAA
ACAGTTGGTTCTTTCAGGAAGGGACTTCGAATGTTGCCAGAAGCAATATCTGCAA
GATTAGGTAGCAAAGTTAAGTTGTCTTGGAAGCTCCTTGGTATCACTAAGCTGGA
GAGCGGAGGATACAACTTAACATATGAGACTCCAGATGGTTTAGTTTCCGTGCA
GAGCAAAAGTGTTGTAATGACGGTGCCATCTCATGTTGCAAGTGGTCTCTTGCGC
CCTCTTTCTGAATCTGCTGCAAATGCACTCTCAAAACTATATTACCCACCAGTTGC
AGCAGTATCTATCTCGTACCCGAAAGAAGCAATCCGAACAGAATGTTTGATAGA
TGGTGAACTAAAGGGTTTTGGGCAATTGCATCCACGCACGCAAGGAGTTGAAAC
ATTAGGAACTATCTACAGCTCCTCACTCTTTCCAAATCGCGCACCGCCCGGAAGA
ATTTTGCTGTTGAACGTTATTGGCGGGTCTACAAACACCGGAATTCTGTCCAAGT
CTGAAGGTGAGTTAGTGGAAGCAGTTGACAGAGATTTGAGGAAAATGCTAATTA
AGCCTAATTCGACCGATCCACTTAAATTAGGAGTTAGGGTATGGCCTCAAGCCAT
TCCTCAGTTTCTAGTTGGTCACTTTGATATCCTTGACACGGCTAAATCATCTCTAA
CGTCTTCGGGCTACGAAGGGCTATTTTTGGGTGGCAATTACGTCGCTGGTGTAGC
CTTAGGCCGGTGTGTAGAAGGCGCATATGAAACCGCGATTGAGGTCAACAACTT
CATGTCACGGTACGCTTACAAGTAA
AtPPO1 S305L Y361C Y362F Y426M L479M v31-DNA (SEQ ID NO: 367)
ATGGAGTTATCTCTTCTCCGTCCGACGACTCAATCGCTTCTTCCGTCGTTTTCGAA
GCCCAATCTCCGATTAAATGTTTATAAGCCTCTTAGACTCCGTTGTTCAGTGGCC
GGTGGACCAACCGTCGGATCTTCAAAAATCGAAGGCGGAGGAGGCACCACCATC
ACGACGGATTGTGTGATTGTCGGCGGAGGTATTAGTGGTCTTTGCATCGCTCAGG
CGCTTGCTACTAAGCATCCTGATGCTGCTCCGAATTTAATTGTGACCGAGGCTAA
GGATCGTGTTGGAGGCAACATTATCACTCGTGAAGAGAATGGTTTTCTCTGGGAA
GAAGGTCCCAATAGTTTTCAACCGTCTGATCCTATGCTCACTATGGTGGTAGATA
GTGGTTTGAAGGATGATTTGGTGTTGGGAGATCCTACTGCGCCAAGGTTTGTGTT
GTGGAATGGGAAATTGAGGCCGGTTCCATCGAAGCTAACAGACTTACCGTTCTTT
GATTTGATGAGTATTGGTGGGAAGATTAGAGCTGGTTTTGGTGCACTTGGCATTC
GACCGTCACCTCCAGGTCGTGAAGAATCTGTGGAGGAGTTTGTACGGCGTAACCT
CGGTGATGAGGTTTTTGAGCGCCTGATTGAACCGTTTTGTTCAGGTGTTTATGCTG
GTGATCCTTCAAAACTGAGCATGAAAGCAGCGTTTGGGAAGGTTTGGAAACTAG
AGCAAAATGGTGGAAGCATAATAGGTGGTACTTTTAAGGCAATTCAGGAGAGGA
AAAACGCTCCCAAGGCAGAACGAGACCCGCGCCTGCCAAAACCACAGGGCCAA
ACAGTTGGTTCTTTCAGGAAGGGACTTCGAATGTTGCCAGAAGCAATATCTGCAA
GATTAGGTAGCAAAGTTAAGTTGTCTTGGAAGCTCCTTGGTATCACTAAGCTGGA
GAGCGGAGGATACAACTTAACATATGAGACTCCAGATGGTTTAGTTTCCGTGCA
GAGCAAAAGTGTTGTAATGACGGTGCCATCTCATGTTGCAAGTGGTCTCTTGCGC
CCTCTTTCTGAATCTGCTGCAAATGCACTCTCAAAACTATGTTTCCCACCAGTTGC
AGCAGTATCTATCTCGTACCCGAAAGAAGCAATCCGAACAGAATGTTTGATAGA
TGGTGAACTAAAGGGTTTTGGGCAATTGCATCCACGCACGCAAGGAGTTGAAAC
ATTAGGAACTATCTACAGCTCCTCACTCTTTCCAAATCGCGCACCGCCCGGAAGA
ATTTTGCTGTTGAACATGATTGGCGGGTCTACAAACACCGGAATTCTGTCCAAGT
CTGAAGGTGAGTTAGTGGAAGCAGTTGACAGAGATTTGAGGAAAATGCTAATTA
AGCCTAATTCGACCGATCCACTTAAATTAGGAGTTAGGGTATGGCCTCAAGCCAT
TCCTCAGTTTATGGTTGGTCACTTTGATATCCTTGACACGGCTAAATCATCTCTAA
CGTCTTCGGGCTACGAAGGGCTATTTTTGGGTGGCAATTACGTCGCTGGTGTAGC
CTTAGGCCGGTGTGTAGAAGGCGCATATGAAACCGCGATTGAGGTCAACAACTT
CATGTCACGGTACGCTTACAAGTAA
AtPPO1 S305L Y361D V365M-DNA (SEQ ID NO: 368)
ATGGAGTTATCTCTTCTCCGTCCGACGACTCAATCGCTTCTTCCGTCGTTTTCGAA
GCCCAATCTCCGATTAAATGTTTATAAGCCTCTTAGACTCCGTTGTTCAGTGGCC
GGTGGACCAACCGTCGGATCTTCAAAAATCGAAGGCGGAGGAGGCACCACCATC
ACGACGGATTGTGTGATTGTCGGCGGAGGTATTAGTGGTCTTTGCATCGCTCAGG
CGCTTGCTACTAAGCATCCTGATGCTGCTCCGAATTTAATTGTGACCGAGGCTAA
GGATCGTGTTGGAGGCAACATTATCACTCGTGAAGAGAATGGTTTTCTCTGGGAA
GAAGGTCCCAATAGTTTTCAACCGTCTGATCCTATGCTCACTATGGTGGTAGATA
GTGGTTTGAAGGATGATTTGGTGTTGGGAGATCCTACTGCGCCAAGGTTTGTGTT
GTGGAATGGGAAATTGAGGCCGGTTCCATCGAAGCTAACAGACTTACCGTTCTTT
GATTTGATGAGTATTGGTGGGAAGATTAGAGCTGGTTTTGGTGCACTTGGCATTC
GACCGTCACCTCCAGGTCGTGAAGAATCTGTGGAGGAGTTTGTACGGCGTAACCT
CGGTGATGAGGTTTTTGAGCGCCTGATTGAACCGTTTTGTTCAGGTGTTTATGCTG
GTGATCCTTCAAAACTGAGCATGAAAGCAGCGTTTGGGAAGGTTTGGAAACTAG
AGCAAAATGGTGGAAGCATAATAGGTGGTACTTTTAAGGCAATTCAGGAGAGGA
AAAACGCTCCCAAGGCAGAACGAGACCCGCGCCTGCCAAAACCACAGGGCCAA
ACAGTTGGTTCTTTCAGGAAGGGACTTCGAATGTTGCCAGAAGCAATATCTGCAA
GATTAGGTAGCAAAGTTAAGTTGTCTTGGAAGCTCCTTGGTATCACTAAGCTGGA
GAGCGGAGGATACAACTTAACATATGAGACTCCAGATGGTTTAGTTTCCGTGCA
GAGCAAAAGTGTTGTAATGACGGTGCCATCTCATGTTGCAAGTGGTCTCTTGCGC
CCTCTTTCTGAATCTGCTGCAAATGCACTCTCAAAACTAGATTACCCACCAATGG
CAGCAGTATCTATCTCGTACCCGAAAGAAGCAATCCGAACAGAATGTTTGATAG
ATGGTGAACTAAAGGGTTTTGGGCAATTGCATCCACGCACGCAAGGAGTTGAAA
CATTAGGAACTATCTACAGCTCCTCACTCTTTCCAAATCGCGCACCGCCCGGAAG
AATTTTGCTGTTGAACTACATTGGCGGGTCTACAAACACCGGAATTCTGTCCAAG
TCTGAAGGTGAGTTAGTGGAAGCAGTTGACAGAGATTTGAGGAAAATGCTAATT
AAGCCTAATTCGACCGATCCACTTAAATTAGGAGTTAGGGTATGGCCTCAAGCCA
TTCCTCAGTTTCTAGTTGGTCACTTTGATATCCTTGACACGGCTAAATCATCTCTA
ACGTCTTCGGGCTACGAAGGGCTATTTTTGGGTGGCAATTACGTCGCTGGTGTAG
CCTTAGGCCGGTGTGTAGAAGGCGCATATGAAACCGCGATTGAGGTCAACAACT
TCATGTCACGGTACGCTTACAAGTAA
AtPPO1-DNA (SEQ ID NO: 369)
ATGGAGTTATCTCTTCTCCGTCCGACGACTCAATCGCTTCTTCCGTCGTTTTCGAA
GCCCAATCTCCGATTAAATGTTTATAAGCCTCTTAGACTCCGTTGTTCAGTGGCC
GGTGGACCAACCGTCGGATCTTCAAAAATCGAAGGCGGAGGAGGCACCACCATC
ACGACGGATTGTGTGATTGTCGGCGGAGGTATTAGTGGTCTTTGCATCGCTCAGG
CGCTTGCTACTAAGCATCCTGATGCTGCTCCGAATTTAATTGTGACCGAGGCTAA
GGATCGTGTTGGAGGCAACATTATCACTCGTGAAGAGAATGGTTTTCTCTGGGAA
GAAGGTCCCAATAGTTTTCAACCGTCTGATCCTATGCTCACTATGGTGGTAGATA
GTGGTTTGAAGGATGATTTGGTGTTGGGAGATCCTACTGCGCCAAGGTTTGTGTT
GTGGAATGGGAAATTGAGGCCGGTTCCATCGAAGCTAACAGACTTACCGTTCTTT
GATTTGATGAGTATTGGTGGGAAGATTAGAGCTGGTTTTGGTGCACTTGGCATTC
GACCGTCACCTCCAGGTCGTGAAGAATCTGTGGAGGAGTTTGTACGGCGTAACCT
CGGTGATGAGGTTTTTGAGCGCCTGATTGAACCGTTTTGTTCAGGTGTTTATGCTG
GTGATCCTTCAAAACTGAGCATGAAAGCAGCGTTTGGGAAGGTTTGGAAACTAG
AGCAAAATGGTGGAAGCATAATAGGTGGTACTTTTAAGGCAATTCAGGAGAGGA
AAAACGCTCCCAAGGCAGAACGAGACCCGCGCCTGCCAAAACCACAGGGCCAA
ACAGTTGGTTCTTTCAGGAAGGGACTTCGAATGTTGCCAGAAGCAATATCTGCAA
GATTAGGTAGCAAAGTTAAGTTGTCTTGGAAGCTCTCAGGTATCACTAAGCTGGA
GAGCGGAGGATACAACTTAACATATGAGACTCCAGATGGTTTAGTTTCCGTGCA
GAGCAAAAGTGTTGTAATGACGGTGCCATCTCATGTTGCAAGTGGTCTCTTGCGC
CCTCTTTCTGAATCTGCTGCAAATGCACTCTCAAAACTATATTACCCACCAGTTGC
AGCAGTATCTATCTCGTACCCGAAAGAAGCAATCCGAACAGAATGTTTGATAGA
TGGTGAACTAAAGGGTTTTGGGCAATTGCATCCACGCACGCAAGGAGTTGAAAC
ATTAGGAACTATCTACAGCTCCTCACTCTTTCCAAATCGCGCACCGCCCGGAAGA
ATTTTGCTGTTGAACTACATTGGCGGGTCTACAAACACCGGAATTCTGTCCAAGT
CTGAAGGTGAGTTAGTGGAAGCAGTTGACAGAGATTTGAGGAAAATGCTAATTA
AGCCTAATTCGACCGATCCACTTAAATTAGGAGTTAGGGTATGGCCTCAAGCCAT
TCCTCAGTTTCTAGTTGGTCACTTTGATATCCTTGACACGGCTAAATCATCTCTAA
CGTCTTCGGGCTACGAAGGGCTATTTTTGGGTGGCAATTACGTCGCTGGTGTAGC
CTTAGGCCGGTGTGTAGAAGGCGCATATGAAACCGCGATTGAGGTCAACAACTT
CATGTCACGGTACGCTTACAAGTAA
His-TEV-AtPPO1 ΔcTP WT (SEQ ID NO: 370)
MGSSHHHHHHSSGENLYFQSVAGGPTVGSSKIEGGGGTTITTDCVIVGGGISGLCIAQ
ALATKHPDAAPNLIVTEAKDRVGGNIITREENGFLWEEGPNSFQPSDPMLTMVVDSG
LKDDLVLGDPTAPRFVLWNGKLRPVPSKLTDLPFFDLMSIGGKIRAGFGALGIRPSPP
GREESVEEFVRRNLGDEVFERLIEPFCSGVYAGDPSKLSMKAAFGKVWKLEQNGGSI
IGGTFKAIQERKNAPKAERDPRLPKPQGQTVGSFRKGLRMLPEAISARLGSKVKLSW
KLSGITKLESGGYNLTYETPDGLVSVQSKSVVMTVPSHVASGLLRPLSESAANALSK
LYYPPVAAVSISYPKEAIRTECLIDGELKGFGQLHPRTQGVETLGTIYSSSLFPNRAPP
GRILLLNYIGGSTNTGILSKSEGELVEAVDRDLRKMLIKPNSTDPLKLGVRVWPQAIP
QFLVGHFDILDTAKSSLTSSGYEGLFLGGNYVAGVALGRCVEGAYETAIEVNNFMSR
YAYK*
His-TEV-AtPPO1 ΔcTP L479M (SEQ ID NO: 371)
MGSSHHHHHHSSGENLYFQSVAGGPTVGSSKIEGGGGTTITTDCVIVGGGISGLCIAQ
ALATKHPDAAPNLIVTEAKDRVGGNIITREENGFLWEEGPNSFQPSDPMLTMVVDSG
LKDDLVLGDPTAPRFVLWNGKLRPVPSKLTDLPFFDLMSIGGKIRAGFGALGIRPSPP
GREESVEEFVRRNLGDEVFERLIEPFCSGVYAGDPSKLSMKAAFGKVWKLEQNGGSI
IGGTFKAIQERKNAPKAERDPRLPKPQGQTVGSFRKGLRMLPEAISARLGSKVKLSW
KLSGITKLESGGYNLTYETPDGLVSVQSKSVVMTVPSHVASGLLRPLSESAANALSK
LYYPPVAAVSISYPKEAIRTECLIDGELKGFGQLHPRTQGVETLGTIYSSSLFPNRAPP
GRILLLNYIGGSTNTGILSKSEGELVEAVDRDLRKMLIKPNSTDPLKLGVRVWPQAIP
QFMVGHFDILDTAKSSLTSSGYEGLFLGGNYVAGVALGRCVEGAYETAIEVNNFMS
RYAYK*
His-TEV-AtPPO1 ΔcTP L479N (SEQ ID NO: 372)
MGSSHHHHHHSSGENLYFQSVAGGPTVGSSKIEGGGGTTITTDCVIVGGGISGLCIAQ
ALATKHPDAAPNLIVTEAKDRVGGNIITREENGFLWEEGPNSFQPSDPMLTMVVDSG
LKDDLVLGDPTAPRFVLWNGKLRPVPSKLTDLPFFDLMSIGGKIRAGFGALGIRPSPP
GREESVEEFVRRNLGDEVFERLIEPFCSGVYAGDPSKLSMKAAFGKVWKLEQNGGSI
IGGTFKAIQERKNAPKAERDPRLPKPQGQTVGSFRKGLRMLPEAISARLGSKVKLSW
KLSGITKLESGGYNLTYETPDGLVSVQSKSVVMTVPSHVASGLLRPLSESAANALSK
LYYPPVAAVSISYPKEAIRTECLIDGELKGFGQLHPRTQGVETLGTIYSSSLFPNRAPP
GRILLLNYIGGSTNTGILSKSEGELVEAVDRDLRKMLIKPNSTDPLKLGVRVWPQAIP
QFNVGHFDILDTAKSSLTSSGYEGLFLGGNYVAGVALGRCVEGAYETAIEVNNFMSR
YAYK*
His-TEV-AtPPO1 ΔcTP V002 (SEQ ID NO: 373)
MGSSHHHHHHSSGENLYFQSVAGGPTVGSSKIEGGGGTTITTDCVIVGGGISGLCIAQ
ALATKHPDAAPNLIVTEAKDRVGGNIITREENGFLWEEGPNSFQPSDPMLTMVVDSG
LKDDLVLGDPTAPRFVLWNGKLRPVPSKLTDLPFFDLMSIGGKIRAGFGALGIRPSPP
GREESVEEFVRRNLGDEVFERLIEPFCSGVYAGDPSKLSMKAAFGKVWKLEQNGGSI
IGGTFKAIQERKNAPKAERDPRLPKPQGQTVGSFRKGLRMLPEAISARLGSKVKLSW
KLLGITKLESGGYNLTYETPDGLVSVQSKSVVMTVPSHVASGLLRPLSESAANALSK
LYYPPVAAVSISYPKEAIRTECLIDGELKGFGQLHPRTQGVETLGTIYSSSLFPNRAPP
GRILLLNVIGGSTNTGILSKSEGELVEAVDRDLRKMLIKPNSTDPLKLGVRVWPQAIP
QFMVGHFDILDTAKSSLTSSGYEGLFLGGNYVAGVALGRCVEGAYETAIEVNNFMS
RYAYK*
His-TEV-AtPPO1 ΔcTP V030 (SEQ ID NO: 374)
MGSSHHHHHHSSGENLYFQSVAGGPTVGSSKIEGGGGTTITTDCVIVGGGISGLCIAQ
ALATKHPDAAPNLIVTEAKDRVGGNIITREENGFLWEEGPNSFQPSDPMLTMVVDSG
LKDDLVLGDPTAPRFVLWNGKLRPVPSKLTDLPFFDLMSIGGKIRAGFGALGIRPSPP
GREESVEEFVRRNLGDEVFERLIEPFCSGVYAGDPSKLSMKAAFGKVWKLEQNGGSI
IGGTFKAIQERKNAPKAERDPRLPKPQGQTVGSFRKGLRMLPEAISARLGSKVKLSW
KLLGITKLESGGYNLTYETPDGLVSVQSKSVVMTVPSHVASGLLRPLSESAANALSK
LYYPPVAAVSISYPKEAIRTECLIDGELKGFGQLHPRTQGVETLGTIYSSSLFPNRAPP
GRILLLNVIGGSTNTGILSKSEGELVEAVDRDLRKMLIKPNSTDPLKLGVRVWPQAIP
QFLVGHFDILDTAKSSLTSSGYEGLFLGGNYVAGVALGRCVEGAYETAIEVNNFMSR
YAYK*
His Tag (SEQ ID NO: 375)
HHHHHH
TEV protease recognition (SEQ ID NO: 376)
ENLYFQS
His-TEV (SEQ ID NO: 377)
HHHHHHENLYFQS
His-TEV 2 (SEQ ID NO: 378)
MGSSHHHHHHSSGENLYFQS

1. A modified Protoporphyrinogen Oxidase (PPO) enzyme or functional fragment thereof, wherein the modified Protoporphyrinogen Oxidase or functional fragment thereof comprises at least one mutation at one or more amino acid residues selected from residues 479, 365 and/or 362 of SEQ ID NO:1, or residues corresponding thereto.

2. The modified PPO enzyme or functional fragment thereof of claim 1, wherein the modified Protoporphyrinogen Oxidase or functional fragment thereof comprises the motif:
(SEQ ID NO: 354)
(G/P/S); (D/K/N/R/S/T); (A/S); (A/E/G/K/Q/V/T);
(D/E); P; (F/L/R/S); (A/T/V/K); (L/T/V); (G/S);
V; (K/R); V; W; (P/R); (K/Q/R); A; I; P; Q; F;
(M/N); (I/V); G;
or
(SEQ ID NO: 356)
AIPQF[M/N][I/V]G

3. The modified PPO enzyme or functional fragment thereof of claim 1, comprising a combination of mutations at residues:
(a) 365, 426, and 479 of SEQ ID NO:1, or residues corresponding thereto; optionally wherein the mutations comprise substitutions of 365L, 426V and 479N of SEQ ID NO:1, or substitutions corresponding thereto;
(b) 305, 426 and 479 of SEQ ID NO:1, or residues corresponding thereto; optionally wherein the mutations comprise substitutions of 305L, 426V and 479M of SEQ ID NO:1, or substitutions corresponding thereto; or
(c) 305, 361, 362, 426, and 479 of SEQ ID NO:1, or residues corresponding thereto;
optionally wherein the mutations comprise substitutions of 305L, 361C, 362F, 426M and 479M of SEQ ID NO: 1, or substitutions corresponding thereto.

4. The modified PPO enzyme or functional fragment thereof of claim 1, wherein the modified PPO enzyme or functional fragment thereof comprises an amino acid sequence having at least 30%, at least 35%, at least 40%, at least 45%, at least 50%, at least 55%, at least 60%, at least 70%, at least 75%, at least 80%, at least 85%, at least 90%, at least 91%, at least 92%, at least 93%, at least 94%, at least 95%, at least 98% or at least 99% sequence identity to:
any one of SEQ ID NOs: 58, 59, 126, 127, 130, 131, 132, 134, 136, 137, 209, 210, 277, 278, 281, 282, 283, 285, 287, 288, 118, 119, 140, 141, 144, 145, 146, 148, 150, 151, 269, 270, 291, 292, 295, 296, 297, 299, 301, or 302; or
any one of SEQ ID NOs 126, 131, 134, 140, 145, 148, 277, 282, 285, 291, 296, or 299.

5. A modified Protoporphyrinogen Oxidase (PPO) enzyme or functional fragment thereof, wherein the modified Protoporphyrinogen Oxidase or functional fragment thereof comprises mutations at amino acid residues 305 and 426 of SEQ ID NO:1, or residues corresponding thereto, wherein the mutations are substitutions of 305L and 426V.

6. The modified PPO enzyme or functional fragment thereof of claim 1, wherein the modified PPO enzyme or functional fragment thereof is a PPO1 enzyme; optionally
wherein the modified PPO enzyme or functional fragment thereof, comprises an increased resistance to a compound that inhibits a PPO enzyme relative to a control PPO enzyme and/or has decreased percentage inhibition in response to a compound that inhibits a PPO enzyme relative to a control PPO enzyme; further optionally
wherein increased resistance to the compound comprises an increase of at least 2-fold, 3-fold, 4-fold, 5-fold 6-fold, 7-fold, 8-fold, 9-fold, 10-fold in resistance to the compound compared to an unmodified reference plant or part thereof, further optionally
wherein the control PPO enzyme is an unmodified PPO enzyme or a modified PPO enzyme according to SEQ ID NO: 124.

7. The modified PPO enzyme or functional fragment thereof of claim 1, wherein the modified PPO enzyme or functional fragment thereof, comprises:
(a) an amino acid sequence having at least 30%, at least 35%, at least 40%, at least 45%, at least 50%, at least 55%, at least 60%, at least 70%, at least 75%, at least 80%, at least 85%, at least 90%, at least 91%, at least 92%, at least 93%, at least 94%, at least 95%, at least 98% or at least 99% sequence identity to SEQ ID NO: 1 and at least one mutation at one or more amino acid residues selected from residues 362, 365 and/or 479 of SEQ ID NO: 1 or residues corresponding thereto: or
(b) an amino acid sequence having at least 30%, at least 35%, at least 40%, at least 45%, at least 50%, at least 55%, at least 60%, at least 70%, at least 75%, at least 80%, at least 85%, at least 90%, at least 91%, at least 92%, at least 93%, at least 94%, at least 95%, at least 98% or at least 99% sequence identity to SEQ ID NO: 2 and at least one mutation at one or more amino acid residues selected from residues 360, 363, and/or 477 of SEQ ID NO: 2 or residues corresponding thereto; or
(c) an amino acid sequence having at least 30%, at least 35%, at least 40%, at least 45%, at least 50%, at least 55%, at least 60%, at least 70%, at least 75%, at least 80%, at least 85%, at least 90%, at least 91%, at least 92%, at least 93%, at least 94%, at least 95%, at least 98% or at least 99% sequence identity to SEQ ID NO: 153 and at least one mutation at one or more amino acid residues selected from residues 328, 331 and/or 445 of SEQ ID NO: 153 or residues corresponding thereto; or
(d) an amino acid sequence having at least 30%, at least 35%, at least 40%, at least 45%, at least 50%, at least 55%, at least 60%, at least 70%, at least 75%, at least 80%, at least 85%, at least 90%, at least 91%, at least 92%, at least 93%, at least 94%, at least 95%, at least 98% or at least 99% sequence identity to SEQ ID NO: 154 and at least one mutation at one or more amino acid residues selected from residues 325, 328 and/or 442 of SEQ ID NO: 154 or residues corresponding thereto.

8. The modified PPO enzyme or functional fragment thereof of:
claim 1, wherein modified PPO enzyme or functional fragment thereof further comprises one or more further mutations at one or more amino acid residues selected from residues 305, 361, 404, 426, 431, 461, and/or 479 of SEQ ID NO: 1 or residues corresponding thereto; or
wherein:
(a) mutation at residue 305 of SEQ ID NO: 1 or residues corresponding thereto comprises a substitution to a leucine;
(b) mutation at residue 361 of SEQ ID NO: 1 or residues corresponding thereto comprises a substitution to cysteine, aspartic acid, glutamine or threonine;
(c) mutation at residue 404 of SEQ ID NO: 1 or residues corresponding thereto comprises a substitution to alanine;
(d) mutation at residue 426 of SEQ ID NO: 1 or residues corresponding thereto comprises a substitution to leucine, cysteine, methionine, threonine or valine;
(e) mutation at residue 431 of SEQ ID NO: 1 or residues corresponding thereto comprises a substitution to alanine, phenylalanine or arginine; and/or
(f) mutation at residue 461 of SEQ ID NO: 1 or residues corresponding thereto comprises a substitution to glutamine.

9. The modified PPO enzyme or functional fragment thereof:
of claim 1, wherein;
(a) the mutation at residue 362 of SEQ ID NO: 1 or residues corresponding thereto comprises a substitution to a cysteine or phenylalanine;
(b) the mutation at residue 365 of SEQ ID NO: 1 or residues corresponding thereto comprises a substitution to a methionine or a leucine; and/or
(c) the mutation at residue 479 of SEQ ID NO: 1 or residues corresponding thereto comprises a substitution to a methionine or asparagine.

10. The modified PPO enzyme or functional fragment thereof of claim 1, comprising a combination of mutations at residues:
(a) 365, 426, and 479 of SEQ ID NO:1, or residues corresponding thereto;
(b) 305, 404, 426, 431, and 479 of SEQ ID NO:1, or residues corresponding thereto;
(c) 305 and 365 of SEQ ID NO:1, or residues corresponding thereto;
(d) 305, 362 and 404 of SEQ ID NO:1, or residues corresponding thereto;
(e) 305, 361, 365, 431 and 479 of SEQ ID NO:1, or residues corresponding thereto;
(f) 305, 426 and 479 of SEQ ID NO:1, or residues corresponding thereto;
(g) 426, 461 and 479 of SEQ ID NO:1, or residues corresponding thereto;
(h) 305, 361 and 365 of SEQ ID NO:1, or residues corresponding thereto;
(i) 305, 361, 362, 426 and 479 of SEQ ID NO:1, or residues corresponding thereto;
(j) 361 and 365 of SEQ ID NO:1, or residues corresponding thereto;
(k) 365, 404, and 479 of SEQ ID NO:1, or residues corresponding thereto; or
(l) 305, 404, and 479 of SEQ ID NO:1, or residues corresponding thereto.

11. The modified PPO enzyme or functional fragment thereof of claim 6:
wherein the modified PPO enzyme or functional fragment thereof comprises or consists of a sequence according to SEQ ID NOs: 188-190, 209, 210, 248-250, 269, 270, 276-288 or 290-302.

12. The modified PPO enzyme or functional fragment thereof of claim 1, wherein the modified PPO enzyme further comprises a transit peptide; optionally
wherein the transit peptide comprises a mitochondrial transit peptide and/or a chloroplastic transit peptide; further optionally
wherein the modified PPO enzyme comprises a sequence having at least 30%, at least 35%, at least 40%, at least 45%, at least 50%, at least 55%, at least 60%, at least 70%, at least 75%, at least 80%, at least 85%, at least 90%, at least 91%, at least 92%, at least 93%, at least 94%, at least 95%, at least 98% or at least 99% sequence identity to a sequence according to any one of SEQ ID NOs: 1, 2, 4-151, or 305-320;
further optionally wherein the modified PPO enzyme comprises or consists of a sequence according to SEQ ID NOs: 37-39, 58, 59, 97-99, 118, 119, 125-137, or 139-151.

13. A nucleic acid comprising a polynucleotide encoding a modified PPO enzyme or functional fragment thereof according to claim 1; or
a recombinant vector comprising the nucleic acid.

14. A plant or part thereof comprising a modified PPO enzyme or functional fragment thereof according to claim 1,
wherein the plant or part thereof comprises an increased resistance to a compound that inhibits a PPO enzyme relative a control plant and/or has decreased percentage inhibition in response to a compound that inhibits a PPO enzyme relative to a control plant; further optionally
wherein the control plant is a wild type plant, or a plant comprising a modified PPO enzyme according to SEQ ID NO: 124.

15. A method of producing a plant or part thereof, the method comprising: modifying the plant or part thereof to comprise a PPO enzyme according to claim 1;
optionally wherein modifying the plant or part thereof comprises transforming the plant or part thereof with a nucleic acid or a recombinant vector.

16. A method of controlling undesired vegetation in the vicinity of a plant or part thereof or at a locus for growth of the plant or part thereof, the method comprising applying an effective amount of at least one PPO-inhibiting herbicide to the vicinity of the plant, the locus for growth of the plant or to the plant or part thereof, wherein said plant or part thereof comprises the plant or part thereof of claim 14, wherein the effective amount of said PPO-inhibiting herbicide controls undesired vegetation in said vicinity or at said locus.

17. Use of a compound which inhibits a PPO enzyme in combination with a plant or part thereof according to claim 14.

18. The modified PPO enzyme of claim 6, wherein the compound is a herbicide selected from the group consisting of: butafenacil, carfentrazone-ethyl, cyclopyranil, epyrifenacil (Herbicide A), flufenoximacil, flumioxazin, fomesafen, oxyfluorfen, pyraflufen-ethyl, saflufenacil (Herbicide E), sulfentrazone, tiafenacil, trifludimoxazin (Herbicide B); 3-(2-chloro-4-fluoro-5-(3-methyl-2,6-dioxo-4-trifluoromethyl-3,6-dihydropyrimidin-1(2H)-yl)phenyl)-5-methyl-4,5-dihydroisoxazole-5-carboxylic acid ethyl ester (Herbicide C); ethyl 2-[[3-[[3-chloro-5-fluoro-6-[3-methyl-2,6-dioxo-4-(trifluoromethyl)pyrimidin-1-yl]-2-pyridyl]oxy]acetate; ethyl (5S)-3-[2-chloro-5-(3,5-dimethyl-2,6-dioxo-4-thioxo-1,3,5-triazinan-1-yl)-4-fluoro-phenyl]-5-methyl-4H-isoxazole-5-carboxylate (Herbicide D); pyridin-2-ylmethyl-[(3-(2-chloro-4-fluoro-5-[3-methyl-2,6-dioxo-4-(trifluoromethyl)-3,6-dihydropyrimidin-1(2H)-yl]phenoxy)pyridine-2-yl)oxy]acetate, 2-Methoxyethyl-[(3-(2-chloro-4-fluoro-5-[3-methyl-2,6-dioxo-4-(trifluoromethyl)-3,6-dihydropyrimidin-1(2H)-yl]phenoxy)pyridine-2-yl)oxy]acetate, 2-Methoxyethyl-[(3-(2-cyan-4-fluoro-5-[3-methyl-2,6-dioxo-4-(trifluoromethyl)-3,6-dihydro-pyrimidin-1(2H)-yl]phenoxy)pyridine-2-yl)oxy]acetate and cyanomethyl-[(3-(2-bromo-4-fluoro-5-[3-methyl-2,6-dioxo-4-(trifluoromethyl)-3,6-dihydropyrimidin-1(2H)-yl]phenoxy)pyridine-2-yl)oxy]acetate; methyl 3-[2-chloro-5-[3,6-dihydro-3-methyl-2,6-dioxo-4-(trifluoromethyl)-1(2H)-pyrimidinyl]-4-fluorophenyl]-3a,4,5,6-tetrahydro-6-methyl-6aH-cyclopent[d]isoxazole-6a-carboxylate; methyl 2-[2-[2-bromo-4-fluoro-5-[3-methyl-2,6-dioxo-4-(trifluoromethyl)pyrimidin-1-yl]phenoxy]phenoxy]-2-methoxy-acetate; (2-ethoxy-2-oxo-ethyl) 1-[2-chloro-4-fluoro-5-[3-methyl-2,6-dioxo-4-(trifluoromethyl)pyrimidin-1-yl]phenoxy]cyclopropanecarboxylate, enantiomers (including enantiomer enriched mixtures) of said compounds, and/or agrochemically acceptable salts and/or esters thereof.
